(19) United States
(12) Patent Application Publication
(10) Pub. No.: US 2025/0213555 A1
(43) Pub. Date: Jul. 3, 2025

(54) SUBSTITUTED TETRAHYDROFURAN ANALOGS AS MODULATORS OF SODIUM CHANNELS

(71) Applicant: VERTEX PHARMACEUTICALS INCORPORATED, Boston, MA (US)

(72) Inventors: Elizabeth Mary BECK, Abingdon, Oxfordshire (GB); Robert PULLIN, Oxford, Oxfordshire (GB); Gorka ETXEBARRIA JARDI, Badalona (ES); Dean STAMOS, Lexington, MA (US); Yvonne SCHMIDT, San Diego, CA (US); Joseph PONTILLO, San Diego, CA (US); Stephen Andrew THOMSON, Durham, NC (US); David Matthew SHAW, Oxford, Oxfordshire (GB); Nadia M. AHMAD, Hayes, Middlesex (GB); Lidio Marx CARVALHO MEIRELES, San Marcos, CA (US); Sarah SKERRATT, Cambridge, Cambridgeshire (GB); Sara S. HADIDA RUAH, La Jolla, CA (US); Timothy Donald NEUBERT, San Diego, CA (US); Dennis James HURLEY, San Marcos, CA (US); Alexander KINTZER, Carlsbad, CA (US); Steven John DURRANT, Headington, Oxford (GB); Christopher WRAY, Berkshire (GB); Anisa Nizarali VIRANI, Thatcham, Berkshire (GB); Kiri NORTH, Oxford, Oxfordshire (GB); Reece JACQUES, Oxford, Oxfordshire (GB); Stephen Michael GEDDIS, Oxford, Oxfordshire (GB); Bhairavi GALAN, Abingdon, Oxfordshire (GB); Ronald Marcellus KNEGTEL, Abingdon, Oxfordshire (GB); Ewa Iwona CHUDYK, Wantage, Oxfordshire (GB); Joanne Louise PINDER, Didcot, Oxfordshire (GB); Bruno Artur SOUSA, Reading, Berkshire (GB); Damien FRAYSSE, Abingdon, Oxfordshire (GB); James Jun Bon MUI, Hertfordshire (GB); James Robert AUSTIN, Didcot, Oxfordshire (GB); Pierre-Henri STORCK, Abingdon, Oxfordshire (GB)

(73) Assignee: VERTEX PHARMACEUTICALS INCORPORATED, Boston, MA (US)

(21) Appl. No.: 18/566,279

(22) PCT Filed: Jun. 3, 2022
(86) PCT No.: PCT/US2022/032196

§ 371 Date: Dec. 1, 2023

Related U.S. Application Data
(60) Provisional application No. 63/197,199, filed on Jun. 4, 2021.

(51) Int. Cl. A61K 31/496; C07D 405/12; C07D 405/14; C07D 413/14; C07D 471/04; C07D 471/08; C07D 487/10; C07D 491/08; C07D 491/107; C07D 498/04; C07D 519/00

(52) U.S. Cl. CPC A61K 31/496; C07D 405/12; C07D 405/14; C07D 413/14; C07D 471/04; C07D 471/08; C07D 487/10; C07D 491/08; C07D 491/107; C07D 498/04; C07D 519/00

(57) ABSTRACT

Compounds of formula I, and pharmaceutically acceptable salts thereof, useful as inhibitors of sodium channels are provided. Also provided are pharmaceutical compositions comprising the compounds or pharmaceutically acceptable salts and methods of using the compounds, pharmaceutically acceptable salts, and pharmaceutical compositions in the treatment of various disorders, including pain.

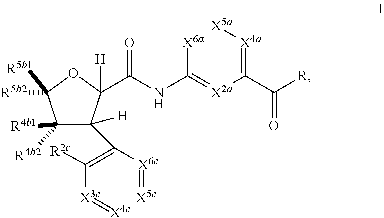

### CROSS-REFERENCE TO RELATED APPLICATIONS

This application claims the benefit of U.S. Provisional Application No. 63/197,199, filed Jun. 4, 2021, which is incorporated by reference herein in its entirety.

### BACKGROUND

Pain is a protective mechanism that allows healthy animals to avoid tissue damage and to prevent further damage to injured tissue. Nonetheless, there are many conditions where pain persists beyond its usefulness, or where patients would benefit from inhibition of pain. Neuropathic pain is a form of chronic pain caused by an injury to the sensory nerves (Dieleman, J. P., et al., Incidence rates and treatment of neuropathic pain conditions in the general population. Pain, 2008. 137 (3): p. 681-8). Neuropathic pain can be divided into two categories, pain caused by generalized metabolic damage to the nerve and pain caused by a discrete nerve injury. The metabolic neuropathies include post-herpetic neuropathy, diabetic neuropathy, and drug-induced neuropathy. Discrete nerve injury indications include post-amputation pain, post-surgical nerve injury pain, and nerve entrapment injuries like neuropathic back pain. Neuropathic pain is a major cause of disability worldwide, negatively affecting patient's sleep, mood, and functionality. Clin. Ther., 2018 40 (6): p. 828-49.

Voltage-gated sodium channels (Naᵥs) are involved in pain signaling. Naᵥs mediate the rapid upstroke of the action potential of many excitable cell types (e.g. neurons, skeletal myocytes, cardiac myocytes), and thus are involved in the initiation of signaling in those cells (Hille, Bertil, Ion Channels of Excitable Membranes, Third ed. (Sinauer Associates, Inc., Sunderland, MA, 2001)). Support for the assertion that Naᵥs play a critical and central role in pain signaling arises from (1) evaluation of the role Naᵥs plays in normal physiology, (2) pathological states arising from mutations in the Nav1.8 gene (SCN10A). (3) preclinical work in animal models, and (4) pharmacology of known Nav1.8-modulating agents. In addition, because Nav1.8 expression is restricted to peripheral neurons, particularly those that sense pain (e.g., the dorsal root ganglia), Nav1.8 inhibitors are less likely to be associated with the side effects commonly observed with other sodium channel modulators and the abuse liability associated with opioid therapies. Therefore, targeting the underlying biology of pain through selective Nav1.8 inhibition represents a novel approach to analgesic drug development that has the potential to address an urgent unmet need for safe and effective acute and chronic pain therapies. (Rush, A. M. and T. R. Cummins, Painful Research: Identification of a Small-Molecule Inhibitor that Selectively Targets Naᵥ1.8 Sodium Channels. Mol. Interv., 2007. 7 (4): p. 192-5); England, S., Voltage-gated sodium channels: the search for subtype-selective analgesics. Expert Opin. Investig. Drugs 17 (12), p. 1849-64 (2008); Krafte, D. S. and Bannon, A. W., Sodium channels and nociception: recent concepts and therapeutic opportunities. Curr. Opin. Pharmacol. 8 (1), p. 50-56 (2008)). Because of the role Naᵥs play in the initiation and propagation of neuronal signals, antagonists that reduce Naᵥ currents can prevent or reduce neural signaling and Naᵥ channels have been considered likely targets to reduce pain in conditions where hyper-excitability is observed (Chahine, M., Chatelier, A., Babich, O., and Krupp, J. J., Voltage-gated sodium channels in neurological disorders. CNS Neurol. Disord. Drug Targets 7 (2), p. 144-58 (2008)). Several clinically useful analgesics have been identified as inhibitors of Naᵥ channels. The local anesthetic drugs such as lidocaine block pain by inhibiting Naᵥ channels, and other compounds, such as carbamazepine, lamotrigine, and tricyclic antidepressants that have proven effective at reducing pain have also been suggested to act by sodium channel inhibition (Soderpalm, B., Anticonvulsants: aspects of their mechanisms of action. Eur. J. Pain 6 Suppl. A, p. 3-9 (2002); Wang, G. K., Mitchell, J., and Wang, S. Y., Block of persistent late Na⁺ currents by antidepressant sertraline and paroxetine. J. Membr. Biol. 222 (2), p. 79-90 (2008)).

The Naᵥs form a subfamily of the voltage-gated ion channel super-family and comprises 9 isoforms, designated Naᵥ1.1-Naᵥ1.9. The tissue localizations of the nine isoforms vary. Naᵥ1.4 is the primary sodium channel of skeletal muscle, and Naᵥ1.5 is the primary sodium channel of cardiac myocytes. Naᵥs 1.7, 1.8, and 1.9 are primarily localized to the peripheral nervous system, while Naᵥs 1.1, 1.2, 1.3, and 1.6 are neuronal channels found in both the central and peripheral nervous systems. The functional behaviors of the nine isoforms are similar but distinct in the specifics of their voltage-dependent and kinetic behavior (Catterall, W. A., Goldin, A. L., and Waxman, S. G., International Union of Pharmacology. XLVII. Nomenclature and structure-function relationships of voltage-gated sodium channels. Pharmacol. Rev. 57 (4), p. 397 (2005)).

Upon their discovery, Naᵥ1.8 channels were identified as likely targets for analgesia (Akopian, A. N., L. Sivilotti, and J. N. Wood, A tetrodotoxin-resistant voltage-gated sodium channel expressed by sensory neurons. Nature, 1996. 379 (6562): p. 257-62). Since then, Naᵥ1.8 has been shown to be a carrier of the sodium current that maintains action potential firing in small dorsal root ganglia (DRG) neurons (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22 (23): p. 10277-90). Naᵥ1.8 is involved in spontaneous firing in damaged neurons, like those that drive neuropathic pain (Roza, C., et al., The tetrodotoxin-resistant Na⁺ channel Naᵥ1.8 is essential for the expression of spontaneous activity in damaged sensory axons of mice. J. Physiol., 2003. 550 (Pt 3): p. 921-6; Jarvis, M. F., et al., A-803467, a potent and selective Naᵥ1.8 sodium channel blocker, attenuates neuropathic and inflammatory pain in the rat. Proc. Natl. Acad. Sci. USA, 2007. 104 (20): p. 8520-5; Joshi, S. K., et al., Involvement of the TTX-resistant sodium channel Naᵥ1.8 in inflammatory and neuropathic, but not post-operative, pain states. Pain, 2006. 123 (1-2): pp. 75-82; Lai, J., et al., Inhibition of neuropathic pain by decreased expression of the tetrodotoxin-resistant sodium channel, Naᵥ1.8i. Pain, 2002. 95 (1-2): p. 143-52; Dong, X. W., et al., Small interfering RNA-mediated selective knockdown of Naᵥ1.8 tetrodotoxin-resistant sodium channel reverses mechanical allodynia in neuropathic rats. Neuroscience, 2007. 146 (2): p. 812-21; Huang, H. L., et al., Proteomic profiling of neuromas reveals alterations in protein composition and local protein synthesis in hyper-excitable nerves. Mol. Pain, 2008. 4: p. 33; Black, J. A., et al., Multiple sodium channel isoforms and mitogen-activated protein kinases are present in painful human neuromas. Ann. Neurol., 2008. 64 (6): p. 644-53; Coward, K., et al., Immunolocalization of SNS/PN3 and NaN/SNS2 sodium channels in human pain states. Pain, 2000. 85(1-2): p. 41-50; Yiangou, Y., et al., SNS/PN3 and SNS2/NaN sodium channel-like immunoreactivity in human adult and neonate injured sensory nerves. FEBS Lett, 2000. 467(2-3): p. 249-52; Ruangsri, S., et al., Relationship of axonal voltage-gated sodium channel 1.8 (Naᵥ1.8) mRNA accumulation to sciatic nerve injury-induced painful neuropathy in rats. J. Biol. Chem. 286(46): p. 39836-47). The small DRG neurons where Naᵥ1.8 is expressed include the nociceptors involved in pain signaling. Naᵥ1.8 mediates large amplitude action potentials in small neurons of the dorsal root ganglia (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22(23): p. 10277-90). Naᵥ1.8 is necessary for rapid repetitive action potentials in nociceptors and for spontaneous activity of damaged neurons. (Choi, J. S. and S. G. Waxman, Physiological interactions between Naᵥ1.7 and Naᵥ1.8 sodium channels: a computer simulation study. J. Neurophysiol. 106(6): p. 3173-84; Renganathan, M., T. R. Cummins, and S. G. Waxman, Contribution of Na(ᵥ)1.8 sodium channels to action potential electrogenesis in DRG neurons. J. Neurophysiol., 2001. 86(2): p. 629-40; Roza, C., et al., The tetrodotoxin-resistant Na⁺ channel Naᵥ1.8 is essential for the expression of spontaneous activity in damaged sensory axons of mice. J. Physiol., 2003. 550 (Pt 3): p. 921-6). In depolarized or damaged DRG neurons, Naᵥ1.8 appears to be a driver of hyper-excitablility (Rush, A. M., et al., A single sodium channel mutation produces hyper- or hypoexcitability in different types of neurons. Proc. Natl. Acad. Sci. USA, 2006. 103(21): p. 8245-50). In some animal pain models, Naᵥ1.8 mRNA expression levels have been shown to increase in the DRG (Sun, W., et al., Reduced conduction failure of the main axon of polymodal nociceptive C-fibers contributes to painful diabetic neuropathy in rats. Brain, 135 (Pt 2): p. 359-75; Strickland, I. T., et al., Changes in the expression of Naᵥ1.7, Naᵥ1.8 and Naᵥ1.9 in a distinct population of dorsal root ganglia innervating the rat knee joint in a model of chronic inflammatory joint pain. Eur. J. Pain, 2008. 12(5): p. 564-72; Qiu, F., et al., Increased expression of tetrodotoxin-resistant sodium channels Naᵥ1.8 and Naᵥ1.9 within dorsal root ganglia in a rat model of bone cancer pain. Neurosci. Lett, 512(2): p. 61-6).

The inventors have discovered that some voltage-gated sodium channel inhibitors have limitations as therapeutic agents due to, for example, a poor therapeutic window (e.g., due to a lack of Naᵥ isoform selectivity, low potency, and/or other reasons). Accordingly, there remains a need to develop selective voltage-gated sodium channel inhibitors, such as selective Naᵥ1.8 inhibitors.

### SUMMARY

In one aspect, the invention relates to a compound described herein, or a pharmaceutically acceptable salt thereof.

In another aspect, the invention relates to a pharmaceutical composition comprising the compound, or a pharmaceutically acceptable salt thereof, and one or more pharmaceutically acceptable carriers or vehicles.

In still another aspect, the invention relates to a method of inhibiting a voltage gated sodium channel in a subject by administering the compound, pharmaceutically acceptable salt, or pharmaceutical composition to the subject.

In yet another aspect, the invention relates to a method of treating or lessening the severity in a subject of a variety of diseases, disorders, or conditions, including, but not limited to, chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, and cardiac arrhythmia, by administering the compound, pharmaceutically acceptable salt, or pharmaceutical composition to the subject.

### BRIEF DESCRIPTION OF THE DRAWINGS

FIG. 1 depicts an XRPD pattern characteristic of amorphous Compound 6.

FIG. 2 depicts an XRPD pattern characteristic of amorphous Compound 7.

FIG. 3 depicts an XRPD pattern characteristic of amorphous Compound 86.

FIG. 4 depicts an XRPD pattern characteristic of amorphous Compound 87.

FIG. 5 depicts an XRPD pattern characteristic of amorphous Compound 123.

FIG. 6 depicts an XRPD pattern characteristic of amorphous Compound 181.

FIG. 7 depicts an XRPD pattern characteristic of amorphous Compound 224.

### DETAILED DESCRIPTION

In one aspect, the invention relates to a compound of formula (I)

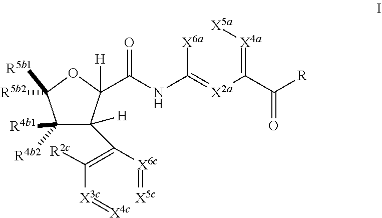

or a pharmaceutically acceptable salt thereof, wherein:
X^{2a} is N, N⁺—O, or C—R^{2a};
X^{4a} is N, N⁺—O, or C—R^{4a};
X^{5a} is N, N⁺—O, or C—R^{5a};
X^{6a} is N, N⁺—O, or C—R^{6a};
R is OR^{a} or NR^{Xa}R^{Ya};
R^{2a}, R^{4a}, R^{5a}, and R^{6a} are each independently H, halo, C₁-C₆ alkyl, C₁-C₆ haloalkyl, or —Si(C₁-C₆ alkyl)₃;
R^{a} is H or C₁-C₆ alkyl;
R^{Xa} is H or C₁-C₆ alkyl;
R^{Ya} is H, OH, C₁-C₆ alkyl, —(C₁-C₆ alkylene)-R^{Za1}, or 4-6 membered heterocyclyl optionally substituted with one or more groups independently selected from C₁-C₆ alkyl and C₁-C₆ alkoxy;
   or R^{Xa} and R^{Ya}, together with the nitrogen atom to which they are attached, form a 5-9 membered heterocyclyl, wherein said heterocyclyl is optionally substituted with one or more R^{Za2};
R^{Za1} is OH, NH₂, —NH(C₁-C₆ alkyl), —N(C₁-C₆ alkyl)₂, and 5-6 membered heterocyclyl optionally substituted with one or more groups independently selected from halo and C₁-C₆ alkyl;
each R^{Za2} is independently selected from halo, OH, C₁-C₆ alkyl, C₁-C₆ alkoxy, NH₂, —NH(C₁-C₆ alkyl), —N(C₁-C₆ alkyl)₂, and —(C₁-C₆ alkylene)-(C₁-C₆ alkoxy);
R^{4b1} and R^{4b2} are each independently H, C₁-C₆ alkyl, C₃-C₆ cycloalkyl, or C₁-C₆ haloalkyl;
R^{5b1} and R^{5b2} are each independently H, C₁-C₆ alkyl, C₃-C₆ cycloalkyl, C₁-C₆ haloalkyl, or —(C₁-C₆ alkylene)-(C₁-C₆ alkoxy);
or R^{5b1} and R^{5b2}, together with the carbon atom to which they are attached, form a 4-6 membered heterocyclyl;
X^{3c} is N or C—R^{3c};
X^{4c} is N or C—R^{4c};
X^{5c} is N or C—R^{5c};
X^{6c} is N or C—R^{6c};
R^{2c} is H, OH, halo, C₁-C₆ alkyl, C₂-C₆ alkenyl, C₁-C₆ haloalkyl, C₁-C₆ alkoxy, C₁-C₆ haloalkoxy, —(C₁-C₆ alkylene)-(C₁-C₆ alkoxy), —(C₁-C₆ alkylene)-O-(4-6 membered heterocyclyl), —O—(C₂-C₆ alkenylene)-(C₁-C₆ haloalkyl), -L¹-L²-(C₃-C₇ cycloalkyl), or —O-L³-R^{Xc}, wherein said cycloalkyl is optionally substituted with one or more groups independently selected from halo, OH, CN, C₁-C₆ alkyl, C₁-C₆ alkoxy, ═NOH, —C(O) (C₁-C₆ alkyl), and —(C₁-C₆ alkylene)-OH;
L¹ is a bond or O;
L² is a bond or C₁-C₆ alkylene;
L³ is a bond, C₁-C₆ alkylene, or C₂-C₆ alkenylene;
R^{Xc} is selected from OH, CN, C₁-C₆ alkoxy, NH₂, —NH(C₁-C₆ alkyl), —N(C₁-C₆ alkyl)₂, —NH(C₁-C₆ haloalkyl), —NH(C₁-C₆ haloalkyl)₂, —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O) (C₁-C₆ alkyl), —C(O) NH₂, —C(O) NH(C₁-C₆ alkyl), —C(O) N(C₁-C₆ alkyl)₂, —NH (4-6 membered heterocyclyl), ═NOH, ═NO(C₁-C₆ alkyl), —N═S(O)(C₁-C₆ alkyl)₂, —C(═NOH)(C₃-C₆ cycloalkyl), 4-8 membered heterocyclyl, and 5-6 membered heteroaryl, wherein said cycloalkyl is optionally substituted with one or more halo, and wherein said heterocyclyl and heteroaryl are optionally substituted with one or more groups independently selected from OH, halo,
   C₁-C₆ alkyl, C₁-C₆ alkoxy, C₁-C₆ haloalkyl, C₁-C₆ haloalkoxy, and —(C₁-C₆ alkylene)-OH;
R^{3c} is H, halo, C₁-C₆ alkyl, C₁-C₆ haloalkyl, —(C₁-C₆ alkylene)-OH, or —(C₁-C₆ alkylene)-(C₁-C₆ alkoxy);
   or wherein X^{3c} is C—R^{3c}, and R^{2c} and R^{3c}, together with the carbon atoms to which they are attached, form a ring of formula:

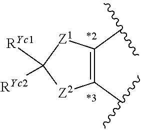

Z¹ and Z² are each, independently, O, CH₂, or CF₂;
R^{Yc1} and R^{Yc2} are each, independently, H or halo;
R^{4c} is H, halo, OH, —OBn, C₁-C₆ alkyl, C₁-C₆ alkoxy, C₁-C₆ haloalkyl, C₁-C₆ haloalkoxy, or -L¹-L²-(C₃-C₆ cycloalkyl), wherein said cycloalkyl is optionally substituted with 1-2 halo;
R^{5c} is H, halo, OH, —OBn, C₁-C₆ alkyl, C₁-C₆ haloalkyl, or -L¹-L²-(C₃-C₆ cycloalkyl), wherein said cycloalkyl is optionally substituted with 1-2 halo;
R^{6c} is H, halo, C₁-C₆ alkyl, or C₁-C₆ haloalkyl;
provided that no more than two of X^{2a}, X^{4a}, X^{5a}, and X^{6a} are N or N⁺—O;
provided that no more than one of X^{3c}, X^{4c}, X^{5c}, and X^{6c} is N; and provided that:
   R is OR^{a}; or
   R is NR^{Xa}R^{Ya}, wherein R^{Ya} is OH, —(C₁-C₆ alkylene)-R^{Za1}, or 4-6 membered heterocyclyl optionally substituted with one or more groups independently selected from C₁-C₆ alkyl and C₁-C₆ alkoxy; or
   R is NR^{Xa}R^{Ya}, wherein R^{Xa} and R^{Ya}, together with the N atom to which they are attached, form a 5-9 membered heterocyclyl, and wherein said heterocyclyl is optionally substituted with one or more R^{Za2}; or
   R^{2a}, R^{4a}, R^{5a}, or R₆ₐ is —Si(C₁-C₆ alkyl); or
   R^{5b1} or R^{5b2} is —(C₁-C₆ alkylene)-(C₁-C₆ alkoxy); or
   R^{5b1} and R^{5b2}, together with the carbon atom to which they are attached, form a 4-6 membered heterocyclyl; or
   R^{2c} is —(C₁-C₆ alkylene)-(C₁-C₆ alkoxy), —(C₁-C₆ alkylene)-O-(4-6 membered heterocyclyl), —O—(C₂-C₆ alkenylene)-(C₁-C₆ haloalkyl), or —O-L³-R^{Xc}; or
   R^{2c} is -L¹-L²-(C₃-C₇ cycloalkyl), wherein said cycloalkyl is substituted with one or more groups independently selected from OH, CN, C₁-C₆ alkyl, C₁-C₆ alkoxy, ═NOH, —C(O) (C₁-C₆ alkyl), and —(C₁-C₆ alkylene)-OH; or
   R^{3c} is —(C₁-C₆ alkylene)-OH or —(C₁-C₆ alkylene)-(C₁-C₆ alkoxy); or
   R^{2c} and R^{3c}, together with the carbon atoms to which they are attached, form a ring of formula:

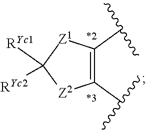

or
R^{4c} is OH, —OBn, C₁-C₆ alkoxy, C₁-C₆ haloalkoxy, or -L¹-L²-(C₃-C₆ cycloalkyl), wherein said cycloalkyl is optionally substituted with 1-2 halo; or
R^{5c} is OH, —OBn, or -L¹-L²-(C₃-C₆ cycloalkyl), wherein said cycloalkyl is optionally substituted with 1-2 halo.

In one aspect, the invention relates to a compound of formula (I′)

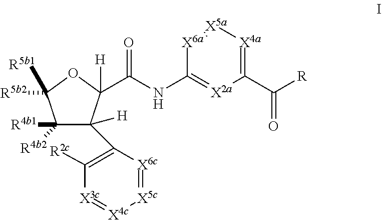

or a pharmaceutically acceptable salt thereof, wherein:
X^{2a} is N, N⁺—O, or C—R^{2a};
X^{4a} is N, N⁺—O, or C—R^{4a};
X^{5a} is N, N⁺—O, or C—R^{5a};
X^{6a} is N, N⁺—O, or C—R^{6a}; R is OR^{a} or NR^{Xa}R^{Ya};
R^{2a}, R^{4a}, R^{5a}, and R^{6a} are each independently H, halo, C₁-C₆ alkyl, C₁-C₆ haloalkyl, —Si(C₁-C₆ alkyl)₃, —Si(O—C₁-C₆ alkoxy)₃, —Si(C₁-C₆ alkyl) (O—C₁-C₆ alkoxy)₂, or —Si(C₁-C₆ alkyl)₂ (C₁-C₆ alkoxy);
R^{a} is H or C₁-C₆ alkyl;
R^{Xa} is H or C₁-C₆ alkyl;
R^{Ya} is H, OH, C₁-C₆ alkyl, —(C₁-C₆ alkylene)-R^{Za1}, or 4-6 membered heterocyclyl optionally substituted with one or more groups independently selected from C₁-C₆ alkyl and C₁-C₆ alkoxy;
   or R^{Xa} and R^{Ya}, together with the nitrogen atom to which they are attached, form a 5-9 membered heterocyclyl, wherein said heterocyclyl is optionally substituted with one or more R^{Za2};
R^{Za1} is OH, NH₂, —NH(C₁-C₆ alkyl), —N(C₁-C₆ alkyl)₂, and 5-6 membered heterocyclyl optionally substituted with one or more groups independently selected from halo and C₁-C₆ alkyl;
each R^{Za2} is independently selected from halo, OH, C₁-C₆ alkyl, C₁-C₆ alkoxy, NH₂, —NH(C₁-C₆ alkyl), —N(C₁-C₆ alkyl)₂, and —(C₁-C₆ alkylene)-(C₁-C₆ alkoxy);
R^{4b1} and R^{4b2} are each independently H, C₁-C₆ alkyl, C₃-C₆ cycloalkyl, or C₁-C₆ haloalkyl;
R^{5b1} and R^{5b2} are each independently H, C₁-C₆ alkyl, C₃-C₆ cycloalkyl, C₁-C₆ haloalkyl, or (C₁-C₆ alkylene)-(C₁-C₆ alkoxy);
   or R^{5b1} and R^{5b2}, together with the carbon atom to which they are attached, form a 4-6 membered heterocyclyl;
X^{3c} is N or C—R^{3c};
X^{4c} is N or C—R^{4c};
X^{5c} is N or C—R^{5c};
X^{6c} is N or C—R^{6c};
R^{2c} is H, OH, halo, C₁-C₆ alkyl, C₂-C₆ alkenyl, C₁-C₆ haloalkyl, C₁-C₆ alkoxy, C₁-C₆ haloalkoxy, —(C₁-C₆ alkylene)-(C₁-C₆ alkoxy), —(C₁-C₆ alkylene)-O-(4-6 membered heterocyclyl), —O—(C₂-C₆ alkenylene)-(C₁-C₆ haloalkyl), -L¹-L²-(C₃-C₇ cycloalkyl), or —O-L³-R^{Xc}, wherein said cycloalkyl is optionally substituted with one or more groups independently selected from halo, OH, CN, C₁-C₆ alkyl, C₁-C₆ alkoxy, ═NOH, —C(O) (C₁-C₆ alkyl), and —(C₁-C₆ alkylene)-OH;
L¹ is a bond or O;
L² is a bond or C₁-C₆ alkylene;
L³ is a bond, C₁-C₆ alkylene, or C₂-C₆ alkenylene;
R^{Xc} is selected from OH, CN, C₁-C₆ alkoxy, NH₂, —NH(C₁-C₆ alkyl), —N(C₁-C₆ alkyl)₂, —NH(C₁-C₆ haloalkyl), —NH(C₁-C₆ haloalkyl)₂, —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O) (C₁-C₆ alkyl), —C(O) NH₂, —C(O) NH(C₁-C₆ alkyl), —C(O) N(C₁-C₆ alkyl)₂, —NH (4-6 membered heterocyclyl), ═NOH, ═NO(C₁-C₆ alkyl), —N═S(O)(C₁-C₆ alkyl)₂, —C(═NOH)(C₃-C₆ cycloalkyl), 4-8 membered heterocyclyl, and 5-6 membered heteroaryl, wherein said cycloalkyl is optionally substituted with one or more halo, and wherein said heterocyclyl and heteroaryl are optionally substituted with one or more groups independently selected from OH, halo,
   C₁-C₆ alkyl, C₁-C₆ alkoxy, C₁-C₆ haloalkyl, C₁-C₆ haloalkoxy, and —(C₁-C₆ alkylene)-OH;
R^{3c} is H, halo, C₁-C₆ alkyl, C₁-C₆ haloalkyl, —(C₁-C₆ alkylene)-OH, or —(C₁-C₆ alkylene)-(C₁-C₆ alkoxy);
   or wherein X^{3c} is C—R^{3c}, and R^{2c} and R^{3c}, together with the carbon atoms to which they are attached, form a ring of formula:

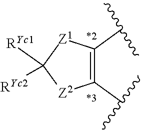

Z¹ and Z² are each, independently, O, CH₂, or CF₂;
R^{Yc1} and R^{Yc2} are each, independently, H or halo;
R^{4c} is H, halo, OH, —OBn, C₁-C₆ alkyl, C₁-C₆ alkoxy, C₁-C₆ haloalkyl, C₁-C₆ haloalkoxy, or -L¹-L²-(C₃-C₆ cycloalkyl), wherein said cycloalkyl is optionally substituted with 1-2 halo;
R^{5c} is H, halo, OH, —OBn, C₁-C₆ alkyl, C₁-C₆ haloalkyl, or -L¹-L²-(C₃-C₆ cycloalkyl), wherein said cycloalkyl is optionally substituted with 1-2 halo;
R^{6c} is H, halo, C₁-C₆ alkyl, or C₁-C₆ haloalkyl;
provided that no more than two of X^{2a}, X^{4a}, X^{5a}, and X^{6a} are N or N⁺—O;
provided that no more than one of X^{3c}, X^{4c}, X^{5c}, and X^{6c} is N; and
provided that:
   R is OR^{a}; or
   R is NR^{Xa}R^{Ya}, wherein R^{Ya} is OH, —(C₁-C₆ alkylene)-R^{Za1}, or 4-6 membered heterocyclyl optionally substituted with one or more groups independently selected from C₁-C₆ alkyl and C₁-C₆ alkoxy; or
   R is NR^{Xa}R^{Ya}, wherein R^{Xa} and R^{Ya}, together with the N atom to which they are attached, form a 5-9 membered heterocyclyl, and wherein said heterocyclyl is optionally substituted with one or more R^{Za2}; or
   R^{2a}, R^{4a}, R^{5a}, or R^{6a} is —Si(C₁-C₆ alkyl); or
   R^{5b1} or R^{5b2} is —(C₁-C₆ alkylene)-(C₁-C₆ alkoxy); or
   R^{5b1} and R^{5b2}, together with the carbon atom to which they are attached, form a 4-6 membered heterocyclyl; or
   R^{2c} is —(C₁-C₆ alkylene)-(C₁-C₆ alkoxy), —(C₁-C₆ alkylene)-O-(4-6 membered heterocyclyl), —O—(C₂-C₆ alkMenylene)-(C₁-C₆ haloalkyl), or —O-L³-R^{Xc}; or
   R^{2c} is -L¹-L²-(C₃-C₇ cycloalkyl), wherein said cycloalkyl is substituted with one or more groups independently selected from OH, CN, C₁-C₆ alkyl, C₁-C₆ alkoxy, ═NOH, —C(O) (C₁-C₆ alkyl), and —(C₁-C₆ alkylene)-OH; or
   R^{3c} is —(C₁-C₆ alkylene)-OH or —(C₁-C₆ alkylene)-(C₁-C₆ alkoxy); or
   R^{2c} and R^{3c}, together with the carbon atoms to which they are attached, form a ring of formula:

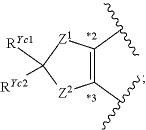

or
R^{4c} is OH, —OBn, C₁-C₆ alkoxy, C₁-C₆ haloalkoxy, or -L¹-L²-(C₃-C₆ cycloalkyl), wherein said cycloalkyl is optionally substituted with 1-2 halo; or
R^{5c} is OH, —OBn, or -L¹-L²-(C₃-C₆ cycloalkyl), wherein said cycloalkyl is optionally substituted with 1-2 halo.

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito: 1999, and “March's Advanced Organic Chemistry,” 5th Ed., Ed.: Smith, M. B. and March, J., John Wiley & Sons, New York: 2001, the entire contents of which are hereby incorporated by reference.

As used herein, the term “compounds of the invention” refers to the compounds of formula (I), and all of the embodiments thereof (e.g., formulas (I-A), (I-B), (I-C) etc.), as described herein, and to the compounds identified in Table A.

As described herein, the compounds of the invention comprise multiple variable groups (e.g., R, X^{2a}, R^{5b1}, etc.). As one of ordinary skill in the art will recognize, combinations of groups envisioned by this invention are those combinations that result in the formation of stable or chemically feasible compounds. The term “stable,” in this context, refers to compounds that are not substantially altered when subjected to conditions to allow for their production, detection, and optionally their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The chemical structures depicted herein are intended to be understood as they would be understood by one of ordinary skill in the art. For example, with respect to formulas (I), (I-A), (I-B), and (I-C), X^{5a} and X^{6a} are connected by a double bond, and X^{4c} and X^{5c} are connected by a single bond, even though the bonds between these groups may be obscured by the atom labels in the chemical structures. Moreover, a substituent depicted as “CF₃” or “F₃C” in a chemical structure refers to a trifluoromethyl substituent, regardless of which depiction appears in the chemical structure.

As used herein, the term “halo” means F, Cl, Br or I.

As used herein, the term “alkyl” refers to a straight or branched hydrocarbon chain radical group consisting solely of carbon and hydrogen atoms, containing no unsaturation, and having the specified number of carbon atoms, which is attached to the rest of the molecule by a single bond. For example, a “C₁-C₆ alkyl” group is an alkyl group having between one and six carbon atoms.

As used herein, the term “alkenyl” refers to a straight or branched hydrocarbon chain radical group consisting solely of carbon and hydrogen atoms, containing one or more carbon-carbon double bonds, and having the specified number of carbon atoms, which is attached to the rest of the molecule by a single bond. For example, a “C₂-C₆ alkenyl” group is an alkenyl group having between two and six carbon atoms.

As used herein, the term “cycloalkyl” refers to a stable, non-aromatic, mono- or bicyclic (fused, bridged, or spiro) saturated hydrocarbon radical consisting solely of carbon and hydrogen atoms, having the specified number of carbon ring atoms, and which is attached to the rest of the molecule by a single bond. For example, a “C₃-C₈ cycloalkyl” group is a cycloalkyl group having between three and eight carbon atoms.

As used herein, the term “alkoxy” refers to a radical of the formula —ORₐ where Rₐ is an alkyl group having the specified number of carbon atoms. For example, a “C₁-C₆ alkoxy” group is a radical of the formula —ORₐ where Rₐ is an alkyl group having the between one and six carbon atoms.

As used herein, the term “haloalkyl” refers to an alkyl group having the specified number of carbon atoms, wherein one or more of the hydrogen atoms of the alkyl group are replaced by halo groups. For example, a “C₁-C₆ haloalkyl” group is an alkyl group having between one and six carbon atoms, wherein one or more of the hydrogen atoms of the alkyl group are replaced by halo groups.

As used herein, the term “haloalkoxy” refers to an alkoxy group having the specified number of carbon atoms, wherein one or more of the hydrogen atoms of the of the alkyl group are replaced by halo groups.

As used herein, the term “alkylene” refers to a divalent, straight or branched hydrocarbon chain radical group consisting solely of carbon and hydrogen atoms, containing no unsaturation, and having the specified number of carbon atoms, which is attached to the rest of the molecule by two single bonds. For example, a “C₁-C₆ alkylene” group is an alkylene group having between one and six carbon atoms.

As used herein, the term “alkenylene” refers to a divalent, straight or branched hydrocarbon chain radical group consisting solely of carbon and hydrogen atoms, containing one or more carbon-carbon double bonds, and having the specified number of carbon atoms, which is attached to the rest of the molecule by two single bonds. For example, a “C₂-C₆ alkenylene” is an alkenylene group having between one and six carbon atoms.

As used herein, the term “heterocycle,” “heterocyclyl,” “heterocycloaliphatic,” “heterocycloalkyl,” or “heterocyclic” refers to non-aromatic, monocyclic, bicyclic, or tricyclic ring systems in which one or more ring atoms in one or more ring members is an independently selected heteroatom. Heterocyclic rings can be saturated, or can contain one or more unsaturated bonds. In some embodiments, the “heterocycle,” “heterocyclyl,” “heterocycloaliphatic,” “heterocycloalkyl,” or “heterocyclic” group has the indicated number of ring members, in which one or more ring members is a heteroatom independently selected from oxygen, sulfur, nitrogen, or phosphorus, and each ring in the ring system contains 3 to 7 ring members. For example, a 6-membered heterocyclyl includes a total of 6 ring members, at least one of which is a heteroatom selected from N, S, O, and P.

As used herein, the term “heteroaryl” refers to monocyclic, bicyclic, and tricyclic ring systems having the indicated number of ring members, wherein at least one ring in the system is aromatic, at least one ring in the system contains one or more heteroatoms selected from nitrogen, sulfur, oxygen, and phosphorous, and wherein each ring in the system contains 3 to 7 ring members. For example, a 6-membered heteroaryl includes a total of 6 ring members, at least one of which is a heteroatom selected from N, S, O, and P. The term “heteroaryl” may be used interchangeably with the term “heteroaryl ring” or the term “heteroaromatic”.

As used herein, labels such as “*2” and “*3”, such as those shown in the following structure, designate the atoms to which the corresponding R groups (in this case, the R^{2c} and R^{3c} groups, respectively) are attached.

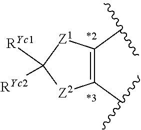

Unless otherwise specified, the compounds of the invention, whether identified by chemical name or chemical structure, include all stereoisomers (e.g., enantiomers and diastereomers), double bond isomers (e.g., (Z) and (E)), conformational isomers, and tautomers of the compounds identified by the chemical names and chemical structures provided herein. In addition, single stereoisomers, double bond isomers, conformational isomers, and tautomers as well as mixtures of stereoisomers, double bond isomers, conformational isomers, and tautomers are within the scope of the invention.

As used herein, in any chemical structure or formula, a non-bold, straight bond attached to a stereocenter of a compound, such as in

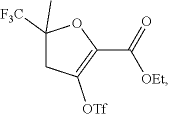 [SMILES: CCOC(=O)C1=C(OS(=O)(=O)C(F)(F)F)CC(C)(C(F)(F)F)O1]

denotes that the configuration of the stereocenter is unspecified. The compound may have any configuration, or a mixture of configurations, at the stereocenter.

As used herein, in any chemical structure or formula, a non-bold, wavy bond (e.g. “ [symbol] ”) attached to a double bond within a compound, such as in

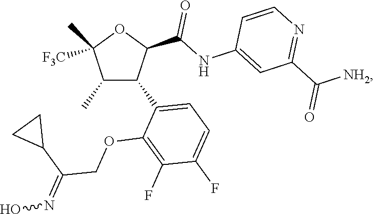 [SMILES: C[C@H]1[C@@H](c2ccc(F)c(F)c2OCC(=NO)C2CC2)[C@H](C(=O)Nc2ccnc(C(N)=O)c2)O[C@@]1(C)C(F)(F)F]

denotes that the compound was isolated as a mixture of geometric isomers (i.e., a mixture of compounds having (E) and (Z) stereochemistry around the double bond). Compounds isolated as a mixture of geometric isomers do not specify the stereochemical configuration of the double bond the recited chemical names. Other stereocenters of known configuration within the compound may bear the appropriate stereochemical designation within the recited chemical name. By way of example, the above-referenced compound is recited as 4-((2R,3S,4S,5R)-3-(2-(2-cyclopropyl-2-(hydroxyimino)ethoxy)-3,4-difluorophenyl)-4,5-dimethyl-5-(trifluoromethyl)tetrahydrofuran-2-carboxamido) picolinamide within Example 21.

As used herein, in any chemical structure or formula, a bold or hashed straight bond attached to a stereocenter of a compound, such as in

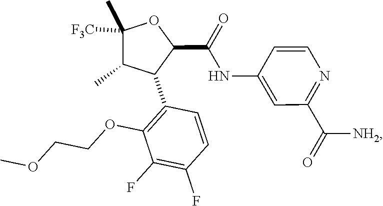

denotes the relative stereochemistry of the stereocenter, relative to other stereocenter(s) to which bold or hashed straight bonds are attached.

As used herein, in any chemical structure or formula, a bold or hashed wedge bond attached to a stereocenter of a compound, such as in

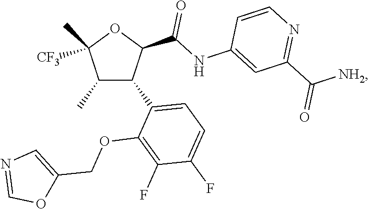 [SMILES: C[C@H]1[C@@H](c2ccc(F)c(F)c2OCc2cnco2)[C@H](C(=O)Nc2ccnc(C(N)=O)c2)O[C@@]1(C)C(F)(F)F]

denotes the absolute stereochemistry of the stereocenter, as well as the relative stereochemistry of the stereocenter, relative to other stereocenter(s) to which bold or hashed wedge bonds are attached.

As used herein, the prefix “rac-,” when used in connection with a chiral compound, refers to a racemic mixture of the compound. In a compound bearing the “rac-” prefix, the (R)- and (S)-designators in the chemical name reflect the relative stereochemistry of the compound.

As used herein, the prefix “rel-,” when used in connection with a chiral compound, refers to a single enantiomer of unknown absolute configuration. In a compound bearing the “rel-” prefix, the (R)- and (S)-designators in the chemical name reflect the relative stereochemistry of the compound, but do not necessarily reflect the absolute stereochemistry of the compound. In some instances, the prefix “rel-” is used with compounds having geometric isomerism around a double bond (e.g. —C═C—, —C═N—, etc.) to indicate the relative stereochemical configuration of the geometric isomer (i.e. (E) or (Z) stereochemistry). Where the relative stereochemistry of a given stereocenter is unknown, no stereochemical designator is provided. In some instances, the absolute configuration of some stereocenters is known, while only the relative configuration of the other stereocenters is known. In these instances, the stereochemical designators associated with the stereocenters of known absolute configuration are marked with an asterisk (*), e.g., (R*)— and (S*)—, while the stereochemical designators associated with stereocenters of unknown absolute configuration are not so marked. The unmarked stereochemical designators associated with the stereocenters of unknown absolute configuration reflect the relative stereochemistry of those stereocenters with respect to other stereocenters of unknown absolute configuration, but do not necessarily reflect the relative stereochemistry with respect to the stereocenters of known absolute configuration.

As used herein, the term “compound,” when referring to the compounds of the invention, refers to a collection of molecules having identical chemical structures, except that there may be isotopic variation among the constituent atoms of the molecules. The term “compound” includes such a collection of molecules without regard to the purity of a given sample containing the collection of molecules. Thus, the term “compound” includes such a collection of molecules in pure form, in a mixture (e.g., solution, suspension, colloid, or pharmaceutical composition, or dosage form) with one or more other substances, or in the form of a hydrate, solvate, or co-crystal.

As used herein, the term “amorphous” refers to a solid material having no long-range order in the position of its molecules. Amorphous solids are generally glasses or supercooled liquids in which the molecules are arranged in a random manner so that there is no well-defined arrangement, e.g., molecular packing, and no long-range order. Amorphous solids are generally rather isotropic, i.e., exhibit similar properties in all directions and do not have definite melting points. Instead, they typically exhibit a glass transition temperature which marks a transition from glassy amorphous state to supercooled liquid amorphous state upon heating. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See US 2004/0006237 for a comparison of XRPDs of an amorphous material and crystalline material. In some embodiments, a solid material may comprise an amorphous compound, and the material may, for example, be characterized by a lack of sharp characteristic crystalline peak(s) in its XRPD spectrum (i.e., the material is not crystalline, but is amorphous, as determined by XRPD). Instead, one or several broad peaks (e.g., halos) may appear in the XRPD pattern of the material. See US 2004/0006237 for a representative comparison of XRPDs of an amorphous material and crystalline material. A solid material, comprising an amorphous compound, may be characterized by, for example, a wider temperature range for the melting of the solid material, as compared to the range for the melting of a pure crystalline solid. Other techniques, such as, for example, solid state NMR may also be used to characterize crystalline or amorphous forms.

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

As used herein in the specification and claims, “H” refers to hydrogen and includes any stable isotope of hydrogen, namely 1H and D. In the Examples, where an atom is designated as “H,” no effort was made to enrich that atom in a particular isotope of hydrogen, and therefore a person of ordinary skill in the art would understand that such hydrogen atom likely was present at approximately the natural abundance isotopic composition of hydrogen.

As used herein, “1H” refers to protium. Where an atom in a compound of the invention, or a pharmaceutically acceptable salt thereof, is designated as protium, protium is present at the specified position with at least the natural abundance concentration of protium.

As used herein, “D,” “d,” and “2H” refer to deuterium.

In some embodiments, the compounds of the invention, and pharmaceutically acceptable salts thereof, include each constituent atom at approximately the natural abundance isotopic composition of the specified element.

In some embodiments, the compounds of the invention, and pharmaceutically acceptable salts thereof, include one or more atoms having an atomic mass or mass number which differs from the atomic mass or mass number of the most abundant isotope of the specified element (“isotope-labeled” compounds and salts). Examples of stable isotopes which are commercially available and suitable for the invention include without limitation isotopes of hydrogen, carbon, nitrogen, oxygen, and phosphorus, for example ²H, ¹³C, ¹⁵N, ¹⁸O, ¹⁷O, and ³¹P, respectively.

The isotope-labeled compounds and salts can be used in a number of beneficial ways, including as medicaments. In some embodiments, the isotope-labeled compounds and salts are deuterium (²H)-labeled. Deuterium (²H)-labeled compounds and salts are therapeutically useful with potential therapeutic advantages over the non-²H-labeled compounds. In general, deuterium (²H)-labeled compounds and salts can have higher metabolic stability as compared to those that are not isotope-labeled owing to the kinetic isotope effect described below. Higher metabolic stability translates directly into an increased in vivo half-life or lower dosages, which under most circumstances would represent a preferred embodiment of the present invention. The isotope-labeled compounds and salts can usually be prepared by carrying out the procedures disclosed in the synthesis schemes, the Examples and the related description, replacing a non-isotope-labeled reactant by a readily available isotope-labeled reactant.

The deuterium (²H)-labeled compounds and salts can manipulate the rate of oxidative metabolism of the compound by way of the primary kinetic isotope effect. The primary kinetic isotope effect is a change of the rate for a chemical reaction that results from exchange of isotopic nuclei, which in turn is caused by the change in ground state energies of the covalent bonds involved in the reaction. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For example, if deuterium is bonded to a carbon atom at a non-exchangeable position, rate differences of k_{H}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of an isotope (e.g., deuterium) incorporated at a given position of an isotope-labeled compound of the invention, or a pharmaceutically acceptable salt thereof, may be defined by the isotopic enrichment factor. The term “isotopic enrichment factor,” as used herein, means the ratio between the abundance of an isotope at a given position in an isotope-labeled compound (or salt) and the natural abundance of the isotope.

Where an atom in a compound of the invention, or a pharmaceutically acceptable salt thereof, is designated as deuterium, such compound (or salt) has an isotopic enrichment factor for such atom of at least 3000 (˜45% deuterium incorporation). In some embodiments, the isotopic enrichment factor is at least 3500 (˜52.5% deuterium incorporation), at least 4000 (˜60% deuterium incorporation), at least 4500 (˜67.5% deuterium incorporation), at least 5000 (˜75% deuterium incorporation), at least 5500 (˜82.5% deuterium incorporation), at least 6000 (˜90% deuterium incorporation), at least 6333.3 (˜95% deuterium incorporation), at least 6466.7 (˜97% deuterium incorporation), at least 6600 (˜99% deuterium incorporation), or at least 6633.3 (˜99.5% deuterium incorporation).

In some embodiments, the invention relates to a compound of formula (I-A)

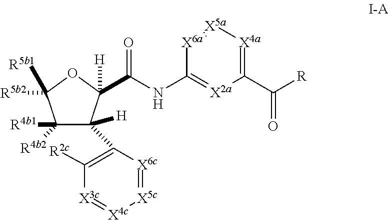

or a pharmaceutically acceptable salt thereof, wherein R, X^{2a}, X^{5a}, X^{6a}, R^{4b1}, R^{4b2}, R^{5b1}, R^{5b2}, X^{3c}, X^{4c}, X^{5c}, X^{6c}, and R^{2c} are defined as set forth above in connection with formula (I).

In some embodiments, the invention relates to a compound of formula (I-A-1)

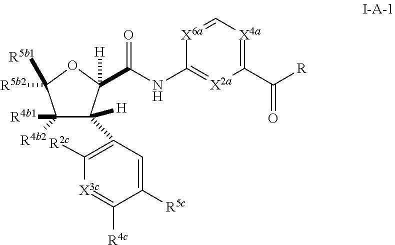

or a pharmaceutically acceptable salt thereof, wherein R, X^{2a}, X^{4a}, X^{6a}, R^{4b1}, R^{4b2}, R^{5b1}, R^{5b2}, X^{3c}, R^{2c}, R^{4c}, and R^{5c} are defined as set forth above in connection with formula (I).

In some embodiments, the invention relates to a compound of formula (I-A-2)

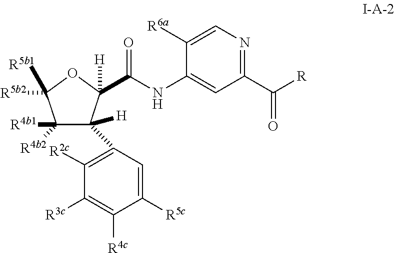

or a pharmaceutically acceptable salt thereof, wherein R, R^{6a}, R^{4b1}, R^{4b2}, R^{5b1}, R^{5b2}, R^{2c}, R^{3c}, R^{4c}, and R^{5c} are defined as set forth above in connection with formula (I).

In some embodiments, the invention relates to a compound of formula (I-A-3)

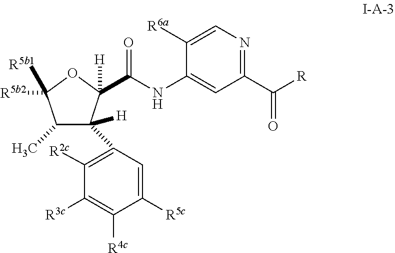

or a pharmaceutically acceptable salt thereof, wherein R, R^{6a}, R^{5b1}, R^{5b2}, R^{2c}, R^{3c}, R^{4c}, and R^{5c} are defined as set forth above in connection with formula (I).

In some embodiments, the invention relates to a compound of formula (I-A-4)

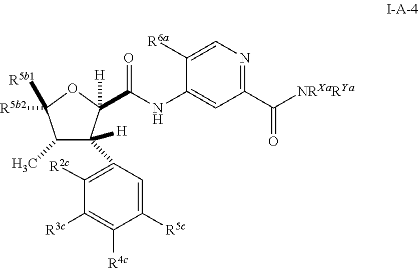

or a pharmaceutically acceptable salt thereof, wherein R^{Xa}, R^{Ya}, R^{6a}, R^{5b1}, R^{5b2}, R^{2c}, R^{3c}, and R^{4c} are defined as set forth above in connection with formula (I).

In some embodiments, the invention relates to a compound of formula (I-B)

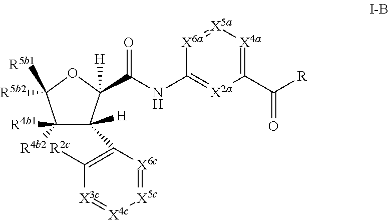

or a pharmaceutically acceptable salt thereof, wherein R, X^{2a}, X^{4a}, X^{5a}, X^{6a}, R^{4b1}, R^{4b2}, R^{5b1}, R^{5b2}, X^{3c}, X^{4c}, X^{5c}, X^{6c}, and R^{2c} are defined as set forth above in connection with formula (I).

In some embodiments, the invention relates to a compound of formula (I-B-1)

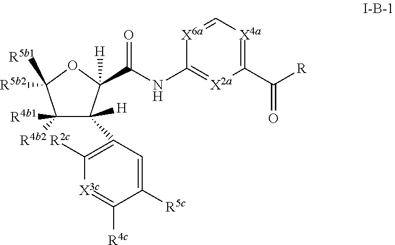

or a pharmaceutically acceptable salt thereof, wherein R, X^{2a}, X^{4a}, X^{6a}, R^{4b1}, R^{4b2}, R^{5b1}, R^{5b2}, X^{3c}, R^{2c}, R^{4c} and R^{5c} are defined as set forth above in connection with formula (I).

In some embodiments, the invention relates to a compound of formula (I-B-2)

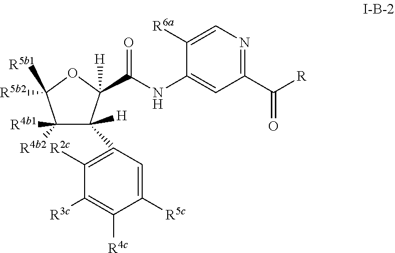

or a pharmaceutically acceptable salt thereof, wherein R, R^{6a}, R^{4b1}, R^{4b2}, R^{5b1}, R^{5b2}, R^{2c}, R^{3c}, R^{4c}, and R^{5c} are defined as set forth above in connection with formula (I).

In some embodiments, the invention relates to a compound of formula (I-B-3)

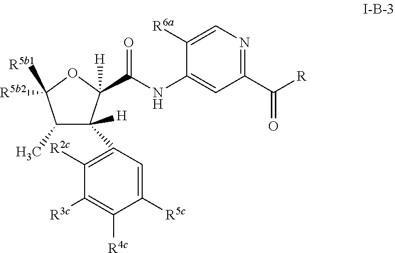

or a pharmaceutically acceptable salt thereof, wherein R, R^{6a}, R^{5b1}, R^{5b2}, R^{2c}, R^{3c}, R^{4c}, and R^{5c} are defined as set forth above in connection with formula (I).

In some embodiments, the invention relates to a compound of formula (I-B-4)

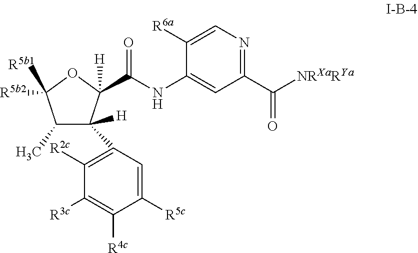

or a pharmaceutically acceptable salt thereof, wherein R^{Xa}, R^{Ya}, R^{6a}, R^{5b1}, R^{5b2}, R^{2c}, R^{3c}, and R⁴ are defined as set forth above in connection with formula (I).

In some embodiments, the invention relates to a compound of formula (I-C)

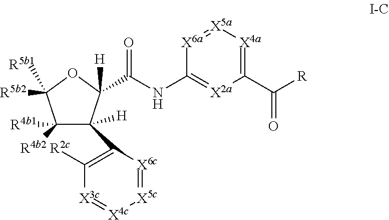

or a pharmaceutically acceptable salt thereof, wherein R, X^{2a}, X^{4a}, X^{5a}, X^{6a}, R^{4b1}, R^{4b2}, R^{5b1}, R⁵⁶², X^{3c}, X^{4c} X^{5c}, X^{6c}, and R^{2c} are defined as set forth above in connection with formula (I).

In some embodiments, the invention relates to a compound of formula (I-C-1)

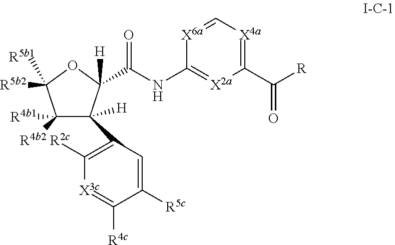

or a pharmaceutically acceptable salt thereof, wherein R, X^{2a}, X^{4a}, X^{6a}, R^{4b1}, R^{4b2}, R^{5b1}, R^{5b2}, X^{3c}, R^{2c}, R^{4c}, and R^{5c} are defined as set forth above in connection with formula (I).

In some embodiments, the invention relates to a compound of formula (I-C-2)

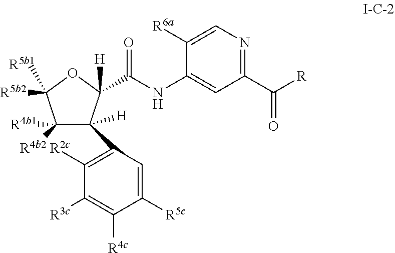

or a pharmaceutically acceptable salt thereof, wherein R, R^{6a}, R^{4b1}, R^{4b2}, R^{5b1}, R^{5b2}, R^{2c}, R^{3c}, R^{4c}, and R^{5c} are defined as set forth above in connection with formula (I).

In some embodiments, the invention relates to a compound of formula (I-C-3)

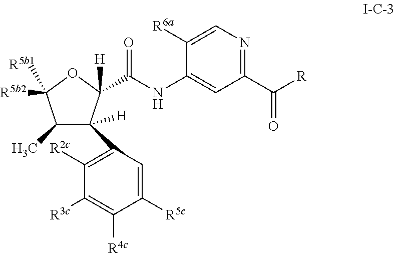

or a pharmaceutically acceptable salt thereof, wherein R, R^{6a}, R^{5b1}, R^{5b2}, R^{2c}, R^{3c}, R^{4c}, and R^{5c} are defined as set forth above in connection with formula (I).

In some embodiments, the invention relates to a compound of formula (I-C-4)

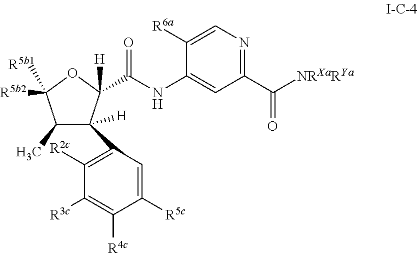

or a pharmaceutically acceptable salt thereof, wherein R^{Xa}, R^{Ya}, R^{6a}, R^{5b1}, R^{5b2}, R^{2c}, R^{3c}, and R^{4c} are defined as set forth above in connection with formula (I).

In some embodiments, the invention relates to a compound of any one of formulas (I), (I-A), (I-A-1), (I-B), (I-B-1), (I-C), and (I-C-1) or a pharmaceutically acceptable salt thereof, wherein X^{2a} is N or C—R^{2a}. In other embodiments, X^{2a} is N. In other embodiments, X^{2a} is C—R^{2a}. In other embodiments, X^{2a} is C—R^{2a}, and R^{2a} is H.

In some embodiments, the invention relates to a compound of any one of formulas (I), (I-A), (I-A-1), (I-B), (I-B-1), (I-C), and (I-C-1), or a pharmaceutically acceptable salt thereof, wherein X^{4a} is N, N⁺—O⁻, or C—R^{4a}. In other embodiments, X^{4a} is N, N⁺—O, or C—R^{4a}; and R^{4a} is H or halo. In other embodiments, X^{4a} is N. In other embodiments, X^{4a} is N⁺—O. In other embodiments, X^{4a} is C—R^{4a}. In other embodiments, X^{4a} is C—R^{4a}, and R^{4a} is H or halo. In other embodiments, X^{4a} is C—R^{4a}, and R^{4a} is H or F. In other embodiments, X^{4a} is C—R^{4a}, and R^{4a} is H. In other embodiments, X^{4a} is C—R^{4a}, and R^{4a} is F.

In some embodiments, the invention relates to a compound of any one of formulas (I), (I-A), (I-B), and (I-C), or a pharmaceutically acceptable salt thereof, wherein X^{5a} is C—R^{5a}. In other embodiments, X^{5a} is C—R^{5a}, and R^{5a} is H.

In some embodiments, the invention relates to a compound of any one of formulas (I), (I-A), (I-A-1), (I-B), (I-B-1), (I-C), and (I-C-1), or a pharmaceutically acceptable salt thereof, wherein X^{6a} is N or C—R^{6a}. In other embodiments, X^{6a} is N or C—R^{6a}, and R^{6a} is H, halo, C₁-C₆ alkyl, or —Si(C₁-C₆ alkyl)₃. In other embodiments, X^{6a} is N. In other embodiments, X^{6a} is C—R^{6a}. In other embodiments, X^{6a} is C—R^{6a}, and R^{6a} is H, halo, C₁-C₆ alkyl, or —Si(C₁-C₆ alkyl)₃. In other embodiments, X^{6a} is C—R^{6a}, and R^{6a} is H. In other embodiments, X^{6a} is C—R^{6a}, and R^{6a} is halo. In other embodiments, X^{6a} is C—R^{6a}, and R^{6a} is C₁-C₆ alkyl. In other embodiments, X^{6a} is C—R^{6a}, and R^{6a} is —Si(C₁-C₆ alkyl)₃. In other embodiments, X^{6a} is C—R^{6a}, and R^{6a} is H, F, CH₃, or —Si(CH₃)₃. In other embodiments, X^{6a} is C—R^{6a}, and R^{6a} is F. In other embodiments, X^{6a} is C—R^{6a}, and R^{6a} is CH₃. In other embodiments, X^{6a} is C—R^{6a}, and R^{6a} is —Si(CH₃)₃.

In some embodiments, the invention relates to a compound of any one of formulas (I-A-2), (I-A-3), (I-A-4), (I-B-2), (I-B-3), (I-B-4), (I-C-2), (I-C-3), and (I-C-4), or a pharmaceutically acceptable salt thereof, wherein R^{6a} is H, halo, C₁-C₆ alkyl, or —Si(C₁-C₆ alkyl)₃. In other embodiments, R^{6a} is H. In other embodiments, R^{6a} is halo. In other embodiments, R^{6a} is C₁-C₆ alkyl. In other embodiments, R^{6a} is —Si(C₁-C₆ alkyl)₃. In other embodiments, R^{6a} is H, F, CH₃, or —Si(CH₃)₃. In other embodiments, R^{6a} is F. In other embodiments, R^{6a} is CH₃. In other embodiments, R^{6a} is —Si(CH₃)₃.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), and (I-C-3), or a pharmaceutically acceptable salt thereof, wherein R is OR^{a} or NR^{Xa}R^{Ya}, R^{a} is H or C₁-C₆ alkyl, R^{Xa} is H or C₁-C₆ alkyl, R^{Ya} is H, OH, C₁-C₆ alkyl, —(C₁-C₆ alkylene)-R^{Za1}, or 4-6 membered heterocyclyl optionally substituted with one or more groups independently selected from C₁-C₆ alkyl and C₁-C₆ alkoxy, and R^{Za1} is OH, —NH(C₁-C₆ alkyl), —N(C₁-C₆ alkyl)₂, and 5-6 membered heterocyclyl optionally substituted with one or more groups independently selected from halo and C₁-C₆ alkyl. In other embodiments, R is OR^{a}. In other embodiments, R is OR^{a}, and R^{a} is H. In other embodiments, R is NR^{Xa}R^{Ya}. In other embodiments, R is NR^{Xa}R^{Ya}, and R^{Xa} is H or CH₃, R^{Ya} is H, OH, CH₃, —(C₁-C₂ alkylene)-R^{Za1}, or 4-6 membered heterocyclyl optionally substituted with one or more groups independently selected from CH₃, —OCH₃, and —OCH₂CH₃, and R^{Za1} is OH, —NH(CH₃), —N(CH₃)₂, and 5-6 membered heterocyclyl optionally substituted with one or more groups independently selected from F and CH₃. In other embodiments, R^{Xa} is H. In other embodiments, R^{Xa} is CH₃. In other embodiments, R^{Ya} is H. In other embodiments, R^{Ya} is OH. In other embodiments, R^{Ya} is CH₃. In other embodiments, R^{Ya} is —(C₁-C₂ alkylene)-R^{Za1}, and R^{Za1} is OH, —NH(CH₃), —N(CH₃)₂, and 5-6 membered heterocyclyl optionally substituted with one or more groups independently selected from F and CH₃. In other embodiments, R^{Ya} is —(C₁-C₂ alkylene)-R^{Za1}, and R^{Za1} is OH. In other embodiments, R^{Ya} is —(C₁-C₂ alkylene)-R^{Za1}, and R^{Za1} is-NH(CH₃). In other embodiments, R^{Ya} is —(C₁-C₂ alkylene)-R^{Za1}, and R^{Za1} is —N(CH₃)₂. In other embodiments, R^{Ya} is —(C₁-C₂ alkylene)-R^{Za1}, and R^{Za1} is 5-6 membered heterocyclyl optionally substituted with one or more groups independently selected from F and CH₃. In other embodiments, R^{Ya} is —(C₁-C₂ alkylene)-R^{Za1}, and R^{Za1} is an unsubstituted 5-6 membered heterocyclyl. In other embodiments, R^{Ya} is —(C₁-C₂ alkylene)-R^{Za1}, and R^{Za1} is 5-6 membered heterocyclyl substituted with one or more groups independently selected from F and CH₃. In other embodiments, R^{Ya} is —(C₁-C₂ alkylene)-R^{Za1}, and R^{Za1} is 5-6 membered heterocyclyl substituted with one CH₃. In other embodiments, R^{Ya} is —(C₁-C₂ alkylene)-R^{Za1}, and R^{Za1} is 5-6 membered heterocyclyl substituted with two F. In other embodiments, R^{Ya} is —(C₁-C₂ alkylene)-R^{Za1}, and R^{Za1} is 5-6 membered heterocyclyl substituted with one CH₃ and two F. In other embodiments, R^{Ya} is 4-6 membered heterocyclyl optionally substituted with one or more groups independently selected from CH₃, —OCH₃, and —OCH₂CH₃. In other embodiments, R^{Ya} is 4-6 membered heterocyclyl optionally substituted with one CH₃. In other embodiments, R^{Ya} is 4-6 membered heterocyclyl optionally substituted with one-OCH₃. In other embodiments, R^{Ya} is 4-6 membered heterocyclyl optionally substituted with one CH₃ and one —OCH₃. In other embodiments, R^{Ya} is 4-6 membered heterocyclyl optionally substituted with one CH₃ and one-OCH₂CH₃.

In some embodiments, the invention relates to a compound of any one of formulas (I-A-4), (I-B-4), and (I-C-4), or a pharmaceutically acceptable salt thereof, wherein R^{Xa} is H or CH₃, R^{Ya} is H, OH, CH₃, —(C₁-C₂ alkylene)-R^{Za1}, or 4-6 membered heterocyclyl optionally substituted with one or more groups independently selected from CH₃, —OCH₃, and —OCH₂CH₃, and R^{Za1} is OH, —NH(CH₃), —N(CH₃)₂, and 5-6 membered heterocyclyl optionally substituted with one or more groups independently selected from F and CH₃. In other embodiments, R^{Xa} is H. In other embodiments, R^{Xa} is CH₃. In other embodiments, R^{Ya} is H. In other embodiments, R^{Ya} is OH. In other embodiments, R^{Ya} is CH₃. In other embodiments, R^{Ya} is —(C₁-C₂ alkylene)-R^{Za1}, and R^{Za1} is OH, —NH(CH₃), —N(CH₃)₂, and 5-6 membered heterocyclyl optionally substituted with one or more groups independently selected from F and CH₃. In other embodiments, R^{Ya} is —(C₁-C₂ alkylene)-R^{Za1}, and R^{Za1} is OH. In other embodiments, R^{Ya} is —(C₁-C₂ alkylene)-R^{Za1}, and R^{Za1} is-NH(CH₃). In other embodiments, R^{Ya} is —(C₁-C₂ alkylene)-R^{Za1}, and R^{Za1} is —N(CH₃)₂. In other embodiments, R^{Ya} is —(C₁-C₂ alkylene)-R^{Za1}, and R^{Za1} is 5-6 membered heterocyclyl optionally substituted with one or more groups independently selected from F and CH₃. In other embodiments, R^{Ya} is —(C₁-C₂ alkylene)-R^{Za1}, and R^{Za1} is an unsubstituted 5-6 membered heterocyclyl. In other embodiments, R^{Ya} is —(C₁-C₂ alkylene)-R^{Za1}, and R^{Za1} is 5-6 membered heterocyclyl substituted with one or more groups independently selected from F and CH₃. In other embodiments, R^{Ya} is —(C₁-C₂ alkylene)-R^{Za1}, and R^{Za1} is 5-6 membered heterocyclyl substituted with one CH₃. In other embodiments, R^{Ya} is —(C₁-C₂ alkylene)-R^{Za1}, and R^{Za1} is 5-6 membered heterocyclyl substituted with two F. In other embodiments, R^{Ya} is —(C₁-C₂ alkylene)-R^{Za1}, and R^{Za1} is 5-6 membered heterocyclyl substituted with one CH₃ and two F. In other embodiments, R^{Ya} is 4-6 membered heterocyclyl optionally substituted with one or more groups independently selected from CH₃, —OCH₃, and —OCH₂CH₃. In other embodiments, R^{Ya} is 4-6 membered heterocyclyl optionally substituted with one CH₃. In other embodiments, R^{Ya} is 4-6 membered heterocyclyl optionally substituted with one-OCH₃. In other embodiments, R^{Ya} is 4-6 membered heterocyclyl optionally substituted with one CH₃ and one-OCH₃. In other embodiments, R^{Ya} is 4-6 membered heterocyclyl optionally substituted with one CH₃ and one OCH₂CH₃.

In some embodiments, the invention relates to a compound of any one of formulas (I), (I-A), (I-A-1), (I-A-2), (I-A-3), (I-B), (I-B-1), (I-B-2), (I-B-3), (I-C), (I-C-1), (I-C-2), and (I-C-3), or a pharmaceutically acceptable salt thereof, wherein R is NR^{Xa}R^{Ya}, R^{Xa} and R^{Ya}, together with the nitrogen atom to which they are attached, form a 5-9 membered heterocyclyl optionally substituted with one or more R^{Za2}, and each R^{Za2} is independently selected from halo, OH, C₁-C₆ alkyl, C₁-C₆ alkoxy, NH₂, —NH(C₁-C₆ alkyl), —N(C₁-C₆ alkyl)₂, and —(C₁-C₆ alkylene)-(C₁-C₆ alkoxy). In other embodiments, R is NR^{Xa}R^{Ya}, R^{Xa} and R^{Ya}, together with the nitrogen atom to which they are attached, form a 5-membered heterocyclyl optionally substituted with one or more R^{Za2}. In other embodiments, R is NR^{Xa}R^{Ya}, R^{Xa} and R^{Ya}, together with the nitrogen atom to which they are attached, form a 6-membered heterocyclyl optionally substituted with one or more R^{Za2}. In other embodiments, R is NR^{Xa}R^{Ya}, R^{Xa} and R^{Ya}, together with the nitrogen atom to which they are attached, form a 7-membered heterocyclyl optionally substituted with one or more R^{Za2}. In other embodiments, R is NR^{Xa}R^{Ya}, R^{Xa} and R^{Ya}, together with the nitrogen atom to which they are attached, form an 8-membered heterocyclyl optionally substituted with one or more R^{Za2}. In other embodiments, R is NR^{Xa}R^{Ya}, R^{Xa} and R^{Ya}, together with the nitrogen atom to which they are attached, form a 9-membered heterocyclyl optionally substituted with one or more R^{Za2}. In other embodiments, R^{Xa} and R^{Ya}, together with the nitrogen atom to which they are attached, form an unsubstituted 5-9 membered heterocyclyl. In other embodiments, R^{Xa} and R^{Ya}, together with the nitrogen atom to which they are attached, form a 5-9 membered heterocyclyl optionally substituted with one R^{Za2}. In other embodiments, R^{Xa} and R^{Ya}, together with the nitrogen atom to which they are attached, form a 5-9 membered heterocyclyl optionally substituted with two R^{Za2}. In other embodiments, each R^{Za2} is independently selected from halo, OH, C₁-C₆ alkyl, C₁-C₆ alkoxy, NH₂, —NH(C₁-C₆ alkyl), —N(C₁-C₆ alkyl)₂, and —(C₁-C₆ alkylene)-(C₁-C₆ alkoxy). In other embodiments, at least one R^{Za2} is halo. In other embodiments, at least one R^{Za2} is OH. In other embodiments, at least one R^{Za2} is C₁-C₆ alkyl. In other embodiments, at least one R^{Za2} is C₁-C₆ alkoxy. In other embodiments, at least one R^{Za2} is NH₂. In other embodiments, at least one R^{Za2} is-NH(C₁-C₆ alkyl). In other embodiments, at least one R^{Za2} is-N(C₁-C₆ alkyl)₂. In other embodiments, at least one R^{Za2} is —(C₁-C₆ alkylene)-(C₁-C₆ alkoxy). In other embodiments, each R^{Za2} is independently selected from F, OH, CH₃, —OCH₃, NH₂, —NH(CH₃), —N(CH₃)₂, and —CH₂OCH₃. In other embodiments, at least one R^{Za2} is F. In other embodiments, at least one R^{Za2} is CH₃. In other embodiments, at least one R^{Za2} is —OCH₃. In other embodiments, at least one R^{Za2} is —NH(CH₃). In other embodiments, at least one R^{Za2} is-N(CH₃)₂. In other embodiments, at least one R^{Za2} is-CH₂OCH₃.

In some embodiments, the invention relates to a compound of any one of formulas (I-A-4), (I-B-4), and (I-C-4), or a pharmaceutically acceptable salt thereof, wherein R^{Xa} and R^{Ya}, together with the nitrogen atom to which they are attached, form a 5-9 membered heterocyclyl optionally substituted with one or more R^{Za2}, and each R^{Za2} is independently selected from halo, OH, C₁-C₆ alkyl, C₁-C₆ alkoxy, NH₂, —NH(C₁-C₆ alkyl), —N(C₁-C₆ alkyl)₂, and —(C₁-C₆ alkylene)-(C₁-C₆ alkoxy). In other embodiments, R^{Xa} and R^{Ya}, together with the nitrogen atom to which they are attached, form a 5-membered heterocyclyl optionally substituted with one or more R^{Za2}. In other embodiments, R^{Xa} and R^{Ya}, together with the nitrogen atom to which they are attached, form a 6-membered heterocyclyl optionally substituted with one or more R^{Za2}. In other embodiments, R^{Xa} and R^{Ya}, together with the nitrogen atom to which they are attached, form a 7-membered heterocyclyl optionally substituted with one or more R^{Za2}. In other embodiments, R^{Xa} and R^{Ya}, together with the nitrogen atom to which they are attached, form an 8-membered heterocyclyl optionally substituted with one or more R^{Za2}. In other embodiments, R^{Xa} and R^{Ya}, together with the nitrogen atom to which they are attached, form a 9-membered heterocyclyl optionally substituted with one or more R^{Za2}. In other embodiments, R^{Xa} and R^{Ya}, together with the nitrogen atom to which they are attached, form an unsubstituted 5-9 membered heterocyclyl. In other embodiments, R^{Xa} and R^{Ya}, together with the nitrogen atom to which they are attached, form a 5-9 membered heterocyclyl optionally substituted with one R^{Za2}. In other embodiments, R^{Xa} and R^{Ya}, together with the nitrogen atom to which they are attached, form a 5-9 membered heterocyclyl optionally substituted with two R^{Za2}. In other embodiments, each R^{Za2} is independently selected from halo, OH, C₁-C₆ alkyl, C₁-C₆ alkoxy, NH₂, —NH(C₁-C₆ alkyl), —N(C₁-C₆ alkyl)₂, and —(C₁-C₆ alkylene)-(C₁-C₆ alkoxy). In other embodiments, at least one R^{Za2} is halo. In other embodiments, at least one R^{Za2} is OH. In other embodiments, at least one R^{Za2} is C₁-C₆ alkyl. In other embodiments, at least one R^{Za2} is C₁-C₆ alkoxy. In other embodiments, at least one R^{Za2} is NH₂. In other embodiments, at least one R^{Za2} is-NH(C₁-C₆ alkyl). In other embodiments, at least one R^{Za2} is-N(C₁-C₆ alkyl)₂. In other embodiments, at least one R^{Za2} is —(C₁-C₆ alkylene)-(C₁-C₆ alkoxy). In other embodiments, each R^{Za2} is independently selected from F, OH, CH₃, —OCH₃, NH₂, —NH(CH₃), —N(CH₃)₂, and —CH₂OCH₃. In other embodiments, at least one R^{Za2} is F. In other embodiments, at least one R^{Za2} is CH₃. In other embodiments, at least one R^{Za2} is —OCH₃. In other embodiments, at least one R^{Za2} is-NH(CH₃). In other embodiments, at least one R^{Za2} is-N(CH₃)₂. In other embodiments, at least one R^{Za2} is-CH₂OCH₃.

In some embodiments, the invention relates to a compound of any one of formulas (I), (I-A), (I-A-1), (I-A-2), (I-B), (I-B-1), (I-B-2), (I-C), (I-C-1), and (I-C-2), or a pharmaceutically acceptable salt thereof, wherein R^{4b1} is H or C₁-C₆ alkyl. In other embodiments, R^{4b1} is H or CH₃. In other embodiments, R^{4b1} is H. In other embodiments, R^{4b1} is C₁-C₆ alkyl. In other embodiments, R^{4b1} is CH₃.

In some embodiments, the invention relates to a compound of any one of formulas (I), (I-A), (I-A-1), (I-A-2), (I-B), (I-B-1), (I-B-2), (I-C), (I-C-1), and (I-C-2), or a pharmaceutically acceptable salt thereof, wherein R^{4b2} is H or C₁-C₆ alkyl. In other embodiments, R^{4b2} is H or CH₃. In other embodiments, R^{4b1} is H. In other embodiments, R^{4b2} is C₁-C₆ alkyl. In other embodiments, R^{4b2} is CH₃.

In some embodiments, the invention relates to a compound of any one of formulas (I), (I-A), (I-A-1), (I-A-2), (I-A-3), (I-A-4), (I-B), (I-B-1), (I-B-2), (I-B-3), (I-B-4), (I-C), (I-C-1), (I-C-2), (I-C-3), and (I-C-4), or a pharmaceutically acceptable salt thereof, wherein R^{5b1} is C₁-C₆ alkyl, C₁-C₆ haloalkyl, or —(C₁-C₆ alkylene)-(C₁-C₆ alkoxy). In other embodiments, R^{5b1} is CH₃, CF₃, —CH₂OCH₃, or —CH₂CH₂OCH₃. In other embodiments, R^{5b1} is C₁-C₆ alkyl. In other embodiments, R^{5b1} is CH₃. In other embodiments, R^{5b1} is C₁-C₆ haloalkyl. In other embodiments, R^{5b1} is CF₃. In other embodiments, R^{5b1} is —(C₁-C₆ alkylene)-(C₁-C₆ alkoxy). In other embodiments, R^{5b1} is-CH₂OCH₃. In other embodiments, R^{5b1} is-CH₂CH₂OCH₃.

In some embodiments, the invention relates to a compound of any one of formulas (I), (I-A), (I-A-1), (I-A-2), (I-A-3), (I-A-4), (I-B), (I-B-1), (I-B-2), (I-B-3), (I-B-4), (I-C), (I-C-1), (I-C-2), (I-C-3), and (I-C-4), or a pharmaceutically acceptable salt thereof, wherein R^{5b2} is C₁-C₆ alkyl, C₁-C₆ haloalkyl, or —(C₁-C₆ alkylene)-(C₁-C₆ alkoxy). In other embodiments, R^{5b2} is CH₃, CF₃, —CH₂OCH₃, or —CH₂CH₂OCH₃. In other embodiments, R^{5b2} is C₁-C₆ alkyl. In other embodiments, R⁵⁶² is CH₃. In other embodiments, R^{5b2} is C₁-C₆ haloalkyl. In other embodiments, R^{5b2} is CF₃. In other embodiments, R^{5b2} is —(C₁-C₆ alkylene)-(C₁-C₆ alkoxy). In other embodiments, R^{5b2} is-CH₂OCH₃. In other embodiments, R^{5b2} is-CH₂CH₂OCH₃.

In some embodiments, the invention relates to a compound of any one of formulas (I), (I-A), (I-A-1), (I-A-2), (I-A-3), (I-A-4), (I-B), (I-B-1), (I-B-2), (I-B-3), (I-B-4), (I-C), (I-C-1), (I-C-2), (I-C-3), and (I-C-4), or a pharmaceutically acceptable salt thereof, wherein R^{5b1} and R^{5b2}, together with the carbon atom to which they are attached, form a 4-membered heterocyclyl. In other embodiments, the 4-membered heterocyclyl is an oxetanyl.

In some embodiments, the invention relates to a compound of any one of formulas (I), (I-A), (I-A-1), (I-B), (I-B-1), (I-C), and (I-C-1), or a pharmaceutically acceptable salt thereof, wherein X^{3c} is N or C—R^{3c}, and R^{3c} is H, halo, C₁-C₆ alkyl, C₁-C₆ haloalkyl, —(C₁-C₆ alkylene)-OH, or —(C₁-C₆ alkylene)-(C₁-C₆ alkoxy). In other embodiments, X^{3c} is N. In other embodiments, X^{3c} is C—R^{3c}. In other embodiments, X^{3c} is C—R^{3c}, and R^{3c} is H, halo, C₁-C₆ alkyl, C₁-C₆ haloalkyl, —(C₁-C₆ alkylene)-OH, or —(C₁-C₆ alkylene)-(C₁-C₆ alkoxy). In other embodiments, X^{3c} is C—R^{3c}, and R^{3c} is H. In other embodiments, X^{3c} is C—R^{3c}, and R^{3c} is halo. In other embodiments, X^{3c} is C—R^{3c}, and R^{3c} is C₁-C₆ alkyl. In other embodiments, X^{3c} is C—R^{3c}, and R^{3c} is C₁-C₆ haloalkyl. In other embodiments, X^{3c} is C—R^{3c}, and R^{3c} is —(C₁-C₆ alkylene)-OH. In other embodiments, X^{3c} is C—R^{3c}, and R^{3c} is —(C₁-C₆ alkylene)-(C₁-C₆ alkoxy). In other embodiments, X^{3c} is C—R^{3c}, and R^{3c} is H, F, Cl, CH₃, CF₃, —CH₂OH, or —CH₂OCH₃. In other embodiments, X^{3c} is C—R^{3c}, and R^{3c} is F. In other embodiments, X^{3c} is C—R^{3c}, and R^{3c} is C₁. In other embodiments, X^{3c} is C—R^{3c}, and R^{3c} is CH₃. In other embodiments, X^{3c} is C—R^{3c}, and R^{3c} is CF₃. In other embodiments, X^{3c} is C—R^{3c}, and R^{3c} is-CH₂OH. In other embodiments, X^{3c} is C—R^{3c}, and R^{3c} is —CH₂OCH₃.

In some embodiments, the invention relates to a compound of any one of formulas (I-A-2), (I-A-3), (I-A-4), (I-B-2), (I-B-3), (I-B-4), (I-C-2), (I-C-3), and (I-C-4), or a pharmaceutically acceptable salt thereof, wherein R^{3c} is H, halo, C₁-C₆ alkyl, C₁-C₆ haloalkyl, —(C₁-C₆ alkylene)-OH, or —(C₁-C₆ alkylene)-(C₁-C₆ alkoxy). In other embodiments, R^{3c} is H. In other embodiments, R^{3c} is halo. In other embodiments, R^{3c} is C₁-C₆ alkyl. In other embodiments, R^{3c} is C₁-C₆ haloalkyl. In other embodiments, R^{3c} is —(C₁-C₆ alkylene)-OH. In other embodiments, R^{3c} is —(C₁-C₆ alkylene)-(C₁-C₆ alkoxy). In other embodiments, R^{3c} is H, F, Cl, CH₃, CF₃, —CH₂OH, or —CH₂OCH₃. In other embodiments, R^{3c} is F. In other embodiments, R^{3c} is C₁. In other embodiments, R^{3c} is CH₃. In other embodiments, R^{3c} is CF₃. In other embodiments, R^{3c} is-CH₂OH. In other embodiments, R^{3c} is-CH₂OCH₃.

In some embodiments, the invention relates to a compound of any one of formulas (I), (I-A), (I-B), and (I-C), or a pharmaceutically acceptable salt thereof, wherein X^{4c} is C—R^{4c}, R^{4c} is H, halo, OH, —OBn, C₁-C₆ alkoxy, C₁-C₆ haloalkyl, C₁-C₆ haloalkoxy, or -L¹-L²-(C₃-C₆ cycloalkyl), wherein said cycloalkyl is optionally substituted with 1-2 halo, L¹ is O, and L² is a bond or C₁-C₆ alkylene. In other embodiments, X^{4c} is C—R^{4c}, and R^{4c} is H. In other embodiments, X^{4c} is C—R^{4c}, and R^{4c} is halo. In other embodiments, X^{4c} is C—R^{4c}, and R^{4c} is OH. In other embodiments, X^{4c} is C—R^{4c}, and R^{4c} is —OBn. In other embodiments, X⁴ is C—R^{4c}, and R^{4c} is C₁-C₆ alkoxy. In other embodiments, X^{4c} is C—R^{4c}, and R⁴ is C₁-C₆ haloalkyl. In other embodiments, X⁴ is C—R⁴, and R⁴ is C₁-C₆ haloalkoxy. In other embodiments, X⁴ is C—R^{4c}, R⁴ is -L¹-L²-(C₃-C₆ cycloalkyl), wherein said cycloalkyl is optionally substituted with 1-2 halo; L¹ is O, and L² is a bond or C₁-C₆ alkylene. In other embodiments, X^{4c} is C—R^{4c}, R^{4c} is -L¹-L²-(C₃-C₆ cycloalkyl), wherein said cycloalkyl is optionally substituted with 1-2 halo, L¹ is O, and L² is a bond. In other embodiments, X^{4c} is C—R^{4c}, R^{4c} is -L¹-L²-(C₃-C₆ cycloalkyl), wherein said cycloalkyl is optionally substituted with 1-2 halo, L¹ is O, and L² is C₁-C₆ alkylene. In other embodiments, X^{4c} is C—R^{4c}, and R^{4c} is H, F, OH, —OBn, —OCH₃, —OCH₂CH₃, CHF₂, —OCHF₂, —OCF₃, —O—CH₂-(cyclopropyl), or —O-(cyclobutyl), wherein said cyclobutyl is substituted with 2 F. In other embodiments, X^{4c} is C—R^{4c}, and R^{4c} is F. In other embodiments, X^{4c} is C—R^{4c}, and R⁴ is —OCH₃. In other embodiments, X^{4c} is C—R^{4c}, and R^{4c} is —OCH₂CH₃. In other embodiments, X^{4c} is C—R^{4c}, and R^{4c} is CHF₂. In other embodiments, X^{4c} is C—R^{4c}, and R^{4c} is —OCHF₂. In other embodiments, X^{4c} is C—R^{4c}, and R^{4c} is —OCF₃. In other embodiments, X^{4c} is C—R^{4c}, and R^{4c} is —O—CH₂-(cyclopropyl). In other embodiments, X^{4c} is C—R^{4c}, and R⁴ is —O-(cyclobutyl), wherein said cyclobutyl is substituted with 2 F.

In some embodiments, the invention relates to a compound of any one of formulas (I-A-1), (I-A-2), (I-A-3), (I-A-4), (I-B-1), (I-B-2), (I-B-3), (I-B-4), (I-C-1), (I-C-2), (I-C-3), and (I-C-4), wherein R^{4c} is H, halo, OH, —OBn, C₁-C₆ alkoxy, C₁-C₆ haloalkyl, C₁-C₆ haloalkoxy, or -L¹-L²-(C₃-C₆ cycloalkyl), wherein said cycloalkyl is optionally substituted with 1-2 halo, L¹ is O, and L² is a bond or C₁-C₆ alkylene. In other embodiments, R^{4c} is H. In other embodiments, R^{4c} is halo. In other embodiments, R^{4c} is OH. In other embodiments, R^{4c} is —OBn. In other embodiments, R⁴ is C₁-C₆ alkoxy. In other embodiments, R^{4c} is C₁-C₆ haloalkyl. In other embodiments, R⁴ is C₁-C₆ haloalkoxy. In other embodiments, R⁴ is -L¹-L²-(C₃-C₆ cycloalkyl), wherein said cycloalkyl is optionally substituted with 1-2 halo, L¹ is O, and L² is a bond or C₁-C₆ alkylene. In other embodiments, R⁴ is -L¹-L²-(C₃-C₆ cycloalkyl), wherein said cycloalkyl is optionally substituted with 1-2 halo, L¹ is O, and L² is a bond. In other embodiments, R^{4c} is -L¹-L²-(C₃-C₆ cycloalkyl), wherein said cycloalkyl is optionally substituted with 1-2 halo, L¹ is O, and L² is C₁-C₆ alkylene. In other embodiments, R^{4c} is H, F, OH, —OBn, —OCH₃, —OCH₂CH₃, CHF₂, —OCHF₂, —OCF₃, —O—CH₂-(cyclopropyl), or —O-(cyclobutyl), wherein said cyclobutyl is substituted with 2 F. In other embodiments, R^{4c} is H. In other embodiments, R⁴ is F. In other embodiments, R^{4c} is OH. In other embodiments, R^{4c} is —OBn. In other embodiments, R^{4c} is —OCH₃. In other embodiments, R^{4c} is —OCH₂CH₃. In other embodiments, R^{4c} is CHF₂. In other embodiments, R^{4c} is —OCHF₂. In other embodiments, R^{4c} is —OCF₃. In other embodiments, R⁴ is —O—CH₂-(cyclopropyl). In other embodiments, R⁴ is —O-(cyclobutyl), wherein said cyclobutyl is substituted with 2 F.

In some embodiments, the invention relates to a compound of any one of formulas (I), (I-A), (I-B), and (I-C), or a pharmaceutically acceptable salt thereof, wherein X^{5c} is C—R^{5c}, and R^{5c} is H, halo, OH, —OBn, or -L¹-L²-(C₃-C₆ cycloalkyl), wherein said cycloalkyl is optionally substituted with 1-2 halo, L¹ is O, and L² is a bond. In other embodiments, X^{5c} is C—R^{5c}, and R^{5c} is H. In other embodiments, X^{5c} is C—R^{5c}, and R^{5c} is halo. In other embodiments, X^{5c} is C—R^{5c}, and R^{5c} is OH. In other embodiments, X^{5c} is C—R^{5c}, and R^{5c} is —OBn. In other embodiments, X^{5c} is C—R^{5c}, and R^{5c} is -L¹-L²-(C₃-C₆ cycloalkyl), wherein said cycloalkyl is optionally substituted with 1-2 halo, L¹ is O, and L² is a bond. In other embodiments, X^{5c} is C—R^{5c}, and R^{5c} is H, Cl, OH, —OBn, or —O-(cyclobutyl), wherein said cyclobutyl is substituted with 2 F. In other embodiments, X^{5c} is C—R^{5c}, and R^{5c} is C₁. In other embodiments, X^{5c} is C—R^{5c}, and R^{5c} is —O-(cyclobutyl), wherein said cyclobutyl is substituted with 2 F.

In some embodiments, the invention relates to a compound of any one of formulas (I-A-1), (I-A-2), (I-A-3), (I-A-4), (I-B-1), (I-B-2), (I-B-3), (I-B-4), (I-C-1), (I-C-2), (I-C-3), and (I-C-4), wherein R^{5c} is H, halo, OH, —OBn, or -L¹-L²-(C₃-C₆ cycloalkyl), wherein said cycloalkyl is optionally substituted with 1-2 halo, L¹ is O, and L² is a bond. In other embodiments, R^{5c} is H. In other embodiments, R^{5c} is halo. In other embodiments, R^{5c} is OH. In other embodiments, R^{5c} is —OBn. In other embodiments, R^{5c} is -L¹-L²-(C₃-C₆ cycloalkyl), wherein said cycloalkyl is optionally substituted with 1-2 halo, L¹ is O, and L² is a bond. In other embodiments, R^{5c} is H, Cl, OH, —OBn, or —O-(cyclobutyl), wherein said cyclobutyl is substituted with 2 F. In other embodiments, R^{5c} is C₁. In other embodiments, R^{5c} is —O-(cyclobutyl), wherein said cyclobutyl is substituted with 2 F.

In some embodiments, the invention relates to a compound of any one of formulas (I), (I-A), (I-B), and (I-C), or a pharmaceutically acceptable salt thereof, wherein X⁶⁰ is C—R^{6c}, and R^{6c} is H.

In some embodiments, the invention relates to a compound of any one of formulas (I), (I-A), (I-A-1), (I-A-2), (I-A-3), (I-A-4), (I-B), (I-B-1), (I-B-2), (I-B-3), (I-B-4), (I-C), (I-C-1), (I-C-2), (I-C-3), and (I-C-4), or a pharmaceutically acceptable salt thereof, wherein R^{2c} is OH, halo, C₁-C₆ alkoxy, —(C₁-C₆ alkylene)-(C₁-C₆ alkoxy), —(C₁-C₆ alkylene)-O-(4-6 membered heterocyclyl), —O—(C₂-C₆ alkenylene)-(C₁-C₆ haloalkyl), -L¹-L²-(C₃-C₇ cycloalkyl), or —O-L³-R^{Xc}, wherein said cycloalkyl is optionally substituted with one or more groups independently selected from OH, CN, C₁-C₆ alkyl, C₁-C₆ alkoxy, ═NOH, —C(O) (C₁-C₆ alkyl), and —(C₁-C₆ alkylene)-OH, L¹ is O, L² is a bond or C₁-C₆ alkylene, L³ is a bond, C₁-C₆ alkylene, or C₂-C₆ alkenylene, and R^{Xc} is selected from OH, CN, C₁-C₆ alkoxy, NH₂, —NH(C₁-C₆ alkyl), —N(C₁-C₆ alkyl)₂, —NH(C₁-C₆ haloalkyl), —NH(C₁-C₆ haloalkyl)₂, —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O) (C₁-C₆ alkyl), —C(O) NH₂, —C(O) NH(C₁-C₆ alkyl), —C(O) N(C₁-C₆ alkyl)₂, —NH (4-6 membered heterocyclyl), ═NOH, ═NO(C₁-C₆ alkyl), —N═S(O)(C₁-C₆ alkyl)₂, —C(═NOH)(C₃-C₆ cycloalkyl), 4-8 membered heterocyclyl, and 5-6 membered heteroaryl, wherein said cycloalkyl is optionally substituted with one or more halo, and wherein said heterocyclyl and heteroaryl are optionally substituted with one or more groups independently selected from OH, halo, C₁-C₆ alkyl, C₁-C₆ alkoxy, C₁-C₆ haloalkyl, C₁-C₆ haloalkoxy, and —(C₁-C₆ alkylene)-OH.

In other embodiments, the invention relates to a compound of any one of formulas (I), (I-A), (I-A-1), (I-A-2), (I-A-3), (I-A-4), (I-B), (I-B-1), (I-B-2), (I-B-3), (I-B-4), (I-C), (I-C-1), (I-C-2), (I-C-3), and (I-C-4), or a pharmaceutically acceptable salt thereof, wherein R^{2c} is OH, halo, C₁-C₆ alkoxy, —(C₁-C₆ alkylene)-(C₁-C₆ alkoxy), —(C₁-C₆ alkylene)-O-(4-6 membered heterocyclyl), —O—(C₂-C₆ alkenylene)-(C₁-C₆ haloalkyl). In other embodiments, R^{2c} is OH. In other embodiments, R^{2c} is halo. In other embodiments, R^{2c} is C₁-C₆ alkoxy. In other embodiments, R^{2c} is —(C₁-C₆ alkylene)-(C₁-C₆ alkoxy). In other embodiments, R^{2c} is —(C₁-C₆ alkylene)-O-(4-6 membered heterocyclyl). In other embodiments, R^{2c} is —O—(C₂-C₆ alkenylene)-(C₁-C₆ haloalkyl). In other embodiments, R^{2c} is OH, Cl, —OCH₃, —CH₂OCH₃, —CH₂—O-(4-membered heterocyclyl), or —O—(C₃-C₄ alkenylene)-CF₃. In other embodiments, R^{2c} is C₁. In other embodiments, R^{2c} is —OCH₃. In other embodiments, R^{2c} is-CH₂OCH₃. In other embodiments, R^{2c} is CH₂—O-(4-membered heterocyclyl). In other embodiments, R^{2c} is —O—(C₃-C₄ alkenylene)-CF₃.

In some embodiments, the invention relates to a compound of any one of formulas (I), (I-A), (I-A-1), (I-A-2), (I-A-3), (I-A-4), (I-B), (I-B-1), (I-B-2), (I-B-3), (I-B-4), (I-C), (I-C-1), (I-C-2), (I-C-3), and (I-C-4), or a pharmaceutically acceptable salt thereof, wherein R^{2c} is -L¹-L²-(C₃-C₇ cycloalkyl), L¹ is O, L² is a bond or C₁-C₆ alkylene, and wherein said cycloalkyl is substituted with one or more groups independently selected from OH, CN, —OCH₃, CH₃, ═NOH, —C(O)(CH₃), and —CH₂OH. In other embodiments, R^{2c} is -L¹-L²-(C₃-C₇ cycloalkyl), L¹ is O, L² is a bond, and wherein said cycloalkyl is substituted with one or more groups independently selected from OH, CN, —OCH₃, CH₃, ═NOH, —C(O)(CH₃), and —CH₂OH. R^{2c} is -L¹-L²-(C₃-C₇ cycloalkyl), L¹ is O, L² is C₁-C₆ alkylene, and wherein said cycloalkyl is substituted with one or more groups independently selected from OH, CN, —OCH₃, CH₃, ═NOH, —C(O)(CH₃), and —CH₂OH. In other embodiments, said cycloalkyl is substituted with at least one OH. In other embodiments, said cycloalkyl is substituted with at least one CN. In other embodiments, said cycloalkyl is substituted with at least one-OCH₃. In other embodiments, said cycloalkyl is substituted with at least one CH₃. In other embodiments, said cycloalkyl is substituted with at least one ═NOH. In other embodiments, said cycloalkyl is substituted with at least one —C(O)(CH₃). In other embodiments, said cycloalkyl is substituted with at least one-CH₂OH.

In some embodiments, the invention relates to a compound of any one of formulas (I), (I-A), (I-A-1), (I-A-2), (I-A-3), (I-A-4), (I-B), (I-B-1), (I-B-2), (I-B-3), (I-B-4), (I-C), (I-C-1), (I-C-2), (I-C-3), and (I-C-4), or a pharmaceutically acceptable salt thereof, wherein R^{2c} is —O-L³-R^{Xc}, L³ is a bond, C₁-C₆ alkylene, or C₄-C₅ alkenylene, and R^{Xc} is selected from OH, CN, C₁-C₆ alkoxy, NH₂, —NH(C₁-C₆ alkyl), —N(C₁-C₆ alkyl)₂, —NH(C₁-C₆ haloalkyl), —NH(C₁-C₆ haloalkyl)₂, —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O) (C₁-C₆ alkyl), —C(O) NH₂, —C(O) NH(C₁-C₆ alkyl), —C(O) N(C₁-C₆ alkyl)₂, —NH (4-6 membered heterocyclyl), ═NOH, ═NO(C₁-C₆ alkyl), —N═S(O)(C₁-C₆ alkyl)₂, C(═NOH)(C₃-C₆ cycloalkyl), 4-8 membered heterocyclyl, and 5-6 membered heteroaryl, wherein said cycloalkyl is optionally substituted with one or more halo, and wherein said heterocyclyl and heteroaryl are optionally substituted with one or more groups independently selected from OH, halo, C₁-C₆ alkyl, C₁-C₆ alkoxy, C₁-C₆ haloalkyl, C₁-C₆ haloalkoxy, and —(C₁-C₆ alkylene)-OH. In other embodiments, R^{2c} is —O-L³-R^{Xc}, and L³ is a bond. In other embodiments, R^{2c} is —O-L³-R^{Xc}, and L³ is C₁-C₆ alkylene. In other embodiments, R^{2c} is —O-L³-R^{Xc}, and C₄-C₅ alkenylene. In other embodiments, R^{2c} is —O-L³-R^{Xc}, and R^{Xc} is OH. In other embodiments, R^{2c} is —O-L³-R^{Xc}, and R^{Xc} is CN. In other embodiments, R^{2c} is —O-L³-R^{Xc}, and R^{Xc} is C₁-C₆ alkoxy. In other embodiments, R^{2c} is —O-L³-R^{Xc}, and R^{Xc} is NH₂. In other embodiments, R^{2c} is —O-L³-R^{Xc}, and R^{Xc} is-NH(C₁-C₆ alkyl). In other embodiments, R^{2c} is —O-L³-R^{Xc}, and R^{Xc} is —N(C₁-C₆ alkyl)₂. In other embodiments, R^{2c} is —O-L³-R^{Xc}, and R^{Xc} is-NH(C₁-C₆ haloalkyl). In other embodiments, R^{2c} is —O-L³-R^{Xc}, and R^{Xc} is-N(C₁-C₆ haloalkyl)₂. In other embodiments, R^{2c} is —O-L³-R^{Xc}, and R^{Xc} is-CH(CH₂OH)₂. In other embodiments, R^{2c} is —O-L³-R^{Xc}, and R^{Xc} is —CH(CH₂OH) (CH₂OCH₃). In other embodiments, R^{2c} is —O-L³-R^{Xc}, and R^{Xc} is-CH(CH₂OH) (OCH₃). In other embodiments, R^{2c} is —O-L³-R^{Xc}, and R^{Xc} is-CH(CH₂OCH₃) (OCH₃). In other embodiments, R^{2c} is —O-L³-R^{Xc}, and R^{Xc} is-CH(CH₂OH)(CF₃). In other embodiments, R^{2c} is —O-L³-R^{Xc}, and R^{Xc} is —C(O) (C₁-C₆ alkyl). In other embodiments, R^{2c} is —O-L³-R^{Xc}, and R^{Xc} is —C(O) NH₂. In other embodiments, R^{2c} is —O-L³-R^{Xc}, and R^{Xc} is —C(O) NH(C₁-C₆ alkyl). In other embodiments, R^{2c} is —O-L³-R^{Xc}, and R^{Xc} is —C(O) N(C₁-C₆ alkyl)₂. In other embodiments, R^{2c} is —O-L³-R^{Xc}, and R^{Xc} is —NH (4-6 membered heterocyclyl). In other embodiments, R^{2c} is —O-L³-R^{Xc}, and R^{Xc} is ═NOH. In other embodiments, R^{2c} is —O-L³-R^{Xc}, and R^{Xc} is ═NO(C₁-C₆ alkyl). In other embodiments, R^{2c} is —O-L³-R^{Xc}, and R^{Xc} is-N═S(O)(C₁-C₆ alkyl)₂. In other embodiments, R^{2c} is —O-L³-R^{Xc}, and R^{Xc} is —C(═NOH)(C₃-C₆ cycloalkyl), where said cycloalkyl is optionally substituted with one or more halo. In other embodiments, R^{2c} is —O-L³-R^{Xc}, and R^{Xc} is 4-8 membered heterocyclyl, wherein said heterocyclyl is optionally substituted with one or more groups independently selected from OH, halo, C₁-C₆ alkyl, C₁-C₆ alkoxy, C₁-C₆ haloalkyl, C₁-C₆ haloalkoxy, and —(C₁-C₆ alkylene)-OH. In other embodiments, R^{2c} is —O-L³-R^{Xc}, and R^{Xc} is and 5-6 membered heteroaryl, wherein said heteroaryl is optionally substituted with one or more groups independently selected from OH, halo, C₁-C₆ alkyl, C₁-C₆ alkoxy, C₁-C₆ haloalkyl, C₁-C₆ haloalkoxy, and —(C₁-C₆ alkylene)-OH. In other embodiments, R^{2c} is —O-L³-R^{Xc}, and R^{Xc} is selected from OH, CN, —OCH₃, —NH(CH₃), —NH(CH(CH₃)₂), —N(CH₃)₂, —NH(CH₂CHF₂), —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O)(CH₃), —C(O) NH(CH₃), —NH (4-5 membered heterocyclyl), ═NOH, ═NO(CH₃), —N═S(O)(CH₃)₂, —C(═NOH)(C₃-C₄ cycloalkyl), 4-8 membered heterocyclyl optionally substituted with one or more groups independently selected from OH, F, CH₃, —OCH₃, CHF₂, CF₃, —OCHF₂, and —CH₂OH, and 5-membered heteroaryl optionally substituted with CH₃, and wherein said cycloalkyl is optionally substituted with one F. In other embodiments, R^{2c} is —O-L³-R^{Xc}, and R^{Xc}—OCH₃. In other embodiments, R^{2c} is —O-L³-R^{Xc}, and R^{Xc} is-NH(CH₃). In other embodiments, R^{2c} is —O-L³-R^{Xc}, and R^{Xc} is-NH(CH(CH₃)₂). In other embodiments, R^{2c} is —O-L³-R^{Xc}, and R^{Xc} is-N(CH₃)₂. In other embodiments, R^{2c} is —O-L³-R^{Xc}, and R^{Xc} is —NH(CH₂CHF₂). In other embodiments, R^{2c} is —O-L³-R^{Xc}, and R^{Xc} is —C(O)(CH₃). In other embodiments, R^{2c} is —O-L³-R^{Xc}, and R^{Xc} is-C(O) NH(CH₃). In other embodiments, R^{2c} is —O-L³-R^{Xc}, and R^{Xc} is —NH (4-5 membered heterocyclyl). In other embodiments, R^{2c} is —O-L³-R^{Xc}, and R^{Xc} is ═NO(CH₃). In other embodiments, R^{2c} is —O-L³-R^{Xc}, and R^{Xc} is-C(═NOH)(C₃-C₄ cycloalkyl), wherein said cycloalkyl is optionally substituted with one F. In other embodiments, R^{2c} is —O-L³-R^{Xc}, and R^{Xc} is 4-8 membered heterocyclyl optionally substituted with one or more groups independently selected from OH, F, CH₃, —OCH₃, CHF₂, CF₃, —OCHF₂, and —CH₂OH. In other embodiments, R^{2c} is —O-L³-R^{Xc}, and R^{Xc} is 5-membered heteroaryl optionally substituted with CH₃.

In some embodiments, the invention relates to a compound of any one of formulas (I), (I-A), (I-A-1), (I-B), (I-B-1), (I-C), and (I-C-1), or the pharmaceutically acceptable salt thereof, wherein X^{3c} is C—R^{3c}, and R^{2c} and R^{3c}, together with the carbon atoms to which they are attached, form a ring of formula:

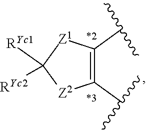

where Z¹ is O or CH₂, Z² is O or CF₂, and R^{Yc1} and R^{Yc2} are each, independently, H or F. In other embodiments, X^{3c} is C—R^{3c}, and R^{2c} and R^{3c}, together with the carbon atoms to which they are attached, form a ring of formula:

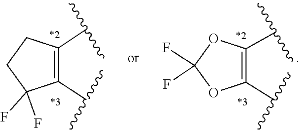 [SMILES: *C1=C(*)C(F)(F)CC1 ; *C1=C(*)OC(F)(F)O1]

In other embodiments, X^{3c} is C—R^{3c}, and R^{2c} and R^{3c}, together with the carbon atoms to which they are attached, form a ring of formula:

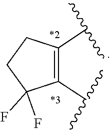 [SMILES: *C1=C(*)C(F)(F)CC1]

In other embodiments, X^{3c} is C—R^{3c}, and R^{2c} and R^{3c}, together with the carbon atoms to which they are attached, form a ring of formula:

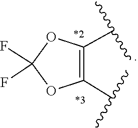 [SMILES: *C1=C(*)OC(F)(F)O1]

In some embodiments, the invention relates to a compound of any one of formulas (I-A-2), (I-A-3), (I-A-4), (I-B-2), (I-B-3), (I-B-4), (I-C-2), (I-C-3), and (I-C-4), or a pharmaceutically acceptable salt thereof, wherein R^{2c} and R^{3c}, together with the carbon atoms to which they are attached, form a ring of formula:

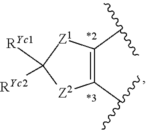

where Z¹ is O or CH₂, Z² is O or CF₂, and R^{Yc1} and RYe2 are each, independently, H or F. In other embodiments, R^{2c} and R^{3c}, together with the carbon atoms to which they are attached, form a ring of formula:

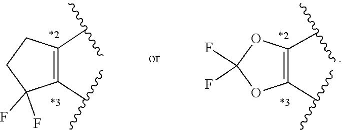 [SMILES: *C1=C(*)C(F)(F)CC1 ; *C1=C(*)OC(F)(F)O1]

In other embodiments, R^{2c} and R^{3c}, together with the carbon atoms to which they are attached, form a ring of formula:

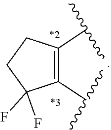 [SMILES: *C1=C(*)C(F)(F)CC1]

In other embodiments, R^{2c} and R^{3c}, together with the carbon atoms to which they are attached, form a ring of formula:

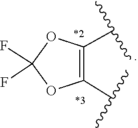 [SMILES: *C1=C(*)OC(F)(F)O1]

In some embodiments, the invention relates to a compound of any one of formulas (I), (I-A), (I-A-1), (I-A-2), (I-A-3), (I-A-4), (I-B), (I-B-1), (I-B-2), (I-B-3), (I-B-4), (I-C), (I-C-1), (I-C-2), (I-C-3), and (I-C-4), or any embodiment thereof, in a salt form. In other embodiments, the compound is a trifluoroacetate salt or a hydrochloride salt. In other embodiments, the compound is a trifluoroacetate salt. In other embodiments, the compound is a hydrochloride salt.

In some embodiments, the invention relates to a compound of any one of formulas (I), (I-A), (I-A-1), (I-A-2), (I-A-3), (I-A-4), (I-B), (I-B-1), (I-B-2), (I-B-3), (I-B-4), (I-C), (I-C-1), (I-C-2), (I-C-3), and (I-C-4), or any embodiment thereof, i.e., the compound in non-salt form.

In some embodiments, the invention relates to a compound selected from Table A, or a pharmaceutically acceptable salt thereof. In other embodiments, the invention relates to a compound selected from Table A, i.e., the compound in non-salt form.

TABLE A
Compound Structures and Names.

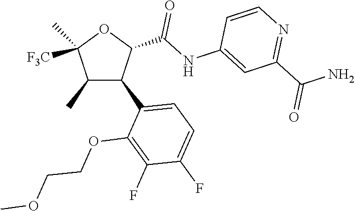 [SMILES: COCCOc1c([C@@H]2[C@@H](C(=O)Nc3ccnc(C(N)=O)c3)O[C@](C)(C(F)(F)F)[C@@H]2C)ccc(F)c1F]

4-((2S,3R,4R,5S)-3-(3,4-difluoro-2-(2-methoxyethoxy)-
phenyl)-4,5-dimethyl-5-(trifluoromethyl)tetrahydrofuran-
2-carboxamido)picolinamide

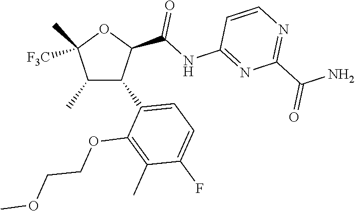 [SMILES: COCCOc1c([C@H]2[C@H](C(=O)Nc3ccnc(C(N)=O)n3)O[C@@](C)(C(F)(F)F)[C@H]2C)ccc(F)c1C]

4-((2R,3S,4S,5R)-3-(4-fluoro-2-(2-methoxyethoxy)-3-
methylphenyl)-4,5-dimethyl-5-(trifluoromethyl)tetra-
hydrofuran-2-carboxamido)pyrimidine-2-carboxamide

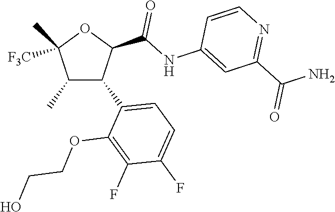 [SMILES: C[C@H]1[C@@H](c2ccc(F)c(F)c2OCCO)[C@H](C(=O)Nc2ccnc(C(N)=O)c2)O[C@@]1(C)C(F)(F)F]

4-((2R,3S,4S,5R)-3-(3,4-difluoro-2-(2-hydroxyethoxy)
phenyl)-4,5-dimethyl-5-(trifluoromethyl)tetrahydro-
furan-2-carboxamido)picolinamide

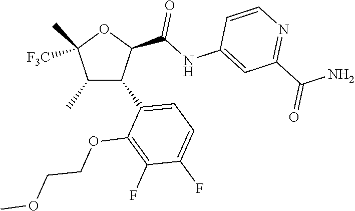 [SMILES: COCCOc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(2-methoxyethoxy)-
phenyl)-4,5-dimethyl-5-(trifluoromethyl)tetrahydrofuran-
2-carboxamido)picolinamide

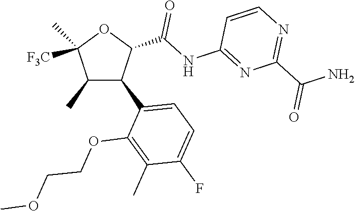 [SMILES: COCCOc1c([C@@H]2[C@@H](C(=O)Nc3ccnc(C(N)=O)n3)O[C@](C)(C(F)(F)F)[C@@H]2C)ccc(F)c1C]

4-((2S,3R,4R,5S)-3-(4-fluoro-2-(2-methoxyethoxy)-3-
methylphenyl)-4,5-dimethyl-5-(trifluoromethyl)tetra-
hydrofuran-2-carboxamido)pyrimidine-2-carboxamide

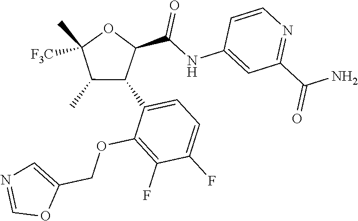 [SMILES: C[C@H]1[C@@H](c2ccc(F)c(F)c2OCc2cnco2)[C@H](C(=O)Nc2ccnc(C(N)=O)c2)O[C@@]1(C)C(F)(F)F]

4-((2R,3S,4S,5R)-3-(3,4-difluoro-2-(oxazol-5-ylmethoxy)-
phenyl)-4,5-dimethyl-5-(trifluoromethyl)tetrahydrofuran-
2-carboxamido)picolinamide

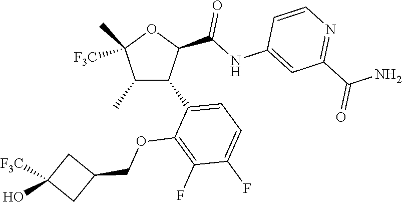 [SMILES: C[C@H]1[C@@H](c2ccc(F)c(F)c2OC[C@H]2C[C@](O)(C(F)(F)F)C2)[C@H](C(=O)Nc2ccnc(C(N)=O)c2)O[C@@]1(C)C(F)(F)F]

4-((2R,3S,4S,5R)-3-(3,4-difluoro-2-(((1s,3R)-3-hydroxy-3-
(trifluoromethyl)cyclobutyl)methoxy)phenyl)-4,5-dimethyl-5-
(trifluoromethyl)tetrahydrofuran-2-carboxamido)picolinamide

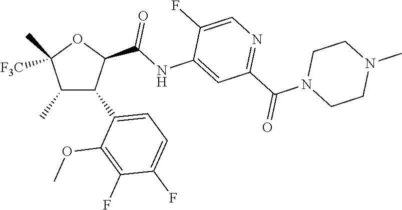 [SMILES: COc1c([C@H]2[C@H](C(=O)Nc3cc(C(=O)N4CCN(C)CC4)ncc3F)O[C@@](C)(C(F)(F)F)[C@H]2C)ccc(F)c1F]

(2R,3S,4S,5R)-3-(3,4-difluoro-2-methoxyphenyl)-N-(5-fluoro-2-
(4-methylpiperazine-1-carbonyl)pyridin-4-yl)-4,5-dimethyl-5-
(trifluoromethyl)tetrahydrofuran-2-carboxamide

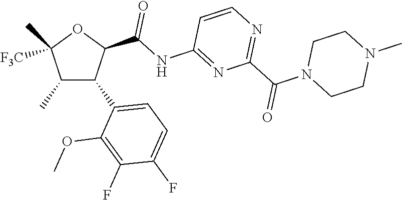 [SMILES: COc1c([C@H]2[C@H](C(=O)Nc3ccnc(C(=O)N4CCN(C)CC4)n3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(2-(4-methylpiperazine-1-carbonyl)pyrimidin-4-yl)-5-(trifluoro-
methyl)tetrahydrofuran-2-carboxamide

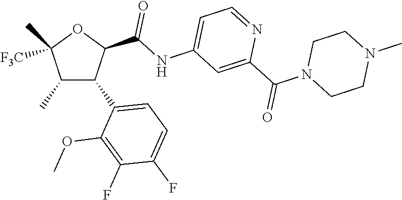 [SMILES: COc1c([C@H]2[C@H](C(=O)Nc3ccnc(C(=O)N4CCN(C)CC4)c3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(2-(4-methylpiperazine-1-carbonyl)pyridin-4-yl)-5-(trifluoro-
methyl)tetrahydrofuran-2-carboxamide

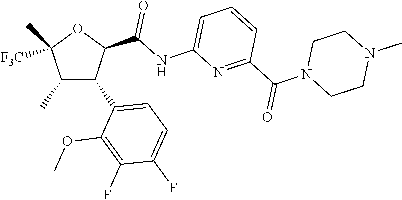 [SMILES: COc1c([C@H]2[C@H](C(=O)Nc3cccc(C(=O)N4CCN(C)CC4)n3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(6-(4-methylpiperazine-1-carbonyl)pyridin-2-yl)-5-(trifluoro-
methyl)tetrahydrofuran-2-carboxamide

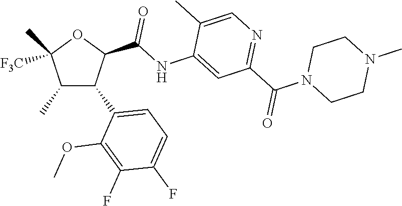 [SMILES: COc1c([C@H]2[C@H](C(=O)Nc3cc(C(=O)N4CCN(C)CC4)ncc3C)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(5-methyl-2-(4-methylpiperazine-1-carbonyl)pyridin-4-yl)-5-
(trifluoromethyl)tetrahydrofuran-2-carboxamide

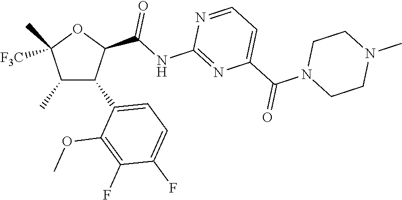 [SMILES: COc1c([C@H]2[C@H](C(=O)Nc3nccc(C(=O)N4CCN(C)CC4)n3)O[C@@](C)(C(F)(F)F)[C@H]2C)ccc(F)c1F]

(2R,3S,4S,5R)-3-(3,4-difluoro-2-methoxyphenyl)-4,5-dimethyl-N-
(4-(4-methylpiperazine-1-carbonyl)pyrimidin-2-yl)-5-(trifluoro-
methyl)tetrahydrofuran-2-carboxamide

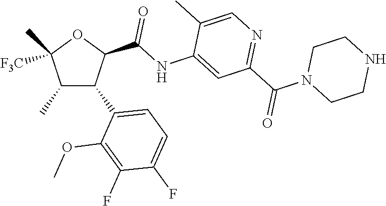 [SMILES: COc1c([C@H]2[C@H](C(=O)Nc3cc(C(=O)N4CCNCC4)ncc3C)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(5-methyl-2-(piperazine-1-carbonyl)pyridin-4-yl)-5-(trifluoro-
methyl)tetrahydrofuran-2-carboxamide

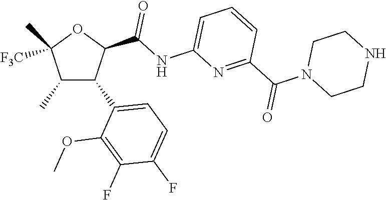 [SMILES: COc1c([C@H]2[C@H](C(=O)Nc3cccc(C(=O)N4CCNCC4)n3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(6-(piperazine-1-carbonyl)pyridin-2-yl)-5-(trifluoromethyl)-
tetrahydrofuran-2-carboxamide

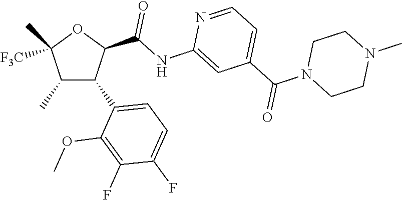 [SMILES: COc1c([C@H]2[C@H](C(=O)Nc3cc(C(=O)N4CCN(C)CC4)ccn3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(4-(4-methylpiperazine-1-carbonyl)pyridin-2-yl)-5-(trifluoro-
methyl)tetrahydrofuran-2-carboxamide

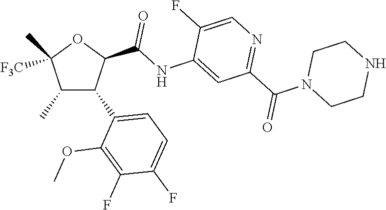 [SMILES: COc1c([C@H]2[C@H](C(=O)Nc3cc(C(=O)N4CCNCC4)ncc3F)O[C@@](C)(C(F)(F)F)[C@H]2C)ccc(F)c1F]

(2R,3S,4S,5R)-3-(3,4-difluoro-2- methoxyphenyl)-N-(5-fluoro-
2-(piperazine-1-carbonyl)pyridin-4-yl)-4,5-dimethyl-5-(tri-
fluoromethyl)tetrahydrofuran-2-carboxamide

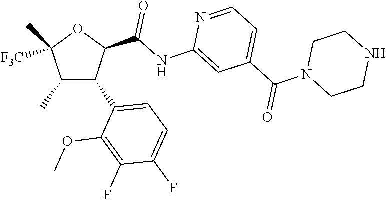 [SMILES: COc1c([C@H]2[C@H](C(=O)Nc3cc(C(=O)N4CCNCC4)ccn3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(4-(piperazine-1-carbonyl)pyridin-2-yl)-5-(trifluoromethyl)-
tetrahydrofuran-2-carboxamide

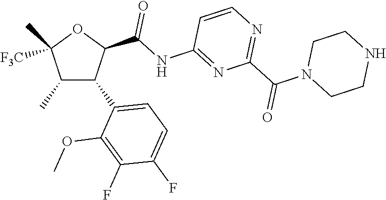 [SMILES: COc1c([C@H]2[C@H](C(=O)Nc3ccnc(C(=O)N4CCNCC4)n3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(2-(piperazine-1-carbonyl)pyrimidin-4-yl)-5-(trifluorometh-
yl)tetrahydrofuran-2-carboxamide

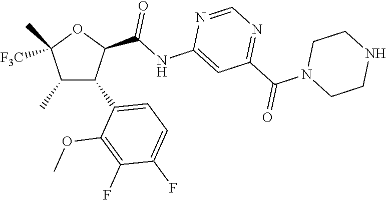 [SMILES: COc1c([C@H]2[C@H](C(=O)Nc3cc(C(=O)N4CCNCC4)ncn3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(6-(piperazine-1-carbonyl)pyrimidin-4-yl)-5-(trifluorometh-
yl)tetrahydrofuran-2-carboxamide

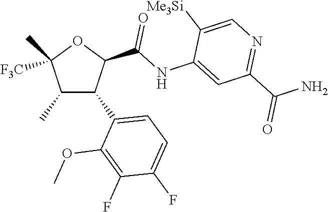 [SMILES: COc1c([C@H]2[C@H](C(=O)Nc3cc(C(N)=O)ncc3[Si](C)(C)C)O[C@@](C)(C(F)(F)F)[C@H]2C)ccc(F)c1F]

4-((2R,3S,4S,5R)-3-(3,4-difluoro-2-methoxyphenyl)-
4,5-dimethyl-5-(trifluoromethyl)tetrahydrofuran-2-
carboxamido)-5-(trimethylsilyl)picolinamide

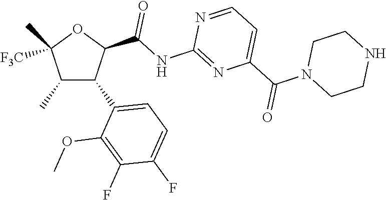 [SMILES: COc1c([C@H]2[C@H](C(=O)Nc3nccc(C(=O)N4CCNCC4)n3)O[C@@](C)(C(F)(F)F)[C@H]2C)ccc(F)c1F]

(2R,3S,4S,5R)-3-(3,4-difluoro-2-methoxyphenyl)-4,5-dimeth-
yl-N-(4-(piperazine-1-carbonyl)pyrimidin-2-yl)-5-(trifluoro-
methyl)tetrahydrofuran-2-carboxamide

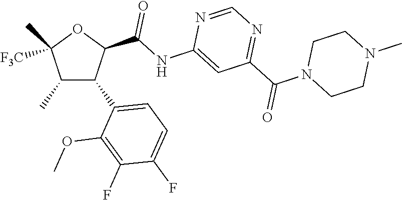 [SMILES: COc1c([C@H]2[C@H](C(=O)Nc3cc(C(=O)N4CCN(C)CC4)ncn3)O[C@@](C)(C(F)(F)F)[C@H]2C)ccc(F)c1F]

(2R,3S,4S,5R)-3-(3,4-difluoro-2-methoxyphenyl)-4,5-dimethyl-N-
(6-(4-methylpiperazine-1-carbonyl)pyrimidin-4-yl)-5-(trifluoro-
methyl)tetrahydrofuran-2-carboxamide

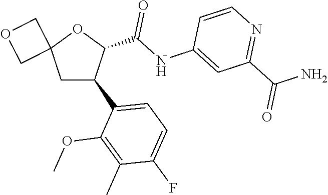 [SMILES: COc1c([C@H]2CC3(COC3)O[C@@H]2C(=O)Nc2ccnc(C(N)=O)c2)ccc(F)c1C]

4-((6S,7R)-7-(4-fluoro-2-methoxy-3-methylphen-
yl)-2,5-dioxaspiro[3.4]octane-6-carboxamido)-
picolinamide

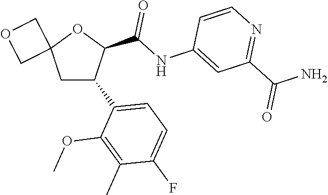 [SMILES: COc1c([C@@H]2CC3(COC3)O[C@H]2C(=O)Nc2ccnc(C(N)=O)c2)ccc(F)c1C]

4-((6R,7S)-7-(4-fluoro-2-methoxy-3-methylphen-
yl)-2,5-dioxaspiro[3.4]octane-6-carboxamido)-
picolinamide

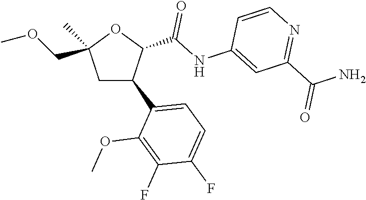 [SMILES: COC[C@]1(C)C[C@H](c2ccc(F)c(F)c2OC)[C@@H](C(=O)Nc2ccnc(C(N)=O)c2)O1]

4-((2S,3R,5S)-3-(3,4-difluoro-2-methoxyphenyl)-5-(meth-
oxymethyl)-5-methyltetrahydrofuran-2-carboxamido)-
picolinamide

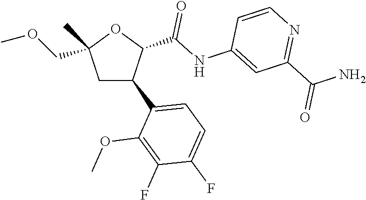 [SMILES: COC[C@@]1(C)C[C@H](c2ccc(F)c(F)c2OC)[C@@H](C(=O)Nc2ccnc(C(N)=O)c2)O1]

4-((2S,3R,5R)-3-(3,4-difluoro-2-methoxyphenyl)-5-(meth-
oxymethyl)-5-methyltetrahydrofuran-2-carboxamido)-
picolinamide

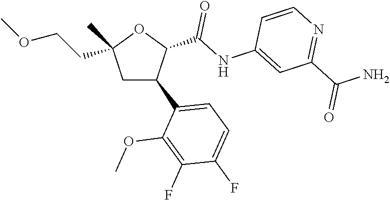 [SMILES: COCC[C@@]1(C)C[C@H](c2ccc(F)c(F)c2OC)[C@@H](C(=O)Nc2ccnc(C(N)=O)c2)O1]

4-((2S,3R,5R)-3-(3,4-difluoro-2-methoxyphenyl)-5-(2-meth-
oxyethyl)-5-methyltetrahydrofuran-2-carboxamido)-
picolinamide

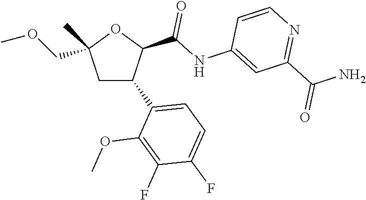 [SMILES: COC[C@@]1(C)C[C@@H](c2ccc(F)c(F)c2OC)[C@H](C(=O)Nc2ccnc(C(N)=O)c2)O1]

4-((2R,3S,5R)-3-(3,4-difluoro-2-methoxyphenyl)-5-(meth-
oxymethyl)-5-methyltetrahydrofuran-2-carboxamido)-
picolinamide

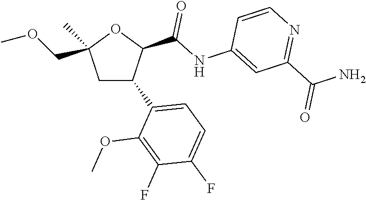 [SMILES: COC[C@]1(C)C[C@@H](c2ccc(F)c(F)c2OC)[C@H](C(=O)Nc2ccnc(C(N)=O)c2)O1]

4-((2R,3S,5S)-3-(3,4-difluoro-2-methoxyphenyl)-5-(meth-
oxymethyl)-5-methyltetrahydrofuran-2-carboxamido)-
picolinamide

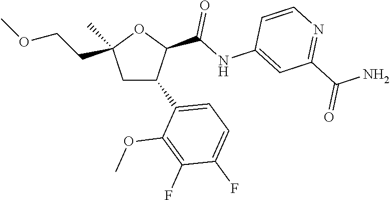 [SMILES: COCC[C@]1(C)C[C@@H](c2ccc(F)c(F)c2OC)[C@H](C(=O)Nc2ccnc(C(N)=O)c2)O1]

4-((2R,3S,5S)-3-(3,4-difluoro-2-methoxyphenyl)-5-(2-meth-
oxyethyl)-5-methyltetrahydrofuran-2-carboxamido)-
picolinamide

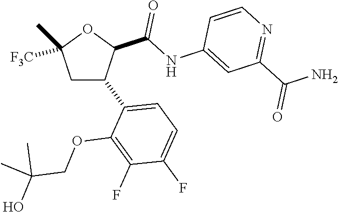 [SMILES: CC(C)(O)COc1c([C@@H]2C[C@](C)(C(F)(F)F)O[C@H]2C(=O)Nc2ccnc(C(N)=O)c2)ccc(F)c1F]

rel-4-((2R,3S,5R)-3-(3,4-difluoro-2-(2-hydroxy-2-
methylpropoxy)phenyl)-5-methyl-5-(trifluorometh-
yl)tetrahydrofuran-2-carboxamido)picolinamide

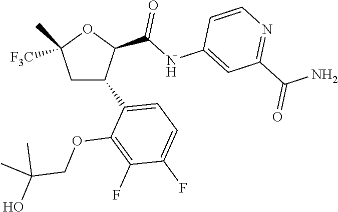 [SMILES: CC(C)(O)COc1c([C@@H]2C[C@](C)(C(F)(F)F)O[C@H]2C(=O)Nc2ccnc(C(N)=O)c2)ccc(F)c1F]

4-((2R,3S,5R)-3-(3,4-difluoro-2-(2-hydroxy-2-methyl-
propoxy)phenyl)-5-methyl-5-(trifluoromethyl)tetra-
hydrofuran-2-carboxamido)picolinamide

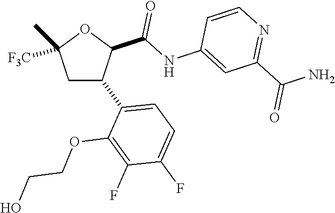 [SMILES: C[C@]1(C(F)(F)F)C[C@@H](c2ccc(F)c(F)c2OCCO)[C@H](C(=O)Nc2ccnc(C(N)=O)c2)O1]

rel-4-((2R,3S,5R)-3-(3,4-difluoro-2-(2-hydroxyeth-
oxy)phenyl)-5-methyl-5-(trifluoromethyl)tetrahydro-
furan-2-carboxamido)picolinamide

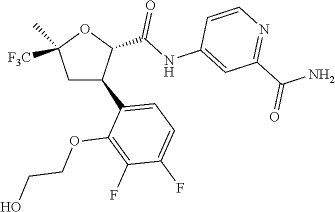 [SMILES: C[C@@]1(C(F)(F)F)C[C@H](c2ccc(F)c(F)c2OCCO)[C@@H](C(=O)Nc2ccnc(C(N)=O)c2)O1]

4-((2S,3R,5S)-3-(3,4-difluoro-2-(2-hydroxyethoxy)-
phenyl)-5-methyl-5-(trifluoromethyl)tetrahydrofuran-
2-carboxamido)picolinamide

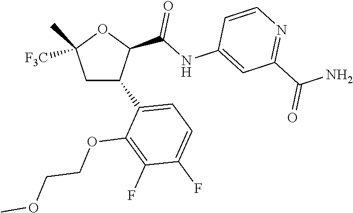 [SMILES: COCCOc1c([C@@H]2C[C@](C)(C(F)(F)F)O[C@H]2C(=O)Nc2ccnc(C(N)=O)c2)ccc(F)c1F]

4-((2R,3S,5R)-3-(3,4-difluoro-2-(2-methoxyethoxy)-
phenyl)-5-methyl-5-(trifluoromethyl)tetrahydrofuran-
2-carboxamido)picolinamide

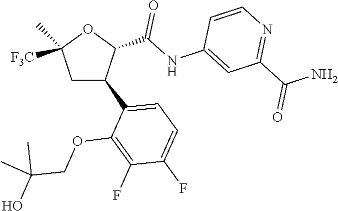 [SMILES: CC(C)(O)COc1c([C@H]2C[C@@](C)(C(F)(F)F)O[C@@H]2C(=O)Nc2ccnc(C(N)=O)c2)ccc(F)c1F]

4-((2S,3R,5S)-3-(3,4-difluoro-2-(2-hydroxy-2-methyl-
propoxy)phenyl)-5-methyl-5-(trifluoromethyl)tetra-
hydrofuran-2-carboxamido)picolinamide

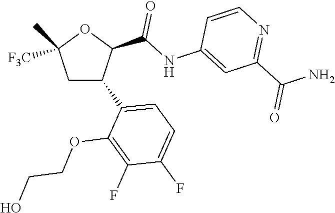 [SMILES: C[C@]1(C(F)(F)F)C[C@@H](c2ccc(F)c(F)c2OCCO)[C@H](C(=O)Nc2ccnc(C(N)=O)c2)O1]

4-((2R,3S,5R)-3-(3,4-difluoro-2-(2-hydroxyethoxy)-
phenyl)-5-methyl-5-(trifluoromethyl)tetrahydrofuran-
2-carboxamido)picolinamide

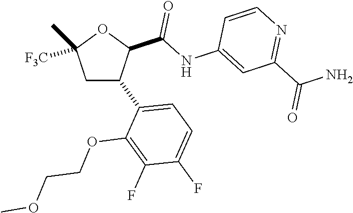 [SMILES: COCCOc1c([C@@H]2C[C@](C)(C(F)(F)F)O[C@H]2C(=O)Nc2ccnc(C(N)=O)c2)ccc(F)c1F]

rel-4-((2R,3S,5R)-3-(3,4-difluoro-2-(2-methoxyethoxy)-
phenyl)-5-methyl-5-(trifluoromethyl)tetrahydrofuran-2-
carboxamido)picolinamide

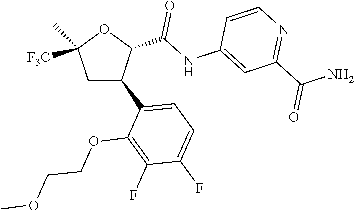 [SMILES: COCCOc1c([C@H]2C[C@@](C)(C(F)(F)F)O[C@@H]2C(=O)Nc2ccnc(C(N)=O)c2)ccc(F)c1F]

4-((2S,3R,5S)-3-(3,4-difluoro-2-(2-methoxyethoxy)-
phenyl)-5-methyl-5-(trifluoromethyl)tetrahydrofuran-
2-carboxamido)picolinamide

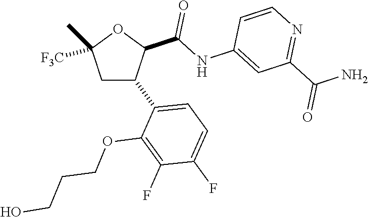 [SMILES: C[C@]1(C(F)(F)F)C[C@@H](c2ccc(F)c(F)c2OCCCO)[C@H](C(=O)Nc2ccnc(C(N)=O)c2)O1]

rel-4-((2R,3S,5R)-3-(3,4-difluoro-2-(3-hydroxypropoxy)-
phenyl)-5-methyl-5-(trifluoromethyl)tetrahydrofuran-2-
carboxamido)picolinamide

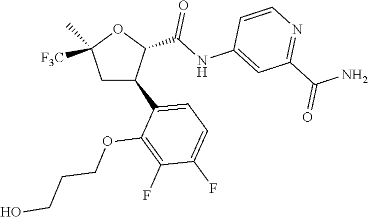 [SMILES: C[C@@]1(C(F)(F)F)C[C@H](c2ccc(F)c(F)c2OCCCO)[C@@H](C(=O)Nc2ccnc(C(N)=O)c2)O1]

4-((2S,3R,5S)-3-(3,4-difluoro-2-(3-hydroxypropoxy)phen-
yl)-5-methyl-5-(trifluoromethyl)tetrahydrofuran-2-carbox-
amido)picolinamide

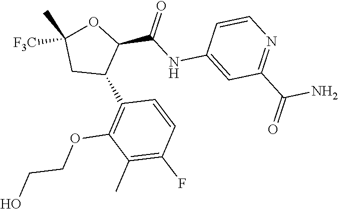 [SMILES: Cc1c(F)ccc([C@@H]2C[C@](C)(C(F)(F)F)O[C@H]2C(=O)Nc2ccnc(C(N)=O)c2)c1OCCO]

4-((2R,3S,5R)-3-(4-fluoro-2-(2-hydroxyethoxy)-3-
methylphenyl)-5-methyl-5-(trifluoromethyl)tetra-
hydrofuran-2-carboxamido)picolinamide

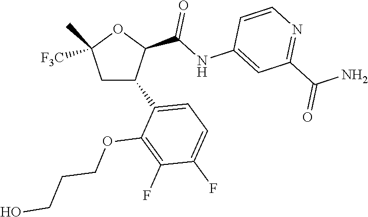 [SMILES: C[C@]1(C(F)(F)F)C[C@@H](c2ccc(F)c(F)c2OCCCO)[C@H](C(=O)Nc2ccnc(C(N)=O)c2)O1]

4-((2R,3S,5R)-3-(3,4-difluoro-2-(3-hydroxypropoxy)phen-
yl)-5-methyl-5-(trifluoromethyl)tetrahydrofuran-2-carbox-
amido)picolinamide

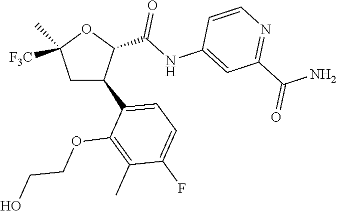 [SMILES: Cc1c(F)ccc([C@H]2C[C@@](C)(C(F)(F)F)O[C@@H]2C(=O)Nc2ccnc(C(N)=O)c2)c1OCCO]

4-((2S,3R,5S)-3-(4-fluoro-2-(2-hydroxyethoxy)-3-
methylphenyl)-5-methyl-5-(trifluoromethyl)tetra-
hydrofuran-2-carboxamido)picolinamide

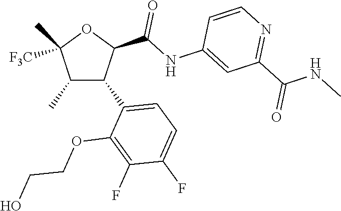 [SMILES: CNC(=O)c1cc(NC(=O)[C@@H]2O[C@@](C)(C(F)(F)F)[C@@H](C)[C@H]2c2ccc(F)c(F)c2OCCO)ccn1]

4-((2R,3S,4S,5R)-3-(3,4-difluoro-2-(2-hydroxyethoxy)-
phenyl)-4,5-dimethyl-5-(trifluoromethyl)tetrahydro-
furan-2-carboxamido)-N-methylpicolinamide

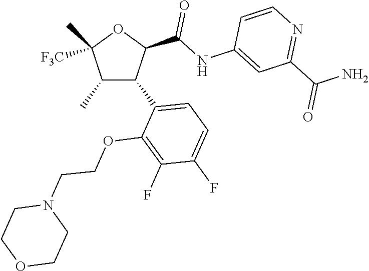 [SMILES: C[C@H]1[C@@H](c2ccc(F)c(F)c2OCCN2CCOCC2)[C@H](C(=O)Nc2ccnc(C(N)=O)c2)O[C@@]1(C)C(F)(F)F]

4-((2R,3S,4S,5R)-3-(3,4-difluoro-2-(2-morpholinoethoxy)-
phenyl)-4,5-dimethyl-5-(trifluoromethyl)tetrahydrofuran-2-
carboxamido)picolinamide

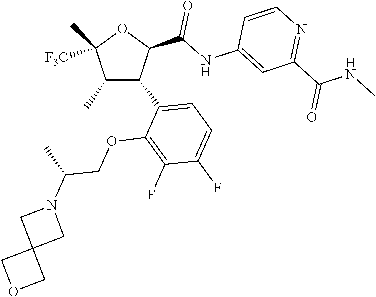 [SMILES: CNC(=O)c1cc(NC(=O)[C@@H]2O[C@@](C)(C(F)(F)F)[C@@H](C)[C@H]2c2ccc(F)c(F)c2OC[C@@H](C)N2CC3(COC3)C2)ccn1]

4-((2R,3S,4S,5R)-3-(2-((R)-2-(2-oxa-6-azaspiro[3.3]
heptan-6-yl)propoxy)-3,4-difluorophenyl)-4,5-dimethyl-
5-(trifluoro-methyl)tetrahydrofuran-2-carboxamido)-
N-methylpicolinamide

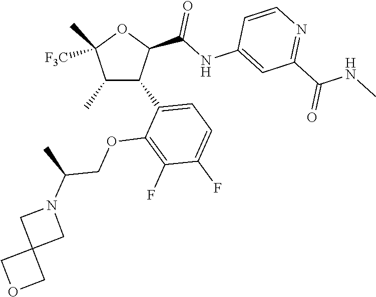 [SMILES: CNC(=O)c1cc(NC(=O)[C@@H]2O[C@@](C)(C(F)(F)F)[C@@H](C)[C@H]2c2ccc(F)c(F)c2OC[C@H](C)N2CC3(COC3)C2)ccn1]

4-((2R,3S,4S,5R)-3-(2-((S)-2-(2-oxa-6-azaspiro[3.3]heptan-
6-yl)propoxy)-3,4-difluorophenyl)-4,5-dimethyl-5-
(trifluoromethyl)tetrahydrofuran-2-carboxamido)-N-
methylpicolinamide

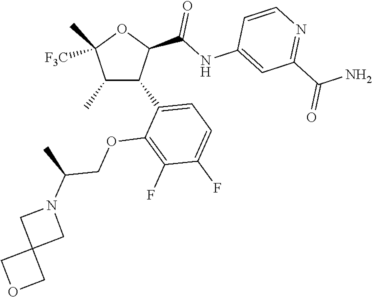 [SMILES: C[C@@H](COc1c([C@H]2[C@H](C(=O)Nc3ccnc(C(N)=O)c3)O[C@@](C)(C(F)(F)F)[C@H]2C)ccc(F)c1F)N1CC2(COC2)C1]

4-((2R,3S,4S,5R)-3-(2-((S)-2-(2-oxa-6-azaspiro[3.3]heptan-
6-yl)propoxy)-3,4-difluorophenyl)-4,5-dimethyl-5-(trifluoro-
methyl)tetrahydrofuran-2-carboxamido)picolinamide

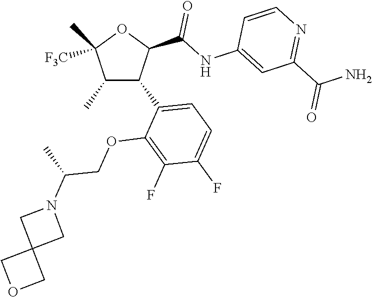 [SMILES: C[C@H](COc1c([C@H]2[C@H](C(=O)Nc3ccnc(C(N)=O)c3)O[C@@](C)(C(F)(F)F)[C@H]2C)ccc(F)c1F)N1CC2(COC2)C1]

4-((2R,3S,4S,5R)-3-(2-((R)-2-(2-oxa-6-azaspiro[3.3]heptan-
6-yl)propoxy)-3,4-difluorophenyl)-4,5-dimethyl-5-(trifluoro-
methyl)tetrahydrofuran-2-carboxamido)picolinamide

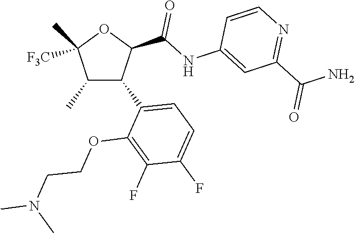 [SMILES: C[C@H]1[C@@H](c2ccc(F)c(F)c2OCCN(C)C)[C@H](C(=O)Nc2ccnc(C(N)=O)c2)O[C@@]1(C)C(F)(F)F]

4-((2R,3S,4S,5R)-3-(2-(2-(dimethylamino)ethoxy)-3,4-
difluorophenyl)-4,5-dimethyl-5-(trifluoromethyl)tetra-
hydrofuran-2-carboxamido)picolinamide

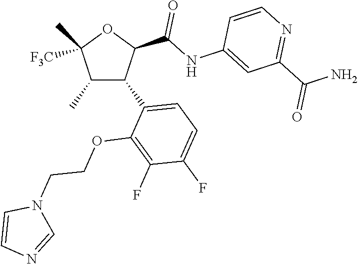 [SMILES: C[C@H]1[C@@H](c2ccc(F)c(F)c2OCCn2ccnc2)[C@H](C(=O)Nc2ccnc(C(N)=O)c2)O[C@@]1(C)C(F)(F)F]

4-((2R,3S,4S,5R)-3-(2-(2-(1H-imidazol-1-yl)ethoxy)-
3,4-difluorophenyl)-4,5-dimethyl-5-(trifluoromethyl)-
tetrahydrofuran-2-carboxamido)picolinamide

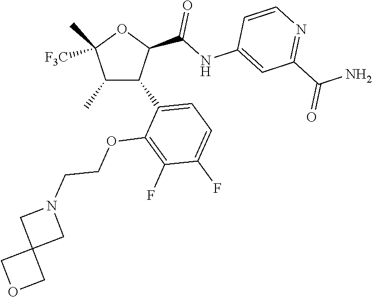 [SMILES: C[C@H]1[C@@H](c2ccc(F)c(F)c2OCCN2CC3(COC3)C2)[C@H](C(=O)Nc2ccnc(C(N)=O)c2)O[C@@]1(C)C(F)(F)F]

4-((2R,3S,4S,5R)-3-(2-(2-(2-oxa-6-azaspiro[3.3]heptan-6-
yl)ethoxy)-3,4-difluorophenyl)-4,5-dimethyl-5-(trifluoro-
methyl)tetrahydrofuran-2-carboxamido)picolinamide

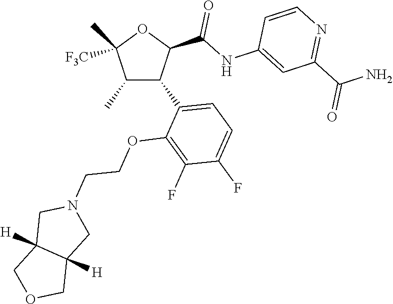 [SMILES: C[C@H]1[C@@H](c2ccc(F)c(F)c2OCCN2C[C@H]3COC[C@H]3C2)[C@H](C(=O)Nc2ccnc(C(N)=O)c2)O[C@@]1(C)C(F)(F)F]

4-((2R,3S,4S,5R)-3-(3,4-difluoro-2-(2-((3aR,6aS)-tetrahydro-
1H-furo[3,4-c]pyrrol-5(3H)-yl)ethoxy)phenyl)-4,5-dimethyl-5-
(trifluoromethyl)tetrahydrofuran-2-carboxamido)picolinamide

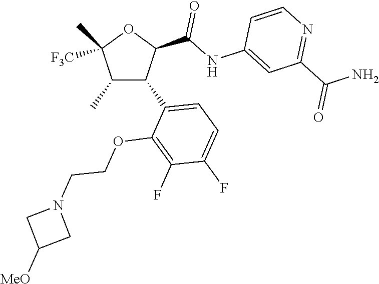 [SMILES: COC1CN(CCOc2c([C@H]3[C@H](C(=O)Nc4ccnc(C(N)=O)c4)O[C@@](C)(C(F)(F)F)[C@H]3C)ccc(F)c2F)C1]

4-((2R,3S,4S,5R)-3-(3,4-difluoro-2-(2-(3-methoxyazetidin-
1-yl)ethoxy)phenyl)-4,5-dimethyl-5-(trifluoromethyl)tetra-
hydrofuran-2-carboxamido)picolinamide

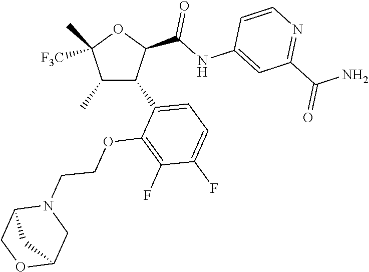 [SMILES: C[C@H]1[C@@H](c2ccc(F)c(F)c2OCCN2C[C@@H]3C[C@H]2CO3)[C@H](C(=O)Nc2ccnc(C(N)=O)c2)O[C@@]1(C)C(F)(F)F]

4-((2R,3S,4S,5R)-3-(2-(2-((1S,4S)-2-oxa-5-azabicyclo
[2.2.1]heptan-5-yl)ethoxy)-3,4-difluorophenyl)-4,5-
dimethyl-5-(trifluoromethyl)tetrahydrofuran-2-carbox-
amido)picolinamide

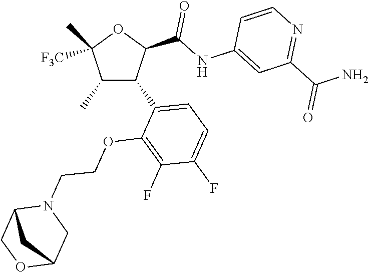 [SMILES: C[C@H]1[C@@H](c2ccc(F)c(F)c2OCCN2C[C@H]3C[C@@H]2CO3)[C@H](C(=O)Nc2ccnc(C(N)=O)c2)O[C@@]1(C)C(F)(F)F]

4-((2R,3S,4S,5R)-3-(2-(2-((1R,4R)-2-oxa-5-azabicyclo
[2.2.1]heptan-5-yl)ethoxy)-3,4-difluorophenyl)-4,5-
dimethyl-5-(trifluoromethyl)tetrahydrofuran-2-carbox-
amido)picolinamide

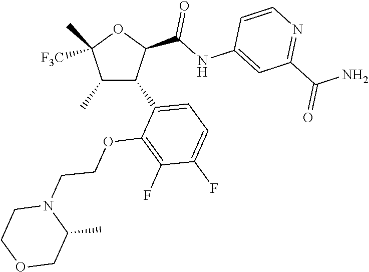 [SMILES: C[C@@H]1COCCN1CCOc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(2-((R)-3-methylmorph-
olino)ethoxy)phenyl)-4,5-dimethyl-5-(trifluoromethyl)tetra-
hydrofuran-2-carboxamido)picolinamide

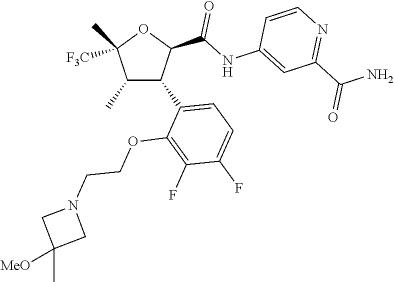 [SMILES: COC1(C)CN(CCOc2c([C@H]3[C@H](C(=O)Nc4ccnc(C(N)=O)c4)O[C@@](C)(C(F)(F)F)[C@H]3C)ccc(F)c2F)C1]

4-((2R,3S,4S,5R)-3-(3,4-difluoro-2-(2-(3-methoxy-3-methyl-
azetidin-1-yl)ethoxy)phenyl)-4,5-dimethyl-5-(trifluoromethyl)-
tetrahydrofuran-2-carboxamido)picolinamide

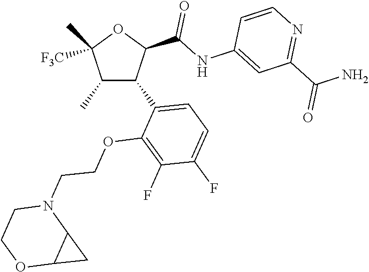 [SMILES: C[C@H]1[C@@H](c2ccc(F)c(F)c2OCCN2CCOC3CC32)[C@H](C(=O)Nc2ccnc(C(N)=O)c2)O[C@@]1(C)C(F)(F)F]

4-((2R,3S,4S,5R)-3-(2-(2-(2-oxa-5-azabicyclo[4.1.0]heptan-
5-yl)ethoxy)-3,4-difluorophenyl)-4,5-dimethyl-5-(trifluoro-
methyl)tetrahydrofuran-2-carboxamido)picolinamide

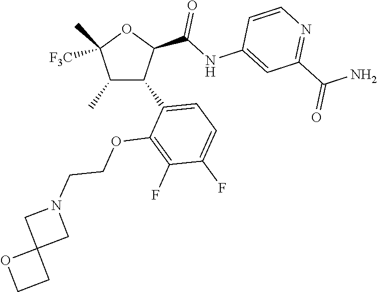 [SMILES: C[C@H]1[C@@H](c2ccc(F)c(F)c2OCCN2CC3(CCO3)C2)[C@H](C(=O)Nc2ccnc(C(N)=O)c2)O[C@@]1(C)C(F)(F)F]

4-((2R,3S,4S,5R)-3-(2-(2-(1-oxa-6-azaspiro[3.3]heptan-6-
yl)ethoxy)-3,4-difluorophenyl)-4,5-dimethyl-5-(trifluoro-
methyl)tetrahydrofuran-2-carboxamido)picolinamide

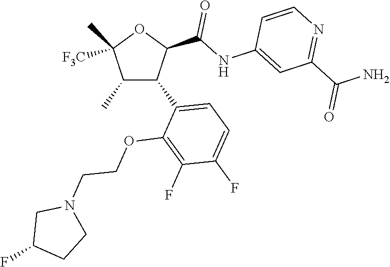 [SMILES: C[C@H]1[C@@H](c2ccc(F)c(F)c2OCCN2CC[C@H](F)C2)[C@H](C(=O)Nc2ccnc(C(N)=O)c2)O[C@@]1(C)C(F)(F)F]

4-((2R,3S,4S,5R)-3-(3,4-difluoro-2-(2-((S)-3-fluoropyrrolidin-
1-yl)ethoxy)phenyl)-4,5-dimethyl-5-(trifluoromethyl)tetra-
hydrofuran-2-carboxamido)picolinamide

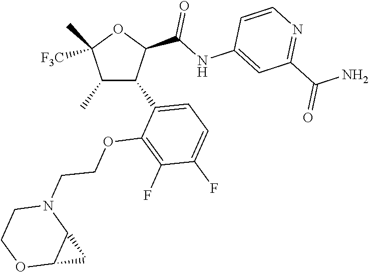

4-((2R,3S,4S,5R)-3-(2-(2-((1S,6R)-2-oxa-5-azabicyclo
[4.1.0]heptan-5-yl)ethoxy)-3,4-difluorophenyl)-4,5-
dimethyl-5-(trifluoromethyl)tetrahydrofuran-2-carbox-
amido)picolinamide

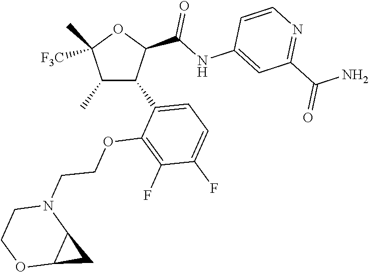 [SMILES: C[C@H]1[C@@H](c2ccc(F)c(F)c2OCCN2CCO[C@@H]3C[C@@H]32)[C@H](C(=O)Nc2ccnc(C(N)=O)c2)O[C@@]1(C)C(F)(F)F]

4-((2R,3S,4S,5R)-3-(2-(2-((1R,6S)-2-oxa-5-azabicyclo
[4.1.0]heptan-5-yl)ethoxy)-3,4-difluorophenyl)-4,5-
dimethyl-5-(trifluoromethyl)tetrahydrofuran-2-carbox-
amido)picolinamide

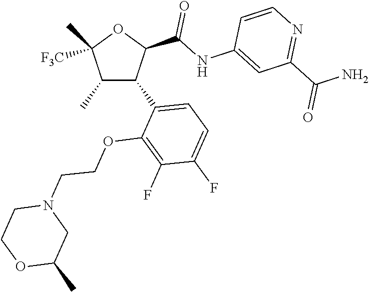 [SMILES: C[C@@H]1CN(CCOc2c([C@H]3[C@H](C(=O)Nc4ccnc(C(N)=O)c4)O[C@@](C)(C(F)(F)F)[C@H]3C)ccc(F)c2F)CCO1]

4-((2R,3S,4S,5R)-3-(3,4-difluoro-2-(2-((R)-2-methyl-
morpholino)ethoxy)phenyl)-4,5-dimethyl-5-(trifluoro-
methyl)tetrahydrofuran-2-carboxamido)picolinamide

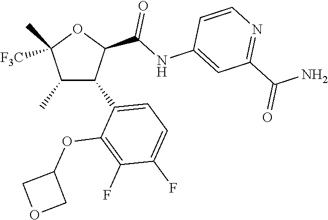 [SMILES: C[C@H]1[C@@H](c2ccc(F)c(F)c2OC2COC2)[C@H](C(=O)Nc2ccnc(C(N)=O)c2)O[C@@]1(C)C(F)(F)F]

4-((2R,3S,4S,5R)-3-(3,4-difluoro-2-(oxetan-3-yl-
oxy)phenyl)-4,5-dimethyl-5-(trifluoromethyl)tetra-
hydrofuran-2-carboxamido)picolinamide

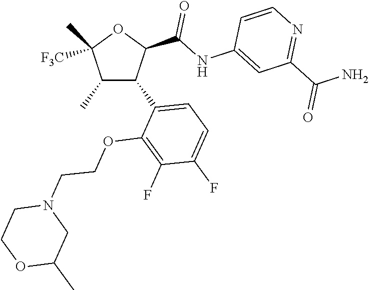 [SMILES: CC1CN(CCOc2c([C@H]3[C@H](C(=O)Nc4ccnc(C(N)=O)c4)O[C@@](C)(C(F)(F)F)[C@H]3C)ccc(F)c2F)CCO1]

4-((2R,3S,4S,5R)-3-(3,4-difluoro-2-(2-(2-methylmorpho-
lino)ethoxy)phenyl)-4,5-dimethyl-5-(trifluoromethyl)tetra-
hydrofuran-2-carboxamido)picolinamide

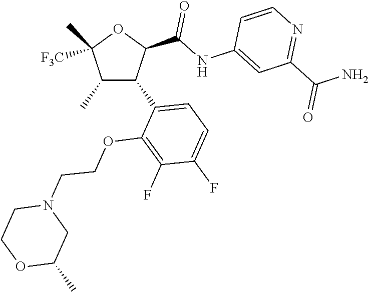 [SMILES: C[C@H]1CN(CCOc2c([C@H]3[C@H](C(=O)Nc4ccnc(C(N)=O)c4)O[C@@](C)(C(F)(F)F)[C@H]3C)ccc(F)c2F)CCO1]

4-((2R,3S,4S,5R)-3-(3,4-difluoro-2-(2-((S)-2-methyl-
morpholino)ethoxy)phenyl)-4,5-dimethyl-5-(trifluoro-
methyl)tetrahydrofuran-2-carboxamido)picolinamide

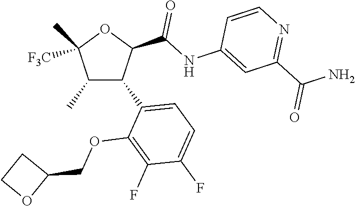 [SMILES: C[C@H]1[C@@H](c2ccc(F)c(F)c2OC[C@@H]2CCO2)[C@H](C(=O)Nc2ccnc(C(N)=O)c2)O[C@@]1(C)C(F)(F)F]

4-((2R,3S,4S,5R)-3-(3,4-difluoro-2-(((S)-oxetan-2-yl)
methoxy)phenyl)-4,5-dimethyl-5-(trifluoromethyl)tetra-
hydrofuran-2-carboxamido)picolinamide

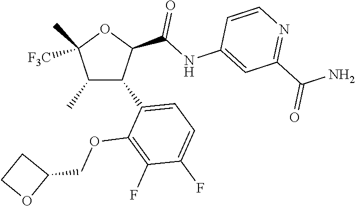 [SMILES: C[C@H]1[C@@H](c2ccc(F)c(F)c2OC[C@H]2CCO2)[C@H](C(=O)Nc2ccnc(C(N)=O)c2)O[C@@]1(C)C(F)(F)F]

4-((2R,3S,4S,5R)-3-(3,4-difluoro-2-(((R)-oxetan-2-yl)-
methoxy)phenyl)-4,5-dimethyl-5-(trifluoromethyl)tetra-
hydrofuran-2-carboxamido)picolinamide

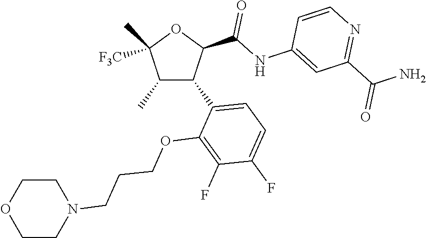 [SMILES: C[C@H]1[C@@H](c2ccc(F)c(F)c2OCCCN2CCOCC2)[C@H](C(=O)Nc2ccnc(C(N)=O)c2)O[C@@]1(C)C(F)(F)F]

4-((2R,3S,4S,5R)-3-(3,4-difluoro-2-(3-morpholinopropoxy)phenyl)-
4,5-dimethyl-5-(trifluoromethyl)tetrahydrofuran-2-carboxamido)-
picolinamide

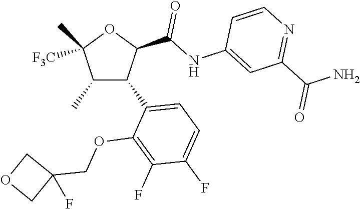 [SMILES: C[C@H]1[C@@H](c2ccc(F)c(F)c2OCC2(F)COC2)[C@H](C(=O)Nc2ccnc(C(N)=O)c2)O[C@@]1(C)C(F)(F)F]

4-((2R,3S,4S,5R)-3-(3,4-difluoro-2-((3-fluorooxetan-3-
yl)methoxy)phenyl)-4,5-dimethyl-5-(trifluoromethyl)-
tetrahydrofuran-2-carboxamido)picolinamide

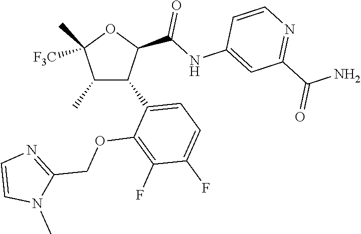 [SMILES: C[C@H]1[C@@H](c2ccc(F)c(F)c2OCc2nccn2C)[C@H](C(=O)Nc2ccnc(C(N)=O)c2)O[C@@]1(C)C(F)(F)F]

4-((2R,3S,4S,5R)-3-(3,4-difluoro-2-((1-methyl-1H-imid-
azol-2-yl)methoxy)phenyl)-4,5-dimethyl-5-(trifluorometh-
yl)tetrahydrofuran-2-carboxamido)picolinamide

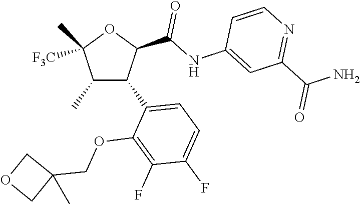 [SMILES: C[C@H]1[C@@H](c2ccc(F)c(F)c2OCC2(C)COC2)[C@H](C(=O)Nc2ccnc(C(N)=O)c2)O[C@@]1(C)C(F)(F)F]

4-((2R,3S,4S,5R)-3-(3,4-difluoro-2-((3-methyloxetan-3-
yl)methoxy)phenyl)-4,5-dimethyl-5-(trifluoromethyl)-
tetrahydrofuran-2-carboxamido)picolinamide

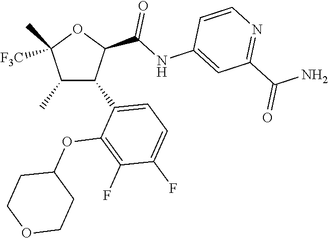 [SMILES: C[C@H]1[C@@H](c2ccc(F)c(F)c2OC2CCOCC2)[C@H](C(=O)Nc2ccnc(C(N)=O)c2)O[C@@]1(C)C(F)(F)F]

4-((2R,3S,4S,5R)-3-(3,4-difluoro-2-((tetrahydro-2H-
pyran-4-yl)oxy)phenyl)-4,5-dimethyl-5-(trifluoro-
methyl)tetrahydrofuran-2-carboxamido)picolinamide

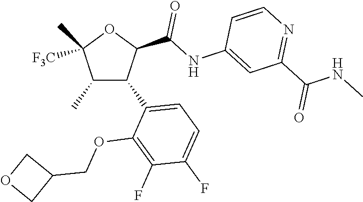 [SMILES: CNC(=O)c1cc(NC(=O)[C@@H]2O[C@@](C)(C(F)(F)F)[C@@H](C)[C@H]2c2ccc(F)c(F)c2OCC2COC2)ccn1]

4-((2R,3S,4S,5R)-3-(3,4-difluoro-2-(oxetan-3-ylmethoxy)-
phenyl)-4,5-dimethyl-5-(trifluoromethyl)tetrahydrofuran-
2-carboxamido)-N-methylpicolinamide

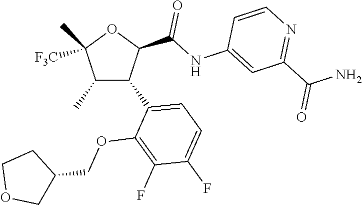 [SMILES: C[C@H]1[C@@H](c2ccc(F)c(F)c2OC[C@H]2CCOC2)[C@H](C(=O)Nc2ccnc(C(N)=O)c2)O[C@@]1(C)C(F)(F)F]

4-((2R,3S,4S,5R)-3-(3,4-difluoro-2-(((S)-tetrahydrofuran-
3-yl)methoxy)phenyl)-4,5-dimethyl-5-(trifluoromethyl)-
tetrahydrofuran-2-carboxamido)picolinamide

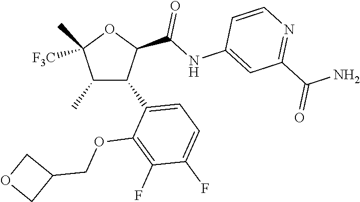 [SMILES: C[C@H]1[C@@H](c2ccc(F)c(F)c2OCC2COC2)[C@H](C(=O)Nc2ccnc(C(N)=O)c2)O[C@@]1(C)C(F)(F)F]

4-((2R,3S,4S,5R)-3-(3,4-difluoro-2-(oxetan-3-ylmethoxy)-
phenyl)-4,5-dimethyl-5-(trifluoromethyl)tetrahydrofuran-
2-carboxamido)picolinamide

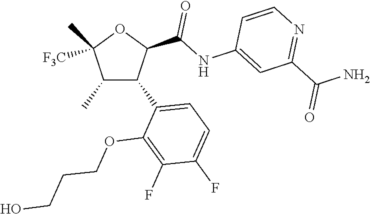 [SMILES: C[C@H]1[C@@H](c2ccc(F)c(F)c2OCCCO)[C@H](C(=O)Nc2ccnc(C(N)=O)c2)O[C@@]1(C)C(F)(F)F]

4-((2R,3S,4S,5R)-3-(3,4-difluoro-2-(3-hydroxypropoxy)-
phenyl)-4,5-dimethyl-5-(trifluoromethyl)tetrahydrofuran-
2-carboxamido)picolinamide

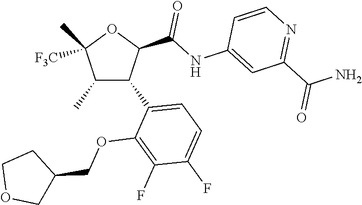 [SMILES: C[C@H]1[C@@H](c2ccc(F)c(F)c2OC[C@@H]2CCOC2)[C@H](C(=O)Nc2ccnc(C(N)=O)c2)O[C@@]1(C)C(F)(F)F]

4-((2R,3S,4S,5R)-3-(3,4-difluoro-2-(((R)-tetrahydrofuran-
3-yl)methoxy)phenyl)-4,5-dimethyl-5-(trifluoromethyl)-
tetrahydrofuran-2-carboxamido)picolinamide

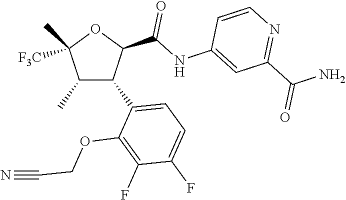 [SMILES: C[C@H]1[C@@H](c2ccc(F)c(F)c2OCC#N)[C@H](C(=O)Nc2ccnc(C(N)=O)c2)O[C@@]1(C)C(F)(F)F]

4-((2R,3S,4S,5R)-3-(2-(cyanomethoxy)-3,4-difluoro-
phenyl)-4,5-dimethyl-5-(trifluoromethyl)tetrahydro-
furan-2-carboxamido)picolinamide

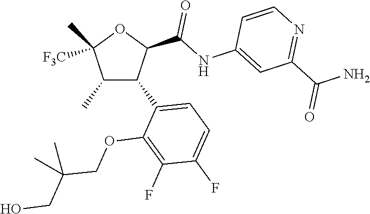 [SMILES: C[C@H]1[C@@H](c2ccc(F)c(F)c2OCC(C)(C)CO)[C@H](C(=O)Nc2ccnc(C(N)=O)c2)O[C@@]1(C)C(F)(F)F]

4-((2R,3S,4S,5R)-3-(3,4-difluoro-2-(3-hydroxy-2,2-dimeth-
ylpropoxy)phenyl)-4,5-dimethyl-5-(trifluoromethyl)tetra-
hydrofuran-2-carboxamido)picolinamide

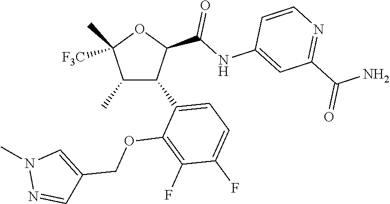 [SMILES: C[C@H]1[C@@H](c2ccc(F)c(F)c2OCc2cnn(C)c2)[C@H](C(=O)Nc2ccnc(C(N)=O)c2)O[C@@]1(C)C(F)(F)F]

4-((2R,3S,4S,5R)-3-(3,4-difluoro-2-((1-methyl-1H-pyrazol-
4-yl)methoxy)phenyl)-4,5-dimethyl-5-(trifluoromethyl)tetra-
hydrofuran-2-carboxamido)picolinamide

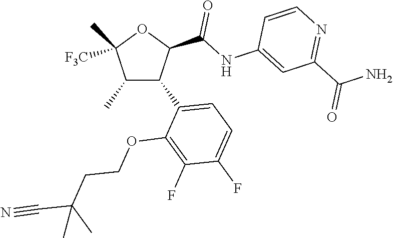 [SMILES: C[C@H]1[C@@H](c2ccc(F)c(F)c2OCCC(C)(C)C#N)[C@H](C(=O)Nc2ccnc(C(N)=O)c2)O[C@@]1(C)C(F)(F)F]

4-((2R,3S,4S,5R)-3-(2-(3-cyano-3-methylbutoxy)-3,4-difluoro-
phenyl)-4,5-dimethyl-5-(trifluoromethyl)tetrahydrofuran-2-
carboxamido)picolinamide

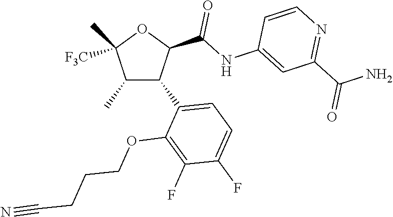 [SMILES: C[C@H]1[C@@H](c2ccc(F)c(F)c2OCCCC#N)[C@H](C(=O)Nc2ccnc(C(N)=O)c2)O[C@@]1(C)C(F)(F)F]

4-((2R,3S,4S,5R)-3-(2-(3-cyanopropoxy)-3,4-difluorophen-
yl)-4,5-dimethyl-5-(trifluoromethyl)tetrahydrofuran-2-carbox-
amido)picolinamide

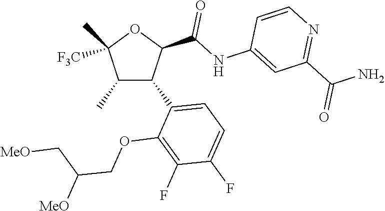 [SMILES: COCC(COc1c([C@H]2[C@H](C(=O)Nc3ccnc(C(N)=O)c3)O[C@@](C)(C(F)(F)F)[C@H]2C)ccc(F)c1F)OC]

4-((2R,3S,4S,5R)-3-(2-(2,3-dimethoxypropoxy)-3,4-difluoro-
phenyl)-4,5-dimethyl-5-(trifluoromethyl)tetrahydrofuran-2-
carboxamido)picolinamide

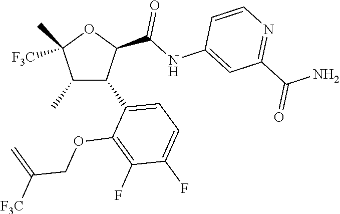 [SMILES: C=C(COc1c([C@H]2[C@H](C(=O)Nc3ccnc(C(N)=O)c3)O[C@@](C)(C(F)(F)F)[C@H]2C)ccc(F)c1F)C(F)(F)F]

4-((2R,3S,4S,5R)-3-(3,4-difluoro-2-((2-(trifluorometh-
yl)allyl)oxy)phenyl)-4,5-dimethyl-5-(trifluoromethyl)-
tetrahydrofuran-2-carboxamido)picolinamide

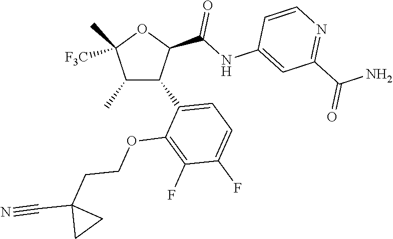 [SMILES: C[C@H]1[C@@H](c2ccc(F)c(F)c2OCCC2(C#N)CC2)[C@H](C(=O)Nc2ccnc(C(N)=O)c2)O[C@@]1(C)C(F)(F)F]

4-((2R,3S,4S,5R)-3-(2-(2-(1-cyanocyclopropyl)ethoxy)-3,4-
difluorophenyl)-4,5-dimethyl-5-(trifluoromethyl)tetrahydro-
furan-2-carboxamido)picolinamide

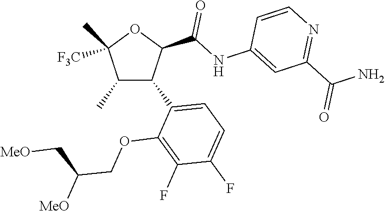 [SMILES: COC[C@@H](COc1c([C@H]2[C@H](C(=O)Nc3ccnc(C(N)=O)c3)O[C@@](C)(C(F)(F)F)[C@H]2C)ccc(F)c1F)OC]

4-((2R,3S,4S,5R)-3-(2-((S)-2,3-dimethoxypropoxy)-3,4-
difluorophenyl)-4,5-dimethyl-5-(trifluoromethyl)tetrahydro-
furan-2-carboxamido)picolinamide

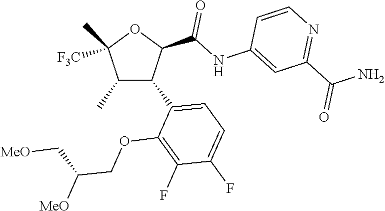 [SMILES: COC[C@H](COc1c([C@H]2[C@H](C(=O)Nc3ccnc(C(N)=O)c3)O[C@@](C)(C(F)(F)F)[C@H]2C)ccc(F)c1F)OC]

4-((2R,3S,4S,5R)-3-(2-((R)-2,3-dimethoxypropoxy)-3,4-
difluorophenyl)-4,5-dimethyl-5-(trifluoromethyl)tetrahydro-
furan-2-carboxamido)picolinamide

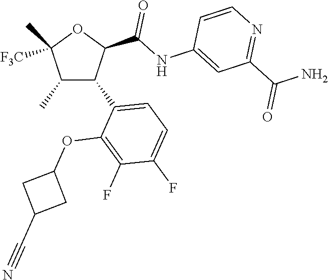 [SMILES: C[C@H]1[C@@H](c2ccc(F)c(F)c2OC2CC(C#N)C2)[C@H](C(=O)Nc2ccnc(C(N)=O)c2)O[C@@]1(C)C(F)(F)F]

4-((2R,3S,4S,5R)-3-(2-(3-cyanocyclobutoxy)-3,4-
difluorophenyl)-4,5-dimethyl-5-(trifluoromethyl)-
tetrahydrofuran-2-carboxamido)picolinamide

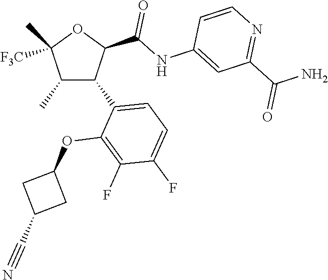 [SMILES: C[C@H]1[C@@H](c2ccc(F)c(F)c2O[C@H]2C[C@H](C#N)C2)[C@H](C(=O)Nc2ccnc(C(N)=O)c2)O[C@@]1(C)C(F)(F)F]

4-((2R,3S,4S,5R)-3-(2-((1r,3S)-3-cyanocyclobutoxy)-
3,4-difluorophenyl)-4,5-dimethyl-5-(trifluoromethyl)-
tetrahydrofuran-2-carboxamido)picolinamide

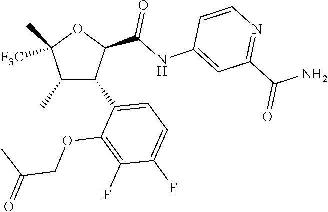 [SMILES: CC(=O)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(2-oxopropoxy)-
phenyl)-4,5-dimethyl-5-(trifluoromethyl)tetrahydro-
furan-2-carboxamido)picolinamide

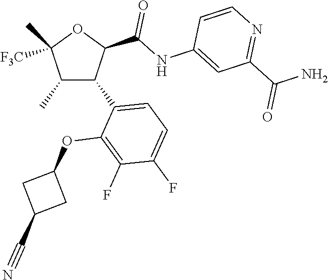 [SMILES: C[C@H]1[C@@H](c2ccc(F)c(F)c2O[C@H]2C[C@@H](C#N)C2)[C@H](C(=O)Nc2ccnc(C(N)=O)c2)O[C@@]1(C)C(F)(F)F]

4-((2R,3S,4S,5R)-3-(2-((1s,3R)-3-cyanocyclobutoxy)-
3,4-difluorophenyl)-4,5-dimethyl-5-(trifluoromethyl)-
tetrahydrofuran-2-carboxamido)picolinamide

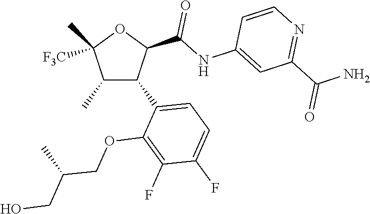 [SMILES: C[C@@H](CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((S)-3-hydroxy-2-meth-
ylpropoxy)phenyl)-4,5-dimethyl-5-(trifluoromethyl)tetra-
hydrofuran-2-carboxamido)picolinamide

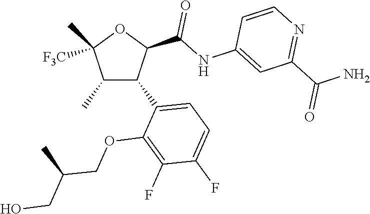 [SMILES: C[C@H](CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((R)-3-hydroxy-2-meth-
ylpropoxy)phenyl)-4,5-dimethyl-5-(trifluoromethyl)tetra-
hydrofuran-2-carboxamido)picolinamide

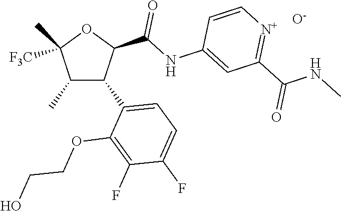

4-((2R,3S,4S,5R)-3-(3,4-difluoro-2-(2-hydroxyethoxy)-
phenyl)-4,5-dimethyl-5-(trifluoromethyl)tetrahydro-
furan-2-carboxamido)-2-(methylcarbamoyl)pyridine
1-oxide

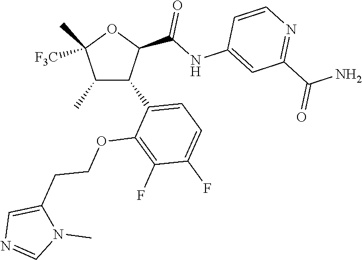 [SMILES: C[C@H]1[C@@H](c2ccc(F)c(F)c2OCCc2cncn2C)[C@H](C(=O)Nc2ccnc(C(N)=O)c2)O[C@@]1(C)C(F)(F)F]

4-((2R,3S,4S,5R)-3-(3,4-difluoro-2-(2-(1-methyl-1H-imid-
azol-5-yl)ethoxy)phenyl)-4,5-dimethyl-5-(trifluoromethyl)-
tetrahydrofuran-2-carboxamido)picolinamide

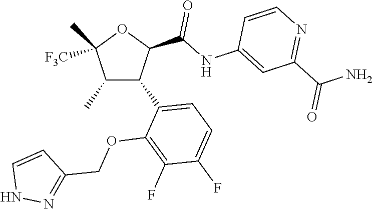 [SMILES: C[C@H]1[C@@H](c2ccc(F)c(F)c2OCc2cc[nH]n2)[C@H](C(=O)Nc2ccnc(C(N)=O)c2)O[C@@]1(C)C(F)(F)F]

4-((2R,3S,4S,5R)-3-(2-((1H-pyrazol-3-yl)methoxy)-3,4-
difluorophenyl)-4,5-dimethyl-5-(trifluoromethyl)tetra-
hydrofuran-2-carboxamido)picolinamide

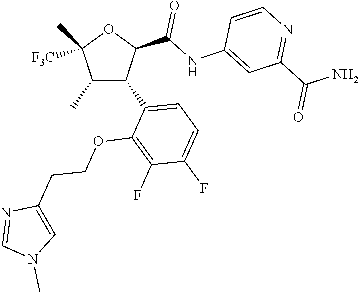 [SMILES: C[C@H]1[C@@H](c2ccc(F)c(F)c2OCCc2cn(C)cn2)[C@H](C(=O)Nc2ccnc(C(N)=O)c2)O[C@@]1(C)C(F)(F)F]

4-((2R,3S,4S,5R)-3-(3,4-difluoro-2-(2-(1-methyl-1H-
imidazol-4-yl)ethoxy)phenyl)-4,5-dimethyl-5-(trifluoro-
methyl)tetrahydrofuran-2-carboxamido)picolinamide

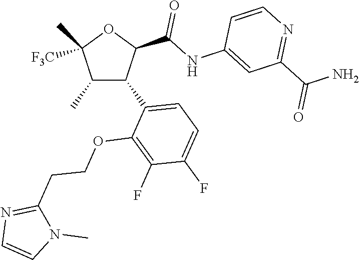 [SMILES: C[C@H]1[C@@H](c2ccc(F)c(F)c2OCCc2nccn2C)[C@H](C(=O)Nc2ccnc(C(N)=O)c2)O[C@@]1(C)C(F)(F)F]

4-((2R,3S,4S,5R)-3-(3,4-difluoro-2-(2-(1-methyl-1H-
imidazol-2-yl)ethoxy)phenyl)-4,5-dimethyl-5-(trifluoro-
methyl)tetrahydrofuran-2-carboxamido)picolinamide

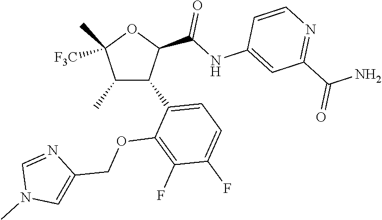 [SMILES: C[C@H]1[C@@H](c2ccc(F)c(F)c2OCc2cn(C)cn2)[C@H](C(=O)Nc2ccnc(C(N)=O)c2)O[C@@]1(C)C(F)(F)F]

4-((2R,3S,4S,5R)-3-(3,4-difluoro-2-((1-methyl-1H-imidazol-
4-yl)methoxy)phenyl)-4,5-dimethyl-5-(trifluoromethyl)tetra-
hydrofuran-2-carboxamido)picolinamide

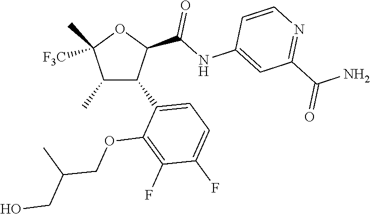 [SMILES: CC(CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(3-hydroxy-2-methyl-
propoxy)phenyl)-4,5-dimethyl-5-(trifluoromethyl)tetra-
hydrofuran-2-carboxamido)picolinamide

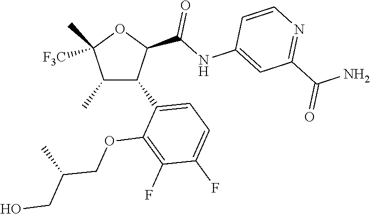 [SMILES: C[C@@H](CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((S)-3-hydroxy-2-meth-
ylpropoxy)phenyl)-4,5-dimethyl-5-(trifluoromethyl)tetra-
hydrofuran-2-carboxamido)picolinamide

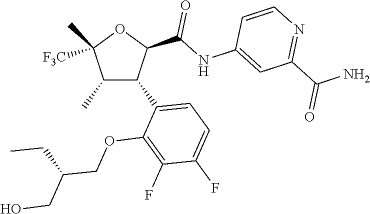 [SMILES: CC[C@@H](CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((S)-2-(hydroxymethyl)-
butoxy)phenyl)-4,5-dimethyl-5-(trifluoromethyl)tetrahydro-
furan-2-carboxamido)picolinamide

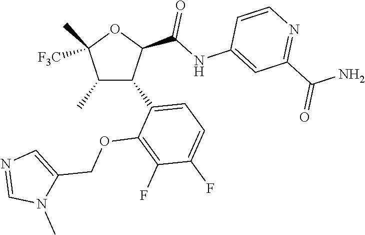 [SMILES: C[C@H]1[C@@H](c2ccc(F)c(F)c2OCc2cncn2C)[C@H](C(=O)Nc2ccnc(C(N)=O)c2)O[C@@]1(C)C(F)(F)F]

4-((2R,3S,4S,5R)-3-(3,4-difluoro-2-((1-methyl-1H-imidazol-
5-yl)methoxy)phenyl)-4,5-dimethyl-5-(trifluoromethyl)tetra-
hydrofuran-2-carboxamido)picolinamide

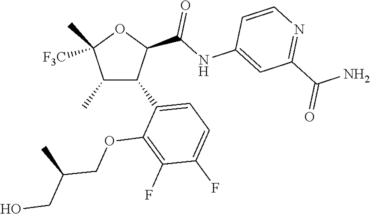 [SMILES: C[C@H](CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((R)-3-hydroxy-2-meth-
ylpropoxy)phenyl)-4,5-dimethyl-5-(trifluoromethyl)tetra-
hydrofuran-2-carboxamido)picolinamide

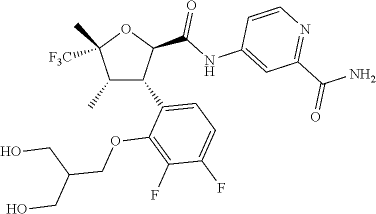 [SMILES: C[C@H]1[C@@H](c2ccc(F)c(F)c2OCC(CO)CO)[C@H](C(=O)Nc2ccnc(C(N)=O)c2)O[C@@]1(C)C(F)(F)F]

4-((2R,3S,4S,5R)-3-(3,4-difluoro-2-(3-hydroxy-2-(hydroxy-
methyl)propoxy)phenyl)-4,5-dimethyl-5-(trifluoromethyl)-
tetrahydrofuran-2-carboxamido)picolinamide

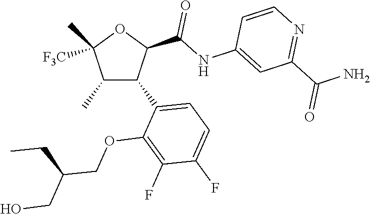 [SMILES: CC[C@H](CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((R)-2-(hydroxymethyl)-
butoxy)phenyl)-4,5-dimethyl-5-(trifluoromethyl)tetrahydro-
furan-2-carboxamido)picolinamide

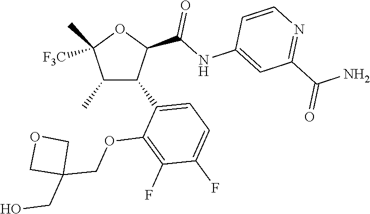 [SMILES: C[C@H]1[C@@H](c2ccc(F)c(F)c2OCC2(CO)COC2)[C@H](C(=O)Nc2ccnc(C(N)=O)c2)O[C@@]1(C)C(F)(F)F]

4-((2R,3S,4S,5R)-3-(3,4-difluoro-2-((3-(hydroxymethyl)-
oxetan-3-yl)methoxy)phenyl)-4,5-dimethyl-5-(trifluoro-
methyl)tetrahydrofuran-2-carboxamido)picolinamide

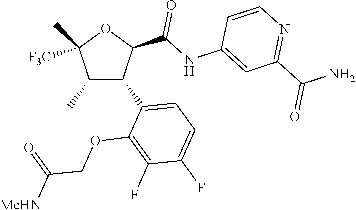 [SMILES: CNC(=O)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(2-(methylamino)-2-
oxoethoxy)phenyl)-4,5-dimethyl-5-(trifluoromethyl)tetra-
hydrofuran-2-carboxamido)picolinamide

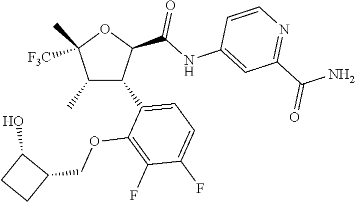 [SMILES: C[C@H]1[C@@H](c2ccc(F)c(F)c2OC[C@@H]2CC[C@@H]2O)[C@H](C(=O)Nc2ccnc(C(N)=O)c2)O[C@@]1(C)C(F)(F)F]

4-((2R,3S,4S,5R)-3-(3,4-difluoro-2-(((1S,2S)-2-hydroxy-
cyclobutyl)methoxy)phenyl)-4,5-dimethyl-5-(trifluoro-
methyl)tetrahydrofuran-2-carboxamido)picolinamide

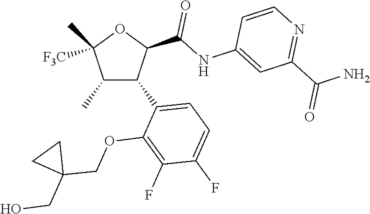 [SMILES: C[C@H]1[C@@H](c2ccc(F)c(F)c2OCC2(CO)CC2)[C@H](C(=O)Nc2ccnc(C(N)=O)c2)O[C@@]1(C)C(F)(F)F]

4-((2R,3S,4S,5R)-3-(3,4-difluoro-2-((1-(hydroxymethyl)-
cyclopropyl)methoxy)phenyl)-4,5-dimethyl-5-(trifluoro-
methyl)tetrahydrofuran-2-carboxamido)picolinamide

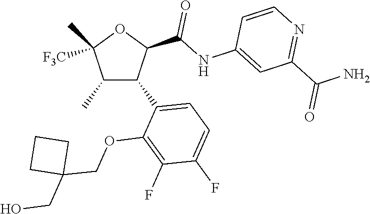 [SMILES: C[C@H]1[C@@H](c2ccc(F)c(F)c2OCC2(CO)CCC2)[C@H](C(=O)Nc2ccnc(C(N)=O)c2)O[C@@]1(C)C(F)(F)F]

4-((2R,3S,4S,5R)-3-(3,4-difluoro-2-((1-(hydroxymethyl)-
cyclobutyl)methoxy)phenyl)-4,5-dimethyl-5-(trifluoro-
methyl)tetrahydrofuran-2-carboxamido)picolinamide

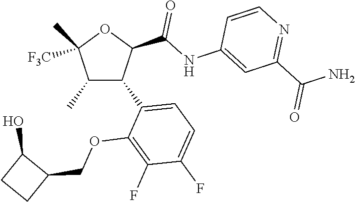 [SMILES: C[C@H]1[C@@H](c2ccc(F)c(F)c2OC[C@H]2CC[C@H]2O)[C@H](C(=O)Nc2ccnc(C(N)=O)c2)O[C@@]1(C)C(F)(F)F]

4-((2R,3S,4S,5R)-3-(3,4-difluoro-2-(((1R,2R)-2-hydroxy-
cyclobutyl)methoxy)phenyl)-4,5-dimethyl-5-(trifluoro-
methyl)tetrahydrofuran-2-carboxamido)picolinamide

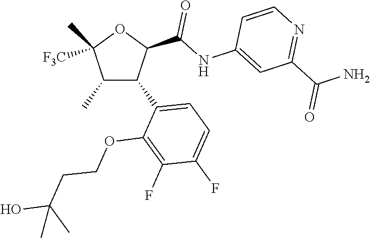 [SMILES: C[C@H]1[C@@H](c2ccc(F)c(F)c2OCCC(C)(C)O)[C@H](C(=O)Nc2ccnc(C(N)=O)c2)O[C@@]1(C)C(F)(F)F]

4-((2R,3S,4S,5R)-3-(3,4-difluoro-2-(3-hydroxy-3-methyl-
butoxy)phenyl)-4,5-dimethyl-5-(trifluoromethyl)tetrahydro-
furan-2-carboxamido)picolinamide

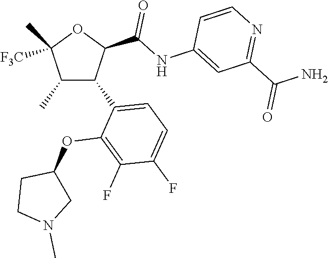 [SMILES: C[C@H]1[C@@H](c2ccc(F)c(F)c2O[C@@H]2CCN(C)C2)[C@H](C(=O)Nc2ccnc(C(N)=O)c2)O[C@@]1(C)C(F)(F)F]

4-((2R,3S,4S,5R)-3-(3,4-difluoro-2-(((R)-1-methyl-
pyrrolidin-3-yl)oxy)phenyl)-4,5-dimethyl-5-(tri-
fluoromethyl)tetrahydrofuran-2-carboxamido)-
picolinamide

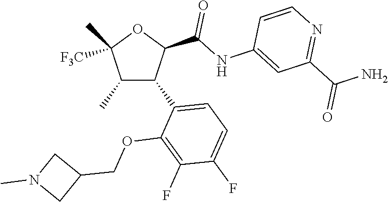 [SMILES: C[C@H]1[C@@H](c2ccc(F)c(F)c2OCC2CN(C)C2)[C@H](C(=O)Nc2ccnc(C(N)=O)c2)O[C@@]1(C)C(F)(F)F]

4-((2R,3S,4S,5R)-3-(3,4-difluoro-2-((1-methylazetidin-3-yl)-
methoxy)phenyl)-4,5-dimethyl-5-(trifluoromethyl)tetrahydro-
furan-2-carboxamido)picolinamide

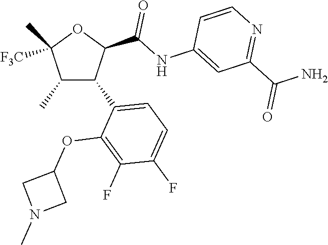 [SMILES: C[C@H]1[C@@H](c2ccc(F)c(F)c2OC2CN(C)C2)[C@H](C(=O)Nc2ccnc(C(N)=O)c2)O[C@@]1(C)C(F)(F)F]

4-((2R,3S,4S,5R)-3-(3,4-difluoro-2-((1-methyl
azetidin-3-yl)oxy)phenyl)-4,5-dimethyl-5-(trifluoro-
methyl)tetrahydrofuran-2-carboxamido)-
picolinamide

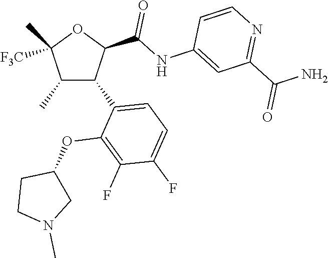 [SMILES: C[C@H]1[C@@H](c2ccc(F)c(F)c2O[C@H]2CCN(C)C2)[C@H](C(=O)Nc2ccnc(C(N)=O)c2)O[C@@]1(C)C(F)(F)F]

4-((2R,3S,4S,5R)-3-(3,4-difluoro-2-(((S)-1-methyl-
pyrrolidin-3-yl)oxy)phenyl)-4,5-dimethyl-5-
(trifluoromethyl)tetrahydrofuran-2-carboxamido)-
picolinamide

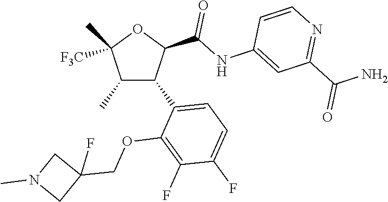 [SMILES: C[C@H]1[C@@H](c2ccc(F)c(F)c2OCC2(F)CN(C)C2)[C@H](C(=O)Nc2ccnc(C(N)=O)c2)O[C@@]1(C)C(F)(F)F]

4-((2R,3S,4S,5R)-3-(3,4-difluoro-2-((3-fluoro-1-methyl-
azetidin-3-yl)methoxy)phenyl)-4,5-dimethyl-5-(trifluoro-
methyl)tetrahydrofuran-2-carboxamido)picolinamide

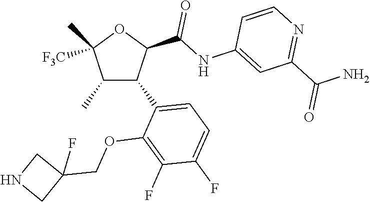 [SMILES: C[C@H]1[C@@H](c2ccc(F)c(F)c2OCC2(F)CNC2)[C@H](C(=O)Nc2ccnc(C(N)=O)c2)O[C@@]1(C)C(F)(F)F]

4-((2R,3S,4S,5R)-3-(3,4-difluoro-2-((3-fluoroazetidin-3-yl)-
methoxy)phenyl)-4,5-dimethyl-5-(trifluoromethyl)tetrahydro-
furan-2-carboxamido)picolinamide

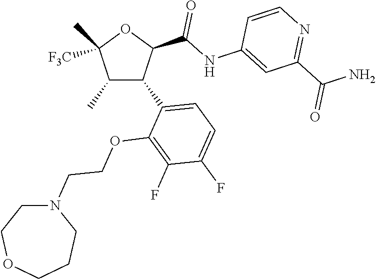 [SMILES: C[C@H]1[C@@H](c2ccc(F)c(F)c2OCCN2CCCOCC2)[C@H](C(=O)Nc2ccnc(C(N)=O)c2)O[C@@]1(C)C(F)(F)F]

4-((2R,3S,4S,5R)-3-(2-(2-(1,4-oxazepan-4-yl)ethoxy)-3,4-
difluorophenyl)-4,5-dimethyl-5-(trifluoromethyl)tetrahydro-
furan-2-carboxamido)picolinamide

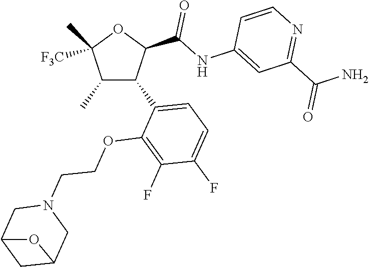 [SMILES: C[C@H]1[C@@H](c2ccc(F)c(F)c2OCCN2CC3CC(C2)O3)[C@H](C(=O)Nc2ccnc(C(N)=O)c2)O[C@@]1(C)C(F)(F)F]

4-((2R,3S,4S,5R)-3-(2-(2-(6-oxa-3-azabicyclo[3.1.1]heptan-
3-yl)ethoxy)-3,4-difluorophenyl)-4,5-dimethyl-5-(trifluoro-
methyl)tetrahydrofuran-2-carboxamido)picolinamide

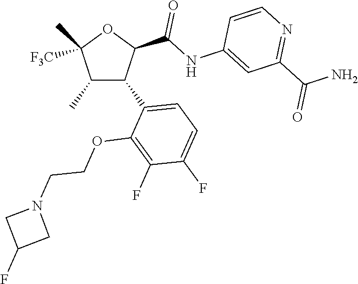 [SMILES: C[C@H]1[C@@H](c2ccc(F)c(F)c2OCCN2CC(F)C2)[C@H](C(=O)Nc2ccnc(C(N)=O)c2)O[C@@]1(C)C(F)(F)F]

4-((2R,3S,4S,5R)-3-(3,4-difluoro-2-(2-(3-fluoroazetidin-
1-yl)ethoxy)phenyl)-4,5-dimethyl-5-(trifluoromethyl)-
tetrahydrofuran-2-carboxamido)picolinamide

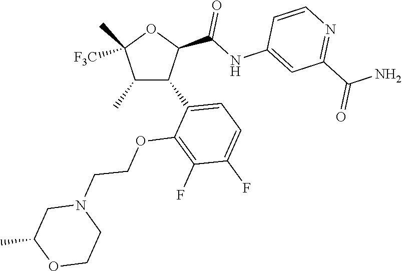 [SMILES: C[C@@H]1CN(CCOc2c([C@H]3[C@H](C(=O)Nc4ccnc(C(N)=O)c4)O[C@@](C)(C(F)(F)F)[C@H]3C)ccc(F)c2F)CCO1]

4-((2R,3S,4S,5R)-3-(3,4-difluoro-2-(2-((R)-2-methylmorpholino)-
ethoxy)phenyl)-4,5-dimethyl-5-(trifluoromethyl)tetrahydrofuran-
2-carboxamido)picolinamide

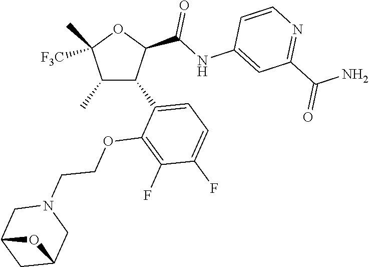 [SMILES: C[C@H]1[C@@H](c2ccc(F)c(F)c2OCCN2C[C@H]3C[C@@H](C2)O3)[C@H](C(=O)Nc2ccnc(C(N)=O)c2)O[C@@]1(C)C(F)(F)F]

4-((2R,3S,4S,5R)-3-(2-(2-((1R,5S)-6-oxa-3-azabicyclo
[3.1.1]heptan-3-yl)ethoxy)-3,4-difluorophenyl)-4,5-
dimethyl-5-(trifluoromethyl)tetrahydrofuran-2-carbox-
amido)picolinamide

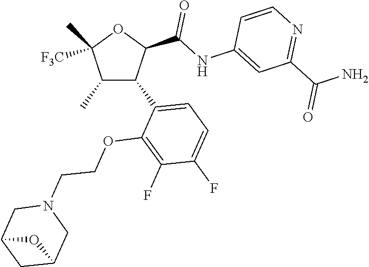 [SMILES: C[C@H]1[C@@H](c2ccc(F)c(F)c2OCCN2C[C@H]3C[C@@H](C2)O3)[C@H](C(=O)Nc2ccnc(C(N)=O)c2)O[C@@]1(C)C(F)(F)F]

4-((2R,3S,4S,5R)-3-(2-(2-((1S,5R)-6-oxa-3-azabicyclo
[3.1.1]heptan-3-yl)ethoxy)-3,4-difluorophenyl)-4,5-dimeth-
yl-5-(trifluoromethyl)tetrahydrofuran-2-carboxamido)-
picolinamide

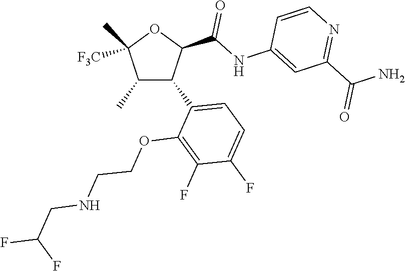 [SMILES: C[C@H]1[C@@H](c2ccc(F)c(F)c2OCCNCC(F)F)[C@H](C(=O)Nc2ccnc(C(N)=O)c2)O[C@@]1(C)C(F)(F)F]

4-((2R,3S,4S,5R)-3-(2-(2-((2,2-difluoroethyl)amino)ethoxy)-3,4-
difluorophenyl)-4,5-dimethyl-5-(trifluoromethyl)tetrahydrofuran-
2-carboxamido)picolinamide

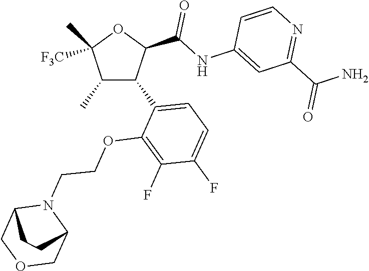 [SMILES: C[C@H]1[C@@H](c2ccc(F)c(F)c2OCCN2[C@@H]3CC[C@H]2COC3)[C@H](C(=O)Nc2ccnc(C(N)=O)c2)O[C@@]1(C)C(F)(F)F]

4-((2R,3S,4S,5R)-3-(2-(2-((1R,5S)-3-oxa-8-azabicyclo
[3.2.1]octan-8-yl)ethoxy)-3,4-difluorophenyl)-4,5-dimeth-
yl-5-(trifluoromethyl)tetrahydrofuran-2-carboxamido)-
picolinamide

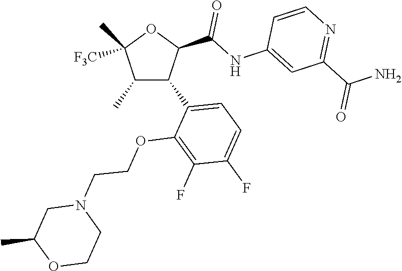 [SMILES: C[C@H]1CN(CCOc2c([C@H]3[C@H](C(=O)Nc4ccnc(C(N)=O)c4)O[C@@](C)(C(F)(F)F)[C@H]3C)ccc(F)c2F)CCO1]

4-((2R,3S,4S,5R)-3-(3,4-difluoro-2-(2-((S)-2-methylmorpholino)-
ethoxy)phenyl)-4,5-dimethyl-5-(trifluoromethyl)tetrahydrofuran-
2-carboxamido)picolinamide

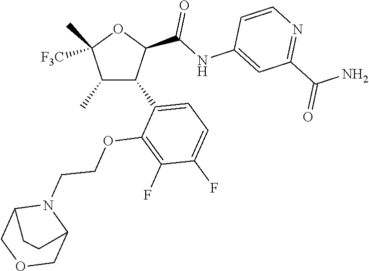 [SMILES: C[C@H]1[C@@H](c2ccc(F)c(F)c2OCCN2C3CCC2COC3)[C@H](C(=O)Nc2ccnc(C(N)=O)c2)O[C@@]1(C)C(F)(F)F]

4-((2R,3S,4S,5R)-3-(2-(2-(3-oxa-8-azabicyclo[3.2.1]octan-
8-yl)ethoxy)-3,4-difluorophenyl)-4,5-dimethyl-5-(trifluoro-
methyl)tetrahydrofuran-2-carboxamido)picolinamide

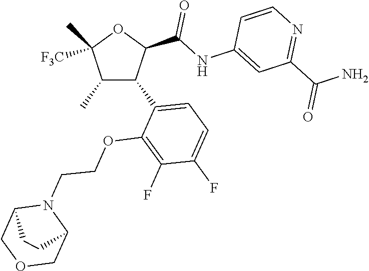 [SMILES: C[C@H]1[C@@H](c2ccc(F)c(F)c2OCCN2[C@@H]3CC[C@H]2COC3)[C@H](C(=O)Nc2ccnc(C(N)=O)c2)O[C@@]1(C)C(F)(F)F]

4-((2R,3S,4S,5R)-3-(2-(2-((1S,5R)-3-oxa-8-azabicyclo
[3.2.1]octan-8-yl)ethoxy)-3,4-difluorophenyl)-4,5-dimeth
yl-5-(trifluoromethyl)tetrahydrofuran-2-carboxamido)-
picolinamide

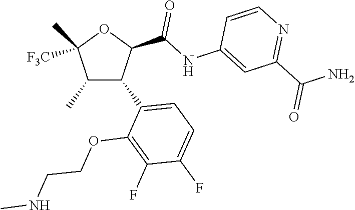 [SMILES: CNCCOc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(2-(methylamino)-
ethoxy)phenyl)-4,5-dimethyl-5-(trifluoromethyl)tetra-
hydrofuran-2-carboxamido)picolinamide

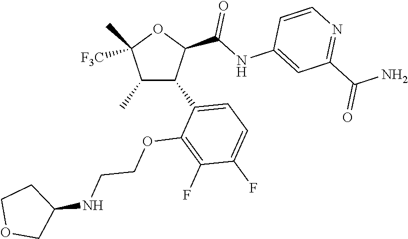 [SMILES: C[C@H]1[C@@H](c2ccc(F)c(F)c2OCCN[C@@H]2CCOC2)[C@H](C(=O)Nc2ccnc(C(N)=O)c2)O[C@@]1(C)C(F)(F)F]

4-((2R,3S,4S,5R)-3-(3,4-difluoro-2-(2-(((R)-tetrahydrofuran-3-yl)-
amino)ethoxy)phenyl)-4,5-dimethyl-5-trifluoromethyl)tetrahydro-
furan-(2-carboxamido)picolinamide

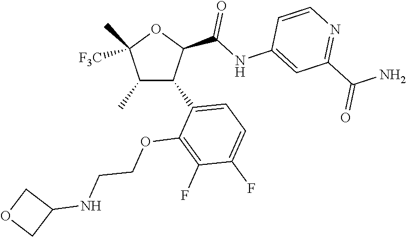 [SMILES: C[C@H]1[C@@H](c2ccc(F)c(F)c2OCCNC2COC2)[C@H](C(=O)Nc2ccnc(C(N)=O)c2)O[C@@]1(C)C(F)(F)F]

4-((2R,3S,4S,5R)-3-(3,4-difluoro-2-(2-(oxetan-3-ylamino)ethoxy)-
phenyl)-4,5-dimethyl-5-(trifluoromethyl)tetrahydrofuran-2-carbox-
amido)picolinamide

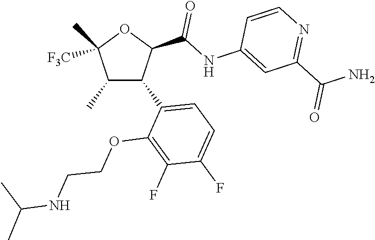 [SMILES: CC(C)NCCOc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(2-(isopropylamino)-
ethoxy)phenyl)-4,5-dimethyl-5-(trifluoromethyl)tetra-
hydrofuran-2-carboxamido)picolinamide

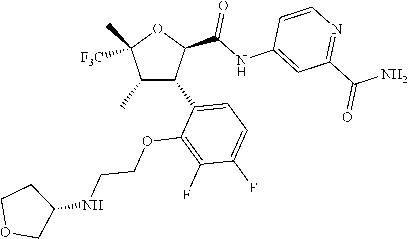 [SMILES: C[C@H]1[C@@H](c2ccc(F)c(F)c2OCCN[C@H]2CCOC2)[C@H](C(=O)Nc2ccnc(C(N)=O)c2)O[C@@]1(C)C(F)(F)F]

4-((2R,3S,4S,5R)-3-(3,4-difluoro-2-(2-(((S)-tetrahydrofuran-3-
yl)amino)ethoxy)phenyl)-4,5-dimethyl-5-(trifluoromethyl)tetra-
hydrofuran-2-carboxamido)picolinamide

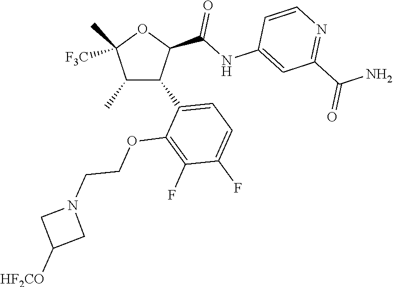

4-((2R,3S,4S,5R)-3-(2-(2-(3-(difluoromethoxy)azetidin-1-yl)-
ethoxy)-3,4-difluorophenyl)-4,5-dimethyl-5-(trifluoromethyl)-
tetrahydrofuran-2-carboxamido)picolinamide

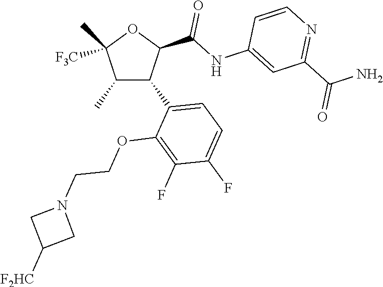 [SMILES: C[C@H]1[C@@H](c2ccc(F)c(F)c2OCCN2CC(C(F)F)C2)[C@H](C(=O)Nc2ccnc(C(N)=O)c2)O[C@@]1(C)C(F)(F)F]

4-((2R,3S,4S,5R)-3-(2-(2-(3-(difluoromethyl)azetidin-1-yl)-
ethoxy)-3,4-difluorophenyl)-4,5-dimethyl-5-(trifluorometh-
yl)tetrahydrofuran-2-carboxamido)picolinamide

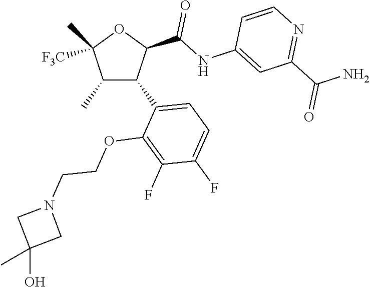 [SMILES: C[C@H]1[C@@H](c2ccc(F)c(F)c2OCCN2CC(C)(O)C2)[C@H](C(=O)Nc2ccnc(C(N)=O)c2)O[C@@]1(C)C(F)(F)F]

4-((2R,3S,4S,5R)-3-(3,4-difluoro-2-(2-(3-hydroxy-3-meth-
ylazetidin-1-yl)ethoxy)phenyl)-4,5-dimethyl-5-(trifluoro-
methyl)tetrahydrofuran-2-carboxamido)picolinamide

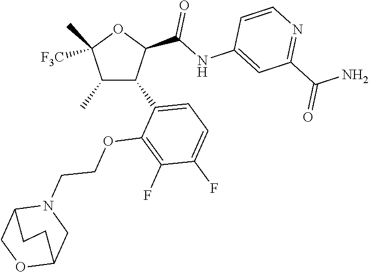 [SMILES: C[C@H]1[C@@H](c2ccc(F)c(F)c2OCCN2CC3CCC2CO3)[C@H](C(=O)Nc2ccnc(C(N)=O)c2)O[C@@]1(C)C(F)(F)F]

4-((2R,3S,4S,5R)-3-(2-(2-(2-oxa-5-azabicyclo[2.2.2]octan-
5-yl)ethoxy)-3,4-difluorophenyl)-4,5-dimethyl-5-(trifluoro-
methyl)tetrahydrofuran-2-carboxamido)picolinamide

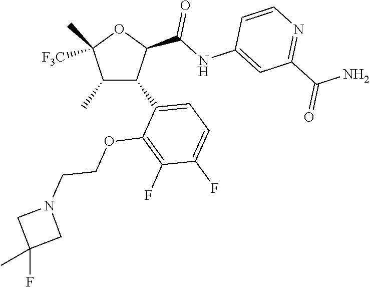 [SMILES: C[C@H]1[C@@H](c2ccc(F)c(F)c2OCCN2CC(C)(F)C2)[C@H](C(=O)Nc2ccnc(C(N)=O)c2)O[C@@]1(C)C(F)(F)F]

4-((2R,3S,4S,5R)-3-(3,4-difluoro-2-(2-(3-fluoro-3-methyl-
azetidin-1-yl)ethoxy)phenyl)-4,5-dimethyl-5-(trifluoro-
methyl)tetrahydrofuran-2-carboxamido)picolinamide

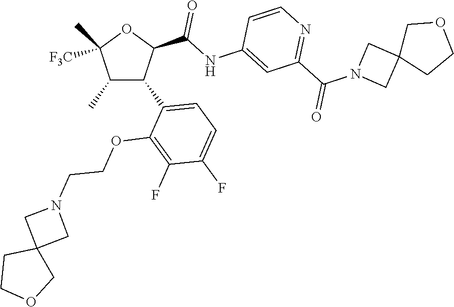 [SMILES: C[C@H]1[C@@H](c2ccc(F)c(F)c2OCCN2CC3(CCOC3)C2)[C@H](C(=O)Nc2ccnc(C(=O)N3CC4(CCOC4)C3)c2)O[C@@]1(C)C(F)(F)F]

(2R,3S,4S,5R)-3-(2-(2-(6-oxa-2-azaspiro[3.4]octan-2-yl)ethoxy)-3,4-
difluorophenyl)-N-(2-(6-oxa-2-azaspiro[3.4]octane-2-carbonyl)pyridin-
4-yl)-4,5-dimethyl-5-(trifluoromethyl)tetrahydrofuran-2-carboxamide

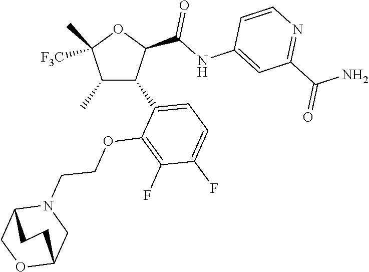 [SMILES: C[C@H]1[C@@H](c2ccc(F)c(F)c2OCCN2C[C@H]3CC[C@@H]2CO3)[C@H](C(=O)Nc2ccnc(C(N)=O)c2)O[C@@]1(C)C(F)(F)F]

4-((2R,3S,4S,5R)-3-(2-(2-((1R,4R)-2-oxa-5-azabicyclo
[2.2.2]octan-5-yl)ethoxy)-3,4-difluorophenyl)-4,5-
dimethyl-5-(trifluoromethyl)tetrahydrofuran-2-carbox-
amido)picolinamide

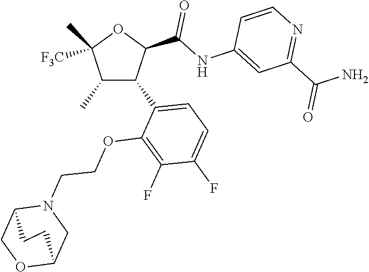 [SMILES: C[C@H]1[C@@H](c2ccc(F)c(F)c2OCCN2C[C@@H]3CC[C@H]2CO3)[C@H](C(=O)Nc2ccnc(C(N)=O)c2)O[C@@]1(C)C(F)(F)F]

4-((2R,3S,4S,5R)-3-(2-(2-((1S,4S)-2-oxa-5-azabicyclo-
[2.2.2]octan-5-yl)ethoxy)-3,4-difluorophenyl)-4,5-dimeth-
yl-5-(trifluoromethyl)tetrahydrofuran-2-carboxamido)-
picolinamide

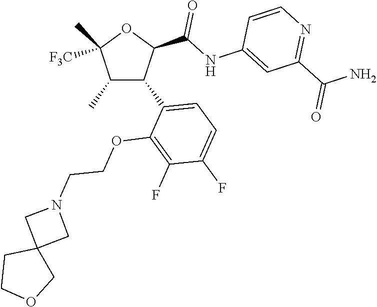 [SMILES: C[C@H]1[C@@H](c2ccc(F)c(F)c2OCCN2CC3(CCOC3)C2)[C@H](C(=O)Nc2ccnc(C(N)=O)c2)O[C@@]1(C)C(F)(F)F]

4-((2R,3S,4S,5R)-3-(2-(2-(6-oxa-2-azaspiro[3.4]octan-2-yl)-
ethoxy)-3,4-difluorophenyl)-4,5-dimethyl-5-(trifluorometh-
yl)tetrahydrofuran-2-carboxamido)picolinamide

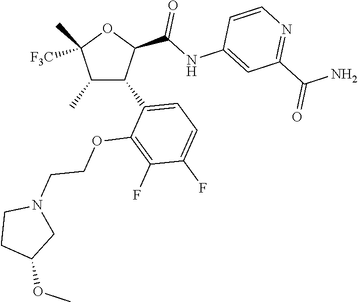 [SMILES: CO[C@@H]1CCN(CCOc2c([C@H]3[C@H](C(=O)Nc4ccnc(C(N)=O)c4)O[C@@](C)(C(F)(F)F)[C@H]3C)ccc(F)c2F)C1]

4-((2R,3S,4S,5R)-3-(3,4-difluoro-2-(2-((R)-3-methoxy-
pyrrolidin-1-yl)ethoxy)phenyl)-4,5-dimethyl-5-(trifluoro-
methyl)tetrahydrofuran-2-carboxamido)picolinamide

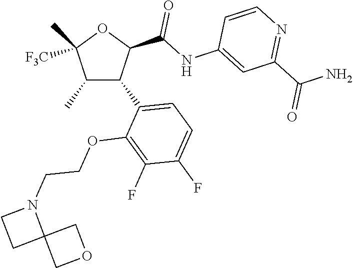 [SMILES: C[C@H]1[C@@H](c2ccc(F)c(F)c2OCCN2CCC23COC3)[C@H](C(=O)Nc2ccnc(C(N)=O)c2)O[C@@]1(C)C(F)(F)F]

4-((2R,3S,4S,5R)-3-(2-(2-(6-oxa-1-azaspiro[3.3]heptan-
1-yl)ethoxy)-3,4-difluorophenyl)-4,5-dimethyl-5-
(trifluoromethyl)tetrahydrofuran-2-carboxamido)-
picolinamide

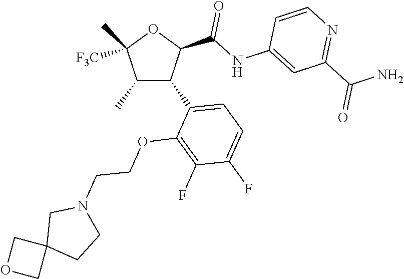 [SMILES: C[C@H]1[C@@H](c2ccc(F)c(F)c2OCCN2CCC3(COC3)C2)[C@H](C(=O)Nc2ccnc(C(N)=O)c2)O[C@@]1(C)C(F)(F)F]

4-((2R,3S,4S,5R)-3-(2-(2-(2-oxa-6-azaspiro[3.4]octan-6-yl)eth-
oxy)-3,4-difluorophenyl)-4,5-dimethyl-5-(trifluoromethyl)tetra-
hydrofuran-2-carboxamido)picolinamide

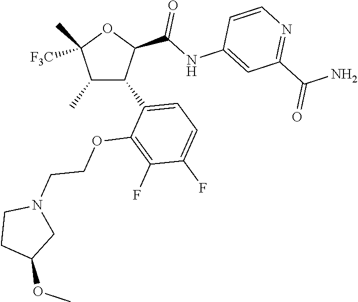 [SMILES: CO[C@H]1CCN(CCOc2c([C@H]3[C@H](C(=O)Nc4ccnc(C(N)=O)c4)O[C@@](C)(C(F)(F)F)[C@H]3C)ccc(F)c2F)C1]

4-((2R,3S,4S,5R)-3-(3,4-difluoro-2-(2-((S)-3-methoxy-
pyrrolidin-1-yl)ethoxy)phenyl)-4,5-dimethyl-5-(trifluoro-
methyl)tetrahydrofuran-2-carboxamido)picolinamide

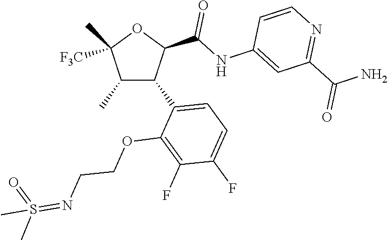 [SMILES: C[C@H]1[C@@H](c2ccc(F)c(F)c2OCCN=S(C)(C)=O)[C@H](C(=O)Nc2ccnc(C(N)=O)c2)O[C@@]1(C)C(F)(F)F]

4-((2R,3S,4S,5R)-3-(2-(2-((dimethyl(oxo)-1.6-sulfaneylidene)-
amino)ethoxy)-3,4-difluorophenyl)-4,5-dimethyl-5-(trifluoro-
methyl)tetrahydrofuran-2-carboxamido)picolinamide

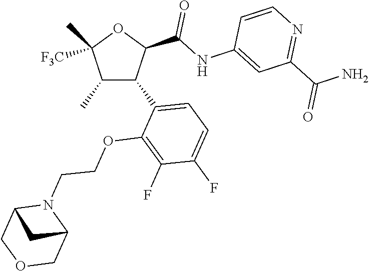 [SMILES: C[C@H]1[C@@H](c2ccc(F)c(F)c2OCCN2[C@@H]3COC[C@H]2C3)[C@H](C(=O)Nc2ccnc(C(N)=O)c2)O[C@@]1(C)C(F)(F)F]

4-((2R,3S,4S,5R)-3-(2-(2-((1R,5S)-3-oxa-6-azabicyclo
[3.1.1]heptan-6-yl)ethoxy)-3,4-difluorophenyl)-4,5-
dimethyl-5-(trifluoromethyl)tetrahydrofuran-2-carbox-
amido)picolinamide

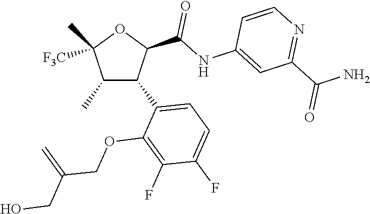 [SMILES: C=C(CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((2-(hydroxymethyl)-
allyl)oxy)phenyl)-4,5-dimethyl-5-(trifluoromethyl)tetra-
hydrofuran-2-carboxamido)picolinamide

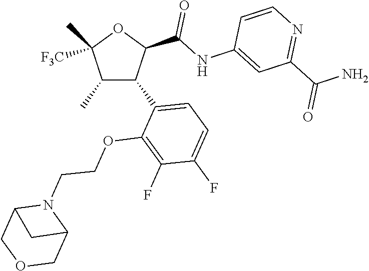 [SMILES: C[C@H]1[C@@H](c2ccc(F)c(F)c2OCCN2C3COCC2C3)[C@H](C(=O)Nc2ccnc(C(N)=O)c2)O[C@@]1(C)C(F)(F)F]

4-((2R,3S,4S,5R)-3-(2-(2-(3-oxa-6-azabicyclo[3.1.1]
heptan-6-yl)ethoxy)-3,4-difluorophenyl)-4,5-dimethyl-
5-(trifluoromethyl)tetrahydrofuran-2-carboxamido)-
picolinamide

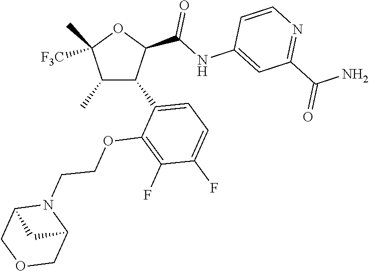 [SMILES: C[C@H]1[C@@H](c2ccc(F)c(F)c2OCCN2[C@@H]3COC[C@H]2C3)[C@H](C(=O)Nc2ccnc(C(N)=O)c2)O[C@@]1(C)C(F)(F)F]

4-((2R,3S,4S,5R)-3-(2-(2-((1S,5R)-3-oxa-6-azabicyclo
[3.1.1]heptan-6-yl)ethoxy)-3,4-difluorophenyl)-4,5-
dimethyl-5-(trifluoromethyl)tetrahydrofuran-2-carbox-
amido)picolinamide

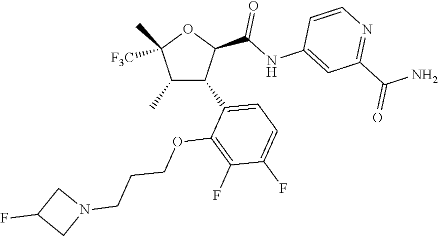 [SMILES: C[C@H]1[C@@H](c2ccc(F)c(F)c2OCCCN2CC(F)C2)[C@H](C(=O)Nc2ccnc(C(N)=O)c2)O[C@@]1(C)C(F)(F)F]

4-((2R,3S,4S,5R)-3-(3,4-difluoro-2-(3-(3-fluoroazetidin-1-yl)propoxy)-
phenyl)-4,5-dimethyl-5-(trifluoromethyl)tetrahydrofuran-2-carbox-
amido)picolinamide

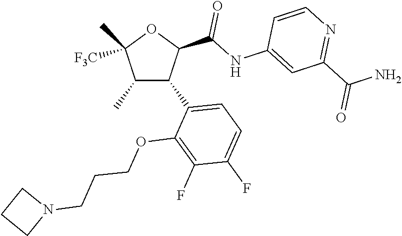 [SMILES: C[C@H]1[C@@H](c2ccc(F)c(F)c2OCCCN2CCC2)[C@H](C(=O)Nc2ccnc(C(N)=O)c2)O[C@@]1(C)C(F)(F)F]

4-((2R,3S,4S,5R)-3-(2-(3-(azetidin-1-yl)propoxy)-3,4-difluoro-
phenyl)-4,5-dimethyl-5-(trifluoromethyl)tetrahydrofuran-2-
carboxamido)picolinamide

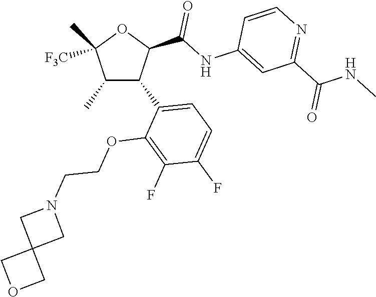 [SMILES: CNC(=O)c1cc(NC(=O)[C@@H]2O[C@@](C)(C(F)(F)F)[C@@H](C)[C@H]2c2ccc(F)c(F)c2OCCN2CC3(COC3)C2)ccn1]

4-((2R,3S,4S,5R)-3-(2-(2-(2-oxa-6-azaspiro[3.3]heptan-6-
yl)ethoxy)-3,4-difluorophenyl)-4,5-dimethyl-5-(trifluoro-
methyl)tetrahydrofuran-2-carboxamido)-N-methyl-
picolinamide

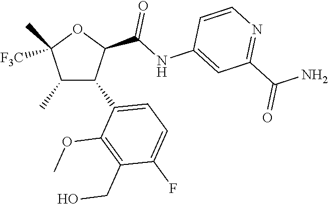 [SMILES: COc1c([C@H]2[C@H](C(=O)Nc3ccnc(C(N)=O)c3)O[C@@](C)(C(F)(F)F)[C@H]2C)ccc(F)c1CO]

4-((2R,3S,4S,5R)-3-(4-fluoro-3-(hydroxymethyl)-2-
methoxyphenyl)-4,5-dimethyl-5-(trifluoromethyl)-
tetrahydrofuran-2-carboxamido)picolinamide

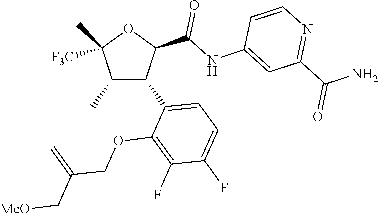 [SMILES: C=C(COC)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((2-(methoxymethyl)-
allyl)oxy)phenyl)-4,5-dimethyl-5-(trifluoromethyl)tetra-
hydrofuran-2-carboxamido)picolinamide

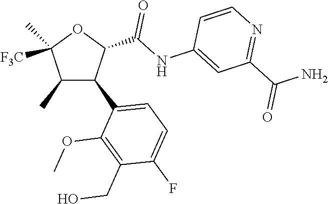 [SMILES: COc1c([C@@H]2[C@@H](C(=O)Nc3ccnc(C(N)=O)c3)O[C@](C)(C(F)(F)F)[C@@H]2C)ccc(F)c1CO]

4-((2S,3R,4R,5S)-3-(4-fluoro-3-(hydroxymethyl)-2-
methoxyphenyl)-4,5-dimethyl-5-(trifluoromethyl)
tetrahydrofuran-2-carboxamido)picolinamide

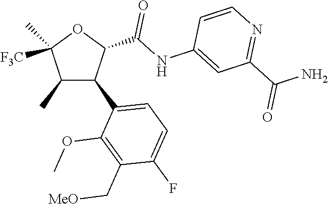 [SMILES: COCc1c(F)ccc([C@@H]2[C@@H](C(=O)Nc3ccnc(C(N)=O)c3)O[C@](C)(C(F)(F)F)[C@@H]2C)c1OC]

4-((2S,3R,4R,5S)-3-(4-fluoro-2-methoxy-3-(meth-
oxymethyl)phenyl)-4,5-dimethyl-5-(trifluorometh-
yl)tetrahydrofuran-2-carboxamido)picolinamide

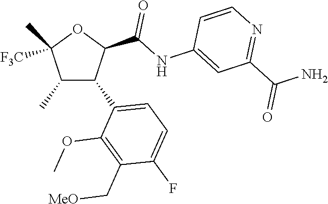 [SMILES: COCc1c(F)ccc([C@H]2[C@H](C(=O)Nc3ccnc(C(N)=O)c3)O[C@@](C)(C(F)(F)F)[C@H]2C)c1OC]

4-((2R,3S,4S,5R)-3-(4-fluoro-2-methoxy-3-(meth-
oxymethyl)phenyl)-4,5-dimethyl-5-trifluoromethyl)-
tetrahydrofuran-(2-carboxamido)picolinamide

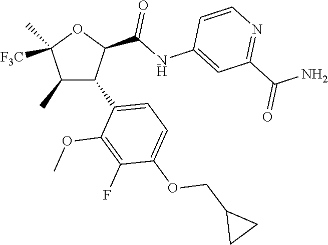 [SMILES: COc1c([C@@H]2[C@@H](C)[C@@](C)(C(F)(F)F)O[C@H]2C(=O)Nc2ccnc(C(N)=O)c2)ccc(OCC2CC2)c1F]

4-((2R,3S,4R,5S)-3-(4-(cyclopropylmethoxy)-3-
fluoro-2-methoxyphenyl)-4,5-dimethyl-5-(trifluoro-
methyl)tetrahydrofuran-2-carboxamido)picolinamide

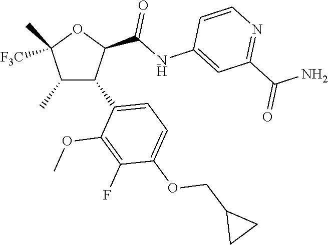 [SMILES: COc1c([C@H]2[C@H](C(=O)Nc3ccnc(C(N)=O)c3)O[C@@](C)(C(F)(F)F)[C@H]2C)ccc(OCC2CC2)c1F]

4-((2R,3S,4S,5R)-3-(4-(cyclopropylmethoxy)-3-
fluoro-2-methoxyphenyl)-4,5-dimethyl-5-(trifluoro-
methyl)tetrahydrofuran-2-carboxamido)picolinamide

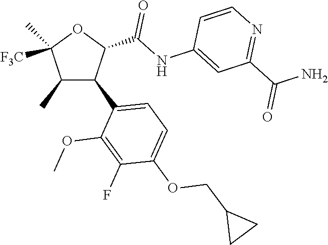 [SMILES: COc1c([C@@H]2[C@@H](C(=O)Nc3ccnc(C(N)=O)c3)O[C@](C)(C(F)(F)F)[C@@H]2C)ccc(OCC2CC2)c1F]

4-((2S,3R,4R,5S)-3-(4-(cyclopropylmethoxy)-3-
fluoro-2-methoxyphenyl)-4,5-dimethyl-5-(trifluoro-
methyl)tetrahydrofuran-2-carboxamido)picolinamide

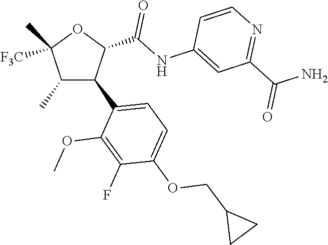 [SMILES: COc1c([C@@H]2[C@@H](C(=O)Nc3ccnc(C(N)=O)c3)O[C@@](C)(C(F)(F)F)[C@H]2C)ccc(OCC2CC2)c1F]

4-((2S,3R,4S,5R)-3-(4-(cyclopropylmethoxy)-3-
fluoro-2-methoxyphenyl)-4,5-dimethyl-5-(trifluoro-
methyl)tetrahydrofuran-2-carboxamido)picolinamide

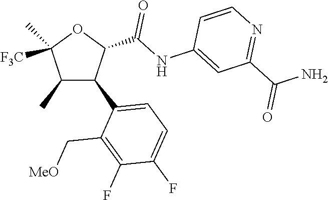 [SMILES: COCc1c([C@@H]2[C@@H](C(=O)Nc3ccnc(C(N)=O)c3)O[C@](C)(C(F)(F)F)[C@@H]2C)ccc(F)c1F]

4-((2S,3R,4R,5S)-3-(3,4-difluoro-2-(methoxymeth-
yl)phenyl)-4,5-dimethyl-5-(trifluoromethyl)tetra-
hydrofuran-2-carboxamido)picolinamide

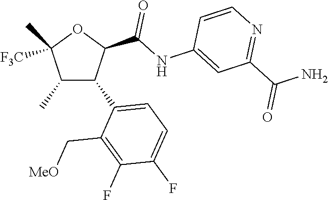 [SMILES: COCc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(methoxymeth-
yl)phenyl)-4,5-dimethyl-5-(trifluoromethyl)tetra-
hydrofuran-2-carboxamido)picolinamide

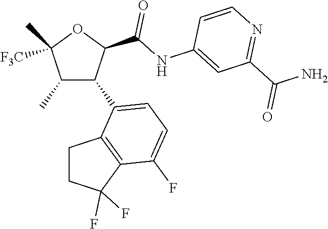 [SMILES: C[C@H]1[C@@H](c2ccc(F)c3c2CCC3(F)F)[C@H](C(=O)Nc2ccnc(C(N)=O)c2)O[C@@]1(C)C(F)(F)F]

4-((2R,3S,4S,5R)-4,5-dimethyl-3-(1,1,7-trifluoro-
2,3-dihydro-1H-inden-4-yl)-5-(trifluoromethyl)tetra-
hydrofuran-2-carboxamido)picolinamide

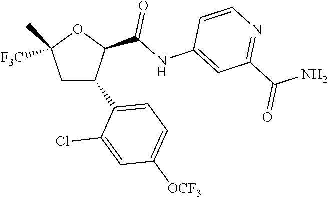 [SMILES: C[C@]1(C(F)(F)F)C[C@@H](c2ccc(OC(F)(F)F)cc2Cl)[C@H](C(=O)Nc2ccnc(C(N)=O)c2)O1]

4-((2R,3S,5R)-3-(2-chloro-4-(trifluoromethoxy)-
phenyl)-5-methyl-5-(trifluoromethyl)tetrahydro-
furan-2-carboxamido)picolinamide

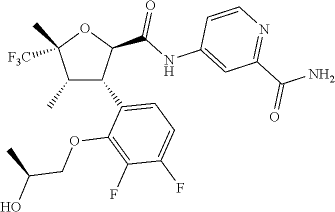 [SMILES: C[C@H](O)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((S)-2-hydroxy-
propoxy)phenyl)-4,5-dimethyl-5-(trifluoromethyl)-
tetrahydrofuran-2-carboxamido)picolinamide

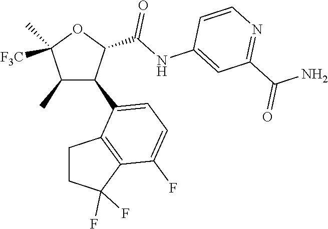 [SMILES: C[C@@H]1[C@H](c2ccc(F)c3c2CCC3(F)F)[C@@H](C(=O)Nc2ccnc(C(N)=O)c2)O[C@]1(C)C(F)(F)F]

4-((2S,3R,4R,5S)-4,5-dimethyl-3-(1,1,7-trifluoro-
2,3-dihydro-1H-inden-4-yl)-5-(trifluoromethyl)tetra-
hydrofuran-2-carboxamido)picolinamide

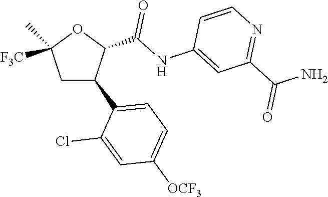 [SMILES: C[C@@]1(C(F)(F)F)C[C@H](c2ccc(OC(F)(F)F)cc2Cl)[C@@H](C(=O)Nc2ccnc(C(N)=O)c2)O1]

4-((2S,3R,5S)-3-(2-chloro-4-(trifluoromethoxy)phen-
yl)-5-methyl-5-(trifluoromethyl)tetrahydrofuran-2-
carboxamido)picolinamide

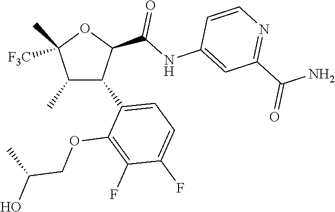 [SMILES: C[C@@H](O)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((R)-2-hydroxy-
propoxy)phenyl)-4,5-dimethyl-5-trifluoromethyl)-
(tetrahydrofuran-2-carboxamido)picolinamide

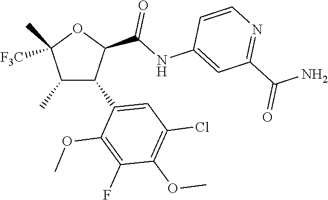 [SMILES: COc1c(Cl)cc([C@H]2[C@H](C(=O)Nc3ccnc(C(N)=O)c3)O[C@@](C)(C(F)(F)F)[C@H]2C)c(OC)c1F]

4-((2R,3S,4S,5R)-3-(5-chloro-3-fluoro-2,4-dimeth-
oxyphenyl)-4,5-dimethyl-5-(trifluoromethyl)tetra-
hydrofuran-2-carboxamido)picolinamide

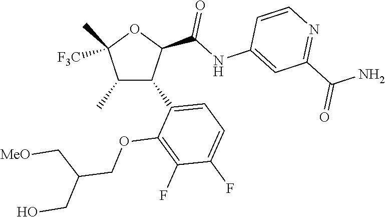 [SMILES: COCC(CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(3-hydroxy-2-(methoxy-
methyl)propoxy)phenyl)-4,5-dimethyl-5-(trifluoromethyl)-
tetrahydrofuran-2-carboxamido)picolinamide

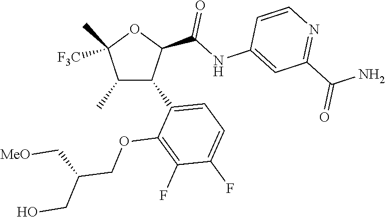 [SMILES: COC[C@@H](CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((R)-3-hydroxy-2-(meth-
oxymethyl)propoxy)phenyl)-4,5-dimethyl-5-(trifluoromethyl)-
tetrahydrofuran-2-carboxamido)picolinamide

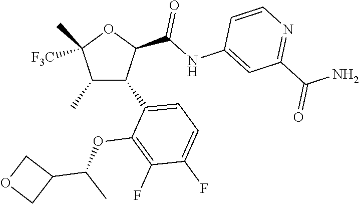 [SMILES: C[C@@H](Oc1c([C@H]2[C@H](C(=O)Nc3ccnc(C(N)=O)c3)O[C@@](C)(C(F)(F)F)[C@H]2C)ccc(F)c1F)C1COC1]

4-((2R,3S,4S,5R)-3-(3,4-difluoro-2-((R)-1-(oxetan-3-yl)-
ethoxy)phenyl)-4,5-dimethyl-5-(trifluoromethyl)tetra-
hydrofuran-2-carboxamido)picolinamide

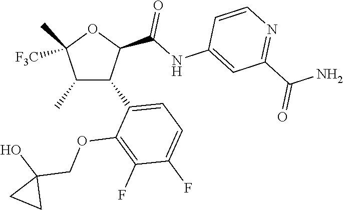 [SMILES: C[C@H]1[C@@H](c2ccc(F)c(F)c2OCC2(O)CC2)[C@H](C(=O)Nc2ccnc(C(N)=O)c2)O[C@@]1(C)C(F)(F)F]

4-((2R,3S,4S,5R)-3-(3,4-difluoro-2-((1-hydroxycyclo-
propyl)methoxy)phenyl)-4,5-dimethyl-5-(trifluorometh-
yl)tetrahydrofuran-2-carboxamido)picolinamide

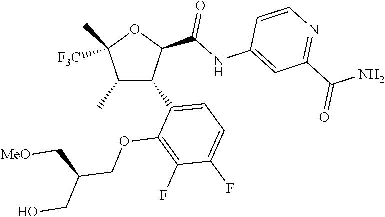 [SMILES: COC[C@H](CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((S)-3-hydroxy-2-(meth-
oxymethyl)propoxy)phenyl)-4,5-dimethyl-5-(trifluorometh-
yl)tetrahydrofuran-2-carboxamido)picolinamide

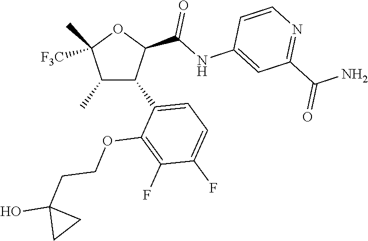 [SMILES: C[C@H]1[C@@H](c2ccc(F)c(F)c2OCCC2(O)CC2)[C@H](C(=O)Nc2ccnc(C(N)=O)c2)O[C@@]1(C)C(F)(F)F]

4-((2R,3S,4S,5R)-3-(3,4-difluoro-2-(2-(1-hydroxycyclo-
propyl)ethoxy)phenyl)-4,5-dimethyl-5-(trifluoromethyl)-
tetrahydrofuran-2-carboxamido)picolinamide

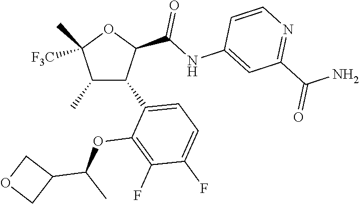 [SMILES: C[C@H](Oc1c([C@H]2[C@H](C(=O)Nc3ccnc(C(N)=O)c3)O[C@@](C)(C(F)(F)F)[C@H]2C)ccc(F)c1F)C1COC1]

4-((2R,3S,4S,5R)-3-(3,4-difluoro-2-((S)-1-(oxetan-3-yl)-
ethoxy)phenyl)-4,5-dimethyl-5-(trifluoromethyl)tetra-
hydrofuran-2-carboxamido)picolinamide

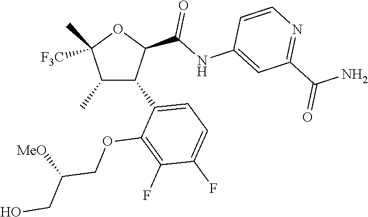 [SMILES: CO[C@@H](CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((S)-3-hydroxy-2-meth-
oxypropoxy)phenyl)-4,5-dimethyl-5-(trifluoromethyl)tetra-
hydrofuran-2-carboxamido)picolinamide

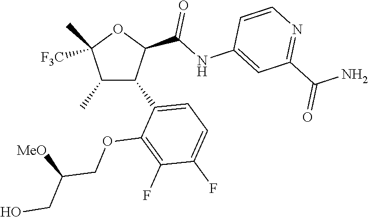 [SMILES: CO[C@H](CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((R)-3- hydroxy-2-meth-
oxypropoxy)phenyl)-4,5-dimethyl-5-(trifluoromethyl)tetra-
hydrofuran-2-carboxamido)picolinamide

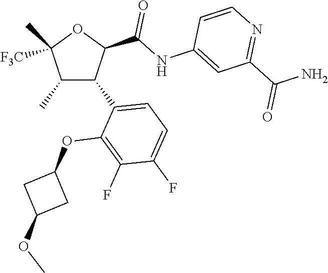 [SMILES: CO[C@H]1C[C@@H](Oc2c([C@H]3[C@H](C(=O)Nc4ccnc(C(N)=O)c4)O[C@@](C)(C(F)(F)F)[C@H]3C)ccc(F)c2F)C1]

4-((2R,3S,4S,5R)-3-(3,4-difluoro-2-((1s,3R)-3-meth-
oxycyclobutoxy)phenyl)-4,5-dimethyl-5-(trifluoro-
methyl)tetrahydrofuran-2-carboxamido)picolinamide

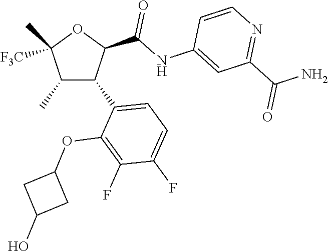 [SMILES: C[C@H]1[C@@H](c2ccc(F)c(F)c2OC2CC(O)C2)[C@H](C(=O)Nc2ccnc(C(N)=O)c2)O[C@@]1(C)C(F)(F)F]

4-((2R,3S,4S,5R)-3-(3,4-difluoro-2-(3-hydroxycyclo-
butoxy)phenyl)-4,5-dimethyl-5-(trifluoromethyl)tetra-
hydrofuran-2-carboxamido)picolinamide

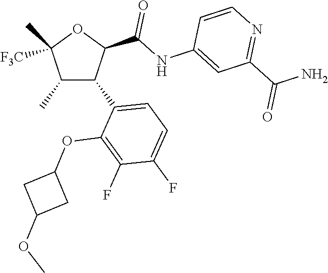 [SMILES: COC1CC(Oc2c([C@H]3[C@H](C(=O)Nc4ccnc(C(N)=O)c4)O[C@@](C)(C(F)(F)F)[C@H]3C)ccc(F)c2F)C1]

4-((2R,3S,4S,5R)-3-(3,4-difluoro-2-(3-methoxycyclo-
butoxy)phenyl)-4,5-dimethyl-5-(trifluoromethyl)-
tetrahydrofuran-2-carboxamido)picolinamide

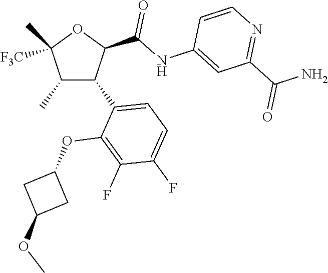 [SMILES: CO[C@H]1C[C@H](Oc2c([C@H]3[C@H](C(=O)Nc4ccnc(C(N)=O)c4)O[C@@](C)(C(F)(F)F)[C@H]3C)ccc(F)c2F)C1]

4-((2R,3S,4S,5R)-3-(3,4-difluoro-2-((1r,3S)-3-meth-
oxycyclobutoxy)phenyl)-4,5-dimethyl-5-(trifluoro-
methyl)tetrahydrofuran-2-carboxamido)picolinamide

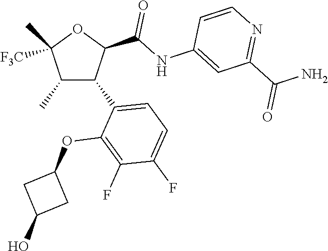 [SMILES: C[C@H]1[C@@H](c2ccc(F)c(F)c2O[C@H]2C[C@@H](O)C2)[C@H](C(=O)Nc2ccnc(C(N)=O)c2)O[C@@]1(C)C(F)(F)F]

4-((2R,3S,4S,5R)-3-(3,4-difluoro-2-((1s,3R)-3-
hydroxycyclobutoxy)phenyl)-4,5-dimethyl-5-
(trifluoromethyl)tetrahydrofuran-2-carbox-
amido)picolinamide

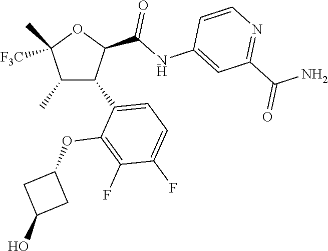 [SMILES: C[C@H]1[C@@H](c2ccc(F)c(F)c2O[C@H]2C[C@H](O)C2)[C@H](C(=O)Nc2ccnc(C(N)=O)c2)O[C@@]1(C)C(F)(F)F]

4-((2R,3S,4S,5R)-3-(3,4-difluoro-2-((1r,3S)-3-
hydroxycyclobutoxy)phenyl)-4,5-dimethyl-5-
(trifluoromethyl)tetrahydrofuran-2-carbox-
amido)picolinamide

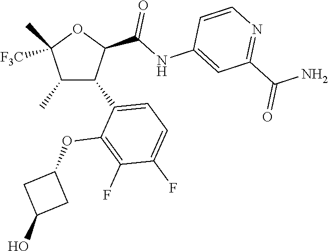 [SMILES: C[C@H]1[C@@H](c2ccc(F)c(F)c2O[C@H]2C[C@H](O)C2)[C@H](C(=O)Nc2ccnc(C(N)=O)c2)O[C@@]1(C)C(F)(F)F]

4-((2R,3S,4S,5R)-3-(3,4-difluoro-2-(((E)-2-(hydroxy-
methyl)but-2-en-1-yl)oxy)phenyl)-4,5-dimethyl-5-
(trifluoromethyl)tetrahydrofuran-2-carboxamido)-
picolinamide

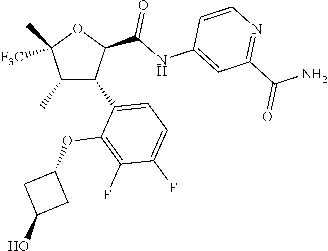 [SMILES: C[C@H]1[C@@H](c2ccc(F)c(F)c2O[C@H]2C[C@H](O)C2)[C@H](C(=O)Nc2ccnc(C(N)=O)c2)O[C@@]1(C)C(F)(F)F]

4-((2R,3S,4S,5R)-3-(3,4-difluoro-2-((1r,3S)-3-
hydroxycyclobutoxy)phenyl)-4,5-dimethyl-5-(tri-
fluoromethyl)tetrahydrofuran-2-carboxamido)-
picolinamide

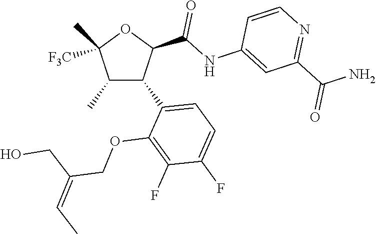 [SMILES: C/C=C(/CO)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(((Z)-2-(hydroxymethyl)-
but-2-en-1-yl)oxy)phenyl)-4,5-dimethyl-5-(trifluoromethyl)
tetrahydrofuran-2-carboxamido)picolinamide

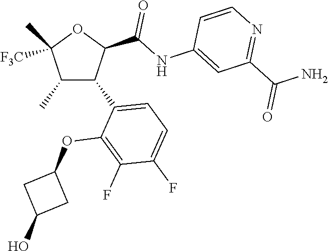 [SMILES: C[C@H]1[C@@H](c2ccc(F)c(F)c2O[C@H]2C[C@@H](O)C2)[C@H](C(=O)Nc2ccnc(C(N)=O)c2)O[C@@]1(C)C(F)(F)F]

4-((2R,3S,4S,5R)-3-(3,4-difluoro-2-((1s,3R)-3-
hydroxycyclobutoxy)phenyl)-4,5-dimethyl-5-
(trifluoromethyl)tetrahydrofuran-2-carbox-
amido)picolinamide

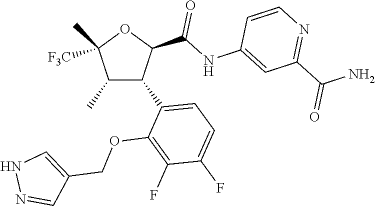 [SMILES: C[C@H]1[C@@H](c2ccc(F)c(F)c2OCc2cn[nH]c2)[C@H](C(=O)Nc2ccnc(C(N)=O)c2)O[C@@]1(C)C(F)(F)F]

4-((2R,3S,4S,5R)-3-(2-((1H-pyrazol-4-yl)methoxy)-3,4-
difluorophenyl)-4,5-dimethyl-5-(trifluoromethyl)tetra-
hydrofuran-2-carboxamido)picolinamide

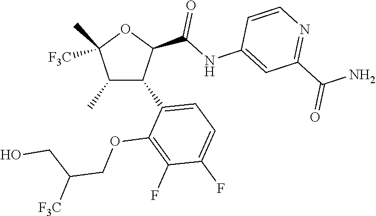 [SMILES: C[C@H]1[C@@H](c2ccc(F)c(F)c2OCC(CO)C(F)(F)F)[C@H](C(=O)Nc2ccnc(C(N)=O)c2)O[C@@]1(C)C(F)(F)F]

4-((2R,3S,4S,5R)-3-(3,4-difluoro-2-(3,3,3-trifluoro-2-
(hydroxymethyl)propoxy)phenyl)-4,5-dimethyl-5-
(trifluoromethyl)tetrahydrofuran-2-carboxamido)-
picolinamide

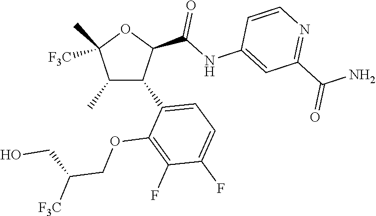 [SMILES: C[C@H]1[C@@H](c2ccc(F)c(F)c2OC[C@@H](CO)C(F)(F)F)[C@H](C(=O)Nc2ccnc(C(N)=O)c2)O[C@@]1(C)C(F)(F)F]

4-((2R,3S,4S,5R)-3-(3,4-difluoro-2-((R)-3,3,3-trifluoro-2-
(hydroxymethyl)propoxy)phenyl)-4,5-dimethyl-5-(trifluoro-
methyl)tetrahydrofuran-2-carboxamido)picolinamide

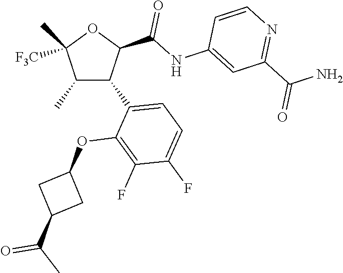 [SMILES: CC(=O)[C@H]1C[C@@H](Oc2c([C@H]3[C@H](C(=O)Nc4ccnc(C(N)=O)c4)O[C@@](C)(C(F)(F)F)[C@H]3C)ccc(F)c2F)C1]

4-((2R,3S,4S,5R)-3-(2-((1s,3R)-3-acetylcyclobutoxy)-
3,4-difluorophenyl)-4,5-dimethyl-5-(trifluoromethyl)-
tetrahydrofuran-2-carboxamido)picolinamide

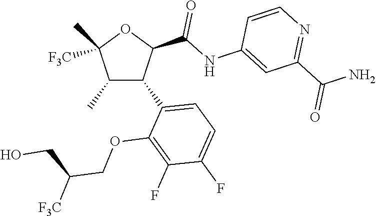 [SMILES: C[C@H]1[C@@H](c2ccc(F)c(F)c2OC[C@H](CO)C(F)(F)F)[C@H](C(=O)Nc2ccnc(C(N)=O)c2)O[C@@]1(C)C(F)(F)F]

4-((2R,3S,4S,5R)-3-(3,4-difluoro-2-((S)-3,3,3-trifluoro-2-
(hydroxymethyl)propoxy)phenyl)-4,5-dimethyl-5-(trifluoro-
methyl)tetrahydrofuran-2-carboxamido)picolinamide

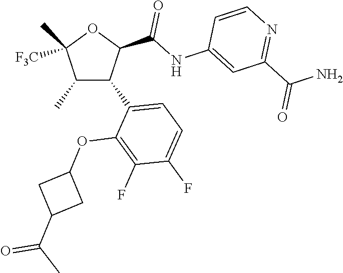 [SMILES: CC(=O)C1CC(Oc2c([C@H]3[C@H](C(=O)Nc4ccnc(C(N)=O)c4)O[C@@](C)(C(F)(F)F)[C@H]3C)ccc(F)c2F)C1]

4-((2R,3S,4S,5R)-3-(2-(3-acetylcyclobutoxy)-3,4-
difluorophenyl)-4,5-dimethyl-5-(trifluoromethyl)-
tetrahydrofuran-2-carboxamido)picolinamide

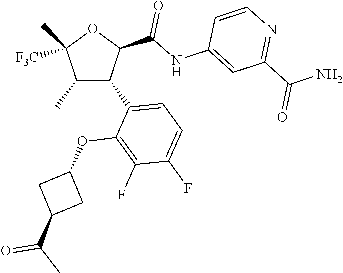 [SMILES: CC(=O)[C@H]1C[C@H](Oc2c([C@H]3[C@H](C(=O)Nc4ccnc(C(N)=O)c4)O[C@@](C)(C(F)(F)F)[C@H]3C)ccc(F)c2F)C1]

4-((2R,3S,4S,5R)-3-(2-((1r,3S)-3-acetylcyclobutoxy)-
3,4-difluorophenyl)-4,5-dimethyl-5-(trifluoromethyl)-
tetrahydrofuran-2-carboxamido)picolinamide

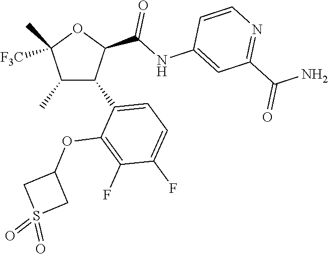 [SMILES: C[C@H]1[C@@H](c2ccc(F)c(F)c2OC2CS(=O)(=O)C2)[C@H](C(=O)Nc2ccnc(C(N)=O)c2)O[C@@]1(C)C(F)(F)F]

4-((2R,3S,4S,5R)-3-(2-((1,1-dioxidothietan-3-yl)-
oxy)-3,4-difluorophenyl)-4,5-dimethyl-5-
(trifluoromethyl)tetrahydrofuran-2-carbox-
amido)picolinamide

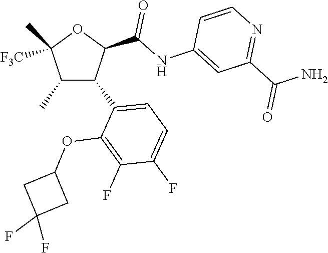 [SMILES: C[C@H]1[C@@H](c2ccc(F)c(F)c2OC2CC(F)(F)C2)[C@H](C(=O)Nc2ccnc(C(N)=O)c2)O[C@@]1(C)C(F)(F)F]

4-((2R,3S,4S,5R)-3-(2-(3,3-difluorocyclobutoxy)-
3,4-difluorophenyl)-4,5-dimethyl-5-(trifluorometh-
yl)tetrahydrofuran-2-carboxamido)picolinamide

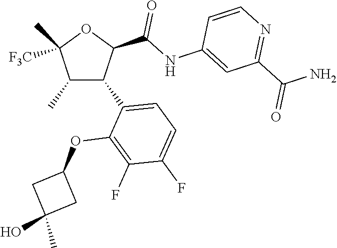 [SMILES: C[C@H]1[C@@H](c2ccc(F)c(F)c2O[C@H]2C[C@@](C)(O)C2)[C@H](C(=O)Nc2ccnc(C(N)=O)c2)O[C@@]1(C)C(F)(F)F]

4-((2R,3S,4S,5R)-3-(3,4-difluoro-2-((1s,3R)-3-
hydroxy-3-methylcyclobutoxy)phenyl)-4,5-
dimethyl-5-(trifluoromethyl)tetrahydrofuran-2-
carboxamido)picolinamide

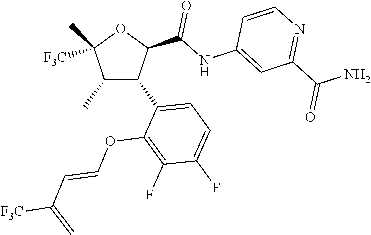 [SMILES: C=C(/C=C/Oc1c([C@H]2[C@H](C(=O)Nc3ccnc(C(N)=O)c3)O[C@@](C)(C(F)(F)F)[C@H]2C)ccc(F)c1F)C(F)(F)F]

4-((2R,3S,4S,5R)-3-(3,4-difluoro-2-(((E)-3-(trifluorometh-
yl)buta-1,3-dien-1-yl)oxy)phenyl)-4,5-dimethyl-5-(trifluoro-
methyl)tetrahydrofuran-2-carboxamido)picolinamide

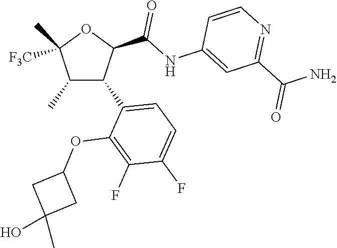 [SMILES: C[C@H]1[C@@H](c2ccc(F)c(F)c2OC2CC(C)(O)C2)[C@H](C(=O)Nc2ccnc(C(N)=O)c2)O[C@@]1(C)C(F)(F)F]

4-((2R,3S,4S,5R)-3-(3,4-difluoro-2-(3-hydroxy-3-
methylcyclobutoxy)phenyl)-4,5-dimethyl-5-
(trifluoromethyl)tetrahydrofuran-2-carboxamido)-
picolinamide

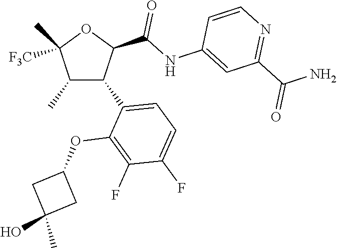 [SMILES: C[C@H]1[C@@H](c2ccc(F)c(F)c2O[C@H]2C[C@](C)(O)C2)[C@H](C(=O)Nc2ccnc(C(N)=O)c2)O[C@@]1(C)C(F)(F)F]

4-((2R,3S,4S,5R)-3-(3,4-difluoro-2-((1r,3S)-3-
hydroxy-3-methylcyclobutoxy)phenyl)-4,5-dimethyl-
5-(trifluoro-methyl)tetrahydrofuran-2-carboxamido)-
picolinamide

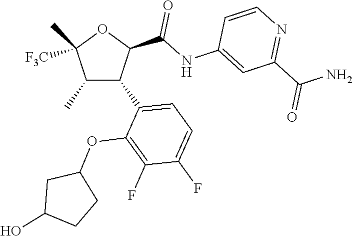 [SMILES: C[C@H]1[C@@H](c2ccc(F)c(F)c2OC2CCC(O)C2)[C@H](C(=O)Nc2ccnc(C(N)=O)c2)O[C@@]1(C)C(F)(F)F]

4-((2R,3S,4S,5R)-3-(3,4-difluoro-2-((3-hydroxycyclo-
pentyl)oxy)phenyl)-4,5-dimethyl-5-(trifluoromethyl)-
tetrahydrofuran-2-carboxamido)picolinamide

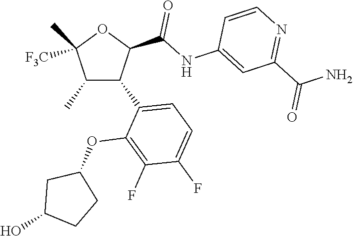 [SMILES: C[C@H]1[C@@H](c2ccc(F)c(F)c2O[C@@H]2CC[C@H](O)C2)[C@H](C(=O)Nc2ccnc(C(N)=O)c2)O[C@@]1(C)C(F)(F)F]

4-((2R,3S,4S,5R)-3-(3,4-difluoro-2-(((1R,3S)-3-hydroxy-
cyclopentyl)oxy)phenyl)-4,5-dimethyl-5-(trifluorometh-
yl)tetrahydrofuran-2-carboxamido)picolinamide

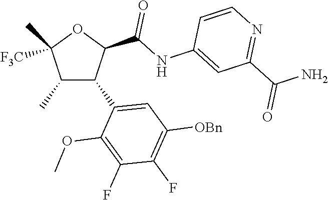 [SMILES: COc1c([C@H]2[C@H](C(=O)Nc3ccnc(C(N)=O)c3)O[C@@](C)(C(F)(F)F)[C@H]2C)cc(OCc2ccccc2)c(F)c1F]

4-((2R,3S,4S,5R)-3-(5-(benzyloxy)-3,4-difluoro-2-
methoxyphenyl)-4,5-dimethyl-5-(trifluoromethyl)-
tetrahydrofuran-2-carboxamido)picolinamide

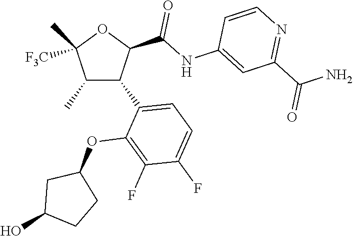 [SMILES: C[C@H]1[C@@H](c2ccc(F)c(F)c2O[C@H]2CC[C@@H](O)C2)[C@H](C(=O)Nc2ccnc(C(N)=O)c2)O[C@@]1(C)C(F)(F)F]

4-((2R,3S,4S,5R)-3-(3,4-difluoro-2-(((1S,3R)-3-hydroxy-
cyclopentyl)oxy)phenyl)-4,5-dimethyl-5-(trifluorometh-
yl)tetrahydrofuran-2-carboxamido)picolinamide

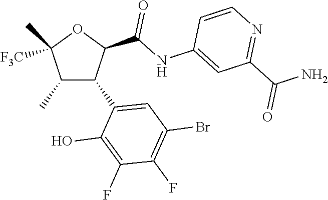 [SMILES: C[C@H]1[C@@H](c2cc(Br)c(F)c(F)c2O)[C@H](C(=O)Nc2ccnc(C(N)=O)c2)O[C@@]1(C)C(F)(F)F]

4-((2R,3S,4S,5R)-3-(5-bromo-3,4-difluoro-2-
hydroxyphenyl)-4,5-dimethyl-5-(trifluorometh-
yl)tetrahydrofuran-2-carboxamido)picolinamide

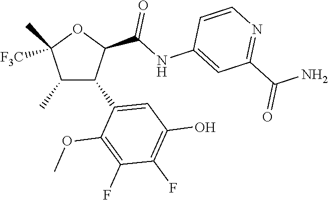 [SMILES: COc1c([C@H]2[C@H](C(=O)Nc3ccnc(C(N)=O)c3)O[C@@](C)(C(F)(F)F)[C@H]2C)cc(O)c(F)c1F]

4-((2R,3S,4S,5R)-3-(3,4-difluoro-5-hydroxy-2-
methoxyphenyl)-4,5-dimethyl-5-(trifluoromethyl)-
tetrahydrofuran-2-carboxamido)picolinamide

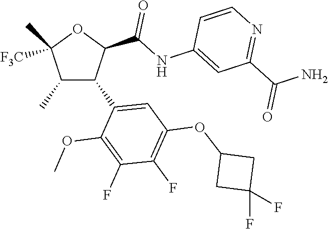 [SMILES: COc1c([C@H]2[C@H](C(=O)Nc3ccnc(C(N)=O)c3)O[C@@](C)(C(F)(F)F)[C@H]2C)cc(OC2CC(F)(F)C2)c(F)c1F]

4-((2R,3S,4S,5R)-3-(5-(3,3-difluorocyclobutoxy)-
3,4-difluoro-2-methoxyphenyl)-4,5-dimethyl-5-
(trifluoromethyl)tetrahydrofuran-2-carboxamido)-
picolinamide

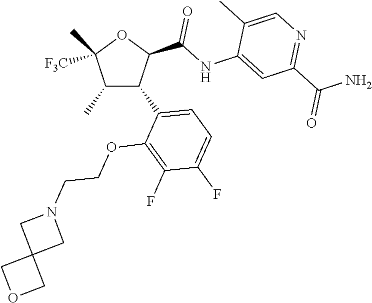 [SMILES: Cc1cnc(C(N)=O)cc1NC(=O)[C@@H]1O[C@@](C)(C(F)(F)F)[C@@H](C)[C@H]1c1ccc(F)c(F)c1OCCN1CC2(COC2)C1]

4-((2R,3S,4S,5R)-3-(2-(2-(2-oxa-6-azaspiro[3.3]heptan-6-
yl)ethoxy)-3,4-difluorophenyl)-4,5-dimethyl-5-(trifluoro-
methyl)tetrahydrofuran-2-carboxamido)-5-methyl-
picolinamide

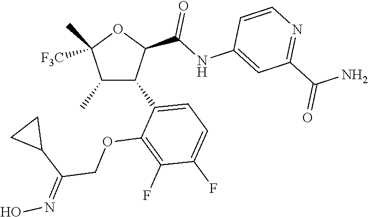 [SMILES: C[C@H]1[C@@H](c2ccc(F)c(F)c2OC/C(=N/O)C2CC2)[C@H](C(=O)Nc2ccnc(C(N)=O)c2)O[C@@]1(C)C(F)(F)F]

4-((2R,3S,4S,5R)-3-(2-((E)-2-cyclopropyl-2-(hydroxy-
imino)ethoxy)-3,4-difluorophenyl)-4,5-dimethyl-5-
(trifluoromethyl)tetrahydrofuran-2-carboxamido)-
picolinamide

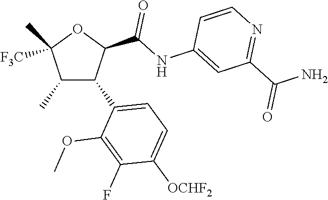 [SMILES: COc1c([C@H]2[C@H](C(=O)Nc3ccnc(C(N)=O)c3)O[C@@](C)(C(F)(F)F)[C@H]2C)ccc(OC(F)F)c1F]

4-((2R,3S,4S,5R)-3-(4-(difluoromethoxy)-3-fluoro-
2-methoxyphenyl)-4,5-dimethyl-5-(trifluorometh-
yl)tetrahydrofuran-2-carboxamido)picolinamide

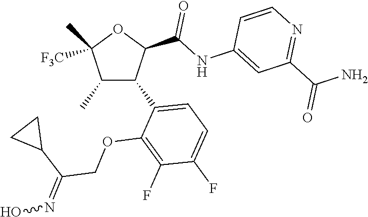 [SMILES: C[C@H]1[C@@H](c2ccc(F)c(F)c2OCC(=NO)C2CC2)[C@H](C(=O)Nc2ccnc(C(N)=O)c2)O[C@@]1(C)C(F)(F)F]

4-((2R,3S,4S,5R)-3-(2-(2-cyclopropyl-2-(hydroxyimino)-
ethoxy)-3,4-difluorophenyl)-4,5-dimethyl-5-(trifluoro-
methyl)tetrahydrofuran-2-carboxamido)picolinamide

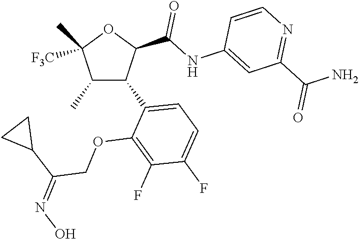 [SMILES: C[C@H]1[C@@H](c2ccc(F)c(F)c2OC/C(=N\O)C2CC2)[C@H](C(=O)Nc2ccnc(C(N)=O)c2)O[C@@]1(C)C(F)(F)F]

4-((2R,3S,4S,5R)-3-(2-((Z)-2-cyclopropyl-2-(hydroxy-
imino)ethoxy)-3,4-difluorophenyl)-4,5-dimethyl-5-
(trifluoromethyl)tetrahydrofuran-2-carboxamido)-
picolinamide

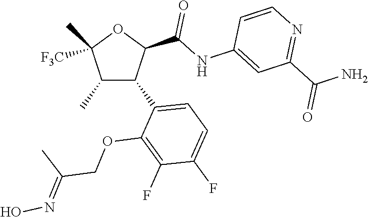 [SMILES: C/C(COc1c([C@H]2[C@H](C(=O)Nc3ccnc(C(N)=O)c3)O[C@@](C)(C(F)(F)F)[C@H]2C)ccc(F)c1F)=N\O]

4-((2R,3S,4S,5R)-3-(3,4-difluoro-2-((E)-2-(hydroxyimino)-
propoxy)phenyl)-4,5-dimethyl-5-(trifluoromethyl)tetrahydro-
furan-2-carboxamido)picolinamide

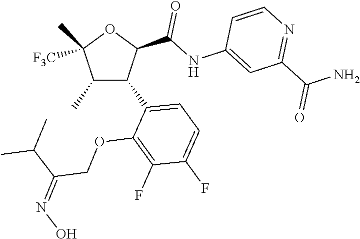 [SMILES: CC(C)/C(COc1c([C@H]2[C@H](C(=O)Nc3ccnc(C(N)=O)c3)O[C@@](C)(C(F)(F)F)[C@H]2C)ccc(F)c1F)=N/O]

4-((2R,3S,4S,5R)-3-(3,4-difluoro-2-((Z)-2-(hydroxyimino)-
3-methylbutoxy)phenyl)-4,5-dimethyl-5-(trifluoromethyl)-
tetrahydrofuran-2-carboxamido)picolinamide

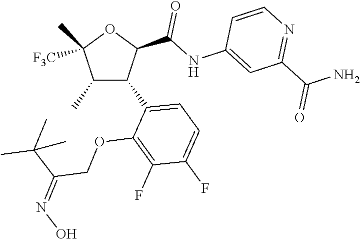 [SMILES: C[C@H]1[C@@H](c2ccc(F)c(F)c2OC/C(=N\O)C(C)(C)C)[C@H](C(=O)Nc2ccnc(C(N)=O)c2)O[C@@]1(C)C(F)(F)F]

4-((2R,3S,4S,5R)-3-(3,4-difluoro-2-((Z)-2-(hydroxyimino)-
3,3-dimethylbutoxy)phenyl)-4,5-dimethyl-5-(trifluoro-
methyl)tetrahydrofuran-2-carboxamido)picolinamide

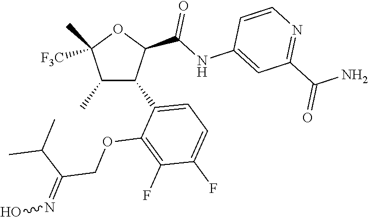 [SMILES: CC(C)C(COc1c([C@H]2[C@H](C(=O)Nc3ccnc(C(N)=O)c3)O[C@@](C)(C(F)(F)F)[C@H]2C)ccc(F)c1F)=NO]

4-((2R,3S,4S,5R)-3-(3,4-difluoro-2-(2-(hydroxyimino)-3-
methylbutoxy)phenyl)-4,5-dimethyl-5-(trifluoromethyl)-
tetrahydrofuran-2-carboxamido)picolinamide

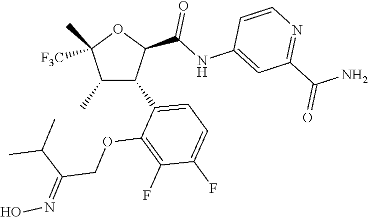 [SMILES: CC(C)/C(COc1c([C@H]2[C@H](C(=O)Nc3ccnc(C(N)=O)c3)O[C@@](C)(C(F)(F)F)[C@H]2C)ccc(F)c1F)=N\O]

4-((2R,3S,4S,5R)-3-(3,4-difluoro-2-((E)-2-(hydroxyimino)-
3-methylbutoxy)phenyl)-4,5-dimethyl-5-(trifluoromethyl)-
tetrahydrofuran-2-carboxamido)picolinamide

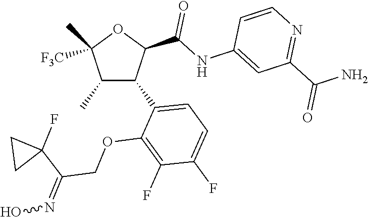 [SMILES: C[C@H]1[C@@H](c2ccc(F)c(F)c2OCC(=NO)C2(F)CC2)[C@H](C(=O)Nc2ccnc(C(N)=O)c2)O[C@@]1(C)C(F)(F)F]

4-((2R,3S,4S,5R)-3-(3,4-difluoro-2-(2-(1-fluorocyclopropyl)-
2-(hydroxyimino)ethoxy)phenyl)-4,5-dimethyl-5-(trifluoro-
methyl)tetrahydrofuran-2-carboxamido)picolinamide

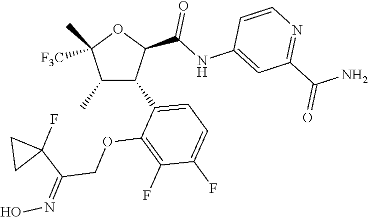 [SMILES: C[C@H]1[C@@H](c2ccc(F)c(F)c2OC/C(=N/O)C2(F)CC2)[C@H](C(=O)Nc2ccnc(C(N)=O)c2)O[C@@]1(C)C(F)(F)F]

4-((2R,3S,4S,5R)-3-(3,4-difluoro-2-((Z)-2-(1-fluorocyclo-
propyl)-2-(hydroxyimino)ethoxy)phenyl)-4,5-dimethyl-
5-(trifluoromethyl)tetrahydrofuran-2-carboxamido)-
picolinamide

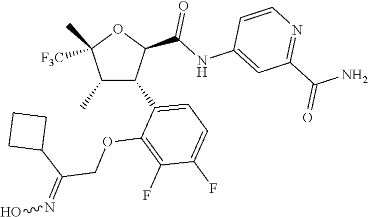 [SMILES: C[C@H]1[C@@H](c2ccc(F)c(F)c2OCC(=NO)C2CCC2)[C@H](C(=O)Nc2ccnc(C(N)=O)c2)O[C@@]1(C)C(F)(F)F]

4-((2R,3S,4S,5R)-3-(2-(2-cyclobutyl-2-(hydroxyimino)
ethoxy)-3,4-difluorophenyl)-4,5-dimethyl-5-(trifluoro-
methyl)tetrahydrofuran-2-carboxamido)picolinamide

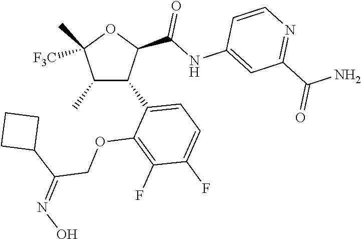 [SMILES: C[C@H]1[C@@H](c2ccc(F)c(F)c2OC/C(=N\O)C2CCC2)[C@H](C(=O)Nc2ccnc(C(N)=O)c2)O[C@@]1(C)C(F)(F)F]

4-((2R,3S,4S,5R)-3-(2-((Z)-2-cyclobutyl-2-(hydroxy-
imino)ethoxy)-3,4-difluorophenyl)-4,5-dimethyl-5-
(trifluoromethyl)tetrahydrofuran-2-carboxamido)-
picolinamide

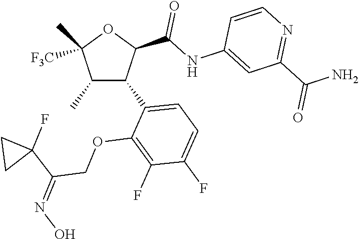 [SMILES: C[C@H]1[C@@H](c2ccc(F)c(F)c2OC/C(=N\O)C2(F)CC2)[C@H](C(=O)Nc2ccnc(C(N)=O)c2)O[C@@]1(C)C(F)(F)F]

4-((2R,3S,4S,5R)-3-(3,4-difluoro-2-((E)-2-(1-fluorocyclo-
propyl)-2-(hydroxyimino)ethoxy)phenyl)-4,5-dimethyl-
5-(trifluoromethyl)tetrahydrofuran-2-carboxamido)-
picolinamide

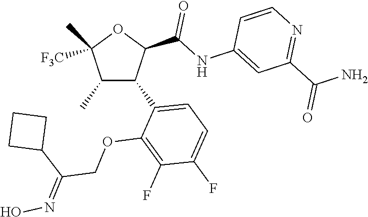 [SMILES: C[C@H]1[C@@H](c2ccc(F)c(F)c2OC/C(=N/O)C2CCC2)[C@H](C(=O)Nc2ccnc(C(N)=O)c2)O[C@@]1(C)C(F)(F)F]

4-((2R,3S,4S,5R)-3-(2-((E)-2-cyclobutyl-2-(hydroxyimino)-
ethoxy)-3,4-difluorophenyl)-4,5-dimethyl-5-(trifluorometh-
yl)tetrahydrofuran-2-carboxamido)picolinamide

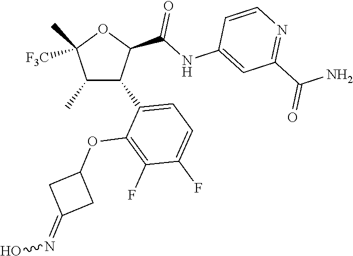 [SMILES: C[C@H]1[C@@H](c2ccc(F)c(F)c2OC2CC(=NO)C2)[C@H](C(=O)Nc2ccnc(C(N)=O)c2)O[C@@]1(C)C(F)(F)F]

4-((2R,3S,4S,5R)-3-(3,4-difluoro-2-(3-(hydroxyimino)-
cyclobutoxy)phenyl)-4,5-dimethyl-5-(trifluoromethyl)-
tetrahydrofuran-2-carboxamido)picolinamide

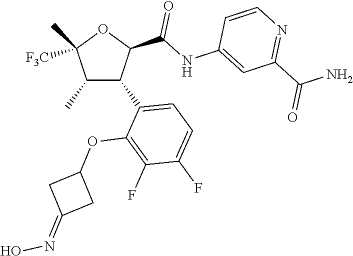 [SMILES: C[C@H]1[C@@H](c2ccc(F)c(F)c2OC2CC(=NO)C2)[C@H](C(=O)Nc2ccnc(C(N)=O)c2)O[C@@]1(C)C(F)(F)F]

4-((2R,3S,4S,5R)-3-(3,4-difluoro-2-(3-(hydroxyimino)-
cyclobutoxy)phenyl)-4,5-dimethyl-5-(trifluoromethyl)-
tetrahydrofuran-2-carboxamido)picolinamide

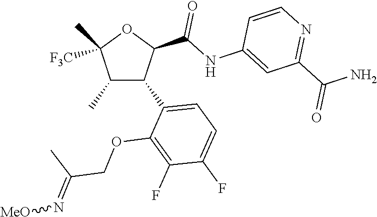 [SMILES: CON=C(C)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-(2-(methoxyimino)-
propoxy)phenyl)-4,5-dimethyl-5-(trifluoromethyl)tetra-
hydrofuran-2-carboxamido)picolinamide

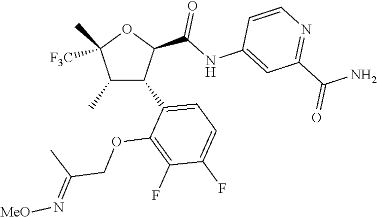 [SMILES: CO/N=C(\C)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((E)-2-(methoxyimino)-
propoxy)phenyl)-4,5-dimethyl-5-(trifluoromethyl)tetrahydro-
furan-2-carboxamido)picolinamide

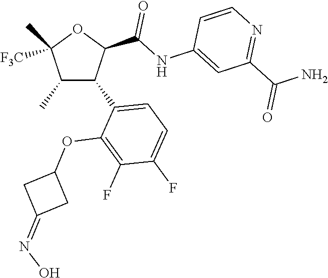 [SMILES: C[C@H]1[C@@H](c2ccc(F)c(F)c2OC2CC(=NO)C2)[C@H](C(=O)Nc2ccnc(C(N)=O)c2)O[C@@]1(C)C(F)(F)F]

4-((2R,3S,4S,5R)-3-(3,4-difluoro-2-(3-(hydroxy-
imino)cyclobutoxy)phenyl)-4,5-dimethyl-5-
(trifluoromethyl)tetrahydrofuran-2-carbox-
amido)picolinamide

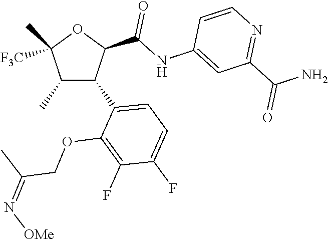 [SMILES: CO/N=C(/C)COc1c([C@H]2[C@H](C(=O)Nc3ccnc(C(N)=O)c3)O[C@@](C)(C(F)(F)F)[C@H]2C)ccc(F)c1F]

4-((2R,3S,4S,5R)-3-(3,4-difluoro-2-((Z)-2-(meth-
oxyimino)propoxy)phenyl)-4,5-dimethyl-5-
(trifluoromethyl)tetrahydrofuran-2-carboxamido)-
picolinamide

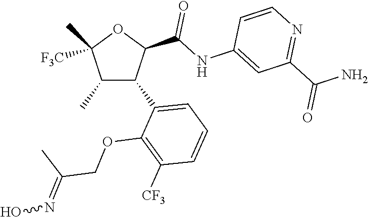 [SMILES: CC(COc1c([C@H]2[C@H](C(=O)Nc3ccnc(C(N)=O)c3)O[C@@](C)(C(F)(F)F)[C@H]2C)cccc1C(F)(F)F)=NO]

4-((2R,3S,4S,5R)-3-(2-(2-(hydroxyimino)propoxy)-3-
(trifluoromethyl)phenyl)-4,5-dimethyl-5-(trifluoro-
methyl)tetrahydrofuran-2-carboxamido)picolinamide

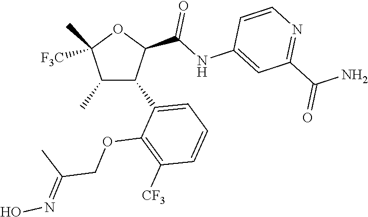 [SMILES: C/C(COc1c([C@H]2[C@H](C(=O)Nc3ccnc(C(N)=O)c3)O[C@@](C)(C(F)(F)F)[C@H]2C)cccc1C(F)(F)F)=N\O]

4-((2R,3S,4S,5R)-3-(2-((E)-2-(hydroxyimino)propoxy)-3-
(trifluoromethyl)phenyl)-4,5-dimethyl-5-(trifluoromethyl)-
tetrahydrofuran-2-carboxamido)picolinamide

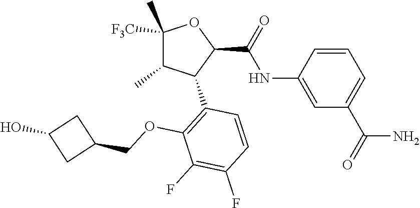 [SMILES: C[C@H]1[C@@H](c2ccc(F)c(F)c2OC[C@H]2C[C@H](O)C2)[C@H](C(=O)Nc2cccc(C(N)=O)c2)O[C@@]1(C)C(F)(F)F]

(2R,3S,4S,5R)-N-(3-carbamoylphenyl)-3-(3,4-difluoro-2-(((1r,3S)-
3-hydroxycyclobutyl)methoxy)phenyl)-4,5-dimethyl-5-(trifluoro-
methyl)tetrahydrofuran-2-carboxamide

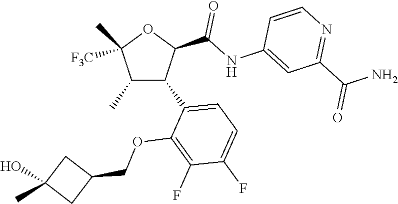 [SMILES: C[C@H]1[C@@H](c2ccc(F)c(F)c2OC[C@H]2C[C@](C)(O)C2)[C@H](C(=O)Nc2ccnc(C(N)=O)c2)O[C@@]1(C)C(F)(F)F]

4-((2R,3S,4S,5R)-3-(3,4-difluoro-2-(((1r,3S)-3-hydroxy-3-
methylcyclobutyl)methoxy)phenyl)-4,5-dimethyl-5-(trifluoro-
methyl)tetrahydrofuran-2-carboxamido)picolinamide

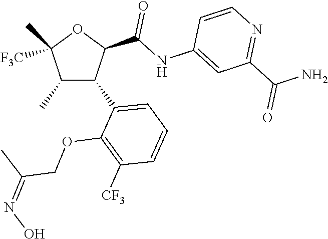 [SMILES: C/C(COc1c([C@H]2[C@H](C(=O)Nc3ccnc(C(N)=O)c3)O[C@@](C)(C(F)(F)F)[C@H]2C)cccc1C(F)(F)F)=N/O]

4-((2R,3S,4S,5R)-3-(2-((Z)-2-(hydroxyimino)
propoxy)-3-(trifluoromethyl)phenyl)-4,5-
dimethyl-5-(trifluoromethyl)tetrahydrofuran-
2-carboxamido)picolinamide

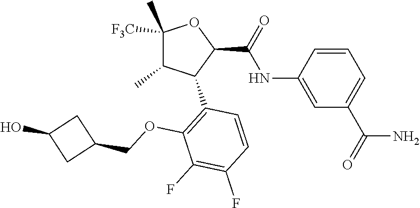 [SMILES: C[C@H]1[C@@H](c2ccc(F)c(F)c2OC[C@H]2C[C@@H](O)C2)[C@H](C(=O)Nc2cccc(C(N)=O)c2)O[C@@]1(C)C(F)(F)F]

(2R,3S,4S,5R)-N-(3-carbamoylphenyl)-3-(3,4-difluoro-2-(((1s,3R)-
3-hydroxycyclobutyl)methoxy)phenyl)-4,5-dimethyl-5-(trifluoro-
methyl)tetrahydrofuran-2-carboxamide

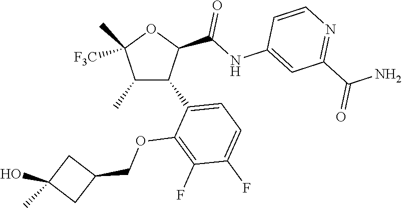 [SMILES: C[C@H]1[C@@H](c2ccc(F)c(F)c2OC[C@H]2C[C@@](C)(O)C2)[C@H](C(=O)Nc2ccnc(C(N)=O)c2)O[C@@]1(C)C(F)(F)F]

4-((2R,3S,4S,5R)-3-(3,4-difluoro-2-(((1s,3R)-3-hydroxy-3-meth-
ylcyclobutyl)methoxy)phenyl)-4,5-dimethyl-5-(trifluoromethyl)-
tetrahydrofuran-2-carboxamido)picolinamide

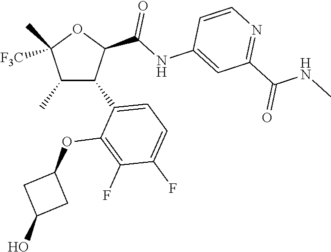 [SMILES: CNC(=O)c1cc(NC(=O)[C@@H]2O[C@@](C)(C(F)(F)F)[C@@H](C)[C@H]2c2ccc(F)c(F)c2O[C@H]2C[C@@H](O)C2)ccn1]

4-((2R,3S,4S,5R)-3-(3,4-difluoro-2-((1s,3R)-3-
hydroxycyclobutoxy)phenyl)-4,5-dimethyl-5-
(trifluoromethyl)tetrahydrofuran-2-carboxamido)-
N-methylpicolinamide

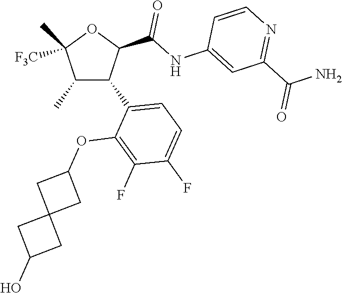 [SMILES: C[C@H]1[C@@H](c2ccc(F)c(F)c2OC2CC3(CC(O)C3)C2)[C@H](C(=O)Nc2ccnc(C(N)=O)c2)O[C@@]1(C)C(F)(F)F]

4-((2R,3S,4S,5R)-3-(3,4-difluoro-2-((6-hydroxyspiro
[3.3]heptan-2-yl)oxy)phenyl)-4,5-dimethyl-5-(trifluoro-
methyl)tetrahydrofuran-2-carboxamido)picolinamide

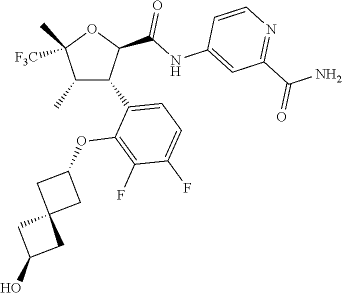

4-((2R,3S,4S,5R)-3-(3,4-difluoro-2-(((2S,4r,6S)-6-
hydroxyspiro[3.3]heptan-2-yl)oxy)phenyl)-4,5-
dimethyl-5-(trifluoromethyl)tetrahydrofuran-2-
carboxamido)picolinamide

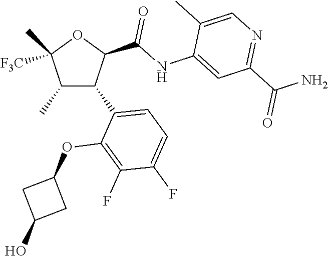 [SMILES: Cc1cnc(C(N)=O)cc1NC(=O)[C@@H]1O[C@@](C)(C(F)(F)F)[C@@H](C)[C@H]1c1ccc(F)c(F)c1O[C@H]1C[C@@H](O)C1]

4-((2R,3S,4S,5R)-3-(3,4-difluoro-2-((1s,3R)-3-
hydroxycyclobutoxy)phenyl)-4,5-dimethyl-5-
(trifluoromethyl)tetrahydrofuran-2-carboxamido)-
5-methylpicolinamide

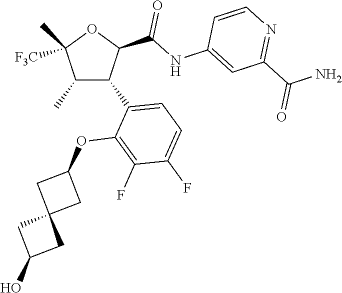

4-((2R,3S,4S,5R)-3-(3,4-difluoro-2-(((2R,4s,6R)-6-
hydroxyspiro[3.3]heptan-2-yl)oxy)phenyl)-4,5-
dimethyl-5-(trifluoromethyl)tetrahydrofuran-2-
carboxamido)picolinamide

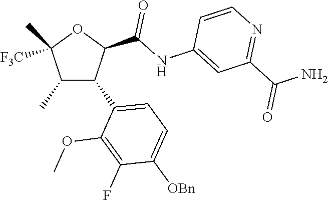 [SMILES: COc1c([C@H]2[C@H](C(=O)Nc3ccnc(C(N)=O)c3)O[C@@](C)(C(F)(F)F)[C@H]2C)ccc(OCc2ccccc2)c1F]

4-((2R,3S,4S,5R)-3-(4-(benzyloxy)-3-fluoro-2-
methoxyphenyl)-4,5-dimethyl-5-(trifluoromethyl)-
tetrahydrofuran-2-carboxamido)picolinamide

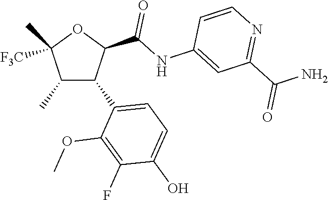 [SMILES: COc1c([C@H]2[C@H](C(=O)Nc3ccnc(C(N)=O)c3)O[C@@](C)(C(F)(F)F)[C@H]2C)ccc(O)c1F]

4-((2R,3S,4S,5R)-3-(3-fluoro-4-hydroxy-2-methoxy-
phenyl)-4,5-dimethyl-5-(trifluoromethyl)tetrahydro-
furan-2-carboxamido)picolinamide

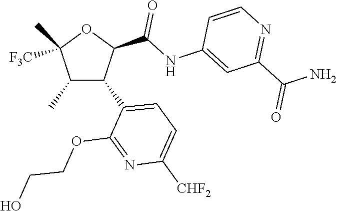 [SMILES: C[C@H]1[C@@H](c2ccc(C(F)F)nc2OCCO)[C@H](C(=O)Nc2ccnc(C(N)=O)c2)O[C@@]1(C)C(F)(F)F]

4-((2R,3S,4S,5R)-3-(6-(difluoromethyl)-2-(2-hydroxy-
ethoxy)pyridin-3-yl)-4,5-dimethyl-5-(trifluoromethyl)-
tetrahydrofuran-2-carboxamido)picolinamide

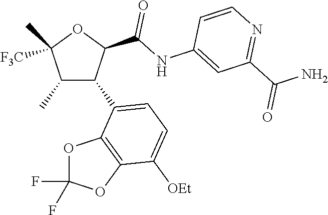 [SMILES: CCOc1ccc([C@H]2[C@H](C(=O)Nc3ccnc(C(N)=O)c3)O[C@@](C)(C(F)(F)F)[C@H]2C)c2c1OC(F)(F)O2]

4-((2R,3S,4S,5R)-3-(7-ethoxy-2,2-difluorobenzo[d]
[1,3]dioxol-4-yl)-4,5-dimethyl-5-(trifluoromethyl)-
tetrahydrofuran-2-carboxamido)picolinamide

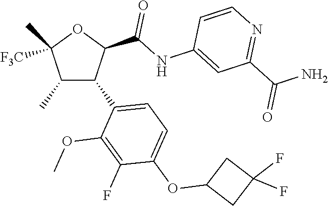 [SMILES: COc1c([C@H]2[C@H](C(=O)Nc3ccnc(C(N)=O)c3)O[C@@](C)(C(F)(F)F)[C@H]2C)ccc(OC2CC(F)(F)C2)c1F]

4-((2R,3S,4S,5R)-3-(4-(3,3-difluorocyclobutoxy)-3-
fluoro-2-methoxyphenyl)-4,5-dimethyl-5-(trifluoro-
methyl)tetrahydrofuran-2-carboxamido)picolinamide

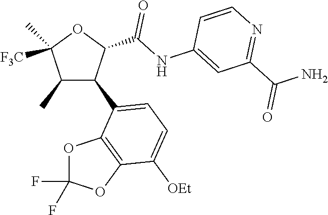 [SMILES: CCOc1ccc([C@@H]2[C@@H](C(=O)Nc3ccnc(C(N)=O)c3)O[C@](C)(C(F)(F)F)[C@@H]2C)c2c1OC(F)(F)O2]

4-((2S,3R,4R,5S)-3-(7-ethoxy-2,2-difluorobenzo[d]
[1,3]dioxol-4-yl)-4,5-dimethyl-5-(trifluoromethyl)-
tetrahydrofuran-2-carboxamido)picolinamide

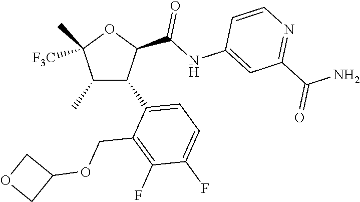 [SMILES: C[C@H]1[C@@H](c2ccc(F)c(F)c2COC2COC2)[C@H](C(=O)Nc2ccnc(C(N)=O)c2)O[C@@]1(C)C(F)(F)F]

4-((2R,3S,4S,5R)-3-(3,4-difluoro-2-((oxetan-3-yloxy)-
methyl)phenyl)-4,5-dimethyl-5-(trifluoromethyl)tetra-
hydrofuran-2-carboxamido)picolinamide

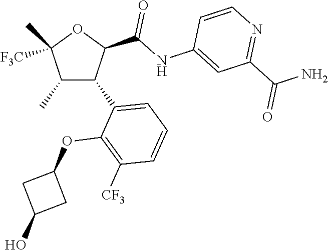 [SMILES: C[C@H]1[C@@H](c2cccc(C(F)(F)F)c2O[C@H]2C[C@@H](O)C2)[C@H](C(=O)Nc2ccnc(C(N)=O)c2)O[C@@]1(C)C(F)(F)F]

4-((2R,3S,4S,5R)-3-(2-((1s,3R)-3-hydroxycyclo-
butoxy)-3-(trifluoromethyl)phenyl)-4,5-dimethyl-5-
(trifluoromethyl)tetrahydrofuran-2-carboxamido)-
picolinamide

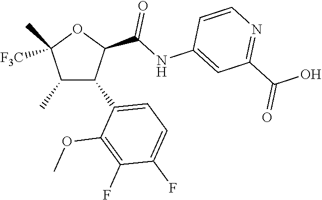 [SMILES: COc1c([C@H]2[C@H](C(=O)Nc3ccnc(C(=O)O)c3)O[C@@](C)(C(F)(F)F)[C@H]2C)ccc(F)c1F]

4-((2R,3S,4S,5R)-3-(3,4-difluoro-2-methoxyphen-
yl)-4,5-dimethyl-5-(trifluoromethyl)tetrahydrofuran-
2-carboxamido)picolinic acid

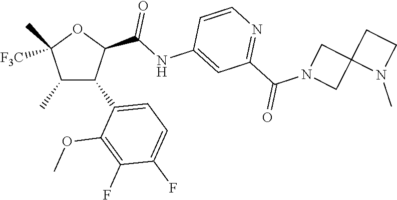 [SMILES: COc1c([C@H]2[C@H](C(=O)Nc3ccnc(C(=O)N4CC5(CCN5C)C4)c3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(2-(1-methyl-1,6-diazaspiro[3.3]heptane-6-carbonyl)pyridin-
4-yl)-5-(trifluoromethyl)tetrahydrofuran-2-carboxamide

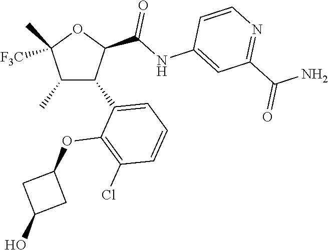 [SMILES: C[C@H]1[C@@H](c2cccc(Cl)c2O[C@H]2C[C@@H](O)C2)[C@H](C(=O)Nc2ccnc(C(N)=O)c2)O[C@@]1(C)C(F)(F)F]

4-((2R,3S,4S,5R)-3-(3-chloro-2-((1s,3R)-3-hydroxy-
cyclobutoxy)phenyl)-4,5-dimethyl-5-(trifluorometh-
yl)tetrahydrofuran-2-carboxamido)picolinamide

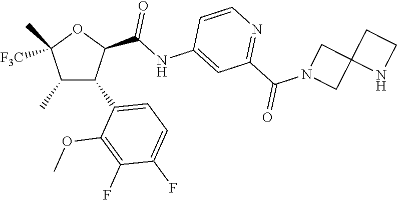 [SMILES: COc1c([C@H]2[C@H](C(=O)Nc3ccnc(C(=O)N4CC5(CCN5)C4)c3)O[C@@](C)(C(F)(F)F)[C@H]2C)ccc(F)c1F]

(2R,3S,4S,5R)-N-(2-(1,6-diazaspiro[3.3]heptane-6-carbonyl)-
pyridin-4-yl)-3-(3,4-difluoro-2-methoxyphenyl)-4,5-dimethyl-
5-(trifluoromethyl)tetrahydrofuran-2-carboxamide

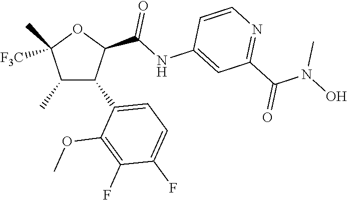 [SMILES: COc1c([C@H]2[C@H](C(=O)Nc3ccnc(C(=O)N(C)O)c3)O[C@@](C)(C(F)(F)F)[C@H]2C)ccc(F)c1F]

4-((2R,3S,4S,5R)-3-(3,4-difluoro-2-methoxyphenyl)-
4,5-dimethyl-5-(trifluoromethyl)tetrahydrofuran-2-
carboxamido)-N-hydroxy-N-methylpicolinamide

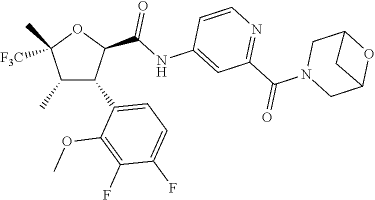 [SMILES: COc1c([C@H]2[C@H](C(=O)Nc3ccnc(C(=O)N4CC5CC(C4)O5)c3)O[C@@](C)(C(F)(F)F)[C@H]2C)ccc(F)c1F]

(2R,3S,4S,5R)-N-(2-(6-oxa-3-azabicyclo[3.1.1]heptane-3-
carbonyl)pyridin-4-yl)-3-(3,4-difluoro-2-methoxyphenyl)-4,5-
dimethyl-5-(trifluoromethyl)tetrahydrofuran-2-carboxamide

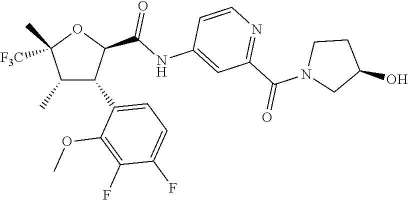 [SMILES: COc1c([C@H]2[C@H](C(=O)Nc3ccnc(C(=O)N4CC[C@@H](O)C4)c3)O[C@@](C)(C(F)(F)F)[C@H]2C)ccc(F)c1F]

(2R,3S,4S,5R)-3-(3,4-difluoro-2-methoxyphenyl)-N-(2-((R)-3-
hydroxypyrrolidine-1-carbonyl)pyridin-4-yl)-4,5-dimethyl-5-
(trifluoromethyl)tetrahydrofuran-2-carboxamide

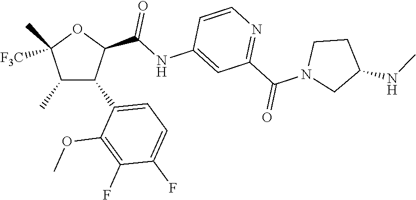 [SMILES: CN[C@H]1CCN(C(=O)c2cc(NC(=O)[C@@H]3O[C@@](C)(C(F)(F)F)[C@@H](C)[C@H]3c3ccc(F)c(F)c3OC)ccn2)C1]

(2R,3S,4S,5R)-3-(3,4-difluoro-2-methoxyphenyl)-4,5-dimethyl-
N-(2-((S)-3-(methylamino)pyrrolidine-1-carbonyl)pyridin-4-yl)-
5-(trifluoromethyl)tetrahydrofuran-2-carboxamide

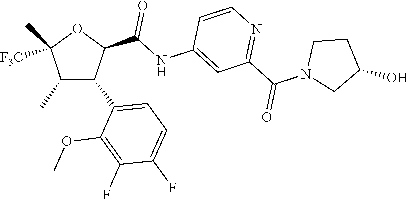 [SMILES: COc1c([C@H]2[C@H](C(=O)Nc3ccnc(C(=O)N4CC[C@H](O)C4)c3)O[C@@](C)(C(F)(F)F)[C@H]2C)ccc(F)c1F]

(2R,3S,4S,5R)-3-(3,4-difluoro-2-methoxyphenyl)-N-(2-((S)-3-
hydroxypyrrolidine-1-carbonyl)pyridin-4-yl)-4,5-dimethyl-5-
(trifluoromethyl)tetrahydrofuran-2-carboxamide

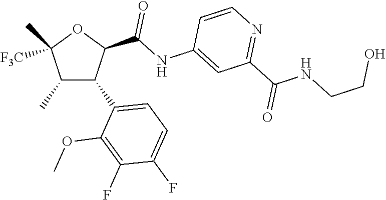 [SMILES: COc1c([C@H]2[C@H](C(=O)Nc3ccnc(C(=O)NCCO)c3)O[C@@](C)(C(F)(F)F)[C@H]2C)ccc(F)c1F]

4-((2R,3S,4S,5R)-3-(3,4-difluoro-2-methoxyphenyl)-4,5-
dimethyl-5-(trifluoromethyl)tetrahydrofuran-2-carboxamido)-
N-(2-hydroxyethyl)picolinamide

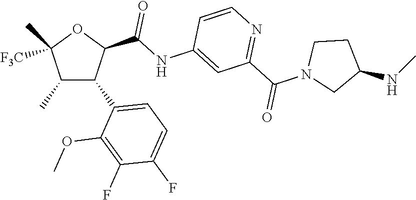 [SMILES: CN[C@@H]1CCN(C(=O)c2cc(NC(=O)[C@@H]3O[C@@](C)(C(F)(F)F)[C@@H](C)[C@H]3c3ccc(F)c(F)c3OC)ccn2)C1]

(2R,3S,4S,5R)-3-(3,4-difluoro-2-methoxyphenyl)-4,5-dimethyl-N-
(2-((R)-3-(methylamino)pyrrolidine-1-carbonyl)pyridin-4-yl)-5-
(trifluoromethyl)tetrahydrofuran-2-carboxamide

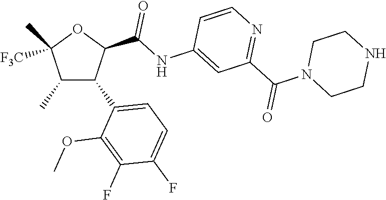 [SMILES: COc1c([C@H]2[C@H](C(=O)Nc3ccnc(C(=O)N4CCNCC4)c3)O[C@@](C)(C(F)(F)F)[C@H]2C)ccc(F)c1F]

(2R,3S,4S,5R)-3-(3,4-difluoro-2-methoxyphenyl)-4,5-dimeth-
yl-N-(2-(piperazine-1-carbonyl)pyridin-4-yl)-5-(trifluorometh-
yl)tetrahydrofuran-2-carboxamide

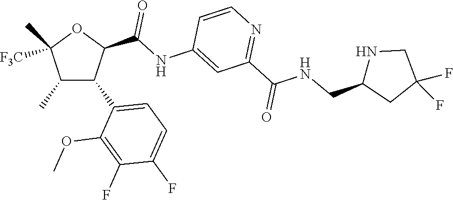 [SMILES: COc1c([C@H]2[C@H](C(=O)Nc3ccnc(C(=O)NC[C@@H]4CC(F)(F)CN4)c3)O[C@@](C)(C(F)(F)F)[C@H]2C)ccc(F)c1F]

4-((2R,3S,4S,5R)-3-(3,4-difluoro-2-methoxyphenyl)-4,5-dimethyl-5-
(trifluoromethyl)tetrahydrofuran-2-carboxamido)-N-(((S)-4,4-difluoro
pyrrolidin-2-yl)methyl)picolinamide

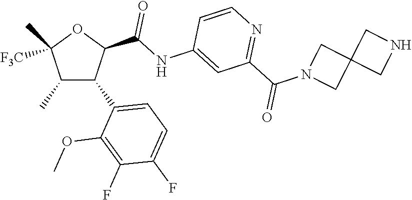 [SMILES: COc1c([C@H]2[C@H](C(=O)Nc3ccnc(C(=O)N4CC5(CNC5)C4)c3)O[C@@](C)(C(F)(F)F)[C@H]2C)ccc(F)c1F]

(2R,3S,4S,5R)-N-(2-(2,6-diazaspiro[3.3]heptane-2-carbonyl)-
pyridin-4-yl)-3-(3,4-difluoro-2-methoxyphenyl)-4,5-dimethyl-
5-(trifluoromethyl)tetrahydrofuran-2-carboxamide

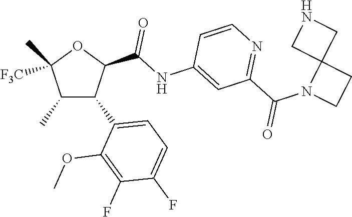 [SMILES: COc1c([C@H]2[C@H](C(=O)Nc3ccnc(C(=O)N4CCC45CNC5)c3)O[C@@](C)(C(F)(F)F)[C@H]2C)ccc(F)c1F]

(2R,3S,4S,5R)-N-(2-(1,6-diazaspiro[3.3]heptane-1-
carbonyl)pyridin-4-yl)-3-(3,4-difluoro-2-methoxy-
phenyl)-4,5-dimethyl-5-(trifluoromethyl)tetrahydro-
furan-2-carboxamide

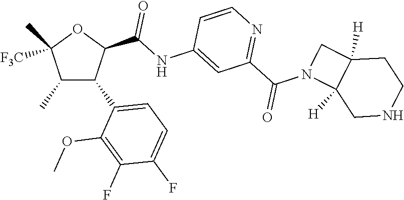 [SMILES: COc1c([C@H]2[C@H](C(=O)Nc3ccnc(C(=O)N4C[C@H]5CCNC[C@H]54)c3)O[C@@](C)(C(F)(F)F)[C@H]2C)ccc(F)c1F]

(2R,3S,4S,5R)-N-(2-((1S,6R)-3,8-diazabicyclo[4.2.0]octane-8-
carbonyl)pyridin-4-yl)-3-(3,4-difluoro-2-methoxyphenyl)-4,5-
dimethyl-5-(trifluoromethyl)tetrahydrofuran-2-carboxamide

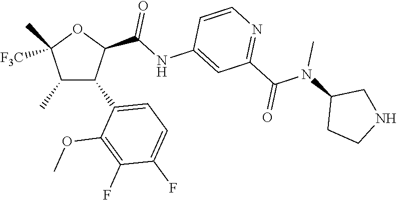 [SMILES: COc1c([C@H]2[C@H](C(=O)Nc3ccnc(C(=O)N(C)[C@@H]4CCNC4)c3)O[C@@](C)(C(F)(F)F)[C@H]2C)ccc(F)c1F]

4-((2R,3S,4S,5R)-3-(3,4-difluoro-2-methoxyphenyl)-4,5-
dimethyl-5-(trifluoromethyl)tetrahydrofuran-2-carboxamido)-
N-methyl-N-((R)-pyrrolidin-3-yl)picolinamide

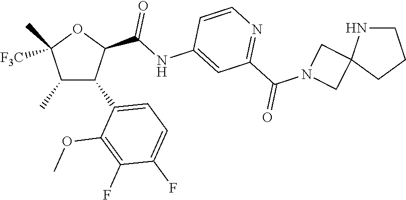 [SMILES: COc1c([C@H]2[C@H](C(=O)Nc3ccnc(C(=O)N4CC5(CCCN5)C4)c3)O[C@@](C)(C(F)(F)F)[C@H]2C)ccc(F)c1F]

(2R,3S,4S,5R)-N-(2-(2,5-diazaspiro[3.4]octane-2-carbonyl)-
pyridin-4-yl)-3-(3,4-difluoro-2-methoxyphenyl)-4,5-dimethyl-
5-(trifluoromethyl)tetrahydrofuran-2-carboxamide

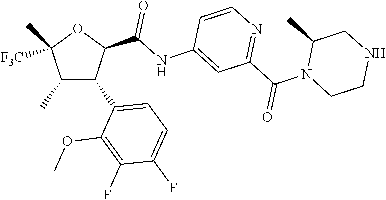 [SMILES: COc1c([C@H]2[C@H](C(=O)Nc3ccnc(C(=O)N4CCNC[C@@H]4C)c3)O[C@@](C)(C(F)(F)F)[C@H]2C)ccc(F)c1F]

(2R,3S,4S,5R)-3-(3,4-difluoro-2-methoxyphenyl)-4,5-dimeth-
yl-N-(2-((S)-2-methylpiperazine-1-carbonyl)pyridin-4-yl)-5-
(trifluoromethyl)tetrahydrofuran-2-carboxamide

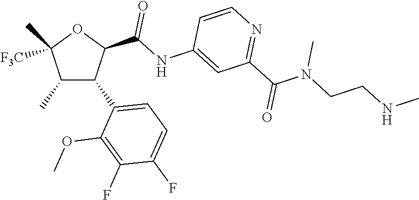 [SMILES: CNCCN(C)C(=O)c1cc(NC(=O)[C@@H]2O[C@@](C)(C(F)(F)F)[C@@H](C)[C@H]2c2ccc(F)c(F)c2OC)ccn1]

4-((2R,3S,4S,5R)-3-(3,4-difluoro-2-methoxyphenyl)-4,5-dimethyl-
5-(trifluoromethyl)tetrahydrofuran-2-carboxamido)-N-methyl-N-
(2-(methylamino)ethyl)picolinamide

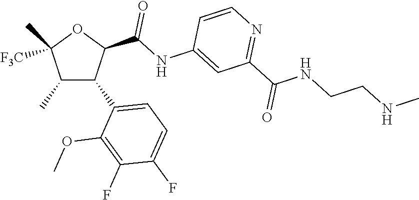 [SMILES: CNCCNC(=O)c1cc(NC(=O)[C@@H]2O[C@@](C)(C(F)(F)F)[C@@H](C)[C@H]2c2ccc(F)c(F)c2OC)ccn1]

4-((2R,3S,4S,5R)-3-(3,4-difluoro-2-methoxyphenyl)-4,5-dimethyl-
5-(trifluoromethyl)tetrahydrofuran-2-carboxamido)-N-(2-(methyl-
amino)ethyl)picolinamide

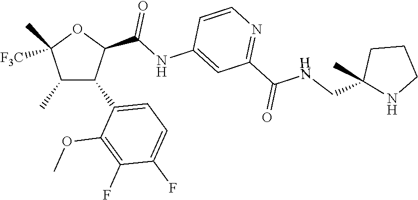 [SMILES: COc1c([C@H]2[C@H](C(=O)Nc3ccnc(C(=O)NC[C@]4(C)CCCN4)c3)O[C@@](C)(C(F)(F)F)[C@H]2C)ccc(F)c1F]

4-((2R,3S,4S,5R)-3-(3,4-difluoro-2-methoxyphenyl)-4,5-dimethyl-
5-(trifluoromethyl)tetrahydrofuran-2-carboxamido)-N-(((S)-2-
methylpyrrolidin-2-yl)methyl)picolinamide

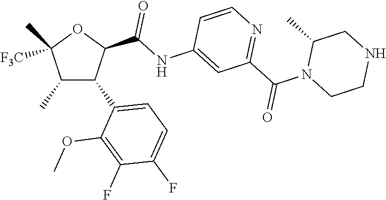 [SMILES: COc1c([C@H]2[C@H](C(=O)Nc3ccnc(C(=O)N4CCNC[C@H]4C)c3)O[C@@](C)(C(F)(F)F)[C@H]2C)ccc(F)c1F]

(2R,3S,4S,5R)-3-(3,4-difluoro-2-methoxyphenyl)-4,5-dimeth-
yl-N-(2-((R)-2-methylpiperazine-1-carbonyl)pyridin-4-yl)-5-
(trifluoromethyl)tetrahydrofuran-2-carboxamide

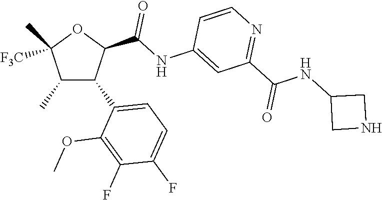 [SMILES: COc1c([C@H]2[C@H](C(=O)Nc3ccnc(C(=O)NC4CNC4)c3)O[C@@](C)(C(F)(F)F)[C@H]2C)ccc(F)c1F]

N-(azetidin-3-yl)-4-((2R,3S,4S,5R)-3-(3,4-difluoro-2-meth-
oxyphenyl)-4,5-dimethyl-5-(trifluoromethyl)tetrahydrofuran-
2-carboxamido)picolinamide

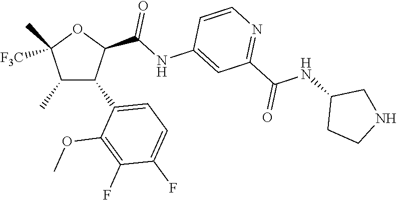 [SMILES: COc1c([C@H]2[C@H](C(=O)Nc3ccnc(C(=O)N[C@H]4CCNC4)c3)O[C@@](C)(C(F)(F)F)[C@H]2C)ccc(F)c1F]

4-((2R,3S,4S,5R)-3-(3,4-difluoro-2-methoxyphenyl)-4,5-
dimethyl-5-(trifluoromethyl)tetrahydrofuran-2-carboxamido)-
N-((S)-pyrrolidin-3-yl)picolinamide

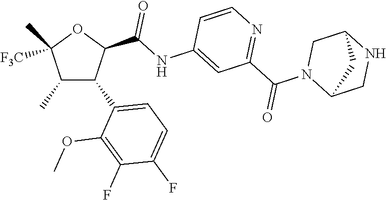 [SMILES: COc1c([C@H]2[C@H](C(=O)Nc3ccnc(C(=O)N4C[C@@H]5C[C@H]4CN5)c3)O[C@@](C)(C(F)(F)F)[C@H]2C)ccc(F)c1F]

(2R,3S,4S,5R)-N-(2-((1S,4S)-2,5-diazabicyclo[2.2.1]heptane-2-
carbonyl)pyridin-4-yl)-3-(3,4-difluoro-2-methoxyphenyl)-4,5-
dimethyl-5-(trifluoromethyl)tetrahydrofuran-2-carboxamide

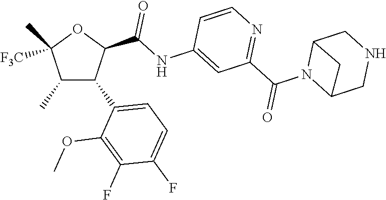 [SMILES: COc1c([C@H]2[C@H](C(=O)Nc3ccnc(C(=O)N4C5CNCC4C5)c3)O[C@@](C)(C(F)(F)F)[C@H]2C)ccc(F)c1F]

(2R,3S,4S,5R)-N-(2-(3,6-diazabicyclo[3.1.1]heptane-6-carbon-
yl)pyridin-4-yl)-3-(3,4-difluoro-2-methoxyphenyl)-4,5-dimeth-
yl-5-(trifluoromethyl)tetrahydrofuran-2-carboxamide

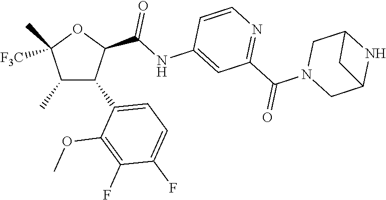 [SMILES: COc1c([C@H]2[C@H](C(=O)Nc3ccnc(C(=O)N4CC5CC(C4)N5)c3)O[C@@](C)(C(F)(F)F)[C@H]2C)ccc(F)c1F]

(2R,3S,4S,5R)-N-(2-(3,6-diazabicyclo[3.1.1]heptane-3-carbon-
yl)pyridin-4-yl)-3-(3,4-difluoro-2-methoxyphenyl)-4,5-dimeth-
yl-5-(trifluoromethyl)tetrahydrofuran-2-carboxamide

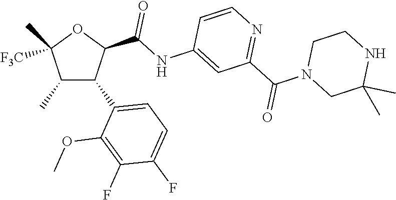 [SMILES: COc1c([C@H]2[C@H](C(=O)Nc3ccnc(C(=O)N4CCNC(C)(C)C4)c3)O[C@@](C)(C(F)(F)F)[C@H]2C)ccc(F)c1F]

(2R,3S,4S,5R)-3-(3,4-difluoro-2-methoxyphenyl)-N-(2-(3,3-
dimethylpiperazine-1-carbonyl)pyridin-4-yl)-4,5-dimethyl-5-
(trifluoromethyl)tetrahydrofuran-2-carboxamide

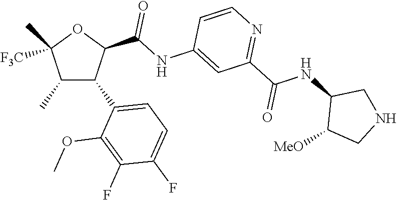 [SMILES: COc1c([C@H]2[C@H](C(=O)Nc3ccnc(C(=O)N[C@H]4CNC[C@@H]4OC)c3)O[C@@](C)(C(F)(F)F)[C@H]2C)ccc(F)c1F]

4-((2R,3S,4S,5R)-3-(3,4-difluoro-2-methoxyphenyl)-4,5-
dimethyl-5-(trifluoromethyl)tetrahydrofuran-2-carboxamido)-
N-((3S,4S)-4-methoxypyrrolidin-3-yl)picolinamide

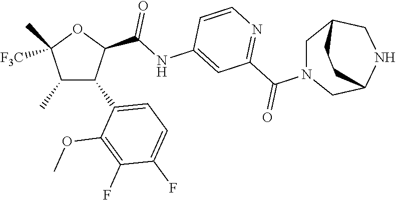 [SMILES: COc1c([C@H]2[C@H](C(=O)Nc3ccnc(C(=O)N4C[C@H]5CC[C@@H](C4)NC5)c3)O[C@@](C)(C(F)(F)F)[C@H]2C)ccc(F)c1F]

(2R,3S,4S,5R)-N-(2-((1S,5S)-3,6-diazabicyclo[3.2.2]nonane-3-
carbonyl)pyridin-4-yl)-3-(3,4-difluoro-2-methoxyphenyl)-4,5-
dimethyl-5-(trifluoromethyl)tetrahydrofuran-2-carboxamide

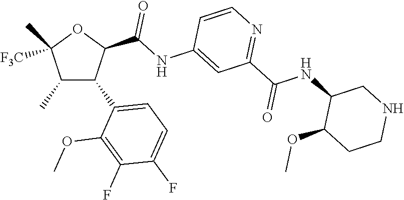 [SMILES: COc1c([C@H]2[C@H](C(=O)Nc3ccnc(C(=O)N[C@H]4CNCC[C@H]4OC)c3)O[C@@](C)(C(F)(F)F)[C@H]2C)ccc(F)c1F]

4-((2R,3S,4S,5R)-3-(3,4-difluoro-2-methoxyphenyl)-4,5-dimeth-
yl-5-(trifluoromethyl)tetrahydrofuran-2-carboxamido)-N-
((3S,4R)-4-methoxypiperidin-3-yl)picolinamide

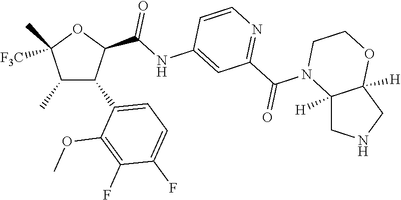 [SMILES: COc1c([C@H]2[C@H](C(=O)Nc3ccnc(C(=O)N4CCO[C@H]5CNC[C@H]54)c3)O[C@@](C)(C(F)(F)F)[C@H]2C)ccc(F)c1F]

(2R,3S,4S,5R)-3-(3,4-difluoro-2-methoxyphenyl)-4,5-dimethyl-N-
(2-((4aR,7aS)-octahydropyrrolo[3,4-b][1,4]oxazine-4-carbonyl)-
pyridin-4-yl)-5-(trifluoromethyl)tetrahydrofuran-2-carboxamide

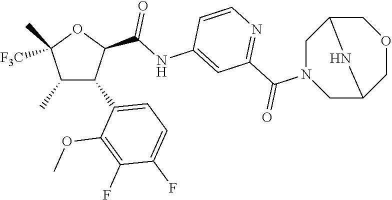 [SMILES: COc1c([C@H]2[C@H](C(=O)Nc3ccnc(C(=O)N4CC5COCC(C4)N5)c3)O[C@@](C)(C(F)(F)F)[C@H]2C)ccc(F)c1F]

(2R,3S,4S,5R)-N-(2-(3-oxa-7,9-diazabicyclo[3.3.1]nonane-7-
carbonyl)pyridin-4-yl)-3-(3,4-difluoro-2-methoxyphenyl)-4,5-
dimethyl-5-(trifluoromethyl)tetrahydrofuran-2-carboxamide

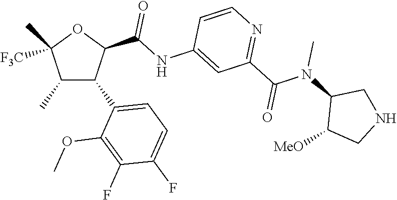 [SMILES: COc1c([C@H]2[C@H](C(=O)Nc3ccnc(C(=O)N(C)[C@H]4CNC[C@@H]4OC)c3)O[C@@](C)(C(F)(F)F)[C@H]2C)ccc(F)c1F]

4-((2R,3S,4S,5R)-3-(3,4-difluoro-2-methoxyphenyl)-4,5-dimeth-
yl-5-(trifluoromethyl)tetrahydrofuran-2-carboxamido)-N-
((3S,4S)-4-methoxypyrrolidin-3-yl)-N-methylpicolinamide

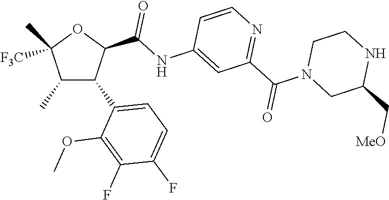 [SMILES: COC[C@H]1CN(C(=O)c2cc(NC(=O)[C@@H]3O[C@@](C)(C(F)(F)F)[C@@H](C)[C@H]3c3ccc(F)c(F)c3OC)ccn2)CCN1]

(2R,3S,4S,5R)-3-(3,4-difluoro-2-methoxyphenyl)-N-(2-((R)-3-
(methoxymethyl)piperazine-1-carbonyl)pyridin-4-yl)-4,5-
dimethyl-5-(trifluoromethyl)tetrahydrofuran-2-carboxamide

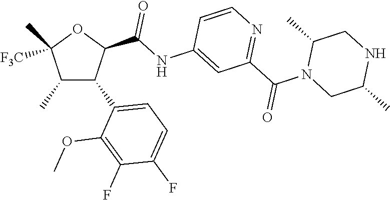 [SMILES: COc1c([C@H]2[C@H](C(=O)Nc3ccnc(C(=O)N4C[C@@H](C)NC[C@H]4C)c3)O[C@@](C)(C(F)(F)F)[C@H]2C)ccc(F)c1F]

(2R,3S,4S,5R)-3-(3,4-difluoro-2-methoxyphenyl)-N-(2-
((2R,5R)-2,5-dimethylpiperazine-1-carbonyl)pyridin-4-
yl)-4,5-dimethyl-5-(trifluoromethyl)tetrahydrofuran-2-
carboxamide

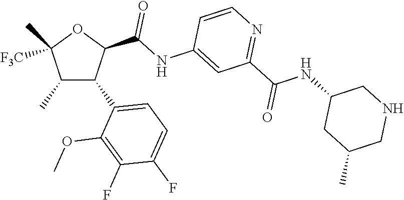 [SMILES: COc1c([C@H]2[C@H](C(=O)Nc3ccnc(C(=O)N[C@@H]4CNC[C@H](C)C4)c3)O[C@@](C)(C(F)(F)F)[C@H]2C)ccc(F)c1F]

4-((2R,3S,4S,5R)-3-(3,4-difluoro-2-methoxyphenyl)-4,5-dimeth-
yl-5-(trifluoromethyl)tetrahydrofuran-2-carboxamido)-N-
((3S,5R)-5-methylpiperidin-3-yl)picolinamide

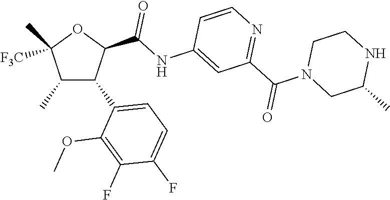 [SMILES: COc1c([C@H]2[C@H](C(=O)Nc3ccnc(C(=O)N4CCN[C@H](C)C4)c3)O[C@@](C)(C(F)(F)F)[C@H]2C)ccc(F)c1F]

(2R,3S,4S,5R)-3-(3,4-difluoro-2-methoxyphenyl)-4,5-dimeth-
yl-N-(2-((R)-3-methylpiperazine-1-carbonyl)pyridin-4-yl)-5-
(trifluoromethyl)tetrahydrofuran-2-carboxamide

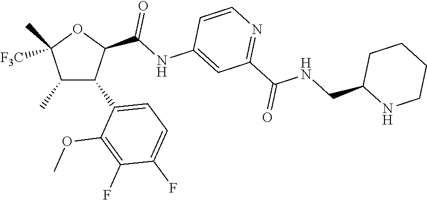 [SMILES: COc1c([C@H]2[C@H](C(=O)Nc3ccnc(C(=O)NC[C@H]4CCCCN4)c3)O[C@@](C)(C(F)(F)F)[C@H]2C)ccc(F)c1F]

4-((2R,3S,4S,5R)-3-(3,4-difluoro-2-methoxyphenyl)-4,5-dimethyl-5-
(trifluoromethyl)tetrahydrofuran-2-carboxamido)-N-(((R)-piperidin-
2-yl)methyl)picolinamide

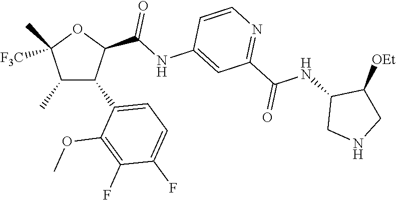 [SMILES: CCO[C@H]1CNC[C@@H]1NC(=O)c1cc(NC(=O)[C@@H]2O[C@@](C)(C(F)(F)F)[C@@H](C)[C@H]2c2ccc(F)c(F)c2OC)ccn1]

4-((2R,3S,4S,5R)-3-(3,4-difluoro-2-methoxyphenyl)-4,5-
dimethyl-5-(trifluoromethyl)tetrahydrofuran-2-carboxamido)-
N-((3S,4S)-4-ethoxypyrrolidin-3-yl)picolinamide

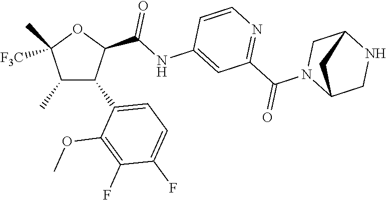 [SMILES: COc1c([C@H]2[C@H](C(=O)Nc3ccnc(C(=O)N4C[C@H]5C[C@@H]4CN5)c3)O[C@@](C)(C(F)(F)F)[C@H]2C)ccc(F)c1F]

(2R,3S,4S,5R)-N-(2-((1R,4R)-2,5-diazabicyclo[2.2.1]heptane-
2-carbonyl)pyridin-4-yl)-3-(3,4-difluoro-2-methoxyphenyl)-4,5-
dimethyl-5-(trifluoromethyl)tetrahydrofuran-2-carboxamide

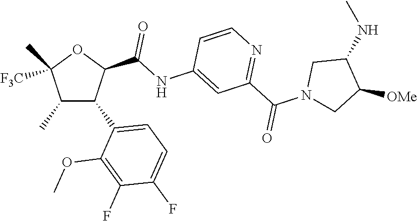 [SMILES: CN[C@H]1CN(C(=O)c2cc(NC(=O)[C@@H]3O[C@@](C)(C(F)(F)F)[C@@H](C)[C@H]3c3ccc(F)c(F)c3OC)ccn2)C[C@@H]1OC]

(2R,3S,4S,5R)-3-(3,4-difluoro-2-methoxyphenyl)-N-(2-((3S,4S)-3-
methoxy-4-(methylamino)pyrrolidine-1-carbonyl)pyridin-4-yl)-4,5-
dimethyl-5-(trifluoromethyl)tetrahydrofuran-2-carboxamide

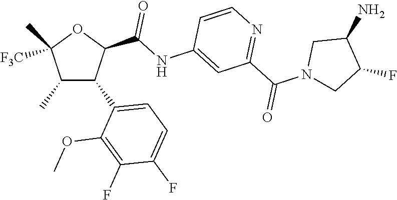 [SMILES: COc1c([C@H]2[C@H](C(=O)Nc3ccnc(C(=O)N4C[C@@H](N)[C@H](F)C4)c3)O[C@@](C)(C(F)(F)F)[C@H]2C)ccc(F)c1F]

(2R,3S,4S,5R)-N-(2-((3R,4R)-3-amino-4-fluoropyrrolidine-1-
carbonyl)pyridin-4-yl)-3-(3,4-difluoro-2-methoxyphenyl)-4,5-
dimethyl-5-(trifluoromethyl)tetrahydrofuran-2-carboxamide

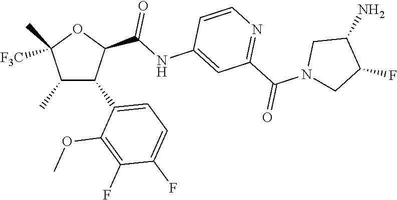 [SMILES: COc1c([C@H]2[C@H](C(=O)Nc3ccnc(C(=O)N4C[C@@H](F)[C@@H](N)C4)c3)O[C@@](C)(C(F)(F)F)[C@H]2C)ccc(F)c1F]

(2R,3S,4S,5R)-N-(2-((3S,4R)-3-amino-4-fluoropyrrolidine-1-
carbonyl)pyridin-4-yl)-3-(3,4-difluoro-2-methoxyphenyl)-4,5-
dimethyl-5-(trifluoromethyl)tetrahydrofuran-2-carboxamide

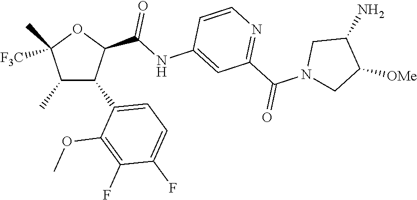 [SMILES: COc1c([C@H]2[C@H](C(=O)Nc3ccnc(C(=O)N4C[C@H](N)[C@H](OC)C4)c3)O[C@@](C)(C(F)(F)F)[C@H]2C)ccc(F)c1F]

(2R,3S,4S,5R)-N-(2-((3S,4R)-3-amino-4-methoxypyrrolidine-1-
carbonyl)pyridin-4-yl)-3-(3,4-difluoro-2-methoxyphenyl)-4,5-
dimethyl-5-(trifluoromethyl)tetrahydrofuran-2-carboxamide

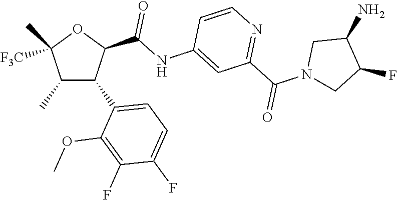 [SMILES: COc1c([C@H]2[C@H](C(=O)Nc3ccnc(C(=O)N4C[C@@H](N)[C@@H](F)C4)c3)O[C@@](C)(C(F)(F)F)[C@H]2C)ccc(F)c1F]

(2R,3S,4S,5R)-N-(2-((3R,4S)-3-amino-4-fluoropyrrolidine-1-
carbonyl)pyridin-4-yl)-3-(3,4-difluoro-2-methoxyphenyl)-4,5-
dimethyl-5-(trifluoromethyl)tetrahydrofuran-2-carboxamide

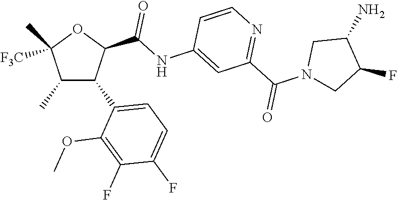 [SMILES: COc1c([C@H]2[C@H](C(=O)Nc3ccnc(C(=O)N4C[C@H](N)[C@@H](F)C4)c3)O[C@@](C)(C(F)(F)F)[C@H]2C)ccc(F)c1F]

(2R,3S,4S,5R)-N-(2-((3S,4S)-3-amino-4-fluoropyrrolidine-1-
carbonyl)pyridin-4-yl)-3-(3,4-difluoro-2-methoxyphenyl)-4,5-
dimethyl-5-(trifluoromethyl)tetrahydrofuran-2-carboxamide

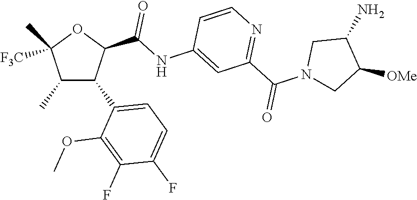 [SMILES: COc1c([C@H]2[C@H](C(=O)Nc3ccnc(C(=O)N4C[C@H](OC)[C@@H](N)C4)c3)O[C@@](C)(C(F)(F)F)[C@H]2C)ccc(F)c1F]

(2R,3S,4S,5R)-N-(2-((3S,4S)-3-amino-4-methoxypyrrolidine-1-
carbonyl)pyridin-4-yl)-3-(3,4-difluoro-2-methoxyphenyl)-4,5-
dimethyl-5-(trifluoromethyl)tetrahydrofuran-2-carboxamide

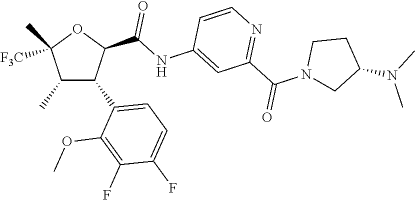 [SMILES: COc1c([C@H]2[C@H](C(=O)Nc3ccnc(C(=O)N4CC[C@H](N(C)C)C4)c3)O[C@@](C)(C(F)(F)F)[C@H]2C)ccc(F)c1F]

(2R,3S,4S,5R)-3-(3,4-difluoro-2-methoxyphenyl)-N-(2-((S)-3-
(dimethylamino)pyrrolidine-1-carbonyl)pyridin-4-yl)-4,5-
dimethyl-5-(trifluoromethyl)tetrahydrofuran-2-carboxamide

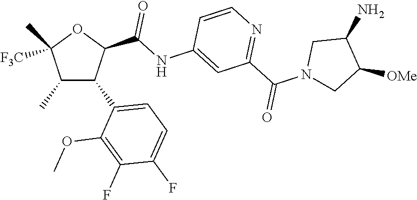 [SMILES: COc1c([C@H]2[C@H](C(=O)Nc3ccnc(C(=O)N4C[C@@H](N)[C@@H](OC)C4)c3)O[C@@](C)(C(F)(F)F)[C@H]2C)ccc(F)c1F]

(2R,3S,4S,5R)-N-(2-((3R,4S)-3-amino-4-methoxypyrrolidine-1-
carbonyl)pyridin-4-yl)-3-(3,4-difluoro-2-methoxyphenyl)-4,5-
dimethyl-5-(trifluoromethyl)tetrahydrofuran-2-carboxamide

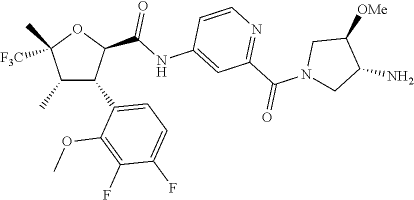

(2R,3S,4S,5R)-N-(2-((3R,4R)-3-amino-4-methoxypyrrolidine-1-
carbonyl)pyridin-4-yl)-3-(3,4-difluoro-2-methoxyphenyl)-4,5-
dimethyl-5-(trifluoromethyl)tetrahydrofuran-2-carboxamide

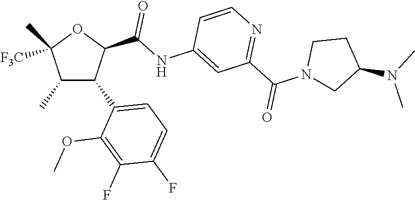 [SMILES: COc1c([C@H]2[C@H](C(=O)Nc3ccnc(C(=O)N4CC[C@@H](N(C)C)C4)c3)O[C@@](C)(C(F)(F)F)[C@H]2C)ccc(F)c1F]

(2R,3S,4S,5R)-3-(3,4-difluoro-2-methoxyphenyl)-N-(2-((R)-3-
(dimethylamino)pyrrolidine-1-carbonyl)pyridin-4-yl)-4,5-dimeth-
yl-5-(trifluoromethyl)tetrahydrofuran-2-carboxamide

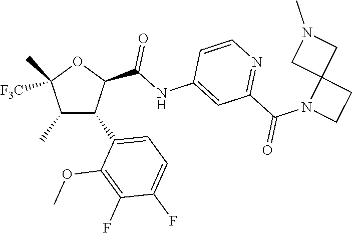 [SMILES: COc1c([C@H]2[C@H](C(=O)Nc3ccnc(C(=O)N4CCC45CN(C)C5)c3)O[C@@](C)(C(F)(F)F)[C@H]2C)ccc(F)c1F]

(2R,3S,4S,5R)-3-(3,4-difluoro-2-methoxyphenyl)-4,5-
dimethyl-N-(2-(6-methyl-1,6-diazaspiro[3.3]heptane-1-
carbonyl)pyridin-4-yl)-5-(trifluoromethyl)tetrahydro-
furan-2-carboxamide

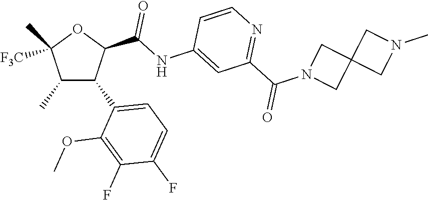 [SMILES: COc1c([C@H]2[C@H](C(=O)Nc3ccnc(C(=O)N4CC5(CN(C)C5)C4)c3)O[C@@](C)(C(F)(F)F)[C@H]2C)ccc(F)c1F]

(2R,3S,4S,5R)-3-(3,4-difluoro-2-methoxyphenyl)-4,5-dimethyl-N-
(2-(6-methyl-2,6-diazaspiro[3.3]heptane-2-carbonyl)pyridin-4-yl)-
5-(trifluoromethyl)tetrahydrofuran-2-carboxamide

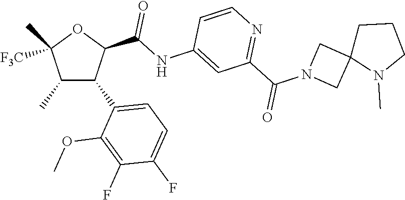 [SMILES: COc1c([C@H]2[C@H](C(=O)Nc3ccnc(C(=O)N4CC5(CCCN5C)C4)c3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(2-(5-methyl-2,5-diazaspiro[3.4]octane-2-carbonyl)pyridin-4-
yl)-5-(trifluoromethyl)tetrahydrofuran-2-carboxamide

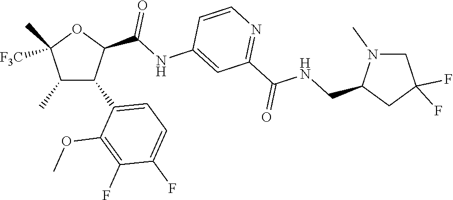 [SMILES: COc1c([C@H]2[C@H](C(=O)Nc3ccnc(C(=O)NC[C@@H]4CC(F)(F)CN4C)c3)O[C@@](C)(C(F)(F)F)[C@H]2C)ccc(F)c1F]

N-(((S)-4,4-difluoro-1-methylpyrrolidin-2-yl)methyl)-4-((2R,3S,4S,5R)-
3-(3,4-difluoro-2-methoxyphenyl)-4,5-dimethyl-5-(trifluoromethyl)tetra-
hydrofuran-2-carboxamido)picolinamide

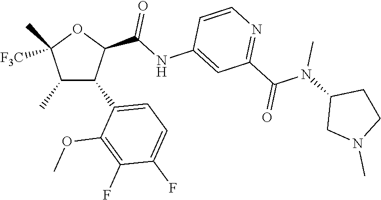 [SMILES: COc1c([C@H]2[C@H](C(=O)Nc3ccnc(C(=O)N(C)[C@@H]4CCN(C)C4)c3)O[C@@](C)(C(F)(F)F)[C@H]2C)ccc(F)c1F]

4-((2R,3S,4S,5R)-3-(3,4-difluoro-2-methoxyphenyl)-4,5-
dimethyl-5-(trifluoromethyl)tetrahydrofuran-2-carboxamido)-
N-methyl-N-((R)-1-methylpyrrolidin-3-yl)picolinamide

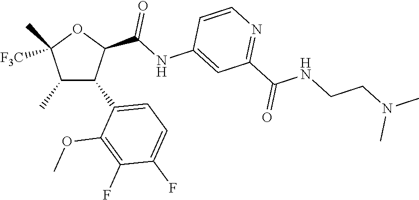 [SMILES: COc1c([C@H]2[C@H](C(=O)Nc3ccnc(C(=O)NCCN(C)C)c3)O[C@@](C)(C(F)(F)F)[C@H]2C)ccc(F)c1F]

4-((2R,3S,4S,5R)-3-(3,4-difluoro-2-methoxyphenyl)-4,5-dimethyl-
5-(trifluoromethyl)tetrahydrofuran-2-carboxamido)-N-(2-(dimethyl-
amino)ethyl)picolinamide

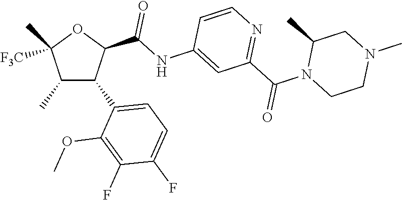 [SMILES: COc1c([C@H]2[C@H](C(=O)Nc3ccnc(C(=O)N4CCN(C)C[C@@H]4C)c3)O[C@@](C)(C(F)(F)F)[C@H]2C)ccc(F)c1F]

(2R,3S,4S,5R)-3-(3,4-difluoro-2-methoxyphenyl)-N-(2-((S)-
2,4-dimethylpiperazine-1-carbonyl)pyridin-4-yl)-4,5-dimethyl-
5-(trifluoromethyl)tetrahydrofuran-2-carboxamide

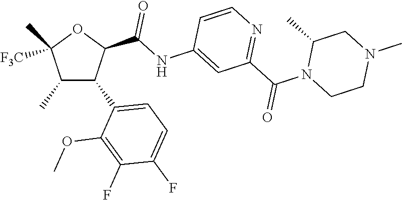 [SMILES: COc1c([C@H]2[C@H](C(=O)Nc3ccnc(C(=O)N4CCN(C)C[C@H]4C)c3)O[C@@](C)(C(F)(F)F)[C@H]2C)ccc(F)c1F]

(2R,3S,4S,5R)-3-(3,4-difluoro-2-methoxyphenyl)-N-(2-((R)-2,4-
dimethylpiperazine-1-carbonyl)pyridin-4-yl)-4,5-dimethyl-5-
(trifluoromethyl)tetrahydrofuran-2-carboxamide

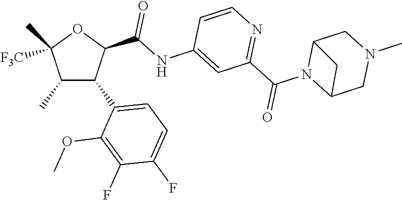 [SMILES: COc1c([C@H]2[C@H](C(=O)Nc3ccnc(C(=O)N4C5CC4CN(C)C5)c3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(2-(3-methyl-3,6-diazabicyclo[3.1.1]heptane-6-carbonyl)-
pyridin-4-yl)-5-(trifluoromethyl)tetrahydrofuran-2-carboxamide

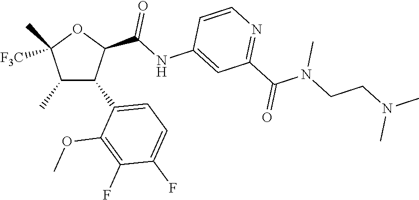 [SMILES: COc1c([C@H]2[C@H](C(=O)Nc3ccnc(C(=O)N(C)CCN(C)C)c3)O[C@@](C)(C(F)(F)F)[C@H]2C)ccc(F)c1F]

4-((2R,3S,4S,5R)-3-(3,4-difluoro-2-methoxyphenyl)-4,5-dimethyl-
5-(trifluoromethyl)tetrahydrofuran-2-carboxamido)-N-(2-(dimethyl-
amino)ethyl)-N-methylpicolinamide

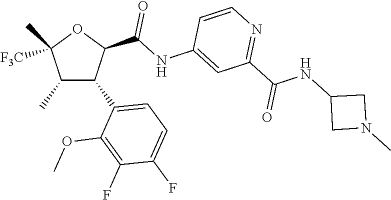 [SMILES: COc1c([C@H]2[C@H](C(=O)Nc3ccnc(C(=O)NC4CN(C)C4)c3)O[C@@](C)(C(F)(F)F)[C@H]2C)ccc(F)c1F]

4-((2R,3S,4S,5R)-3-(3,4-difluoro-2-methoxyphenyl)-4,5-
dimethyl-5-(trifluoromethyl)tetrahydrofuran-2-carboxamido)-
N-(1-methylazetidin-3-yl)picolinamide

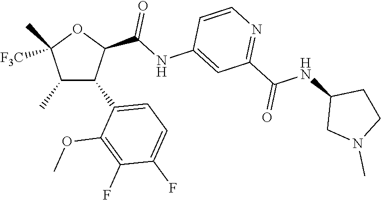 [SMILES: COc1c([C@H]2[C@H](C(=O)Nc3ccnc(C(=O)N[C@H]4CCN(C)C4)c3)O[C@@](C)(C(F)(F)F)[C@H]2C)ccc(F)c1F]

4-((2R,3S,4S,5R)-3-(3,4-difluoro-2-methoxyphenyl)-4,5-
dimethyl-5-(trifluoromethyl)tetrahydrofuran-2-carboxamido)-
N-((S)-1-methylpyrrolidin-3-yl)picolinamide

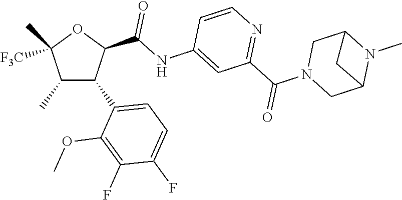 [SMILES: COc1c([C@H]2[C@H](C(=O)Nc3ccnc(C(=O)N4CC5CC(C4)N5C)c3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(2-(6-methyl-3,6-diazabicyclo[3.1.1]heptane-3-carbonyl)-
pyridin-4-yl)-5-(trifluoromethyl)tetrahydrofuran-2-carboxamide

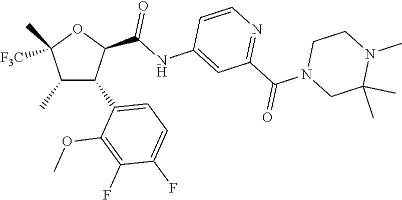 [SMILES: COc1c([C@H]2[C@H](C(=O)Nc3ccnc(C(=O)N4CCN(C)C(C)(C)C4)c3)O[C@@](C)(C(F)(F)F)[C@H]2C)ccc(F)c1F]

(2R,3S,4S,5R)-3-(3,4-difluoro-2-methoxyphenyl)-4,5-dimethyl-
5-(trifluoromethyl)-N-(2-(3,3,4-trimethylpiperazine-1-carbonyl)
pyridin-4-yl)tetrahydrofuran-2-carboxamide

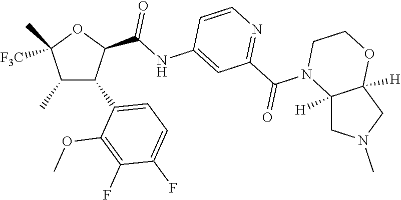 [SMILES: COc1c([C@H]2[C@H](C(=O)Nc3ccnc(C(=O)N4CCO[C@H]5CN(C)C[C@H]54)c3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(2-((4aR,7aS)-6-methyloctahydropyrrolo[3,4-b][1,4]oxazine-
4-carbonyl)pyridin-4-yl)-5-(trifluoromethyl)tetrahydrofuran-
2-carboxamide

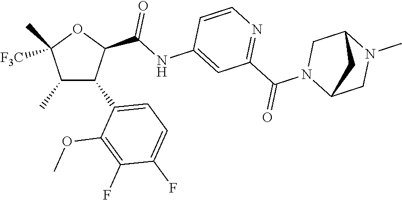 [SMILES: COc1c([C@H]2[C@H](C(=O)Nc3ccnc(C(=O)N4C[C@H]5C[C@@H]4CN5C)c3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(2-((1R,4R)-5-methyl-2,5-diazabicyclo[2.2.1]heptane-2-
carbonyl)pyridin-4-yl)-5-(trifluoromethyl)tetrahydrofuran-
2-carboxamide

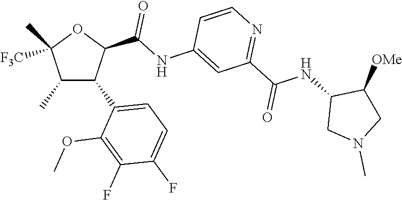 [SMILES: COc1c([C@H]2[C@H](C(=O)Nc3ccnc(C(=O)N[C@H]4CN(C)C[C@@H]4OC)c3)O[C@@](C)(C(F)(F)F)[C@H]2C)ccc(F)c1F]

4-((2R,3S,4S,5R)-3-(3,4-difluoro-2-methoxyphenyl)-4,5-dimeth-
yl-5-(trifluoromethyl)tetrahydrofuran-2-carboxamido)-N-
((3S,4S)-4-methoxy-1-methylpyrrolidin-3-yl)picolinamide

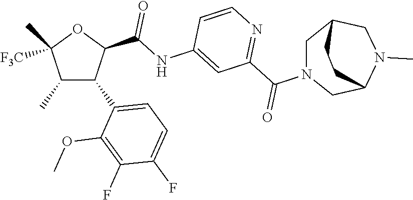 [SMILES: COc1c([C@H]2[C@H](C(=O)Nc3ccnc(C(=O)N4C[C@H]5CC[C@@H](C4)N(C)C5)c3)O[C@@](C)(C(F)(F)F)[C@H]2C)ccc(F)c1F]

(2R,3S,4S,5R)-3-(3,4-difluoro-2-methoxyphenyl)-4,5-dimethyl-N-
(2-((1S,5S)-6-methyl-3,6-diazabicyclo[3.2.2]nonane-3-carbonyl)-
pyridin-4-yl)-5-(trifluoromethyl)tetrahydrofuran-2-carboxamide

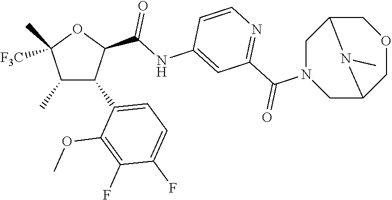 [SMILES: COc1c([C@H]2[C@H](C(=O)Nc3ccnc(C(=O)N4CC5COCC(C4)N5C)c3)O[C@@](C)(C(F)(F)F)[C@H]2C)ccc(F)c1F]

(2R,3S,4S,5R)-3-(3,4-difluoro-2-methoxyphenyl)-4,5-dimethyl-
N-(2-(9-methyl-3-oxa-7,9-diazabicyclo[3.3.1]nonane-7-
carbonyl)pyridin-4-yl)-5-(trifluoromethyl)tetrahydrofuran-
2-carboxamide

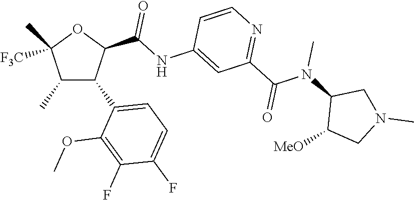 [SMILES: COc1c([C@H]2[C@H](C(=O)Nc3ccnc(C(=O)N(C)[C@H]4CN(C)C[C@@H]4OC)c3)O[C@@](C)(C(F)(F)F)[C@H]2C)ccc(F)c1F]

4-((2R,3S,4S,5R)-3-(3,4-difluoro-2-methoxyphenyl)-4,5-dimethyl-
5-(trifluoromethyl)tetrahydrofuran-2-carboxamido)-N-((3S,4S)-4-
methoxy-1-methylpyrrolidin-3-yl)-N-methylpicolinamide

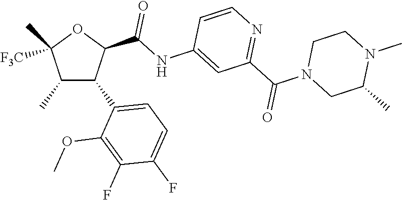 [SMILES: COc1c([C@H]2[C@H](C(=O)Nc3ccnc(C(=O)N4CCN(C)[C@H](C)C4)c3)O[C@@](C)(C(F)(F)F)[C@H]2C)ccc(F)c1F]

(2R,3S,4S,5R)-3-(3,4-difluoro-2-methoxyphenyl)-N-(2-((R)-3,4-
dimethylpiperazine-1-carbonyl)pyridin-4-yl)-4,5-dimethyl-5-
(trifluoromethyl)tetrahydrofuran-2-carboxamide

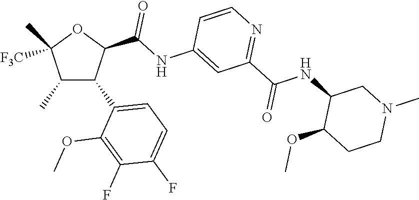 [SMILES: COc1c([C@H]2[C@H](C(=O)Nc3ccnc(C(=O)N[C@H]4CN(C)CC[C@H]4OC)c3)O[C@@](C)(C(F)(F)F)[C@H]2C)ccc(F)c1F]

4-((2R,3S,4S,5R)-3-(3,4-difluoro-2-methoxyphenyl)-4,5-dimethyl-
5-(trifluoromethyl)tetrahydrofuran-2-carboxamido)-N-((3S,4R)-4-
methoxy-1-methylpiperidin-3-yl)picolinamide

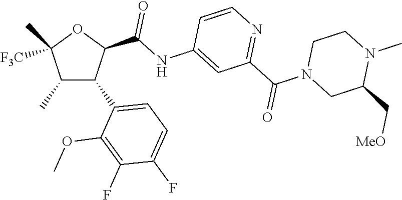 [SMILES: COC[C@H]1CN(C(=O)c2cc(NC(=O)[C@@H]3O[C@@](C)(C(F)(F)F)[C@@H](C)[C@H]3c3ccc(F)c(F)c3OC)ccn2)CCN1C]

(2R,3S,4S,5R)-3-(3,4-difluoro-2-methoxyphenyl)-N-(2-((R)-3-
(methoxymethyl)-4-methylpiperazine-1-carbonyl)pyridin-4-yl)-
4,5-dimethyl-5-(trifluoromethyl)tetrahydrofuran-2-carboxamide

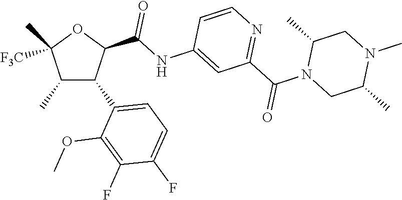 [SMILES: COc1c([C@H]2[C@H](C(=O)Nc3ccnc(C(=O)N4C[C@@H](C)N(C)C[C@H]4C)c3)O[C@@](C)(C(F)(F)F)[C@H]2C)ccc(F)c1F]

(2R,3S,4S,5R)-3-(3,4-difluoro-2-methoxyphenyl)-4,5-dimethyl-
5-(trifluoromethyl)-N-(2-((2R,5R)-2,4,5-trimethylpiperazine-1-
carbonyl)pyridin-4-yl)tetrahydrofuran-2-carboxamide

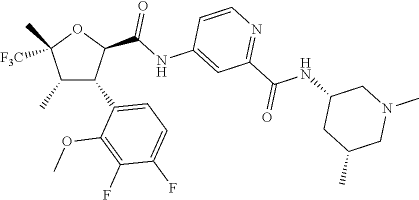 [SMILES: COc1c([C@H]2[C@H](C(=O)Nc3ccnc(C(=O)N[C@H]4C[C@@H](C)CN(C)C4)c3)O[C@@](C)(C(F)(F)F)[C@H]2C)ccc(F)c1F]

4-((2R,3S,4S,5R)-3-(3,4-difluoro-2-methoxyphenyl)-4,5-dimethyl-
5-(trifluoromethyl)tetrahydrofuran-2-carboxamido)-N-((3S,5R)-
1,5-dimethylpiperidin-3-yl)picolinamide

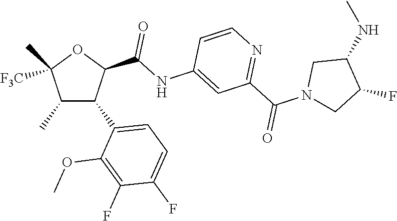 [SMILES: CN[C@H]1CN(C(=O)c2cc(NC(=O)[C@@H]3O[C@@](C)(C(F)(F)F)[C@@H](C)[C@H]3c3ccc(F)c(F)c3OC)ccn2)C[C@H]1F]

(2R,3S,4S,5R)-3-(3,4-difluoro-2-methoxyphenyl)-N-(2-((3R,4S)-
3-fluoro-4-(methylamino)pyrrolidine-1-carbonyl)pyridin-4-yl)-
4,5-dimethyl-5-(trifluoromethyl)tetrahydrofuran-2-carboxamide

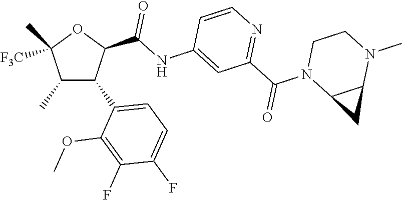 [SMILES: COc1c([C@H]2[C@H](C(=O)Nc3ccnc(C(=O)N4CCN(C)[C@H]5C[C@H]54)c3)O[C@@](C)(C(F)(F)F)[C@H]2C)ccc(F)c1F]

(2R,3S,4S,5R)-3-(3,4-difluoro-2-methoxyphenyl)-4,5-dimeth-
yl-N-(2-((1R,6S)-5-methyl-2,5-diazabicyclo[4.1.0]heptane-2-
carbonyl)pyridin-4-yl)-5-(trifluoromethyl)tetrahydrofuran-
2-carboxamide

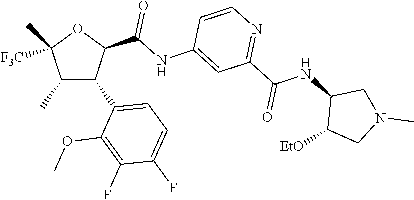 [SMILES: CCO[C@H]1CN(C)C[C@@H]1NC(=O)c1cc(NC(=O)[C@@H]2O[C@@](C)(C(F)(F)F)[C@@H](C)[C@H]2c2ccc(F)c(F)c2OC)ccn1]

4-((2R,3S,4S,5R)-3-(3,4-difluoro-2-methoxyphenyl)-4,5-dimethyl-
5-(trifluoromethyl)tetrahydrofuran-2-carboxamido)-N-((3S,4S)-4-
ethoxy-1-methylpyrrolidin-3-yl)picolinamide

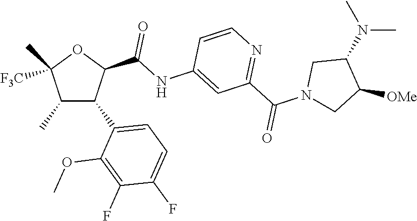 [SMILES: COc1c([C@H]2[C@H](C(=O)Nc3ccnc(C(=O)N4C[C@H](OC)[C@@H](N(C)C)C4)c3)O[C@@](C)(C(F)(F)F)[C@H]2C)ccc(F)c1F]

(2R,3S,4S,5R)-3-(3,4-difluoro-2-methoxyphenyl)-N-(2-((3S,4S)-3-
(dimethylamino)-4-methoxypyrrolidine-1-carbonyl)pyridin-4-yl)-
4,5-dimethyl-5-(trifluoromethyl)tetrahydrofuran-2-carboxamide

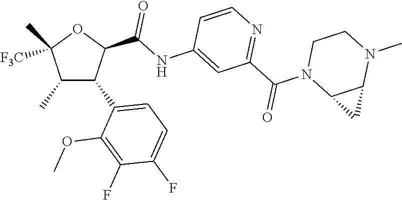 [SMILES: COc1c([C@H]2[C@H](C(=O)Nc3ccnc(C(=O)N4CCN(C)[C@@H]5C[C@@H]54)c3)O[C@@](C)(C(F)(F)F)[C@H]2C)ccc(F)c1F]

(2R,3S,4S,5R)-3-(3,4-difluoro-2-methoxyphenyl)-4,5-dimethyl-N-
(2-((1S,6R)-5-methyl-2,5-diazabicyclo[4.1.0]heptane-2-carbonyl)
pyridin-4-yl)-5-(trifluoromethyl)tetrahydrofuran-2-carboxamide

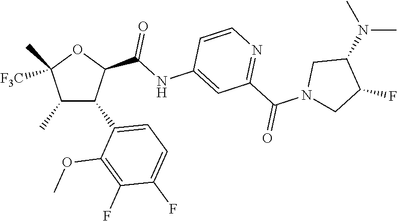 [SMILES: COc1c([C@H]2[C@H](C(=O)Nc3ccnc(C(=O)N4C[C@@H](F)[C@@H](N(C)C)C4)c3)O[C@@](C)(C(F)(F)F)[C@H]2C)ccc(F)c1F]

(2R,3S,4S,5R)-3-(3,4-difluoro-2-methoxyphenyl)-N-(2-((3S,4R)-
3-(dimethylamino)-4-fluoropyrrolidine-1-carbonyl)pyridin-4-yl)-
4,5-dimethyl-5-(trifluoromethyl)tetrahydrofuran-2-carboxamide

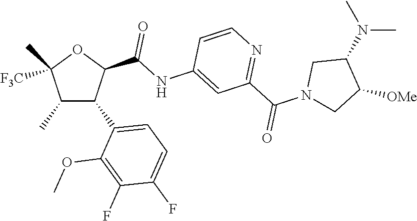 [SMILES: COc1c([C@H]2[C@H](C(=O)Nc3ccnc(C(=O)N4C[C@@H](OC)[C@@H](N(C)C)C4)c3)O[C@@](C)(C(F)(F)F)[C@H]2C)ccc(F)c1F]

(2R,3S,4S,5R)-3-(3,4-difluoro-2-methoxyphenyl)-N-(2-((3S,4R)-3-
(dimethylamino)-4-methoxypyrrolidine-1-carbonyl)pyridin-4-yl)-
4,5-dimethyl-5-(trifluoromethyl)tetrahydrofuran-2-carboxamide

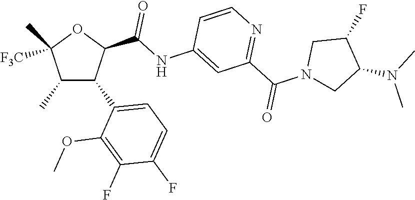 [SMILES: COc1c([C@H]2[C@H](C(=O)Nc3ccnc(C(=O)N4C[C@@H](N(C)C)[C@@H](F)C4)c3)O[C@@](C)(C(F)(F)F)[C@H]2C)ccc(F)c1F]

(2R,3S,4S,5R)-3-(3,4-difluoro-2-methoxyphenyl)-N-(2-((3R,4S)-3-
(dimethylamino)-4-fluoropyrrolidine-1-carbonyl)pyridin-4-yl)-4,5-
dimethyl-5-(trifluoromethyl)tetrahydrofuran-2-carboxamide

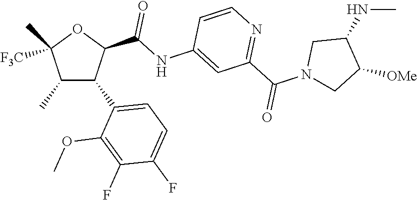 [SMILES: CN[C@H]1CN(C(=O)c2cc(NC(=O)[C@@H]3O[C@@](C)(C(F)(F)F)[C@@H](C)[C@H]3c3ccc(F)c(F)c3OC)ccn2)C[C@H]1OC]

(2R,3S,4S,5R)-3-(3,4-difluoro-2-methoxyphenyl)-N-(2-((3R,4S)-
3-methoxy-4-(methylamino)pyrrolidine-1-carbonyl)pyridin-4-yl)-
4,5-dimethyl-5-(trifluoromethyl)tetrahydrofuran-2-carboxamide

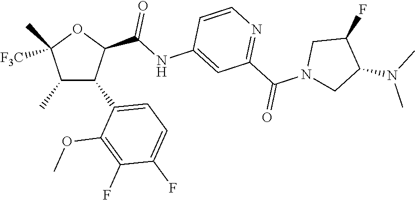 [SMILES: COc1c([C@H]2[C@H](C(=O)Nc3ccnc(C(=O)N4C[C@@H](F)[C@H](N(C)C)C4)c3)O[C@@](C)(C(F)(F)F)[C@H]2C)ccc(F)c1F]

(2R,3S,4S,5R)-3-(3,4-difluoro-2-methoxyphenyl)-N-(2-((3R,4R)-3-
(dimethylamino)-4-fluoropyrrolidine-1-carbonyl)pyridin-4-yl)-4,5-
dimethyl-5-(trifluoromethyl)tetrahydrofuran-2-carboxamide

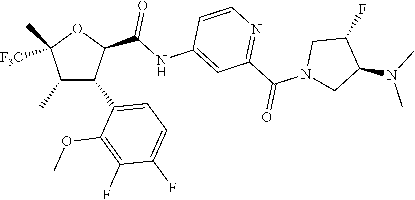 [SMILES: COc1c([C@H]2[C@H](C(=O)Nc3ccnc(C(=O)N4C[C@H](F)[C@@H](N(C)C)C4)c3)O[C@@](C)(C(F)(F)F)[C@H]2C)ccc(F)c1F]

(2R,3S,4S,5R)-3-(3,4-difluoro-2-methoxyphenyl)-N-(2-((3S,4S)-3-
(dimethylamino)-4-fluoropyrrolidine-1-carbonyl)pyridin-4-yl)-4,5-
dimethyl-5-(trifluoromethyl)tetrahydrofuran-2-carboxamide

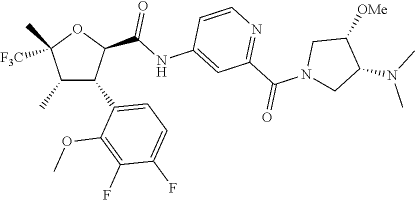

(2R,3S,4S,5R)-3-(3,4-difluoro-2-methoxyphenyl)-N-(2-((3R,4S)-3-
(dimethylamino)-4-methoxypyrrolidine-1-carbonyl)pyridin-4-yl)-
4,5-dimethyl-5-(trifluoromethyl)tetrahydrofuran-2-carboxamide

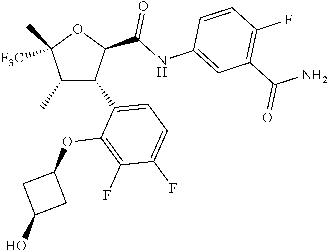 [SMILES: C[C@H]1[C@@H](c2ccc(F)c(F)c2O[C@H]2C[C@@H](O)C2)[C@H](C(=O)Nc2ccc(F)c(C(N)=O)c2)O[C@@]1(C)C(F)(F)F]

(2R,3S,4S,5R)-N-(3-carbamoyl-4-fluorophenyl)-3-
(3,4-difluoro-2-((1s,3R)-3-hydroxycyclobutoxy)-
phenyl)-4,5-dimethyl-5-(trifluoromethyl)tetrahydro-
furan-2-carboxamide

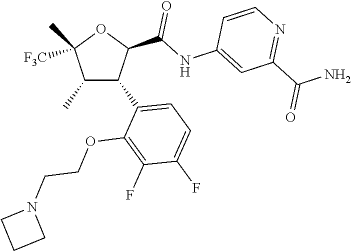 [SMILES: C[C@H]1[C@@H](c2ccc(F)c(F)c2OCCN2CCC2)[C@H](C(=O)Nc2ccnc(C(N)=O)c2)O[C@@]1(C)C(F)(F)F]

4-((2R,3S,4S,5R)-3-(2-(2-(azetidin-1-yl)ethoxy)-3,4-
difluorophenyl)-4,5-dimethyl-5-(trifluoromethyl)tetra-
hydrofuran-2-carboxamido)picolinamide
(BLANK)

In some embodiments, the invention relates to a compound selected from Table B, or a pharmaceutically acceptable salt thereof. In other embodiments, the invention relates to a compound selected from Table B, i.e., the compound in non-salt form.

TABLE B
Compound Structures and Names.

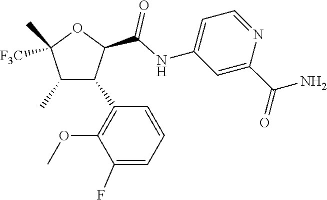 [SMILES: COc1c(F)cccc1[C@H]1[C@H](C(=O)Nc2ccnc(C(N)=O)c2)O[C@@](C)(C(F)(F)F)[C@H]1C]

4-((2R,3S,4S,5R)-3-(3-fluoro-2-
methoxyphenyl)-4,5-dimethyl-5-
(trifluoromethyl)tetrahydrofuran-2-
carboxamido)picolinamide

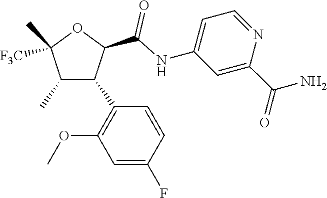 [SMILES: COc1cc(F)ccc1[C@H]1[C@H](C(=O)Nc2ccnc(C(N)=O)c2)O[C@@](C)(C(F)(F)F)[C@H]1C]

4-((2R,3S,4S,5R)-3-(4-fluoro-2-
methoxyphenyl)-4,5-dimethyl-5-
(trifluoromethyl)tetrahydrofuran-2-
carboxamido)picolinamide

In some embodiments, the invention relates to a compound selected from Table A or Table B, or a pharmaceutically acceptable salt thereof. In other embodiments, the invention relates to a compound selected from Table A or Table B, i.e., the compound in non-salt form.

In some embodiments, the invention relates to a compound of formula

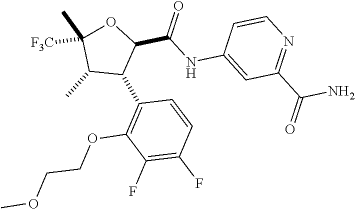 [SMILES: COCCOc1c([C@H]2[C@H](C(=O)Nc3ccnc(C(N)=O)c3)O[C@@](C)(C(F)(F)F)[C@H]2C)ccc(F)c1F]

or a pharmaceutically acceptable salt thereof, wherein the compound has the absolute stereochemistry of the second eluting isomer when rac-4-((2R,3S,4S,5R)-3-(3,4-difluoro-2-(2-methoxyethoxy)phenyl)-4,5-dimethyl-5-(trifluoromethyl)tetrahydrofuran-2-carboxamido) picolinamide is separated by SFC as described in Example 1. In other embodiments, the invention relates to the foregoing compound in non-salt form. Such compound is considered to be a “compound of the invention,” as that term is used herein.

In some embodiments, the invention relates to a compound of formula

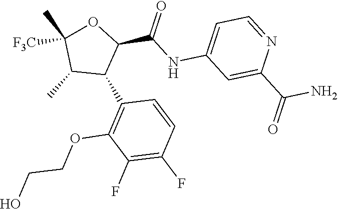 [SMILES: C[C@H]1[C@@H](c2ccc(F)c(F)c2OCCO)[C@H](C(=O)Nc2ccnc(C(N)=O)c2)O[C@@]1(C)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

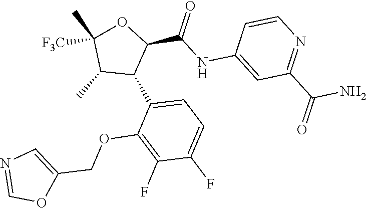 [SMILES: C[C@H]1[C@@H](c2ccc(F)c(F)c2OCc2cnco2)[C@H](C(=O)Nc2ccnc(C(N)=O)c2)O[C@@]1(C)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

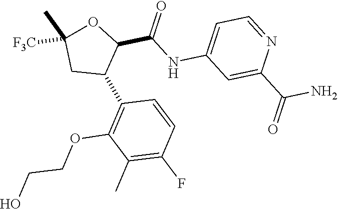 [SMILES: Cc1c(F)ccc([C@@H]2C[C@](C)(C(F)(F)F)O[C@H]2C(=O)Nc2ccnc(C(N)=O)c2)c1OCCO]

or a pharmaceutically acceptable salt thereof, wherein the compound has the absolute stereochemistry of the second eluting isomer when rac-4-((2R,3S,5R)-3-(4-fluoro-2-(2-hydroxyethoxy)-3-methylphenyl)-5-methyl-5-(trifluoromethyl)tetrahydrofuran-2-carboxamido) picolinamide is separated by SFC as described in Example 5. In other embodiments, the invention relates to the foregoing compound in non-salt form. Such compound is considered to be a “compound of the invention,” as that term is used herein.

In some embodiments, the invention relates to a compound of formula

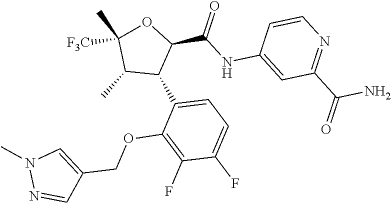 [SMILES: C[C@H]1[C@@H](c2ccc(F)c(F)c2OCc2cnn(C)c2)[C@H](C(=O)Nc2ccnc(C(N)=O)c2)O[C@@]1(C)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

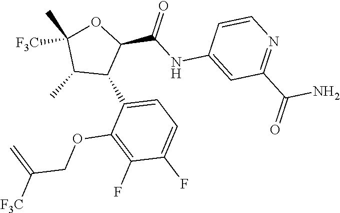 [SMILES: C=C(COc1c([C@H]2[C@H](C(=O)Nc3ccnc(C(N)=O)c3)O[C@@](C)(C(F)(F)F)[C@H]2C)ccc(F)c1F)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

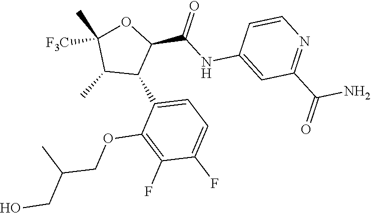 [SMILES: CC(CO)COc1c([C@H]2[C@H](C(=O)Nc3ccnc(C(N)=O)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

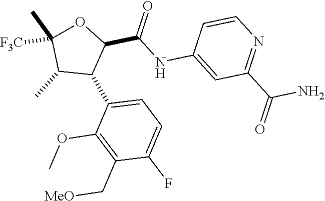 [SMILES: COCc1c(F)ccc([C@H]2[C@H](C(=O)Nc3ccnc(C(N)=O)c3)O[C@@](C)(C(F)(F)F)[C@H]2C)c1OC]

or a pharmaceutically acceptable salt thereof, wherein the compound has the absolute stereochemistry of the second eluting isomer when rac-4-((2R,3S,4S,5R)-3-(4-fluoro-2-methoxy-3-(methoxymethyl)phenyl)-4,5-dimethyl-5-(trifluoromethyl)tetrahydrofuran-2-carboxamido) picolinamide is separated by SFC as described in Example 13. In other embodiments, the invention relates to the foregoing compound in non-salt form. Such compound is considered to be a “compound of the invention,” as that term is used herein.

In some embodiments, the invention relates to a compound of formula

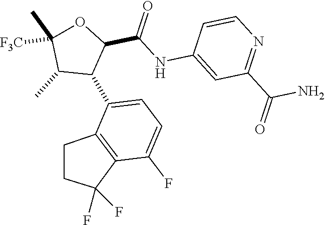 [SMILES: C[C@H]1[C@@H](c2ccc(F)c3c2CCC3(F)F)[C@H](C(=O)Nc2ccnc(C(N)=O)c2)O[C@@]1(C)C(F)(F)F]

or a pharmaceutically acceptable salt thereof, wherein the compound has the absolute stereochemistry of the third eluting isomer when a mixture of rac-4-((2R,3S,4S,5R)-4,5-dimethyl-3-(1,1,7-trifluoro-2,3-dihydro-1H-inden-4-yl)-5-(trifluoromethyl)tetrahydrofuran-2-carboxamido) picolinamide and rac-4-((2R,3S,4R,5S)-4,5-dimethyl-3-(1,1,7-trifluoro-2,3-dihydro-1H-inden-4-yl)-5-(trifluoromethyl)tetrahydrofuran-2-carboxamido) picolinamide is separated by SFC as described in Example 13. In other embodiments, the invention relates to the foregoing compound in non-salt form. Such compound is considered to be a “compound of the invention,” as that term is used herein.

In some embodiments, the invention relates to a compound of formula

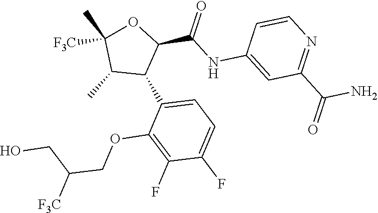 [SMILES: C[C@H]1[C@@H](c2ccc(F)c(F)c2OCC(CO)C(F)(F)F)[C@H](C(=O)Nc2ccnc(C(N)=O)c2)O[C@@]1(C)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

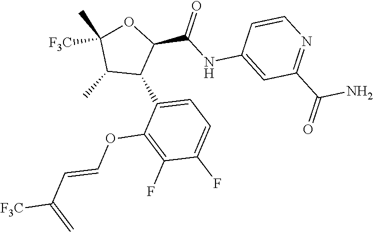 [SMILES: C=C(/C=C/Oc1c([C@H]2[C@H](C(=O)Nc3ccnc(C(N)=O)c3)O[C@@](C)(C(F)(F)F)[C@H]2C)ccc(F)c1F)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

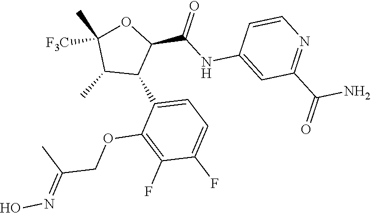 [SMILES: C/C(COc1c([C@H]2[C@H](C(=O)Nc3ccnc(C(N)=O)c3)O[C@@](C)(C(F)(F)F)[C@H]2C)ccc(F)c1F)=N\O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

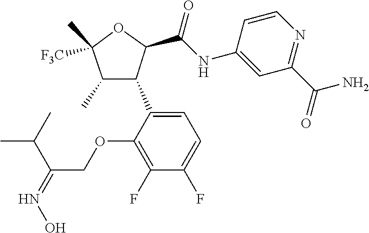 [SMILES: CC(C)/C(COc1c([C@H]2[C@H](C(=O)Nc3ccnc(C(N)=O)c3)O[C@@](C)(C(F)(F)F)[C@H]2C)ccc(F)c1F)=N/O]

or a pharmaceutically acceptable salt thereof, wherein the compound has the absolute stereochemistry of the first eluting isomer when a mixture of geometric isomers of 4-((2R,3S,4S,5R)-3-(3,4-difluoro-2-(2-(hydroxyimino)-3-methylbutoxy)phenyl)-4,5-dimethyl-5-(trifluoromethyl)tetrahydrofuran-2-carboxamido) picolinamide, comprising 4-((2R,3S,4S,5R)-3-(3,4-difluoro-2-((E)-2-(hydroxyimino)-3-methylbutoxy)phenyl)-4,5-dimethyl-5-(trifluoromethyl)tetrahydrofuran-2-carboxamido) picolinamide and 4-((2R,3S,4S,5R)-3-(3,4-difluoro-2-((Z)-2-(hydroxyimino)-3-methylbutoxy)phenyl)-4,5-dimethyl-5-(trifluoromethyl)tetrahydrofuran-2-carboxamido) picolinamide, is separated by SFC as described in Example 21. In other embodiments, the invention relates to the foregoing compound in non-salt form. Such compound is considered to be a “compound of the invention,” as that term is used herein.

In some embodiments, the invention relates to a compound of formula

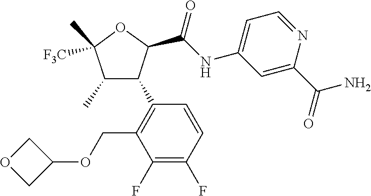 [SMILES: C[C@H]1[C@@H](c2ccc(F)c(F)c2COC2COC2)[C@H](C(=O)Nc2ccnc(C(N)=O)c2)O[C@@]1(C)C(F)(F)F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

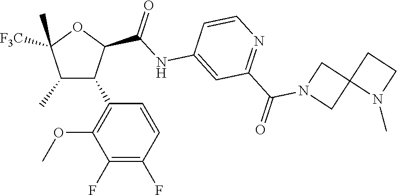 [SMILES: COc1c([C@H]2[C@H](C(=O)Nc3ccnc(C(=O)N4CC5(CCN5C)C4)c3)O[C@@](C)(C(F)(F)F)[C@H]2C)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

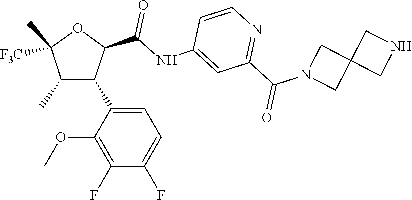 [SMILES: COc1c([C@H]2[C@H](C(=O)Nc3ccnc(C(=O)N4CC5(CNC5)C4)c3)O[C@@](C)(C(F)(F)F)[C@H]2C)ccc(F)c1F]

or a pharmaceutically acceptable salt thereof. In other embodiments, the invention relates to the foregoing compound in non-salt form. In other embodiments, the invention relates to a trifluoroacetate salt of the foregoing compound. Such compound is considered to be a “compound of the invention,” as that term is used herein.

### Salts, Compositions, Uses, Formulation, Administration and Additional Agents

### Pharmaceutically Acceptable Salts and Compositions

As discussed herein, the invention provides compounds, and pharmaceutically acceptable salts thereof, that are inhibitors of voltage-gated sodium channels, and thus the present compounds, and pharmaceutically acceptable salts thereof, are useful for the treatment of diseases, disorders, and conditions including, but not limited to chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia. Accordingly, in another aspect of the invention, pharmaceutical compositions are provided, wherein these compositions comprise a compound as described herein, or a pharmaceutically acceptable salt thereof, and optionally comprise a pharmaceutically acceptable carrier, adjuvant or vehicle. In certain embodiments, these compositions optionally further comprise one or more additional therapeutic agents. In some embodiments, the additional therapeutic agent is a sodium channel inhibitor.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” of a compound of this invention includes any non-toxic salt that, upon administration to a recipient, is capable of providing, either directly or indirectly, a compound of this invention or an inhibitorily active metabolite or residue thereof. The salt may be in pure form, in a mixture (e.g., solution, suspension, or colloid) with one or more other substances, or in the form of a hydrate, solvate, or co-crystal. As used herein, the term “inhibitorily active metabolite or residue thereof” means that a metabolite or residue thereof is also an inhibitor of a voltage-gated sodium channel.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of the compound of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described herein, the pharmaceutically acceptable compositions of the invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose, starches such as corn starch and potato starch, cellulose and its derivatives such as sodium carboxymethyl cellulose, ethyl cellulose and cellulose acetate, powdered tragacanth, malt, gelatin, talc, excipients such as cocoa butter and suppository waxes, oils such as peanut oil, cottonseed oil, safflower oil, sesame oil, olive oil, corn oil and soybean oil, glycols, such a propylene glycol or polyethylene glycol, esters such as ethyl oleate and ethyl laurate, agar, buffering agents such as magnesium hydroxide and aluminum hydroxide, alginic acid, pyrogen-free water, isotonic saline, Ringer's solution, ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In another aspect, the invention features a pharmaceutical composition comprising a compound of the invention, or a pharmaceutically acceptable salt thereof, and a pharmaceutically acceptable carrier.

In another aspect, the invention features a pharmaceutical composition comprising a therapeutically effective amount of a compound, or a pharmaceutically acceptable salt thereof, and one or more pharmaceutically acceptable carriers or vehicles.

### Uses of Compounds and Pharmaceutically Acceptable Salts and Compositions

In another aspect, the invention features a method of inhibiting a voltage-gated sodium channel in a subject comprising administering to the subject a compound of the invention or a pharmaceutically acceptable salt thereof or a pharmaceutical composition thereof. In another aspect, the voltage-gated sodium channel is Naᵥ1.8.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of gut pain, wherein gut pain comprises inflammatory bowel disease pain, Crohn's disease pain or interstitial cystitis pain wherein said method comprises administering an effective amount of a compound of the invention, a pharmaceutically acceptable salt thereof or a pharmaceutical composition thereof.

In yet another aspect, the invention features a method of treating or lessening the severity in a subject of neuropathic pain comprising administering an effective amount of a compound of the invention, a pharmaceutically acceptable salt thereof or a pharmaceutical composition thereof. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In yet another aspect, the invention features a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion injury, complex regional pain syndrome, drug therapy induced neuralgia, cancer chemotherapy induced neuralgia, anti-retroviral therapy induced neuralgia, post spinal cord injury pain, small fiber neuropathy, idiopathic small-fiber neuropathy, idiopathic sensory neuropathy or trigeminal autonomic cephalalgia wherein said method comprises administering an effective amount of a compound of the invention, a pharmaceutically acceptable salt thereof or a pharmaceutical composition thereof.

In yet another aspect, the invention features a method of treating or lessening the severity in a subject of musculoskeletal pain comprising administering an effective amount of a compound of the invention, a pharmaceutically acceptable salt thereof or a pharmaceutical composition thereof. In some aspects, the musculoskeletal pain comprises osteoarthritis pain.

In yet another aspect, the invention features a method of treating or lessening the severity in a subject of musculoskeletal pain, wherein musculoskeletal pain comprises osteoarthritis pain, back pain, cold pain, burn pain or dental pain wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain or vulvodynia wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises fibromyalgia pain wherein said method comprises administering an effective amount of a compound of the invention, a pharmaceutically acceptable salt thereof or a pharmaceutical composition thereof.

In yet another aspect, the invention features a method of treating or lessening the severity in a subject of pathological cough wherein said method comprises administering an effective amount of a compound of the invention, a pharmaceutically acceptable salt thereof or a pharmaceutical composition thereof.

In yet another aspect, the invention features a method of treating or lessening the severity in a subject of acute pain comprising administering an effective amount of a compound of the invention, a pharmaceutically acceptable salt thereof or a pharmaceutical composition thereof. In some aspects, the acute pain comprises acute post-operative pain.

In yet another aspect, the invention features a method of treating or lessening the severity in a subject of postsurgical pain (e.g., joint replacement pain, soft tissue surgery pain, herniorrhaphy pain, bunionectomy pain or abdominoplasty pain) comprising administering an effective amount of a compound of the invention, a pharmaceutically acceptable salt thereof or a pharmaceutical composition thereof.

In yet another aspect, the invention features a method of treating or lessening the severity in a subject of bunionectomy pain comprising administering an effective amount of a compound of the invention, a pharmaceutically acceptable salt thereof or a pharmaceutical composition thereof.

In yet another aspect, the invention features a method of treating or lessening the severity in a subject of herniorrhaphy pain comprising administering an effective amount of a compound of the invention, a pharmaceutically acceptable salt thereof or a pharmaceutical composition thereof.

In yet another aspect, the invention features a method of treating or lessening the severity in a subject of abdominoplasty pain comprising administering an effective amount of a compound of the invention, a pharmaceutically acceptable salt thereof or a pharmaceutical composition thereof.

In yet another aspect, the invention features a method of treating or lessening the severity in a subject of visceral pain comprising administering an effective amount of a compound of the invention, a pharmaceutically acceptable salt thereof or a pharmaceutical composition thereof. In some aspects, the visceral pain comprises visceral pain from abdominoplasty.

In yet another aspect, the invention features a method of treating or lessening the severity in a subject of a neurodegenerative disease comprising administering an effective amount of a compound of the invention, a pharmaceutically acceptable salt thereof or a pharmaceutical composition thereof. In some aspects, the neurodegenerative disease comprises multiple sclerosis. In some aspects, the neurodegenerative disease comprises Pitt Hopkins Syndrome (PTHS).

In yet another aspect, the invention features a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Naᵥ1.8.

In another aspect, the invention features a method of treating or lessening the severity in a subject of acute pain, sub-acute and chronic pain, nociceptive pain, neuropathic pain, inflammatory pain, nociplastic pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility, comprising administering an effective amount of a compound of the invention, a pharmaceutically acceptable salt thereof or a pharmaceutical composition thereof.

In another aspect, the invention features a method of treating or lessening the severity in a subject of femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache, cluster headaches, chronic and acute neuropathic pain, post-herpetic neuralgia, diabetic neuropathy, HIV-associated neuropathy, trigeminal neuralgia, Charcot-Marie-Tooth neuropathy, hereditary sensory neuropathy, peripheral nerve injury, painful neuromas, ectopic proximal and distal discharges, radiculopathy, chemotherapy induced neuropathic pain, radiotherapy-induced neuropathic pain, persistent/chronic post-surgical pain (e.g., post amputation, post-thoracotomy, post-cardiac surgery), post-mastectomy pain, central pain, spinal cord injury pain, post-stroke pain, thalamic pain, phantom pain (e.g., following removal of lower extremity, upper extremity, breast), intractable pain, acute pain, acute post-operative pain, acute musculoskeletal pain, joint pain, mechanical low back pain, neck pain, tendonitis, injury pain, exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory pain, burn pain, trauma pain, acute intermittent pain, endometriosis, acute herpes zoster pain, sickle cell anemia, acute pancreatitis, breakthrough pain, orofacial pain, sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, urinary incontinence, pathological cough, hyperactive bladder, painful bladder syndrome, interstitial cystitis (IC), prostatitis, complex regional pain syndrome (CRPS), type I, complex regional pain syndrome (CRPS) type II, widespread pain, paroxysmal extreme pain, pruritus, tinnitus, or angina-induced pain, comprising administering an effective amount of a compound of the invention, a pharmaceutically acceptable salt thereof or a pharmaceutical composition thereof.

### Compounds, Pharmaceutically Acceptable Salts, and Compositions for Use

In another aspect, the invention features a compound of the invention, or a pharmaceutically acceptable salt or pharmaceutical composition thereof, for use as a medicament.

In another aspect, the invention features a compound of the invention, or a pharmaceutically acceptable salt or pharmaceutical composition thereof, for use in a method of inhibiting a voltage-gated sodium channel in a subject. In another aspect, the voltage-gated sodium channel is Naᵥ1.8.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In another aspect, the invention features a compound of the invention, or a pharmaceutically acceptable salt or pharmaceutical composition thereof, for use in a method of treating or lessening the severity in a subject of gut pain, wherein gut pain comprises inflammatory bowel disease pain, Crohn's disease pain or interstitial cystitis pain.

In another aspect, the invention features a compound of the invention, or a pharmaceutically acceptable salt or pharmaceutical composition thereof, for use in a method of treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In another aspect, the invention features a compound of the invention, or a pharmaceutically acceptable salt or pharmaceutical composition thereof, for use in a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion injury, complex regional pain syndrome, drug therapy induced neuralgia, cancer chemotherapy induced neuralgia, anti-retroviral therapy induced neuralgia, post spinal cord injury pain, small fiber neuropathy, idiopathic small-fiber neuropathy, idiopathic sensory neuropathy or trigeminal autonomic cephalalgia.

In another aspect, the invention features a compound of the invention, or a pharmaceutically acceptable salt or pharmaceutical composition thereof, for use in a method of treating or lessening the severity in a subject of musculoskeletal pain. In some aspects, the musculoskeletal pain comprises osteoarthritis pain.

In another aspect, the invention features a compound of the invention, or a pharmaceutically acceptable salt or pharmaceutical composition thereof, for use in a method of treating or lessening the severity in a subject of musculoskeletal pain, wherein musculoskeletal pain comprises osteoarthritis pain, back pain, cold pain, burn pain or dental pain.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain or vulvodynia.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises fibromyalgia pain.

In another aspect, the invention features a compound of the invention, or a pharmaceutically acceptable salt or pharmaceutical composition thereof, for use in a method of treating or lessening the severity in a subject of pathological cough.

In another aspect, the invention features a compound of the invention, or a pharmaceutically acceptable salt or pharmaceutical composition thereof, for use in a method of treating or lessening the severity in a subject of acute pain. In some aspects, the acute pain comprises acute post-operative pain.

In another aspect, the invention features a compound of the invention, or a pharmaceutically acceptable salt or pharmaceutical composition thereof, for use in a method of treating or lessening the severity in a subject of postsurgical pain (e.g., joint replacement pain, soft tissue surgery pain, herniorrhaphy pain, bunionectomy pain or abdominoplasty pain).

In another aspect, the invention features a compound of the invention, or a pharmaceutically acceptable salt or pharmaceutical composition thereof, for use in a method of treating or lessening the severity in a subject of bunionectomy pain.

In another aspect, the invention features a compound of the invention, or a pharmaceutically acceptable salt or pharmaceutical composition thereof, for use in a method of treating or lessening the severity in a subject of herniorrhaphy pain.

In another aspect, the invention features a compound of the invention, or a pharmaceutically acceptable salt or pharmaceutical composition thereof, for use in a method of treating or lessening the severity in a subject of abdominoplasty pain.

In another aspect, the invention features a compound of the invention, or a pharmaceutically acceptable salt or pharmaceutical composition thereof, for use in a method of treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for use in a method of treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In another aspect, the invention features a compound of the invention, or a pharmaceutically acceptable salt or pharmaceutical composition thereof, for use in a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a compound of the invention, or a pharmaceutically acceptable salt or pharmaceutical composition thereof, for use in a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Naᵥ1.8.

In another aspect, the invention features a compound of the invention, or a pharmaceutically acceptable salt or pharmaceutical composition thereof, for use in a method of treating or lessening the severity in a subject of acute pain, sub-acute and chronic pain, nociceptive pain, neuropathic pain, inflammatory pain, nociplastic pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention features a compound of the invention, or a pharmaceutically acceptable salt or pharmaceutical composition thereof, for use in a method of treating or lessening the severity in a subject of femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache, cluster headaches, chronic and acute neuropathic pain, post-herpetic neuralgia, diabetic neuropathy, HIV-associated neuropathy, trigeminal neuralgia, Charcot-Marie-Tooth neuropathy, hereditary sensory neuropathy, peripheral nerve injury, painful neuromas, ectopic proximal and distal discharges, radiculopathy, chemotherapy induced neuropathic pain, radiotherapy-induced neuropathic pain, persistent/chronic post-surgical pain (e.g., post amputation, post-thoracotomy, post-cardiac surgery), post-mastectomy pain, central pain, spinal cord injury pain, post-stroke pain, thalamic pain, phantom pain (e.g., following removal of lower extremity, upper extremity, breast), intractable pain, acute pain, acute post-operative pain, acute musculoskeletal pain, joint pain, mechanical low back pain, neck pain, tendonitis, injury pain, exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory pain, burn pain, trauma pain, acute intermittent pain, endometriosis, acute herpes zoster pain, sickle cell anemia, acute pancreatitis, breakthrough pain, orofacial pain, sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, urinary incontinence, pathological cough, hyperactive bladder, painful bladder syndrome, interstitial cystitis (IC), prostatitis, complex regional pain syndrome (CRPS), type I, complex regional pain syndrome (CRPS) type II, widespread pain, paroxysmal extreme pain, pruritus, tinnitus, or angina-induced pain.

In another aspect, the invention features a compound of the invention, or a pharmaceutically acceptable salt or pharmaceutical composition thereof, for use in a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbrosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Manufacture of Medicaments

In another aspect, the invention provides the use of a compound of the invention, or a pharmaceutically acceptable salt or pharmaceutical composition thereof, for the manufacture of a medicament.

In another aspect, the invention provides the use of a compound of the invention, a pharmaceutically acceptable salt thereof, or a pharmaceutical composition thereof for the manufacture of a medicament for use in inhibiting a voltage-gated sodium channel. In another aspect, the voltage-gated sodium channel is Naᵥ1.8.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In yet another aspect, the invention provides the use of the compound, pharmaceutically acceptable salt, or pharmaceutical composition described herein for the manufacture of a medicament for use in treating or lessening the severity in a subject of gut pain, wherein gut pain comprises inflammatory bowel disease pain, Crohn's disease pain or interstitial cystitis pain.

In yet another aspect, the invention provides a compound of the invention, a pharmaceutically acceptable salt thereof, or a pharmaceutical composition thereof for the manufacture of a medicament for use in treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy).

In yet another aspect, the invention provides the use of a compound of the invention, a pharmaceutically acceptable salt thereof, or a pharmaceutical composition thereof for the manufacture of a medicament for use in a treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma, traumatic neuroma, Morton's neuroma, nerve entrapment injury, spinal stenosis, carpal tunnel syndrome, radicular pain, sciatica pain, nerve avulsion injury, brachial plexus avulsion injury, complex regional pain syndrome, drug therapy induced neuralgia, cancer chemotherapy induced neuralgia, anti-retroviral therapy induced neuralgia, post spinal cord injury pain, small fiber neuropathy, idiopathic small-fiber neuropathy, idiopathic sensory neuropathy or trigeminal autonomic neuropathy.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain. In some aspects, the musculoskeletal pain comprises osteoarthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain, wherein musculoskeletal pain comprises osteoarthritis pain, back pain, cold pain, burn pain or dental pain.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain or vulvodynia.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises fibromyalgia pain.

In yet another aspect, the invention provides the use of a compound of the invention, a pharmaceutically acceptable salt thereof, or a pharmaceutical composition thereof for the manufacture of a medicament for use in treating or lessening the severity in a subject of pathological cough.

In yet another aspect, the invention provides the use of a compound of the invention, a pharmaceutically acceptable salt thereof, or a pharmaceutical composition thereof for the manufacture of a medicament for use in treating or lessening the severity in a subject of acute pain. In some aspects, the acute pain comprises acute post-operative pain.

In yet another aspect, the invention provides the use of a compound of the invention, a pharmaceutically acceptable salt thereof, or a pharmaceutical composition thereof for the manufacture of a medicament for use in treating or lessening the severity in a subject of postsurgical pain (e.g., joint replacement pain, soft tissue surgery pain, herniorrhaphy pain, bunionectomy pain or abdominoplasty pain).

In yet another aspect, the invention provides the use of a compound of the invention, a pharmaceutically acceptable salt thereof, or a pharmaceutical composition thereof for the manufacture of a medicament for use in treating or lessening the severity in a subject of herniorrhaphy pain.

In yet another aspect, the invention provides the use of a compound of the invention, a pharmaceutically acceptable salt thereof, or a pharmaceutical composition thereof for the manufacture of a medicament for use in treating or lessening the severity in a subject of bunionectomy pain.

In yet another aspect, the invention provides the use of a compound of the invention, a pharmaceutically acceptable salt thereof, or a pharmaceutical composition thereof for the manufacture of a medicament for use in treating or lessening the severity in a subject of abdominoplasty pain.

In yet another aspect, the invention provides the use of a compound of the invention, a pharmaceutically acceptable salt thereof, or a pharmaceutical composition thereof for the manufacture of a medicament for use in treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for the manufacture of a medicament for use in treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In yet another aspect, the invention provides the use of a compound of the invention, a pharmaceutically acceptable salt thereof, or a pharmaceutical composition thereof for the manufacture of a medicament for use in combination with one or more additional therapeutic agents administered concurrently with, prior to, or subsequent to treatment with the compound or pharmaceutical composition. In some embodiments, the additional therapeutic agent is a sodium channel inhibitor.

In another aspect, the invention provides the use of a compound of the invention, a pharmaceutically acceptable salt thereof, or a pharmaceutical composition thereof for the manufacture of a medicament for use in treating or lessening the severity of acute pain, sub-acute and chronic pain, nociceptive pain, neuropathic pain, inflammatory pain, nociplastic pain, arthritis, migraine, cluster headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention provides the use of a compound of the invention, a pharmaceutically acceptable salt thereof, or a pharmaceutical composition thereof for the manufacture of a medicament for use in treating or lessening the severity of femur cancer pain, non-malignant chronic bone pain, rheumatoid arthritis, osteoarthritis, spinal stenosis, neuropathic low back pain, myofascial pain syndrome, fibromyalgia, temporomandibular joint pain, chronic visceral pain, abdominal pain, pancreatic pain, IBS pain, chronic and acute headache pain, migraine, tension headache, cluster headaches, chronic and acute neuropathic pain, post-herpetic neuralgia, diabetic neuropathy, HIV-associated neuropathy, trigeminal neuralgia, Charcot-Marie-Tooth neuropathy, hereditary sensory neuropathy, peripheral nerve injury, painful neuromas, ectopic proximal and distal discharges, radiculopathy, chemotherapy induced neuropathic pain, radiotherapy-induced neuropathic pain, persistent/chronic post-surgical pain (e.g., post amputation, post-thoracotomy, post-cardiac surgery), post-mastectomy pain, central pain, spinal cord injury pain, post-stroke pain, thalamic pain, phantom pain (e.g., following removal of lower extremity, upper extremity, breast), intractable pain, acute pain, acute post-operative pain, acute musculoskeletal pain, joint pain, mechanical low back pain, neck pain, tendonitis, injury pain, exercise pain, acute visceral pain, pyelonephritis, appendicitis, cholecystitis, intestinal obstruction, hernias, chest pain, cardiac pain, pelvic pain, renal colic pain, acute obstetric pain, labor pain, cesarean section pain, acute inflammatory pain, burn pain, trauma pain, acute intermittent pain, endometriosis, acute herpes zoster pain, sickle cell anemia, acute pancreatitis, breakthrough pain, orofacial pain, sinusitis pain, dental pain, multiple sclerosis (MS) pain, pain in depression, leprosy pain, Behcet's disease pain, adiposis dolorosa, phlebitic pain, Guillain-Barre pain, painful legs and moving toes, Haglund syndrome, erythromelalgia pain, Fabry's disease pain, bladder and urogenital disease, urinary incontinence, pathological cough, hyperactive bladder, painful bladder syndrome, interstitial cystitis (IC), prostatitis, complex regional pain syndrome (CRPS), type I, complex regional pain syndrome (CRPS) type II, widespread pain, paroxysmal extreme pain, pruritus, tinnitus, or angina-induced pain.

In another aspect, the invention provides the use of a compound of the invention, a pharmaceutically acceptable salt thereof, or a pharmaceutical composition thereof for the manufacture of a medicament for use in treating or lessening the severity of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbrosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Administration of Compounds, Pharmaceutically Acceptable Salts, and Compositions

In certain embodiments of the invention, an “effective amount” of a compound of the invention, a pharmaceutically acceptable salt thereof, or a pharmaceutical composition thereof is that amount effective for treating or lessening the severity of one or more of the conditions recited above.

The compounds, salts, and compositions, according to the method of the invention, may be administered using any amount and any route of administration effective for treating or lessening the severity of one or more of the pain or non-pain diseases recited herein. The exact amount required will vary from subject to subject, depending on the species, age, and general condition of the subject, the severity of the condition, the particular agent, its mode of administration, and the like. The compounds, salts, and compositions of the invention are preferably formulated in dosage unit form for ease of administration and uniformity of dosage. The expression “dosage unit form” as used herein refers to a physically discrete unit of agent appropriate for the subject to be treated. It will be understood, however, that the total daily usage of the compounds, salts, and compositions of the invention will be decided by the attending physician within the scope of sound medical judgment. The specific effective dose level for any particular subject or organism will depend upon a variety of factors including the disorder being treated and the severity of the disorder, the activity of the specific compound or salt employed, the specific composition employed, the age, body weight, general health, sex and diet of the subject, the time of administration, route of administration, and rate of excretion of the specific compound or salt employed, the duration of the treatment, drugs used in combination or coincidental with the specific compound or salt employed, and like factors well known in the medical arts. The term “subject” or “patient,” as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the condition being treated. In certain embodiments, the compound, salts, and compositions of the invention may be administered orally or parenterally at dosage levels of about 0.001 mg/kg to about 1000 mg/kg, one or more times a day, effective to obtain the desired therapeutic effect.

Liquid dosage forms for oral administration include, but are not limited to, pharmaceutically acceptable emulsions, microemulsions, solutions, suspensions, syrups and elixirs. In addition to the active compound or salt, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of the compounds of the invention, it is often desirable to slow the absorption of the compounds from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound or salt of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound or salt is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compound or salt can also be in microencapsulated form with one or more excipients as noted above. The solid dosage forms of tablets, dragees, capsules, pills, and granules can be prepared with coatings and shells such as enteric coatings, release-controlling coatings and other coatings well known in the pharmaceutical formulating art. In such solid dosage forms, the active compound or salt may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound or salt of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of voltage-gated sodium channels. In one embodiment, the compounds are inhibitors of Naᵥ1.8 and thus, without wishing to be bound by any particular theory, the compounds, salts, and compositions are particularly useful for treating or lessening the severity of a disease, condition, or disorder where activation or hyperactivity of Naᵥ1.8 is implicated in the disease, condition, or disorder. When activation or hyperactivity of Naᵥ1.8 is implicated in a particular disease, condition, or disorder, the disease, condition, or disorder may also be referred to as a “Naᵥ1.8-mediated disease, condition or disorder.” Accordingly, in another aspect, the invention provides a method for treating or lessening the severity of a disease, condition, or disorder where activation or hyperactivity of Naᵥ1.8 is implicated in the disease state.

The activity of a compound utilized in this invention as an inhibitor of Naᵥ1.8 may be assayed according to methods described generally in International Publication No. WO 2014/120808 A9 and U.S. Publication No. 2014/0213616 A1, both of which are incorporated by reference in their entirety, methods described herein, and other methods known and available to one of ordinary skill in the art.

### Additional Therapeutic Agents

It will also be appreciated that the compounds, salts, and pharmaceutically acceptable compositions of the invention can be employed in combination therapies, that is, the compounds, salts, and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.” For example, exemplary additional therapeutic agents include, but are not limited to: non-opioid analgesics (indoles such as Etodolac, Indomethacin, Sulindac, Tolmetin, naphthylalkanones such as Nabumetone, oxicams such as Piroxicam, para-aminophenol derivatives, such as Acetaminophen, propionic acids such as Fenoprofen, Flurbiprofen, Ibuprofen, Ketoprofen, Naproxen, Naproxen sodium, Oxaprozin, salicylates such as Aspirin, Choline magnesium trisalicylate, Diflunisal, fenamates such as meclofenamic acid, Mefenamic acid, and pyrazoles such as Phenylbutazone), or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blockade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Nineteenth Edition, Ed. Robert S. Porter and Justin L. Kaplan, Merck Sharp & Dohme Corp., a subsidiary of Merck & Co., Inc., 2011, and the Food and Drug Administration website, www.fda.gov, the entire contents of which are hereby incorporated by reference.

In another embodiment, additional appropriate therapeutic agents are selected from the following:
(1) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine, pentazocine, or difelikefalin;
(2) a nonsteroidal antiinflammatory drug (NSAID), e.g. aspirin, diclofenac, diflunisal, etodolac, fenbufen, fenoprofen, flufenisal, flurbiprofen, ibuprofen (including without limitation intravenous ibuprofen (e.g., Caldolor®)), indomethacin, ketoprofen, ketorolac (including without limitation ketorolac tromethamine (e.g., Toradol®)), meclofenamic acid, mefenamic acid, meloxicam, IV meloxicam (e.g., Anjeso®), nabumetone, naproxen, nimesulide, nitroflurbiprofen, olsalazine, oxaprozin, phenylbutazone, piroxicam, sulfasalazine, sulindac, tolmetin or zomepirac;
(3) a barbiturate sedative, e.g. amobarbital, aprobarbital, butabarbital, butalbital, mephobarbital, metharbital, methohexital, pentobarbital, phenobarbital, secobarbital, talbutal, thiamylal or thiopental;
(4) a benzodiazepine having a sedative action, e.g. chlordiazepoxide, clorazepate, diazepam, flurazepam, lorazepam, oxazepam, temazepam or triazolam;
(5) a histamine (H₁) antagonist having a sedative action, e.g. diphenhydramine, pyrilamine, promethazine, chlorpheniramine or chlorcyclizine;
(6) a sedative such as glutethimide, meprobamate, methaqualone or dichloralphenazone;
(7) a skeletal muscle relaxant, e.g. baclofen, carisoprodol, chlorzoxazone, cyclobenzaprine, methocarbamol or orphenadrine;
(8) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
(9) an alpha-adrenergic, e.g. doxazosin, tamsulosin, clonidine, guanfacine, dexmedetomidine, modafinil, or 4-amino-6,7-dimethoxy-2-(5-methane-sulfonamido-1,2,3,4-tetrahydroisoquinolin-2-yl)-5-(2-pyridyl) quinazoline;
(10) a tricyclic antidepressant, e.g. desipramine, imipramine, amitriptyline or nortriptyline;
(11) an anticonvulsant, e.g. carbamazepine (Tegretol®), lamotrigine, topiramate, lacosamide (Vimpat®) or valproate;
(12) a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
(13) a muscarinic antagonist, e.g oxybutynin, tolterodine, propiverine, tropsium chloride, darifenacin, solifenacin, temiverine and ipratropium;
(14) a COX-2 selective inhibitor, e.g. celecoxib, rofecoxib, parecoxib, valdecoxib, deracoxib, etoricoxib, or lumiracoxib;
(15) a coal-tar analgesic, in particular paracetamol;
(16) a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
(17) a vanilloid receptor agonist (e.g. resinferatoxin or civamide) or antagonist (e.g. capsazepine, GRC-15300);
(18) a beta-adrenergic such as propranolol;
(19) a local anesthetic such as mexiletine;
(20) a corticosteroid such as dexamethasone;
(21) a 5-HT receptor agonist or antagonist, particularly a 5-HT1B/1D agonist such as eletriptan, sumatriptan, naratriptan, zolmitriptan or rizatriptan;
(22) a 5-HT2A receptor antagonist such as R (+)-alpha-(2,3-dimethoxy-phenyl)-1-[2-(4-fluorophenylethyl)]-4-piperidinemethanol (MDL-100907);
(23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
(24) Tramadol®, Tramadol ER (Ultram ER®), IV Tramadol, Tapentadol ER (Nucynta®);
(25) a PDE5 inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl) pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
(26) an alpha-2-delta ligand such as gabapentin (Neurontin®), gabapentin GR (Gralise®), gabapentin, enacarbil (Horizant®), pregabalin (Lyrica®), 3-methyl gabapentin, (1[alpha],3 [alpha],5 [alpha]) (3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy) proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
(27) a cannabinoid such as KHK-6188;
(28) metabotropic glutamate subtype 1 receptor (mGluRI) antagonist;
(29) a serotonin reuptake inhibitor such as sertraline, sertraline metabolite demethylsertraline,
fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
(30) a noradrenaline (norepinephrine) reuptake inhibitor, such as maprotiline, lofepramine, mirtazepine, oxaprotiline, fezolamine, tomoxetine, mianserin, bupropion, bupropion metabolite hydroxybupropion, nomifensine and viloxazine (Vivalan®), especially a selective noradrenaline reuptake inhibitor such as reboxetine, in particular (S,S)-reboxetine;
(31) a dual serotonin-noradrenaline reuptake inhibitor, such as venlafaxine, venlafaxine metabolite O-desmethylvenlafaxine, clomipramine, clomipramine metabolite desmethylclomipramine, duloxetine (Cymbalta®), milnacipran and imipramine;
(32) an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-S-chloro-S-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3-pyridinecarbonitrile, 2-[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, NXN-462, or guanidinoethyldisulfide;
(33) an acetylcholinesterase inhibitor such as donepezil;
(34) a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(15)-1-({[5-chloro-2-(3-fluorophenoxy)pyridin-3-yl]carbonyl}amino)ethyl]benzoic acid;
(35) a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870;
(36) a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl)-1,4-benzoquinone (CV-6504);
(37) a sodium channel blocker, such as lidocaine, lidocaine plus tetracaine cream (ZRS-201) or eslicarbazepine acetate;
(38) a Naᵥ1.7 blocker, such as XEN-402, XEN403, TV-45070, PF-05089771, CNV1014802, GDC-0276, RG7893 BIIB-074 (Vixotrigine), BIIB-095, ASP-1807, DSP-3905, OLP-1002, RQ-00432979, FX-301, DWP-1706, DWP-17061, IMB-110, IMB-111, IMB-112 and such as those disclosed in WO2011/140425 (US2011/306607), WO2012/106499 (US2012196869), WO2012/112743 (US2012245136), WO2012/125613 (US2012264749), WO2012/116440 (US2014187533), WO2011026240 (US2012220605), U.S. Pat. Nos. 8,883,840, 8,466,188, WO2013/109521 (US2015005304), CN111217776, or WO2020/117626, the entire contents of each application hereby incorporated by reference;
(38a) a Naᵥ1.7 blocker such as (2-benzylspiro[3,4-dihydropyrrolo[1,2-a]pyrazine-1,4′-piperidine]-1′-yl)-(4-isopropoxy-3-methyl-phenyl) methanone, 2,2,2-trifluoro-1-[1′-[3-methoxy-4-[2-(trifluoromethoxy)ethoxy]benzoyl]-2,4-dimethyl-spiro[3,4-dihydropyrrolo[1,2-a]pyrazine-1,4′-piperidine]-6-ylJethanone, [8-fluoro-2-methyl-6-(trifluoromethyl)spiro[3,4-dihydropyrrolo[1,2-a]pyrazine-1,4′-piperidine]-1′-yl]-(4-isobutoxy-3-methoxy-phenyl) methanone, 1-(4-benzhydrylpiperazin-1-yl)-3-[2-(3,4-dimethylphenoxy)ethoxy]propan-2-ol, (4-butoxy-3-methoxy-phenyl)-[2-methyl-6-(trifluoromethyl)spiro[3,4-dihydropyrrolo[1,2-a]pyrazine-1,4′-piperidine]-1′-yl]methanone, [8-fluoro-2-methyl-6-(trifluoromethyl)spiro[3,4-dihydropyrrolo[1,2-a]pyrazine-1,4′-piperidine]-1′-yl]-(5-isopropoxy-6-methyl-2-pyridyl) methanone, (4-isopropoxy-3-methyl-phenyl)-[2-methyl-6-(1,1,2,2,2-pentafluoroethyl)spiro[3,4-dihydropyrrolo[1,2-a]pyrazine-1,4′-piperidine]-1′-yl]methanone, 5-[2-methyl-4-[2-methyl-6-(2,2,2-trifluoroacetyl)spiro[3,4-dihydropyrrolo[1,2-a]pyrazine-1,4′-piperidine]-1′-carbonyl]phenyl]pyridine-2-carbonitrile, (4-isopropoxy-3-methyl-phenyl)-[6-(trifluoromethyl)spiro[3,4-dihydro-2H-pyrrolo[1,2-a]pyrazine-1,4′-piperidine]-1′-yl]methanone, 2,2,2-trifluoro-1-[1′-[3-methoxy-4-[2-(trifluoromethoxy)ethoxy]benzoyl]-2-methyl-spiro[3,4-dihydropyrrolo[1,2-a]pyrazine-1,4′-piperidine]-6-yl]ethanone, 2,2,2-trifluoro-1-[1′-(5-isopropoxy-6-methyl-pyridine-2-carbonyl)-3,3-dimethyl-spiro[2,4-dihydropyrrolo[1,2-a]pyrazine-1,4′-piperidine]-6-yl]ethanone, 2,2,2-trifluoro-1-[1′-(5-isopentyloxypyridine-2-carbonyl)-2-methyl-spiro[3,4-dihydropyrrolo[1,2-a]pyrazine-1,4′-piperidine]-6-yl]ethanone, (4-isopropoxy-3-methoxy-phenyl)-[2-methyl-6-(trifluoromethyl)spiro[3,4-dihydropyrrolo[1,2-a]pyrazine-1,4′-piperidine]-1′-yl]methanone, 2,2,2-trifluoro-1-[1′-(5-isopentyloxypyridine-2-carbonyl)-2,4-dimethyl-spiro[3,4-dihydropyrrolo[1,2-a]pyrazine-1,4′-piperidine]-6-yl]ethanone, 1-[(3S)-2,3-dimethyl-1′-[4-(3,3,3-trifluoropropoxymethyl)benzoyl]spiro[3,4-dihydropyrrolo[1,2-a]pyrazine-1,4′-piperidine]-6-yl]-2,2,2-trifluoro-ethanone, [8-fluoro-2-methyl-6-(trifluoromethyl)spiro[3,4-dihydropyrrolo[1,2-a]pyrazine-1,4′-piperidine]-1′-yl]-[3-methoxy-4-[(1R)-1-methylpropoxy]phenyl]methanone, 2,2,2-trifluoro-1-[1′-(5-isopropoxy-6-methyl-pyridine-2-carbonyl)-2,4-dimethyl-spiro[3,4-dihydropyrrolo[1,2-a]pyrazine-1,4′-piperidine]-6-yl]ethanone, 1-[1′-[4-methoxy-3-(trifluoromethyl)benzoyl]-2-methyl-spiro[3,4-dihydropyrrolo[1,2-a]pyrazine-1,4′-piperidine]-6-yl]-2,2-dimethyl-propan-1-one, (4-isopropoxy-3-methyl-phenyl)-[2-methyl-6-(trifluoromethyl)spiro[3,4-dihydropyrrolo[1,2-a]pyrazine-1,4′-piperidine]-1′-yl]methanone, [2-methyl-6-(1-methylcyclopropanecarbonyl)spiro[3,4-dihydropyrrolo[1,2-a]pyrazine-1,4′-piperidine]-1′-yl]-[4-(3,3,3-trifluoropropoxymethyl)phenyl]methanone, 4-bromo-N-(4-bromophenyl)-3-[(1-methyl-2-oxo-4-piperidyl)sulfamoyl]benzamide or (3-chloro-4-isopropoxy-phenyl)-[2-methyl-6-(1,1,2,2,2-pentafluoroethyl)spiro[3,4-dihydropyrrolo[1,2-a]pyrazine-1,4′-piperidine]-1′-yl]methanone.
(39) a Naᵥ1.8 blocker, such as PF-04531083, PF-06372865 and such as those disclosed in WO2008/135826 (US2009048306), WO2006/011050 (US2008312235), WO2013/061205 (US2014296313), US20130303535, WO2013131018, U.S. Pat. No. 8,466,188, WO2013114250 (US2013274243), WO2014/120808 (US2014213616), WO2014/120815 (US2014228371) WO2014/120820 (US2014221435), WO2015/010065 (US20160152561), WO2015/089361 (US20150166589), WO2019/014352 (US20190016671), WO2018/213426, WO2020/146682, WO2020/146612, WO2020/014243, WO2020/014246, WO2020/092187, WO2020/092667 (US2020140411), WO2020/261114, WO2020/140959, WO2020/151728, WO2021/032074, WO2021/047622 (CN112479996), CN112390745, CN111808019, CN112225695, CN112457294, CN112300051, CN112300069, and CN112441969, the entire contents of each application hereby incorporated by reference;
(39a) a Naᵥ1.8 blocker such as 4,5-dichloro-2-(4-fluoro-2-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-4-(perfluoroethyl)benzamide, 4,5-dichloro-2-(4-fluorophenoxy)-N-(2-oxo-1,2-dihydropyridin-4-yl)benzamide, 4,5-dichloro-2-(3-fluoro-4-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-5-(trifluoromethyl)benzamide, N-(2-oxo-1,2-dihydropyridin-4-yl)-2-(4-(trifluoromethoxy)phenoxy)-4-(trifluoromethyl)benzamide, 2-(4-fluorophenoxy)-N-(2-oxo-1,2-dihydropyridin-4-yl)-4-(perfluoroethyl)benzamide, 5-chloro-2-(4-fluoro-2-methoxyphenoxy)-N-(2-oxo-1,2-dihydropyridin-4-yl)benzamide, N-(2-oxo-1,2-dihydropyridin-4-yl)-2-(4-(trifluoromethoxy)phenoxy)-5-(trifluoromethyl)benzamide, 2-(4-fluoro-2-methylphenoxy)-N-(2-oxo-1,2-dihydropyridin-4-yl)-5-(trifluoromethyl)benzamide, 2-(2-chloro-4-fluorophenoxy)-N-(2-oxo-1,2-dihydropyridin-4-yl)-5-(trifluoromethyl)benzamide, 5-chloro-2-(4-fluoro-2-methylphenoxy)-N-(2-oxo-1,2-dihydropyridin-4-yl)benzamide, 4-chloro-2-(4-fluoro-2-methylphenoxy)-N-(2-oxo-1,2-dihydropyridin-4-yl)benzamide, 5-chloro-2-(2-chloro-4-fluorophenoxy)-N-(2-oxo-1,2-dihydropyridin-4-yl)benzamide, 2-((5-fluoro-2-hydroxybenzyl)oxy)-N-(2-oxo-1,2-dihydropyridin-4-yl)-4-(trifluoromethyl)benzamide, N-(2-oxo-1,2-dihydropyridin-4-yl)-2-(o-tolyloxy)-5-(trifluoromethyl)benzamide, 2-(2,4-difluorophenoxy)-N-(2-oxo-1,2-dihydropyridin-4-yl)-4-(trifluoromethyl)benzamide, N-(2-oxo-1,2-dihydropyridin-4-yl)-2-(2-(trifluoromethoxy)phenoxy)-5-(trifluoromethyl)benzamide, 2-(4-fluorophenoxy)-N-(2-oxo-1,2-dihydropyridin-4-yl)-5-(trifluoromethyl)benzamide, 2-(4-fluoro-2-methyl-phenoxy)-N-(2-oxo-1H-pyridin-4-yl)-4-(trifluoromethyl)benzamide, [4-[[2-(4-fluoro-2-methyl-phenoxy)-4-(trifluoromethyl)benzoyl]amino]-2-oxo-1-pyridyl]methyl dihydrogen phosphate, 2-(4-fluoro-2-(methyl-d₃)phenoxy)-N-(2-oxo-1,2-dihydropyridin-4-yl)-4-(trifluoromethyl)benzamide, (4-(2-(4-fluoro-2-(methyl-d₃)phenoxy)-4-(trifluoromethyl)benzamido)-2-oxopyridin-1(2H)-yl)methyl dihydrogen phosphate, 3-(4-fluoro-2-methoxyphenoxy)-N-(3-(methylsulfonyl)phenyl)quinoxaline-2-carboxamide, 3-(2-chloro-4-fluorophenoxy)-N-(3-sulfamoylphenyl)quinoxaline-2-carboxamide, 3-(2-chloro-4-methoxyphenoxy)-N-(3-sulfamoylphenyl)quinoxaline-2-carboxamide, 3-(4-chloro-2-methoxyphenoxy)-N-(3-sulfamoylphenyl)quinoxaline-2-carboxamide, 4-(3-(4-(trifluoromethoxy)phenoxy)quinoxaline-2-carboxamido)picolinic acid, 2-(2,4-difluorophenoxy)-N-(3-sulfamoylphenyl) quinoline-3-carboxamide, 2-(4-fluoro-2-methoxyphenoxy)-N-(3-sulfamoylphenyl) quinoline-3-carboxamide, 3-(2,4-difluorophenoxy)-N-(3-sulfamoylphenyl)quinoxaline-2-carboxamide, N-(3-sulfamoylphenyl)-2-(4-(trifluoromethoxy)phenoxy) quinoline-3-carboxamide, N-(3-sulfamoylphenyl)-3-(4-(trifluoromethoxy)phenoxy)quinoxaline-2-carboxamide, 3-(4-chloro-2-methylphenoxy)-N-(3-sulfamoylphenyl)quinoxaline-2-carboxamide, 5-(3-(4-(trifluoromethoxy)phenoxy)quinoxaline-2-carboxamido)picolinic acid, 3-(4-fluoro-2-methoxyphenoxy)-N-(2-oxo-2,3-dihydro-1H-benzo[d]imidazol-5-yl)quinoxaline-2-carboxamide, 3-(4-fluoro-2-methoxyphenoxy)-N-(pyridin-4-yl)quinoxaline-2-carboxamide, 3-(4-fluorophenoxy)-N-(3-sulfamoylphenyl)quinoxaline-2-carboxamide, N-(3-cyanophenyl)-3-(4-fluoro-2-methoxyphenoxy)quinoxaline-2-carboxamide, N-(4-carbamoylphenyl)-3-(4-fluoro-2-methoxyphenoxy)quinoxaline-2-carboxamide, 4-(3-(4-(trifluoromethoxy)phenoxy)quinoxaline-2-carboxamido)benzoic acid, N-(4-cyanophenyl)-3-(4-fluoro-2-methoxyphenoxy)quinoxaline-2-carboxamide, 5-(4,5-dichloro-2-(4-fluoro-2-methoxyphenoxy)benzamido)picolinic acid, 5-(2-(2,4-dimethoxyphenoxy)-4,6-bis(trifluoromethyl)benzamido)picolinic acid, 4-(4,5-dichloro-2-(4-fluoro-2-methoxyphenoxy)benzamido)benzoic acid, 5-(2-(4-fluoro-2-methoxyphenoxy)-4,6-bis(trifluoromethyl)benzamido)picolinic acid, 4-(2-(4-fluoro-2-methoxyphenoxy)-4-(perfluoroethyl)benzamido)benzoic acid, 5-(2-(4-fluoro-2-methoxyphenoxy)-4-(perfluoroethyl)benzamido)picolinic acid, 4-(2-(4-fluoro-2-methylphenoxy)-4-(trifluoromethyl)benzamido)benzoic acid, 5-(4,5-dichloro-2-(4-fluoro-2-methoxyphenoxy)benzamido)picolinic acid, 4-(2-(2-chloro-4-fluorophenoxy)-4-(perfluoroethyl)benzamido)benzoic acid, 4-(2-(4-fluoro-2-methylphenoxy)-4-(perfluoroethyl)benzamido)benzoic acid, 4-(4,5-dichloro-2-(4-(trifluoromethoxy)phenoxy)benzamido)benzoic acid, 4-(4,5-dichloro-2-(4-chloro-2-methylphenoxy)benzamido)benzoic acid, 5-(4-(tert-butyl)-2-(4-fluoro-2-methoxyphenoxy)benzamido)picolinic acid, 5-(4,5-dichloro-2-(4-(trifluoromethoxy)phenoxy)benzamido)picolinic acid, 4-(4,5-dichloro-2-(4-fluoro-2-methylphenoxy)benzamido)benzoic acid, 5-(4,5-dichloro-2-(2,4-dimethoxyphenoxy)benzamido)picolinic acid, 5-(4,5-dichloro-2-(2-chloro-4-fluorophenoxy)benzamido)picolinic acid, 5-(4,5-dichloro-2-(4-fluoro-2-methylphenoxy)benzamido)picolinic acid, 4-(4,5-dichloro-2-(4-chloro-2-methoxyphenoxy)benzamido)benzoic acid, 5-(4,5-dichloro-2-(2,4-difluorophenoxy)benzamido)picolinic acid, 2-(4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-6-(trifluoromethyl)benzamide, 2-(2-chloro-4-fluorophenoxy)-5-(difluoromethyl)-N-(3-sulfamoylphenyl)benzamide, 2-(4-fluorophenoxy)-4-(perfluoroethyl)-N-(3-sulfamoylphenyl)benzamide, 2-(4-chloro-2-methoxyphenoxy)-4-(perfluoroethyl)-N-(3-sulfamoylphenyl)benzamide, 2-(4-fluoro-2-methoxyphenoxy)-N-(3-sulfamoylphenyl)-5-(trifluoromethyl)benzamide, 5-chloro-2-(4-fluoro-2-methylphenoxy)-N-(3-sulfamoylphenyl)benzamide, 4,5-dichloro-2-(4-fluoro-2-methoxyphenoxy)-N-(3-sulfamoylphenyl)benzamide, 2,4-dichloro-6-(4-chloro-2-methoxyphenoxy)-N-(3-sulfamoylphenyl)benzamide, 2,4-dichloro-6-(4-fluoro-2-methylphenoxy)-N-(3-sulfamoylphenyl)benzamide, 2-(4-fluoro-2-methoxyphenoxy)-N-(3-sulfamoylphenyl)-4,6-bis(trifluoromethyl)benzamide, 2-(4-fluoro-2-methylphenoxy)-N-(3-sulfamoylphenyl)-4,6-bis(trifluoromethyl)benzamide, 5-chloro-2-(2-chloro-4-fluorophenoxy)-N-(3-sulfamoylphenyl)benzamide, 2-(4-fluoro-2-methoxyphenoxy)-N-(3-sulfamoylphenyl)-4-(trifluoromethoxy)benzamide, 2-(4-fluoro-2-methoxyphenoxy)-N-(3-sulfamoylphenyl)-4-(trifluoromethyl)benzamide, 4,5-dichloro-2-(4-fluorophenoxy)-N-(3-sulfamoylphenyl)benzamide, 2-(4-fluoro-2-methoxyphenoxy)-4-(perfluoroethyl)-N-(3-sulfamoylphenyl)benzamide, 5-fluoro-2-(4-fluoro-2-methylphenoxy)-N-(3-sulfamoylphenyl)benzamide, 2-(2-chloro-4-fluorophenoxy)-4-cyano-N-(3-sulfamoylphenyl)benzamide, N-(3-sulfamoylphenyl)-2-(4-(trifluoromethoxy)phenoxy)-4-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethyl)benzamide, N-(3-carbamoyl-4-fluoro-phenyl)-2-fluoro-6-[2-methoxy-4-(trifluoromethoxy)phenoxy]-3-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethoxy)benzamide, 4-[[2-fluoro-6-[2-methoxy-4-(trifluoromethoxy)phenoxy]-3-(trifluoromethyl)benzoyl]amino]pyridine-2-carboxamide, 4-[[3-chloro-2-fluoro-6-[2-methoxy-4-(trifluoromethoxy)phenoxy]benzoyl]amino]pyridine-2-carboxamide, 4-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-3-(difluoromethyl)-2-fluoro-6-[2-methoxy-4-(trifluoromethoxy)phenoxy]benzamide, 4-[2-fluoro-6-[2-(trideuteriomethoxy)-4-(trifluoromethoxy)phenoxy]-3-(trifluoromethoxy)benzoyl]amino]pyridine-2-carboxamide, N-(3-carbamoyl-4-fluoro-phenyl)-6-[2-chloro-4-(trifluoromethoxy)phenoxy]-2-fluoro-3-(trifluoromethyl)benzamide, N-(3-carbamoyl-4-fluoro-phenyl)-2-fluoro-6-[2-methyl-4-(trifluoromethoxy)phenoxy]-3-(trifluoromethyl)benzamide, N-(3-carbamoyl-4-fluoro-phenyl)-2,3,4-trifluoro-6-[2-methoxy-4-(trifluoromethoxy)phenoxy]benzamide, N-(2-carbamoyl-4-pyridyl)-3-fluoro-5-[2-methoxy-4-(trifluoromethoxy)phenoxy]-2-(trifluoromethyl)pyridine-4-carboxamide, 4-[6-[2-(difluoromethoxy)-4-(trifluoromethoxy)phenoxy]-2-fluoro-3-(trifluoromethyl)benzoyl]amino]pyridine-2-carboxamide, N-(3-carbamoyl-4-fluoro-phenyl)-6-[3-chloro-4-(trifluoromethoxy)phenoxy]-2-fluoro-3-(trifluoromethyl)benzamide, N-(3-carbamoyl-4-fluoro-phenyl)-2-fluoro-6-[4-(trifluoromethoxy)phenoxy]-3-(trifluoromethyl)benzamide, N-(4-carbamoyl-3-fluoro-phenyl)-2-fluoro-6-[2-methoxy-4-(trifluoromethoxy)phenoxy]-3-(trifluoromethyl)benzamide, 4-[2-fluoro-6-[2-(trideuteriomethoxy)-4-(trifluoromethoxy)phenoxy]-4-(trifluoromethyl)benzoyl]amino]pyridine-2-carboxamide, N-(3-carbamoyl-4-fluoro-phenyl)-2-fluoro-6-[3-fluoro-4-(trifluoromethoxy)phenoxy]-3-(trifluoromethyl)benzamide, N-(3-carbamoyl-4-fluoro-phenyl)-2-[2-methoxy-4-(trifluoromethoxy)phenoxy]-5-(1,1,2,2,2-pentafluoroethyl)benzamide, 4-[4-(difluoromethoxy)-2-fluoro-6-[2-methoxy-4-(trifluoromethoxy)phenoxy]benzoyl]amino]pyridine-2-carboxamide, N-(3-carbamoyl-4-fluoro-phenyl)-2-fluoro-6-[2-fluoro-4-(trifluoromethoxy)phenoxy]-3-(trifluoromethyl)benzamide, 4-[4-cyclopropyl-2-fluoro-6-[2-methoxy-4-(trifluoromethoxy)phenoxy]benzoyl]amino]pyridine-2-carboxamide, N-(3-carbamoyl-4-fluoro-phenyl)-5-fluoro-2-[2-methoxy-4-(trifluoromethoxy)phenoxy]-4-(trifluoromethyl)benzamide, 5-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-2-fluoro-6-(4-fluorophenoxy)-3-(trifluoromethyl)benzamide, 4-(2-fluoro-6-(2-methoxy-4-(trifluoromethoxy)phenoxy)-3-(trifluoromethyl)benzamido) picolinamide, or 4-[2-fluoro-6-[3-fluoro-2-methoxy-4-(trifluoromethoxy)phenoxy]-3-(trifluoromethyl)benzoyl]amino]pyridine-2-carboxamide;
(40) a combined Naᵥ1.7 and Naᵥ1.8 blocker, such as DSP-2230, Lohocla201 or BL-1021;
(41) a 5-HT3 antagonist, such as ondansetron;
(42) a TPRV 1 receptor agonist, such as capsaicin (NeurogesX®, Qutenza®), and the pharmaceutically acceptable salts and solvates thereof;
(43) a nicotinic receptor antagonist, such as varenicline;
(44) an N-type calcium channel antagonist, such as Z-160,
(45) a nerve growth factor antagonist, such as tanezumab;
(46) an endopeptidase stimulant, such as senrebotase;
(47) an angiotensin II antagonist, such as EMA-401;
(48) acetaminophen (including without limitation intravenous acetaminophen (e.g., Ofirmev®));
(49) bupivacaine (including without limitation bupivacaine liposome injectable suspension (e.g., Exparel®) bupivacaine ER (Posimir), bupivacaine collagen (Xaracoll) and transdermal bupivacaine (Eladur®)); and
(50) bupivacaine and meloxicam combination (e.g., HTX-011).

In one embodiment, the additional appropriate therapeutic agents are selected from V-116517, Pregabalin, controlled release Pregabalin, Ezogabine (Potiga®). Ketamine/amitriptyline topical cream (Amiket®), AVP-923, Perampanel (E-2007), Ralfinamide, transdermal bupivacaine (Eladur®), CNV1014802, JNJ-10234094 (Carisbamate), BMS-954561 or ARC-4558.

In another embodiment, the additional appropriate therapeutic agents are selected from N-(6-amino-5-(2,3,5-trichlorophenyl)pyridin-2-yl) acetamide, N-(6-amino-5-(2-chloro-5-methoxyphenyl)pyridin-2-yl)-1-methyl-1H-pyrazole-5-carboxamide, or 3-((4-(4-(trifluoromethoxy)phenyl)-1H-imidazol-2-yl)methyl) oxetan-3-amine.

In another embodiment, the additional therapeutic agent is selected from a GlyT2/5HT2 inhibitor, such as Operanserin (VVZ149), a TRPV modulator such as CA008, CMX-020, NEO6860, FTABS, CNTX4975, MCP101, MDR16523, or MDR652, a EGR1 inhibitor such as Brivoglide (AYX1), an NGF inhibitor such as Tanezumab, Fasinumab, ASP6294, MEDI7352, a Mu opioid agonist such as Cebranopadol, NKTR181 (oxycodegol), a CB-1 agonist such as NEO1940 (AZN1940), an imidazoline 12 agonist such as CR4056 or a p75NTR-Fc modulator such as LEVI-04.

In another embodiment, the additional therapeutic agent is oliceridine or ropivacaine (TLC590).

In another embodiment, the additional therapeutic agent is a Naᵥ1.7 blocker such as ST-2427 or ST-2578 and those disclosed in WO2010129864, WO2015157559, WO2017059385, WO2018183781, WO2018183782, WO2020072835, and WO2022036297 the entire contents of each application hereby incorporated by reference. In some embodiments, the additional therapeutic agent is a Naᵥ1.7 blocker disclosed in WO2020072835. In some embodiments, the additional therapeutic agent is a Naᵥ1.7 blocker disclosed in WO2022036297.

In another embodiment, the additional therapeutic agent is ASP18071, CC-8464, ANP-230, ANP-231, NOC-100, NTX-1175, ASN008, NW3509, AM-6120, AM-8145, AM-0422, BL-017881, NTM-006, Opiranserin (Unafra™), brivoligide, SR419, NRD.E1, LX9211, LY3016859, ISC-17536, NFX-88, LAT-8881, AP-235, NYX 2925, CNTX-6016, S-600918, S-637880, RQ-00434739, KLS-2031, MEDI 7352, or XT-150.

In another embodiment, the additional therapeutic agent is Olinvyk, Zynrelef, Seglentis, Neumentum, Nevakar, HTX-034, CPL-01, ACP-044, HRS-4800, Tarlige, BAY2395840, LY3526318, Eliapixant, TRV045, RTA901, NRD1355-E1, MT-8554, LY3556050, AP-325, tetrodotoxin, Otenaproxesul, CFTX-1554, Funapide, iN1011-N17, JMKX000623, ETX-801, or ACD440.

In another embodiment, the additional therapeutic agent is a compound disclosed in WO2021257490, WO2021257420, WO2021257418, WO2020014246, WO2020092187, WO2020092667, WO2020261114, CN112457294, CN112225695, CN111808019, WO2021032074, WO2020151728, WO2020140959, WO2022037641, WO2022037647, CN112300051, CN112300069, WO2014120808, WO2015089361, WO2019014352, WO2021113627, WO2013086229, WO2013134518, WO2014211173, WO2014201206, WO2016141035, WO2021252818, WO2021252822, and WO2021252820.

In some embodiments, the additional therapeutic agent is a compound disclosed in WO2013086229. In some embodiments, the additional therapeutic agent is a compound disclosed in WO2013134518. In some embodiments, the additional therapeutic agent is a compound disclosed in WO2014211173. In some embodiments, the additional therapeutic agent is a compound disclosed in WO2014201206. In some embodiments, the additional therapeutic agent is a compound disclosed in WO2016141035. In some embodiments, the additional therapeutic agent is a compound disclosed in WO2021252818. In some embodiments, the additional therapeutic agent is a compound disclosed in WO2021252822. In some embodiments, the additional therapeutic agent is a compound disclosed in WO2021252820. In some embodiments, the additional therapeutic agent is a compound disclosed in WO2020072835. In some embodiments, the additional therapeutic agent is a compound disclosed in WO2022036297.

In another embodiment, the additional therapeutic agent is a sodium channel inhibitor (also known as a sodium channel blocker), such as the Naᵥ1.7 and Naᵥ1.8 blockers identified above.

The amount of additional therapeutic agent present in the compositions of this invention may be no more than the amount that would normally be administered in a composition comprising that therapeutic agent as the only active agent. The amount of additional therapeutic agent in the presently disclosed compositions may range from about 10% to 100% of the amount normally present in a composition comprising that agent as the only therapeutically active agent.

The compounds and salts of this invention or pharmaceutically acceptable compositions thereof may also be incorporated into compositions for coating an implantable medical device, such as prostheses, artificial valves, vascular grafts, stents and catheters. Accordingly, the invention, in another aspect, includes a composition for coating an implantable device comprising a compound or salt of the invention as described generally above, and in classes and subclasses herein, and a carrier suitable for coating said implantable device. In still another aspect, the invention includes an implantable device coated with a composition comprising a compound or salt of the invention as described generally above, and in classes and subclasses herein, and a carrier suitable for coating said implantable device. Suitable coatings and the general preparation of coated implantable devices are described in U.S. Pat. Nos. 6,099,562, 5,886,026, and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

Another aspect of the invention relates to inhibiting Naᵥ1.8 activity in a biological sample or a subject, which method comprises administering to the subject, or contacting said biological sample with a compound of the invention, a pharmaceutically acceptable salt thereof, or a pharmaceutical composition thereof. The term “biological sample,” as used herein, includes, without limitation, cell cultures or extracts thereof, biopsied material obtained from a mammal or extracts thereof, and blood, saliva, urine, feces, semen, tears, or other body fluids or extracts thereof.

Inhibition of Naᵥ1.8 activity in a biological sample is useful for a variety of purposes that are known to one of skill in the art. Examples of such purposes include, but are not limited to, the study of sodium channels in biological and pathological phenomena, and the comparative evaluation of new sodium channel inhibitors.

### Synthesis of the Compounds of the Invention

The compounds of the invention can be prepared from known materials by the methods described in the Examples, other similar methods, and other methods known to one skilled in the art. As one skilled in the art would appreciate, the functional groups of the intermediate compounds in the methods described below may need to be protected by suitable protecting groups. Protecting groups may be added or removed in accordance with standard techniques, which are well-known to those skilled in the art. The use of protecting groups is described in detail in T. G. M. Wuts et al., Greene's Protective Groups in Organic Synthesis (4th ed. 2006).

### Radiolabeled Analogs of the Compounds of the Invention

In another aspect, the invention relates to radiolabeled analogs of the compounds of the invention. As used herein, the term “radiolabeled analogs of the compounds of the invention” refers to compounds that are identical to the compounds of the invention, as described herein, including all embodiments thereof, except that one or more atoms has been replaced with a radioisotope of the atom present in the compounds of the invention.

As used herein, the term “radioisotope” refers to an isotope of an element that is known to undergo spontaneous radioactive decay. Examples of radioisotopes include ³H, ¹⁴C, ³²P, ³⁵S, ¹⁸F, ³⁶Cl, and the like, as well as the isotopes for which a decay mode is identified in V. S. Shirley & C. M. Lederer, Isotopes Project, Nuclear Science Division, Lawrence Berkeley Laboratory, Table of Nuclides (January 1980).

The radiolabeled analogs can be used in a number of beneficial ways, including in various types of assays, such as substrate tissue distribution assays. For example, tritium (3H)- and/or carbon-14 (14° C.)-labeled compounds may be useful for various types of assays, such as substrate tissue distribution assays, due to relatively simple preparation and excellent detectability.

In another aspect, the invention relates to pharmaceutically acceptable salts of the radiolabeled analogs, in accordance with any of the embodiments described herein in connection with the compounds of the invention.

In another aspect, the invention relates to pharmaceutical compositions comprising the radiolabeled analogs, or pharmaceutically acceptable salts thereof, and a pharmaceutically acceptable carrier, adjuvant or vehicle, in accordance with any of the embodiments described herein in connection with the compounds of the invention.

In another aspect, the invention relates to methods of inhibiting voltage-gated sodium channels and methods of treating or lessening the severity of various diseases and disorders, including pain, in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to radiolabeled analogs, pharmaceutically acceptable salts thereof, and pharmaceutical compositions thereof, for use, in accordance with any of the embodiments described herein in connection with the compounds of the invention.

In another aspect, the invention relates to the use of the radiolabeled analogs, or pharmaceutically acceptable salts thereof, and pharmaceutical compositions thereof, for the manufacture of medicaments, in accordance with any of the embodiments described herein in connection with the compounds of the invention.

In another aspect, the radiolabeled analogs, pharmaceutically acceptable salts thereof, and pharmaceutical compositions thereof, can be employed in combination therapies, in accordance with any of the embodiments described herein in connection with the compounds of the invention.

### ENUMERATED EMBODIMENTS

Additional embodiments, features, and advantages of the disclosure will be apparent from the following detailed description and through practice of the disclosure. The compounds and methods of the present disclosure can be described as embodiments in any of the following enumerated clauses. It will be understood that any of the embodiments described herein can be used in connection with any other embodiments described herein to the extent that the embodiments do not contradict one another.
1. A compound of formula (I)

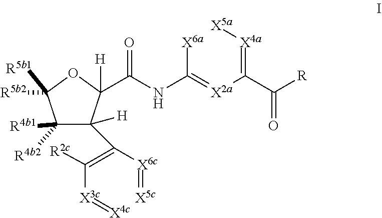

or a pharmaceutically acceptable salt thereof, wherein:
X^{2a} is N, N⁺—O, or C—R^{2a};
X^{4a} is N, N⁺—O, or C—R^{4a};
X^{5a} is N, N⁺—O, or C—R^{5a};
X^{6a} is N, N⁺—O, or C—R^{6a}; R is OR^{a} or NR^{Xa}R^{Ya};
R^{2a}, R^{4a}, R^{5a}, and R^{6a} are each independently H, halo, C₁-C₆ alkyl, C₁-C₆ haloalkyl, or —Si(C₁-C₆ alkyl)₃;
R^{a} is H or C₁-C₆ alkyl;
R^{Xa} is H or C₁-C₆ alkyl;
R^{Ya} is H, OH, C₁-C₆ alkyl, —(C₁-C₆ alkylene)-R^{Za1}, or 4-6 membered heterocyclyl optionally substituted with one or more groups independently selected from C₁-C₆ alkyl and C₁-C₆ alkoxy;
or R^{Xa} and R^{Ya}, together with the nitrogen atom to which they are attached, form a 5-9 membered heterocyclyl, wherein said heterocyclyl is optionally substituted with one or more R^{Za2};
R^{Za1} is OH, NH₂, —NH(C₁-C₆ alkyl), —N(C₁-C₆ alkyl)₂, and 5-6 membered heterocyclyl optionally substituted with one or more groups independently selected from halo and C₁-C₆ alkyl;
each R^{Za2} is independently selected from halo, OH, C₁-C₆ alkyl, C₁-C₆ alkoxy, NH₂, —NH(C₁-C₆ alkyl), —N(C₁-C₆ alkyl)₂, and —(C₁-C₆ alkylene)-(C₁-C₆ alkoxy);
R^{4b1} and R^{4b2} are each independently H, C₁-C₆ alkyl, C₃-C₆ cycloalkyl, or C₁-C₆ haloalkyl;
R^{5b1} and R^{5b2} are each independently H, C₁-C₆ alkyl, C₃-C₆ cycloalkyl, C₁-C₆ haloalkyl, or —(C₁-C₆ alkylene)-(C₁-C₆ alkoxy);
or R^{5b1} and R^{5b2}, together with the carbon atom to which they are attached, form a 4-6 membered heterocyclyl;
X^{3c} is N or C—R^{3c};
X^{4c} is N or C—R^{4c};
X^{5c} is N or C—R^{5c};
X^{6c} is N or C—R⁶⁰;
R^{2c} is H, OH, halo, C₁-C₆ alkyl, C₂-C₆ alkenyl, C₁-C₆ haloalkyl, C₁-C₆ alkoxy, C₁-C₆ haloalkoxy, —(C₁-C₆ alkylene)-(C₁-C₆ alkoxy), —(C₁-C₆ alkylene)-O-(4-6 membered heterocyclyl), —O—(C₂-C₆ alkenylene)-(C₁-C₆ haloalkyl), -L¹-L²-(C₃-C₇ cycloalkyl), or —O-L³-R^{Xc}, wherein said cycloalkyl is optionally substituted with one or more groups independently selected from halo, OH, CN, C₁-C₆ alkyl, C₁-C₆ alkoxy, ═NOH, —C(O) (C₁-C₆ alkyl), and —(C₁-C₆ alkylene)-OH;
L¹ is a bond or O;
L² is a bond or C₁-C₆ alkylene;
L³ is a bond, C₁-C₆ alkylene, or C₂-C₆ alkenylene;
R^{Xc} is selected from OH, CN, C₁-C₆ alkoxy, NH₂, —NH(C₁-C₆ alkyl), —N(C₁-C₆ alkyl)₂, —NH(C₁-C₆ haloalkyl), —NH(C₁-C₆ haloalkyl)₂, —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O) (C₁-C₆ alkyl), —C(O) NH₂, —C(O) NH(C₁-C₆ alkyl), —C(O) N(C₁-C₆ alkyl)₂, —NH (4-6 membered heterocyclyl), ═NOH, ═NO(C₁-C₆ alkyl), —N═S(O)(C₁-C₆ alkyl)₂, —C(═NOH)(C₃-C₆ cycloalkyl), 4-8 membered heterocyclyl, and 5-6 membered heteroaryl, wherein said cycloalkyl is optionally substituted with one or more halo, and wherein said heterocyclyl and heteroaryl are optionally substituted with one or more groups independently selected from OH, halo, C₁-C₆ alkyl, C₁-C₆ alkoxy, C₁-C₆ haloalkyl, C₁-C₆ haloalkoxy, and —(C₁-C₆ alkylene)-OH;
R^{3c} is H, halo, C₁-C₆ alkyl, C₁-C₆ haloalkyl, —(C₁-C₆ alkylene)-OH, or —(C₁-C₆ alkylene)-(C₁-C₆ alkoxy);
or wherein X^{3c} is C—R^{3c}, and R^{2c} and R^{3c}, together with the carbon atoms to which they are attached, form a ring of formula:

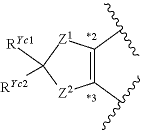

Z¹ and Z² are each, independently, O, CH₂, or CF₂;
R^{Yc1} and R^{Yc2} are each, independently, H or halo;
R^{4c} is H, halo, OH, —OBn, C₁-C₆ alkyl, C₁-C₆ alkoxy, C₁-C₆ haloalkyl, C₁-C₆ haloalkoxy, or -L¹-L²-(C₃-C₆ cycloalkyl), wherein said cycloalkyl is optionally substituted with 1-2 halo;
R^{5c} is H, halo, OH, —OBn, C₁-C₆ alkyl, C₁-C₆ haloalkyl, or -L¹-L²-(C₃-C₆ cycloalkyl), wherein said cycloalkyl is optionally substituted with 1-2 halo;
R^{6c} is H, halo, C₁-C₆ alkyl, or C₁-C₆ haloalkyl;
provided that no more than two of X^{2a}, X^{4a}, X^{5a}, and X^{6a} are N or N⁺—O;
provided that no more than one of X^{3c}, X^{4c}, X^{5c}, and X^{6c} is N; and
provided that:
R is OR^{a}; or
R is NR^{Xa}R^{Ya}, wherein R^{Ya} is OH, —(C₁-C₆ alkylene)-R^{Za1}, or 4-6 membered heterocyclyl optionally substituted with one or more groups independently selected from C₁-C₆ alkyl and C₁-C₆ alkoxy; or
R is NR^{Xa}R^{Ya}, wherein R^{Xa} and R^{Ya}, together with the N atom to which they are attached, form a 5-9 membered heterocyclyl, and wherein said heterocyclyl is optionally substituted with one or more R^{Za2}; or
R^{2a}, R^{4a}, R^{5a}, or R^{6a} is —Si(C₁-C₆ alkyl); or
R^{5b1} or R^{5b2} is —(C₁-C₆ alkylene)-(C₁-C₆ alkoxy); or
R^{5b1} and R^{5b2}, together with the carbon atom to which they are attached, form a 4-6 membered heterocyclyl; or
R^{2c} is —(C₁-C₆ alkylene)-(C₁-C₆ alkoxy), —(C₁-C₆ alkylene)-O-(4-6 membered heterocyclyl), —O—(C₂-C₆ alkenylene)-(C₁-C₆ haloalkyl), or —O-L³-R^{Xc}; or
R^{2c} is -L¹-L²-(C₃-C₇ cycloalkyl), wherein said cycloalkyl is substituted with one or more groups independently selected from OH, CN, C₁-C₆ alkyl, C₁-C₆ alkoxy, ═NOH, —C(O) (C₁-C₆ alkyl), and —(C₁-C₆ alkylene)-OH; or
R^{3c} is —(C₁-C₆ alkylene)-OH or —(C₁-C₆ alkylene)-(C₁-C₆ alkoxy); or
R^{2c} and R^{3c}, together with the carbon atoms to which they are attached, form a ring of formula:

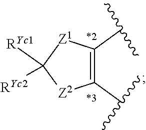

or
R^{4c} is OH, —OBn, C₁-C₆ alkoxy, C₁-C₆ haloalkoxy, or -L¹-L²-(C₃-C₆ cycloalkyl), wherein said cycloalkyl is optionally substituted with 1-2 halo; or
R^{5c} is OH, —OBn, or -L¹-L²-(C₃-C₆ cycloalkyl), wherein said cycloalkyl is optionally substituted with 1-2 halo.
2. The compound of clause 1, wherein the compound has formula (I-A)

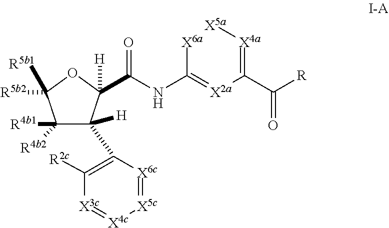

or a pharmaceutically acceptable salt thereof.
3. The compound of clause 1 or 2, wherein the compound has formula (I-A-1)

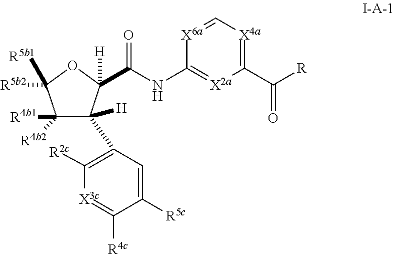

or a pharmaceutically acceptable salt thereof.
4. The compound of clause 1 or 2, wherein the compound has formula (I-B)

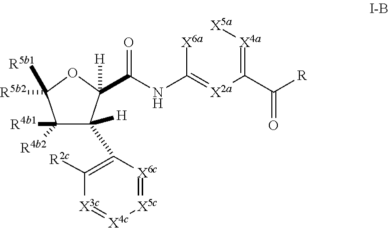

or a pharmaceutically acceptable salt thereof.
5. The compound of any one of clauses 1 to 4, wherein the compound has formula (I-B-1)

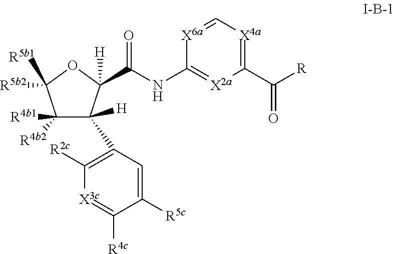

or a pharmaceutically acceptable salt thereof.
6. The compound of clause 1 or 2, wherein the compound has formula (I-C)

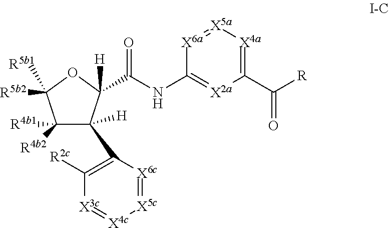

or a pharmaceutically acceptable salt thereof.
7. The compound of any one of clauses 1 to 3 or 6, wherein the compound has formula (I-C-1)

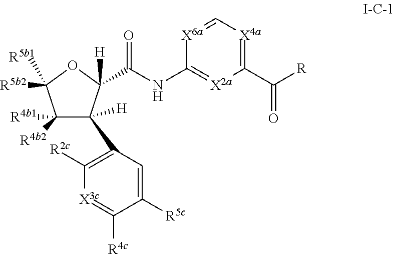

or a pharmaceutically acceptable salt thereof.
8. The compound of any one of clauses 1 to 7, or the pharmaceutically acceptable salt thereof, wherein X^{2a} is N or C—R^{2a}; and R^{2a} is H.
9. The compound of clause 8, or the pharmaceutically acceptable salt thereof, wherein X^{2a} is N.
10. The compound of clause 8, or the pharmaceutically acceptable salt thereof, wherein X^{2a} is C—R^{2a}; and R^{2a} is H.
11. The compound of any one of clauses 1 to 10, or the pharmaceutically acceptable salt thereof, wherein X^{4a} is N, N⁺—O, or C—R^{4a}; and R^{4a} is H or halo.
12. The compound of clause 11, or the pharmaceutically acceptable salt thereof, wherein X^{4a} is N.
13. The compound of clause 11, or the pharmaceutically acceptable salt thereof, wherein X^{4a} is N⁺—O.
14. The compound of clause 11, or the pharmaceutically acceptable salt thereof, wherein X^{4a} is C—R^{4a}; and R^{4a} is H or F.
15. The compound of any one of clauses 1, 2, 4, 6, or 8 to 14, or the pharmaceutically acceptable salt thereof, wherein X^{5a} is C—R^{5a}; and R^{5a} is H.
16. The compound of any one of clauses 1 to 15, or the pharmaceutically acceptable salt thereof, wherein X^{6a} is N or C—R^{6a}; and R^{6a} is H, halo, C₁-C₆ alkyl, or —Si(C₁-C₆ alkyl)₃.
17. The compound of clause 16, or the pharmaceutically acceptable salt thereof, wherein X^{6a} is N.
18. The compound of clause 16, or the pharmaceutically acceptable salt thereof, wherein X^{6a} is C—R^{6a}; and R^{6a} is H, F, CH₃, or —Si(CH₃)₃.
19. The compound of any one of clauses 1 to 18, or the pharmaceutically acceptable salt thereof, wherein:
R is OR^{a} or NR^{Xa}R^{Ya};
R^{a} is H or C₁-C₆ alkyl;
R^{Xa} is H or C₁-C₆ alkyl;
R^{Ya} is H, OH, C₁-C₆ alkyl, —(C₁-C₆ alkylene)-R^{Za1}, or 4-6 membered heterocyclyl optionally substituted with one or more groups independently selected from C₁-C₆ alkyl and C₁-C₆ alkoxy; and
R^{Za1} is OH, —NH(C₁-C₆ alkyl), —N(C₁-C₆ alkyl)₂, and 5-6 membered heterocyclyl optionally substituted with one or more groups independently selected from halo and C₁-C₆ alkyl.
20. The compound of clause 19, or the pharmaceutically acceptable salt thereof, wherein R is OR^{a}; and R^{a} is H.
21. The compound of clause 19, or the pharmaceutically acceptable salt thereof, wherein:
R is NR^{Xa}R^{Ya};
R^{Xa} is H or CH₃;
R^{Ya} is H, OH, CH₃, —(C₁-C₂ alkylene)-R^{Za1}, or 4-6 membered heterocyclyl optionally substituted with one or more groups independently selected from CH₃, —OCH₃, and —OCH₂CH₃; and
R^{Za1} is OH, —NH(CH₃), —N(CH₃)₂, and 5-6 membered heterocyclyl optionally substituted with one or more groups independently selected from F and CH₃.
22. The compound of any one of clauses 1 to 18, or the pharmaceutically acceptable salt thereof, wherein:
R is NR^{Xa}R^{Ya}.
R^{Xa} and R^{Ya}, together with the nitrogen atom to which they are attached, form a 5-9 membered heterocyclyl optionally substituted with one or more R^{Za2}; and
each R^{Za2} is independently selected from halo, OH, C₁-C₆ alkyl, C₁-C₆ alkoxy, NH₂, —NH(C₁-C₆ alkyl), —N(C₁-C₆ alkyl)₂, and —(C₁-C₆ alkylene)-(C₁-C₆ alkoxy).
23. The compound of clause 22, or the pharmaceutically acceptable salt thereof, wherein each R^{Za2} is independently selected from F, OH, CH₃, —OCH₃, NH₂, —NH(CH₃), —N(CH₃)₂, and —CH₂OCH₃.
24. The compound of any one of clauses 1 to 23, or the pharmaceutically acceptable salt thereof, wherein R^{4b1} is H or C₁-C₆ alkyl.
25. The compound of clause 24, or the pharmaceutically acceptable salt thereof, wherein R^{4b1} is H or CH₃.
26. The compound of any one of clauses 1 to 25, or the pharmaceutically acceptable salt thereof, wherein R^{4b2} is H or C₁-C₆ alkyl.
27. The compound of clause 26, or the pharmaceutically acceptable salt thereof, wherein R^{4b2} is H or CH₃.
28. The compound of any one of clauses 1 to 27, or the pharmaceutically acceptable salt thereof, wherein R^{5b1} is C₁-C₆ alkyl, C₁-C₆ haloalkyl, or —(C₁-C₆ alkylene)-(C₁-C₆ alkoxy).
29. The compound of clause 28, or the pharmaceutically acceptable salt thereof, wherein R^{5b1} is CH₃, CF₃, —CH₂OCH₃, or —CH₂CH₂OCH₃.
30. The compound of any one of clauses 1 to 29, or the pharmaceutically acceptable salt thereof, wherein R⁵⁶² is C₁-C₆ alkyl, C₁-C₆ haloalkyl, or —(C₁-C₆ alkylene)-(C₁-C₆ alkoxy).
31. The compound of clause 30, or the pharmaceutically acceptable salt thereof, wherein R^{5b2} is CH₃, CF₃, —CH₂OCH₃, or —CH₂CH₂OCH₃.
32. The compound of any one of clauses 1 to 27, or the pharmaceutically acceptable salt thereof, wherein R^{5b1} and R^{5b2}, together with the carbon atom to which they are attached, form a 4-membered heterocyclyl.
33. The compound of any one of clauses 1 to 32, or the pharmaceutically acceptable salt thereof, wherein X^{3c} is N.
34. The compound of any one of clauses 1 to 32, or the pharmaceutically acceptable salt thereof, wherein X^{3c} is C—R^{3c}; and R^{3c} is H, halo, C₁-C₆ alkyl, C₁-C₆ haloalkyl, —(C₁-C₆ alkylene)-OH, or —(C₁-C₆ alkylene)-(C₁-C₆ alkoxy).
35. The compound of clause 34, or the pharmaceutically acceptable salt thereof, wherein R^{3c} is H, F, C₁, CH₃, CF₃, —CH₂OH, or —CH₂OCH₃.
36. The compound of any one of clauses 1, 2, 4, 6, or 8 to 35, or the pharmaceutically acceptable salt thereof, wherein X^{4c} is C—R^{4c}; R^{4c} is H, halo, OH, —OBn, C₁-C₆ alkoxy, C₁-C₆ haloalkyl, C₁-C₆ haloalkoxy, or -L¹-L²-(C₃-C₆ cycloalkyl), wherein said cycloalkyl is optionally substituted with 1-2 halo; L¹ is O; and L² is a bond or C₁-C₆ alkylene.
37. The compound of any one of clauses 3, 5, 7, or 8 to 35, or the pharmaceutically acceptable salt thereof, wherein R^{4c} is H, halo, OH, —OBn, C₁-C₆ alkoxy, C₁-C₆ haloalkyl, C₁-C₆ haloalkoxy, or -L¹-L²-(C₃-C₆ cycloalkyl), wherein said cycloalkyl is optionally substituted with 1-2 halo; L¹ is O; and L² is a bond or C₁-C₆ alkylene.
38. The compound of clause 36 or 37, wherein R^{4c} is H, F, OH, —OBn, —OCH₃, —OCH₂CH₃, CHF₂, —OCHF₂, —OCF₃, —O—CH₂-(cyclopropyl), or —O-(cyclobutyl), wherein said cyclobutyl is substituted with 2 F.
39. The compound of any one of clauses 1, 2, 4, 6, or 8 to 38, or the pharmaceutically acceptable salt thereof, wherein X^{5c} is C—R^{5c}; and R^{5c} is H, halo, OH, —OBn, or -L¹-L²-(C₃-C₆ cycloalkyl), wherein said cycloalkyl is optionally substituted with 1-2 halo; L¹ is O; and L² is a bond.
40. The compound of any one of clauses 3, 5, 7, or 8 to 38, or the pharmaceutically acceptable salt thereof, wherein R^{5c} is H, halo, OH, —OBn, or -L¹-L²-(C₃-C₆ cycloalkyl), wherein said cycloalkyl is optionally substituted with 1-2 halo; L¹ is O; and L² is a bond.
41. The compound of clause 39 or 40, or the pharmaceutically acceptable salt thereof, wherein R^{5c} is H, Cl, OH, —OBn, or —O-(cyclobutyl), wherein said cyclobutyl is substituted with 2 F.
42. The compound of any one of clauses 1, 2, 4, 6, or 8 to 41, or the pharmaceutically acceptable salt thereof, wherein X^{6c} is C—R^{6c}; and R^{6c} is H.
43. The compound of any one of clauses 1 to 42, or the pharmaceutically acceptable salt thereof, wherein:
R^{2c} is OH, halo, C₁-C₆ alkoxy, —(C₁-C₆ alkylene)-(C₁-C₆ alkoxy), —(C₁-C₆ alkylene)-O-(4-6 membered heterocyclyl), —O—(C₂-C₆ alkenylene)-(C₁-C₆ haloalkyl), -L¹-L²-(C₃-C₇ cycloalkyl), or —O-L³-R^{Xc}, wherein said cycloalkyl is optionally substituted with one or more groups independently selected from OH, CN, C₁-C₆ alkyl, C₁-C₆ alkoxy, ═NOH, C(O)(C₁-C₆ alkyl), and —(C₁-C₆ alkylene)-OH;
L¹ is O;
L² is a bond or C₁-C₆ alkylene;
L³ is a bond, C₁-C₆ alkylene, or C₂-C₆ alkenylene; and
R^{Xc} is selected from OH, CN, C₁-C₆ alkoxy, NH₂, —NH(C₁-C₆ alkyl), —N(C₁-C₆ alkyl)₂, —NH(C₁-C₆ haloalkyl), —NH(C₁-C₆ haloalkyl)₂, —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O) (C₁-C₆ alkyl), —C(O) NH₂, —C(O) NH(C₁-C₆ alkyl), —C(O) N(C₁-C₆ alkyl)₂, —NH (4-6 membered heterocyclyl), ═NOH, ═NO(C₁-C₆ alkyl), —N═S(O)(C₁-C₆ alkyl)₂, —C(═NOH)(C₃-C₆ cycloalkyl), 4-8 membered heterocyclyl, and 5-6 membered heteroaryl, wherein said cycloalkyl is optionally substituted with one or more halo, and wherein said heterocyclyl and heteroaryl are optionally substituted with one or more groups independently selected from OH, halo, C₁-C₆ alkyl, C₁-C₆ alkoxy, C₁-C₆ haloalkyl, C₁-C₆ haloalkoxy, and —(C₁-C₆ alkylene)-OH.
44. The compound of clause 43, or the pharmaceutically acceptable salt thereof, wherein R^{2c} is OH, Cl, —OCH₃, —CH₂OCH₃, —CH₂—O-(4-membered heterocyclyl), or —O—(C₃-C₄ alkenylene)-CF₃.
45. The compound of clause 43, or the pharmaceutically acceptable salt thereof, wherein:
R^{2c} is -L¹-L²-(C₃-C₇ cycloalkyl);
L¹ is O; and
L² is a bond or C₁-C₂ alkylene,
and wherein said cycloalkyl is substituted with one or more groups independently selected from OH, CN, —OCH₃, CH₃, ═NOH, —C(O)(CH₃), and —CH₂OH.
46. The compound of clause 43, or the pharmaceutically acceptable salt thereof, wherein:
R^{2c} is —O-L³-R^{Xc};
L³ is a bond, C₁-C₆ alkylene, or C₄-C₅ alkenylene; and
R^{Xc} is selected from OH, CN, C₁-C₆ alkoxy, NH₂, —NH(C₁-C₆ alkyl), —N(C₁-C₆ alkyl)₂, —NH(C₁-C₆ haloalkyl), —NH(C₁-C₆ haloalkyl)₂, —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O) (C₁-C₆ alkyl), —C(O) NH₂, —C(O) NH(C₁-C₆ alkyl), —C(O) N(C₁-C₆ alkyl)₂, —NH (4-6 membered heterocyclyl), ═NOH, ═NO(C₁-C₆ alkyl), —N═S(O)(C₁-C₆ alkyl)₂, —C(═NOH)(C₃-C₆ cycloalkyl), 4-8 membered heterocyclyl, and 5-6 membered heteroaryl, wherein said cycloalkyl is optionally substituted with one or more halo, and wherein said heterocyclyl and heteroaryl are optionally substituted with one or more groups independently selected from OH, halo, C₁-C₆ alkyl, C₁-C₆ alkoxy, C₁-C₆ haloalkyl, C₁-C₆ haloalkoxy, and —(C₁-C₆ alkylene)-OH.
47. The compound of clause 46, or the pharmaceutically acceptable salt thereof, wherein R^{Xc} is selected from OH, CN, —OCH₃, —NH(CH₃), —NH(CH(CH₃)₂), —N(CH₃)₂, —NH(CH₂CHF₂), —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O)(CH₃), —C(O) NH(CH₃), —NH (4-5 membered heterocyclyl), ═NOH, ═NO(CH₃), —N═S(O)(CH₃)₂, —C(═NOH)(C₃-C₄ cycloalkyl), 4-8 membered heterocyclyl optionally substituted with one or more groups independently selected from OH, F, CH₃, —OCH₃, CHF₂, CF₃, —OCHF₂, and —CH₂OH, and 5-membered heteroaryl optionally substituted with CH₃, and wherein said cycloalkyl is optionally substituted with one F.
48. The compound of any one of clauses 1 to 42, or the pharmaceutically acceptable salt thereof, wherein X^{3c} is C—R^{3c}, and R^{2c} and R^{3c}, together with the carbon atoms to which they are attached, form a ring of formula:

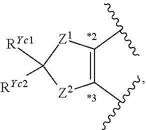

and wherein:
Z¹ is O or CH₂;
Z² is O or CF₂; and
R^{Yc1} and R^{Yc2} are each, independently, H or F.
49. The compound of clause 48, or the pharmaceutically acceptable salt thereof, wherein the ring is of formula:

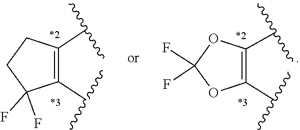 [SMILES: *C1=C(*)C(F)(F)CC1 ; *C1=C(*)OC(F)(F)O1]

50. A compound selected from Table A or Table B, or a pharmaceutically acceptable salt thereof.
51. The compound of any one of clauses 1 to 50 in non-salt form.
52. A pharmaceutical composition comprising a therapeutically effective amount of the compound of any one of clauses 1-50, or a pharmaceutically acceptable salt thereof, or the compound of clause 51, and one or more pharmaceutically acceptable carriers or vehicles.
53. A pharmaceutical composition comprising the compound of any one of clauses 1-50, or a pharmaceutically acceptable salt thereof, or the compound of clause 51, and one or more pharmaceutically acceptable carriers or vehicles.
54. A method of inhibiting a voltage-gated sodium channel in a subject comprising administering to the subject the compound of any one of clauses 1-50, or a pharmaceutically acceptable salt thereof, the compound of clause 51, or the pharmaceutical composition of clause 52 or 53.
55. The method of clause 54, wherein the voltage-gated sodium channel is Naᵥ1.8.
56. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of any one of clauses 1-50, or a pharmaceutically acceptable salt thereof, the compound of clause 51, or the pharmaceutical composition of clause 52 or 53.
57. The method of clause 56, where the method comprises treating or lessening the severity in the subject of neuropathic pain.
58. The method of clause 56, wherein the neuropathic pain comprises post-herpetic neuralgia.
59. The method of clause 56, wherein the neuropathic pain comprises small-fiber neuropathy.
60. The method of clause 56, wherein the neuropathic pain comprises idiopathic small-fiber neuropathy.
61. The method of clause 56, wherein the neuropathic pain comprises diabetic neuropathy.
62. The method of clause 61, wherein the diabetic neuropathy comprises diabetic peripheral neuropathy.
63. The method of clause 56, wherein the method comprises treating or lessening the severity in the subject of musculoskeletal pain.
64. The method of clause 63, wherein the musculoskeletal pain comprises osteoarthritis pain.
65. The method of clause 56, wherein the method comprises treating or lessening the severity in the subject of acute pain.
66. The method of clause 65, wherein the acute pain comprises acute post-operative pain.
67. The method of clause 56, wherein the method comprises treating or lessening the severity in the subject of postsurgical pain.
68. The method of clause 67, wherein the postsurgical pain comprises bunionectomy pain. 69. The method of clause 67, wherein the postsurgical pain comprises abdominoplasty pain.
70. The method of clause 67, wherein the postsurgical pain comprises herniorrhaphy pain.
71. The method of clause 56, wherein the method comprises treating or lessening the severity in the subject of visceral pain.
72. The method of any one of clauses 54-71, wherein said subject is treated with one or more additional therapeutic agents administered concurrently with, prior to, or subsequent to treatment with the compound, pharmaceutically acceptable salt, or pharmaceutical composition.
73. Use of the compound of any one of clauses 1-50, or a pharmaceutically acceptable salt thereof, the compound of clause 51, or the pharmaceutical composition of clause 52 or 53, as a medicament.

### EXAMPLES

General methods. ¹H NMR spectra were obtained as solutions in an appropriate deuterated solvent such as dimethyl sulfoxide-d₆ (DMSO-d6).

Compound purity, retention time, and electrospray mass spectrometry (ESI-MS) data were determined by LC/MS analysis. LC/MS analysis was conducted using an Acquity UPLC BEH C₈ column (50×2.1 mm, 1.7 μm particle) made by Waters (pn: 186002877) with a (2.1×5 mm, 1.7 μm particle) guard column (pn: 186003978), and a dual gradient run from 2-98% mobile phase B over 4.45 minutes. Mobile phase A=H₂O (10 mM ammonium formate with 0.05% ammonium hydroxide). Mobile phase B=acetonitrile. Flow rate=0.6 mL/min, injection volume=2 μL, and column temperature=45° C.

X-ray powder diffraction analysis method: X-ray powder diffraction (XRPD) analysis was performed at room temperature in transmission mode using a PANalytical Empyrean system equipped with a sealed tube source and a PIXcel 3D Medipix-3 detector (Malvern PANalytical Inc, Westborough, Massachusetts). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40° 20 with a step size of 0.0131303° and 49s per step.

### Abbreviations

Unless otherwise noted, or where the context dictates otherwise, the following abbreviations shall be understood to have the following meanings:

| Abbreviation | Meaning |
|---|---|
| NMR | Nuclear magnetic resonance |
| ESI-MS | Electrospray mass spectrometry |
| LC/MS | Liquid chromatography-mass spectrometry |
| UPLC | Ultra performance liquid chromatography |
| HPLC/MS/MS | High performance liquid chromatography/tandem mass spectrometry |
| IS | Internal standard |
| HPLC | High performance liquid chromatography |
| SFC | Supercritical fluid chromatography |
| ESI | Electrospray ionization |
| kg | Kilogram |
| g | Grams |
| mg | Milligrams |
| L | Liter(s) |
| mL | Milliliters |
| μL | Microliters |
| nL | Nanoliters |
| mol | Mole |
| mmol | Millimoles |
| hr, h | Hours |
| min | Minutes |
| ms | Millisecond |
| mm | Millimeters |
| μm | Micrometers |
| nm | Nanometer |
| MHz | Megahertz |
| Hz | Hertz |
| N | Normal (concentration) |
| M | Molar (concentration) |
| mM | Millimolar (concentration) |
| μM | Micromolar (concentration) |
| ppm | Parts per million |
| % w/v | Weight-volume concentration |
| % w/w | Weight-weight concentration |
| Ac₂O | Acetic anhydride |
| BnBr | Benzyl bromide |
| t-BuOH | Tert-butyl alcohol |
| CDI | 1,1′-Carbonyldiimidazole |
| DAST | (Diethylamino)sulfur trifluoride |
| DCM | Dichloromethane |
| DCE | Dichloroethane |
| DIAD | Diisopropyl azodicarboxylate |
| DIBAL | Diisobutylaluminium hydride |
| DIEA, DIPEA | N,N-Diisopropyl ethyl amine |
| DMA | N,N-Dimethylacetamide |
| DMAP | Dimethylaminopyridine |
| DMF | N,N-Dimethylformamide |
| DMSO | Dimethyl sulfoxide |
| DRG | Dorsal root ganglia |
| EtOH | Ethanol |
| EtOAc | Ethyl acetate |
| HATU | 1-[Bis(dimethylamino)methylene]-1H-1,2,3-triazolo[4,5-b]pyridinium |
| | 3-oxide hexafluorophosphate |
| EDCI | 1-Ethyl-3-(3-dimethylaminopropyl)carbodiimide |
| T3P | Propylphosphonic anhydride, i.e., 2,4,6-tripropyl-1,3,5,2,4,6- |
| | trioxatriphosphinane 2,4,6-trioxide |
| mCPBA | Meta-Chloroperoxybenzoic acid |
| MeOH | Methanol |
| MsCl | Methanesulfonyl chloride |
| MTBE | Methyl tert-butyl ether |
| NCS | N-Chlorosuccinimide |
| NIS | N-Iodosuccinimide |
| NMP | N-Methylpyrrolidone |
| PTSA | Para-toluenesulfonic acid |
| STAB | Sodium triacetoxyborohydride |
| TBAF | Tetrabutylammonium fluoride |
| TBSOTf | Tert-Butyldimethylsilyl trifluoromethanesulfonate |
| TCFH | Chloro-N,N,N′,N′-tetramethylformamidinium hexafluorophosphate |
| TEP | Ethyltriphenylphosphonium bromide |
| THF | Tetrahydrofuran |
| TEA | Triethylamine |
| Tf₂O | Trifluoromethanesulfonic anhydride |
| TFA | Trifluoroacetic acid |
| TMSCl | Trimethylsilyl chloride |
| TMSCN | Trimethylsilyl cyanide |
| RB | Round bottom (flask) |
| RT | Room temperature |
| ca. | Circa (approximately) |
| E-VIPR | Electrical stimulation voltage ion probe reader |
| HEK | Human embryonic kidney |
| KIR2.1 | Inward-rectifier potassium ion channel 2.1 |
| DMEM | Dulbecco's Modified Eagle's Medium |
| FBS | Fetal bovine serum |
| NEAA | Non-essential amino acids |
| HEPES | 2-[4-(2-hydroxyethyl)piperazin-1-yl]ethanesulfonic acid |
| DiSBAC₆(3) | Bis-(1,3-dihexyl-thiobarbituric acid) trimethine oxonol |
| CC2-DMPE | Chlorocoumarin-2-dimyristoyl phosphatidylethanolamine |
| VABSC-1 | Voltage Assay Background Suppression Compound |
| HS | Human serum |
| BSA | Bovine Serum Albumin |

### Example 1

### rel-4-((2S,3R,4R,5S)-3-(3,4-difluoro-2-(2-methoxyethoxy)phenyl)-4,5-dimethyl-5-(trifluoromethyl)tetrahydrofuran-2-carboxamido) picolinamide (1) and

### rel-4-((2R,3S,4S,5R)-3-(3,4-difluoro-2-(2-methoxyethoxy)phenyl)-4,5-dimethyl-5-(trifluoromethyl)tetrahydrofuran-2-carboxamido) picolinamide (2)

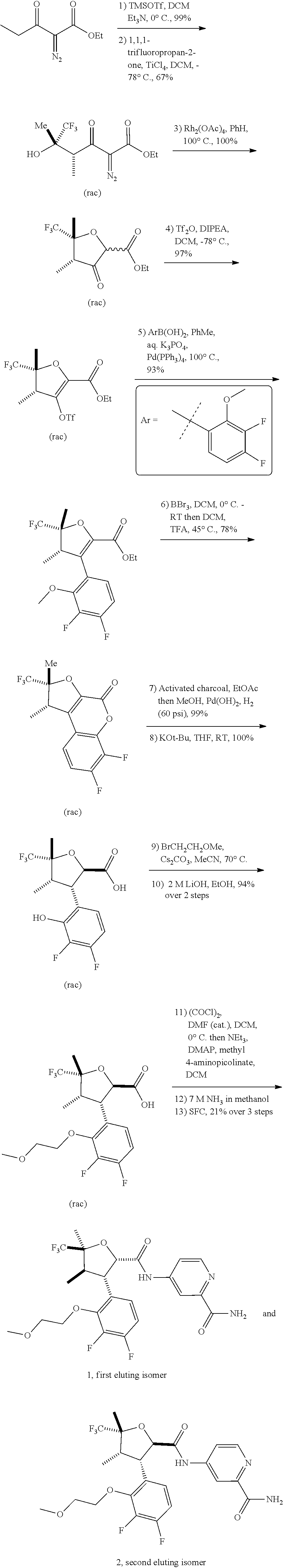

### Step 1:

Et₃N (7.7 mL, 55.2 mmol) was added to a stirred solution of ethyl 2-diazo-3-oxo-pentanoate (6.69 g, 39.3 mmol) in DCM (80 mL) at 0° C. under nitrogen. Trimethylsilyl trifluoromethanesulfonate (8.5 mL, 47.0 mmol) was added dropwise over 5 min and the mixture was stirred for a further 30 min at 0° C. The reaction mixture was diluted with pentane (100 mL), the layers separated and the organic phase washed with dilute aqueous sodium bicarbonate (100 mL) and then brine (100 mL). The organic layer was dried (MgSO₄), and concentrated in vacuo to give ethyl (Z)-2-diazo-3-trimethylsilyloxypent-3-enoate (9.4 g, 99%) as a red oil. ¹H NMR (500 MHZ, Chloroform-d) δ 5.33 (q, J=7.0 Hz, 1H), 4.25 (q, J=7.1 Hz, 2H), 1.67 (d, J=7.0 Hz, 3H), 1.29 (t, J=7.1 Hz, 3H), 0.22 (s, 9H) ppm.

### Step 2:

To a solution of 1,1,1-trifluoropropan-2-one (8 mL, 89.4 mmol) in DCM (80 mL) stirring at −78° C. was added TiCl₄ (70 mL of 1 M in DCM, 70.00 mmol) via cannula. To the resulting solution, a solution of ethyl (Z)-2-diazo-3-trimethylsilyloxypent-3-enoate (36.1 g of 31.3% w/w, 46.6 mmol) in 40 mL of DCM was added dropwise over 15 min. After 100 min the reaction was carefully quenched with water, allowing the temperature to rise slowly, and then extracted with DCM. The combined organic layers were dried (MgSO₄), filtered, and concentrated in vacuo. Purification by flash chromatography (330 g SiO₂, 0 to 20% EtOAc in heptane) gave ethyl rac-(4R,5S)-2-diazo-6,6,6-trifluoro-5-hydroxy-4,5-dimethyl-3-oxohexanoate (8.82 g, 67%), which was stored as a solution in toluene. ¹H NMR (500 MHZ, Chloroform-d) δ 4.33 (q, J=7.1 Hz, 2H), 4.14 (q, J=7.0 Hz, 1H), 3.98 (s, 1H), 1.43 (q, J=1.2 Hz, 3H), 1.35 (t, J=7.1 Hz, 3H), 1.31 (dq, J=7.0, 1.4 Hz, 3H) ppm. ESI-MS m/z calc. 282.08273, found 283.1 (M+1)⁺; 281.0 (M−1); Retention time: 0.76 minutes.

### Step 3:

A solution of dirhodium tetraacetate (245 mg, 0.55 mmol) in benzene (32 mL) was heated at reflux for 10 min before a solution of ethyl rac-(4R,5S)-2-diazo-6,6,6-trifluoro-5-hydroxy-4,5-dimethyl-3-oxohexanoate (10 g, 35.4 mmol) in benzene (13 mL) was added slowly via addition funnel while refluxing for 60 min. The mixture was then concentrated in vacuo to give ethyl rac-(4R,5R)-4,5-dimethyl-3-oxo-5-(trifluoromethyl)tetrahydrofuran-2-carboxylate (9.0 g, 100%) as a green coloured residue containing residual catalyst, and as a mixture of epimers at the position next to the ester. This material was used without further purification. ¹H NMR (500 MHZ, Chloroform-d) δ 4.83-4.57 (m, 1H), 4.38-4.16 (m, 2H), 2.60 (dddd, J=9.3, 8.2, 5.6, 1.4 Hz, 1H), 1.73-1.63 (m, 3H), 1.30 (t, J=7.1 Hz, 3H), 1.24 (ddq, J=6.4, 4.1, 1.9 Hz, 3H) ppm.

### Step 4:

To a stirred solution of ethyl rac-(4R,5R)-4,5-dimethyl-3-oxo-5-(trifluoromethyl)tetrahydrofuran-2-carboxylate (48 g, 188.83 mmol) in DCM (400 mL) at −78° C. was added DIPEA (29.680 g, 40 mL, 229.64 mmol). A solution of trifluoromethylsulfonyl trifluoromethanesulfonate (53.440 g, 32 mL, 189.41 mmol) in DCM (200 mL) at the same temperature was added to the reaction mixture over 1 h. The reaction mixture was stirred for 30 min at 0° C. before being quenched with 100 mL saturated aqueous NaHCO₃ solution. The organic layer was separated and aqueous layer extracted with DCM (160 mL). The combined organic layers were dried over MgSO₄ and concentrated in vacuo to give ethyl rac-(4R,5R)-4,5-dimethyl-5-(trifluoromethyl)-3-(((trifluoromethyl) sulfonyl)oxy)-4,5-dihydrofuran-2-carboxylate (71 g, 97%). ¹H NMR (400 MHZ, Chloroform-d) δ 4.38-4.32 (m, 2H), 3.29-3.23 (m, 1H), 1.64 (s, 3H), 1.37-1.33 (m, 6H) ppm.

### Step 5:

To stirred a solution of ethyl rac-(4R,5R)-4,5-dimethyl-5-(trifluoromethyl)-3-(((trifluoromethyl) sulfonyl)oxy)-4,5-dihydrofuran-2-carboxylate (26 g, 67.311 mmol) in toluene (130.00 mL) was added (3,4-difluoro-2-methoxyphenyl) boronic acid (14 g, 74.5 mmol) followed by K₃PO₄ (100 mL of 2 M, 200.00 mmol) under an argon atmosphere. The reaction was degassed before tetrakis(triphenylphosphine) palladium (0) (4 g, 3.46 mmol) was added. After further degassing, the reaction was heated at 100° C. for 2 h. The reaction was diluted in water and the aqueous layer extracted with EtOAc (2×100 mL). The combined organic layers were concentrated in vacuo. Purification by flash chromatography (SiO₂, 0 to 10% EtOAc in heptane) gave ethyl rac-3-(3,4-difluoro-2-methoxyphenyl)-4,5-dimethyl-5-(trifluoromethyl)-4,5-dihydrofuran-2-carboxylate (24.4 g, 93%) as a 6:1 diastereomeric mixture, with the major isomer believed to be ethyl rac-(4S,5R)-3-(3,4-difluoro-2-methoxyphenyl)-4,5-dimethyl-5-(trifluoromethyl)-4,5-dihydrofuran-2-carboxylate. Major isomer: ¹H NMR (400 MHZ, Chloroform-d) δ 6.88-6.79 (m, 2H), 4.17-4.09 (m, 2H), 3.90 (s, 3H), 3.46 (q, J=7.4 Hz, 1H), 1.67 (s, 3H), 1.12 (t, J=7.4 Hz, 3H), 1.06 (dd, J=5.4, 2.7 Hz, 3H) ppm. Minor isomer ¹H NMR (400 MHZ, Chloroform-d) δ 6.88-6.79 (m, 2H), 4.17-4.09 (m, 2H), 3.88 (s, 3H), 3.76-3.71 (m, 1H), 1.51 (s, 3H), 1.12 (t, J=7.4 Hz, 3H), 0.99 (dd, J=5.4, 2.7 Hz, 3H) ppm. ESI-MS m/z calc. 380.1047, found 381.02 (M+1)⁺; Retention time: 2.09 minutes.

### Step 6:

To an ice-cooled solution of ethyl rac-3-(3,4-difluoro-2-methoxyphenyl)-4,5-dimethyl-5-(trifluoromethyl)-4,5-dihydrofuran-2-carboxylate (110 g, 243.0 mmol) in DCM (360 mL) at 0° C. was added BBr₃ (370 mL of 1 M, 370.0 mmol) dropwise. Upon completion the mixture was quenched by addition of water and then aqueous sodium bicarbonate solution. The aqueous layer was extracted with DCM and the combined organic layers dried over MgSO₄ and concentrated in vacuo. The residue was dissolved in DCM (430 mL) at ambient temperature and TFA (40 mL, 519.2 mmol) was added, then the reaction was heated to 45° C. Upon completion, the mixture was quenched by addition of aqueous sodium bicarbonate solution and the aqueous layer extracted with DCM, dried over MgSO₄ and concentrated in vacuo to give the desired product in a 5:1 mixture of diastereomers. Recrystallization was carried out by solubilizing the crude in the smallest possible amount of DCM and adding a layer of heptane on top of this solution (liquid-liquid diffusion). After approx. 1 h, 56.5 g (d.r. 97:3 syn: anti) from the first and second crystallization was obtained, and a further 4.6 g (d.r. 96:4 syn: anti) from the third crystallization was obtained. The first to third recrystallization batches were combined to give rac-6,7-difluoro-1,2-dimethyl-2-(trifluoromethyl)-1,2-dihydro-4H-furo[2,3-c]chromen-4-one (61 g, 78%), with the major isomer believed to be rac-(1S,2R)-6,7-difluoro-1,2-dimethyl-2-(trifluoromethyl)-1,2-dihydro-4H-furo[2,3-c]chromen-4-one. ESI-MS m/z calc. 320.04718, found 321.5 (M+1)⁺; 319.6 (M−1); Retention time: 3.17 minutes.

### Step 7:

Rac-(1S,2R)-6,7-difluoro-1,2-dimethyl-2-(trifluoromethyl)-1,2-dihydro-4H-furo[2,3-c]chromen-4-one (30 g, 93.69 mmol) was dissolved in EtOAc (400 mL) and stirred with activated charcoal (6 g, 499.6 mmol) (0.2 g/g of substrate) at ambient temperature for 4 hours and 30 minutes. The mixture was filtered through a pad of celite and washed with EtOAc. The filtrate was concentrated in vacuo to give a white solid. The white solid was suspended in MeOH (600 mL) and added to a suspension of Pd(OH)₂ (13.62 g of 20% w/w, 19.40 mmol) in MeOH (150 mL) in a 2.25 L Parr bottle. The resulting mixture was shaken in the Parr hydrogenator under a hydrogen pressure of 60 psi overnight. The suspension was filtered through celite under a nitrogen atmosphere, rinsed with MeOH and then with EtOAc, and the resulting filtrate was concentrated in vacuo to give methyl rac-(2S,3S,4S,5R)-3-(3,4-difluoro-2-hydroxyphenyl)-4,5-dimethyl-5-(trifluoromethyl)tetrahydrofuran-2-carboxylate (32.75 g, 99%). ¹H NMR (400 MHZ, Methanol-d₄) δ 7.05 (ddq, J=9.4, 5.9, 1.9 Hz, 1H), 6.57 (ddd, J=10.0, 9.0, 7.6 Hz, 1H), 5.01 (d, J=6.0 Hz, 1H), 4.34 (dd, J=8.4, 6.0 Hz, 1H), 3.49 (s, 3H), 3.01-2.86 (m, 1H), 1.50 (q, J=1.2 Hz, 3H), 0.89 (dq, J=7.6, 1.9 Hz, 3H) ppm. ESI-MS m/z calc. 354.08905, found 353.3 (M−1); Retention time: 0.81 minutes.

### Step 8:

A solution of methyl rac-(2S,3S,4S,5R)-3-(3,4-difluoro-2-hydroxyphenyl)-4,5-dimethyl-5-(trifluoromethyl)tetrahydrofuran-2-carboxylate (60.8 g, 171.6 mmol) in THF (620 mL) was cooled to 1° C. Potassium tert-butoxide (65.0472 g, 579.7 mmol) was added over 10 min, keeping the internal temperature below 10° C. The mixture was stirred at 0° C. for a further 5 min, and then the mixture was warmed slightly. When the temperature had reached 13° C., the reaction was cooled down again with an ice bath before adding 2 M HCl (365 mL, to pH 1), keeping the internal temperature below 15° C. Water (300 mL) was added, the layers were separated, and the aqueous layer was extracted with EtOAc (110 mL). The combined organic extracts were washed with brine (300 mL), dried over MgSO₄, filtered, and concentrated in vacuo to give rac-(2R,3S,4S,5R)-3-(3,4-difluoro-2-hydroxyphenyl)-4,5-dimethyl-5-(trifluoromethyl)tetrahydrofuran-2-carboxylic acid (58.22 g, 100%). ¹H NMR (400 MHZ, Methanol-d₄) δ 7.00 (ddd, J=8.4, 5.6, 2.3 Hz, 1H), 6.69 (ddd, J=10.1, 8.8, 7.5 Hz, 1H), 4.98 (d, J=10.5 Hz, 1H), 4.18 (dd, J=10.5, 7.6 Hz, 1H), 2.83 (p, J=7.5 Hz, 1H), 1.59 (q, J=1.2 Hz, 3H), 0.76 (dq, J=7.2, 2.2 Hz, 3H) ppm. ESI-MS m/z calc. 340.0734, found 339.0 (M−1); Retention time: 0.47 minutes.

### Steps 9 and 10:

1-Bromo-2-methoxyethane (1.4 mL, 14.90 mmol) was added dropwise to a suspension of rac-(2R,3S,4S,5R)-3-(3,4-difluoro-2-hydroxyphenyl)-4,5-dimethyl-5-(trifluoromethyl)tetrahydrofuran-2-carboxylic acid (1 g, 2.939 mmol) and cesium carbonate (4.8 g, 14.73 mmol) in acetonitrile (50 mL). The reaction was stirred at 70° C. for 24 h before being filtered and concentrated in vacuo to give 2-methoxyethyl rac-(2R,3S,4S,5R)-3-(3,4-difluoro-2-(2-methoxyethoxy)phenyl)-4,5-dimethyl-5-(trifluoromethyl)tetrahydrofuran-2-carboxylate, which was used without further purification in the next step. ¹H NMR (400 MHZ, Chloroform-d) δ 7.04-6.76 (m, 2H), 5.06-4.87 (m, 1H), 4.46-4.15 (m, 5H), 3.73-3.61 (m, 3H), 3.52-3.43 (m, 2H), 3.42 (s, 3H), 3.27 (s, 3H), 2.85 (p, J=7.5 Hz, 1H), 1.65 (s, 1H), 0.76 (dp, J=6.9, 2.2 Hz, 4H) ppm. ESI-MS m/z calc. 456.15714, found 455.1 (M−1); Retention time: 1.02 minutes.

A 2 M LiOH solution (1.5 mL, 3.00 mmol) was added to a solution of 2-methoxyethyl rac-(2R,3S,4S,5R)-3-(3,4-difluoro-2-(2-methoxyethoxy)phenyl)-4,5-dimethyl-5-(trifluoromethyl)tetrahydrofuran-2-carboxylate in ethanol (30 mL). The mixture was stirred at ambient temperature overnight. The reaction mixture was acidified by the addition of 3 M HCl in MeOH (1 mL). The mixture was then filtered, and the mother liquors were concentrated in vacuo to give rac-(2R,3S,4S,5R)-3-(3,4-difluoro-2-(2-methoxyethoxy)phenyl)-4,5-dimethyl-5-(trifluoromethyl)tetrahydrofuran-2-carboxylic acid (1.1 g, 94%). ESI-MS m/z calc. 398.11526, found 399.1 (M+1)⁺; 397.1 (M−1); Retention time: 0.61 minutes.

### Steps 11, 12 and 13:

Oxalyl chloride (285 μL, 3.27 mmol) and DMF (5 μL, 0.065 mmol) were successively added to a solution of rac-(2R,3S,4S,5R)-3-(3,4-difluoro-2-(2-methoxyethoxy)phenyl)-4,5-dimethyl-5-(trifluoromethyl)tetrahydrofuran-2-carboxylic acid (260 mg, 0.65 mmol) in DCM (6.5 mL). The mixture was stirred at ambient temperature for 90 min. The mixture was then concentrated in vacuo. The residue was diluted in DCM (3 mL) and added dropwise over 5 min to a stirred solution of methyl 4-aminopicolinate (150 mg, 0.99 mmol), DMAP (4.63 mg, 0.038 mmol) and Et₃N (280 μL, 2.01 mmol) in DCM (5 mL) at ambient temperature. The reaction was stirred overnight. The mixture was then concentrated in vacuo. Purification by flash chromatography (12 g SiO₂, methanol) gave methyl rac-4-((2R,3S,4S,5R)-3-(3,4-difluoro-2-(2-methoxyethoxy)phenyl)-4,5-dimethyl-5-(trifluoromethyl)tetrahydrofuran-2-carboxamido) picolinate (150 mg), which was used directly in the next reaction.

Methyl rac-4-((2R,3S,4S,5R)-3-(3,4-difluoro-2-(2-methoxyethoxy)phenyl)-4,5-dimethyl-5-(trifluoromethyl)tetrahydrofuran-2-carboxamido) picolinate (150 mg) was dissolved in methanolic ammonia (5 mL of 7 M, 35.00 mmol) and stirred at ambient temperature for 24 h. The reaction mixture was then concentrated in vacuo. Purification by reverse phase preparative chromatography gave rac-4-((2R,3S,4S,5R)-3-(3,4-difluoro-2-(2-methoxyethoxy)phenyl)-4,5-dimethyl-5-(trifluoromethyl)tetrahydrofuran-2-carboxamido) picolinamide.

The enantiomers of rac-4-((2R,3S,4S,5R)-3-(3,4-difluoro-2-(2-methoxyethoxy)phenyl)-4,5-dimethyl-5-(trifluoromethyl)tetrahydrofuran-2-carboxamido) picolinamide were separated by chiral SFC using a Chiralpak AS-H column, 5 μm particle size, 25 cm×10 mm from Daicel Corporation (Mobile phase: 25% methanol: acetonitrile (in a 1:1 ratio, supplemented with 0.2% DMPA), 75% CO₂; System pressure: 100 bar) on a Minigram SFC instrument from Berger Instruments:

First Eluting Isomer (rt=4.93 min): rel-4-((2S,3R,4R,5S)-3-(3,4-difluoro-2-(2-methoxyethoxy)phenyl)-4,5-dimethyl-5-(trifluoromethyl)tetrahydrofuran-2-carboxamido) picolinamide (1, 18.2 mg, 10%). ¹H NMR (400 MHZ, Chloroform-d) δ 8.90 (s, 1H), 8.46 (d, J=5.5 Hz, 1H), 8.20 (dd, J=5.5, 2.2 Hz, 1H), 8.05-7.95 (m, 1H), 7.88 (s, 1H), 7.13 (ddd, J=8.1, 5.5, 2.1 Hz, 1H), 6.95 (td, J=9.2, 7.5 Hz, 1H), 5.82 (d, J=4.2 Hz, 1H), 5.04 (d, J=11.5 Hz, 1H), 4.41-4.17 (m, 3H), 3.71-3.55 (m, 2H), 3.30 (s, 3H), 2.89 (p, J=7.5 Hz, 1H), 1.73 (s, 3H), 0.81 (dt, J=7.4, 2.4 Hz, 3H) ppm. ESI-MS m/z calc. 517.16364, found 518.4 (M+1)⁺; 516.5 (M−1); Retention time: 3.25 minutes.

Second Eluting Isomer (rt=5.37 min): rel-4-((2R,3S,4S,5R)-3-(3,4-difluoro-2-(2-methoxyethoxy)phenyl)-4,5-dimethyl-5-(trifluoromethyl)tetrahydrofuran-2-carboxamido) picolinamide (2, 18.6 mg, 11%). ¹H NMR (400 MHZ, Chloroform-d) δ 8.88 (s, 1H), 8.46 (d, J=5.6 Hz, 1H), 8.19 (dd, J=5.6, 2.2 Hz, 1H), 8.01 (dd, J=2.2, 0.6 Hz, 1H), 7.86 (d, J=4.6 Hz, 1H), 7.13 (ddd, J=8.1, 5.5, 2.1 Hz, 1H), 6.95 (td, J=9.3, 7.5 Hz, 1H), 5.82 (d, J=4.2 Hz, 1H), 5.04 (d, J=11.5 Hz, 1H), 4.41-4.17 (m, 3H), 3.71-3.55 (m, 2H), 3.30 (s, 3H), 2.89 (p, J=7.5 Hz, 1H), 1.73 (s, 3H), 0.81 (dt, J=7.6, 2.3 Hz, 3H) ppm. ESI-MS m/z calc. 517.16364, found 518.5 (M+1)⁺; 516.5 (M−1); Retention time: 3.20 minutes.

The following compounds were made using the method described in Example 1, except that the Suzuki coupling step 5 was carried out at 50° C. over 30 min, using PdCl₂(PPh₃)₂ as the catalyst together with (4-fluoro-2-methoxy-3-methylphenyl) boronic acid (Intermediate G) as the boronic acid, NaHCO₃ as the base and a mixture of water and 1,4-dioxane as the solvent. In step 9, K₂CO₃ was used as the base in place of Cs₂CO₃. In step 10, MeOH was used as the solvent in place of EtOH. The conditions used for the amide coupling step 11 where those described in Example 6 step 4, using methyl 4-aminopyrimidine-2-carboxylate as the coupling partner. In step 13, the enantiomers were separated by chiral SFC using a Chiralcel OD-H column, 5 mm particle size, 25 cm×10 mm from Daicel Corporation (Mobile phase: 30% methanol (supplemented with 20 mM NH₃), 70% CO₂; System pressure: 100 bar) on a Minigram SFC instrument from Berger Instruments:

| Cmpd No. | Compound Name | LC/MS | NMR (shifts in ppm) |
|---|---|---|---|
| 3 | rel-4-((2R,3S,4S,5R)-3-(4- | ESI-MS m/z calc. | ¹H NMR (400 MHz, |
| | fluoro-2-(2-methoxyethoxy)-3- | 514.18396, found 515.2 | Methanol-d₄) δ 8.77 (s, |
| | methylphenyl)-4,5-dimethyl-5- | (M + 1)⁺; 513.2 (M − 1)⁻; | 1H), 8.24 (s, 1H), 7.27 |
| | (trifluoromethyl)tetrahydrofuran- | Retention time: 3.15 | (dd, J = 8.7, 6.3 Hz, 1H), |
| | 2-carboxamido)pyrimidine-2- | minutes | 6.88 (t, J = 8.9 Hz, 1H), |
| | carboxamide | | 5.12 (d, J = 11.2 Hz, 1H), |
| | (First eluting isomer by SFC on | | 4.51 (dd, J = 11.2, 7.5 Hz, |
| | Chiralcel OD-H column, rt = | | 1H), 4.06 (ddd, J = 10.8, |
| | 2.64 min) | | 5.4, 2.2 Hz, 1H), 3.85 |
| | | | (ddd, J = 10.7, 7.1, 2.2 |
| | | | Hz, 1H), 3.71 (ddd, J = |
| | | | 11.2, 7.1, 2.2 Hz, 1H), |
| | | | 3.59 (ddd, J = 11.2, 5.4, |
| | | | 2.2 Hz, 1H), 3.34 (s, 3H), |
| | | | 2.81 (h, J = 7.1, 6.7 Hz, |
| | | | 1H), 2.21 (d, J = 2.2 Hz, |
| | | | 3H), 1.73 (d, J = 1.3 Hz, |
| | | | 3H), 0.79 (dd, J = 7.6, 2.4 |
| | | | Hz, 3H) ppm; amide NH |
| | | | and NH2 not observed. |
| 4 | rel-4-((2S,3R,4R,5S)-3-(4- | ESI-MS m/z calc. | ¹H NMR (400 MHz, |
| | fluoro-2-(2-methoxyethoxy)-3- | 514.18393, found 515.2 | Methanol-d₄) δ 8.78 (s, |
| | methylphenyl)-4,5-dimethyl-5- | (M + 1)⁺; 513.2 (M − 1)⁻; | 1H), 8.24 (s, 1H), 7.27 |
| | (trifluoromethyl)tetrahydrofuran- | Retention time: 3.15 | (dd, J = 8.7, 6.3 Hz, 1H), |
| | 2-carboxamido)pyrimidine-2- | minutes | 6.88 (t, J = 8.9 Hz, 1H), |
| | carboxamide | | 5.12 (d, J = 11.2 Hz, 1H), |
| | (Second eluting isomer by SFC | | 4.50 (dd, J = 11.2, 7.5 Hz, |
| | on Chiralcel OD-H column, rt = | | 1H), 4.06 (ddd, J = 10.8, |
| | 3.24 min) | | 5.4, 2.2 Hz, 1H), 3.85 |
| | | | (ddd, J = 10.8, 7.1, 2.2 |
| | | | Hz, 1H), 3.71 (ddd, J = |
| | | | 11.2, 7.0, 2.2 Hz, 1H), |
| | | | 3.59 (ddd, J = 11.2, 5.4, |
| | | | 2.2 Hz, 1H), 3.34 (s, 3H), |
| | | | 2.82 (p, J = 7.3 Hz, 1H), |
| | | | 2.21 (d, J = 2.2 Hz, 3H), |
| | | | 1.73 (d, J = 1.3 Hz, 3H), |
| | | | 0.86-0.76 (m, 3H) ppm; |
| | | | amide NH and NH₂ not |
| | | | observed. |

### Example 2

### 4-((2R,3S,4S,5R)-3-(3,4-difluoro-2-(2-hydroxyethoxy)phenyl)-4,5-dimethyl-5-(trifluoromethyl)tetrahydrofuran-2-carboxamido) picolinamide (5)

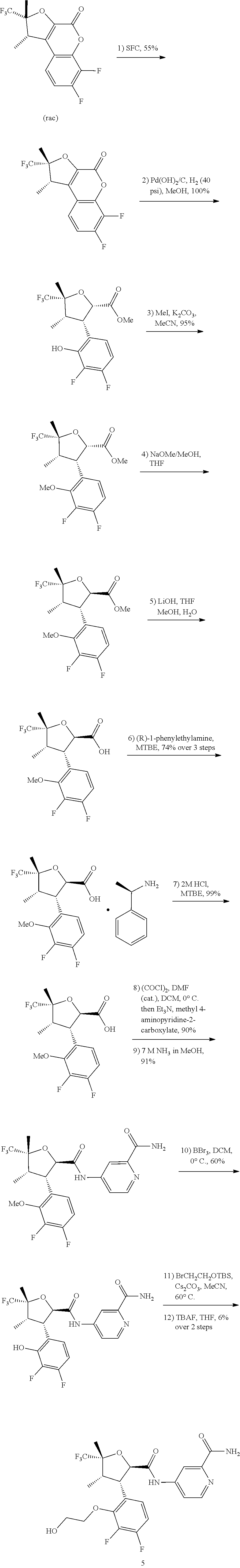

### Step 1:

rac-(1S,2R)-6,7-Difluoro-1,2-dimethyl-2-(trifluoromethyl)-1,2-dihydro-4H-furo[2,3-c]chromen-4-one (Product of Step 6, Example 1, 1348 g, 4.366 mol) was separated by chiral SFC using a (R,R)-Whelk-O1 column, 5 μm particle size, 15 cm×3 cm from Regis Technologies (Mobile phase: 10% isopropanol, 90% CO₂; Flow rate: 5 mL/min; System pressure: 100 bar; Column temperature: 35° C.) on a MultiGram III SFC instrument from Berger Instruments to give:

First Eluting Isomer (rt=1.85 min): (1R,2S)-6,7-difluoro-1,2-dimethyl-2-(trifluoromethyl)-1,2-dihydro-4H-furo[2,3-c]chromen-4-one). ¹H NMR (400 MHZ, DMSO-d₆) δ 7.57 (ddd, J=9.0, 5.5, 2.0 Hz, 1H), 7.51 (ddd, J=10.3, 9.0, 7.0 Hz, 1H), 4.03 (q, J=7.2 Hz, 1H), 1.65 (s, 3H), 1.45 (dt, J=6.9, 2.2 Hz, 3H) ppm. ESI-MS m/z calc. 320.04718, found 321.3 (M+1)⁺; 319.4 (M−1); Retention time: 3.19 minutes.

Second Eluting Isomer (rt=2.38 min): (1S,2R)-6,7-Difluoro-1,2-dimethyl-2-(trifluoromethyl)-1,2-dihydro-4H-furo[2,3-c]chromen-4-one (366.99 g, 26%). ¹H NMR (400 MHZ, DMSO-d₆) δ 7.57 (ddd, J=9.0, 5.5, 2.0 Hz, 1H), 7.50 (ddd, J=10.3, 9.0, 7.0 Hz, 1H), 4.03 (q, J=7.2 Hz, 1H), 1.65 (s, 3H), 1.45 (dt, J=6.9, 2.2 Hz, 3H) ppm. ESI-MS m/z calc. 320.04518, found 321.4 (M+1)⁺; 319.4 (M−1); Retention time: 3.20 minutes.

### Step 2:

A solution of (1S,2R)-6,7-difluoro-1,2-dimethyl-2-(trifluoromethyl)-1,2-dihydro-4H-furo[2,3-c]chromen-4-one (0.89 kg, 2.78 mol) and 20% palladium hydroxide on carbon (50% wet, 0.39 kg, 0.278 mol) in MeOH (12 L) was stirred under a 40 psi pressure of hydrogen overnight. An increase in the reaction temperature to 37° C. was observed after reacting overnight and the mixture was cooled to 24° C. The hydrogenation was continued for a total of 48 h. The mixture was filtered through celite, washed with MeOH (20 L), and the filtrates were concentrated in vacuo. The residue was dissolved in toluene (4 L) and concentrated in vacuo, and this process repeated. The residue was dried under vacuum at 40° C. overnight to give methyl(2S,3S,4S,5R)-3-(3,4-difluoro-2-hydroxyphenyl)-4,5-dimethyl-5-(trifluoromethyl)tetrahydrofuran-2-carboxylate (1.0 kg at 91% purity, 100%) as a beige solid. ¹H NMR (400 MHZ, DMSO-d₆) 10.20 (br s, 1H), 6.94 (br t, J=7.4 Hz, 1H), 6.79-6.69 (m, 1H), 5.10 (d, J=6.0 Hz, 1H), 4.20 (dd, J=6.1, 8.2 Hz, 1H), 3.43 (s, 3H), 2.94 (quin, J=7.7 Hz, 1H), 1.46 (s, 3H), 0.77 (br d, J=6.8 Hz, 3H) ppm.

### Step 3:

Potassium carbonate (2.0 kg, 14.4 mol) and iodomethane (800 mL, 12.8 mol) were sequentially added to a solution of methyl(2S,3S,4S,5R)-3-(3,4-difluoro-2-hydroxyphenyl)-4,5-dimethyl-5-(trifluoromethyl)tetrahydrofuran-2-carboxylate (1.0 kg, 2.82 mol) in acetonitrile (10 L) under nitrogen stirring at ambient temperature. After stirring overnight, additional iodomethane (120 mL, 2 mmol) was added. After stirring overnight again, additional iodomethane (60 mL, 0.85 mmol) was added and the mixture was stirred for a further 3 days. The reaction mixture was diluted with MTBE (30 L), treated with celite (1 kg) and filtered through a bed of celite (1 kg) washing with MTBE (10 L). The filtrate was filtered a second time through celite (1 kg), washed with MTBE (4 L), and the filtrate concentrated in vacuo. The residue was dissolved in toluene (4 L) and concentrated in vacuo, and this process repeated. The residue was dried under vacuum at 40° C. overnight to give methyl(2S,3S,4S,5R)-3-(3,4-difluoro-2-methoxyphenyl)-4,5-dimethyl-5-(trifluoromethyl)tetrahydrofuran-2-carboxylate (0.99 kg at 90% purity, 95%) as a brown solid. ¹H NMR (400 MHZ, DMSO-d₆) 7.14-7.00 (m, 2H), 5.14 (d, J=6.0 Hz, 1H), 4.15 (dd, J=6.2, 8.4 Hz, 1H), 3.88 (d, J=1.7 Hz, 3H), 2.97 (quin, J=7.8 Hz, 1H), 1.48 (s, 3H), 0.72 (br d, J=6.6 Hz, 3H) ppm.

### Step 4 and 5:

Sodium methoxide (25% in methanol, 65 mL, 0.28 mol) was added to a solution of methyl(2S,3S,4S,5R)-3-(3,4-difluoro-2-methoxyphenyl)-4,5-dimethyl-5-(trifluoromethyl)tetrahydrofuran-2-carboxylate (0.98 kg, 2.66 mol) in THF (10 L) and stirred at ambient temperature under nitrogen. After 5 h, MeOH (1 L), water (1 L) and lithium hydroxide monohydrate (0.168 kg, 4.0 mol) were sequentially added and the mixture was stirred overnight. The reaction mixture was poured into 1M HCl (4.4 L, 4.4 mol) then extracted with MTBE (20 L). The aqueous layer was further extracted with MTBE (2×5 L) and the combined organic layers washed with brine (2 L), dried over Na₂SO₄, filtered, and then treated with activated carbon (50 g, 5% w/w) with stirring for 1 h. The mixture was filtered through celite, washing with MTBE (2×4 L), and the filtrate concentrated in vacuo. The residue was dissolved in toluene (4 L) and concentrated in vacuo, then dissolved in MTBE (4 L) and concentrated in vacuo again to give (2R,3S,4S,5R)-3-(3,4-difluoro-2-methoxyphenyl)-4,5-dimethyl-5-(trifluoromethyl)tetrahydrofuran-2-carboxylic acid (1.06 kg at 77.7% purity) as an amber oil, which was used without further purification.

### Step 6:

Crude (2R,3S,4S,5R)-3-(3,4-difluoro-2-methoxyphenyl)-4,5-dimethyl-5-(trifluoromethyl)tetrahydrofuran-2-carboxylic acid (2.09 kg at 77% purity, 4.54 mol) was dissolved in MTBE (25 L) in a 100 L Chemglass reactor then stirred at 84 rpm at ambient temperature. A mixture of (R)-1-phenylethylamine (0.704 kg, 5.81 mol) and MTBE (2 L) was added to the reactor, followed by additional MTBE to give a total volume of 30 L in the reactor. After 2 h additional MTBE (2 L) was added to the reaction. After a total of 3.5 h the mixture was filtered, washing with MTBE (2 L). The reactor was rinsed with MTBE (4 L), which was used to rinse the solids, which were then compressed and dried on the Büchner funnel for 2 h. The solid product cake was loosened then dried under a stream of nitrogen and under vacuum overnight on the Büchner funnel. The isolated solids were dried in a convection oven at 40° C. for 24 h to give (2R,3S,4S,5R)-3-(3,4-difluoro-2-methoxyphenyl)-4,5-dimethyl-5-(trifluoromethyl)tetrahydrofuran-2-carboxylic acid (R)-1-phenylethan-1-amine salt (1.86 kg at 95.7% purity, 74% over 3 steps) as an off-white solid. ¹H NMR, 400 MHZ, DMSO-d₆) 8.34 (br s, 2H), 7.46-7.41 (m, 2H), 7.36-7.27 (m, 3H), 7.16-7.11 (m, 1H), 7.10-7.03 (m, 1H), 4.58 (d, J=9.9 Hz, 1H), 4.23 (q, J=6.7 Hz, 1H), 3.99 (dd, J=7.8, 9.8 Hz, 1H), 3.90 (d, J=2.0 Hz, 3H), 2.60 (quin, J=7.5 Hz, 1H), 1.50 (s, 3H), 1.40 (d, J=6.7 Hz, 3H), 0.71-0.59 (m, 3H) ppm.

### Step 7:

To a suspension of (2R,3S,4S,5R)-3-(3,4-difluoro-2-methoxyphenyl)-4,5-dimethyl-5-(trifluoromethyl)tetrahydrofuran-2-carboxylic acid (1R)-1-phenylethanamine salt (10.6 g, 22.29 mmol) in MTBE (250 mL) was added HCl (200 mL of 2 M, 400.0 mmol). The layers were separated and the organic layer was washed with water (200 mL), dried over MgSO₄, filtered, and concentrated in vacuo to give (2R,3S,4S,5R)-3-(3,4-difluoro-2-methoxyphenyl)-4,5-dimethyl-5-(trifluoromethyl)tetrahydrofuran-2-carboxylic acid (8.4 g, 99%) as an oil. ¹H NMR (400 MHZ, Chloroform-d) δ 6.96 (ddd, J=7.9, 5.6, 2.0 Hz, 1H), 6.88 (td, J=9.2, 7.3 Hz, 1H), 4.96 (d, J=10.5 Hz, 1H), 4.15 (dd, J=10.5, 8.0 Hz, 1H), 4.02 (d, J=2.8 Hz, 3H), 2.74 (p, J=7.6 Hz, 1H), 1.64 (t, J=1.2 Hz, 3H), 0.79 (dq, J=7.4, 2.3 Hz, 3H) ppm.

### Step 8:

Oxalyl chloride (2.2 mL, 25.22 mmol) was added to a stirred solution of (2R,3S,4S,5R)-3-(3,4-difluoro-2-methoxy-phenyl)-4,5-dimethyl-5-(trifluoromethyl)tetrahydrofuran-2-carboxylic acid (8 g, 16.83 mmol) and DMF (13 μL, 0.1679 mmol) in DCM (60 mL). The reaction mixture was stirred for 2 h. The reaction mixture was concentrated in vacuo. The residue, dissolved in DCM (40 mL), was added to a solution of methyl 4-aminopyridine-2-carboxylate (2.8 g, 18.40 mmol) and triethylamine (2.8 mL, 20.09 mmol) at 0° C. The resulting mixture was stirred at room temperature for 5 h. The reaction mixture was quenched with a mixture of water (48 mL) and 1 M citric acid (24 mL, 24.00 mmol). The layers were separated and the organic phase was dried over MgSO₄, filtered, and concentrated in vacuo to give methyl 4-((2R,3S,4S,5R)-3-(3,4-difluoro-2-methoxyphenyl)-4,5-dimethyl-5-(trifluoromethyl)tetrahydrofuran-2-carboxamido) picolinate (8.38 g, 90%). ¹H NMR (400 MHZ, DMSO-d₆) δ 10.77 (s, 1H), 8.57 (dd, J=5.4, 0.6 Hz, 1H), 8.36 (dd, J=2.2, 0.7 Hz, 1H), 7.85 (dd, J=5.5, 2.2 Hz, 1H), 7.25-7.07 (m, 2H), 5.11 (d, J=10.2 Hz, 1H), 4.25 (dd, J=10.2, 7.6 Hz, 1H), 3.94 (d, J=2.1 Hz, 3H), 3.86 (s, 3H), 2.77 (p, J=7.5 Hz, 1H), 1.60 (s, 3H), 0.78-0.64 (m, 3H) ppm. ESI-MS m/z calc. 488.13705, found 489.6 (M+1)⁺; 487.5 (M-1); Retention time: 3.38 minutes.

### Step 9:

A solution of methyl 4-((2R,3S,4S,5R)-3-(3,4-difluoro-2-methoxyphenyl)-4,5-dimethyl-5-(trifluoromethyl)tetrahydrofuran-2-carboxamido) picolinate (7.1 g, 14.54 mmol) in methanolic ammonia (70 mL of 7 M, 490.0 mmol) was stirred at room temperature for 19 h. The mixture was concentrated in vacuo to give a pale orange solid. EtOAc (30 mL) was added and the resulting slurry was heated at 60° C. The solution was cooled down to 50° C. and heptane (17 mL) was slowly added through an addition funnel. At the end of addition, the hazy solution was left to stand. A pale orange solid was filtered to give a first crop (5.091 g). The filtrates were concentrated to a third of the volume and the orange precipitate was filtered out to give a second crop (1.14 g). The crops were combined to give 4-((2R,3S,4S,5R)-3-(3,4-difluoro-2-methoxyphenyl)-4,5-dimethyl-5-(trifluoromethyl)tetrahydrofuran-2-carboxamido) picolinamide (6.23 g, 91%). ¹H NMR (400 MHZ, DMSO-d₆) δ 10.72 (s, 1H), 8.50 (d, J=5.6 Hz, 1H), 8.29 (d, J=2.1 Hz, 1H), 8.06 (d, J=2.8 Hz, 1H), 7.84 (dd, J=5.5, 2.2 Hz, 1H), 7.62 (d, J=2.8 Hz, 1H), 7.24-7.11 (m, 2H), 5.11 (d, J=10.2 Hz, 1H), 4.26 (dd, J=10.2, 7.7 Hz, 1H), 3.95 (d, J=2.2 Hz, 3H), 2.78 (p, J=7.5 Hz, 1H), 1.62 (s, 3H), 0.73 (dt, J=7.3, 2.4 Hz, 3H) ppm. ESI-MS m/z calc. 473.1374, found 474.1 (M+1)⁺; Retention time: 0.92 minutes.

### Step 10:

BBr₃ (830.0 μL, 1 M in DCM, 0.83 mmol) was added at 0° C. to a stirred solution of 4-((2R,3S,4S,5R)-3-(3,4-difluoro-2-methoxyphenyl)-4,5-dimethyl-5-(trifluoromethyl)tetrahydrofuran-2-carboxamido) picolinamide (280 mg, 0.59 mmol) in DCM (6 mL). The reaction mixture was warmed slowly to ambient temperature and stirred for 24 h. The mixture was cooled down to 0° C. and further BBr₃ (800 μL, 1 M in DCM, 0.80 mmol) was added. The reaction was stirred at ambient temperature for 16 h. The reaction mixture was quenched by addition of water and a saturated aqueous sodium bicarbonate solution. The mixture was left to stir for 30 min, and the layers were then separated. The aqueous layer was extracted with DCM and the combined organic layers were dried, filtered, and concentrated in vacuo. Purification by flash chromatography (12 g SiO₂, 0 to 70% EtOAc in heptane) gave 4-((2R,3S,4S,5R)-3-(3,4-difluoro-2-hydroxyphenyl)-4,5-dimethyl-5-(trifluoromethyl)tetrahydrofuran-2-carboxamido) picolinamide (162 mg, 60%). ¹H NMR (500 MHZ, DMSO-d₆) δ 10.73 (s, 1H), 10.46 (s, 1H), 8.50 (d, J=5.6 Hz, 1H), 8.27 (s, 1H), 8.06 (s, 1H), 7.86-7.82 (m, 1H), 7.61 (s, 1H), 7.06-7.00 (m, 1H), 6.89-6.83 (m, 1H), 5.15-5.08 (m, 1H), 4.29-4.22 (m, 1H), 2.86-2.80 (m, 1H), 1.61 (s, 3H), 0.72 (d, J=7.2 Hz, 3H) ppm. ESI-MS m/z calc. 459.12173, found 460.7 (M+1)⁺; 458.8 (M−1); Retention time: 2.59 minutes.

### Step 11 and 12:

(2-Bromoethoxy) (tert-butyl)dimethylsilane (20 μL, 0.093 mmol) was added dropwise to a suspension of 4-((2R,3S,4S,5R)-3-(3,4-difluoro-2-hydroxyphenyl)-4,5-dimethyl-5-(trifluoromethyl)tetrahydrofuran-2-carboxamido) picolinamide (20 mg, 0.045 mmol) and cesium carbonate (50 mg, 0.15 mmol) in acetonitrile (6 mL). The reaction mixture was heated to 61° C. overnight. The mixture was concentrated in vacuo. TBAF (1 mL, 1M in THF) was added to a solution of the residue dissolved in THF. The mixture was stirred for 1 h, and the mixture was then concentrated in vacuo. Purification by reverse phase preparative chromatography gave 4-((2R,3S,4S,5R)-3-(3,4-difluoro-2-(2-hydroxyethoxy)phenyl)-4,5-dimethyl-5-(trifluoromethyl)tetrahydrofuran-2-carboxamido) picolinamide (5, 1.4 mg, 6%). ¹H NMR (400 MHZ, Chloroform-d) δ 8.86 (s, 1H), 8.37 (d, J=5.5 Hz, 1H), 8.04 (dd, J=5.5, 2.3 Hz, 1H), 7.90 (td, J=2.1, 0.6 Hz, 1H), 7.03 (ddd, J=8.1, 5.5, 2.1 Hz, 1H), 6.86 (td, J=9.2, 7.5 Hz, 1H), 4.96 (d, J=10.9 Hz, 1H), 4.34 (dd, J=10.9, 8.1 Hz, 1H), 4.29-4.05 (m, 1H), 3.80 (s, 2H), 2.75 (p, J=7.7 Hz, 1H), 2.34 (s, 1H), 1.66-1.55 (m, 3H), 0.73 (dt, J=7.6, 2.3 Hz, 3H) ppm. ESI-MS m/z calc. 503.14795, found 504.1 (M+1)⁺; 502.1 (M−1); Retention time: 0.81 minutes.

The following compounds were made using the method described in Example 2, except that step 11 was carried out using 5-(chloromethyl) oxazole as the alkylating agent and K₂CO₃ in place of Cs₂CO₃ as the base. Step 12 was omitted:

| Cmpd No. | Compound Name | LC/MS | NMR (shifts in ppm) |
|---|---|---|---|
| 6 | 4-((2R,3S,4S,5R)-3-(3,4- | ESI-MS m/z calc. | ¹H NMR (500 MHz, |
| | difluoro-2-(oxazol-5- | 540.1432, found 542.2 | DMSO-d₆) δ 10.69 (s, |
| | ylmethoxy)phenyl)-4,5- | (M + 1)⁺; 539.2 (M − 1)⁻; | 1H), 8.49 (d, J = 5.5 Hz, |
| | dimethyl-5- | Retention time: 3.10 | 1H), 8.43 (s, 1H), 8.26 (d, |
| | (trifluoromethyl)tetrahydrofuran- | minutes. | J = 2.2 Hz, 1H), 8.06 (d, J = |
| | 2-carboxamido)picolinamide | | 2.9 Hz, 1H), 7.84 (dd, J = |
| | | | 5.5, 2.2 Hz, 1H), 7.61 |
| | | | (d, J = 2.9 Hz, 1H), 7.34 |
| | | | (s, 1H), 7.29-7.08 (m, |
| | | | 2H), 5.37-5.20 (m, 2H), |
| | | | 5.09 (d, J = 10.6 Hz, 1H), |
| | | | 4.18 (dd, J = 10.6, 7.4 Hz, |
| | | | 1H), 2.60 (q, J = 7.4 Hz, |
| | | | 1H), 1.54 (s, 3H), 0.78- |
| | | | 0.59 (m, 3H) ppm. |

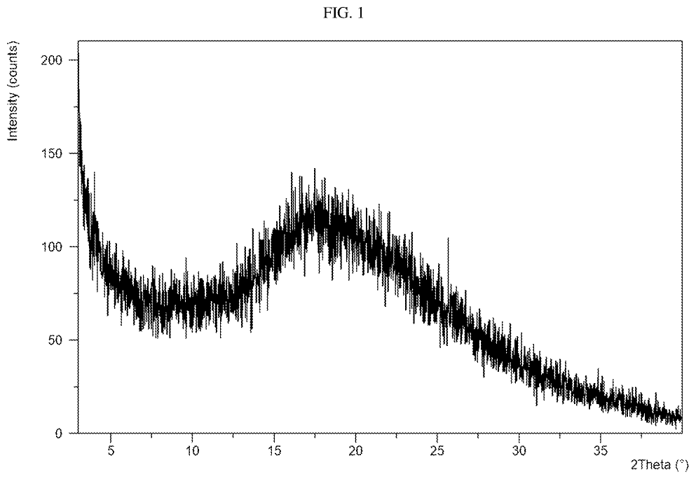

Compound 6 was analyzed by X-ray powder diffraction and determined to be amorphous (see FIG. 1).

The following compound was made using the method described in Example 2, except that step 11 was carried out at 80° C. for 6 h using (1s,3s)-3-(bromomethyl)-1-(trifluoromethyl)cyclobutan-1-ol as the alkylating agent, K₂CO₃ as the base and DMF as the solvent. Step 12 was omitted:

| Cmpd No. | Compound Name | LC/MS | NMR (shifts in ppm) |
|---|---|---|---|
| 7 | 4-((2R,3S,4S,5R)-3-(3,4- | ESI-MS m/z calc. | ¹H NMR (500 MHz, |
| | difluoro-2-(((1s,3R)-3-hydroxy- | 611.1666, found 612.6 | Methanol-d₄) δ 8.50 (d, |
| | 3- | (M + 1)⁺; 610.5 (M − 1)⁻; | 1H), 8.27 (d, 1H), 7.91 |
| | (trifluoromethyl)cyclobutyl)meth- | Retention time: 3.50 | (dd, 1H), 7.19-7.13 (m, |
| | oxy)phenyl)-4,5-dimethyl-5- | minutes. | 1H), 7.04-6.97 (md, 1H), |
| | (trifluoromethyl)tetrahydrofuran- | | 5.10-5.08 (d, 1H), 4.39- |
| | 2-carboxamido)picolinamide | | 4.34 (m, 1H), 4.31-4.28 |
| | | | (m, 1H), 4.17-4.13 (m, |
| | | | 1H), 3.76-3.74 (m, 1H), |
| | | | 2.86-2.79 (m, 1H), 2.64- |
| | | | 2.53 (m, 2H), 2.18-2.11 |
| | | | (m, 2H), 1.68 (s, 3H), |
| | | | 0.84-0.82 (m, 3H) ppm; |
| | | | amides NH and NH₂ and |
| | | | alcohol OH not observed. |

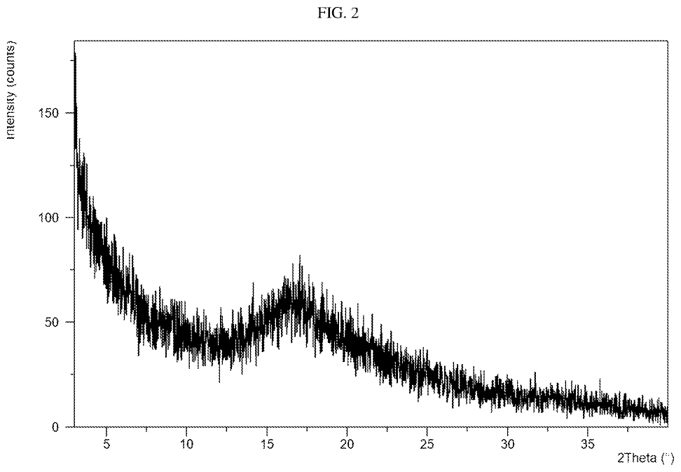

Compound 7 was analyzed by X-ray powder diffraction and determined to be amorphous (see FIG. 2).

The following compounds were made using the method described in Example 2, except that different amines were used in place of methyl 4-aminopyridine-2-carboxylate in Step 8. Steps 9 to 12 were omitted:

| Cmpd No. | Compound Name | LC/MS | NMR (shifts in ppm) |
|---|---|---|---|
| 8 | (2R,3S,4S,5R)-3-(3,4-difluoro- | ESI-MS m/z calc. | ¹H NMR (500 MHz, |
| | 2-methoxy-phenyl)-4,5- | 556.525, found 557.0 | DMSO-d₆) δ 10.66 (s, |
| | dimethyl-N-[2-(4- | (M + 1)⁺; Retention time: | 1H), 8.44 (d, J = 5.6 Hz, |
| | methylpiperazine-1-carbonyl)- | 3.15 minutes. | 1H), 7.81 (d, J = 2.1 Hz, |
| | 4-pyridyl]-5- | | 1H), 7.65 (dd, J = 5.6, 2.1 |
| | (trifluoromethyl)tetrahydrofuran- | | Hz, 1H), 7.23-7.09 (m, |
| | 2-carboxamide | | 2H), 5.10 (d, J = 10.2 Hz, |
| | | | 1H), 4.25 (dd, J = 10.2, |
| | | | 7.7 Hz, 1H), 3.95 (d, J = |
| | | | 2.0 Hz, 3H), 3.67-3.56 |
| | | | (m, 2H), 3.38-3.35 (m, |
| | | | 2H), 2.77 (p, J = 7.6 Hz, |
| | | | 1H), 2.35 (t, J = 5.2 Hz, |
| | | | 2H), 2.24 (t, J = 5.0 Hz, |
| | | | 2H), 2.18 (s, 3H), 1.60 (s, |
| | | | 3H), 0.76-0.67 (m, 3H) |
| | | | ppm. |
| 9 | (2R,3S,4S,5R)-3-(3,4-difluoro- | ESI-MS m/z calc. | ¹H NMR (400 MHz, |
| | 2-methoxyphenyl)-N-(5-fluoro- | 574.515, found 575.24 | Chloroform-d) δ 8.86 (s, |
| | 2-(4-methylpiperazine-1- | (M + 1)⁺; Retention time: | 1H), 8.59 (d, J = 6.4 Hz, |
| | carbonyl)pyridin-4-yl)-4,5- | 2.56 minutes | 1H), 8.40 (d, J = 1.8 Hz, |
| | dimethyl-5- | | 1H), 7.08-7.05 (m, 1H), |
| | (trifluoromethyl)tetrahydrofuran- | | 6.93-6.86 (m, 1H), 5.03 |
| | 2-carboxamide | | (d, J = 11.0 Hz, 1H), 4.06 |
| | | | (dd, J = 11.0, 7.8 Hz, |
| | | | 1H), 4.00 (d, J = 2.7 Hz, |
| | | | 3H), 3.83 ( br s, 2H), 3.58 |
| | | | (br s, 2H), 2.79-2.71 (m, |
| | | | 1H), 2.53 (d, J = 38.9 Hz, |
| | | | 4H), 2.37 (s, 3H), 1.67 (s, |
| | | | 3H), 0.80-0.77 (m, 3H) |
| | | | ppm. |
| 10 | (2R,3S,4S,5R)-3-(3,4-difluoro- | ESI-MS m/z calc. | ¹H NMR (400 MHz, |
| | 2-methoxyphenyl)-4,5- | 556.525, found 557.25 | Chloroform-d) δ 8.84 (s, |
| | dimethyl-N-(6-(4- | (M + 1)⁺; Retention time: | 1H), 8.21 (d, J = 8.2 Hz, |
| | methylpiperazine-1- | 2.56 minutes | 1H), 7.79 (t, J = 8.0 Hz, |
| | carbonyl)pyridin-2-yl)-5- | | 1H), 7.37 (d, J = 7.3 Hz, |
| | (trifluoromethyl)tetrahydrofuran- | | 1H), 7.09 (t, J = 6.4 Hz, |
| | 2-carboxamide | | 1H), 6.90 (dd, J = 16.5, |
| | | | 9.2 Hz, 1H), 5.01 (d, J = |
| | | | 11.0 Hz, 1H), 4.09 (q, |
| | | | 1H), 4.00 (d, J = 2.75 Hz, |
| | | | 3H), 4.00-3.99 (m, 2H), |
| | | | 3.10-2.84 (m, 2H), 2.78- |
| | | | 2.72 (m, 1H), 2.69 (s, |
| | | | 3H), 1.68 (s, 3H), 1.68- |
| | | | 1.46 (m, 4H), 0.79 (d, J = |
| | | | 5.5 Hz, 3H) ppm. |
| 11 | (2R,3S,4S,5R)-3-(3,4-difluoro- | ESI-MS m/z calc. | ¹H NMR (400 MHz, |
| | 2-methoxyphenyl)-4,5- | 557.513, found 558.22 | Chloroform-d) δ 9.05 (s, |
| | dimethyl-N-(2-(4- | (M + 1)⁺; Retention time: | 1H), 8.62 (d, J = 5.5 Hz, |
| | methylpiperazine-1- | 2.43 minutes. | 1H), 8.10 (d, J = 5.5 Hz, |
| | carbonyl)pyrimidin-4-yl)-5- | | 1H), 7.07-7.03 (m, 1H), |
| | (trifluoromethyl)tetrahydrofuran- | | 6.90 (dd, J = 16.7, 9.4 Hz, |
| | 2-carboxamide | | 1H), 5.00 (d, J = 11.0 Hz, |
| | | | 1H), 4.06 (dd, J = 11.0, |
| | | | 8.2 Hz, 1H), 3.99 (d, J = |
| | | | 2.7 Hz, 3H), 3.86-3.83 |
| | | | (m, 2H), 3.40 (dd, J = 6.0, |
| | | | 4.1 Hz, 2H), 2.77-2.69 |
| | | | (m, 1H), 2.54 (t, J = 5.2 |
| | | | Hz, 2H), 2.44-2.37 (m, |
| | | | 2H), 2.33 (s, 3H), 1.67 (s, |
| | | | 3H), 0.79-0.76 (m, 3H) |
| | | | ppm. |
| 12 | (2R,3S,4S,5R)-3-(3,4-difluoro- | ESI-MS m/z calc. | ¹H NMR (400 MHz, |
| | 2-methoxyphenyl)-4,5- | 570.551, found 571.26 | Chloroform-d) δ 8.59 (s, |
| | dimethyl-N-(5-methyl-2-(4- | (M + 1)⁺; Retention time: | 1H), 8.38 (s, 1H), 8.35 (s, |
| | methylpiperazine-1- | 2.5 minutes. | 1H), 7.10-7.06 (m, 1H), |
| | carbonyl)pyridin-4-yl)-5- | | 6.92-6.86 (m, 1H), 5.04 |
| | (trifluoromethyl)tetrahydrofuran- | | (d, J = 11.0 Hz, 1H), 4.06 |
| | 2-carboxamide | | (dd, J = 11.0, 7.8 Hz, |
| | | | 1H), 4.00 (d, J = 2.7 Hz, |
| | | | 3H), 3.81-3.72 (m, 2H), |
| | | | 3.50 (t, J = 4.8 Hz, 2H), |
| | | | 2.79-2.72 (m, 1H), 2.51- |
| | | | 2.43 (m, 2H), 2.40-2.31 |
| | | | (m, 2H), 2.30 (s, 3H), |
| | | | 2.29 (s, 3H), 1.67 (s, 3H), |
| | | | 0.80-0.77 (m, 3H) ppm. |
| 13 | (2R,3S,4S,5R)-3-(3,4-difluoro- | ESI-MS m/z calc. | ¹H NMR (400 MHz, |
| | 2-methoxyphenyl)-4,5- | 557.513, found 558.22 | Chloroform-d) δ 9.07 (s, |
| | dimethyl-N-(4-(4- | (M + 1)⁺; Retention time: | 1H), 8.75 (d, J = 5.0 Hz, |
| | methylpiperazine-1- | 2.30 minutes. | 1H), 7.29 (d, J = 5.0 Hz, |
| | carbonyl)pyrimidin-2-yl)-5- | | 1H), 7.11-7.07 (m, 1H), |
| | (trifluoromethyl)tetrahydrofuran- | | 6.89-6.83 (m, 1H), 5.02 |
| | 2-carboxamide | | (d, J = 10.5 Hz, 1H), 4.11 |
| | | | (dd, J = 10.8, 8.0 Hz, |
| | | | 1H), 3.99 (d, J = 2.7 Hz, |
| | | | 3H), 3.85-3.78 (m, 2H), |
| | | | 3.70-3.59 (m, 2H), 2.79- |
| | | | 2.71 (m, 1H), 2.57-2.52 |
| | | | (m, 2H), 2.47-2.42 (m, |
| | | | 2H), 2.34 (s, 3H), 1.68 (s, |
| | | | 3H), 0.78 (dd, J = 7.6, 2.1 |
| | | | Hz, 3H) ppm. |

The following compound was made using the method described in Example 2, except that the conditions used in the amide coupling step 8 were those described in Example 6 step 4, using (2-aminopyridin-4-yl) (4-methylpiperazin-1-yl) methanone as the coupling partner in chloroform as the solvent. Steps 9 to 12 were omitted:

| Cmpd No. | Compound Name | LC/MS | NMR (shifts in ppm) |
|---|---|---|---|
| 14 | (2R,3S,4S,5R)-3-(3,4-difluoro- | ESI-MS m/z calc. | ¹H NMR (400 MHz, |
| | 2-methoxyphenyl)-4,5- | 556.525, found 557.28 | Chloroform-d) δ 8.99 (s, |
| | dimethyl-N-(4-(4- | (M + 1)⁺; Retention time: | 1H), 8.37 (d, J = 5.0 Hz, |
| | methylpiperazine-1- | 2.5 minutes. | 1H), 8.16 (s, 1H), 7.11- |
| | carbonyl)pyridin-2-yl)-5- | | 7.07 (m, 1H), 7.04 (dd, J = |
| | (trifluoromethyl)tetrahydrofuran- | | 5.0, 1.4 Hz, 1H), 6.90 |
| | 2-carboxamide | | (dd, J = 16.9, 9.2 Hz, |
| | | | 1H), 5.00 (d, J = 11.0 Hz, |
| | | | 1H), 4.09 (dd, J = 11.0, |
| | | | 8.2 Hz, 1H), 3.99 (d, J = |
| | | | 2.7 Hz, 3H), 3.83-3.70 |
| | | | (m, 2H), 3.41-3.34 (m, |
| | | | 2H), 2.77-2.69 (m, 1H), |
| | | | 2.55-2.45 (m, 2H), 2.39- |
| | | | 2.31 (m, 5H), 1.68 (s, |
| | | | 3H), 0.79-0.77 (m, 3H) |
| | | | ppm. |

The following compounds were made using the method described in Example 2, except that different amines were used in the amide coupling step 8. Steps 9 to 12 were omitted. A final Boc deprotection step was carried out at ambient temperature over 2 h using an excess of TFA in DCM, conditions well known in the art:

| Cmpd No. | Compound Name | LC/MS | NMR (shifts in ppm) |
|---|---|---|---|
| 15 | (2R,3S,4S,5R)-3-(3,4-difluoro- | ESI-MS m/z calc. | ¹H NMR (400 MHz, |
| | 2-methoxy-phenyl)-4,5- | 556.525, found 557.27 | Chloroform-d) δ 8.60 (br |
| | dimethyl-N-[5-methyl-2- | (M + 1)⁺; Retention time: | s, 1H), 8.39 (s, 1H), 8.35 |
| | (piperazine-1-carbonyl)-4- | 2.31 minutes. | (s, 1H), 7.10-7.06 (m, |
| | pyridyl]-5- | | 1H), 6.89 (dd, J = 16.9, |
| | (trifluoromethyl)tetrahydrofuran- | | 9.2 Hz, 1H), 5.04 (d, J = |
| | 2-carboxamide | | 11.4 Hz, 1H), 4.06 (dd, J = |
| | | | 10.9, 7.7 Hz, 1H), 4.00 |
| | | | (d, J = 2.7 Hz, 3H), 3.80- |
| | | | 3.72 (m, 2H), 3.52-3.49 |
| | | | (m, 2H), 2.96 (t, J = 4.8 |
| | | | Hz, 2H), 2.87-2.83 (m, |
| | | | 2H), 2.79-2.72 (m, 1H), |
| | | | 2.29 (s, 3H), 1.67 (s, 3H), |
| | | | 0.80-0.78 (m, 3H) ppm; |
| | | | amine NH not observed. |
| 16 | (2R,3S,4S,5R)-3-(3,4-difluoro- | ESI-MS m/z calc. | ¹H NMR (400 MHz, |
| | 2-methoxy-phenyl)-N-[5- | 560.489, found 561.25 | Chloroform-d) δ 8.86 (br |
| | fluoro-2-(piperazine-1- | (M + 1)⁺; Retention time: | s, 1H), 8.57 (d, J = 6.0 |
| | carbonyl)-4-pyridyl]-4,5- | 2.32 minutes | Hz, 1H), 8.40 (d, J = 1.8 |
| | dimethyl-5- | | Hz, 1H), 7.08-7.05 (m, |
| | (trifluoromethyl)tetrahydrofuran- | | 1H), 6.89 (dd, J = 16.7, |
| | 2-carboxamide | | 8.9 Hz, 1H), 5.03 (d, J = |
| | | | 11.0 Hz, 1H), 4.06 (dd, J = |
| | | | 11.0, 8.2 Hz, 1H), 4.00 |
| | | | (d, J = 2.3 Hz, 3H), 3.74 |
| | | | (t, J = 4.6 Hz, 2H), 3.48- |
| | | | 3.45 (m, 2H), 2.95 (t, J = |
| | | | 4.8 Hz, 2H), 2.86-2.82 |
| | | | (m, 2H), 2.79-2.71 (m, |
| | | | 1H), 1.67 (s, 3H), 0.78 |
| | | | (dd, J = 7.3, 1.8 Hz, 3H) |
| | | | ppm; amine NH not |
| | | | observed. |
| 17 | (2R,3S,4S,5R)-3-(3,4-difluoro- | ESI-MS m/z calc. | ¹H NMR (400 MHz, |
| | 2-methoxyphenyl)-4,5- | 542.498, found 543.26 | Chloroform-d) δ 8.87 (d, |
| | dimethyl-N-(6-(piperazine-1- | (M + 1)⁺; Retention time: | J = 16.0 Hz, 1H), 8.18 (t, |
| | carbonyl)pyridin-2-yl)-5- | 2.35 minutes | J = 8.5 Hz, 1H), 7.74 (t, |
| | (trifluoromethyl)tetrahydrofuran- | | J = 8.0 Hz, 1H), 7.27 (s, |
| | 2-carboxamide | | 1H), 7.11-7.07 (m, 1H), |
| | | | 6.93-6.86 (m, 1H), 5.00 |
| | | | (d, J = 11.0 Hz, 1H), 4.09 |
| | | | (dd, J = 10.8, 8.0 Hz, |
| | | | 1H), 3.98 (d, J = 2.7 Hz, |
| | | | 3H), 3.85-3.75 (m, 2H), |
| | | | 3.46 (dd, J = 5.7, 3.9 Hz, |
| | | | 2H), 2.99 (t, J = 4.8 Hz, |
| | | | 2H), 2.84 (dd, J = 15.8, |
| | | | 4.8 Hz, 2H), 2.77-2.69 |
| | | | (m, 1H), 1.67 (s, 3H), |
| | | | 0.79-0.77 (m, 3H) ppm; |
| | | | amine NH not observed. |

The following compounds were made using the method described in Example 2, except that the conditions used in the amide coupling step 8 were similar to those described in Example 6 step 4, carrying out the reaction at 80° C. for 48 h, using different amines as the coupling partner and acetonitrile as the solvent. Steps 9 to 12 were omitted. A final Boc deprotection step was carried out at ambient temperature using an excess of TFA in DCM, conditions well known in the art:

| Cmpd No. | Compound Name | LC/MS | NMR (shifts in ppm) |
|---|---|---|---|
| 18 | (2R,3S,4S,5R)-3-(3,4-difluoro- | ESI-MS m/z calc. | ¹H NMR (400 MHz, |
| | 2-methoxyphenyl)-4,5- | 542.498, found 543.26 | Chloroform-d) δ 8.98 (br |
| | dimethyl-N-(4-(piperazine-1- | (M + 1)⁺; Retention time: | s, 1H), 8.37 (dd, J = 5.0, |
| | carbonyl)pyridin-2-yl)-5- | 2.32 minutes. | 0.9 Hz, 1H), 8.16 (t, J = |
| | (trifluoromethyl)tetrahydrofuran- | | 1.1 Hz, 1H), 7.10-7.04 |
| | 2-carboxamide | | (m, 2H), 6.93-6.86 (m, |
| | | | 1H), 5.00 (d, J = 11.0 Hz, |
| | | | 1H), 4.08 (dd, J = 11.0, |
| | | | 8.2 Hz, 1H), 3.99 (d, J = |
| | | | 2.7 Hz, 3H), 3.80-3.68 |
| | | | (m, 2H), 3.40-3.28 (m, |
| | | | 2H), 2.99-2.91 (m, 2H), |
| | | | 2.84-2.78 (m, 2H), 2.77- |
| | | | 2.69 (m, 1H), 1.68 (s, |
| | | | 3H), 0.79-0.76 (m, 3H) |
| | | | ppm; amine NH not |
| | | | observed. |
| 19 | (2R,3S,4S,5R)-3-(3,4-difluoro- | ESI-MS m/z calc. | ¹H NMR (400 MHz, |
| | 2-methoxyphenyl)-4,5- | 543.486, found 544.24 | Chloroform-d) δ 9.07 (br |
| | dimethyl-N-(2-(piperazine-1- | (M + 1)⁺; Retention time: | s, 1H), 8.62 (d, J = 6.0 |
| | carbonyl)pyrimidin-4-yl)-5- | 2.28 minutes. | Hz, 1H), 8.11 (d, J = 6.0 |
| | (trifluoromethyl)tetrahydrofuran- | | Hz, 1H), 7.08-7.04 (m, |
| | 2-carboxamide | | 1H), 6.93-6.87 (m, 1H), |
| | | | 5.01 (d, J = 11.0 Hz, 1H), |
| | | | 4.06 (dd, J = 11.0, 8.2 Hz, |
| | | | 1H), 3.99 (d, J = 2.7 Hz, |
| | | | 3H), 3.81 (t, J = 5.0 Hz, |
| | | | 2H), 3.36 (q, J = 3.2 Hz, |
| | | | 2H), 3.00 (t, J = 5.0 Hz, |
| | | | 2H), 2.89 (t, J = 5.0 Hz, |
| | | | 2H), 2.77-2.69 (m, 1H), |
| | | | 1.67 (s, 3H), 0.79-0.77 |
| | | | (m, 3H) ppm; amine NH |
| | | | not observed. |
| 20 | (2R,3S,4S,5R)-3-(3,4-difluoro- | ESI-MS m/z calc. | ¹H NMR (400 MHz, |
| | 2-methoxyphenyl)-4,5- | 543.486, found 544.22 | Chloroform-d) δ 9.08 (br |
| | dimethyl-N-(4-(piperazine-1- | (M + 1)⁺; Retention time: | s, 1H), 8.76 (d, J = 4.6 |
| | carbonyl)pyrimidin-2-yl)-5- | 2.17 minutes. | Hz, 1H), 7.29 (d, J = 5.0 |
| | (trifluoromethyl)tetrahydrofuran- | | Hz, 1H), 7.11-7.07 (m, |
| | 2-carboxamide | | 1H), 6.89-6.83 (m, 1H), |
| | | | 5.02 (d, J = 11.0 Hz, 1H), |
| | | | 4.11 (dd, J = 10.5, 7.8 Hz, |
| | | | 1H), 3.99 (d, J = 2.7 Hz, |
| | | | 3H), 3.78 (dd, J = 6.0, 3.7 |
| | | | Hz, 2H), 3.58 (dd, J = |
| | | | 5.5, 4.1 Hz, 2H), 2.99 (t, J = |
| | | | 5.0 Hz, 2H), 2.90 (t, J = |
| | | | 4.8 Hz, 2H), 2.79-2.71 |
| | | | (m, 1H), 0.79-0.77 (m, |
| | | | 3H) ppm; 1 Me signal |
| | | | coincides with water |
| | | | signal; amine NH not |
| | | | observed. |
| 21 | (2R,3S,4S,5R)-3-(3,4-difluoro- | ESI-MS m/z calc. | ¹H NMR (400 MHz, |
| | 2-methoxyphenyl)-4,5- | 543.486, found 544.23 | Chloroform-d) δ 9.04 (br |
| | dimethyl-N-(6-(piperazine-1- | (M + 1)⁺; Retention time: | s, 1H), 8.89 (d, J = 0.9 |
| | carbonyl)pyrimidin-4-yl)-5- | 2.27 minutes | Hz, 1H), 8.27 (d, J = 0.7 |
| | (trifluoromethyl)tetrahydrofuran- | | Hz, 1H), 7.07-7.03 (m, |
| | 2-carboxamide | | 1H), 6.89 (dd, J = 16.5, |
| | | | 9.2 Hz, 1H), 5.01 (d, J = |
| | | | 11.0 Hz, 1H), 4.07 (dd, J = |
| | | | 11.0, 8.2 Hz, 1H), 4.00 |
| | | | (d, J = 2.7 Hz, 3H), 3.74 |
| | | | (dd, J = 5.5, 4.1 Hz, 2H), |
| | | | 3.41 (q, J = 4.0 Hz, 2H), |
| | | | 2.95 (t, J = 5.0 Hz, 2H), |
| | | | 2.85 (t, J = 12.8 Hz, 2H), |
| | | | 2.78-2.70 (m, 1H), 1.68 |
| | | | (s, 3H), 0.79-0.77 (m, |
| | | | 3H) ppm; amine NH not |
| | | | observed. |

The following compound was made using a methods similar to those described in Example 2, except that the conditions used in the amide coupling step 8 were similar to those described in Example 6 step 4, carrying out the reaction at 80° C. for 48 h, using tert-butyl 4-(6-aminopyrimidine-4-carbonyl) piperazine-1-carboxylate as the coupling partner and acetonitrile as the solvent. Steps 9 to 12 were omitted. The product of step 8 was Boc-deprotected at ambient temperature over 2 h using an excess of TFA in DCM and N-methylated via reductive amination using formaldehyde, sodium triacetoxyborohydride and acetic acid in methanol at ambient temperature over 90 min, conditions well known in the art:

| Cmpd No. | Compound Name | LC/MS | NMR (shifts in ppm) |
|---|---|---|---|
| 22 | (2R,3S,4S,5R)-3-(3,4-difluoro- | ESI-MS m/z calc. | ¹H NMR (400 MHz, |
| | 2-methoxyphenyl)-4,5- | 557.513, found 556.12 | Chloroform-d) δ 9.04 (br |
| | dimethyl-N-(6-(4- | (M − 1)⁻; Retention time: | s, 1H), 8.88 (d, J = 1.4 |
| | methylpiperazine-1- | 2.42 minutes. | Hz, 1H), 8.28 (d, J = 1.4 |
| | carbonyl)pyrimidin-4-yl)-5- | | Hz, 1H), 7.07-7.03 (m, |
| | (trifluoromethyl)tetrahydrofuran- | | 1H), 6.92-6.86 (m, 1H), |
| | 2-carboxamide | | 5.01 (d, J = 11.0 Hz, 1H), |
| | | | 4.07 (dd, J = 11.0, 8.2 Hz, |
| | | | 1H), 4.00 (d, J = 3.2 Hz, |
| | | | 3H), 3.84-3.76 (m, 2H), |
| | | | 3.52-3.46 (m, 2H), 2.78- |
| | | | 2.70 (m, 1H), 2.59-2.52 |
| | | | (m, 2H), 2.48-2.40 (m, |
| | | | 2H), 2.35 (s, 3H), 1.68 (s, |
| | | | 3H), 0.79-0.77 (m, 3H) |
| | | | ppm. |

The following compound was made using methods similar to those described in Example 2, except that for step 8 ammonium hydroxide was used as the amide coupling partner in place of methyl 6-aminopyridine-2-carboxylate. The product of step 8 was reacted with methyl 4-chloro-5-trimethylsilyl-pyridine-2-carboxylate using palladium catalysed amination conditions that are well known in the art (40 mol % Pd(OAc)₂, 80 mol % Xantphos, cesium carbonate, dioxane, 100° C., 11 h) followed by ester amination using the conditions described in step 9. Steps 10 to 12 were omitted:

| Cmpd No. | Compound Name | LC/MS | NMR (shifts in ppm) |
|---|---|---|---|
| 23 | 4-((2R,3S,4S,5R)-3-(3,4- | ESI-MS m/z calc. | ¹H NMR (400 MHz, |
| | difluoro-2-methoxyphenyl)-4,5- | 545.574, found 546.21 | Chloroform-d) δ 8.94 (s, |
| | dimethyl-5- | (M + 1)⁺; Retention time: | 1H), 8.71 (s, 1H), 8.49 (s, |
| | (trifluoromethyl)tetrahydrofuran- | 3.15 minutes. | 1H), 7.96 (br s, 1H), |
| | 2-carboxamido)-5- | | 7.08-7.04 (m, 1H), 6.93- |
| | (trimethylsilyl)picolinamide | | 6.86 (m, 1H), 5.53 (br s, |
| | | | 1H), 5.05 (d, J = 11.2 Hz, |
| | | | 1H), 4.07 (dd, J = 11.2, |
| | | | 8.2 Hz, 1H), 4.01 (d, J = |
| | | | 2.7 Hz, 3H), 2.80-2.72 |
| | | | (m, 1H), 1.65 (s, 3H), |
| | | | 0.79-0.77 (m, 3H), 0.48 |
| | | | (s, 9H) ppm. |

The following compound can be made using a method similar to that described in Example 2, except that rac-(1S,2R)-6-fluoro-1,2-dimethyl-2-(trifluoromethyl)-1,2-dihydro-4H-furo[2,3-c]chromen-4-one would be used as the starting material in place of rac-(1S,2R)-6,7-difluoro-1,2-dimethyl-2-(trifluoromethyl)-1,2-dihydro-4H-furo[2,3-c]chromen-4-one. Steps 10 to 12 would be omitted. Purification would be performed by recrystallization to give Compound 303:

| | | | |
|---|---|---|---|
| Cmpd No. | Compound Name | LC/MS | NMR (shifts in ppm) |

| | | |
|---|---|---|
| 303 | 4-((2R,3S,4S,5R)-3-(3-fluoro- | ¹H NMR (400 MHz, |
| | 2-methoxyphenyl)-4,5-imethyl- | DMSO-d6) δ 10.79 (s, |
| | 5-(trifluoromethyl) | 1H), 8.50 (d, J = 5.5 Hz, |
| | tetrahydrofuran-2- | 1H), 8.28 (d, J = 2.2 Hz, |
| | carboxamido)picolinamide | 1H), 8.07 (d, J = 2.7 Hz, |
| | | 1H), 7.83 (dd, J = 5.5, 2.2 |
| | | Hz, 1H), 7.62 (d, J = 3.0 |
| | | Hz, 1H), 7.21 (ddd, J = |
| | | 11.7, 7.9, 1.8 Hz, 1H), |
| | | 7.14 (d, J = 1.8 Hz, 1H), |
| | | 7.11 (dt, J = 7.9, 4.0 Hz, |
| | | 1H), 5.11 (d, J = 10.3 Hz, |
| | | 1H), 4.33 (dd, J = 10.4, |
| | | 7.6 Hz, 1H), 3.88 (d, J = |
| | | 1.9 Hz, 3H), 2.80 (t, J = |
| | | 7.5 Hz, 1H), 1.62 (s, 3H), |
| | | 0.79-0.53 (m, 4H) ppm. |

The following compound can be made using a method similar to that described in Example 2, except that rac-(1S,2R)-7-fluoro-1,2-dimethyl-2-(trifluoromethyl)-1,2-dihydro-4H-furo[2,3-c]chromen-4-one would be used as the starting material in place of rac-(1S,2R)-6,7-difluoro-1,2-dimethyl-2-(trifluoromethyl)-1,2-dihydro-4H-furo[2,3-c]chromen-4-one. Steps 10 to 12 would be omitted. Purification would be performed by recrystallization to give Compound 304:

| | | | |
|---|---|---|---|
| Cmpd No. | Compound Name | LC/MS | NMR (shifts in ppm) |

| | | |
|---|---|---|
| 304 | 4-((2R,3S,4S,5R)-3-(4-fluoro- | ¹H NMR (400 MHz, |
| | 2-methoxyphenyl)-4,5- | DMSO-d6) δ 10.78 (s, |
| | dimethyl-5-(trifluoromethyl) | 1H), 8.50 (d, J = 5.5 Hz, |
| | tetrahydrofuran-2- | 1H), 8.27 (d, J = 2.2 Hz, |
| | carboxamido)picolinamide | 1H), 8.15-8.00 (m, 1H), |
| | | 7.91-7.74 (m, 1H), 7.62 |
| | | (d, J = 2.7 Hz, 1H), 7.40- |
| | | 7.14 (m, 1H), 6.93 (dd, J = |
| | | 11.3, 2.7 Hz, 1H), 6.77 |
| | | (td, J = 8.5, 2.6 Hz, 1H), |
| | | 5.12 (d, J = 10.3 Hz, 1H), |
| | | 4.21 (dd, J = 10.3, 7.5 Hz, |
| | | 1H), 3.83 (s, 3H), 2.82 (t, |
| | | J = 7.5 Hz, 1H), 1.60 (s, |
| | | 3H), 0.67 (t, J = 4.3 Hz, |
| | | 4H) ppm. |

### Example 3

### rel-4-((6S,7R)-7-(4-fluoro-2-methoxy-3-methylphenyl)-2,5-dioxaspiro[3.4]octane-6-carboxamido) picolinamide (24) and rel-4-((6R,7S)-7-(4-fluoro-2-methoxy-3-methylphenyl)-2,5-dioxaspiro[3.4]octane-6-carboxamido) picolinamide (25)

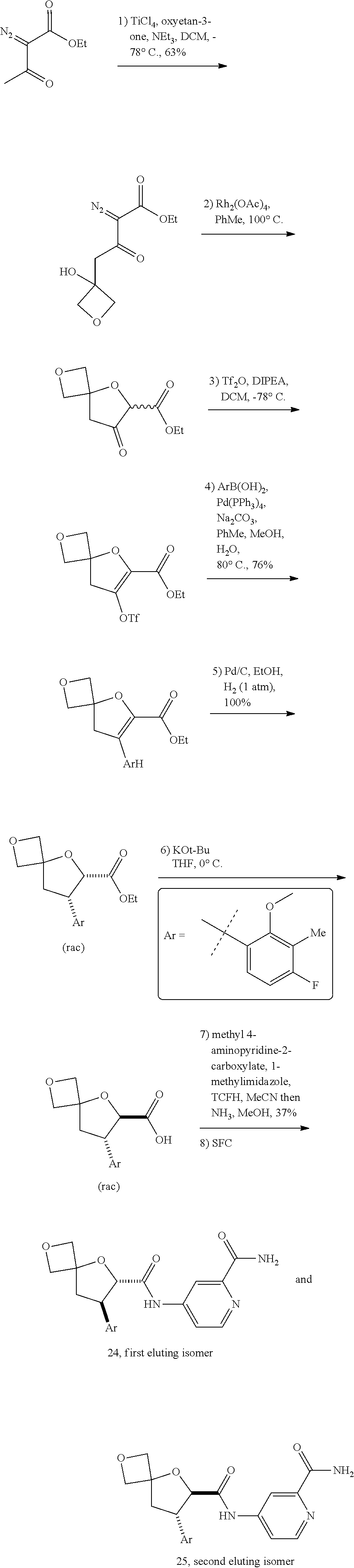

### Step 1:

TiCl₄ (1.2 mL of 1 M, 1.20 mmol) and Et₃N (170 μL, 1.22 mmol) were added dropwise to a stirred solution of ethyl 2-diazo-3-oxobutanoate (150 μL, 1.09 mmol) in DCM (6 mL) at −78° C. The reaction mixture was stirred for 1 h before a solution of Ti(OiPr)₄ (325 μL, 1.10 mmol) and oxetan-3-one (70 μL, 1.09 mmol) in DCM (1.5 mL) was added via cannula. The reaction was stirred for an additional 5 h. The mixture was quenched by addition of a saturated aqueous NH₄Cl solution. The aqueous layer was separated and extracted with DCM. The combined organic phases were washed with brine, dried over MgSO₄, filtered, and concentrated in vacuo. Purification by flash chromatography (24 g SiO₂, 0 to 70% EtOAc in heptane) gave ethyl 2-diazo-4-(1-hydroxycyclobutyl)-3-oxobutanoate (156 mg, 63%). ¹H NMR (400 MHZ, Chloroform-d) δ 4.73-4.65 (m, 2H), 4.47 (d, J=7.4 Hz, 2H), 4.33 (q, J=7.1 Hz, 2H), 3.80 (s, 1H), 3.49 (s, 2H), 1.35 (t, J=7.1 Hz, 3H) ppm.

### Step 2:

A suspension of Rh₂(OAc)₄ (9.5 mg, 0.021 mmol) in toluene (4 mL) was heated at 100° C. for 10 min. A solution of ethyl 2-diazo-4-(1-hydroxycyclobutyl)-3-oxobutanoate (250 mg, 1.10 mmol) in toluene (3 mL) was added dropwise and the reaction was stirred for 45 min. The mixture was cooled to ambient temperature and concentrated in vacuo to give ethyl rac-7-oxo-2,5-dioxaspiro[3.4]octane-6-carboxylate (219 mg) which was used in the next step without further purification. ¹H NMR (500 MHZ, Chloroform-d) δ 5.14 (dt, J=7.3, 0.7 Hz, 1H), 4.99-4.92 (m, 1H), 4.68 (dt, J=7.2, 1.1 Hz, 1H), 4.61 (dt, J=6.6, 0.9 Hz, 1H), 4.60 (s, 1H), 4.24 (qd, J=7.1, 4.4 Hz, 2H), 3.05 (d, J=19.2 Hz, 1H), 2.90 (d, J=18.9 Hz, 1H), 1.30 (t, J=7.1 Hz, 3H) ppm.

### Step 3:

Trifluoromethylsulfonyl trifluoromethanesulfonate (1.7 mL of 1 M, 1.70 mmol) was added dropwise to a stirred solution of ethyl 7-oxo-2,5-dioxaspiro[3.4]octane-6-carboxylate (263 mg, 1.31 mmol) and DIPEA (700 μL, 4.019 mmol) in DCM (12 mL) at −78° C. The reaction mixture was stirred for 4 h at −78° C. The reaction was quenched by addition of a saturated aqueous NH₄Cl solution. The aqueous layer was separated and extracted with DCM. The combined organic layers were dried over MgSO₄, filtered, and concentrated in vacuo to give ethyl 7-(((trifluoromethyl) sulfonyl)oxy)-2,5-dioxaspiro[3.4]oct-6-ene-6-carboxylate, which was used as such, without further purification. ¹H NMR (400 MHZ, Chloroform-d) δ 5.03-4.95 (m, 2H), 4.67-4.59 (m, 2H), 4.36 (q, J=7.2 Hz, 2H), 3.43 (s, 2H), 1.36 (t, J=7.1 Hz, 3H) ppm.

### Step 4:

Ethyl 7-(((trifluoromethyl) sulfonyl)oxy)-2,5-dioxaspiro[3.4]oct-6-ene-6-carboxylate (436.6 mg, 1.31 mmol), (4-fluoro-2-methoxy-3-methylphenyl) boronic acid (Intermediate G, 290 mg, 1.58 mmol), Pd(PPh₃)₄ (90 mg, 0.078 mmol) and Na₂CO₃ (350 mg, 3.30 mmol) were dissolved in a mixture of toluene (7.5 mL), MeOH (750 μL) and H₂O (750 μL). The mixture was degassed and heated at 80° C. for 16 h. The reaction was cooled to ambient temperature, diluted with EtOAc, and washed with brine. The organic layer was dried over MgSO₄, filtered and concentrated in vacuo. Purification by flash chromatography (40 g SiO₂, 0 to 30% EtOAc in heptane) gave ethyl 7-(4-fluoro-2-methoxy-3-methylphenyl)-2,5-dioxaspiro[3.4]oct-6-ene-6-carboxylate (356 mg, 76%) as a pale yellow oil. ¹H NMR (500 MHz, Chloroform-d) δ 6.98 (dd, J=8.6, 6.4 Hz, 1H), 6.80 (t, J=8.7 Hz, 1H), 5.11-5.06 (m, 2H), 4.70-4.63 (m, 2H), 4.17 (q, J=7.1 Hz, 2H), 3.63 (s, 3H), 3.45 (s, 2H), 2.20 (d, J=2.1 Hz, 3H), 1.14 (t, J=7.1 Hz, 3H) ppm. ESI-MS m/z calc. 322.12164, found 323.6 (M+1)⁺; Retention time: 0.8 minutes.

### Step 5:

EtOH (12 mL) was added to a mixture of ethyl 7-(4-fluoro-2-methoxy-3-methylphenyl)-2,5-dioxaspiro[3.4]oct-6-ene-6-carboxylate (356 mg, 1.10 mmol) and Pd/C (110 mg, 0.10 mmol). The reaction mixture was degassed and stirred under a balloon of hydrogen for 3 days. The reaction mixture was filtered through a pad of Celite and washed with MeOH. The mother liquors were concentrated in vacuo to give ethyl rac-(6S,7S)-7-(4-fluoro-2-methoxy-3-methylphenyl)-2,5-dioxaspiro[3.4]octane-6-carboxylate (367 mg, 100%) as a white solid. ESI-MS m/z calc. 324.1373, found 325.4 (M+1)⁺; Retention time: 1.85 minutes.

### Step 6:

Potassium tert-butoxide (1.6 mL of 1 M solution in THF, 1.60 mmol) was added dropwise to a stirred solution of ethyl rac-(6S,7S)-7-(4-fluoro-2-methoxy-3-methylphenyl)-2,5-dioxaspiro[3.4]octane-6-carboxylate (185 mg, 0.57 mmol) in THF (6.4 mL) at 0° C. After 1 h, the reaction mixture was diluted with EtOAc. The mixture was then quenched by addition of 1 M aqueous HCl. The aqueous layer was separated and extracted with EtOAc. The combined organic phases were dried over MgSO₄, filtered and concentrated in vacuo to give rac-(6R,7S)-7-(4-fluoro-2-methoxy-3-methylphenyl)-2,5-dioxaspiro[3.4]octane-6-carboxylic acid (221 mg), which was used without further purification in the next step. ESI-MS m/z calc. 296.106, found 297.4 (M+1)⁺; Retention time: 0.4 minutes.

### Step 7:

Methyl 4-aminopyridine-2-carboxylate (33 mg, 0.22 mmol) was added to a stirred solution of rac-(6R,7S)-7-(4-fluoro-2-methoxy-3-methylphenyl)-2,5-dioxaspiro[3.4]octane-6-carboxylic acid (58.5 mg, 0.20 mmol) in MeCN (2 mL). 1-Methylimidazole (55 μL, 0.69 mmol) and TCFH (65 mg, 0.2317 mmol) were successively added to the reaction mixture. The solution was stirred at ambient temperature for 16 h. A methanolic ammonia solution (6 mL of 7 M, 42.00 mmol) was added and the reaction was stirred at ambient temperature for a further 24 h. The reaction mixture was diluted with EtOAc, washed with brine, dried over MgSO₄, filtered, and concentrated in vacuo onto silica gel. Purification by flash chromatography (24 g SiO₂, 0 to 100% EtOAc in heptane) gave rac-4-((6R,7S)-7-(4-fluoro-2-methoxy-3-methylphenyl)-2,5-dioxaspiro[3.4]octane-6-carboxamido) picolinamide (30 mg, 37%) as a colourless oil. ESI-MS m/z calc. 415.15436, found 416.7 (M+1)⁺; 414.7 (M−1); Retention time: 0.7 minutes.

### Step 8:

The enantiomers of rac-4-((6R,7S)-7-(4-fluoro-2-methoxy-3-methylphenyl)-2,5-dioxaspiro[3.4]octane-6-carboxamido) picolinamide were separated by chiral SFC using a Chiralpak AS-H column, 5 μm particle size, 25 cm×10 mm from Daicel Corporation (Mobile phase: 30% methanol (supplemented with 20 mM NH₃), 70% CO₂; System pressure: 100 bar) on a Minigram SFC instrument from Berger Instruments:

First Eluting Isomer (rt=3.09 min): rel-4-((6S,7R)-7-(4-fluoro-2-methoxy-3-methylphenyl)-2,5-dioxaspiro[3.4]octane-6-carboxamido) picolinamide (24, 4.5 mg, 29%). ¹H NMR (500 MHz, DMSO-d₆) δ 10.48 (s, 1H), 8.48 (d, J=5.4 Hz, 1H), 8.29 (d, 1H), 8.05 (d, J=2.8 Hz, 1H), 7.81 (dd, J=5.5, 2.2 Hz, 1H), 7.60 (d, J=2.8 Hz, 1H), 7.24 (dd, J=8.7, 6.6 Hz, 1H), 6.99 (t, J=8.8 Hz, 1H), 4.88 (d, J=6.8 Hz, 1H), 4.77 (d, J=6.9 Hz, 1H), 4.60 (d, J=6.7 Hz, 1H), 4.59-4.54 (m, 2H), 3.87 (q, J=7.6 Hz, 1H), 3.61 (s, 3H), 2.78 (dd, J=12.8, 7.6 Hz, 1H), 2.33 (dd, J=12.8, 8.7 Hz, 1H), 2.12 (d, J=2.0 Hz, 3H) ppm. ESI-MS m/z calc. 415.15436, found 416.3 (M+1)⁺; 414.3 (M−1); Retention time: 2.35 minutes.

Second Eluting Isomer (rt=4.08 min): rel-4-((6R,7S)-7-(4-fluoro-2-methoxy-3-methylphenyl)-2,5-dioxaspiro[3.4]octane-6-carboxamido) picolinamide, which required further purification by reverse phase preparative HPLC (25, 4.5 mg, 29%). ESI-MS m/z calc. 415.15436, found 416.3 (M+1)⁺; 414.2 (M−1); Retention time: 2.35 minutes.

### Example 4

### rel-4-((2R,3S,5R)-3-(3,4-difluoro-2-methoxyphenyl)-5-(methoxymethyl)-5-methyltetrahydrofuran-2-carboxamido) picolinamide (26), rel-4-((2S,3R,5S)-3-(3,4-difluoro-2-methoxyphenyl)-5-(methoxymethyl)-5-methyltetrahydrofuran-2-carboxamido) picolinamide (27), rel-4-((2R,3S,5S)-3-(3,4-difluoro-2-methoxyphenyl)-5-(methoxymethyl)-5-methyltetrahydrofuran-2-carboxamido) picolinamide (28), and rel-4-((2S,3R,5R)-3-(3,4-difluoro-2-methoxyphenyl)-5-(methoxymethyl)-5-methyltetrahydrofuran-2-carboxamido) picolinamide (29)

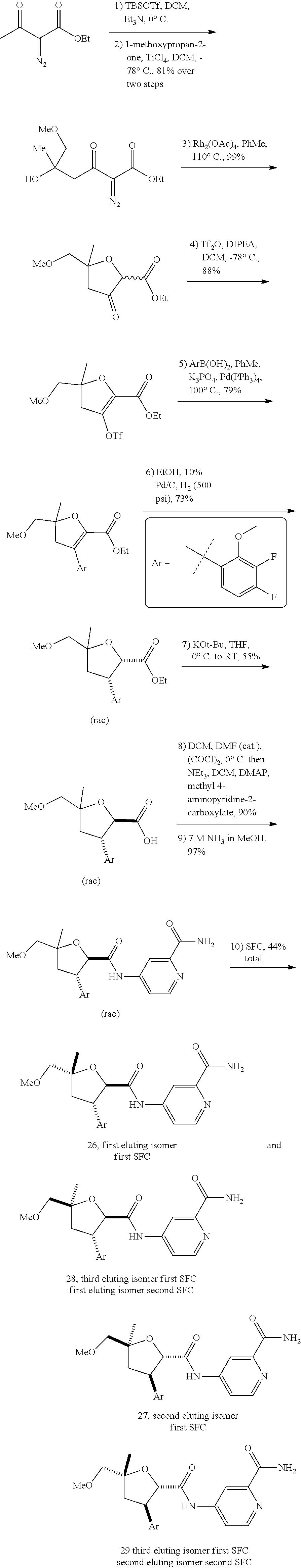

### Step 1:

Et₃N (9.56 g, 13.3 uA, 93.52 mmol) was added to a stirred solution of ethyl 2-diazo-3-oxobutanoate (6 g, 37.66 mmol) in DCM (50 mL) at 0° C. TBSOTf (11.95 g, 10.6 mL, 44.29 mmol) was added very slowly to the reaction mixture which was stirred further for 30 min at 0° C. The reaction mixture was washed with a 30% NaHCO₃ solution (200 mL). The organic layer was separated, washed with water (500 mL), dried over MgSO₄, filtered, and concentrated in vacuo to give ethyl 3-((tert-butyldimethylsilyl)oxy)-2-diazobut-3-enoate (10 g, 98%), which was used without purification.

### Step 2:

A solution of TiCl₄ (9.23 g, 5.4 mL, 48.16 mmol) in DCM (20 mL) was added dropwise to a stirred solution of 1-methoxypropan-2-one (4.36 g, 4.7 mL, 48.04 mmol) in DCM (40 mL) at −78° C. A solution of ethyl 3-((tert-butyldimethylsilyl)oxy)-2-diazobut-3-enoate (10 g, 36.982 mmol) in DCM (50 mL) was added dropwise to the reaction mixture at the same temperature. The reaction was stirred at −78° C. for 30 min before being quenched with water (250 mL). The organic layer was separated, dried over MgSO₄, filtered, and concentrated in vacuo. Purification by flash chromatography (SiO₂) gave ethyl rac-2-diazo-5-hydroxy-6-methoxy-5-methyl-3-oxohexanoate (7.5 g, 81%) as a light yellow oil. ¹H NMR (400 MHz, Chloroform-d) δ 4.28 (q, J=7.1 Hz, 2H), 3.79 (s, 1H), 3.37-3.26 (m, 6H), 2.84 (d, J=15.6 Hz, 1H), 1.31 (t, J=7.1 Hz, 3H), 1.23 (s, 3H) ppm. ESI-MS m/z calc. 244.1059, found 245.0 (M+1)⁺; Retention time: 2.71 minutes.

### Step 3:

A solution of ethyl rac-2-diazo-5-hydroxy-6-methoxy-5-methyl-3-oxohexanoate (7.5 g, 28.87 mmol) in toluene (230 mL) was added dropwise to a stirred and degassed solution of dirhodium tetraacetate (130 mg, 0.29 mmol) in toluene (70 mL) at 100° C. under N₂. The reaction mixture was heated to 110° C. for 10 min before being cooled to ambient temperature. The mixture was filtered through a pad of celite. The filtrate was collected and concentrated in vacuo to give ethyl rac-5-(methoxymethyl)-5-methyl-3-oxotetrahydrofuran-2-carboxylate (6.2 g, 99%) as a light brown oil and as a 1:1 mixture of diastereomers. ¹H NMR (400 MHZ, Chloroform-d) δ 4.70 (s, 1H), 4.28-4.15 (m, 2H), 3.50 (t, J=9.3 Hz, 1H), 3.31 (t, J=9.92 Hz, 1H), 3.30 (s, 3H) 2.67 (d, J=13.48 Hz, 1H), 2.35 (d, J=17.52 Hz, 1H), 1.39 (s, 3H), 1.27 (q, J=7.1 Hz, 3H) ppm.

### Step 4:

A solution of Tf₂O (8.97 g, 5.4 mL, 31.46 mmol) in DCM (15 mL) was added dropwise over 7 min to a stirred solution of ethyl rac-5-(methoxymethyl)-5-methyl-3-oxotetrahydrofuran-2-carboxylate (6.2 g, 28.67 mmol) and DIPEA (4.45 g, 6 mL, 34.45 mmol) in DCM (50 mL) at −78° C. The reaction mixture was stirred at −78° C. for 15 min, and then at 0° C. for a further 15 min. The reaction mixture was quenched by addition of a saturated NaHCO₃ solution (10 mL). The layers were separated and the aqueous phase was extracted with DCM (2×50 mL). The combined organic extracts were dried over MgSO₄, filtered, and concentrated in vacuo. Purification by flash chromatography gave ethyl rac-5-(methoxymethyl)-5-methyl-3-(((trifluoromethyl) sulfonyl)oxy)-4,5-dihydrofuran-2-carboxylate (9 g, 88%) as a light brown oil. ¹H NMR (400 MHZ, Chloroform-d) δ 4.33 (q, J=7.2 Hz, 2H), 3.46 (d, J=10.0 Hz, 1H), 3.39 (s, 3H), 3.35 (d, J=10.1 Hz, 1H), 3.20 (d, J=16.5 Hz, 1H), 2.70 (d, J=16.6 Hz, 1H), 1.44 (s, 3H), 1.33 (t, J=7.1 Hz, 3H) ppm. ESI-MS m/z calc. 348.0491, found 349.0 (M+1)⁺; Retention time: 3.59 minutes.

### Step 5:

Pd(PPh₃)₄ (614 mg, 0.53 mmol) was added to a stirred and argon degassed solution of ethyl rac-5-(methoxymethyl)-5-methyl-3-(((trifluoromethyl) sulfonyl)oxy)-4,5-dihydrofuran-2-carboxylate (3.7 g, 10.623 mmol) and (3,4-difluoro-2-methoxyphenyl) boronic acid (2.5 g, 13.302 mmol) in toluene (50 mL). The reaction mixture was further degassed and a 2 M aqueous K₃PO₄ solution (16 mL, 32.0 mmol) was added. The reaction mixture was heated for 3 h before being cooled down to ambient temperature. The mixture was filtered through a pad of celite. The filtrate was concentrated in vacuo. The residue was diluted in EtOAc (50 mL), washed with water and brine, dried over Na₂SO₄, filtered, and concentrated in vacuo. Purification by flash chromatography (SiO₂, 5 to 10% EtOAc in hexanes) gave ethyl rac-3-(3,4-difluoro-2-methoxyphenyl)-5-(methoxymethyl)-5-methyl-4,5-dihydrofuran-2-carboxylate (3 g, 79%) as a pale yellow oil. ¹H NMR (400 MHZ, Chloroform-d) δ 6.88-6.79 (m, 2H), 4.13 (q, J=7.1 Hz, 2H), 3.88 (d, J=1.24 Hz, 3H), 3.52 (d, J=9.9 Hz, 1H), 3.43 (d, J=4.8 Hz, 3H), 3.42 (d, J=9.6 Hz, 1H), 3.16 (d, J=16.7 Hz, 1H), 2.71 (d, J=16.7 Hz, 1H), 1.46 (s, 3H), 1.12 (t, J=7.1 Hz, 3H) ppm. ESI-MS m/z calc. 342.1279, found 343.0 (M+1)⁺; Retention time: 3.59 minutes.

### Step 6:

Pd/C (1 g, 8.23 mmol) was added to a stirred and argon degassed solution of ethyl rac-3-(3,4-difluoro-2-methoxyphenyl)-5-(methoxymethyl)-5-methyl-4,5-dihydrofuran-2-carboxylate (2 g, 5.84 mmol) in ethanol (50 mL). The reaction mixture was further degassed under argon for 5 min before being shaken in a Parr reactor under 500 psi hydrogen at ambient temperature. After 16 h, the reaction mixture was filtered through a pad of celite and the filtrate was concentrated in vacuo. Purification by flash chromatography (SiO₂, 10 to 15% EtOAc in hexanes) gave ethyl rac-(2S,3S)-3-(3,4-difluoro-2-methoxyphenyl)-5-(methoxymethyl)-5-methyltetrahydrofuran-2-carboxylate (1.5 g, 73%) as a light yellow oil and as a mixture of 4 stereoisomers. ESI-MS m/z calc. 344.1435, found 345.0 (M+1)⁺; Retention time: 3.62 minutes.

### Step 7:

Potassium tert-butoxide (1.5 g, 13.37 mmol) was added portionwise to a stirred solution of a mixture of stereoisomers of ethyl rac-(2S,3S)-3-(3,4-difluoro-2-methoxyphenyl)-5-(methoxymethyl)-5-methyltetrahydrofuran-2-carboxylate (1.3 g, 3.78 mmol) in dry THF (60 mL) at 0° C. under argon. The reaction mixture was stirred at 0° C. for 30 min before being warmed to ambient temperature. The reaction mixture was re-cooled to 0° C., quenched with a 2N HCl solution (2 mL) and extracted with EtOAc. The organic phase was collected, dried over MgSO₄, filtered, and concentrated in vacuo. Purification by flash chromatography (SiO₂, 40 to 50% EtOAc in hexanes) gave rac-(2R,3S)-3-(3,4-difluoro-2-methoxy phenyl)-5-(methoxymethyl)-5-methyltetrahydrofuran-2-carboxylic acid (700 mg, 55%) as a colourless oil and as a mixture of four stereoisomers. ¹H NMR (400 MHZ, DMSO-d₆) δ 12.50 (br s, 1H), 7.29-7.21 (m, 1H), 7.17-7.10 (m, 1H), 4.39 (d, J=9.56 Hz, 1H), 3.86 (s, 3H), 3.80 (d, J=10.12 Hz, 1H), 3.35 (d, J=9.56 Hz, 1H), 3.32 (s, 3H), 3.24 (d, J=9.56 Hz, 1H), 2.35 (dd, J=12.4, 8.0 Hz, 1H), 1.79 (t, J=11.8 Hz, 1H), 1.27 (d, J=12.44 Hz, 3H) ppm. ESI-MS m/z calc. 316.1122, found 317.0 (M+1)⁺; Retention time: 1.45 minutes.

### Step 8:

Oxalyl chloride (450 μL of 2 M, 0.9000 mmol) was added to a stirred solution of a mixture of rac-(2R,3S)-3-(3,4-difluoro-2-methoxyphenyl)-5-(methoxymethyl)-5-methyltetrahydrofuran-2-carboxylic acid (90 mg, 0.27 mmol) and DMF (5 μL, 0.065 mmol) in DCM (2.6 mL) at 0° C. The reaction mixture was stirred for 20 min before being concentrated in vacuo. The residue was diluted with DCM (2.2 mL) and the resultant solution was added dropwise to a stirred solution of methyl 4-aminopyridine-2-carboxylate (65 mg, 0.43 mmol), DMAP (3 mg, 0.025 mmol) and Et₃N (300 μL, 2.15 mmol) in DCM (2 mL) at 0° C. After 10 min, the reaction was warmed to ambient temperature and stirred for 16 h. The reaction mixture was diluted with DCM, washed with a 1 M HCl solution, dried over MgSO₄, filtered, and concentrated in vacuo directly onto silica gel. Purification by flash chromatography (24 g SiO₂, 0 to 100% EtOAc in heptane) gave methyl rac-4-((2R,3S)-3-(3,4-difluoro-2-methoxyphenyl)-5-(methoxymethyl)-5-methyltetrahydrofuran-2-carboxamido) picolinate (120 mg, 90%) as a colourless oil in a 3:1 mixture of diastereoisomers. ESI-MS m/z calc. 450.16025, found 451.6 (M+1)⁺; 449.7 (M−1); Retention time: 0.88 minutes.

### Step 9:

A solution of methyl rac-4-((2R,3S)-3-(3,4-difluoro-2-methoxyphenyl)-5-(methoxymethyl)-5-methyltetrahydrofuran-2-carboxamido) picolinate (120 mg, 0.27 mmol) in methanolic ammonia (15 mL of 7 M, 105.0 mmol) was stirred at ambient temperature overnight. The reaction mixture was concentrated in vacuo to give rac-4-((2R,3S)-3-(3,4-difluoro-2-methoxyphenyl)-5-(methoxymethyl)-5-methyltetrahydrofuran-2-carboxamido) picolinamide (113 mg, 97%) in a 3:1 mixture of diastereoisomers. ESI-MS m/z calc. 435.16058, found 436.6 (M+1)⁺; 434.6 (M−1); Retention time: 0.82 minutes.

### Step 10:

The stereoisomers of rac-4-((2R,3S)-3-(3,4-difluoro-2-methoxyphenyl)-5-(methoxymethyl)-5-methyltetrahydrofuran-2-carboxamido) picolinamide were separated by chiral SFC using a Lux Cellulose-2 column, 5 μm particle size, 25 cm×21.2 mm from Phenomenex, Inc. (Mobile phase: 35% IPA:MeCN (in a 1:1 ratio, supplemented with 0.2% DMIPA), 65% CO₂; System pressure: 100 bar) on a Minigram SFC instrument from Berger Instruments to give:

First Eluting Isomer (rt=3.46 min): rel-4-((2R,3S,5R)-3-(3,4-difluoro-2-methoxyphenyl)-5-(methoxymethyl)-5-methyltetrahydrofuran-2-carboxamido) picolinamide (26, 5.7 mg, 5%). ESI-MS m/z calc. 435.16058, found 436.3 (M+1)⁺; 434.2 (M−1); Retention time: 2.68 minutes.

Second Eluting Isomer (rt=4.00 min): rel-4-((2S,3R,5S)-3-(3,4-difluoro-2-methoxyphenyl)-5-(methoxymethyl)-5-methyltetrahydrofuran-2-carboxamido) picolinamide, which required further purification by reverse phase preparative HPLC (27, 4.7 mg, 4%). ¹H NMR (500 MHZ, DMSO-d₆) δ 10.23 (s, 1H), 8.47 (d, J=5.5 Hz, 1H), 8.30 (d, J=2.2 Hz, 1H), 8.04 (d, J=2.9 Hz, 1H), 7.85 (dd, J=5.5, 2.2 Hz, 1H), 7.58 (d, J=2.9 Hz, 1H), 7.28-7.22 (m, 1H), 7.22-7.13 (m, 1H), 4.50 (d, J=9.6 Hz, 1H), 4.03-3.92 (m, 1H), 3.81 (d, J=1.7 Hz, 3H), 3.41 (s, 2H), 3.36 (s, 3H), 2.19 (t, J=11.9 Hz, 1H), 2.15-2.06 (m, 1H), 1.34 (s, 3H) ppm. ESI-MS m/z calc. 435.16058, found 436.3 (M+1)⁺; 434.2 (M−1); Retention time: 2.68 minutes.

Third Eluting Isomer (rt=4.53 min): a mixture of both rel-4-((2R,3S,5S)-3-(3,4-difluoro-2-methoxyphenyl)-5-(methoxymethyl)-5-methyltetrahydrofuran-2-carboxamido) picolinamide (28) and rel-4-((2S,3R,5R)-3-(3,4-difluoro-2-methoxyphenyl)-5-(methoxymethyl)-5-methyltetrahydrofuran-2-carboxamido) picolinamide (29), which required further separation.

The third eluting peak, containing the 2 enantiomers of the major diastereoisomer, was further separated by chiral SFC using a Chiralcel OD-H column, 5 μm particle size, 25 cm×10 mm from Daicel Corporation (Mobile phase: 5% MeCN: IPA (in a 1:1 ratio, supplemented with 0.2% DMIPA), 95% CO₂; System Pressure: 100 bar) on a Minigram SFC instrument from Berger Instruments to give:

First Eluting Isomer (rt=3.48 min): rel-4-((2R,3S,5S)-3-(3,4-difluoro-2-methoxyphenyl)-5-(methoxymethyl)-5-methyltetrahydrofuran-2-carboxamido) picolinamide (28, 20.5 mg, 18%). ESI-MS m/z calc. 435.16058, found 436.3 (M+1)⁺; 434.3 (M−1); Retention time: 2.77 minutes.

Second Eluting Isomer (rt=4.55 min): rel-4-((2S,3R,5R)-3-(3,4-difluoro-2-methoxyphenyl)-5-(methoxymethyl)-5-methyltetrahydrofuran-2-carboxamido) picolinamide (29, 20.0 mg, 17%). ¹H NMR (500 MHZ, DMSO-d₆) δ 10.10 (s, 1H), 8.48 (d, J=5.5 Hz, 1H), 8.30 (d, J=2.2 Hz, 1H), 8.05 (d, J=2.9 Hz, 1H), 7.81 (dd, J=5.5, 2.2 Hz, 1H), 7.60 (d, J=2.9 Hz, 1H), 7.34-7.25 (m, 1H), 7.22-7.09 (m, 1H), 4.56 (d, J=9.0 Hz, 1H), 4.06-3.96 (m, 1H), 3.83 (d, J=1.6 Hz, 3H), 3.51 (d, J=9.6 Hz, 1H), 3.39 (d, 1H), 3.34 (s, 3H), 2.42 (dd, J=12.4, 8.2 Hz, 1H), 1.92 (dd, J=12.5, 11.2 Hz, 1H), 1.36 (s, 3H) ppm. ESI-MS m/z calc. 435.16058, found 436.3 (M+1)⁺; 434.3 (M−1); Retention time: 2.77 minutes.

The following compounds were made using the method described in Example 4, except that in step 2, 4-methoxybutan-2-one was used in place of 1-methoxypropan-2-one. The conditions used for the amide coupling step 8 were those described in the first part of Example 3 step 7. In step 10, purification was performed by chiral SFC using a Chiralpak AS-H column, 5 μm particle size, 25 cm×10 mm from Daicel Corporation (Mobile phase: 25% methanol (supplemented with 20 mM NH₃), 75% CO₂; System pressure: 100 bar) on a Minigram SFC instrument from Berger Instruments:

| Cmpd No. | Compound Name | LC/MS | NMR (shifts in ppm) |
|---|---|---|---|
| 30 | rel-4-((2R,3S,5S)-3-(3,4- | ESI-MS m/z calc. | ¹H NMR (500 MHz, |
| | difluoro-2-methoxyphenyl)-5- | 449.17624, found 450.3 | Methanol-d₄) δ 8.50 (dd, |
| | (2-methoxyethyl)-5- | (M + 1)⁺; 448.4 (M − 1)⁻; | J = 5.5, 0.6 Hz, 1H), 8.21 |
| | methyltetrahydrofuran-2- | Retention time: 2.80 | (dd, J = 2.2, 0.6 Hz, 1H), |
| | carboxamido)picolinamide | minutes | 7.92 (dd, J = 5.5, 2.2 Hz, |
| | (First eluting isomer by SFC on | | 1H), 7.23-7.17 (m, 1H), |
| | Chiralpak AS-H column, rt = | | 6.98 (ddd, J = 9.8, 8.9, |
| | 2.31 min) | | 7.5 Hz, 1H), 4.52 (d, J = |
| | | | 9.9 Hz, 1H), 4.00-3.91 |
| | | | (m, 1H), 3.88 (d, J = 2.0 |
| | | | Hz, 3H), 3.80-3.74 (m, |
| | | | 1H), 3.74-3.67 (m, 1H), |
| | | | 3.44 (s, 3H), 2.41 (dd, J = |
| | | | 12.5, 7.7 Hz, 1H), 2.17- |
| | | | 2.06 (m, 2H), 2.00-1.92 |
| | | | (m, 1H), 1.48 (s, 3H) |
| | | | ppm; amides NH and |
| | | | NH2 not observed. |
| 31 | rel-4-((2S,3R,5R)-3-(3,4- | ESI-MS m/z calc. | |
| | difluoro-2-methoxyphenyl)-5- | 449.17624, found 450.6 | |
| | (2-methoxyethyl)-5- | (M + 1)⁺; 448.7 (M − 1)⁻; | |
| | methyltetrahydrofuran-2- | Retention time: 2.86 | |
| | carboxamido)picolinamide | minutes | |
| | (Second eluting isomer by SFC | | |
| | on Chiralpak AS-H column,rt = | | |
| | 3.20 min) | | |

### Example 5

### rel-4-((2R,3S,5R)-3-(3,4-difluoro-2-(2-hydroxy-2-methylpropoxy)phenyl)-5-methyl-5-(trifluoromethyl)tetrahydrofuran-2-carboxamido) picolinamide (32)

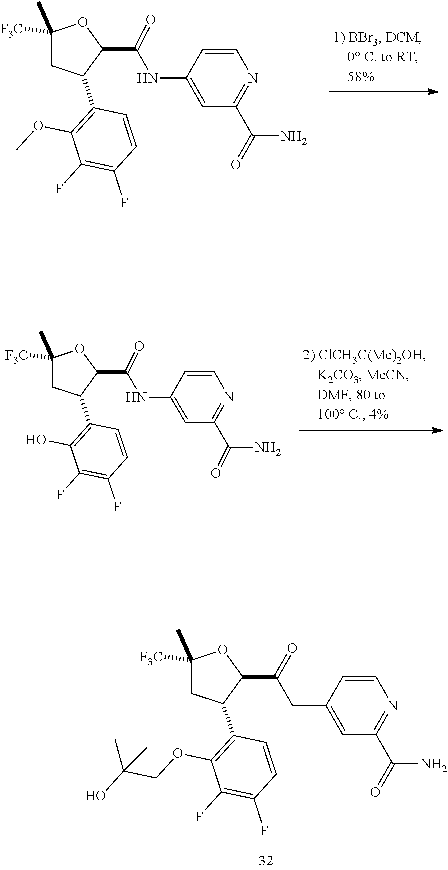 [SMILES: COc1c([C@@H]2C[C@](C)(C(F)(F)F)O[C@H]2C(=O)Nc2ccnc(C(N)=O)c2)ccc(F)c1F ; C[C@]1(C(F)(F)F)C[C@@H](c2ccc(F)c(F)c2O)[C@H](C(=O)Nc2ccnc(C(N)=O)c2)O1 ; CC(C)(O)COc1c([C@@H]2C[C@](C)(C(F)(F)F)O[C@H]2C(=O)Cc2ccnc(C(N)=O)c2)ccc(F)c1F]

### Step 1:

BBr₃ (2 mL, 1 M in DCM, 2.00 mmol) was added to a solution of rel-4-((2R,3S,5R)-3-(3,4-difluoro-2-methoxyphenyl)-5-methyl-5-(trifluoromethyl)tetrahydrofuran-2-carboxamido) picolinamide (Intermediate A-2, 749 mg, 1.61 mmol) in DCM (20 mL) at 0° C. The reaction mixture was allowed to warm up to ambient temperature over 24 h. Water (5 mL) and saturated sodium bicarbonate (10 mL) were added and the reaction was stirred for 30 min. The aqueous layer was extracted with DCM (3×15 mL). The combined organic extracts were dried over MgSO₄, filtered, and concentrated in vacuo. Purification by flash chromatography (40 g SiO₂, 0 to 100% EtOAc in heptane) gave rel-4-((2R,3S,5R)-3-(3,4-difluoro-2-hydroxyphenyl)-5-methyl-5-(trifluoromethyl)tetrahydrofuran-2-carboxamido) picolinamide (418.8 mg, 58%) as a white solid. ¹H NMR (500 MHZ, DMSO-d₆) δ 10.45 (s, 1H), 10.42 (s, 1H), 8.49 (d, J=5.5 Hz, 1H), 8.28 (d, J=1.9 Hz, 1H), 8.05 (d, J=2.3 Hz, 1H), 7.82 (dd, J=5.5, 2.2 Hz, 1H), 7.60 (d, J=2.4 Hz, 1H), 7.05 (ddd, J=8.4, 5.9, 2.0 Hz, 1H), 6.83 (q, J=8.7 Hz, 1H), 4.75 (d, J=10.1 Hz, 1H), 4.04-4.00 (m, 1H), 2.63 (t, J=12.3 Hz, 1H), 2.37 (dd, J=12.5, 8.0 Hz, 1H), 1.57 (s, 3H) ppm; ¹⁹F NMR (471 MHz, DMSO-d₆) δ −80.05, −139.44 (d, J=24.0 Hz), −159.72 (d, J=24.4 Hz) ppm. ESI-MS m/z calc. 445.1061, found 446.5 (M+1)⁺; 444.4 (M−1); Retention time: 0.73 minutes.

### Step 2:

A mixture of rel-4-((2R,3S,5R)-3-(3,4-difluoro-2-hydroxyphenyl)-5-methyl-5-(trifluoromethyl)tetrahydrofuran-2-carboxamido) picolinamide (80 mg, 0.18 mmol), K₂CO₃ (65 mg, 0.47 mmol) and 1-chloro-2-methylpropan-2-ol (280 μL, 2.73 mmol) in MeCN (2 mL) was heated in a sealed tube at 80° C. for 17 h. A further portion of 1-chloro-2-methylpropan-2-ol (280 μL, 2.73 mmol) was added and the reaction was further heated at 80° C. for 22 h. DMF (1 mL) was added and the reaction was heated at 80° C. for 24 h. A further portion of 1-chloro-2-methylpropan-2-ol (550 μL, 5.36 mmol) was added and the reaction was heated at 100° C. for 120 h. The mixture was cooled down to ambient temperature and partitioned between water (5 mL) and EtOAc (10 mL). The aqueous layer was separated and extracted with EtOAc (2×5 mL). The combined organic extracts were dried over MgSO₄, filtered, and concentrated in vacuo. Purification by reverse phase HPLC using a X-bridge C18 column (150×19 mm, 5 mm particle size) from Waters (Mobile phase: acetonitrile and water (supplemented with 0.1% ammonium hydroxide); Flow rate: 19 mL/min; sample dissolved in neat acetonitrile and injected at 1 mL/min) gave rel-4-((2R,3S,5R)-3-(3,4-difluoro-2-(2-hydroxy-2-methylpropoxy)phenyl)-5-methyl-5-(trifluoromethyl)tetrahydrofuran-2-carboxamido) picolinamide (32, 3.7 mg, 4%) as an off-white solid. 1H NMR (500 MHz, DMSO-d₆) δ 10.41 (s, 1H), 8.48 (d, J=5.5 Hz, 1H), 8.26 (d, J=2.3 Hz, 1H), 8.05 (s, 1H), 7.84 (dd, J=5.5, 2.2 Hz, 1H), 7.60 (s, 1H), 7.29-7.25 (m, 1H), 7.23-7.18 (m, 1H), 4.68 (d, J=10.3 Hz, 1H), 4.62 (s, 1H), 4.28-4.22 (m, 1H), 3.77 (q, J=9.0 Hz, 2H), 2.56-2.52 (m, 1H), 2.30 (t, J=12.4 Hz, 1H), 1.56 (s, 3H), 1.19 (s, 3H), 1.15 (s, 3H) ppm; ¹⁹F NMR (471 MHz, DMSO-d₆) δ −80.02, −138.29 (d, J=22.3 Hz), −154.19 (d, J=22.1 Hz). ESI-MS m/z calc. 517.16364, found 518.5 (M+1)⁺; 516.6 (M−1); Retention time: 2.9 minutes.

The following compounds were made using the method described in Example 5, except that different alkylating agents were used in step 2 in place of 1-chloro-2-methylpropan-2-ol:

| Cmpd No. | Compound Name | LC/MS | NMR (shifts in ppm) |
|---|---|---|---|
| 33 | rel-4-((2R,3S,5R)-3-(3,4- | ESI-MS m/z calc. | ¹H NMR (500 MHz, |
| | difluoro-2-(2- | 489.13232, found 490.4 | DMSO-d₆) δ 10.41 (s, |
| | hydroxyethoxy)phenyl)-5- | (M + 1)⁺; 488.6 (M − 1)⁻; | 1H), 8.49 (d, J = 5.5 Hz, |
| | methyl-5- | Retention time: 2.69 | 1H), 8.28 (d, J = 2.1 Hz, |
| | (trifluoromethyl)tetrahydrofuran- | minutes | 1H), 8.08 (d, J = 2.8 Hz, |
| | 2-carboxamido)picolinamide | | 1H), 7.84 (dd, J = 5.5, 2.2 |
| | | | Hz, 1H), 7.63 (d, J = 2.8 |
| | | | Hz, 1H), 7.25-7.16 (m, |
| | | | 2H), 4.91 (t, J = 5.0 Hz, |
| | | | 1H), 4.71 (d, J = 10.3 Hz, |
| | | | 1H), 4.23-4.17 (m, 1H), |
| | | | 4.07 (t, J = 4.7 Hz, 2H), |
| | | | 3.70-3.58 (m, 2H), 2.38 |
| | | | (t, J = 12.3 Hz, 1H), 1.57 |
| | | | (s, 3H) ppm; alcohol OH |
| | | | not observed. |
| 34 | rel-4-((2R,3S,5R)-3-(3,4- | ESI-MS m/z calc. | ¹H NMR (500 MHz, |
| | difluoro-2-(2- | 503.14795, found 504.5 | DMSO-d₆) δ 10.41 (s, |
| | methoxyethoxy)phenyl)-5- | (M + 1)⁺; 502.6 (M − 1)⁻; | 1H), 8.49 (d, J = 5.5 Hz, |
| | methyl-5- | Retention time: 3.03 | 1H), 8.29 (d, J = 2.1 Hz, |
| | (trifluoromethyl)tetrahydrofuran- | minutes | 1H), 8.08 (d, J = 2.8 Hz, |
| | 2-carboxamido)picolinamide | | 1H), 7.84 (dd, J = 5.5, 2.2 |
| | | | Hz, 1H), 7.63 (d, J = 2.8 |
| | | | Hz, 1H), 7.26-7.17 (m, |
| | | | 2H), 4.69 (d, J = 10.3 Hz, |
| | | | 1H), 4.20-4.12 (m, 3H), |
| | | | 3.57-3.55 (m, 2H), 3.23 |
| | | | (s, 3H), 2.47-2.38 (m, |
| | | | 2H), 1.58 (s, 3H) ppm. |
| 35 | rel-4-((2R,3S,5R)-3-(3,4- | ESI-MS m/z calc. | ¹H NMR (500 MHz, |
| | difluoro-2-(3- | 503.14795, found 504.4 | DMSO-d₆) δ 10.41 (s, |
| | hydroxypropoxy)phenyl)-5- | (M + 1)⁺; 502.6 (M − 1)⁻; | 1H), 8.49 (d, J = 5.5 Hz, |
| | methyl-5- | Retention time: 2.74 | 1H), 8.26 (d, J = 2.0 Hz, |
| | (trifluoromethyl)tetrahydrofuran- | minutes | 1H), 8.06 (d, J = 2.0 Hz, |
| | 2-carboxamido)picolinamide | | 1H), 7.83 (dd, J = 5.5, 2.2 |
| | | | Hz, 1H), 7.61-7.60 (m, |
| | | | 1H), 7.25-7.16 (m, 2H), |
| | | | 4.65 (d, J = 10.1 Hz, 1H), |
| | | | 4.50 (t, J = 5.1 Hz, 1H), |
| | | | 4.16-4.04 (m, 3H), 3.56- |
| | | | 3.46 (m, 2H), 2.47- |
| | | | 2.41 (m, 2H), 1.87-1.77 |
| | | | (m, 2H), 1.58 (s, 3H) |
| | | | ppm. |

The following compounds were made using the method described in Example 5, except that rac-4-((2R,3S,5R)-3-(4-fluoro-2-methoxy-3-methylphenyl)-5-methyl-5-(trifluoromethyl)tetrahydrofuran-2-carboxamido) picolinamide (Intermediate H) was used as the starting material in step 1. In step 2, 2-chloroethan-1-ol was used as the alkylating agent in place of 1-chloro-2-methylpropan-2-ol. At the end of the synthesis, the enantiomers were further separated by chiral SFC using a Chiralpak IG column, 5 mm particle size, 25 cm×10 mm from Daicel Corporation (Mobile phase: 12% methanol (supplemented with 20 mM NH₃), 88% CO₂; System pressure: 134 bar) on a Minigram SFC instrument from Berger Instruments:

| Cmpd No. | Compound Name | LC/MS | NMR (shifts in ppm) |
|---|---|---|---|
| 36 | rel-4-((2S,3R,5S)-3-(4-fluoro-2- | ESI-MS m/z calc. | ¹H NMR (500 MHz, |
| | (2-hydroxyethoxy)-3- | 485.15738, found 486.4 | DMSO-d₆) δ 10.41 (s, |
| | methylphenyl)-5-methyl-5- | (M + 1)⁺; 484.5 (M − 1)⁻; | 1H), 8.48 (d, J = 5.5 Hz, |
| | (trifluoromethyl)tetrahydrofuran- | Retention time: 2.75 | 1H), 8.25 (d, J = 2.1 Hz, |
| | 2-carboxamido)picolinamide | minutes | 1H), 8.05 (d, J = 2.8 Hz, |
| | (First eluting isomer by SFC on | | 1H), 7.80 (dd, J = 5.5, 2.2 |
| | Chiralpak IG column, rt = 6.45 | | Hz, 1H), 7.59 (d, J = 2.8 |
| | min) | | Hz, 1H), 7.28 (dd, J = |
| | | | 8.7, 6.5 Hz, 1H), 7.01 (t, J = |
| | | | 8.8 Hz, 1H), 4.91 (t, J = |
| | | | 5.4 Hz, 1H), 4.66 (d, J = |
| | | | 10.3 Hz, 1H), 4.29-4.19 |
| | | | (m, 1H), 3.78-3.72 (m, |
| | | | 2H), 3.72-3.59 (m, 2H), |
| | | | 2.53-2.51 (m, 1H), 2.25 |
| | | | (t, J = 12.3 Hz, 1H), 2.12 |
| | | | (d, J = 2.0 Hz, 3H), 1.57 |
| | | | (s, 3H) ppm. |
| 37 | rel-4-((2R,3S,5R)-3-(4-fluoro- | ESI-MS m/z calc. | |
| | 2-(2-hydroxyethoxy)-3- | 485.15738, found 486.4 | |
| | methylphenyl)-5-methyl-5- | (M + 1)⁺; 484.5 (M − 1)⁻; | |
| | (trifluoromethyl)tetrahydrofuran- | Retention time: 2.75 | |
| | 2-carboxamido)picolinamide | minutes | |
| | (Second eluting isomer by SFC | | |
| | on Chiralpak IG column, rt = | | |
| | 7.04 min) | | |

### Example 6

### 4-((2R,3S,4S,5R)-3-(3,4-difluoro-2-(2-hydroxyethoxy)phenyl)-4,5-dimethyl-5-(trifluoromethyl)tetrahydrofuran-2-carboxamido)-N-methylpicolinamide (38)

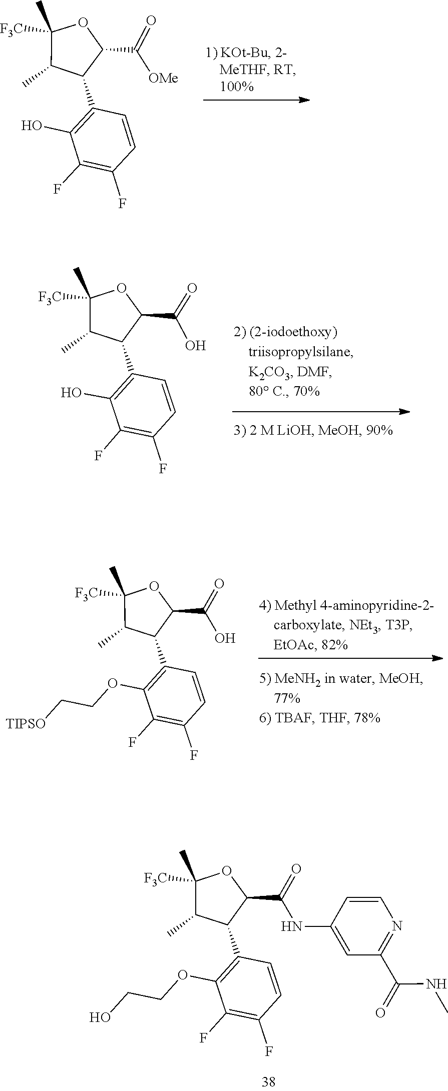

### Step 1:

Potassium tert-butoxide (1.90 g, 16.93 mmol) was added portionwise to a solution of methyl(2S,3S,4S,5R)-3-(3,4-difluoro-2-hydroxyphenyl)-4,5-dimethyl-5-(trifluoromethyl)tetrahydrofuran-2-carboxylate (Product of Example 2, Step 2, 2.00 g, 5.645 mmol) in 2-MeTHF (40 mL). The reaction mixture was stirred at ambient temperature overnight. The mixture was quenched by addition of 1M HCl (60 mL) and diluted with DCM (100 mL). The aqueous layer was separated and extracted with DCM (2×50 mL). The organic extracts were combined, washed with brine (50 mL), dried over MgSO₄, filtered, and concentrated in vacuo to give (2R,3S,4S,5R)-3-(3,4-difluoro-2-hydroxyphenyl)-4,5-dimethyl-5-(trifluoromethyl)tetrahydrofuran-2-carboxylic acid (1.964 g, 100%) as a yellow foam. ¹H NMR (500 MHz, Chloroform-d) δ 6.95-6.90 (m, 1H), 6.73-6.66 (m, 1H), 4.98 (d, J=10.3 Hz, 1H), 4.17 (dd, J=10.3, 7.8 Hz, 1H), 2.86-2.78 (m, 1H), 1.60 (s, 3H), 0.79-0.75 (m, 3H) ppm; alcohol and acid OH not observed.

### Step 2:

2-Iodoethoxy (triisopropyl) silane (Intermediate R-1) (1.08 mL, 3.553 mmol) was added to a stirred mixture of (2R,3S,4S,5R)-3-(3,4-difluoro-2-hydroxyphenyl)-4,5-dimethyl-5-(trifluoromethyl)tetrahydrofuran-2-carboxylic acid (5.47 g, 11.41 mmol) and potassium carbonate (6.3 g, 45.58 mmol) in DMF (25 mL). The reaction mixture was stirred in a sealed vial at 80° C. for 24 h. The reaction mixture was partitioned between MTBE (50 mL) and water (50 mL). The aqueous was extracted with MTBE (50 mL). The combined organic extracts were washed with brine (25 mL), passed through a phase separation cartridge, and concentrated in vacuo. Purification by flash chromatography (60 g SiO₂, 0 to 100% EtOAc in heptane) gave 2-((triisopropylsilyl)oxy)ethyl(2R,3S,4S,5R)-3-(3,4-difluoro-2-(2-((triisopropylsilyl)oxy)ethoxy)phenyl)-4,5-dimethyl-5-(trifluoromethyl)tetrahydrofuran-2-carboxylate (9.85 g, 70%), as an oil. ¹H NMR (500 MHZ, DMSO-d₆) δ 7.18 (dd, J=8.6, 6.2 Hz, 1H), 7.10 (q, J=8.9 Hz, 1H), 5.11 (d, J=10.3 Hz, 1H), 4.24-4.16 (m, 3H), 4.14-4.10 (m, 2H), 3.99 (t, J=4.7 Hz, 2H), 3.75 (dd, J=5.6, 4.0 Hz, 2H), 2.73 (p, J=7.6 Hz, 1H), 1.53 (s, 3H), 1.07-0.95 (m, 42H), 0.73-0.68 (m, 3H) ppm.

### Step 3:

2 M LiOH (2.75 mL, 5.500 mmol) was added to a solution of 2-((triisopropylsilyl)oxy)ethyl(2R,3S,4S,5R)-3-(3,4-difluoro-2-(2-((triisopropylsilyl)oxy)ethoxy)phenyl)-4,5-dimethyl-5-(trifluoromethyl)tetrahydrofuran-2-carboxylate (2.73 g, 3.684 mmol) in MeOH (25 mL). The reaction mixture was stirred at room temperature overnight before being concentrated in vacuo. The mixture was partitioned between 1 M HCl (10 mL) and MTBE (10 mL). The aqueous phase was separated and extracted with MTBE (10 mL). The combined organic extracts were washed with brine (5 mL), passed through a phase separation cartridge, and concentrated in vacuo. Purification by flash chromatography (24 g SiO₂, 0 to 20% EtOAc in heptane) gave (2R,3S,4S,5R)-3-(3,4-difluoro-2-(2-((triisopropylsilyl)oxy)ethoxy)phenyl)-4,5-dimethyl-5-(trifluoromethyl)tetrahydrofuran-2-carboxylic acid (1.79 g, 90%), as a colourless oil. ¹H NMR (500 MHZ, DMSO-d₆) δ 12.98 (s, 1H), 7.19 (ddd, J=7.7, 5.9, 1.7 Hz, 1H), 7.16-7.08 (m, 1H), 5.00 (d, J=10.5 Hz, 1H), 4.27-4.11 (m, 3H), 4.00 (t, J=4.5 Hz, 2H), 2.71 (p, J=7.5 Hz, 1H), 1.52 (s, 3H), 1.03 (d, J=6.3 Hz, 21H), 0.69 (dd, J=7.6, 2.4 Hz, 3H) ppm. 1° F. NMR (471 MHz, DMSO-d₆) δ −73.65, −138.38 (d, J=21.1 Hz), −154.75 (d, J=20.9 Hz) ppm. ESI-MS m/z calc. 540.23303, found 541.1 (M+1)⁺; 539.2 (M−1); Retention time: 0.91 minutes.

### Step 4:

T3P (770 μL, 2.589 mmol) was added to a solution of (2R,3S,4S,5R)-3-(3,4-difluoro-2-(2-((triisopropylsilyl)oxy)ethoxy)phenyl)-4,5-dimethyl-5-(trifluoromethyl)tetrahydrofuran-2-carboxylic acid (350 mg, 0.6474 mmol), methyl 4-aminopyridine-2-carboxylate (225 mg, 1.479 mmol) and Et₃N (361 μL, 2.590 mmol) in ethyl acetate (3 mL). The mixture was stirred at ambient temperature for 4 h. The mixture was partitioned between ethyl acetate (30 ml) and water (30 ml). The aqeuous layer was separated and extracted with EtOAc (50 mL). The combined organic extracts were washed with brine (20 mL), dried over MgSO₄, filtered and concentrated in vacuo. Purification by flash chromatography (12 g SiO₂, 0 to 100% EtOAc in Hexanes) gave methyl 4-((2R,3S,4S,5R)-3-(3,4-difluoro-2-(2-((triisopropylsilyl)oxy)ethoxy)phenyl)-4,5-dimethyl-5-(trifluoromethyl)tetrahydrofuran-2-carboxamido) picolinate (360 mg, 82%). ESI-MS m/z calc. 674.28107, found 67.0 (M+1)⁺; 673.9 (M−1); Retention time: 1.36 minutes.

### Step 5:

A solution of methyl 4-((2R,3S,4S,5R)-3-(3,4-difluoro-2-(2-((triisopropylsilyl)oxy)ethoxy)phenyl)-4,5-dimethyl-5-(trifluoromethyl)tetrahydrofuran-2-carboxamido) picolinate (370 mg, 0.5483 mmol) in MeOH (3 mL) was treated with MeNH₂ (425 μL of 40% w/v in water, 5.474 mmol). The mixture was stirred for 40 min at ambient temperature before being concentrated in vacuo. Purification by reverse phase HPLC using a X-bridge C18 column (150×19 mm, 5 mm particle size) from Waters (Gradient: 47.4% to 94.7% acetonitrile in water (supplemented with 0.1% ammonium hydroxide) over 9 minutes; Flow rate: 19 mL/min; sample dissolved in neat acetonitrile and injected at 1 mL/min) gave 4-((2R,3S,4S,5R)-3-(3,4-difluoro-2-(2-((triisopropylsilyl)oxy)ethoxy)phenyl)-4,5-dimethyl-5-(trifluoromethyl)tetrahydrofuran-2-carboxamido)-N-methylpicolinamide (290 mg, 77%). ESI-MS m/z calc. 673.29706, found 675.0 (M+1)⁺; Retention time: 3.73 minutes.

### Step 6:

TBAF (542 μL of 1 M, 0.5420 mmol) was added to a solution of 4-((2R,3S,4S,5R)-3-(3,4-difluoro-2-(2-((triisopropylsilyl)oxy)ethoxy)phenyl)-4,5-dimethyl-5-(trifluoromethyl)tetrahydrofuran-2-carboxamido)-N-methylpicolinamide (100 mg, 0.1465 mmol) in THF (2 mL). The reaction mixture was stirred at ambient temperature for 2 h before being concentrated in vacuo. Purification by reverse phase preparative HPLC gave 4-((2R,3S,4S,5R)-3-(3,4-difluoro-2-(2-hydroxyethoxy)phenyl)-4,5-dimethyl-5-(trifluoromethyl)tetrahydrofuran-2-carboxamido)-N-methylpicolinamide (38, 59 mg, 78%). ¹H NMR (500 MHz, DMSO-d₆) δ 10.72 (s, 1H), 8.73 (d, J=5.0 Hz, 1H), 8.47 (d, J=5.5 Hz, 1H), 8.30-8.15 (m, 1H), 7.83 (dd, J=5.5, 2.1 Hz, 1H), 7.25-7.04 (m, 2H), 5.10 (d, J=10.7 Hz, 1H), 4.41 (dd, J=10.7, 7.3 Hz, 1H), 4.18-4.06 (m, 2H), 3.70 (t, J=4.6 Hz, 2H), 2.91 (p, J=7.4 Hz, 1H), 2.80 (d, J=4.8 Hz, 3H), 1.61 (s, 3H), 1.31 (h, J=7.3 Hz, 1H), 0.77-0.64 (m, 3H) ppm. ESI-MS m/z calc. 517.16364, found 518.7 (M+1)⁺; 516.7 (M−1); Retention time: 2.97 minutes.

The following compound was made using the method described in Example 6, except that in step 2, 4-(2-chloroethyl) morpholine was used as the alkylating agent with Cs₂CO₃ as the base. The conditions used for the aminolysis step 5 were those described in Example 2 step 9. The deprotection step 6 was not required:

The following compounds were made using the method described in Example 6, except that the conditions used in step 1 were those described in Example 2 step 4 with only MeOH as the solvent. The conditions used in step 2 were those described in Example 10 step 1 using a different alcohol. Step 3 was carried out in THE as the solvent. Step 4 was carried out at ambient temperature until reaction completion in the presence of HATU as the activating agent, Et₃N as the base in DMF as the solvent, conditions well known in the art. Step 6 was not required. In Step 5, in the case of compounds 40 and 42, a 7 M methanolic ammonia solution was used in place of MeNH₂ in a mixture of water and MeOH:

| Cmpd No. | Compound Name | LC/MS | NMR (shifts in ppm) |
|---|---|---|---|
| 40 | 4-((2R,3S,4S,5R)-3-(2-((S)-2- | ESI-MS m/z calc. | ¹H NMR (400 MHz, |
| | (2-oxa-6-azaspiro[3.3]heptan-6- | 598.2215, found 599.3 | Chloroform-d) δ 8.71 (s, |
| | yl)propoxy)-3,4- | (M + 1)⁺; 597.2 (M − 1)⁻; | 1H), 8.47 (d, J = 5.5 Hz, |
| | difluorophenyl)-4,5-dimethyl-5- | Retention time: 2.37 | 1H), 8.12 (s, 1H), 7.94 (s, |
| | (trifluoromethyl)tetrahydrofuran- | minutes | 1H), 7.82 (s, 1H), 7.07 (t, |
| | 2-carboxamido)picolinamide | | J = 6.6 Hz, 1H), 6.93 (q, J = |
| | | | 8.5 Hz, 1H), 5.57 (s, |
| | | | 1H), 5.01 (d, J = 9.6 Hz, |
| | | | 1H), 4.63 (s, 4H), 4.13 (d, |
| | | | J = 45.3 Hz, 2H), 3.79 (s, |
| | | | 1H), 3.36 (s, 4H), 2.75 (s, |
| | | | 1H), 2.48 (s, 1H), 1.68 (s, |
| | | | 3H), 1.05 (s, 3H), 0.78 |
| | | | (dd, J = 7.6, 2.1 Hz, 3H) |
| | | | ppm. |
| 41 | 4-((2R,3S,4S,5R)-3-(2-((R)-2- | ESI-MS m/z calc. | ¹H NMR (400 MHz, |
| | (2-oxa-6-azaspiro[3.3]heptan-6- | 612.2371, found 613.3 | Chloroform-d) δ 8.59 (s, |
| | yl)propoxy)-3,4- | (M + 1)⁺; 611.2 (M − 1)⁻; | 1H), 8.42 (d, J = 5.5 Hz, |

| Cmpd No. | Compound Name | LC/MS | NMR (shifts in ppm) |
|---|---|---|---|
| | difluorophenyl)-4,5-dimethyl-5- | Retention time: 2.41 | 1H), 8.12 (d, J = 4.6 Hz, |
| | (trifluoromethyl)tetrahydrofuran- | minutes | 1H), 8.00 (d, J = 5.5 Hz, |
| | 2-carboxamido)-N- | | 1H), 7.86 (s, 1H), 7.07 (t, |
| | methylpicolinamide | | J = 6.4 Hz, 1H), 6.93 (t, J = |
| | | | 8.5 Hz, 1H), 4.98 (d, J = |
| | | | 11.0 Hz, 1H), 4.68 (s, |
| | | | 4H), 4.07 (t, J = 9.4 Hz, |
| | | | 1H), 3.92 (s, 2H), 3.35 (s, |
| | | | 4H), 3.03 (d, J = 5.0 Hz, |
| | | | 3H), 2.86 (t, J = 7.3 Hz, |
| | | | 1H), 2.43 (d, J = 5.0 Hz, |
| | | | 1H), 1.66 (s, 3H), 1.05 (d, |
| | | | J = 6.4 Hz, 3H), 0.78 (dd, |
| | | | J = 7.3, 1.8 Hz, 3H) ppm. |
| 42 | 4-((2R,3S,4S,5R)-3-(2-((R)-2- | ESI-MS m/z calc. | ¹H NMR (400 MHz, |
| | (2-oxa-6-azaspiro[3.3]heptan-6- | 598.2215, found 599.3 | Chloroform-d) δ 8.66 (s, |
| | yl)propoxy)-3,4- | (M + 1)⁺; 597.2 (M − 1)⁻; | 1H), 8.46 (d, J = 5.5 Hz, |
| | difluorophenyl)-4,5-dimethyl-5- | Retention time: 2.30 | 1H), 8.12 (s, 1H), 7.94 (s, |
| | (trifluoromethyl)tetrahydrofuran- | minutes | 1H), 7.82 (d, J = 3.7 Hz, |
| | 2-carboxamido)picolinamide | | 1H), 7.08 (t, J = 6.2 Hz, |
| | | | 1H), 6.92 (q, J = 8.5 Hz, |
| | | | 1H), 5.58 (s, 1H), 4.99 (d, |
| | | | J = 11.4 Hz, 1H), 4.69 (s, |
| | | | 4H), 4.08 (s, 1H), 3.93 (s, |
| | | | 2H), 3.36 (s, 4H), 2.87 (t, |
| | | | J = 7.6 Hz, 1H), 2.44 (s, |
| | | | 1H), 1.67 (s, 3H), 1.05 (d, |
| | | | J = 6.4 Hz, 3H), 0.79 (dd, |
| | | | J = 7.3, 1.8 Hz, 3H) ppm. |
| 43 | 4-((2R,3S,4S,5R)-3-(2-((S)-2- | ESI-MS m/z calc. | ¹H NMR (400 MHz, |
| | (2-oxa-6-azaspiro[3.3]heptan-6- | 612.2371, found 613.28 | Chloroform-d) δ 8.71 (s, |
| | yl)propoxy)-3,4- | (M + 1)⁺; 611.2 (M − 1)⁻; | 1H), 8.43 (d, J = 5.5 Hz, |
| | difluorophenyl)-4,5-dimethyl-5- | Retention time: 2.47 | 1H), 8.12 (s, 1H), 8.00 (d, |
| | (trifluoromethyl)tetrahydrofuran- | minutes | J = 4.6 Hz, 1H), 7.87 (s, |
| | 2-carboxamido)-N- | | 1H), 7.07 (t, J = 6.4 Hz, |
| | methylpicolinamide | | 1H), 6.92 (q, J = 8.5 Hz, |
| | | | 1H), 5.00 (d, J = 9.6 Hz, |
| | | | 1H), 4.62 (s, 4H), 4.19 (s, |
| | | | 1H), 4.09 (s, 1H), 3.79 (s, |
| | | | 1H), 3.36 (s, 4H), 3.02 (d, |
| | | | J = 5.0 Hz, 3H), 2.75 (s, |
| | | | 1H), 2.48 (s, 1H), 1.68 (s, |
| | | | 3H), 1.05 (s, 3H), 0.79- |
| | | | 0.77 (m, 3H) ppm. |

### Example 7

### 4-((2R,3S,4S,5R)-3-(2-(2-(dimethylamino)ethoxy)-3,4-difluorophenyl)-4,5-dimethyl-5-(trifluoromethyl)tetrahydrofuran-2-carboxamido) picolinamide (44)

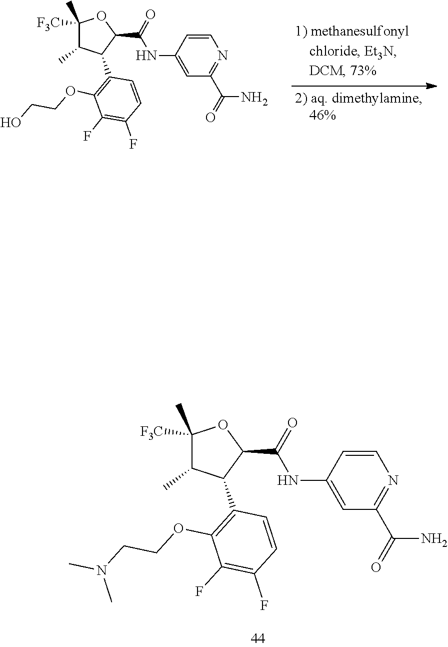 [SMILES: C[C@H]1[C@@H](c2ccc(F)c(F)c2OCCO)[C@H](C(=O)Nc2ccnc(C(N)=O)c2)O[C@@]1(C)C(F)(F)F ; C[C@H]1[C@@H](c2ccc(F)c(F)c2OCCN(C)C)[C@H](C(=O)Nc2ccnc(C(N)=O)c2)O[C@@]1(C)C(F)(F)F]

### Step 1 and 2:

Et₃N (50 μL, 0.3587 mmol) and methanesulfonyl chloride (20 μL, 0.2584 mmol) were successively added to a solution of 4-((2R,3S,4S,5R)-3-(3,4-difluoro-2-(2-hydroxyethoxy)phenyl)-4,5-dimethyl-5-(trifluoromethyl)tetrahydrofuran-2-carboxamido) picolinamide (5) (100 mg, 0.199 mmol) in DCM (3 mL). The reaction mixture was stirred for 20 min. The mixture was partitioned between MTBE (10 ml) and water (10 ml). The aqeuous layer was separated and extracted with MTBE (5 mL). The combined organic extracts were washed with brine (1×3 mL), dried over MgSO₄, filtered, and concentrated in vacuo to give 2-(6-((2R,3S,4S,5R)-2-((2-carbamoylpyridin-4-yl) carbamoyl)-4,5-dimethyl-5-(trifluoromethyl)tetrahydrofuran-3-yl)-2,3-difluorophenoxy)ethyl methanesulfonate (100 mg, 73%) as an oil. ESI-MS m/z calc. 581.1255, found 582.7 (M+1)⁺; 580.6 (M−1); Retention time: 0.88 minutes.

N-Methylmethanamine (500 μL of 40% w/w in water, 3.993 mmol) was added to 2-(6-((2R,3S,4S,5R)-2-((2-carbamoylpyridin-4-yl) carbamoyl)-4,5-dimethyl-5-(trifluoromethyl)tetrahydrofuran-3-yl)-2,3-difluorophenoxy)ethyl methanesulfonate (100 mg). The mixture was stirred at ambient temperature for 2 h. The reaction mixture was partitioned between MTBE (20 ml) and water (20 ml). The aqueous layer was separated and extracted with MTBE (10 mL). The combined organic extracts were washed with brine (1×10 mL), dried over MgSO₄, filtered, and concentrated in vacuo. Purification by reverse phase preparative HPLC gave 4-((2R,3S,4S,5R)-3-(2-(2-(dimethylamino)ethoxy)-3,4-difluorophenyl)-4,5-dimethyl-5-(trifluoromethyl)tetrahydrofuran-2-carboxamido) picolinamide (44, 50 mg, 46%). ¹H NMR (500 MHz, DMSO-d₆) δ 10.67 (s, 1H), 8.49 (d, J=5.5 Hz, 1H), 8.30 (d, J=2.2 Hz, 1H), 8.09 (d, J=2.8 Hz, 1H), 7.86 (dd, J=5.5, 2.2 Hz, 1H), 7.64 (d, J=2.8 Hz, 1H), 7.17 (dd, J=8.6, 4.4 Hz, 2H), 5.13 (d, J=10.7 Hz, 1H), 4.44 (dd, J=10.7, 7.3 Hz, 1H), 4.27 (dt, J=10.7, 5.1 Hz, 1H), 4.11 (dt, J=10.9, 5.2 Hz, 1H), 2.87 (p, J=7.4 Hz, 1H), 2.55 (ddd, J=8.8, 6.3, 4.0 Hz, 1H), 2.14 (s, 6H), 2.04-1.96 (m, 1H), 1.63 (s, 3H), 0.70 (d, J=7.3 Hz, 3H) ppm. ESI-MS m/z calc. 530.19525, found 531.7 (M+1)⁺; 529.7 (M−1); Retention time: 3.22 minutes.

The following compounds were made using the method described in Example 7, except that the conditions used in step 2 were those described in Example 8 step 5, using different amines:

| Cmpd No. | Compound Name | LC/MS | NMR (shifts in ppm) |
|---|---|---|---|
| 45 | 4-((2R,3S,4S,5R)-3-(2-(2-(1H- | ESI-MS m/z calc. | ¹H NMR (500 MHz, |
| | imidazol-1-yl)ethoxy)-3,4- | 553.17487, found 554.4 | DMSO-d₆) δ 10.63 (s, |
| | difluorophenyl)-4,5-dimethyl-5- | (M + 1)⁺; 552.3 (M − 1)⁻; | 1H), 8.50 (d, J = 5.5 Hz, |
| | (trifluoromethyl)tetrahydrofuran- | Retention time: 2.84 | 1H), 8.31 (d, J = 2.2 Hz, |
| | 2-carboxamido)picolinamide | minutes | 1H), 8.06 (s, 1H), 7.85 |
| | | | (dd, J = 5.5, 2.2 Hz, 1H), |
| | | | 7.71 (t, J = 1.1 Hz, 1H), |
| | | | 7.62 (s, 1H), 7.23 (t, J = |
| | | | 1.2 Hz, 1H), 7.21-7.11 |
| | | | (m, 2H), 6.86 (t, J = 1.1 |
| | | | Hz, 1H), 5.06 (d, J = 9.9 |
| | | | Hz, 1H), 4.47 (td, J = 8.2, |
| | | | 5.4 Hz, 1H), 4.43-4.37 |
| | | | (m, 2H), 3.96 (dd, J = 9.9, |
| | | | 7.9 Hz, 1H), 2.43 (q, J = |
| | | | 7.6 Hz, 1H), 2.02-1.96 |
| | | | (m, 1H), 1.51 (s, 3H), |
| | | | 0.66-0.60 (m, 3H) ppm. |
| 46 | rel-4-((2R*,3S*,4S*,5R*)-3-(2- | ESI-MS m/z calc. | ¹H NMR (500 MHz, |
| | (2-((1S,4S)-2-oxa-5- | 584.2058, found 585.3 | DMSO-d₆) δ 10.69 (d, J = |
| | azabicyclo[2.2.1]heptan-5- | (M + 1)⁺; 583.3 (M − 1)⁻; | 13.2 Hz, 1H), 8.50 (dd, J = |
| | yl)ethoxy)-3,4-difluorophenyl)- | Retention time: 3.02 | 5.6, 1.3 Hz, 1H), 8.30 |
| | 4,5-dimethyl-5- | minutes | (d, J = 2.4 Hz, 1H), 8.06 |
| | (trifluoromethyl)tetrahydrofuran- | | (d, J = 2.7 Hz, 1H), 7.87- |
| | 2-carboxamido)picolinamide | | 7.79 (m, 1H), 7.61 (d, J = |
| | | | 2.8 Hz, 1H), 7.17 (dd, J = |
| | | | 8.7, 4.8 Hz, 2H), 5.16- |
| | | | 5.06 (m, 1H), 4.50 (d, J = |
| | | | 11.4 Hz, 1H), 4.43-4.25 |
| | | | (m, 4H), 4.18 (q, J = 6.4 |
| | | | Hz, 1H), 3.64 (q, J = 7.6 |
| | | | Hz, 1H), 3.60-3.52 (m, |
| | | | 1H), 3.46-3.37 (m, 1H), |
| | | | 3.21 (d, J = 5.1 Hz, 1H), |
| | | | 2.94-2.77 (m, 2H), 2.45- |
| | | | 2.39 (m, 1H), 1.73-1.65 |
| | | | (m, 1H), 1.62 (d, J = 16.0 |
| | | | Hz, 3H), 0.71 (s, 3H) |
| | | | ppm. |
| 47 | rel-4-((2R*,3S*,4S*,5R*)-3-(2- | ESI-MS m/z calc. | ¹H NMR (500 MHz, |
| | (2-((1R,4R)-2-oxa-5- | 584.2058, found 585.3 | DMSO-d₆) δ 10.65 (s, |
| | azabicyclo[2.2.1]heptan-5- | (M + 1)⁺; 583.3 (M − 1)⁻; | 1H), 8.50 (d, J = 5.5 Hz, |
| | yl)ethoxy)-3,4-difluorophenyl)- | Retention time: 3.04 | 1H), 8.30 (d, J = 2.2 Hz, |
| | 4,5-dimethyl-5- | minutes | 1H), 8.06 (d, J = 2.8 Hz, |
| | (trifluoromethyl)tetrahydrofuran- | | 1H), 7.85 (dd, J = 5.5, 2.2 |
| | 2-carboxamido)picolinamide | | Hz, 1H), 7.61 (d, J = 2.7 |
| | | | Hz, 1H), 7.16 (dd, J = |
| | | | 13.4, 6.3 Hz, 2H), 5.14 |
| | | | (d, J = 10.5 Hz, 1H), 4.42 |
| | | | (dd, J = 10.4, 7.6 Hz, |
| | | | 1H), 4.34-4.26 (m, 1H), |
| | | | 4.15 (s, 1H), 4.04 (d, J = |
| | | | 5.9 Hz, 1H), 3.83 (d, J = |
| | | | 7.6 Hz, 1H), 3.49-3.40 |
| | | | (m, 2H), 3.29 (s, 1H), |
| | | | 2.84 (dq, J = 22.5, 7.5, |
| | | | 6.7 Hz, 3H), 2.78-2.72 |
| | | | (m, 1H), 2.32 (d, J = 9.8 |
| | | | Hz, 1H), 1.65 (s, 3H), |
| | | | 1.50 (d, J = 9.5 Hz, 1H), |
| | | | 0.71 (d, J = 7.2 Hz, 3H) |
| | | | ppm. |
| 48 | 4-((2R,3S,4S,5R)-3-(3,4- | ESI-MS m/z calc. | ¹H NMR (500 MHz, |
| | difluoro-2-(2-(3-methoxy-3- | 586.22144, found 587.3 | DMSO-d₆) δ 10.67 (s, |
| | methylazetidin-1- | (M + 1)⁺; 585.2 (M − 1)⁻; | 1H), 8.46 (s, 1H), 8.27 (s, |
| | yl)ethoxy)phenyl)-4,5- | Retention time: 3.20 | 1H), 8.03 (s, 1H), 7.82 (s, |
| | dimethyl-5- | minutes. | 1H), 7.57 (s, 1H), 7.16 |
| | (trifluoromethyl)tetrahydrofuran- | | (dd, J = 14.3, 6.6 Hz, |
| | 2-carboxamido)picolinamide | | 2H), 5.10 (s, 1H), 4.33 (d, |
| | | | J = 25.1 Hz, 1H), 4.14 |
| | | | (dd, J = 10.6, 5.2 Hz, |
| | | | 2H), 4.07-4.00 (m, 2H), |
| | | | 3.12 (dd, J = 13.0, 8.1 Hz, |
| | | | 2H), 3.02 (s, 3H), 2.98- |
| | | | 2.83 (m, 2H), 2.76 (s, |
| | | | 1H), 1.66 (s, 3H), 1.28 (s, |
| | | | 3H), 0.74-0.68 (m, 3H) |
| | | | ppm. |
| 49 | 4-((2R,3S,4S,5R)-3-(3,4- | ESI-MS m/z calc. | ¹H NMR (500 MHz, |
| | difluoro-2-(2-((S)-3- | 574.2015, found 575.3 | DMSO-d₆) δ 10.74 (s, |
| | fluoropyrrolidin-1- | (M + 1)⁺; 573.2 (M − 1)⁻; | 1H), 10.51 (br s, 1H), |
| | yl)ethoxy)phenyl)-4,5- | Retention time: 3.31 | 8.50 (d, J = 5.5 Hz, 1H), |
| | dimethyl-5- | minutes. | 8.35 (s, 1H), 8.06 (d, J = |
| | (trifluoromethyl)tetrahydrofuran- | | 2.6 Hz, 1H), 7.80 (dd, J = |
| | 2-carboxamido)picolinamide | | 5.5, 2.2 Hz, 1H), 7.61 (d, |
| | (Trifluoroacetate salt) | | J = 2.9 Hz, 1H), 7.25 (dd, |
| | | | J = 15.7, 7.1 Hz, 2H), |
| | | | 5.16 (d, J = 10.3 Hz, 1H), |
| | | | 4.43 (d, J = 23.5 Hz, 2H), |
| | | | 4.35-4.26 (m, 1H), 3.50- |
| | | | 3.40 (m, 2H), 3.95 (s, |
| | | | 1H), 3.76 (d, J = 54.8 Hz, |
| | | | 4H), 2.80 (s, 1H), 2.22 (s, |
| | | | 2H), 1.65 (s, 3H), 0.80- |
| | | | 0.60 (m, 3H) ppm. |
| 50 | 4-((2R,3S,4S,5R)-3-(2-(2-(2- | ESI-MS m/z calc. | ¹H NMR (500 MHz, |
| | oxa-5-azabicyclo[4.1.0]heptan- | 584.2058, found 585.3 | Chloroforms-d) δ 8.83 (s, |
| | 5-yl)ethoxy)-3,4- | (M + 1)⁺; 583.2 (M − 1)⁻; | 1H), 8.50 (d, J = 5.6 Hz, |
| | difluorophenyl)-4,5-dimethyl-5- | Retention time: 3.27 | 1H), 8.31 (d, J = 17.7 Hz, |
| | (trifluoromethyl)tetrahydrofuran- | minutes. | 1H), 7.97 (s, 1H), 7.93- |
| | 2-carboxamido)picolinamide | | 7.83 (m, 1H), 7.14 (s, |
| | (Trifluoroacetate salt) | | 1H), 7.09-6.96 (m, 1H), |
| | (Mixture of diastereomers at the | | 6.06 (s, 1H), 4.94 (dd, J = |
| | (2-oxa-5-azabicyclo[4.1.0] | | 10.4, 4.4 Hz, 1H), 4.61 |
| | heptan-5-yl)ethoxy) group.) | | (dd, J = 34.7, 13.6 Hz, |
| | | | 1H), 4.41 (d, J = 40.3 Hz, |
| | | | 1H), 3.96-3.82 (m, 2H), |
| | | | 3.71-3.51 (td, J = 35.1, |
| | | | 32.0, 12.1 Hz, 3H), 3.52- |
| | | | 3.35 (m, 1H), 3.10 (t, J = |
| | | | 7.2 Hz, 1H), 3.03-2.84 |
| | | | (m, 2H), 2.76 (dt, J = |
| | | | 23.3, 11.9 Hz, 1H), 1.72 |
| | | | (s, 3H), 1.34 (d, J = 32.1 |
| | | | Hz, 1H), 1.13 (d, J = 24.9 |
| | | | Hz, 1H), 0.86-0.75 (m, |
| | | | 3H) ppm; H salt not |
| | | | observed. |
| 51 | 4-((2R,3S,4S,5R)-3-(3,4- | ESI-MS m/z calc. | ¹H NMR (500 MHz, |
| | difluoro-2-(2-(2- | 586.22144, found 587.3 | DMSO-d₆) δ 10.75 (s, |
| | methylmorpholino)ethoxy)phenyl)- | (M + 1)⁺; 585.3 (M − 1)⁻; | 1H), 10.11 (br s, 1H), |
| | 4,5-dimethyl-5- | Retention time: 3.25 | 8.50 (d, J = 5.5 Hz, 1H), |
| | (trifluoromethyl)tetrahydrofuran- | minutes. | 8.35 (t, J = 2.5 Hz, 1H), |
| | 2-carboxamido)picolinamide | | 8.06 (d, J = 2.5 Hz, 1H), |
| | (Trifluoroacetate salt) | | 7.80 (dt, J = 5.7, 2.0 Hz, |
| | (Mixture of epimers at the 2- | | 1H), 7.62 (d, J = 2.9 Hz, |
| | methyl stereocenter of the (2- | | 1H), 7.25 (dd, J = 13.4, |
| | methylmorpholino)ethoxy | | 6.9 Hz, 2H), 5.16 (d, J = |
| | group.) | | 10.2 Hz, 1H), 4.48 (s, |
| | | | 2H), 4.30 (ddd, J = 10.7, |
| | | | 7.6, 3.9 Hz, 1H), 4.03 (s, |
| | | | 1H), 3.82 (s, 3H), 3.54 (s, |
| | | | 3H), 3.17 (s, 1H), 2.91 (s, |
| | | | 1H), 2.79 (q, J = 7.5 Hz, |
| | | | 1H), 1.64 (s, 3H), 1.15 (d, |
| | | | J = 6.3 Hz, 3H), 0.80- |
| | | | 0.65 (m, 3H) ppm. |

The following compounds were made using the method described in Example 7, except that the conditions used in step 2 were those described in Example 5 step 2, using different amines:

| Cmpd No. | Compound Name | LC/MS | NMR (shifts in ppm) |
|---|---|---|---|
| 52 | 4-((2R,3S,4S,5R)-3-(3,4- | ESI-MS m/z calc. | ¹H NMR (500 MHz, |
| | difluoro-2-(2-(3- | 572.2058, found 573.3 | Chloroform-d) δ 8.77 (s, |
| | methoxyazetidin-1- | (M + 1)⁺; 571.3 (M − 1)⁻; | 1H), 8.48 (d, J = 5.5 Hz, |
| | yl)ethoxy)phenyl)-4,5- | Retention time: 3.14 | 1H), 8.19 (dd, J = 5.6, 2.3 |
| | dimethyl-5- | minutes | Hz, 1H), 7.96 (d, J = 2.2 |
| | (trifluoromethyl)tetrahydrofuran- | | Hz, 1H), 7.84 (d, J = 5.4 |
| | 2-carboxamido)picolinamide | | Hz, 1H), 7.14-7.09 (m, |
| | | | 1H), 6.98-6.90 (m, 1H), |
| | | | 5.57 (s, 1H), 5.04 (d, J = |
| | | | 11.5 Hz, 1H), 4.42 (dd, J = |
| | | | 11.4, 7.5 Hz, 1H), 4.24 |
| | | | (dt, J = 10.1, 4.4 Hz, 1H), |
| | | | 4.00 (ddd, J = 10.2, 6.6, |
| | | | 3.5 Hz, 1H), 3.86 (q, J = |
| | | | 5.7 Hz, 1H), 3.58 (t, J = |
| | | | 6.7 Hz, 1H), 3.48 (t, J = |
| | | | 6.6 Hz, 1H), 3.12 (s, 3H), |
| | | | 2.95-2.78 (m, 4H), 2.73 |
| | | | (ddd, J = 12.9, 6.4, 3.6 |
| | | | Hz, 1H), 1.76 (s, 3H), |
| | | | 0.80 (dd, J = 7.4, 2.4 Hz, |
| | | | 3H) ppm. |
| 53 | 4-((2R,3S,4S,5R)-3-(2-(2-(2- | ESI-MS m/z calc. | ¹H NMR (500 MHz, |
| | oxa-6-azaspiro[3.3]heptan-6- | 584.2058, found 585.4 | Chloroform-d) δ 8.70 (s, |
| | yl)ethoxy)-3,4-difluorophenyl)- | (M + 1)⁺; 583.3 (M − 1)⁻; | 1H), 8.50 (d, J = 5.5 Hz, |
| | 4,5-dimethyl-5- | Retention time: 2.96 | 1H), 8.14 (dd, J = 5.6, 2.3 |
| | (trifluoromethyl)tetrahydrofuran- | minutes | Hz, 1H), 7.97 (d, J = 2.1 |
| | 2-carboxamido)picolinamide | | Hz, 1H), 7.85 (s, 1H), |
| | | | 7.10 (d, J = 7.9 Hz, 1H), |
| | | | 6.94 (q, J = 8.9 Hz, 1H), |
| | | | 5.58 (s, 1H), 5.04 (d, J = |
| | | | 11.2 Hz, 1H), 4.71-4.64 |
| | | | (m, 4H), 4.22 (dd, J = |
| | | | 11.2, 7.7 Hz, 1H), 4.18- |
| | | | 4.04 (m, 2H), 3.42-3.34 |
| | | | (m, 4H), 2.85 (p, J = 7.7 |
| | | | Hz, 1H), 2.72 (dt, J = |
| | | | 25.9, 8.9 Hz, 2H), 1.70 (s, |
| | | | 3H), 0.81 (d, J = 7.4 Hz, |
| | | | 3H) ppm. |
| 54 | 4-((2R,3S,4S,5R)-3-(2-(2- | ESI-MS m/z calc. | ¹H NMR (500 MHz, |
| | (azetidin-1-yl)ethoxy)-3,4- | 542.19525, found 543.3 | DMSO-d₆) δ 10.62 (s, |
| | difluorophenyl)-4,5-dimethyl-5- | (M + 1)⁺; 541.3 (M − 1)⁻; | 1H), 8.45 (d, J = 5.5 Hz, |
| | (trifluoromethyl)tetrahydrofuran- | Retention time: 3.13 | 1H), 8.27 (d, J = 2.1 Hz, |
| | 2-carboxamido)picolinamide | minutes | 1H), 8.02 (d, J = 2.7 Hz, |
| | | | 1H), 7.82 (dd, J = 5.5, 2.1 |
| | | | Hz, 1H), 7.57 (d, J = 2.8 |
| | | | Hz, 1H), 7.14 (dd, J = |
| | | | 9.5, 6.3 Hz, 2H), 5.10 (d, |
| | | | J = 10.7 Hz, 1H), 4.49- |
| | | | 4.39 (m, 1H), 4.09 (dt, J = |
| | | | 10.4, 4.9 Hz, 1H), 3.92 |
| | | | (s, 1H), 3.25 (s, 1H), |
| | | | 3.20-3.05 (m, 3H), 2.86 |
| | | | (p, J = 7.5 Hz, 1H), 2.75- |
| | | | 2.52 (m, 2H), 1.90-1.80 |
| | | | (m, 2H), 1.68 (s, 3H), |
| | | | 0.72-0.64 (m, 3H) ppm. |

The following compounds were made using the method described in Example 7, except that the conditions used in step 2 were those described in Example 8 step 5, using different amines in excess and no base:

| Cmpd No. | Compound Name | LC/MS | NMR (shifts in ppm) |
|---|---|---|---|
| 55 | 4-((2R,3S,4S,5R)-3-(3,4- | ESI-MS m/z calc. | ¹H NMR (500 MHz, |
| | difluoro-2-(2-((3aR,6aS)- | 598.22144, found 599.4 | Chloroform-d) δ 8.75 (s, |
| | tetrahydro-1H-furo[3,4- | (M + 1)⁺; 597.3 (M − 1)⁻; | 1H), 8.48 (d, J = 5.5 Hz, |
| | c]pyrrol-5(3H)- | Retention time: 3.19 | 1H), 8.16 (s, 1H), 7.97 (s, |
| | yl)ethoxy)phenyl)-4,5- | minutes | 1H), 7.85 (s, 1H), 7.15- |
| | dimethyl-5- | | 7.10 (m 1H), 6.94 (q, J = |
| | (trifluoromethyl)tetrahydrofuran- | | 8.6 Hz, 1H), 5.58 (d, J = |
| | 2-carboxamido)picolinamide | | 4.5 Hz, 1H), 5.06 (s, 1H), |
| | | | 4.38 (q, J = 5.3 Hz, 1H), |
| | | | 4.29 (dd, J = 11.2, 7.9 Hz, |
| | | | 1H), 4.20 (s, 2H), 3.55 |
| | | | (dd, J = 14.6, 5.4 Hz, |
| | | | 3H), 2.86 (p, J = 7.6 Hz, |
| | | | 6H), 1.72 (s, 3H), 1.37- |
| | | | 1.26 (m, 3H), 0.80 (dd, J = |
| | | | 7.5, 2.3 Hz, 3H) ppm. |
| 56 | 4-((2R,3S,4S,5R)-3-(3,4- | ESI-MS m/z calc. | ¹H NMR (500 MHz, |
| | difluoro-2-(2-((R)-3- | 586.22144, found 587.4 | DMSO-d₆) δ 10.69 (s, |
| | methylmorpholino)ethoxy)phenyl)- | (M + 1)⁺; 585.3 (M − 1)⁻; | 1H), 8.49 (d, J = 5.5 Hz, |
| | 4,5-dimethyl-5- | Retention time: 3.23 | 1H), 8.29 (d, J = 2.2 Hz, |
| | (trifluoromethyl)tetrahydrofuran- | minutes | 1H), 8.05 (d, J = 2.7 Hz, |
| | 2-carboxamido)picolinamide | | 1H), 7.84 (dd, J = 5.5, 2.2 |
| | | | Hz, 1H), 7.60 (d, J = 2.8 |
| | | | Hz, 1H), 7.17 (dd, J = |
| | | | 8.7, 4.1 Hz, 2H), 5.13 (d, |
| | | | J = 10.1 Hz, 1H), 4.36- |
| | | | 4.21 (m, 2H), 4.13 (td, J = |
| | | | 10.8, 9.3, 4.6 Hz, 1H), |
| | | | 3.55 (dt, J = 10.7, 2.9 Hz, |
| | | | 2H), 3.44 (ddd, J = 11.4, |
| | | | 9.6, 2.5 Hz, 1H), 3.29 (s, |
| | | | 1H), 3.15-3.00 (m, 2H), |
| | | | 2.83-2.70 (m, 2H), 2.51- |
| | | | 2.41 (m, 1H), 2.25 (ddd, |
| | | | J = 12.6, 9.7, 3.2 Hz, 1H), |
| | | | 1.66 (s, 3H), 0.88 (d, J = |
| | | | 6.3 Hz, 3H), 0.76-0.71 |
| | | | (m, 3H) ppm. |
| 57 | 4-((2R,3S,4S,5R)-3-(2-(2-(1- | ESI-MS m/z calc. | ¹H NMR (500 MHz, |
| | oxa-6-azaspiro[3.3]heptan-6- | 584.2058, found 585.3 | DMSO-d₆) δ 10.69 (s, |
| | yl)ethoxy)-3,4-difluorophenyl)- | (M + 1)⁺; 583.3 (M − 1)⁻; | 1H), 8.48 (d, J = 5.5 Hz, |
| | 4,5-dimethyl-5- | Retention time: 3.03 | 1H), 8.29 (s, 1H), 8.05 (d, |
| | (trifluoromethyl)tetrahydrofuran- | minutes | J = 2.6 Hz, 1H), 7.87- |
| | 2-carboxamido)picolinamide | | 7.82 (m, 1H), 7.60 (s, |
| | | | 1H), 7.19-7.13 (m, 2H), |
| | | | 5.11 (d, J = 10.7 Hz, 1H), |
| | | | 4.42-4.26 (m, 3H), 4.11 |
| | | | (dt, J = 10.2, 5.0 Hz, 1H), |
| | | | 3.98 (dt, J = 10.3, 4.9 Hz, |
| | | | 1H), 3.58-3.51 (m, 2H), |
| | | | 3.09-2.98 (m, 2H), 2.86 |
| | | | (p, J = 7.5 Hz, 1H), 2.69 |
| | | | (ddt, J = 9.8, 5.5, 2.2 Hz, |
| | | | 4H), 1.65 (s, 3H), 0.74- |
| | | | 0.68 (m, 3H) ppm. |

### Example 8

### 4-((2R,3S,4S,5R)-3-(3,4-difluoro-2-(oxetan-3-yloxy)phenyl)-4,5-dimethyl-5-(trifluoromethyl)tetrahydrofuran-2-carboxamido) picolinamide (58)

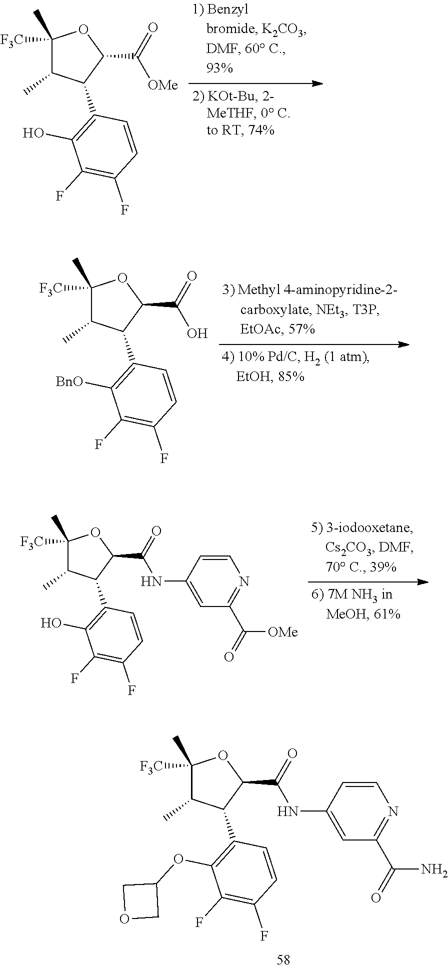 [SMILES: COC(=O)[C@H]1O[C@@](C)(C(F)(F)F)[C@@H](C)[C@H]1c1ccc(F)c(F)c1O ; C[C@H]1[C@@H](c2ccc(F)c(F)c2OCc2ccccc2)[C@H](C(=O)O)O[C@@]1(C)C(F)(F)F ; COC(=O)c1cc(NC(=O)[C@@H]2O[C@@](C)(C(F)(F)F)[C@@H](C)[C@H]2c2ccc(F)c(F)c2O)ccn1 ; C[C@H]1[C@@H](c2ccc(F)c(F)c2OC2COC2)[C@H](C(=O)Nc2ccnc(C(N)=O)c2)O[C@@]1(C)C(F)(F)F]

### Step 1:

Benzyl bromide was added to a stirred mixture of methyl(2S,3S,4S,5R)-3-(3,4-difluoro-2-hydroxyphenyl)-4,5-dimethyl-5-(trifluoromethyl)tetrahydrofuran-2-carboxylate (Product of Example 2,

Step 2, 15 g, 42.34 mmol) and potassium carbonate (7.8 g, 56.44 mmol) in DMF (20 mL). The reaction mixture was stirred at ambient temperature overnight. A further amount of BnBr (2 ml) was added and the reaction was stirred at 60° C. for an additional 3 h. The mixture was partitioned between ethyl acetate (100 ml) and water (150 ml). The aqueous phase was separated and extracted with ethyl acetate (50 mL). The combined organic extracts were washed with water (2×100 ml), brine (1×25 mL), dried over MgSO₄, filtered, and concentrated in vacuo. Purification by flash chromatography (SiO₂, 0 to 100% ethyl acetate in heptane gave methyl(2S,3S,4S,5R)-3-(2-(benzyloxy)-3,4-difluorophenyl)-4,5-dimethyl-5-(trifluoromethyl)tetrahydrofuran-2-carboxylate (17.57 g, 93%) as a colourless oil, which crystalised on standing. ¹H NMR (400 MHZ, Chloroform-d) δ 7.49-7.35 (m, 5H), 7.17 (t, J=7.6 Hz, 1H), 6.82 (td, J=9.3, 7.6 Hz, 1H), 5.12 (dd, J=71.3, 11.2 Hz, 2H), 4.83 (d, J=6.1 Hz, 1H), 4.25 (dd, J=8.6, 6.2 Hz, 1H), 3.53 (s, 3H), 2.73 (p, J=7.8 Hz, 1H), 1.49 (s, 3H), 0.83 (dd, J=7.6, 2.0 Hz, 3H) ppm. ESI-MS m/z calc. 444.136, found 443.1 (M−1); Retention time: 1.09 minutes.

### Step 2:

Potassium tert-butoxide (3.53 g, 31.46 mmol) was added portionwise to a solution of methyl(2S,3S,4S,5R)-3-(2-(benzyloxy)-3,4-difluorophenyl)-4,5-dimethyl-5-(trifluoromethyl)tetrahydrofuran-2-carboxylate (14 g, 31.50 mmol) in 2-MeTHF (10 mL) at 0° C. The reaction mixture was stirred at 0° C. for 5 min, then at ambient temperature for an additional 30 min. The mixture was diluted with MTBE (5 ml) and quenched with 1 M HCl. The aqueous layer was separated and extracted with MTBE (5 ml). The combined organic extracts were dried over MgSO₄, filtered, and concentrated in vacuo to give (2R,3S,4S,5R)-3-(2-(benzyloxy)-3,4-difluorophenyl)-4,5-dimethyl-5-(trifluoromethyl)tetrahydrofuran-2-carboxylic acid (10.5 g, 74%) as an oil. ¹H NMR (500 MHZ, Chloroform-d) δ 7.44-7.28 (m, 5H), 6.98-6.87 (m, 2H), 5.29-5.18 (m, 1H), 5.11 (dd, J=11.3, 1.2 Hz, 1H), 4.87 (d, J=11.0 Hz, 1H), 4.05-3.88 (m, 1H), 2.48 (p, J=7.6 Hz, 1H), 1.42 (d, J=1.2 Hz, 3H), 0.70 (dq, J=7.3, 2.3 Hz, 3H) ppm; acid OH not observed. ESI-MS m/z calc. 430.12036, found 431.2 (M+1)⁺; 429.1 (M−1); Retention time: 0.68 minutes.

### Step 3:

T3P (12.9 mL, 43.38 mmol) was added to a stirred solution of (2R,3S,4S,5R)-3-(2-(benzyloxy)-3,4-difluorophenyl)-4,5-dimethyl-5-(trifluoromethyl)tetrahydrofuran-2-carboxylic acid (2.918 g, 6.780 mmol), methyl 4-aminopyridine-2-carboxylate (1.656 g, 10.88 mmol) and Et₃N (3.0 mL, 21.52 mmol) in EtOAc (27 mL). The mixture was stirred at ambient temperature for 2 h. The reaction mixture was diluted with EtOAc (20 mL) and poured over water (50 mL). The aqueous layer was separated and extracted with EtOAc (2×30 mL). The combined organic extracts were washed with brine (10 mL), dried over MgSO₄, filtered, and concentrated in vacuo. Purification by flash chromatography (80 g SiO₂, 0 to 100% EtOAc in Hexanes) gave methyl 4-((2R,3S,4S,5R)-3-(2-(benzyloxy)-3,4-difluorophenyl)-4,5-dimethyl-5-(trifluoromethyl)tetrahydrofuran-2-carboxamido) picolinate (2.193 g, 57%) as a white foam. ¹H NMR (500 MHZ, Chloroform-d) δ 8.74 (br. s, 1H), 8.65 (d, J=5.5 Hz, 1H), 8.16-8.12 (m, 1H), 7.99-7.95 (m, 1H), 7.34-7.29 (m, 2H), 7.28-7.22 (m, 2H), 7.18 (t, J=7.3 Hz, 1H), 7.12-7.06 (m, 1H), 6.97-6.91 (m, 1H), 5.24 (d, J=11.3 Hz, 1H), 5.08 (d, J=11.3 Hz, 1H), 4.94 (d, J=11.3 Hz, 1H), 4.01 (s, 3H), 3.83-3.77 (m, 1H), 2.53-2.45 (m, 1H), 1.43 (s, 3H), 0.73-0.68 (m, 3H) ppm.

### Step 4:

A solution of methyl 4-((2R,3S,4S,5R)-3-(2-(benzyloxy)-3,4-difluorophenyl)-4,5-dimethyl-5-(trifluoromethyl)tetrahydrofuran-2-carboxamido) picolinate (1.678 g, 2.973 mmol) in EtOH (35 mL) was added to Pd/C (87 mg, 0.8175 mmol). The mixture was purged with nitrogen for 5 min, and then sparged with hydrogen for 5 min. The reaction mixture was stirred under a hydrogen atmosphere (via balloon) at ambient temperature for 16 h. The reaction mixture was filtered through a pad of celite, and washed with EtOH (3×100 mL). The filtrate was concentrated in vacuo to give an off white solid (1.35 g). The solid was stirred in an ice cold 2:1 DCM: heptane solution (30 mL). The suspension was filtered to give methyl 4-((2R,3S,4S,5R)-3-(3,4-difluoro-2-hydroxyphenyl)-4,5-dimethyl-5-(trifluoromethyl)tetrahydrofuran-2-carboxamido) picolinate (1.205 g, 85%) as a white solid. ¹H NMR (500 MHZ, DMSO-d₆) δ 10.79 (s, 1H), 10.47 (s, 1H), 8.57 (d, J=5.5 Hz, 1H), 8.35 (d, J=2.1 Hz, 1H), 7.84 (dd, J=5.5, 2.1 Hz, 1H), 7.05-7.00 (m, 1H), 6.88-6.81 (m, 1H), 5.11 (d, J=10.2 Hz, 1H), 4.26 (dd, J=10.2, 7.6 Hz, 1H), 3.87 (s, 3H), 2.87-2.80 (m, 1H), 1.60 (s, 3H), 0.71 (d, J=6.2 Hz, 3H) ppm. ESI-MS m/z calc. 474.1214, found 474.9 (M+1)⁺; 473.0 (M−1); Retention time: 0.98 minutes.

### Step 5:

3-Iodooxetane (133 mg, 0.7229 mmol) was added to a solution of methyl 4-((2R,3S,4S,5R)-3-(3,4-difluoro-2-hydroxyphenyl)-4,5-dimethyl-5-(trifluoromethyl)tetrahydrofuran-2-carboxamido) picolinate (80 mg, 0.1447 mmol) and cesium carbonate (100 mg, 0.3069 mmol) in DMF (1 mL). The mixture was heated to 70° C. for 12 h. The reaction mixture was cooled to ambient temperature and partitioned between MTBE (20 ml) and water (20 ml). The aqueous layer was separated and extracted with MTBE (10 mL). The combined organic extracts were washed with brine (1×10 mL), dried over MgSO₄, filtered, and concentrated in vacuo to give methyl 4-((2R,3S,4S,5R)-3-(3,4-difluoro-2-(oxetan-3-yloxy)phenyl)-4,5-dimethyl-5-(trifluoromethyl)tetrahydrofuran-2-carboxamido) picolinate (100 mg, 39%), which was used in the next step without further purification. ESI-MS m/z calc. 530.14764, found 531.3 (M+1)⁺; 529.2 (M−1); Retention time: 0.93 minutes.

### Step 6:

Methanolic ammonia (322 μL of 7 M, 2.254 mmol) was added to a stirred solution of methyl 4-((2R,3S,4S,5R)-3-(3,4-difluoro-2-(oxetan-3-yloxy)phenyl)-4,5-dimethyl-5-(trifluoromethyl)tetrahydrofuran-2-carboxamido) picolinate (90 mg, 0.051 mmol) in methanol (2 mL). The reaction mixture was stirred overnight at ambient temperature. The mixture was concentrated in vacuo. Purification by reverse phase preparative HPLC gave 4-((2R,3S,4S,5R)-3-(3,4-difluoro-2-(oxetan-3-yloxy)phenyl)-4,5-dimethyl-5-(trifluoromethyl)tetrahydrofuran-2-carboxamido) picolinamide (58, 16 mg, 61%). ¹H NMR (500 MHZ, DMSO-d₆) § 10.71 (s, 1H), 8.50 (d, J=5.5 Hz, 1H), 8.28 (d, J=2.2 Hz, 1H), 8.06 (d, J=2.7 Hz, 1H), 7.84 (dd, J=5.5, 2.2 Hz, 1H), 7.61 (d, J=2.9 Hz, 1H), 7.21-7.13 (m, 2H), 5.30 (dddd, J=10.9, 6.0, 4.7, 3.3 Hz, 1H), 5.13 (d, J=10.3 Hz, 1H), 4.90-4.83 (m, 2H), 4.70 (ddd, J=11.1, 7.5, 4.9 Hz, 2H), 4.28 (dd, J=10.3, 7.5 Hz, 1H), 2.80 (p, J=7.5 Hz, 1H), 1.63 (s, 3H), 0.77-0.70 (m, 3H) ppm. ESI-MS m/z calc. 515.14795, found 516.3 (M+1)⁺; 514.2 (M−1); Retention time: 3.05 minutes.

The following compounds were made using the method described in Example 8, except that different alkylating agents were used in step 5:

| Cmpd No. | Compound Name | LC/MS | NMR (shifts in ppm) |
|---|---|---|---|
| 59 | rel-4-((2R*,3S*,4S*,5R*)-3- | ESI-MS m/z calc. | ¹H NMR (500 MHz, |
| | (3,4-difluoro-2-((oxetan-2- | 529.16364, found 530.3 | DMSO-d₆) δ 10.66 (s, |
| | yl)methoxy)phenyl)-4,5- | (M + 1)⁺; 528.2 (M − 1)⁻; | 1H), 8.49 (d, J = 5.5 Hz, |
| | dimethyl-5- | Retention time: 3.15 | 1H), 8.30 (d, J = 2.2 Hz, |
| | (trifluoromethyl)tetrahydrofuran- | minutes | 1H), 8.06 (d, J = 2.7 Hz, |
| | 2-carboxamido)picolinamide | | 1H), 7.84 (dd, J = 5.5, 2.2 |
| | | | Hz, 1H), 7.61 (d, J = 2.9 |
| | | | Hz, 1H), 7.23-7.17 (m, |
| | | | 2H), 5.12 (d, J = 10.5 Hz, |
| | | | 1H), 5.01 (dddd, J = 8.3, |
| | | | 6.7, 5.0, 3.7 Hz, 1H), 4.55- |
| | | | 4.43 (m, 2H), 4.43- |
| | | | 4.37 (m, 2H), 4.27-4.21 |
| | | | (m, 2H), 2.83 (p, J = 7.5 |
| | | | Hz, 1H), 2.69 (dtd, J = |
| | | | 11.1, 8.4, 6.3 Hz, 1H), |
| | | | 1.60 (s, 3H), 0.76-0.70 |
| | | | (m, 3H) ppm. |
| 60 | rel-4-((2R*,3S*,4S*,5R*)-3- | ESI-MS m/z calc. | ¹H NMR (500 MHz, |
| | (3,4-difluoro-2-((-oxetan-2- | 529.16364, found 530.3 | DMSO-d₆) δ 10.64 (s, |
| | yl)methoxy)phenyl)-4,5- | (M + 1)⁺; 528.2 (M − 1)⁻; | 1H), 8.49 (d, J = 5.5 Hz, |
| | dimethyl-5- | Retention time: 3.18 | 1H), 8.30 (d, J = 2.1 Hz, |
| | (trifluoromethyl)tetrahydrofuran- | minutes | 1H), 8.05 (d, J = 2.7 Hz, |
| | 2-carboxamido)picolinamide | | 1H), 7.83 (dd, J = 5.5, 2.2 |
| | | | Hz, 1H), 7.61 (d, J = 2.9 |
| | | | Hz, 1H), 7.24-7.17 (m, |
| | | | 2H), 5.12 (d, J = 10.4 Hz, |
| | | | 1H), 5.01-4.92 (m, 1H), |
| | | | 4.52-4.41 (m, 2H), 4.39- |
| | | | 4.29 (m, 2H), 4.16- |
| | | | 4.10 (m, 1H), 2.84 (p, J = |
| | | | 7.4 Hz, 1H), 2.70-2.59 |
| | | | (m, 1H), 2.59-2.51 (m, |
| | | | 1H), 1.60 (s, 3H), 0.78- |
| | | | 0.72 (m, 3H) ppm. |
| 61 | 4-((2R,3S,4S,5R)-3-(3,4- | ESI-MS m/z calc. | ¹H NMR (500 MHz, |
| | difluoro-2-((1-methyl-1H- | 553.17485, found 553.9 | DMSO-d₆) δ 10.64 (s, |
| | imidazol-2- | (M + 1)⁺; 552.0 (M − 1)⁻; | 1H), 8.49 (d, J = 5.5 Hz, |
| | yl)methoxy)phenyl)-4,5- | Retention time: 2.97 | 1H), 8.27 (d, J = 2.1 Hz, |
| | dimethyl-5- | minutes | 1H), 8.05 (d, J = 2.8 Hz, |
| | (trifluoromethyl)tetrahydrofuran- | | 1H), 7.82 (dd, J = 5.5, 2.1 |
| | 2-carboxamido)picolinamide | | Hz, 1H), 7.60 (d, J = 2.8 |
| | | | Hz, 1H), 7.25-7.14 (m, |
| | | | 3H), 6.81 (d, J = 0.8 Hz, |
| | | | 1H), 5.25 (d, J = 12.3 Hz, |
| | | | 1H), 5.14 (d, J = 12.3 Hz, |
| | | | 1H), 5.06 (d, J = 10.5 Hz, |
| | | | 1H), 4.08 (dd, J = 10.5, |
| | | | 7.4 Hz, 1H), 3.73 (s, 3H), |
| | | | 2.48-2.42 (m, 1H), 1.47 |
| | | | (s, 3H), 0.66 (d, J = 6.4 |
| | | | Hz, 3H) ppm. |
| 62 | 4-((2R,3S,4S,5R)-3-(3,4- | ESI-MS m/z calc. | ¹H NMR (500 MHz, |
| | difluoro-2-(3- | 586.22144, found 586.8 | DMSO-d₆) δ 10.70 (s, |
| | morpholinopropoxy)phenyl)- | (M + 1)⁺; 585.1 (M − 1)⁻; | 1H), 8.49 (d, J = 5.5 Hz, |
| | 4,5-dimethyl-5- | Retention time: 3.46 | 1H), 8.28 (d, J = 2.2 Hz, |
| | (trifluoromethyl)tetrahydrofuran- | minutes | 1H), 8.05 (d, J = 2.8 Hz, |
| | 2-carboxamido)picolinamide | | 1H), 7.82 (dd, J = 5.5, 2.2 |
| | | | Hz, 1H), 7.61 (d, J = 2.8 |
| | | | Hz, 1H), 7.20-7.13 (m, |
| | | | 2H), 5.11 (d, J = 10.3 Hz, |
| | | | 1H), 4.27 (dd, J = 10.3, |
| | | | 7.7 Hz, 1H), 4.24-4.19 |
| | | | (m, 1H), 4.13-4.07 (m, |
| | | | 1H), 3.51 (t, J = 4.6 Hz, |
| | | | 4H), 2.77-2.70 (m, 1H), |
| | | | 2.44-2.40 (m, 2H), 2.35- |
| | | | 2.26 (m, 4H), 1.92- |
| | | | 1.86 (m, 2H), 1.62 (s, |
| | | | 3H), 0.72 (d, J = 2.5 Hz, |
| | | | 3H) ppm. |
| 63 | 4-((2R,3S,4S,5R)-3-(3,4- | ESI-MS m/z calc. | ¹H NMR (500 MHz, |
| | difluoro-2-((3-methyloxetan-3- | 543.17926, found 544.3 | DMSO-d₆) δ 10.74 (s, |
| | yl)methoxy)phenyl)-4,5- | (M + 1)⁺; 542.2 (M − 1)⁻; | 1H), 8.49 (d, J = 5.5 Hz, |
| | dimethyl-5- | Retention time: 3.22 | 1H), 8.28 (d, J = 2.1 Hz, |
| | (trifluoromethyl)tetrahydrofuran- | minutes. | 1H), 8.06 (d, J = 2.7 Hz, |
| | 2-carboxamido)picolinamide | | 1H), 7.82 (dd, J = 5.5, 2.2 |
| | | | Hz, 1H), 7.61 (d, J = 2.8 |
| | | | Hz, 1H), 7.23-7.16 (m, |
| | | | 2H), 5.13 (d, J = 10.4 Hz, |
| | | | 1H), 4.54 (dd, J = 5.9, 4.5 |
| | | | Hz, 2H), 4.37-4.29 (m, |
| | | | 4H), 4.14 (dd, J = 9.4, 1.6 |
| | | | Hz, 1H), 2.76 (p, J = 7.4 |
| | | | Hz, 1H), 1.59 (s, 3H), |
| | | | 1.40 (s, 3H), 0.77-0.71 |
| | | | (m, 3H) ppm. |
| 64 | 4-((2R,3S,4S,5R)-3-(3,4- | ESI-MS m/z calc. | ¹H NMR (500 MHz, |
| | difluoro-2-((3-fluorooxetan-3- | 547.1542, found 548.2 | DMSO-d₆) δ 10.72 (s, |
| | yl)methoxy)phenyl)-4,5- | (M + 1)⁺; 546.2 (M − 1)⁻; | 1H), 8.50 (d, J = 5.6 Hz, |
| | dimethyl-5- | Retention time: 3.15 | 1H), 8.29 (d, J = 2.1 Hz, |
| | (trifluoromethyl)tetrahydrofuran- | minutes | 1H), 8.06 (d, J = 2.7 Hz, |
| | 2-carboxamido)picolinamide | | 1H), 7.83 (dd, J = 5.5, 2.2 |
| | | | Hz, 1H), 7.61 (d, J = 2.8 |
| | | | Hz, 1H), 7.27-7.16 (m, |
| | | | 2H), 5.13 (d, J = 10.6 Hz, |
| | | | 1H), 4.80-4.62 (m, 5H), |
| | | | 4.55 (dd, J = 21.9, 11.4 |
| | | | Hz, 1H), 4.26 (dd, J = |
| | | | 10.5, 7.3 Hz, 1H), 2.73 |
| | | | (p, J = 7.4 Hz, 1H), 1.59 |
| | | | (s, 3H), 0.72 (dd, J = 7.3, |
| | | | 2.4 Hz, 3H) ppm. |
| 65 | 4-((2R,3S,4S,5R)-3-(3,4- | ESI-MS m/z calc. | ¹H NMR (500 MHz, |
| | difluoro-2-((tetrahydro-2H- | 543.17926, found 544.2 | DMSO-d₆) δ 10.73 (s, |
| | pyran-4-yl)oxy)phenyl)-4,5- | (M + 1)⁺; 542.2 (M − 1)⁻; | 1H), 8.50 (d, J = 5.5 Hz, |
| | dimethyl-5- | Retention time: 3.24 | 1H), 8.27 (d, J = 2.2 Hz, |
| | (trifluoromethyl)tetrahydrofuran- | minutes. | 1H), 8.06 (d, J = 2.7 Hz, |
| | 2-carboxamido)picolinamide | | 1H), 7.81 (dd, J = 5.5, 2.2 |
| | | | Hz, 1H), 7.61 (d, J = 2.8 |
| | | | Hz, 1H), 7.23-7.15 (m, |
| | | | 2H), 5.10 (d, J = 10.5 Hz, |
| | | | 1H), 4.46 (tt, J = 9.0, 4.2 |
| | | | Hz, 1H), 4.32 (dd, J = |
| | | | 10.5, 7.6 Hz, 1H), 3.89 |
| | | | (dt, J = 11.7, 4.3 Hz, 1H), |
| | | | 3.78 (dt, J = 11.9, 4.3 Hz, |
| | | | 1H), 3.42 (ddd, J = 11.6, |
| | | | 10.1, 2.8 Hz, 1H), 3.38- |
| | | | 3.27 (m, 1H), 2.76 (p, J = |
| | | | 7.5 Hz, 1H), 2.08-2.00 |
| | | | (m, 1H), 1.85 (ddd, J = |
| | | | 12.9, 4.5, 2.3 Hz, 1H), |
| | | | 1.77-1.66 (m, 1H), 1.63- |
| | | | 1.54 (m, 4H), 0.73 (d, J = |
| | | | 6.9 Hz, 3H) ppm. |

The following compounds were made using the method described in Example 8, except that different alkylating agents were used in step 5. In step 6, a methylamine solution (33 wt. % in absolute ethanol) was used in place of methanolic ammonia:

| Cmpd No. | Compound Name | LC/MS | NMR (shifts in ppm) |
|---|---|---|---|
| 66 | 4-((2R,3S,4S,5R)-3-(3,4- | ESI-MS m/z calc. | ¹H NMR (500 MHz, |
| | difluoro-2-(oxetan-3- | 543.17926, found 544.2 | DMSO-d₆) δ 10.74 (s, |
| | ylmethoxy)phenyl)-4,5- | (M + 1)⁺; 542.2 (M − 1)⁻; | 1H), 8.72 (q, J = 4.8 Hz, |
| | dimethyl-5- | Retention time: 3.16 | 1H), 8.50 (d, J = 5.5 Hz, |
| | (trifluoromethyl)tetrahydrofuran- | minutes. | 1H), 8.26 (d, J = 2.1 Hz, |
| | 2-carboxamido)-N- | | 1H), 7.86 (dd, J = 5.5, 2.2 |
| | methylpicolinamide | | Hz, 1H), 7.19 (dd, J = |
| | | | 8.4, 5.0 Hz, 2H), 5.12 (d, |
| | | | J = 10.4 Hz, 1H), 4.72 |
| | | | (dd, J = 7.9, 6.1 Hz, 2H), |
| | | | 4.51-4.42 (m, 3H), 4.29 |
| | | | (ddd, J = 13.4, 8.7, 6.4 |
| | | | Hz, 2H), 3.40 (dtd, J = |
| | | | 12.0, 6.0, 1.8 Hz, 1H), |
| | | | 2.81 (d, J = 4.8 Hz, 3H), |
| | | | 2.75 (q, J = 7.5 Hz, 1H), |
| | | | 1.60 (s, 3H), 0.77-0.69 |
| | | | (m, 3H) ppm. |

The following compounds were made using the method described in Example 8, except that different alkylating agents were used in step 5. At the end of the synthesis, the enantiomers were further separated by chiral SFC using a Chiralpak AD-H column, 5 mm particle size, 25 cm×10 mm from Daicel Corporation (Mobile phase: 25% methanol: isopropanol (in a 1:1 ratio, supplemented with 20 mM NH₃); System pressure: 80 bar) on a Minigram SFC instrument from Berger Instruments:

| Cmpd No. | Compound Name | LC/MS | NMR (shifts in ppm) |
|---|---|---|---|
| 67 | rel-4-((2R*,3S*,4S*,5R*)-3- | ESI-MS m/z calc. | ¹H NMR (500 MHz, |
| | (3,4-difluoro-2- | 543.17926, found 544.2 | Chloroform-d) δ 8.88 (s, |
| | ((tetrahydrofuran-3- | (M + 1)⁺; 542.2 (M − 1)⁻; | 1H), 8.49 (d, J = 5.5 Hz, |
| | yl)methoxy)phenyl)-4,5- | Retention time: 3.22 | 1H), 8.19 (dd, J = 5.6, 2.2 |
| | dimethyl-5- | minutes | Hz, 1H), 8.07 (d, J = 2.2 |
| | (trifluoromethyl)tetrahydrofuran- | | Hz, 1H), 7.94 (s, 1H), |
| | 2-carboxamido)picolinamide | | 7.12 (ddd, J = 8.2, 5.4, |
| | (First eluting isomer by SFC on | | 2.0 Hz, 1H), 6.99-6.86 |
| | Chiralpak AD-H column, rt = | | (m, 1H), 5.74 (d, J = 3.7 |
| | 3.12 min) | | Hz, 1H), 5.05 (d, J = 10.9 |
| | | | Hz, 1H), 4.24 (ddd, J = |
| | | | 8.8, 6.5, 1.9 Hz, 1H), 4.12 |
| | | | (dd, J = 10.9, 8.0 Hz, |
| | | | 1H), 4.03 (ddd, J = 9.3, |
| | | | 7.8, 1.5 Hz, 1H), 3.93- |
| | | | 3.67 (m, 4H), 2.81-2.68 |
| | | | (m, 2H), 2.09 (dddd, J = |
| | | | 12.7, 8.6, 7.6, 5.3 Hz, |
| | | | 1H), 1.76-1.66 (m, 4H), |
| | | | 0.82 (dq, J = 7.4, 2.4 Hz, |
| | | | 3H) ppm. |
| 68 | rel-4-((2R*,3S*,4S*,5R*)-3- | ESI-MS m/z calc. | ¹H NMR (500 MHz, |
| | (3,4-difluoro-2- | 543.17926, found 544.2 | DMSO-d₆) δ 10.73 (s, |
| | ((tetrahydrofuran-3- | (M + 1)⁺; 542.2 (M − 1)⁻; | 1H), 8.50 (d, J = 5.5 Hz, |
| | yl)methoxy)phenyl)-4,5- | Retention time: 3.19 | 1H), 8.29 (d, J = 2.1 Hz, |
| | dimethyl-5- | minutes | 1H), 8.06 (d, J = 2.8 Hz, |
| | (trifluoromethyl)tetrahydrofuran- | | 1H), 7.83 (dd, J = 5.5, 2.2 |
| | 2-carboxamido)picolinamide | | Hz, 1H), 7.61 (d, J = 2.8 |
| | (Second eluting isomer by SFC | | Hz, 1H), 7.22-7.15 (m, |
| | on Chiralpak AD-H column, rt = | | 2H), 5.12 (d, J = 10.3 Hz, |
| | 4.40 min) | | 1H), 4.28 (dd, J = 10.4, |
| | | | 7.6 Hz, 1H), 4.14 (ddd, J = |
| | | | 9.3, 7.5, 1.7 Hz, 1H), |
| | | | 4.05-3.97 (m, 1H), 3.84- |
| | | | 3.71 (m, 2H), 3.67- |
| | | | 3.57 (m, 2H), 2.79-2.63 |
| | | | (m, 2H), 2.08-1.97 (m, |
| | | | 1H), 1.71-1.63 (m, 1H), |
| | | | 1.62 (s, 3H), 0.76-0.70 |
| | | | (m, 3H) ppm. |

The following compounds were made using the method described in Example 8, except that the conditions used in step 5 were those described in Example 5 step 2 using different alkylating agents. The reaction was carried out at ambient temperature:

| Cmpd No. | Compound Name | LC/MS | NMR (shifts in ppm) |
|---|---|---|---|
| 69 | 4-((2R,3S,4S,5R)-3-(2- | ESI-MS m/z calc. | ¹H NMR (500 MHz, |
| | (cyanomethoxy)-3,4- | 498.13266, found 499.2 | DMSO-d₆) δ 10.66 (s, |
| | difluorophenyl)-4,5-dimethyl-5- | (M + 1)⁺; 497.1 (M − 1)⁻; | 1H), 8.49 (d, J = 5.5 Hz, |
| | (trifluoromethyl)tetrahydrofuran- | Retention time: 3.06 | 1H), 8.29 (d, J = 2.1 Hz, |
| | 2-carboxamido)picolinamide | minutes | 1H), 8.05 (s, 1H), 7.84 |
| | | | (dd, J = 5.5, 2.2 Hz, 1H), |
| | | | 7.60 (d, J = 2.8 Hz, 1H), |
| | | | 7.36-7.21 (m, 2H), 5.26 |
| | | | (s, 2H), 5.14 (d, J = 10.2 |
| | | | Hz, 1H), 4.28 (dd, J = |
| | | | 10.2, 7.5 Hz, 1H), 2.83 |
| | | | (p, J = 7.5 Hz, 1H), 1.64 |
| | | | (s, 3H), 0.75 (d, J = 7.3 |
| | | | Hz, 3H) ppm. |
| 70 | 4-((2R,3S,4S,5R)-3-(3,4- | ESI-MS m/z calc. | ¹H NMR (500 MHz, |
| | difluoro-2-(oxetan-3- | 529.1636, found 530.1 | Chloroform-d) δ 8.67 (s, |
| | ylmethoxy)phenyl)-4,5- | (M + 1)⁺; 528.0 (M − 1)⁻; | 1H), 8.45 (d, J = 5.5 Hz, |
| | dimethyl-5- | Retention time: 2.36 | 1H), 8.10 (q, J = 2.6 Hz, |
| | (trifluoromethyl)tetrahydrofuran- | minutes | 1H), 7.97 (d, J = 2.3 Hz, |
| | 2-carboxamido)picolinamide | | 1H), 7.82 (s, 1H), 7.12- |
| | | | 7.08 (m, 1H), 6.96-6.90 |
| | | | (m, 1H), 5.60 (s, 1H), |
| | | | 5.00 (d, J = 11.0 Hz, 1H), |
| | | | 4.81 (ddd, J = 21.3, 7.8, |
| | | | 6.2 Hz, 2H), 4.58 (t, J = |
| | | | 6.2 Hz, 1H), 4.51 (qd, J = |
| | | | 6.2, 2.9 Hz, 2H), 4.28 |
| | | | (qd, J = 5.3, 1.8 Hz, 1H), |
| | | | 4.17 (dd, J = 11.2, 8.0 Hz, |
| | | | 1H), 3.37-3.31 (m, 1H), |
| | | | 2.73 (t, J = 7.8 Hz, 1H), |
| | | | 1.67 (s, 3H), 0.81-0.78 |
| | | | (m. 3H) ppm. |

The following compound was made using the method described in Example 8, except that the order in which steps 5 and 6 was carried out was reversed and different alkylating agents were used in step 5:

| | | | |
|---|---|---|---|
| Cmpd No. | Compound Name | LC/MS | NMR (shifts in ppm) |

| | | |
|---|---|---|
| 71 | 4-((2R,3S,4S,5R)-3-(3,4- | ESI-MS m/z calc. |
| | difluoro-2-(3-hydroxy-2,2- | 545.19491, found 546.2 |
| | dimethylpropoxy)phenyl)-4,5- | (M + 1)⁺; 544.2 (M − 1)⁻; |
| | dimethyl-5- | Retention time: 3.26 |
| | (trifluoromethyl)tetrahydrofuran- | minutes |
| | 2-carboxamido)picolinamide | |

The following compounds were made using the method described in Example 8, except that the order in which steps 5 and 6 was carried out was reversed. The conditions used in step 5 were those described in Example 5 step 2, using different alkylating agents and running the reaction at 50° C.:

| Cmpd No. | Compound Name | LC/MS | NMR (shifts in ppm) |
|---|---|---|---|
| 72 | 4-((2R,3S,4S,5R)-3-(3,4- | ESI-MS m/z calc. | |
| | difluoro-2-(3- | 517.16364, found 518.2 | |
| | hydroxypropoxy)phenyl)-4,5- | (M + 1)⁺; 516.2 (M − 1)⁻; | |
| | dimethyl-5- | Retention time: 2.92 | |
| | (trifluoromethyl)tetrahydrofuran- | minutes | |
| | 2-carboxamido)picolinamide | | |
| 73 | 4-((2R,3S,4S,5R)-3-(3,4- | ESI-MS m/z calc. | ¹H NMR (500 MHz, |
| | difluoro-2-((1-methyl-1H- | 553.17487, found 554.7 | Chloroform-d) δ 8.92 (s, |
| | pyrazol-4-yl)methoxy)phenyl)- | (M + 1)⁺; 552.6 (M − 1)⁻; | 1H), 8.52 (d, J = 5.7 Hz, |
| | 4,5-dimethyl-5- | Retention time: 3.09 | 1H), 8.31 (s, 1H), 8.25 (d, |
| | (trifluoromethyl)tetrahydrofuran- | minutes | J = 5.6 Hz, 1H), 8.20- |
| | 2-carboxamido)picolinamide | | 8.11 (m, 1H), 7.49 (s, |
| | | | 1H), 7.37 (s, 1H), 7.08 |
| | | | (dd, J = 9.2, 5.2 Hz, 1H), |
| | | | 6.96 (q, J = 8.7 Hz, 1H), |
| | | | 5.84 (s, 1H), 5.18 (d, J = |
| | | | 11.9 Hz, 1H), 5.07 (d, J = |
| | | | 11.9 Hz, 1H), 4.99 (d, J = |
| | | | 11.0 Hz, 1H), 3.98 (dd, J = |
| | | | 11.0, 8.0 Hz, 1H), 3.82 |
| | | | (s, 3H), 2.61 (p, J = 7.6 |
| | | | Hz, 1H), 1.59 (s, 3H), |
| | | | 0.82-0.66 (m, 3H) ppm. |

The following compounds were made using the method described in Example 8, except that step 5 was carried out at ambient temperature with DMSO as the solvent and using different alkylating agents:

| Cmpd No. | Compound Name | LC/MS | NMR (shifts in ppm) |
|---|---|---|---|
| 74 | 4-((2R,3S,4S,5R)-3-(2-(3- | ESI-MS m/z calc. | ¹H NMR (500 MHz, |
| | cyano-3-methylbutoxy)-3,4- | 554.19525, found 555.5 | DMSO-d₆) δ 10.71 (s, |
| | difluorophenyl)-4,5-dimethyl-5- | (M + 1)⁺; 553.5 (M − 1)⁻; | 1H), 8.49 (d, 1H), 8.28 |
| | (trifluoromethyl)tetrahydrofuran- | Retention time: 3.41 | (d, 1H), 8.05 (m, 1H), |
| | 2-carboxamido)picolinamide | minutes | 7.83-7.82 (m, 1H), 7.60 |
| | | | (m, 1H), 7.22-7.19 (m, |
| | | | 2H), 5.12-5.10 (d, 1H), |
| | | | 4.32-428 (m, 2H), 4.25- |
| | | | 4.23 (m, 1H), 2.82-2.79 |
| | | | (m, 1H), 2.10-2.07 (m, |
| | | | 2H), 1.63 (s, 3H), 1.39 (d, |
| | | | 6H), 0.73 (m, 3H) ppm. |
| 75 | 4-((2R,3S,4S,5R)-3-(3,4- | ESI-MS m/z calc. | |
| | difluoro-2-((2- | 567.14044, found 568.3 | |
| | (trifluoromethyl)allyl)oxy)phenyl)- | (M + 1)⁺; 566.3 (M − 1)⁻; | |
| | 4,5-dimethyl-5- | Retention time: 3.62 | |
| | (trifluoromethyl)tetrahydrofuran- | minutes | |
| | 2-carboxamido)picolinamide | | |

The following compounds were made using the method described in Example 8, except that step 5 was carried out at ambient temperature in the presence of an excess of sodium iodine with DMSO as the solvent and using different alkylating agents:

| Cmpd No. | Compound Name | LC/MS | NMR (shifts in ppm) |
|---|---|---|---|
| 76 | 4-((2R,3S,4S,5R)-3-(2-(3- | ESI-MS m/z calc. | |
| | cyanopropoxy)-3,4- | 526.16394, found 527.3 | |
| | difluorophenyl)-4,5-dimethyl-5- | (M + 1)⁺; 525.3 (M − 1)⁻; | |
| | (trifluoromethyl)tetrahydrofuran- | Retention time: 3.18 | |
| | 2-carboxamido)picolinamide | minutes | |
| 77 | 4-((2R,3S,4S,5R)-3-(2-(2-(1- | ESI-MS m/z calc. | ¹H NMR (500 MHz, |
| | cyanocyclopropyl)ethoxy)-3,4- | 552.1796, found 553.3 | Methanol-d₄) δ 8.50 (d, |
| | difluorophenyl)-4,5-dimethyl-5- | (M + 1)⁺; 551.3 (M − 1)⁻; | J = 5.5 Hz, 1H), 8.29 (d, |
| | (trifluoromethyl)tetrahydrofuran- | Retention time: 3.32 | J = 2.1 Hz, 1H), 7.90 (dd, |
| | 2-carboxamido)picolinamide | minutes | J = 5.5, 2.2 Hz, 1H), 7.19 |
| | | | (ddd, J = 8.2, 5.6, 2.1 Hz, |
| | | | 1H), 7.04 (td, J = 9.4, 7.6 |
| | | | Hz, 1H), 5.09 (d, J = 10.1 |
| | | | Hz, 1H), 4.47 (dd, J = |
| | | | 10.1, 8.5 Hz, 1H), 4.45- |
| | | | 4.35 (m, 1H), 4.28 (dt, J = |
| | | | 10.1, 6.0 Hz, 1H), 2.92 |
| | | | (p, J = 7.7 Hz, 1H), 2.08- |
| | | | 1.96 (m, 2H), 1.70 (s, |
| | | | 3H), 1.34-1.25 (m, 2H), |
| | | | 1.07-0.98 (m, 2H), 0.85 |
| | | | (dq, J = 7.4, 2.4 Hz, 3H) |
| | | | ppm; amides NH and |
| | | | NH₂ not observed. |

The following compounds were made using the method described in Example 8, except that the order in which steps 5 and 6 was carried out was reversed. In step 5, K₂CO₃ was used as the base together with different alkylating agents:

| Cmpd No. | Compound Name | LC/MS | NMR (shifts in ppm) |
|---|---|---|---|
| 78 | 4-((2R,3S,4S,5R)-3-(2-(2,3- | ESI-MS m/z calc. | ¹H NMR (500 MHz, |
| | dimethoxypropoxy)-3,4- | 561.1898, found 562.5 | Methanol-d₄) δ 8.51 (d, |
| | difluorophenyl)-4,5-dimethyl-5- | (M + 1)⁺; 561.5 (M − 1)⁻; | 1H), 8.29 (d, 1H), 7.92 |
| | (trifluoromethyl)tetrahydrofuran- | Retention time: 3.29 | (dd, 1H), 7.19-7.14 (m, |
| | 2-carboxamido)picolinamide | minutes. | 1H), 7.06-6.98 (m, 1H), |
| | (Mixture of epimers at the | | 5.12-5.10 (d, 1H), 4.51- |
| | 2,3-dimethoxypropoxy group.) | | 4.49 (m, 1H), 4.32 (m, |
| | | | 1H), 4.21 (m, 1H), 3.65- |
| | | | 3.60 (m, 3H), 3.40 (m, |
| | | | 6H), 2.82 (m, 1H), 1.71 |
| | | | (s, 3H), 0.83-0.81 (m, |
| | | | 3H) ppm; amides NH and |
| | | | NH₂ not observed. |
| 79 | 4-((2R,3S,4S,5R)-3-(3,4- | ESI-MS m/z calc. | ¹H NMR (500 MHz, |
| | difluoro-2-(2- | 515.14795, found 516.6 | Methanol-d₄) δ 8.48 (d, J = |
| | oxopropoxy)phenyl)-4,5- | (M + 1)⁺; 514.4 (M − 1)⁻; | 5.5 Hz, 1H), 8.27 (d, J = |
| | dimethyl-5- | Retention time: 0.88 | 2.1 Hz, 1H), 7.88 (ddd, |
| | (trifluoromethyl)tetrahydrofuran- | minutes | J = 10.6, 5.5, 2.2 Hz, 1H), |
| | 2-carboxamido)picolinamide | | 7.16 (ddd, J = 8.1, 5.5, |
| | | | 2.2 Hz, 1H), 7.04-6.95 |
| | | | (m, 1H), 5.11-5.06 (m, |
| | | | 1H), 5.03 (dd, J = 17.5, |
| | | | 1.8 Hz, 1H), 4.87 (d, J = |
| | | | 1.8 Hz, 1H), 4.63-4.45 |
| | | | (m, 1H), 2.91 (p, J = 7.4 |
| | | | Hz, 1H), 2.14 (s, 3H), |
| | | | 1.70 (dd, J = 10.4, 2.0 Hz, |
| | | | 3H), 0.83 (dt, J = 7.3, 2.4 |
| | | | Hz, 3H) ppm; amides NH |
| | | | and NH₂ not observed. |

The following compound was made using the method described in Example 8, except that the order in which steps 5 and 6 was carried out was reversed. In step 5, K₂CO₃ was used as the base together with different alkylating agents and the reaction was carried out in the presence of NaI:

| Cmpd No. | Compound Name | LC/MS | NMR (shifts in ppm) |
|---|---|---|---|
| 80 | 4-((2R,3S,4S,5R)-3-(2-(3- | ESI-MS m/z calc. | ¹H NMR (500 MHz, |
| | cyanocyclobutoxy)-3,4- | 538.16394, found 539.6 | Methanol-d₄) δ 8.49 (dd, |
| | difluorophenyl)-4,5-dimethyl-5- | (M + 1)⁺; 537.6 (M − 1)⁻; | J = 5.5, 3.7 Hz, 1H), 8.25 |
| | (trifluoromethyl)tetrahydrofuran- | Retention time: 3.29 | (t, J = 2.6 Hz, 1H), 7.91 |
| | 2-carboxamido)picolinamide | minutes | (dd, J = 5.6, 2.2 Hz, 1H), |
| | (Mixture of diastereomers at | | 7.14 (qd, J = 5.8, 2.9 Hz, |
| | 3-cyanocyclobutoxy group.) | | 1H), 7.05-6.96 (m, 1H), |
| | | | 5.08 (dd, J = 10.4, 1.9 Hz, |
| | | | 1H), 5.05-4.97 (m, 1H), |
| | | | 4.67 (qdd, J = 8.1, 5.2, |
| | | | 1.9 Hz, 1H), 4.36 (ddd, J = |
| | | | 10.3, 8.0, 4.7 Hz, 1H), |
| | | | 2.97-2.51 (m, 5H), 1.70- |
| | | | 1.64 (m, 3H), 0.82 (dp, J = |
| | | | 7.2, 2.3 Hz, 3H) ppm; |
| | | | amides NH and NH₂ not |
| | | | observed. |

The following compounds were made using the method described in Example 8, except that the conditions used in step 5 were those described in Example 5 step 2 using different alkylating agents:

| Cmpd No. | Compound Name | LC/MS | NMR (shifts in ppm) |
|---|---|---|---|
| 81 | 4-((2R,3S,4S,5R)-3-(3,4- | ESI-MS m/z calc. | ¹H NMR (500 MHz, |
| | difluoro-2-((S)-3-hydroxy-2- | 531.17926, found 532.4 | DMSO-d₆) δ 10.75 (s, |
| | methylpropoxy)phenyl)-4,5- | (M + 1)⁺; 530.3 (M − 1)⁻; | 1H), 8.48 (d, J = 5.5 Hz, |
| | dimethyl-5- | Retention time: 3.13 | 1H), 8.27 (d, J = 2.2 Hz, |
| | (trifluoromethyl)tetrahydrofuran- | minutes | 1H), 8.05 (d, J = 2.8 Hz, |
| | 2-carboxamido)picolinamide | | 1H), 7.81 (dd, J = 5.5, 2.2 |
| | | | Hz, 1H), 7.60 (d, J = 2.8 |
| | | | Hz, 1H), 7.15 (dd, J = |
| | | | 8.7, 4.1 Hz, 2H), 5.10 (d, |
| | | | J = 10.4 Hz, 1H), 4.61 (s, |
| | | | 1H), 4.29 (dd, J = 10.4, |
| | | | 7.5 Hz, 1H), 4.05 (dddd, J = |
| | | | 14.5, 9.0, 5.7, 1.6 Hz, |
| | | | 2H), 3.50-3.41 (m, 2H), |
| | | | 2.77 (p, J = 7.5 Hz, 1H), |
| | | | 1.99 (dq, J = 12.7, 6.3 Hz, |
| | | | 1H), 1.60 (s, 3H), 1.00 (d, |
| | | | J = 6.9 Hz, 3H), 0.75- |
| | | | 0.70 (m, 3H) ppm. |
| 82 | 4-((2R,3S,4S,5R)-3-(3,4- | ESI-MS m/z calc. | ¹H NMR (500 MHz, |
| | difluoro-2-((R)-3-hydroxy-2- | 531.17926, found 532.4 | DMSO-d₆) δ 10.74 (s, |
| | methylpropoxy)phenyl)-4,5- | (M + 1)⁺; 530.3 (M − 1)⁻; | 1H), 8.49 (d, J = 5.5 Hz, |
| | dimethyl-5- | Retention time: 3.12 | 1H), 8.28 (d, J = 2.2 Hz, |
| | (trifluoromethyl)tetrahydrofuran- | minutes | 1H), 8.05 (d, J = 2.8 Hz, |
| | 2-carboxamido)picolinamide | | 1H), 7.82 (dd, J = 5.5, 2.2 |
| | | | Hz, 1H), 7.60 (d, J = 2.8 |
| | | | Hz, 1H), 7.24-7.11 (m, |
| | | | 2H), 5.11 (d, J = 10.4 Hz, |
| | | | 1H), 4.62 (s, 1H), 4.30 |
| | | | (dd, J = 10.4, 7.5 Hz, |
| | | | 1H), 4.15 (ddd, J = 9.0, |
| | | | 5.3, 1.8 Hz, 1H), 3.92 |
| | | | (ddd, J = 8.2, 6.3, 1.4 Hz, |
| | | | 1H), 3.43 (d, J = 6.2 Hz, |
| | | | 2H), 2.76 (p, J = 7.5 Hz, |
| | | | 1H), 2.09-1.96 (m, 1H), |
| | | | 1.61 (s, 3H), 1.00 (d, J = |
| | | | 6.8 Hz, 3H), 0.75-0.70 |
| | | | (m, 3H) ppm. |

The following compounds were made using the method described in Example 8, except that the order in which steps 5 and 6 was carried out was reversed. The conditions used in step 5 were those described in Example 5 step 2, using different benzyl protected alkylating agents and performing the reaction at 50° C. A final deprotection step was carried out for 24 h at ambient temperature in the presence of a catalytic amount of Pd/C and an atmospheric pressure of hydrogen in EtOH as the solvent:

| Cmpd No. | Compound Name | LC/MS | NMR (shifts in ppm) |
|---|---|---|---|
| 83 | 4-((2R,3S,4S,5R)-3-(2-((1H- | ESI-MS m/z calc. | ¹H NMR (500 MHz, |
| | pyrazol-3-yl)methoxy)-3,4- | 539.1592, found 540.6 | Chloroform-d) δ 8.99 (s, |
| | difluorophenyl)-4,5-dimethyl-5- | (M + 1)⁺; Retention time: | 1H), 8.48 (d, J = 5.7 Hz, |
| | (trifluoromethyl)tetrahydrofuran- | 3.01 minutes | 1H), 8.20-7.98 (m, 3H), |
| | 2-carboxamido)picolinamide | | 7.40 (d, J = 2.3 Hz, 1H), |
| | | | 7.07 (dd, J = 8.7, 5.7 Hz, |
| | | | 1H), 6.94 (q, J = 8.8 Hz, |
| | | | 1H), 6.28 (d, J = 2.2 Hz, |
| | | | 1H), 6.08 (s, 1H), 5.32 (d, |
| | | | J = 12.1 Hz, 1H), 5.16 (d, |
| | | | J = 11.9 Hz, 1H), 4.94 (d, |
| | | | J = 11.3 Hz, 1H), 4.03 |
| | | | (dd, J = 11.3, 7.9 Hz, |
| | | | 1H), 2.70 (dq, J = 15.1, |
| | | | 7.3 Hz, 1H), 1.60 (s, 3H), |
| | | | 0.81-0.64 (m, 3H) ppm; |
| | | | NH amine not observed. |

### Example 9

### 4-((2R,3S,4S,5R)-3-(3,4-difluoro-2-(2-hydroxyethoxy)phenyl)-4,5-dimethyl-5-(trifluoromethyl)tetrahydrofuran-2-carboxamido)-2-(methylcarbamoyl)pyridine 1-oxide (84)

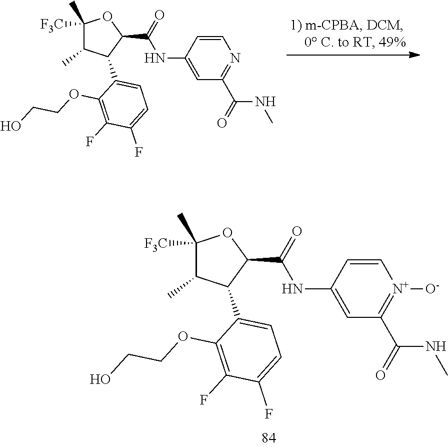 [SMILES: CNC(=O)c1cc(NC(=O)[C@@H]2O[C@@](C)(C(F)(F)F)[C@@H](C)[C@H]2c2ccc(F)c(F)c2OCCO)ccn1 ; CNC(=O)c1cc(NC(=O)[C@@H]2O[C@@](C)(C(F)(F)F)[C@@H](C)[C@H]2c2ccc(F)c(F)c2OCCO)cc[n+]1[O-]]

### Step 1:

A solution of 4-((2R,3S,4S,5R)-3-(3,4-difluoro-2-(2-hydroxyethoxy)phenyl)-4,5-dimethyl-5-(trifluoromethyl)tetrahydrofuran-2-carboxamido)-N-methylpicolinamide (40 mg, 0.07730 mmol) (38) in DCM (2 mL) was cooled down to 0° C. m-CPBA (45 mg, 0.2008 mmol) was added in one portion and the mixture was allowed to warm to ambient temperature. The reaction mixture was stirred at ambient temperature for 16 h. A further portion of m-CPBA (30 mg, 0.1339 mmol) was added and the mixture was stirred at ambient temperature for a further 4 h. A final portion of m-CPBA (30 mg, 0.1339 mmol) was added and the mixture was stirred at ambient temperature for another 3 h. The reaction mixture was diluted with EtOAc (10 mL) and poured over a saturated aqueous NaHCO₃ solution (10 mL). The aqueous layer was separated and extracted with EtOAc (2×10 mL). The organic extracts were combined, washed with brine (10 mL), dried over MgSO₄, filtered, and concentrated in vacuo. Purification by reverse phase HPLC using a X-bridge C18 column (150×19 mm, 5 mm particle size) from Waters (Gradient: 37.9% to 52.6% acetonitrile in water (supplemented with 0.1% ammonium hydroxide) over 9 minutes; Flow rate: 19 mL/min; sample diluted in acetonitrile and injected at 1 mL/min) gave 4-((2R,3S,4S,5R)-3-(3,4-difluoro-2-(2-hydroxyethoxy)phenyl)-4,5-dimethyl-5-(trifluoromethyl)tetrahydrofuran-2-carboxamido)-2-(methylcarbamoyl)pyridine 1-oxide (84, 20.0 mg, 49%) as a white solid. ¹H NMR (500 MHZ, DMSO-d₆) δ 11.31 (q, J=4.8 Hz, 1H), 10.79 (s, 1H), 8.53 (d, J=3.2 Hz, 1H), 8.32 (d, J=7.2 Hz, 1H), 7.90 (dd, J=7.2, 3.2 Hz, 1H), 7.19-7.13 (m, 2H), 5.10 (d, J=10.7 Hz, 1H), 4.95 (s, 1H), 4.40 (dd, J=10.7, 7.3 Hz, 1H), 4.16-4.06 (m, 2H), 3.73-3.67 (m, 2H), 2.94-2.88 (m, 1H), 2.88 (d, J=4.8 Hz, 3H), 1.61 (s, 3H), 0.73-0.69 (m, 3H) ppm.

### Example 10

### 4-((2R,3S,4S,5R)-3-(3,4-difluoro-2-(2-(1-methyl-1H-imidazol-4-yl)ethoxy)phenyl)-4,5-dimethyl-5-(trifluoromethyl)tetrahydrofuran-2-carboxamido) picolinamide (85)

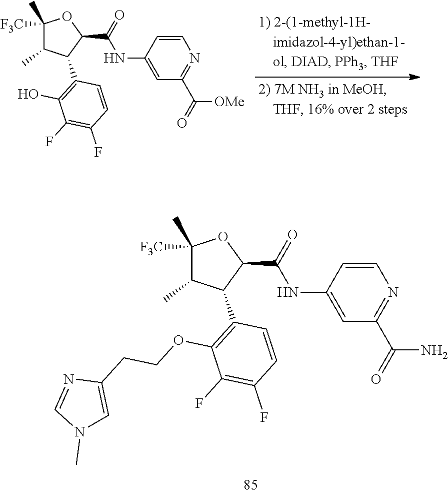 [SMILES: COC(=O)c1cc(NC(=O)[C@@H]2O[C@@](C)(C(F)(F)F)[C@@H](C)[C@H]2c2ccc(F)c(F)c2O)ccn1 ; C[C@H]1[C@@H](c2ccc(F)c(F)c2OCCc2cn(C)cn2)[C@H](C(=O)Nc2ccnc(C(N)=O)c2)O[C@@]1(C)C(F)(F)F]

### Step 1:

2-(1-Methyl-1H-imidazol-4-yl) ethan-1-ol (51 mg, 0.4043 mmol) and polymer-bound PPh₃ (134 mg of 3 mmol/g, 0.4020 mmol, 100-200 mesh, 2% cross-linked with divinylbenzene) were successively added to a solution of methyl 4-((2R,3S,4S,5R)-3-(3,4-difluoro-2-hydroxyphenyl)-4,5-dimethyl-5-(trifluoromethyl)tetrahydrofuran-2-carboxamido) picolinate (Product of Example 8, Step 4, 95 mg, 0.2003 mmol) in THF (2 mL). DIAD (80 μL, 0.4063 mmol) was then added dropwise to the suspension. The reaction was stirred at ambient temperature for 3 hours and 30 min to give a mixture containing methyl 4-((2R,3S,4S,5R)-3-(3,4-difluoro-2-(2-(1-methyl-1H-imidazol-4-yl)ethoxy)phenyl)-4,5-dimethyl-5-(trifluoromethyl)tetrahydrofuran-2-carboxamido) picolinate, which was used in the next step without further purification.

### Step 2:

Methanolic ammonia (1.4 mL of 7 M, 9.800 mmol) was added to a mixture containing methyl 4-((2R,3S,4S,5R)-3-(3,4-difluoro-2-(2-(1-methyl-1H-imidazol-4-yl)ethoxy)phenyl)-4,5-dimethyl-5-(trifluoromethyl)tetrahydrofuran-2-carboxamido) picolinate (116.7 mg, 0.2003 mmol) diluted with THF (2.0 mL). The reaction mixture was stirred at ambient temperature for 16 h. The reaction mixture was filtered to remove polymer supported PPh₃ from the previous step and then concentrated in vacuo. Methanolic ammonia (1.4 mL of 7 M, 9.800 mmol) was added and the resulting mixture was stirred at ambient temperature for 4 h. The reaction mixture was concentrated in vacuo. Purification by flash chromatography (12 g SiO₂, 40 to 100% EtOAc in Heptane then 0 to 5% EtOH in EtOAc) followed by reverse phase HPLC using a X-bridge C18 column (150×19 mm, 5 mm particle size) from Waters (Gradient: 37.9% to 52.6% acetonitrile in water (supplemented with 0.1% ammonium hydroxide) over 9 minutes; Flow rate: 19 mL/min; sample diluted in acetonitrile and injected at 1 mL/min) gave 4-((2R,3S,4S,5R)-3-(3,4-difluoro-2-(2-(1-methyl-1H-imidazol-4-yl)ethoxy)phenyl)-4,5-dimethyl-5-(trifluoromethyl)tetrahydrofuran-2-carboxamido) picolinamide (85, 17.9 mg, 16% over 2 steps) as a white solid. ¹H NMR (500 MHZ, DMSO-d₆) δ 10.69 (s, 1H), 8.49 (d, J=5.5 Hz, 1H), 8.29 (d, J=2.1 Hz, 1H), 8.05 (d, J=2.3 Hz, 1H), 7.83 (dd, J=5.5, 2.1 Hz, 1H), 7.61 (d, J=2.3 Hz, 1H), 7.50 (s, 1H), 7.17-7.10 (m, 2H), 6.95 (s, 1H), 5.06 (d, J=10.3 Hz, 1H), 4.46-4.39 (m, 1H), 4.36-4.30 (m, 1H), 4.15 (dd, J=10.3, 7.5 Hz, 1H), 3.57 (s, 3H), 2.91 (t, J=6.5 Hz, 2H), 2.60-2.53 (m, 1H), 1.50 (s, 3H), 0.66 (d, J=6.3 Hz, 3H) ppm. ESI-MS m/z calc. 567.19050, found 567.7 (M+1)⁺; 566.0 (M−1); Retention time: 3.10 minutes.

The following compounds were made using the method described in Example 10, except that different alcohols were used in step 1:

| Cmpd No. | Compound Name | LC/MS | NMR (shifts in ppm) |
|---|---|---|---|
| 86 | 4-((2R,3S,4S,5R)-3-(3,4- | ESI-MS m/z calc. | ¹H NMR (500 MHz, |
| | difluoro-2-(2-(1-methyl-1H- | 567.19050, found 567.9 | DMSO-d₆) δ 10.68 (s, |
| | imidazol-5-yl)ethoxy)phenyl)- | (M + 1)⁺; 566.0 (M − 1)⁻; | 1H), 8.49 (d, J = 5.4 Hz, |
| | 4,5-dimethyl-5- | Retention time: 3.04 | 1H), 8.29 (d, J = 2.2 Hz, |
| | (trifluoromethyl)tetrahydrofuran- | minutes | 1H), 8.06 (d, J = 2.2 Hz, |
| | 2-carboxamido)picolinamide | | 1H), 7.83 (dd, J = 5.4, 2.2 |
| | | | Hz, 1H), 7.61 (d, J = 2.2 |
| | | | Hz, 1H), 7.48 (s, 1H), |
| | | | 7.21-7.13 (m, 2H), 6.77 |
| | | | (d, J = 0.7 Hz, 1H), 5.07 |
| | | | (d, J = 10.2 Hz, 1H), 4.40- |
| | | | 4.34 (m, 1H), 4.32- |
| | | | 4.27 (m, 1H), 4.15 (dd, J = |
| | | | 10.2, 7.7 Hz, 1H), 3.56 |
| | | | (s, 3H), 3.05 (t, J = 6.5 |
| | | | Hz, 2H), 2.60-2.53 (m, |
| | | | 1H), 1.47 (s, 3H), 0.68 (d, |
| | | | J = 6.5 Hz, 3H) ppm. |
| 87 | 4-((2R,3S,4S,5R)-3-(3,4- | ESI-MS m/z calc. | ¹H NMR (500 MHz, |
| | difluoro-2-(2-(1-methyl-1H- | 567.51702, found 567.8 | DMSO-d₆) δ 10.63 (s, |
| | imidazol-2-yl)ethoxy)phenyl)- | (M + 1)⁺; 566.0 (M − 1)⁻; | 1H), 8.48 (d, J = 5.5 Hz, |
| | 4,5-dimethyl-5- | Retention time: 3.04 | 1H), 8.29 (d, J = 1.8 Hz, |
| | (trifluoromethyl)tetrahydrofuran- | minutes | 1H), 8.05 (d, J = 2.3 Hz, |
| | 2-carboxamido)picolinamide | | 1H), 7.83 (dd, J = 5.5, 2.2 |
| | | | Hz, 1H), 7.60 (d, J = 2.3 |
| | | | Hz, 1H), 7.18-7.12 (m, |
| | | | 2H), 6.98 (d, J = 1.2 Hz, |
| | | | 1H), 6.70 (d, J = 1.2 Hz, |
| | | | 1H), 5.06 (d, J = 10.3 Hz, |
| | | | 1H), 4.57-4.51 (m, 1H), |
| | | | 4.50-4.44 (m, 1H), 4.20 |
| | | | (dd, J = 10.3, 7.6 Hz, |
| | | | 1H), 3.57 (s, 3H), 3.11 (t, |
| | | | J = 6.3 Hz, 2H), 2.60- |
| | | | 2.53 (m, 1H), 1.52 (s, |
| | | | 3H), 0.65 (d, J = 6.4 Hz, |
| | | | 3H) ppm. |
| 88 | 4-((2R,3S,4S,5R)-3-(3,4- | ESI-MS m/z calc. | ¹H NMR (500 MHz, |
| | difluoro-2-((1-methyl-1H- | 553.17487, found 554.0 | DMSO-d₆) δ 10.64 (s, |
| | imidazol-4- | (M + 1)⁺; 552.0 (M − 1)⁻; | 1H), 8.49 (d, J = 5.5 Hz, |
| | yl)methoxy)phenyl)-4,5- | Retention time: 3.33 | 1H), 8.28 (d, J = 2.2 Hz, |
| | dimethyl-5- | minutes | 1H), 8.05 (d, J = 2.8 Hz, |
| | (trifluoromethyl)tetrahydrofuran- | | 1H), 7.81 (dd, J = 5.5, 2.2 |
| | 2-carboxamido)picolinamide | | Hz, 1H), 7.60 (d, J = 2.8 |
| | | | Hz, 1H), 7.49 (s, 1H), |
| | | | 7.20-7.07 (m, 3H), 5.03 |
| | | | (d, J = 10.7 Hz, 1H), 5.01- |
| | | | 4.97 (m, 2H), 4.15 (dd, J = |
| | | | 10.7, 7.3 Hz, 1H), 3.57 |
| | | | (s, 3H), 2.71-2.65 (m, |
| | | | 1H), 1.54 (s, 3H), 0.62 (d, |
| | | | J = 2.6 Hz, 3H) ppm. |
| 89 | 4-((2R,3S,4S,5R)-3-(3,4- | ESI-MS m/z calc. | ¹H NMR (500 MHz, |
| | difluoro-2-((1-methyl-1H- | 553.17487, found 554.0 | DMSO-d₆) δ 10.66 (s, |
| | imidazol-5- | (M + 1)⁺; 552.0 (M − 1)⁻; | 1H), 8.49 (d, J = 5.5 Hz, |
| | yl)methoxy)phenyl)-4,5- | Retention time: 3.28 | 1H), 8.27 (d, J = 1.8 Hz, |
| | dimethyl-5- | minutes | 1H), 8.05 (d, J = 2.8 Hz, |
| | (trifluoromethyl)tetrahydrofuran- | | 1H), 7.83 (dd, J = 5.5, 2.2 |
| | 2-carboxamido)picolinamide | | Hz, 1H), 7.66 (s, 1H), |
| | | | 7.61 (d, J = 2.8 Hz, 1H), |
| | | | 7.25-7.12 (m, 2H), 6.95 |
| | | | (s, 1H), 5.19 (s, 2H), 5.05 |
| | | | (d, J = 10.4 Hz, 1H), 4.11 |
| | | | (dd, J = 10.4, 7.5 Hz, |
| | | | 1H), 3.71 (s, 3H), 2.43- |
| | | | 2.35 (m, 1H), 1.46 (s, |
| | | | 3H), 0.67 (d, J = 6.4 Hz, |
| | | | 3H) ppm. |
| 90 | 4-((2R,3S,4S,5R)-3-(3,4- | ESI-MS m/z calc. | |
| | difluoro-2-(3-hydroxy-2- | 531.17926, found 532.2 | |
| | methylpropoxy)phenyl)-4,5- | (M + 1)⁺; 530.3 (M − 1)⁻; | |
| | dimethyl-5- | Retention time: 3.12 | |
| | (trifluoromethyl)tetrahydrofuran- | minutes | |
| | 2-carboxamido)picolinamide | | |
| | (Mixture of epimers at the | | |
| | 3-hydroxy-2-methylpropoxy | | |
| | group.) | | |
| 91 | 4-((2R,3S,4S,5R)-3-(3,4- | ESI-MS m/z calc. | ¹H NMR (500 MHz, |
| | difluoro-2-(3-hydroxy-2- | 531.17926, found 548.1 | DMSO-d₆) δ 10.72 (s, |
| | (hydroxymethyl)propoxy)phenyl)- | (M + 1)⁺; 546.2 (M − 1)⁻; | 1H), 8.50 (d, 1H), 8.28 |
| | 4,5-dimethyl-5- | Retention time: 2.69 | (d, 1H), 8.06 (d, 1H), |
| | (trifluoromethyl)tetrahydrofuran- | minutes | 7.84-7.82 (m, 1H), 7.61 |
| | 2-carboxamido)picolinamide | | (d, 1H), 7.16 (m, 2H), |
| | | | 5.13-5.11(d, 1H), 4.56 |
| | | | (m, 2H), 4.31-4.29 (m, |
| | | | 1H), 4.22-4.20 (m, 1H), |
| | | | 4.13-4.11(m, 1H), 3.59- |
| | | | 3.53 (m, 4H), 2.82-2.79 |
| | | | (m, 1H), 2.02-1.99 (m, |
| | | | 1H), 1.62 (s, 3H), 0.73 |
| | | | (m, 3H) ppm. |
| 92 | rel-4-((2R*,3S*,4S*,5R*)-3- | ESI-MS m/z calc. | ¹H NMR (500 MHz, |
| | (3,4-difluoro-2-(2- | 531.17926, found 546.1 | DMSO-d₆) δ 10.73 (s, |
| | (hydroxymethyl)butoxy)phenyl)- | (M + 1)⁺; 544.2 (M − 1)⁻; | 1H), 8.49 (d, 1H), 8.27 |
| | 4,5-dimethyl-5- | Retention time: 3.24 | (d, 1H), 8.06 (d, 1H), |
| | (trifluoromethyl)tetrahydrofuran- | minutes | 7.84-7.82 (m, 1H), 7.61 |
| | 2-carboxamido)picolinamide | | (d, 1H), 7.17-7.14 (m, |
| | | | 2H), 5.12-5.10 (d, 1H), |
| | | | 4.56 (m, 1H), 4.30-4.29 |
| | | | (m, 1H), 4.18-4.15 (m, |
| | | | 1H), 4.09-4.06 (m, 1H), |
| | | | 3.51 (m, 2H), 2.79-2.76 |
| | | | (m, 1H), 1.75-1.74 (m, |
| | | | 1H), 1.61 (s, 3H), 1.47- |
| | | | 1.44 (qd, 2H), 0.94 (t, |
| | | | 3H), 0.73 (m, 3H) ppm. |
| 93 | rel-4-((2R*,3S*,4S*,5R*)-3- | ESI-MS m/z calc. | ¹H NMR (500 MHz, |
| | (3,4-difluoro-2-(2- | 531.17926, found 546.1 | DMSO-d₆) δ 10.73 (s, |
| | (hydroxymethyl)butoxy)phenyl)- | (M + 1)⁺; 544.2 (M − 1)⁻; | 1H), 8.49 (d, 1H), 8.27 |
| | 4,5-dimethyl-5- | Retention time: 3.24 | (d, 1H), 8.06 (d, 1H), |
| | (trifluoromethyl)tetrahydrofuran- | minutes | 7.84-7.82 (m, 1H), 7.61 |
| | 2-carboxamido)picolinamide | | (d, 1H), 7.17-7.14 (m, |
| | | | 2H), 5.12-5.10 (d, 1H), |
| | | | 4.56 (m, 1H), 4.30-4.29 |
| | | | (m, 1H), 4.18-4.15 (m, |
| | | | 1H), 4.09-4.06 (m, 1H), |
| | | | 3.51 (m, 2H), 2.79-2.76 |
| | | | (m, 1H), 1.75-1.74 (m, |
| | | | 1H), 1.61 (s, 3H), 1.47- |
| | | | 1.44 (qd, 2H), 0.94 (t, |
| | | | 3H), 0.73 (m, 3H) ppm. |
| 94 | 4-((2R,3S,4S,5R)-3-(3,4- | ESI-MS m/z calc. | |
| | difluoro-2-((3- | 559.17418, found 560.0 | |
| | (hydroxymethyl)oxetan-3- | (M + 1)⁺; 558.1 (M − 1)⁻; | |
| | yl)methoxy)phenyl)-4,5- | Retention time: 2.83 | |
| | dimethyl-5- | minutes | |
| | (trifluoromethyl)tetrahydrofuran- | | |
| | 2-carboxamido)picolinamide | | |
| 95 | 4-((2R,3S,4S,5R)-3-(3,4- | ESI-MS m/z calc. | |
| | difluoro-2-((1- | 543.17926, found 544.0 | |
| | (hydroxymethyl)cyclopropyl)me- | (M + 1)⁺; 542.1 (M − 1)⁻; | |
| | thoxy)phenyl)-4,5-dimethyl-5- | Retention time: 3.09 | |
| | (trifluoromethyl)tetrahydrofuran- | minutes | |
| | 2-carboxamido)picolinamide | | |
| 96 | 4-((2R,3S,4S,5R)-3-(3,4- | ESI-MS m/z calc. | |
| | difluoro-2-(2-(methylamino)-2- | 530.15886, found 531.0 | |
| | oxoethoxy)phenyl)-4,5- | (M + 1)⁺; 529.1 (M − 1)⁻; | |
| | dimethyl-5- | Retention time: 2.72 | |
| | (trifluoromethyl)tetrahydrofuran- | minutes | |
| | 2-carboxamido)picolinamide | | |
| 97 | 4-((2R,3S,4S,5R)-3-(3,4- | ESI-MS m/z calc. | |
| | difluoro-2-((1- | 557.19491, found 558.0 | |
| | (hydroxymethyl)cyclobutyl)me- | (M + 1)⁺; 556.2 (M − 1)⁻; | |
| | thoxy)phenyl)-4,5-dimethyl-5- | Retention time: 3.30 | |
| | (trifluoromethyl)tetrahydrofuran- | minutes | |
| | 2-carboxamido)picolinamide | | |
| 98 | rel-4-((2R*,3S*,4S*,5R*)-3- | ESI-MS m/z calc. | ¹H NMR (500 MHz, |
| | (3,4-difluoro-2-(((15,2S)-2- | 543.17926, found 544.0 | DMSO-d₆) δ 8.50 (dd, |
| | hydroxycyclobutyl)methoxy)phenyl)- | (M + 1)⁺; 542.1 (M − 1)⁻; | 1H), 8.27 (dd, 1H), 7.90 |
| | 4,5-dimethyl-5- | Retention time: 3.14 | (dd, 1H), 7.18-7.12 (m, |
| | (trifluoromethyl)tetrahydrofuran- | minutes | 1H), 7.03-6.95 (m, 1H), |
| | 2-carboxamido)picolinamide | | 5.09-5.07(d, 1H), 4.58- |
| | | | 4.52 (m, 1H), 4.47-4.42 |
| | | | (m, 2H), 4.24 (m, 1H), |
| | | | 2.89-2.82 (m, 2H), 2.32- |
| | | | 2.26 (m, 1H), 2.11-2.02 |
| | | | (m, 1H), 2.86-2.82 (m, |
| | | | 1H), 2.76-2.72 9m, 1H), |
| | | | 1.68 (s, 3H), 0.84-0.82 |
| | | | (m, 3H) ppm; amides NH |
| | | | and NH₂ and alcohol OH |
| | | | not observed. |
| 99 | rel-4-((2R*,3S*,4S*,5R*)-3- | ESI-MS m/z calc. | ¹H NMR (500 MHz, |
| | (3,4-difluoro-2-(((1R,2R)-2- | 543.17926, found 544.1 | DMSO-d₆) δ 8.50 (dd, |
| | hydroxycyclobutyl)methoxy)phenyl)- | (M + 1)⁺; 542.2 (M − 1)⁻; | 1H), 8.29 (dd, 1H), 7.90 |
| | 4,5-dimethyl-5- | Retention time: 3.27 | (dd, 1H), 7.14 (m, 1H), |
| | (trifluoromethyl)tetrahydrofuran- | minutes | 7.03-6.95 (m, 1H), 5.09- |
| | 2-carboxamido)picolinamide | | 5.07(d, 1H), 4.46-4.37 |
| | | | (m, 4H), 2.89-2.82 (m, |
| | | | 2H), 2.32-2.26 (m, 1H), |
| | | | 2.11-2.02 (m, 1H), 2.86- |
| | | | 2.82 (m, 1H), 2.76-2.72, |
| | | | m, 1H), 1.68 (s, 3H), |
| | | | 0.84-0.82 (m, 3H) ppm; |
| | | | amides NH and NH₂ and |
| | | | alcohol OH not observed. |
| 100 | 4-((2R,3S,4S,5R)-3-(3,4- | ESI-MS m/z calc. | ¹H NMR (500 MHz, |
| | difluoro-2-(3-hydroxy-3- | 545.1949, found 546.0 | Methanol-d₄) δ 8.49 (d, J = |
| | methylbutoxy)phenyl)-4,5- | (M + 1)⁺; 544.1 (M − 1)⁻; | 5.5 Hz, 1H), 8.25 (d, J = |
| | dimethyl-5- | Retention time: 3.04 | 2.1 Hz, 1H), 7.88 (dd, J = |
| | (trifluoromethyl)tetrahydrofuran- | minutes | 5.5, 2.2 Hz, 1H), 7.13 |
| | 2-carboxamido)picolinamide | | (ddd, J = 8.3, 5.7, 2.0 Hz, |
| | | | 1H), 7.01 (td, J = 9.3, 7.5 |
| | | | Hz, 1H), 5.05 (d, J = 10.5 |
| | | | Hz, 1H), 4.46 (dd, J = |
| | | | 10.5, 7.9 Hz, 1H), 3.83 (t, |
| | | | J = 7.3 Hz, 2H), 2.78 (p, J = |
| | | | 7.6 Hz, 1H), 2.14-2.01 |
| | | | (m, 2H), 1.66 (s, 3H), |
| | | | 1.38-1.35 (m, 6H), 0.77 |
| | | | (dt, J = 7.4, 2.3 Hz, 3H) |
| | | | ppm; amides NH and |
| | | | NH₂ and alcohol OH not |
| | | | observed. |

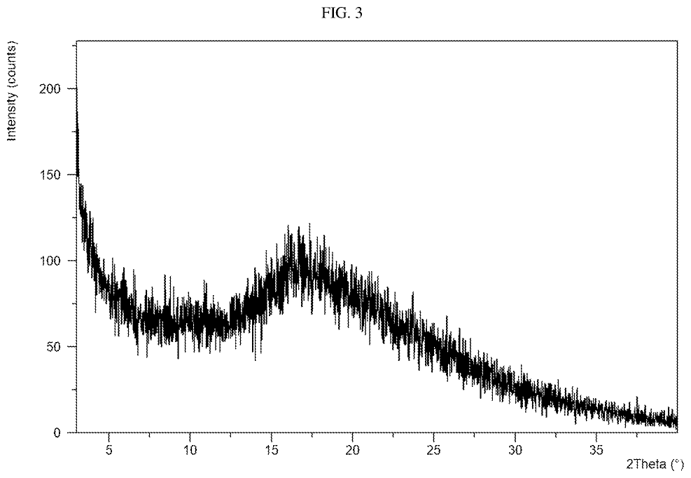

Compound 86 was analyzed by X-ray powder diffraction and determined to be amorphous (see FIG. 3).

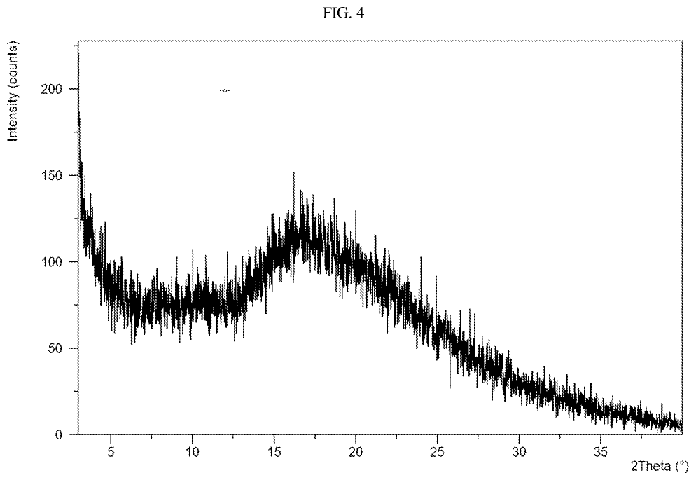

Compound 87 was analyzed by X-ray powder diffraction and determined to be amorphous (see FIG. 4).

### Example 11

### 4-((2R,3S,4S,5R)-3-(3,4-difluoro-2-((1-methylazetidin-3-yl)oxy)phenyl)-4,5-dimethyl-5-(trifluoromethyl)tetrahydrofuran-2-carboxamido) picolinamide (101)

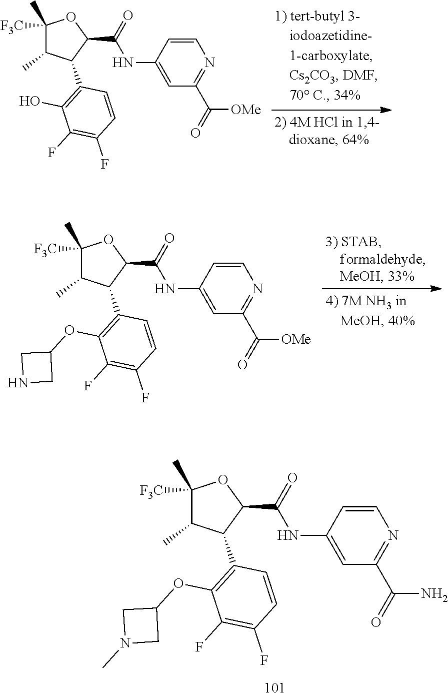 [SMILES: COC(=O)c1cc(NC(=O)[C@@H]2O[C@@](C)(C(F)(F)F)[C@@H](C)[C@H]2c2ccc(F)c(F)c2O)ccn1 ; COC(=O)c1cc(NC(=O)[C@@H]2O[C@@](C)(C(F)(F)F)[C@@H](C)[C@H]2c2ccc(F)c(F)c2OC2CNC2)ccn1 ; C[C@H]1[C@@H](c2ccc(F)c(F)c2OC2CN(C)C2)[C@H](C(=O)Nc2ccnc(C(N)=O)c2)O[C@@]1(C)C(F)(F)F]

### Step 1:

Cesium carbonate (144 mg, 0.4420 mmol) was added to a solution of methyl 4-((2R,3S,4S,5R)-3-(3,4-difluoro-2-hydroxyphenyl)-4,5-dimethyl-5-(trifluoromethyl)tetrahydrofuran-2-carboxamido) picolinate (Product of Example 8, Step 4,150 mg, 0.316 mmol) and tert-butyl 3-iodoazetidine-1-carboxylate (268 mg, 0.9466 mmol) in DMF (1 mL). The reaction mixture was heated to 70° C. overnight. The mixture was cooled down to ambient temperature and partitioned between MTBE (20 ml) and water (20 ml). The aqueous layer was separated and extracted with MTBE (10 mL). The combined organic extracts were washed with brine (1×10 mL), dried over MgSO₄, filtered, and concentrated in vacuo. Purification by flash chromatography (12 g SiO₂, 0 to 100% EtOAc in heptane) gave methyl 4-((2R,3S,4S,5R)-3-(2-((1-(tert-butoxycarbonyl) azetidin-3-yl)oxy)-3,4-difluorophenyl)-4,5-dimethyl-5-(trifluoromethyl)tetrahydrofuran-2-carboxamido) picolinate (130 mg, 34%) as a white solid. ESI-MS m/z calc. 629.21606, found 630.4 (M+1)⁺; 628.3 (M−1); Retention time: 1.07 minutes.

### Step 2:

4 M HCl in 1,4-dioxane (953 μL, 3.812 mmol) was added to a solution of methyl 4-((2R,3S,4S,5R)-3-(2-((1-(tert-butoxycarbonyl) azetidin-3-yl)oxy)-3,4-difluorophenyl)-4,5-dimethyl-5-(trifluoromethyl)tetrahydrofuran-2-carboxamido) picolinate (120 mg, 0.1906 mmol) in 1,4-dioxane (1 mL). The reaction mixture was stirred at ambient temperature for 3 h, and the mixture was then concentrated in vacuo. The residual solid was partitioned between DCM (3 mL) and sodium carbonate (5 mL of 1 M, 5 mmol). The aqueous layer was separated and extracted with DCM (3 mL). The combined organic extracts were washed with brine (1×10 mL), dried over MgSO₄, filtered, and concentrated in vacuo to give methyl 4-((2R,3S,4S,5R)-3-(2-(azetidin-3-yloxy)-3,4-difluorophenyl)-4,5-dimethyl-5-(trifluoromethyl)tetrahydrofuran-2-carboxamido) picolinate (80 mg, 64%), which was used as such in the next step. ESI-MS m/z calc. 529.16364, found 530.3 (M+1)⁺; 528.2 (M−1); Retention time: 0.83 minutes.

### Step 3:

Formaldehyde (112 μL, 4.066 mmol) and STAB (128 mg, 0.6039 mmol) were successively added to a solution of methyl 4-((2R,3S,4S,5R)-3-(2-(azetidin-3-yloxy)-3,4-difluorophenyl)-4,5-dimethyl-5-(trifluoromethyl)tetrahydrofuran-2-carboxamido) picolinate (80 mg, 0.1511 mmol) in methanol (2 mL). The reaction mixture was stirred for 4 h at ambient temperature. The mixture was concentrated in vacuo and partitioned between MTBE (5 ml) and a 2 M sodium carbonate solution (5 ml). The aqueous phase was separated and extracted with MTBE (10 mL). The combined organic extracts were washed with brine (1×10 mL), dried over MgSO₄, filtered, and concentrated to dryness to give methyl 4-((2R,3S,4S,5R)-3-(3,4-difluoro-2-((1-methylazetidin-3-yl)oxy)phenyl)-4,5-dimethyl-5-(trifluoromethyl)tetrahydrofuran-2-carboxamido) picolinate (55 mg, 33%), which was used as is in the next step. ESI-MS m/z calc. 543.17926, found 544.3 (M+1)⁺; 542.2 (M−1); Retention time: 0.92 minutes.

### Step 4:

Methanolic ammonia (322 μL of 7 M, 2.254 mmol) was added to a solution of methyl 4-((2R,3S,4S,5R)-3-(3,4-difluoro-2-((1-methylazetidin-3-yl)oxy)phenyl)-4,5-dimethyl-5-(trifluoromethyl)tetrahydrofuran-2-carboxamido) picolinate (40 mg, 0.07360 mmol) in methanol (1 mL). The reaction mixture was stirred overnight at ambient temperature, and then concentrated in vacuo. Purification by reverse phase preparative HPLC gave 4-((2R,3S,4S,5R)-3-(3,4-difluoro-2-((1-methylazetidin-3-yl)oxy)phenyl)-4,5-dimethyl-5-(trifluoromethyl)tetrahydrofuran-2-carboxamido) picolinamide (101, 16 mg, 40%). ¹H NMR (500 MHZ, DMSO-d₆) δ 10.70 (s, 1H), 8.49 (d, J=5.5 Hz, 1H), 8.30-8.25 (m, 1H), 8.05 (s, 1H), 7.83 (dd, J=5.5, 2.2 Hz, 1H), 7.60 (s, 1H), 7.21-7.14 (m, 2H), 5.11 (d, J=10.3 Hz, 1H), 4.76 (td, J=5.5, 2.5 Hz, 1H), 4.32 (dd, J=10.3, 7.4 Hz, 1H), 3.61 (t, J=6.9 Hz, 1H), 3.53 (t, J=6.9 Hz, 1H), 3.13 (dd, J=7.8, 5.0 Hz, 1H), 3.06 (dd, J=7.8, 5.0 Hz, 1H), 2.78 (p, J=7.5 Hz, 1H), 2.24 (s, 3H), 1.63 (s, 3H), 0.73 (d, J=7.3 Hz, 3H) ppm. ESI-MS m/z calc. 528.1796, found 529.3 (M+1)⁺; 527.2 (M−1); Retention time: 3.05 minutes.

The following compounds were made using the method described in Example 11, except that different alkylating agent were used in step 1:

| Cmpd No. | Compound Name | LC/MS | NMR (shifts in ppm) |
|---|---|---|---|
| 102 | rel-4-((2R*,3S*,4S*,5R*)-3- | ESI-MS m/z calc. | ¹H NMR (500 MHz, |
| | (3,4-difluoro-2-((-1- | 542.19525, found 543.3 | DMSO-d₆) δ 10.68 (s, |
| | methylpyrrolidin-3- | (M + 1)⁺; 541.2 (M − 1)⁻; | 1H), 8.50 (d, J = 5.4 Hz, |
| | yl)oxy)phenyl)-4,5-dimethyl-5- | Retention time: 3.21 | 1H), 8.31-8.26 (m, 1H), |
| | (trifluoromethyl)tetrahydrofuran- | minutes | 8.06 (d, J = 2.7 Hz, 1H), |
| | 2-carboxamido)picolinamide | | 7.86-7.80 (m, 1H), 7.61 |
| | | | (d, J = 2.7 Hz, 1H), 7.16 |
| | | | (dd, J = 8.4, 5.4 Hz, 2H), |
| | | | 5.11 (d, J = 10.8 Hz, 1H), |
| | | | 5.04-5.00 (m, 1H),4.32 |
| | | | (dd, J = 10.8, 7.2 Hz, |
| | | | 1H), 2.90-2.72 (m, 3H), |
| | | | 2.56 (dd, J = 10.8, 5.2 Hz, |
| | | | 1H), 2.24 (s, 3H), 2.23- |
| | | | 2.20 (m, 1H), 2.06 (dtd, J = |
| | | | 13.6, 7.9, 5.2 Hz, 1H), |
| | | | 1.78 (dt, J = 14.5, 7.2 Hz, |
| | | | 1H), 1.63 (s, 3H), 0.75- |
| | | | 0.69 (m, 3H) ppm. |
| 103 | rel-4-((2R*,3S*,4S*,5R*)-3- | ESI-MS m/z calc. | ¹H NMR (500 MHz, |
| | (3,4-difluoro-2-((-1- | 542.19525, found 543.3 | Chloroform-d) δ 8.79- |
| | methylpyrrolidin-3- | (M + 1)⁺; 541.2 (M − 1)⁻; | 8.69 (m, 1H), 8.38 (d, J = |
| | yl)oxy)phenyl)-4,5-dimethyl-5- | Retention time: 3.30 | 5.5 Hz, 1H), 8.12-8.03 |
| | (trifluoromethyl)tetrahydrofuran- | minutes | (m, 1H), 7.92 (s, 1H), |
| | 2-carboxamido)picolinamide | | 7.76 (d, J = 4.5 Hz, 1H), |
| | | | 7.04 (ddd, J = 8.1, 5.4, |
| | | | 2.0 Hz, 1H), 6.86 (td, J = |
| | | | 9.3, 7.5 Hz, 1H), 5.57 (d, |
| | | | J = 4.4 Hz, 1H), 5.03- |
| | | | 4.89 (m, 2H), 4.50 (d, J = |
| | | | 10.1 Hz, 1H), 3.0-2.9 (m, |
| | | | 1H), 2.77 (d, J = 7.3 Hz, |
| | | | 2H), 2.27 (s, 1H), 2.04 (s, |
| | | | 6H), 1.64 (s, 3H), 0.67 |
| | | | (dq, J = 7.5, 2.4 Hz, 3H) |
| | | | ppm. |
| 104 | 4-((2R,3S,4S,5R)-3-(3,4- | ESI-MS m/z calc. | ¹H NMR (500 MHz, |
| | difluoro-2-((1-methylazetidin- | 542.19525, found 543.3 | Methanol-d₄) δ 8.53 (dd, |
| | 3-yl)methoxy)phenyl)-4,5- | (M + 1)⁺; 541.2 (M − 1)⁻; | J = 5.5, 0.6 Hz, 1H), 8.43 |
| | dimethyl-5- | Retention time: 3.06 | (dd, J = 8.6, 2.2 Hz, 1H), |
| | (trifluoromethyl)tetrahydrofuran- | minutes | 7.80 (ddd, J = 8.0, 5.6, |
| | 2-carboxamido)picolinamide | | 2.2 Hz, 1H), 7.21 (qd, J = |
| | (Trifluoroacetate salt) | | 7.3, 6.1, 3.8 Hz, 1H), 7.17- |
| | | | 7.04 (m, 1H), 5.11 (t, J = |
| | | | 9.6 Hz, 1H), 4.56-4.48 |
| | | | (m, 1H), 4.48-4.41 (m, |
| | | | 2H), 4.36 (h, J = 5.0 Hz, |
| | | | 1H), 4.31-4.16 (m, 2H), |
| | | | 4.11 (ddd, J = 21.7, 10.9, |
| | | | 8.7 Hz, 1H), 3.40 (ddt, J = |
| | | | 14.5, 8.8, 3.1 Hz, 1H), |
| | | | 2.99 (d, J = 14.3 Hz, 3H), |
| | | | 2.81 (p, J = 7.9 Hz, 1H), |
| | | | 1.71-1.64 (m, 3H), 0.88 |
| | | | (dt, J = 7.4, 2.3 Hz, 3H) |
| | | | ppm; amides NH and |
| | | | NH₂ not observed. |
| 105 | 4-((2R,3S,4S,5R)-3-(3,4- | ESI-MS m/z calc. | ¹H NMR (500 MHz, |
| | difluoro-2-((3-fluoro-1- | 560.18585, found 561.0 | DMSO-d₆) δ 10.69 (s, |
| | methylazetidin-3- | (M + 1)⁺; 559.0 (M − 1)⁻; | 1H), 8.50 (d, J = 5.5 Hz, |
| | yl)methoxy)phenyl)-4,5- | Retention time: 3.27 | 1H), 8.29 (d, J = 2.2 Hz, |
| | dimethyl-5- | minutes | 1H), 8.06 (d, J = 2.8 Hz, |
| | (trifluoromethyl)tetrahydrofuran- | | 1H), 7.83 (dd, J = 5.5, 2.2 |
| | 2-carboxamido)picolinamide | | Hz, 1H), 7.61 (s, 1H), |
| | | | 7.21 (dd, J = 8.9, 6.4 Hz, |
| | | | 2H), 5.13 (d, J = 10.6 Hz, |
| | | | 1H), 4.51 (dd, J = 24.8, |
| | | | 11.5 Hz, 1H), 4.38 (dd, J = |
| | | | 24.4, 11.4 Hz, 1H), |
| | | | 4.26 (dd, J = 10.5, 7.4 Hz, |
| | | | 1H), 3.52 (dt, J = 37.9, |
| | | | 10.2 Hz, 2H), 3.11 (ddd, |
| | | | J = 21.9, 13.7, 8.3 Hz, |
| | | | 2H), 2.73 (q, J = 7.5 Hz, |
| | | | 1H), 2.29 (s, 3H), 1.59 (s, |
| | | | 3H), 0.75-0.70 (m, 3H) |
| | | | ppm. |

The following compound was made using the method described in Example 11, except that a different alkylating agent was used in step 1. Step 3 was not required:

| Cmpd No. | Compound Name | LC/MS | NMR (shifts in ppm) |
|---|---|---|---|
| 106 | 4-((2R,3S,4S,5R)-3-(3,4- | ESI-MS m/z calc. | ¹H NMR (500 MHz, |
| | difluoro-2-((3-fluoroazetidin-3- | 546.17017, found 547.2 | DMSO-d₆) δ 10.72 (s, |
| | yl)methoxy)phenyl)-4,5- | (M + 1)⁺; 545.2 (M − 1)⁻; | 1H), 8.50 (d, J = 5.5 Hz, |
| | dimethyl-5- | Retention time: 2.89 | 1H), 8.28 (d, J = 2.1 Hz, |
| | (trifluoromethyl)tetrahydrofuran- | minutes | 1H), 8.06 (d, J = 2.6 Hz, |
| | 2-carboxamido)picolinamide | | 1H), 7.83 (dd, J = 5.5, 2.2 |
| | | | Hz, 1H), 7.61 (s, 1H), |
| | | | 7.20 (dd, J = 10.3, 6.6 Hz, |
| | | | 2H), 5.13 (d, J = 10.6 Hz, |
| | | | 1H), 4.56 (dd, J = 24.4, |
| | | | 11.5 Hz, 1H), 4.43 (dd, J = |
| | | | 23.8, 11.4 Hz, 1H), |
| | | | 4.27 (dd, J = 10.6, 7.3 Hz, |
| | | | 1H), 3.65 (dt, J = 20.0, |
| | | | 9.9 Hz, 2H), 3.50 (dt, J = |
| | | | 31.7, 11.0 Hz, 2H), 3.28 |
| | | | (s, 1H), 2.74 (q, J = 7.5 |
| | | | Hz, 1H), 1.58 (s, 3H), |
| | | | 0.72 (d, J = 7.5 Hz, 3H) |
| | | | ppm. |

### Example 12

### 4-((2R,3S,4S,5R)-3-(3,4-difluoro-2-(2-(3-fluoroazetidin-1-yl)ethoxy)phenyl)-4,5-dimethyl-5-(trifluoromethyl)tetrahydrofuran-2-carboxamido) picolinamide (107)

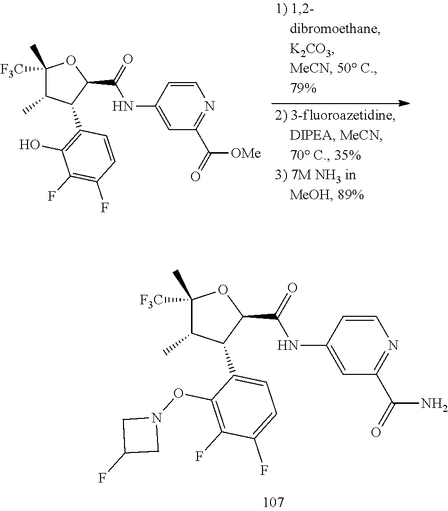 [SMILES: COC(=O)c1cc(NC(=O)[C@@H]2O[C@@](C)(C(F)(F)F)[C@@H](C)[C@H]2c2ccc(F)c(F)c2O)ccn1 ; C[C@H]1[C@@H](c2ccc(F)c(F)c2ON2CC(F)C2)[C@H](C(=O)Nc2ccnc(C(N)=O)c2)O[C@@]1(C)C(F)(F)F]

### Step 1:

1,2-Dibromoethane (1.68 mL, 19.50 mmol) was added to a stirred suspension of methyl 4-((2R,3S,4S,5R)-3-(3,4-difluoro-2-hydroxyphenyl)-4,5-dimethyl-5-(trifluoromethyl)tetrahydrofuran-2-carboxamido) picolinate (Product of Example 8, Step 4, 1.85 g, 3.900 mmol) and K₂CO₃ (811 mg, 5.868 mmol) in MeCN (5.50 mL). The reaction mixture was stirred at 50° C. overnight. The reaction mixture was then diluted with EtOAc (20 mL) and poured over water (20 mL). The aqueous layer was separated and extracted with EtOAc (2×20 mL). The combined organic extracts were washed with brine (30 mL), dried over MgSO₄, filtered, and concentrated in vacuo. Purification by flash chromatography (80 g SiO₂, 0 to 100% EtOAc in heptane) gave methyl 4-((2R,3S,4S,5R)-3-(2-(2-bromoethoxy)-3,4-difluorophenyl)-4,5-dimethyl-5-(trifluoromethyl)tetrahydrofuran-2-carboxamido) picolinate (1.781 g, 79%) as a white foam. ¹H NMR (500 MHz, Chloroform-d) δ 8.62 (d, J=5.5 Hz, 1H), 8.58 (s, 1H), 8.08 (d, J=2.2 Hz, 1H), 7.90 (dd, J=5.5, 2.2 Hz, 1H), 7.16-7.10 (m, 1H), 6.99-6.92 (m, 1H), 5.01 (d, J=11.3 Hz, 1H), 4.71-4.65 (m, 1H), 4.38 (dd, J=11.3, 7.6 Hz, 1H), 4.38-4.32 (m, 1H), 4.00 (s, 3H), 3.66 (ddd, J=11.2, 7.4, 3.5 Hz, 1H), 3.60 (ddd, J=11.2, 5.9, 3.5 Hz, 1H), 2.90-2.83 (m, 1H), 1.73 (s, 3H), 0.82-0.78 (m, 3H) ppm. ESI-MS m/z calc. 580.06323, found 582.7 (M+1)⁺; 580.8 (M−1); Retention time: 3.48 minutes.

### Step 2:

3-Fluoroazetidine (16 mg, 0.2131 mmol) and DIPEA (22 μL, 0.1263 mmol) were successively added to a solution of methyl 4-((2R,3S,4S,5R)-3-(2-(2-bromoethoxy)-3,4-difluorophenyl)-4,5-dimethyl-5-(trifluoromethyl)tetrahydrofuran-2-carboxamido) picolinate (25 mg, 0.04301 mmol) in MeCN (1 mL). The reaction mixture was heated to 70° C. overnight. The mixture was cooled to ambient temperature and partitioned between MTBE (5 ml) and water (5 ml). The aqueous layer was separated and extracted with MTBE (5 mL). The combined organic extracts were washed with brine (1×10 mL), dried over MgSO₄, filtered, and concentrated to in vacuo to give methyl 4-((2R,3S,4S,5R)-3-(3,4-difluoro-2-(2-(3-fluoroazetidin-1-yl)ethoxy)phenyl)-4,5-dimethyl-5-(trifluoromethyl)tetrahydrofuran-2-carboxamido) picolinate (12 mg, 35%), which was used as such in the next step. ESI-MS m/z calc. 575.1855, found 576.3 (M+1)⁺; Retention time: 1.73 minutes.

### Step 3:

Methanolic ammonia (297 μL of 7 M, 2.079 mmol) was added to a solution of methyl 4-((2R,3S,4S,5R)-3-(3,4-difluoro-2-(2-(3-fluoroazetidin-1-yl)ethoxy)phenyl)-4,5-dimethyl-5-(trifluoromethyl)tetrahydrofuran-2-carboxamido) picolinate (100 mg, 0.070 mmol) in methanol (1 mL). The reaction mixture was stirred at ambient temperature for 14 h. The mixture was then concentrated in vacuo. Purification by reverse phase HPLC using a Sunfire C18 column (150×19 mm, 5 mm particle size) from Waters (Gradient: 10% to 94.5% acetonitrile in water (supplemented with 0.05% trifluoroacetic acid) over 16 minutes; Flow rate: 19 mL/min; sample dissolved in acetonitrile and injected at 1 mL/min) gave 4-((2R,3S,4S,5R)-3-(3,4-difluoro-2-(2-(3-fluoroazetidin-1-yl)ethoxy)phenyl)-4,5-dimethyl-5-(trifluoromethyl)tetrahydrofuran-2-carboxamido) picolinamide (107, 36 mg, 89%), as a white powder. ¹H NMR (500 MHZ, DMSO-d₆) δ 10.70 (s, 1H), 8.49 (d, J=5.6 Hz, 1H), 8.29 (d, J=2.2 Hz, 1H), 8.05 (d, J=2.7 Hz, 1H), 7.85 (dd, J=5.5, 2.2 Hz, 1H), 7.63-7.58 (m, 1H), 7.17 (dd, J=8.5, 4.6 Hz, 2H), 5.18-5.10 (m, 2H), 4.44 (dd, J=10.6, 7.3 Hz, 1H), 4.15 (dt, J=10.2, 4.8 Hz, 1H), 4.04-3.95 (m, 1H), 3.64-3.51 (m, 2H), 3.23-3.06 (m, 2H), 2.91-2.72 (m, 3H), 1.67 (s, 3H), 0.74-0.68 (m, 3H) ppm. ESI-MS m/z calc. 560.18585, found 561.1 (M+1)⁺; 559.2 (M−1); Retention time: 3.21 minutes.

The following compounds were made using the method described in Example 12, except that the conditions used in step 2 were those described in Example 5 step 2, using different amines. Compound 123 was isolated as an impurity produced in the synthesis of compound 126:

| Cmpd No. | Compound Name | LC/MS | NMR (shifts in ppm) |
|---|---|---|---|
| 108 | 4-((2R,3S,4S,5R)-3-(2-(2-(1,4- | ESI-MS m/z calc. | ¹H NMR (500 MHz, |
| | oxazepan-4-yl)ethoxy)-3,4- | 586.22144, found 587.3 | DMSO-d₆) δ 10.67 (s, |
| | difluorophenyl)-4,5-dimethyl-5- | (M + 1)⁺; 585.2 (M − 1)⁻; | 1H), 8.49 (d, J = 5.5 Hz, |
| | (trifluoromethyl)tetrahydrofuran- | Retention time: 3.18 | 1H), 8.30 (d, J = 2.1 Hz, |
| | 2-carboxamido)picolinamide | minutes | 1H), 8.08-8.03 (m, 1H), |
| | | | 7.85 (dd, J = 5.4, 2.2 Hz, |
| | | | 1H), 7.60 (d, J = 2.9 Hz, |
| | | | 1H), 7.21-7.10 (m, 2H), |
| | | | 5.13 (d, J = 10.4 Hz, 1H), |
| | | | 4.39-4.24 (m, 2H), 4.21- |
| | | | 4.13 (m, 1H), 3.57 (t, J = |
| | | | 6.0 Hz, 2H), 3.54-3.48 |
| | | | (m, 2H), 2.89-2.77 (m, |
| | | | 3H), 2.73-2.63 (m, 4H), |
| | | | 1.70 (p, J = 5.9 Hz, 2H), |
| | | | 1.64 (s, 3H), 0.75-0.69 |
| | | | (m, 3H) ppm. |
| 109 | 4-((2R,3S,4S,5R)-3-(3,4- | ESI-MS m/z calc. | ¹H NMR (500 MHz, |
| | difluoro-2-(2-((R)-2- | 586.22144, found 587.3 | DMSO-d₆) δ 10.72 (s, |
| | methylmorpholino)ethoxy)phenyl)- | (M + 1)⁺; 585.2 (M − 1)⁻; | 1H), 8.40 (s, 1H), 8.21 (s, |
| | 4,5-dimethyl-5- | Retention time: 3.25 | 1H), 8.01 (s, 1H), 7.77 (s, |
| | (trifluoromethyl)tetrahydrofuran- | minutes | 1H), 7.53 (s, 1H), 7.22- |
| | 2-carboxamido)picolinamide | | 7.08 (m, 2H), 5.07 (d, J = |
| | | | 10.4 Hz, 1H), 4.32 (t, J = |
| | | | 8.4 Hz, 2H), 4.22 (ddt, J = |
| | | | 11.3, 5.9, 3.0 Hz, 1H), |
| | | | 3.71-3.59 (m, 1H), 3.46- |
| | | | 3.37 (m, 2H), 2.89- |
| | | | 2.74 (m, 2H), 2.74-2.61 |
| | | | (m, 3H), 1.98 (td, J = |
| | | | 11.3, 3.3 Hz, 1H), 1.70 |
| | | | (dd, J = 11.2, 9.9 Hz, |
| | | | 1H), 1.63 (s, 3H), 0.98 (d, |
| | | | J = 6.3 Hz, 3H), 0.70 (d, |
| | | | J = 7.5 Hz, 3H) ppm. |
| 110 | 4-((2R,3S,4S,5R)-3-(2-(2-(6- | ESI-MS m/z calc. | ¹H NMR (500 MHz, |
| | oxa-3-azabicyclo[3.1.1]heptan- | 584.2058, found 585.3 | DMSO-d₆) δ 10.68 (s, |
| | 3-yl)ethoxy)-3,4- | (M + 1)⁺; 583.2 (M − 1)⁻; | 1H), 8.50 (d, J = 5.5 Hz, |
| | difluorophenyl)-4,5-dimethyl-5- | Retention time: 3.15 | 1H), 8.30 (d, J = 2.1 Hz, |
| | (trifluoromethyl)tetrahydrofuran- | minutes | 1H), 8.06 (d, J = 2.7 Hz, |
| | 2-carboxamido)picolinamide | | 1H), 7.83 (dd, J = 5.5, 2.2 |
| | (Mixture of diastereomers at the | | Hz, 1H), 7.61 (d, J = 2.9 |
| | 6-oxa-3-azabicyclo[3.1.1] | | Hz, 1H), 7.19-7.12 (m, |
| | heptan-3-yl)ethoxy group.) | | 2H), 5.13 (d, J = 10.3 Hz, |
| | | | 1H), 4.41-4.23 (m, 4H), |
| | | | 3.10-2.78 (m, 6H), 2.73- |
| | | | 2.58 (m, 3H), 2.02 (d, J = |
| | | | 7.7 Hz, 1H), 1.61 (s, |
| | | | 3H), 0.74-0.68 (m, 3H) |
| | | | ppm. |
| 111 | 4-((2R,3S,4S,5R)-3-(3,4- | ESI-MS m/z calc. | ¹H NMR (500 MHz, |
| | difluoro-2-(2-((S)-2- | 586.22144, found 587.3 | DMSO-d₆) δ 10.76 (s, |
| | methylmorpholino)ethoxy)phenyl)- | (M + 1)⁺; 585.2 (M − 1)⁻; | 1H), 10.54 (br s, 1H), |
| | 4,5-dimethyl-5- | Retention time: 3.24 | 8.50 (d, J = 5.5 Hz, 1H), |
| | (trifluoromethyl)tetrahydrofuran- | minutes | 8.35 (d, J = 2.2 Hz, 1H), |
| | 2-carboxamido)picolinamide | | 8.06 (d, J = 2.6 Hz, 1H), |
| | (Trifluoroacetate salt) | | 7.80 (dd, J = 5.5, 2.2 Hz, |
| | | | 1H), 7.62 (d, J = 2.9 Hz, |
| | | | 1H), 7.24 (ddd, J = 12.8, |
| | | | 9.5, 6.5 Hz, 2H), 5.16 (d, |
| | | | J = 10.3 Hz, 1H), 4.49 (d, |
| | | | J = 5.5 Hz, 2H), 4.30 (dd, |
| | | | J = 10.3, 7.6 Hz, 1H), |
| | | | 4.04 (d, J = 13.0 Hz, 1H), |
| | | | 3.77 (dd, J = 29.2, 16.6 |
| | | | Hz, 2H), 3.57 (d, J = 38.7 |
| | | | Hz, 4H), 3.16 (s, 1H), |
| | | | 2.90 (s, 1H), 2.78 (p, J = |
| | | | 7.4 Hz, 1H), 1.64 (s, 3H), |
| | | | 1.15 (d, J = 6.2 Hz, 3H), |
| | | | 0.81-0.70 (m, 3H) ppm. |
| 112 | 4-((2R,3S,4S,5R)-3-(2-(2-((2,2- | ESI-MS m/z calc. | ¹H NMR (500 MHz, |
| | difluoroethyl)amino)ethoxy)- | 566.17640, found 567.3 | Methanol-d₄) δ 8.52 (dd, |
| | 3,4-difluorophenyl)-4,5- | (M + 1)⁺; 565.1 (M − 1)⁻; | J = 5.5, 0.6 Hz, 1H), 8.38 |
| | dimethyl-5- | Retention time: 2.41 | (dd, J = 2.2, 0.7 Hz, 1H), |
| | (trifluoromethyl)tetrahydrofuran- | minutes | 7.79 (dd, J = 5.5, 2.2 Hz, |
| | 2-carboxamido)picolinamide | | 1H), 7.23 (ddd, J = 7.7, |
| | (Trifluoroacetate salt) | | 5.5, 2.0 Hz, 1H), 7.14 (td, |
| | | | J = 9.5, 7.7 Hz, 1H), 6.50- |
| | | | 6.25 (m, 1H), 5.11 (d, J = |
| | | | 10.1 Hz, 1H), 4.50- |
| | | | 4.42 (m, 3H), 3.80-3.59 |
| | | | (m, 4H), 2.82 (p, J = 7.7 |
| | | | Hz, 1H), 1.69 (d, J = 1.1 |
| | | | Hz, 3H), 0.89 (dd, J = |
| | | | 7.5, 2.2 Hz, 3H) ppm; |
| | | | amides NH and NH₂ not |
| | | | observed. |
| 113 | 4-((2R,3S,4S,5R)-3-(2-(2-(3- | ESI-MS m/z calc. | ¹H NMR (500 MHz, |
| | oxa-8-azabicyclo[3.2.1]octan-8- | 598.22144, found 599.3 | DMSO-d₆) δ 10.69 (s, |
| | yl)ethoxy)-3,4-difluorophenyl)- | (M + 1)⁺; 597.2 (M − 1)⁻; | 1H), 8.48 (d, J = 5.5 Hz, |
| | 4,5-dimethyl-5- | Retention time: 3.24 | 1H), 8.29 (d, J = 2.1 Hz, |
| | (trifluoromethyl)tetrahydrofuran- | minutes | 1H), 8.07-8.03 (m, 1H), |
| | 2-carboxamido)picolinamide | | 7.83 (dd, J = 5.5, 2.1 Hz, |
| | (Mixture of diastereomers at the | | 1H), 7.60 (s, 1H), 7.21- |
| | 3-oxa-8-azabicyclo[3.2.1]octan- | | 7.10 (m, 2H), 5.13 (d, J = |
| | 8-yl)ethoxy group.) | | 10.2 Hz, 1H), 4.37 (dd, J = |
| | | | 10.3, 7.6 Hz, 1H), 4.21 |
| | | | (ddt, J = 41.0, 11.2, 5.6 |
| | | | Hz, 2H), 3.36 (dd, J = |
| | | | 14.6, 10.1 Hz, 2H), 3.31- |
| | | | 3.23 (m, 4H), 3.02 (d, J = |
| | | | 4.2 Hz, 2H), 2.87 (p, J = |
| | | | 7.5 Hz, 1H), 2.57 (t, J = |
| | | | 5.4 Hz, 2H), 1.77 (td, J = |
| | | | 6.4, 3.2 Hz, 2H), 1.64 (s, |
| | | | 3H), 0.76-0.70 (m, 3H) |
| | | | ppm. |
| 114 | 4-((2R,3S,4S,5R)-3-(3,4- | ESI-MS m/z calc. | ¹H NMR (500 MHz, |
| | difluoro-2-(2- | 516.1796, found 516.7 | DMSO-d₆) δ 10.66 (s, |
| | (methylamino)ethoxy)phenyl)- | (M + 1)⁺; 515.0 (M − 1)⁻; | 1H), 8.49 (d, J = 5.5 Hz, |
| | 4,5-dimethyl-5- | Retention time: 2.90 | 1H), 8.29 (d, J = 2.2 Hz, |
| | (trifluoromethyl)tetrahydrofuran- | minutes | 1H), 8.05 (d, J = 2.8 Hz, |
| | 2-carboxamido)picolinamide | | 1H), 7.83 (dd, J = 5.5, 2.2 |
| | | | Hz, 1H), 7.60 (d, J = 2.8 |
| | | | Hz, 1H), 7.19-7.14 (m, |
| | | | 2H), 5.11 (d, J = 10.6 Hz, |
| | | | 1H), 4.41 (dd, J = 10.6, |
| | | | 7.3 Hz, 1H), 4.19-4.15 |
| | | | (m, 1H), 4.12-4.07 (m, |
| | | | 1H), 2.89-2.83 (m, 1H), |
| | | | 2.83-2.75 (m, 2H), 2.29 |
| | | | (s, 3H), 1.63 (s, 3H), 0.74- |
| | | | 0.68 (m, 3H) ppm; |
| | | | amine NH not observed. |
| 115 | 4-((2R,3S,4S,5R)-3-(3,4- | ESI-MS m/z calc. | ¹H NMR (500 MHz, |
| | difluoro-2-(2- | 544.2109, found 544.8 | DMSO-d₆) δ 10.67 (s, |
| | (isopropylamino)ethoxy)phenyl)- | (M + 1)⁺; 543.0 (M − 1)⁻; | 1H), 8.49 (d, J = 5.5 Hz, |
| | 4,5-dimethyl-5- | Retention time: 3.18 | 1H), 8.30 (d, J = 2.2 Hz, |
| | (trifluoromethyl)tetrahydrofuran- | minutes | 1H), 8.05 (d, J = 2.8 Hz, |
| | 2-carboxamido)picolinamide | | 1H), 7.83 (dd, J = 5.5, 2.2 |
| | | | Hz, 1H), 7.60 (d, J = 2.8 |
| | | | Hz, 1H), 7.21-7.14 (m, |
| | | | 2H), 5.13 (d, J = 10.5 Hz, |
| | | | 1H), 4.35 (dd, J = 10.5, |
| | | | 7.4 Hz, 1H), 4.22-4.13 |
| | | | (m, 2H), 3.31-3.27 (m, |
| | | | 1H), 2.96-2.90 (m, 2H), |
| | | | 2.89-2.82 (m, 1H), 1.63 |
| | | | (s, 3H), 0.98 (s, 6H), 0.76- |
| | | | 0.68 (m, 3H) ppm. |
| 116 | 4-((2R,3S,4S,5R)-3-(3,4- | ESI-MS m/z calc. | ¹H NMR (500 MHz, |
| | difluoro-2-(2-(((R)- | 572.2058, found 573.9 | DMSO-d₆) δ 10.66 (s, |
| | tetrahydrofuran-3- | (M + 1)⁺; 571.0 (M − 1)⁻; | 1H), 8.49 (d, J = 5.5 Hz, |
| | yl)amino)ethoxy)phenyl)-4,5- | Retention time: 2.94 | 1H), 8.29 (d, J = 2.2 Hz, |
| | dimethyl-5- | minutes | 1H), 8.05 (d, J = 2.8 Hz, |
| | (trifluoromethyl)tetrahydrofuran- | | 1H), 7.84 (dd, J = 5.5, 2.2 |
| | 2-carboxamido)picolinamide | | Hz, 1H), 7.60 (d, J = 2.8 |
| | | | Hz, 1H), 7.20-7.13 (m, |
| | | | 2H), 5.12 (d, J = 10.6 Hz, |
| | | | 1H), 4.36 (dd, J = 10.6, |
| | | | 7.4 Hz, 1H), 4.20-4.15 |
| | | | (m, 1H), 4.15-4.10 (m, |
| | | | 1H), 3.71-3.65 (m, 2H), |
| | | | 3.58 (td, J = 8.0, 5.8 Hz, |
| | | | 1H), 3.36 (dd, J = 8.6, 4.4 |
| | | | Hz, 1H), 3.30-3.25 (m, |
| | | | 1H), 2.91-2.79 (m, 3H), |
| | | | 1.97 (s, 1H), 1.91 (ddt, J = |
| | | | 12.3, 7.9, 6.9 Hz, 1H), |
| | | | 1.62 (s, 3H), 1.62-1.57 |
| | | | (m, 1H), 0.71 (d, J = 5.1 |
| | | | Hz, 3H) ppm. |
| 117 | 4-((2R,3S,4S,5R)-3-(3,4- | ESI-MS m/z calc. | ¹H NMR (500 MHz, |
| | difluoro-2-(2-(((S)- | 572.2058, found 573.7 | DMSO-d₆) δ 10.66 (s, |
| | tetrahydrofuran-3- | (M + 1)⁺; 571.0 (M − 1)⁻; | 1H), 8.49 (d, J = 5.5 Hz, |
| | yl)amino)ethoxy)phenyl)-4,5- | Retention time: 2.92 | 1H), 8.30 (d, J = 2.2 Hz, |
| | dimethyl-5- | minutes | 1H), 8.05 (d, J = 2.8 Hz, |
| | (trifluoromethyl)tetrahydrofuran- | | 1H), 7.84 (dd, J = 5.5, 2.2 |
| | 2-carboxamido)picolinamide | | Hz, 1H), 7.60 (d, J = 2.8 |
| | | | Hz, 1H), 7.19-7.14 (m, |
| | | | 2H), 5.12 (d, J = 10.6 Hz, |
| | | | 1H), 4.37 (dd, J = 10.6, |
| | | | 7.4 Hz, 1H), 4.21-4.16 |
| | | | (m, 1H), 4.14-4.09 (m, |
| | | | 1H), 3.73-3.64 (m, 2H), |
| | | | 3.57 (td, J = 8.0, 5.8 Hz, |
| | | | 1H), 3.37 (dd, J = 8.6, 4.4 |
| | | | Hz, 1H), 3.29-3.25 (m, |
| | | | 1H), 2.90-2.80 (m, 3H), |
| | | | 1.99 (s, 1H), 1.91 (ddt, J = |
| | | | 12.3, 7.9, 6.9 Hz, 1H), |
| | | | 1.62 (s, 3H), 1.61-1.56 |
| | | | (m, 1H), 0.71 (d, J = 7.4 |
| | | | Hz, 3H) ppm. |
| 118 | 4-((2R,3S,4S,5R)-3-(3,4- | ESI-MS m/z calc. | ¹H NMR (500 MHz, |
| | difluoro-2-(2-(oxetan-3- | 558.1902, found 558.9 | DMSO-d₆) δ 10.68 (s, |
| | ylamino)ethoxy)phenyl)-4,5- | (M + 1)⁺; 557.0 (M − 1)⁻; | 1H), 8.49 (d, J = 5.5 Hz, |
| | dimethyl-5- | Retention time: 2.83 | 1H), 8.30 (d, J = 2.2 Hz, |
| | (trifluoromethyl)tetrahydrofuran- | minutes | 1H), 8.05 (d, J = 2.8 Hz, |
| | 2-carboxamido)picolinamide | | 1H), 7.84 (dd, J = 5.5, 2.2 |
| | | | Hz, 1H), 7.60 (d, J = 2.8 |
| | | | Hz, 1H), 7.19-7.14 (m, |
| | | | 2H), 5.12 (d, J = 10.6 Hz, |
| | | | 1H), 4.59 (t, J = 6.6 Hz, |
| | | | 2H), 4.38 (dd, J = 10.6, |
| | | | 7.3 Hz, 1H), 4.33-4.30 |
| | | | (m, 2H), 4.17-4.12 (m, |
| | | | 1H), 4.11-4.06 (m, 1H), |
| | | | 3.91 (s, 1H), 2.91-2.78 |
| | | | (m, 3H), 1.62 (s, 3H), |
| | | | 0.71 (d, J = 6.6 Hz, 3H) |
| | | | ppm; amine NH not |
| | | | observed. |
| 119 | 4-((2R,3S,4S,5R)-3-(2-(2-(3- | ESI-MS m/z calc. | ¹H NMR (500 MHz, |
| | (difluoromethoxy)azetidin-1- | 608.18695, found 608.9 | DMSO-d₆) δ 10.69 (s, |
| | yl)ethoxy)-3,4-difluorophenyl)- | (M + 1)⁺; 607.0 (M − 1)⁻; | 1H), 8.49 (d, J = 5.5 Hz, |
| | 4,5-dimethyl-5- | Retention time: 3.33 | 1H), 8.30 (d, J = 2.2 Hz, |
| | (trifluoromethyl)tetrahydrofuran- | minutes | 1H), 8.05 (d, J = 2.8 Hz, |
| | 2-carboxamido)picolinamide | | 1H), 7.86 (dd, J = 5.5, 2.2 |
| | | | Hz, 1H), 7.61 (d, J = 2.8 |
| | | | Hz, 1H), 7.20-7.14 (m, |
| | | | 2H), 6.63 (t, J = 75.2 Hz, |
| | | | 1H), 5.13 (d, J = 10.6 Hz, |
| | | | 1H), 4.70-4.63 (m, 1H), |
| | | | 4.45 (dd, J = 10.6, 7.3 Hz, |
| | | | 1H), 4.18-4.13 (m, 1H), |
| | | | 4.03-3.98 (m, 1H), 3.68- |
| | | | 3.53 (m, 2H), 3.15- |
| | | | 2.95 (m, 2H), 2.89-2.83 |
| | | | (m, 1H), 2.82-2.70 (m, |
| | | | 2H), 1.68 (s, 3H), 0.71 (d, |
| | | | J = 6.1 Hz, 3H) ppm. |
| 120 | 4-((2R,3S,4S,5R)-3-(2-(2-(3- | ESI-MS m/z calc. | ¹H NMR (500 MHz, |
| | (difluoromethyl)azetidin-1- | 592.1921, found 593.0 | DMSO-d₆) δ 10.69 (s, |
| | yl)ethoxy)-3,4-difluorophenyl)- | (M + 1)⁺; 591.0 (M − 1)⁻; | 1H), 8.48 (d, J = 5.5 Hz, |
| | 4,5-dimethyl-5- | Retention time: 3.30 | 1H), 8.28 (d, J = 2.2 Hz, |
| | (trifluoromethyl)tetrahydrofuran- | minutes | 1H), 8.04 (d, J = 2.8 Hz, |
| | 2-carboxamido)picolinamide | | 1H), 7.85 (dd, J = 5.5, 2.2 |
| | | | Hz, 1H), 7.60 (d, J = 2.8 |
| | | | Hz, 1H), 7.18-7.14 (m, |
| | | | 2H), 6.14 (td, J = 56.9, |
| | | | 5.3 Hz, 1H), 5.13 (d, J = |
| | | | 10.6 Hz, 1H), 4.44 (dd, J = |
| | | | 10.6, 7.3 Hz, 1H), 4.17- |
| | | | 4.11 (m, 1H), 3.99- |
| | | | 3.93 (m, 1H), 3.33-3.28 |
| | | | (m, 2H), 3.09 (t, J = 6.9 |
| | | | Hz, 1H), 3.05 (t, J = 6.9 |
| | | | Hz, 1H), 2.88-2.82 (m, |
| | | | 1H), 2.82-2.76 (m, 1H), |
| | | | 2.76-2.71 (m, 1H), 2.71- |
| | | | 2.65 (m, 1H), 1.68 (s, |
| | | | 3H), 0.71 (d, J = 6.9 Hz, |
| | | | 3H) ppm. |
| 121 | 4-((2R,3S,4S,5R)-3-(3,4- | ESI-MS m/z calc. | ¹H NMR (500 MHz, |
| | difluoro-2-(2-(3-fluoro-3- | 574.2015, found 574.8 | DMSO-d₆) δ 10.68 (s, |
| | methylazetidin-1- | (M + 1)⁺; 573.0 (M − 1)⁻; | 1H), 8.49 (d, J = 5.5 Hz, |
| | yl)ethoxy)phenyl)-4,5- | Retention time: 3.32 | 1H), 8.29 (d, J = 2.2 Hz, |
| | dimethyl-5- | minutes | 1H), 8.05 (d, J = 2.8 Hz, |
| | (trifluoromethyl)tetrahydrofuran- | | 1H), 7.84 (dd, J = 5.5, 2.2 |
| | 2-carboxamido)picolinamide | | Hz, 1H), 7.60 (d, J = 2.8 |
| | | | Hz, 1H), 7.19-7.11 (m, |
| | | | 2H), 5.12 (d, J = 10.6 Hz, |
| | | | 1H), 4.38 (dd, J = 10.6, |
| | | | 7.4 Hz, 1H), 4.18-4.12 |
| | | | (m, 1H), 4.06-4.00 (m, |
| | | | 1H), 3.34-3.26 (m, 2H), |
| | | | 3.19 (td, J = 22.4, 8.0 Hz, |
| | | | 2H), 2.90-2.83 (m, 1H), |
| | | | 2.83-2.76 (m, 2H), 1.65 |
| | | | (s, 3H), 1.43 (d, J = 22.4 |
| | | | Hz, 3H), 0.71 (d, J = 6.5 |
| | | | Hz, 3H) ppm. |
| 122 | 4-((2R,3S,4S,5R)-3-(3,4- | ESI-MS m/z calc. | ¹H NMR (500 MHz, |
| | difluoro-2-(2-(3-hydroxy-3- | 572.2058, found 573.0 | DMSO-d₆) δ 10.66 (s, |
| | methylazetidin-1- | (M + 1)⁺; 571.0 (M − 1)⁻; | 1H), 8.49 (d, J = 5.5 Hz, |
| | yl)ethoxy)phenyl)-4,5- | Retention time: 2.84 | 1H), 8.29 (d, J = 2.2 Hz, |
| | dimethyl-5- | minutes | 1H), 8.05 (d, J = 2.8 Hz, |
| | (trifluoromethyl)tetrahydrofuran- | | 1H), 7.85 (dd, J = 5.5, 2.2 |
| | 2-carboxamido)picolinamide | | Hz, 1H), 7.60 (d, J = 2.8 |
| | | | Hz, 1H), 7.18-7.10 (m, |
| | | | 2H), 5.13 (d, J = 10.6 Hz, |
| | | | 1H), 5.06 (s, 1H), 4.37 |
| | | | (dd, J = 10.6, 7.3 Hz, |
| | | | 1H), 4.15-4.10 (m, 1H), |
| | | | 4.05-3.99 (m, 1H), 3.20 |
| | | | (dd, J = 6.7, 1.9 Hz, 1H), |
| | | | 3.17 (dd, J = 6.7, 1.8 Hz, |
| | | | 1H), 2.91-2.84 (m, 2H), |
| | | | 2.81 (d, J = 6.7 Hz, 1H), |
| | | | 2.76-2.69 (m, 2H), 1.65 |
| | | | (s, 3H), 1.24 (s, 3H), 0.71 |
| | | | (d, J = 6.4 Hz, 3H) ppm. |
| 123 | (2R,3S,4S,5R)-3-(2-(2-(6-oxa-2- | ESI-MS m/z calc. | ¹H NMR (500 MHz, |
| | azaspiro[3.4]octan-2- | 694.279, found 695.3 | DMSO-d₆) δ 10.66 (s, |
| | yl)ethoxy)-3,4-difluorophenyl)- | (M + 1)⁺; 693.3 (M − 1)⁻; | 1H), 8.46 (d, J = 5.5 Hz, |
| | N-(2-(6-oxa-2- | Retention time: 3.16 | 1H), 8.21 (d, J = 2.2 Hz, |
| | azaspiro[3.4]octane-2- | minutes | 1H), 7.79 (dd, J = 5.5, 2.2 |
| | carbonyl)pyridin-4-yl)-4,5- | | Hz, 1H), 7.18-7.11 (m, |
| | dimethyl-5- | | 2H), 5.12 (d, J = 10.5 Hz, |
| | (trifluoromethyl)tetrahydrofuran- | | 1H), 4.56-4.50 (m, 2H), |
| | 2-carboxamide | | 4.39 (dd, J = 10.5, 7.4 Hz, |
| | | | 1H), 4.13 (dtd, J = 10.3, |
| | | | 4.1, 1.9 Hz, 1H), 4.03 (s, |
| | | | 2H), 4.00-3.96 (m, 1H), |
| | | | 3.79-3.77 (m, 2H), 3.75- |
| | | | 3.66 (m, 2H), 3.63- |
| | | | 3.58 (m, 2H), 3.57-3.50 |
| | | | (m, 2H), 3.15-3.06 (m, |
| | | | 4H), 2.86 (qd, J = 7.4, 7.4 |
| | | | Hz, 1H), 2.74-2.64 (m, |
| | | | 2H), 2.12 (d, J = 7.0 Hz, |
| | | | 2H), 1.98-1.86 (m, 2H), |
| | | | 1.67 (s, 3H), 0.71 (d, J = |
| | | | 6.7 Hz, 3H) ppm. |
| 124 | 4-((2R,3S,4S,5R)-3-(2-(2-(2- | ESI-MS m/z calc. | ¹H NMR (500 MHz, |
| | oxa-5-azabicyclo[2.2.2]octan-5- | 598.22144, found 599.1 | DMSO-d₆) δ 10.67 (s, |
| | yl)ethoxy)-3,4-difluorophenyl)- | (M + 1)⁺; 597.2 (M − 1)⁻; | 1H), 8.49 (d, J = 5.5 Hz, |
| | 4,5-dimethyl-5- | Retention time: 3.18 | 1H), 8.29 (d, J = 2.2 Hz, |
| | (trifluoromethyl)tetrahydrofuran- | minutes | 1H), 8.05 (d, J = 2.8 Hz, |
| | 2-carboxamido)picolinamide | | 1H), 7.85-7.82 (m, 1H), |
| | (Mixture of diastereomers at the | | 7.60 (d, J = 2.8 Hz, 1H), |
| | 3-oxa-8-azabicyclo[3.2.1]octan- | | 7.19-7.11 (m, 2H), 5.13 |
| | 8-yl)ethoxy group.) | | (d, J = 10.5 Hz, 1H), 4.37- |
| | | | 4.31 (m, 1H), 4.28- |
| | | | 4.21 (m, 1H), 4.19-4.11 |
| | | | (m, 1H), 4.05-3.96 (m, |
| | | | 1H), 3.60-3.57 (m, 1H), |
| | | | 3.56-3.51 (m, 1H), 2.94- |
| | | | 2.80 (m, 5H), 2.60- |
| | | | 2.56 (m, 1H), 1.87-1.72 |
| | | | (m, 2H), 1.63 (s, 3H), |
| | | | 1.56-1.46 (m, 2H), 0.74- |
| | | | 0.69 (m, 3H) ppm. |
| 125 | 4-((2R,3S,4S,5R)-3-(2-(2-(6- | ESI-MS m/z calc. | ¹H NMR (500 MHz, |
| | oxa-1-azaspiro[3.3]heptan-1- | 584.2058, found 585.1 | DMSO-d₆) δ 10.69 (s, |
| | yl)ethoxy)-3,4-difluorophenyl)- | (M + 1)⁺; 583.1 (M − 1)⁻; | 1H), 8.49 (d, J = 5.5 Hz, |
| | 4,5-dimethyl-5- | Retention time: 3.07 | 1H), 8.29 (d, J = 2.2 Hz, |
| | (trifluoromethyl)tetrahydrofuran- | minutes | 1H), 8.05 (d, J = 2.8 Hz, |
| | 2-carboxamido)picolinamide | | 1H), 7.85 (dd, J = 5.5, 2.2 |
| | | | Hz, 1H), 7.60 (d, J = 2.8 |
| | | | Hz, 1H), 7.20-7.13 (m, |
| | | | 2H), 5.14 (d, J = 10.5 Hz, |
| | | | 1H), 4.73 (dd, J = 15.1, |
| | | | 7.3 Hz, 2H), 4.47-4.39 |
| | | | (m, 3H), 4.28-4.22 (m, |
| | | | 1H), 4.09-4.02 (m, 1H), |
| | | | 3.08-3.02 (m, 1H), 3.02- |
| | | | 2.96 (m, 2H), 2.94- |
| | | | 2.89 (m, 1H), 2.85 (p, J = |
| | | | 7.5 Hz, 1H), 2.20 (t, J = |
| | | | 6.9 Hz, 2H), 1.63 (s, 3H), |
| | | | 0.71 (d, J = 7.3 Hz, 3H) |
| | | | ppm. |
| 126 | 4-((2R,3S,4S,5R)-3-(2-(2-(6- | ESI-MS m/z calc. | ¹H NMR (500 MHz, |
| | oxa-2-azaspiro[3.4]octan-2- | 598.22144, found 599.5 | DMSO-d₆) δ 10.67 (s, |
| | yl)ethoxy)-3,4-difluorophenyl)- | (M + 1)⁺; 597.4 (M − 1)⁻; | 1H), 8.48 (d, J = 5.5 Hz, |
| | 4,5-dimethyl-5- | Retention time: 3.12 | 1H), 8.29 (d, J = 2.2 Hz, |
| | (trifluoromethyl)tetrahydrofuran- | minutes | 1H), 8.04 (d, J = 2.9 Hz, |
| | 2-carboxamido)picolinamide | | 1H), 7.84 (dd, J = 5.5, 2.2 |
| | | | Hz, 1H), 7.59 (d, J = 2.9 |
| | | | Hz, 1H), 7.20-7.12 (m, |
| | | | 2H), 5.13 (d, J = 10.6 Hz, |
| | | | 1H), 4.40 (dd, J = 10.6, |
| | | | 7.4 Hz, 1H), 4.16-4.11 |
| | | | (m, 1H), 4.01-3.95 (m, |
| | | | 1H), 3.60 (s, 2H), 3.57- |
| | | | 3.49 (m, 2H), 3.14-3.10 |
| | | | (m, 3H), 3.08 (d, J = 6.8 |
| | | | Hz, 1H), 2.86 (dq, J = |
| | | | 7.4, 7.4 Hz, 1H), 2.74- |
| | | | 2.65 (m, 2H), 1.96-1.86 |
| | | | (m, 2H), 1.67 (s, 3H), |
| | | | 0.71 (d, J = 6.4 Hz, 3H) |
| | | | ppm. |
| 127 | 4-((2R,3S,4S,5R)-3-(2-(2-(2- | ESI-MS m/z calc. | ¹H NMR (500 MHz, |
| | oxa-6-azaspiro[3.4]octan-6- | 598.22144, found 599.1 | DMSO-d₆) δ 10.68 (s, |
| | yl)ethoxy)-3,4-difluorophenyl)- | (M + 1)⁺; 597.2 (M − 1)⁻; | 1H), 8.49 (d, J = 5.5 Hz, |
| | 4,5-dimethyl-5- | Retention time: 3.09 | 1H), 8.30 (d, J = 2.2 Hz, |
| | (trifluoromethyl)tetrahydrofuran- | minutes | 1H), 8.05 (d, J = 2.8 Hz, |
| | 2-carboxamido)picolinamide | | 1H), 7.85 (dd, J = 5.5, 2.2 |
| | | | Hz, 1H), 7.60 (d, J = 2.8 |
| | | | Hz, 1H), 7.18-7.11 (m, |
| | | | 2H), 5.12 (d, J = 10.6 Hz, |
| | | | 1H), 4.40 (d, J = 1.9 Hz, |
| | | | 2H), 4.39 (s, 2H), 4.33 |
| | | | (dd, J = 10.6, 7.4 Hz, |
| | | | 1H), 4.31-4.26 (m, 1H), |
| | | | 4.17-4.12 (m, 1H), 2.83- |
| | | | 2.77 (m, 2H), 2.73- |
| | | | 2.69 (m, 3H), 2.52-2.46 |
| | | | (m, 1H), 2.45-2.40 (m, |
| | | | 1H), 2.01-1.92 (m, 2H), |
| | | | 1.59 (s, 3H), 0.69 (d, J = |
| | | | 6.5 Hz, 3H) ppm. |
| 128 | 4-((2R,3S,4S,5R)-3-(3,4- | ESI-MS m/z calc. | ¹H NMR (500 MHz, |
| | difluoro-2-(2-((R)-3- | 586.22144, found 587.0 | DMSO-d₆) δ 10.63 (s, |
| | methoxypyrrolidin-1- | (M + 1)⁺; 585.2 (M − 1)⁻; | 1H), 8.48 (d, J = 5.5 Hz, |
| | yl)ethoxy)phenyl)-4,5- | Retention time: 3.26 | 1H), 8.32 (d, J = 2.2 Hz, |
| | dimethyl-5- | minutes | 1H), 8.04 (d, J = 2.8 Hz, |
| | (trifluoromethyl)tetrahydrofuran- | | 1H), 7.85 (dd, J = 5.5, 2.2 |
| | 2-carboxamido)picolinamide | | Hz, 1H), 7.59 (d, J = 2.8 |
| | | | Hz, 1H), 7.20-7.11 (m, |
| | | | 2H), 5.13 (d, J = 10.7 Hz, |
| | | | 1H), 4.38 (dd, J = 10.7, |
| | | | 7.4 Hz, 1H), 4.31-4.25 |
| | | | (m, 1H), 4.17-4.12 (m, |
| | | | 1H), 3.79-3.73 (m, 1H), |
| | | | 3.06 (s, 3H), 2.86 (dq, J = |
| | | | 7.4, 7.4 Hz, 1H), 2.74- |
| | | | 2.69 (m, 3H), 2.56-2.44 |
| | | | (m, 2H), 2.39 (dd, J = |
| | | | 10.0, 3.6 Hz, 1H), 1.91- |
| | | | 1.83 (m, 1H), 1.62 (s, |
| | | | 3H), 1.60-1.55 (m, 1H), |
| | | | 0.69 (d, J = 6.4 Hz, 3H) |
| | | | ppm. |
| 129 | 4-((2R,3S,4S,5R)-3-(3,4- | ESI-MS m/z calc. | ¹H NMR (500 MHz, |
| | difluoro-2-(2-((S)-3- | 586.22144, found 587.9 | DMSO-d₆) δ 10.64 (s, |
| | methoxypyrrolidin-1- | (M + 1)⁺; 585.2 (M − 1)⁻; | 1H), 8.49 (d, J = 5.5 Hz, |
| | yl)ethoxy)phenyl)-4,5- | Retention time: 3.24 | 1H), 8.29 (d, J = 2.2 Hz, |
| | dimethyl-5- | minutes | 1H), 8.05 (d, J = 2.8 Hz, |
| | (trifluoromethyl)tetrahydrofuran- | | 1H), 7.85 (dd, J = 5.5, 2.2 |
| | 2-carboxamido)picolinamide | | Hz, 1H), 7.60 (d, J = 2.8 |
| | | | Hz, 1H), 7.19-7.11 (m, |
| | | | 2H), 5.12 (d, J = 10.6 Hz, |
| | | | 1H), 4.35 (dd, J = 10.6, |
| | | | 7.4 Hz, 1H), 4.29-4.24 |
| | | | (m, 1H), 4.19-4.14 (m, |
| | | | 1H), 3.82-3.77 (m, 1H), |
| | | | 3.11 (s, 3H), 2.88 (dq, J = |
| | | | 7.4, 7.4 Hz, 1H), 2.80 |
| | | | (dd, J = 10.0, 6.4 Hz, |
| | | | 1H), 2.77-2.69 (m, 2H), |
| | | | 2.48-2.45 (m, 2H), 2.42 |
| | | | (dd, J = 10.0, 3.8 Hz, |
| | | | 1H), 1.92-1.84 (m, 1H), |
| | | | 1.62 (s, 3H), 1.58-1.52 |
| | | | (m, 1H), 0.73-0.67 (m, |
| | | | 3H) ppm. |

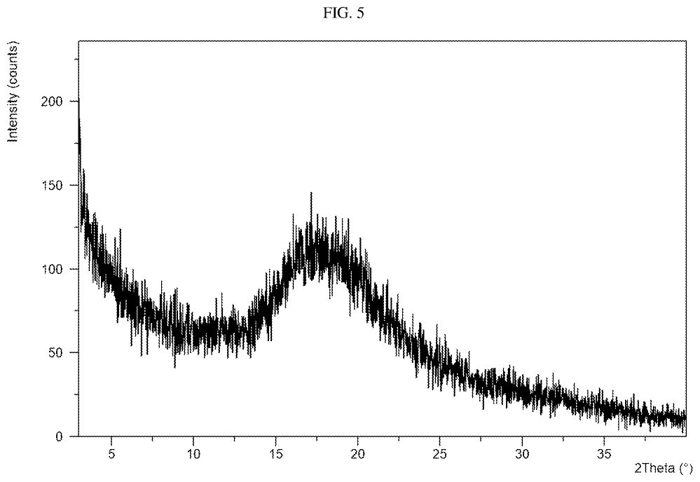

Compound 123 was analyzed by X-ray powder diffraction and determined to be amorphous (see FIG. 5).

The following compound was made using the method described in Example 12, except that the conditions used in step 2 were those described in Example 5 step 2 in the presence of an excess of sodium iodide and using S,S-dimethyl sulfoximine as the amine partner:

| Cmpd No. | Compound Name | LC/MS | NMR (shifts in ppm) |
|---|---|---|---|
| 130 | 4-((2R,3S,4S,5R)-3-(2-(2- | ESI-MS m/z calc. | ¹H NMR (500 MHz, |
| | ((dimethyl(oxo)-26- | 578.16223, found 578.9 | DMSO-d₆) δ 10.72 (s, |
| | sulfaneylidene)amino)ethoxy)- | (M + 1)⁺; 577.0 (M − 1)⁻; | 1H), 8.48 (d, J = 5.6 Hz, |
| | 3,4-difluorophenyl)-4,5- | Retention time: 3.40 | 1H), 8.27 (d, J = 2.2 Hz, |
| | dimethyl-5- | minutes | 1H), 8.05 (d, J = 2.8 Hz, |
| | (trifluoromethyl)tetrahydrofuran- | | 1H), 7.83 (dd, J = 5.5, 2.2 |
| | 2-carboxamido)picolinamide | | Hz, 1H), 7.60 (d, J = 2.8 |
| | | | Hz, 1H), 7.17-7.11 (m, |
| | | | 2H), 5.12 (d, J = 10.4 Hz, |
| | | | 1H), 4.44 (dd, J = 10.4, |
| | | | 7.5 Hz, 1H), 4.25-4.19 |
| | | | (m, 1H), 4.10-4.05 (m, |
| | | | 1H), 3.30-3.26 (m, 2H), |
| | | | 2.99 (s, 3H), 2.97 (s, 3H), |
| | | | 2.93-2.86 (m, 1H), 1.64 |
| | | | (s, 3H), 0.71 (d, J = 5.6 |
| | | | Hz, 3H) ppm. |

The following compound was made using the method described in Example 12, except that the conditions used in step 2 were those described in Example 8 step 5, using 3-oxa-6-azabicyclo[3.1.1]heptane as the amine partner:

| Cmpd No. | Compound Name | LC/MS | NMR (shifts in ppm) |
|---|---|---|---|
| 131 | 4-((2R,3S,4S,5R)-3-(2-(2-(3- | ESI-MS m/z calc. | ¹H NMR (500 MHz, |
| | oxa-6-azabicyclo[3.1.1]heptan- | 584.2058, found 585.3 | DMSO-d₆) δ 10.67 (s, |
| | 6-yl)ethoxy)-3,4- | (M + 1)⁺; 583.2 (M − 1)⁻; | 1H), 8.49 (d, J = 5.5 Hz, |
| | difluorophenyl)-4,5-dimethyl-5- | Retention time: 0.89 | 1H), 8.29 (d, J = 2.2 Hz, |
| | (trifluoromethyl)tetrahydrofuran- | minutes | 1H), 8.05 (d, J = 2.6 Hz, |
| | 2-carboxamido)picolinamide | | 1H), 7.84 (dd, J = 5.5, 2.2 |
| | (Mixture of diastereomers at the | | Hz, 1H), 7.60 (s, 1H), |
| | 3-oxa-8-azabicyclo[3.2.1]octan- | | 7.17 (dd, J = 10.3, 6.6 Hz, |
| | 8-yl)ethoxy group.) | | 2H), 5.14 (d, J = 10.5 Hz, |
| | | | 1H), 4.45 (dd, J = 10.6, |
| | | | 7.4 Hz, 1H), 4.20 (dt, J = |
| | | | 10.0, 5.2 Hz, 1H), 4.13- |
| | | | 4.00 (m, 3H), 3.56 (dd, J = |
| | | | 10.8, 1.6 Hz, 1H), 3.49 |
| | | | (dd, J = 10.8, 1.6 Hz, |
| | | | 1H), 3.45-3.34 (m, 2H), |
| | | | 2.96-2.84 (m, 3H), 2.42 |
| | | | (q, J = 6.8 Hz, 1H), 1.68 |
| | | | (s, 3H), 1.60 (d, J = 8.0 |
| | | | Hz, 1H), 0.76-0.70 (m, |
| | | | 3H) ppm. |

The following compound was made using the method described in Example 12, except that in step 1, 3-bromo-2-(bromomethyl) prop-1-ene was use in place of 1,2-dibromoethane. The reaction conditions used for step 2 were those described in Example 8 step 5, using KOAc in excess and no additional base. The reaction was carried out at ambient temperature:

| Cmpd No. | Compound Name | LC/MS | NMR (shifts in ppm) |
|---|---|---|---|
| 132 | 4-((2R,3S,4S,5R)-3-(3,4- | ESI-MS m/z calc. | ¹H NMR (500 MHz, |
| | difluoro-2-((2- | 529.16364, found 530.2 | DMSO-d₆) δ 10.71 (s, |
| | (hydroxymethyl)allyl)oxy)phenyl)- | (M + 1)⁺; 528.1 (M − 1)⁻; | 1H), 8.50 (d, J = 5.5 Hz, |
| | 4,5-dimethyl-5- | Retention time: 3.01 | 1H), 8.28 (d, J = 2.2 Hz, |
| | (trifluoromethyl)tetrahydrofuran- | minutes | 1H), 8.06 (d, J = 2.6 Hz, |
| | 2-carboxamido)picolinamide | | 1H), 7.83 (dd, J = 5.5, 2.2 |
| | | | Hz, 1H), 7.61 (d, J = 2.9 |
| | | | Hz, 1H), 7.22-7.16 (m, |
| | | | 2H), 5.26-5.20 (m, 2H), |
| | | | 5.12 (d, J = 10.4 Hz, 1H), |
| | | | 4.98 (t, J = 5.5 Hz, 1H), |
| | | | 4.64 (q, J = 11.9 Hz, 2H), |
| | | | 4.29 (dd, J = 10.3, 7.5 Hz, |
| | | | 1H), 4.11-4.05 (m, 2H), |
| | | | 2.76 (p, J = 7.4 Hz, 1H), |
| | | | 1.59 (s, 3H), 0.77-0.71 |
| | | | (m, 3H). |

The following compound was made using the method described in Example 12, except that in step 1, 1,3-dibromopropane was use in place of 1,2-dibromoethane. In step 2, the reaction was carried out at ambient temperature using 3-fluoroazetidine as the amine partner and K₂CO₃ as the base:

| Cmpd No. | Compound Name | LC/MS | NMR (shifts in ppm) |
|---|---|---|---|
| 133 | 4-((2R,3S,4S,5R)-3-(3,4- | ESI-MS m/z calc. | ¹H NMR (500 MHz, |
| | difluoro-2-(3-(3-fluoroazetidin- | 574.2015, found 575.2 | DMSO-d₆) δ 10.72 (s, |
| | 1-yl)propoxy)phenyl)-4,5- | (M + 1)⁺; 573.2 (M − 1)⁻; | 1H), 8.49 (d, J = 5.5 Hz, |
| | dimethyl-5- | Retention time: 3.22 | 1H), 8.29 (d, J = 2.2 Hz, |
| | (trifluoromethyl)tetrahydrofuran- | minutes | 1H), 8.07 (m, 1H)7.83 |
| | 2-carboxamido)picolinamide | | (dd, J = 5.5, 2.2 Hz, 1H), |
| | | | 7.61 (s, 1H), 7.22-7.14 |
| | | | (m, 2H), 5.19-5.07 (m, |
| | | | 2H), 4.30-4.14 (m, 2H), |
| | | | 4.07 (td, J = 7.3, 5.6 Hz, |
| | | | 1H), 3.58-3.47 (m, 2H), |
| | | | 3.08-2.96 (m, 2H), 2.74 |
| | | | (p, J = 7.4 Hz, 1H), 2.59 |
| | | | (t, J = 7.0 Hz, 2H), 1.80- |
| | | | 1.68 (m, 2H), 1.62 (s, |
| | | | 3H), 0.76-0.70 (m, 3H) |
| | | | ppm. |

The following compounds were made using the method described in Example 12, except that in step 1, 1,3-dibromopropane was use in place of 1,2-dibromoethane. In step 2, the reaction was carried out at ambient temperature using azetidine in excess as the amine partner and no base:

| Cmpd No. | Compound Name | LC/MS | NMR (shifts in ppm) |
|---|---|---|---|
| 134 | 4-((2R,3S,4S,5R)-3-(2-(3- | ESI-MS m/z calc. | ¹H NMR (500 MHz, |
| | (azetidin-1-yl)propoxy)-3,4- | 556.21090, found 557.2 | DMSO-d₆) δ 10.71 (s, |
| | difluorophenyl)-4,5-dimethyl-5- | (M + 1)⁺; 555.2 (M − 1)⁻; | 1H), 8.49 (d, J = 5.5 Hz, |
| | (trifluoromethyl)tetrahydrofuran- | Retention time: 3.21 | 1H), 8.29 (d, J = 2.1 Hz, |
| | 2-carboxamido)picolinamide | minutes | 1H), 8.05 (d, J = 2.5 Hz, |
| | | | 1H), 7.84 (dd, J = 5.5, 2.2 |
| | | | Hz, 1H), 7.61 (s, 1H), |
| | | | 7.20-7.14 (m, 2H), 5.11 |
| | | | (d, J = 10.2 Hz, 1H), 4.27 |
| | | | (dd, J = 10.3, 7.6 Hz, |
| | | | 1H), 4.22-4.13 (m, 1H), |
| | | | 4.10-4.02 (m, 1H), 3.02 |
| | | | (t, J = 6.9 Hz, 4H), 2.75 |
| | | | (p, J = 7.5 Hz, 1H), 2.50- |
| | | | 2.43 (m, 2H), 1.88 (p, J = |
| | | | 6.9 Hz, 2H), 1.75-1.65 |
| | | | (m, 2H), 1.63 (s, 3H), |
| | | | 0.76-0.70 (m, 3H) ppm. |

The following compound was made using the method described in Example 12, except that in step 1, 3-bromo-2-(bromomethyl) prop-1-ene was use in place of 1,2-dibromoethane. In step 2, the reaction was carried out at ambient temperature over 16 h using potassium carbonate as the base and excess of MeOH as both the solvent and the alcohol partner:

| Cmpd No. | Compound Name | LC/MS | NMR (shifts in ppm) |
|---|---|---|---|
| 135 | 4-((2R,3S,4S,5R)-3-(3,4- | ESI-MS m/z calc. | ¹H NMR (500 MHz, |
| | difluoro-2-((2- | 543.1793, found 544.1 | Chloroform-d) δ 8.60 (br |
| | (methoxymethyl)allyl)oxy)phenyl)- | (M + 1)⁺; 542.1 (M − 1)⁻; | s, 1H), 8.45 (d, J = 6.0 |
| | 4,5-dimethyl-5- | Retention time: 2.67 | Hz, 1H), 8.14 (dd, J = |
| | (trifluoromethyl)tetrahydrofuran- | minutes | 5.7, 2.1 Hz, 1H), 7.90 (d, |
| | 2-carboxamido)picolinamide | | J = 2.3 Hz, 1H), 7.82 (br |
| | | | s, 1H), 7.11-7.08 (m, 1H), |
| | | | 6.93 (q, J = 8.7 Hz, 1H), |
| | | | 5.54 (s, 1H), 5.28 (s, 1H), |
| | | | 5.22 (s, 1H), 4.99 (d, J = |
| | | | 11.0 Hz, 1H), 4.69 (d, J = |
| | | | 11.9 Hz, 1H), 4.55 (d, J = |
| | | | 11.4 Hz, 1H), 4.14 (dd, J = |
| | | | 11.0, 7.8 Hz, 1H), 4.01 |
| | | | (d, J = 12.8 Hz, 1H), 3.92 |
| | | | (d, J = 12.4 Hz, 1H), 3.21 |
| | | | (s, 3H), 2.75 (t, J = 7.8 |
| | | | Hz, 1H), 1.66 (s, 3H), |
| | | | 0.79 (dd, J = 7.3, 2.3 Hz, |
| | | | 3H) ppm. |

The following compound was made using the method described in Example 12, except that the conditions used in step 2 were those described in Example 5 step 2, using 2-oxa-6-azaspiro[3.3]heptane as the amine partner. In step 3, a methylamine solution (33 wt. % in absolute ethanol) was used in place of methanolic ammonia:

| Cmpd No. | Compound Name | LC/MS | NMR (shifts in ppm) |
|---|---|---|---|
| 136 | 4-((2R,3S,4S,5R)-3-(2-(2-(2- | ESI-MS m/z calc. | ¹H NMR (500 MHz, |
| | oxa-6-azaspiro[3.3]heptan-6- | 598.2215, found 599.28 | Chloroform-d) δ 8.66 (s, |
| | yl)ethoxy)-3,4-difluorophenyl)- | (M + 1)⁺; 597.2 (M − 1)⁻; | 1H), 8.43 (d, J = 5.5 Hz, |
| | 4,5-dimethyl-5- | Retention time: 2.34 | 1H), 8.11 (d, J = 5.5 Hz, |
| | (trifluoromethyl)tetrahydrofuran- | minutes. | 1H), 7.99 (s, 1H), 7.87 (s, |
| | 2-carboxamido)-N- | | 1H), 7.06 (s, 1H), 6.95- |
| | methylpicolinamide | | 6.90 (m, 1H), 5.01 (d, J = |
| | | | 10.5 Hz, 1H), 4.66-4.62 |
| | | | (m, 4H), 4.21-4.04 (m, |
| | | | 3H), 3.33 (d, J = 14.7 Hz, |
| | | | 4H), 3.03 (d, J = 5.0 Hz, |
| | | | 3H), 2.81 (d, J = 8.2 Hz, |
| | | | 1H), 2.72-2.67 (m, 2H), |
| | | | 1.68 (s, 3H), 0.81-0.76 |
| | | | (m, 3H) ppm. |

### Example 13

### rel-4-((2S,3R,4R,5S)-3-(4-fluoro-3-(hydroxymethyl)-2-methoxyphenyl)-4,5-dimethyl-5-(trifluoromethyl)tetrahydrofuran-2-carboxamido) picolinamide (137) and rel-4-((2R,3S,4S,5R)-3-(4-fluoro-3-(hydroxymethyl)-2-methoxyphenyl)-4,5-dimethyl-5-(trifluoromethyl)tetrahydrofuran-2-carboxamido) picolinamide (138)

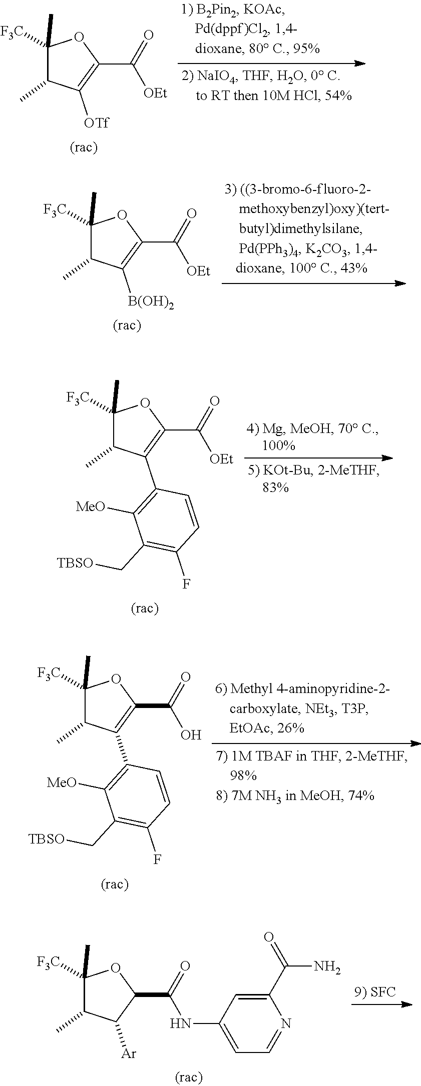

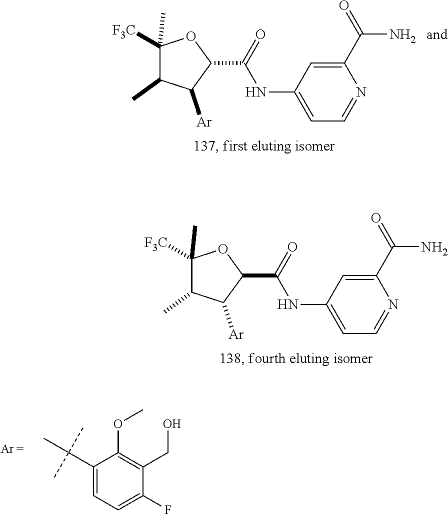

### Step 1 and 2:

To a 3 neck 1 litre flask, flanked with a thermometer and air condenser, was added ethyl rac-(4R,5R)-4,5-dimethyl-5-(trifluoromethyl)-3-(((trifluoromethyl) sulfonyl)oxy)-4,5-dihydrofuran-2-carboxylate (Product of Example 1, Step 4, 42 g, 108.7 mmol) and 1,4-dioxane (500 mL). The mixture was stirred, degassed and flushed with nitrogen. KOAc (32 g, 326.1 mmol) was added followed by bis(pinacolato)diboron (32 g, 126.0 mmol). The reaction mixture was evacuated and back filled with nitrogen (×3). Pd(dppf)Cl₂ (4 g, 5.467 mmol) was added to the reaction mixture, which was then heated to 60° C. first. After stabilizing at 60° C., the temperature was increased to 80° C. (to avoid exotherm). The reaction was allowed to proceed with stirring at 80° C. under nitrogen for 20 h. The reaction mixture was then cooled to ambient temperature and diluted with ethyl acetate (300 mL) and water (100 mL). The mixture was filtered through a pad of celite, and washed several times with ethyl acetate until no more product was eluted from the celite (5×100 ml). The aqueous layer from the filtrates was separated and extracted with ethyl acetate (2×100 mL). The combined organic layers were dried and filtered using Whatman 1PS hydrophobic phase separator filter paper. The filtrates were subsequently concentrated in vacuo to give 47 g of a brown oil. Purification by flash chromatography (Florisil (magnesium silicate) pad, 100% heptane) gave ethyl rac-(4S,5R)-4,5-dimethyl-3-(4,4,5,5-tetramethyl-1,3,2-dioxaborolan-2-yl)-5-(trifluoromethyl)-4,5-dihydrofuran-2-carboxylate (47 g, 95%) as a thick viscous yellow oil. ¹H NMR (500 MHz, Chloroform-d) δ 4.33-4.23 (m, 2H), 3.27-3.18 (m, 1H), 1.55 (d, J=1.1 Hz, 3H), 1.32 (s, 12H), 1.28 (d, J=2.3 Hz, 2H), 1.24 (s, 3H) ppm. ESI-MS m/z calc. 364.1669, found 365.3 (M+1)⁺; Retention time: 1.1 minutes.

NaIO₄ was added (50 g, 233.8 mmol) was added to a solution of ethyl rac-(4S,5R)-4,5-dimethyl-3-(4,4,5,5-tetramethyl-1,3,2-dioxaborolan-2-yl)-5-(trifluoromethyl)-4,5-dihydrofuran-2-carboxylate (47 g) in a 1:2 mixture of water and THF (150 mL). The reaction mixture was stirred for 1 h. The reaction mixture was cooled with an ice bath. 1M HCl (60 mL) was added and reaction mixture was stirred for 60 min. The mixture was then diluted with water (50 mL) and ethyl acetate (100 mL). The resulting white solid was filtered and washed with EtOAc. The filtrate was collected and the phases separated. The organic layer was washed with sodium thiosulphate (3×50 ml), then brine, dried over Na₂SO₄, filtered, and concentrated in vacuo. The cream-colored solid was triturated with cold heptane to give rac-((4S,5R)-2-(ethoxycarbonyl)-4,5-dimethyl-5-(trifluoromethyl)-4,5-dihydrofuran-3-yl) boronic acid (16.657 g, 54%). ¹H NMR (500 MHZ, Chloroform-d) δ 6.84 (s, 2H), 4.38 (q, J=7.1 Hz, 2H), 3.18 (q, J=7.3 Hz, 1H), 1.51 (d, J=1.2 Hz, 3H), 1.39 (t, J=7.1 Hz, 3H), 1.32 (dq, J=7.2, 2.4 Hz, 3H) ppm. ESI-MS m/z calc. 282.08865, found 281.2 (M−1); Retention time: 0.75 minutes.

### Step 3:

Pd(PPh₃)₄ (68 mg, 0.0589 mmol) was added to a mixture of rac-((4S,5R)-2-(ethoxycarbonyl)-4,5-dimethyl-5-(trifluoromethyl)-4,5-dihydrofuran-3-yl) boronic acid (350 mg, 1.241 mmol), ((3-bromo-6-fluoro-2-methoxybenzyl)oxy) (tert-butyl)dimethylsilane (400 mg, 0.996 mmol) and K₂CO₃ (1.9 mL of 2 M, 3.8 mmol) in 1,4-dioxane (10 mL). The reaction mixture was stirred at 100° C. for 5 h. The mixture was then concentrated in vacuo and partitioned between water and EtOAc. The aqueous layer was separated and extracted twice with EtOAc. The combined organic extracts were dried over MgSO₄, filtered, and concentrated in vacuo. Purification by flash chromatography (SiO₂, 0 to 25% EtOAc in heptane) gave ethyl rac-(4S,5R)-3-(3-(((tert-butyldimethylsilyl)oxy)methyl)-4-fluoro-2-methoxyphenyl)-4,5-dimethyl-5-(trifluoromethyl)-4,5-dihydrofuran-2-carboxylate (270 mg, 43%) as a colorless oil. 1H NMR (400 MHZ, Chloroform-d) δ 7.06 (dd, J=8.6, 6.5 Hz, 1H), 6.84 (t, J=8.7 Hz, 1H), 4.73 (dd, J=3.5, 1.8 Hz, 2H), 4.14 (q, J=7.1 Hz, 2H), 3.77 (s, 3H), 3.56 (q, J=7.4 Hz, 1H), 1.70 (d, J=1.0 Hz, 3H), 1.11 (t, J=7.1 Hz, 3H), 1.06 (dq, J=7.2, 2.2 Hz, 3H), 0.90 (s, 9H), 0.11 (d, J=13.6 Hz, 6H) ppm.

### Step 4:

A solution of ethyl rac-(4S,5R)-3-(3-(((tert-butyldimethylsilyl)oxy)methyl)-4-fluoro-2-methoxyphenyl)-4,5-dimethyl-5-(trifluoromethyl)-4,5-dihydrofuran-2-carboxylate (350 mg, 0.691 mmol) in MeOH (12 mL) was added to a two necked flask containing magnesium (196 mg, 8.064 mmol). The reaction was heated at 70° C. for 2 h. A further amount of Mg (60 mg) was added and mixture was stirred at 70° C. for 3 h. The reaction was quenched by addition of a 1 M HCl solution and partitioned between water and EtOAc. The aqueous layer was separated and extracted twice with EtOAc. The combined organic extracts were dried over MgSO₄, filtered and concentrated in vacuo to give methyl rac-(2S,3S,4S,5R)-3-(3-(((tert-butyldimethylsilyl)oxy)methyl)-4-fluoro-2-methoxyphenyl)-4,5-dimethyl-5-(trifluoromethyl)tetrahydrofuran-2-carboxylate (340 mg, 100%) as a mixture of diastereoisomers and as a yellow oil which was used as is in the next step. ESI-MS m/z calc. 494.21115, found 363.2 (M-(TBS+F))⁺; Retention time: 1.28 minutes.

### Step 5:

Potassium tert-butoxide (175 mg, 1.560 mmol) was added to a solution of methyl rac-(2S,3S,4S,5R)-3-(3-(((tert-butyldimethylsilyl)oxy)methyl)-4-fluoro-2-methoxyphenyl)-4,5-dimethyl-5-(trifluoromethyl)tetrahydrofuran-2-carboxylate (260 mg, 0.5257 mmol) in 2-MeTHF (5 mL). The reaction mixture was stirred at ambient temperature for 1 h. The mixture was then quenched by addition of 1 M HCl. The mixture was partitioned between EtOAc and water. The aqueous phase was extracted with EtOAc. The combined organic extracts were washed with brine, dried over MgSO₄, filtered and concentrated in vacuo to give rac-(2R,3S,4S,5R)-3-(3-(((tert-butyldimethylsilyl)oxy)methyl)-4-fluoro-2-methoxyphenyl)-4,5-dimethyl-5-(trifluoromethyl)tetrahydrofuran-2-carboxylic acid (210 mg, 83%) as a yellow oil. ¹H NMR (400 MHZ, Chloroform-d) δ 7.19 (td, J=8.6, 6.2 Hz, 1H), 6.92-6.81 (m, 1H), 4.94 (d, J=10.7 Hz, 1H), 4.72 (d, J=1.8 Hz, 2H), 4.20-4.10 (m, 1H), 3.87 (s, 3H), 2.72 (q, J=7.7 Hz, 1H), 1.64 (d, J=1.1 Hz, 3H), 0.90 (s, 9H), 0.80-0.72 (m, 3H), 0.12 (d, J=8.7 Hz, 6H) ppm; acid OH not observed.

### Step 6:

Et₃N (55 μL, 0.3946 mmol) and T3P (110 μL of 50% w/w, 0.1848 mmol) were successively added to a solution of rac-(2R,3S,4S,5R)-3-(3-(((tert-butyldimethylsilyl)oxy)methyl)-4-fluoro-2-methoxyphenyl)-4,5-dimethyl-5-(trifluoromethyl)tetrahydrofuran-2-carboxylic acid (60 mg, 0.1249 mmol) and methyl 4-aminopyridine-2-carboxylate (32 mg, 0.2103 mmol) in ethyl acetate (1.5 mL). The reaction mixture was stirred overnight at ambient temperature. A further quantity of T3P (50 ul) was added and reaction was stirred at 50° C. for 4 h. The mixture was concentrated in vacuo and loaded onto solid support. Purification by flash chromatography (SiO₂, 0 to 100% EtOAc in heptane) gave methyl rac-4-((2R,3S,4S,5R)-3-(3-(((tert-butyldimethylsilyl)oxy)methyl)-4-fluoro-2-methoxyphenyl)-4,5-dimethyl-5-(trifluoromethyl)tetrahydrofuran-2-carboxamido) picolinate (20 mg, 26%). ¹H NMR (400 MHz, Chloroform-d) δ 8.62 (d, J=5.5 Hz, 1H), 8.58 (s, 1H), 8.10-8.05 (m, 1H), 7.94 (dd, J=5.5, 2.2 Hz, 1H), 7.31 (dd, J=8.8, 6.2 Hz, 1H), 6.91 (t, J=8.7 Hz, 1H), 5.01 (d, J=10.9 Hz, 1H), 4.72 (d, J=1.7 Hz, 2H), 4.18-4.10 (m, 1H), 4.00 (s, 3H), 3.87 (s, 3H), 2.77 (p, J=7.7 Hz, 1H), 1.70 (s, 3H), 0.90 (s, 9H), 0.83-0.75 (m, 3H), 0.13 (s, 6H) ppm.

### Step 7:

TBAF (150 μL of 1 M, 0.1500 mmol) was added to a solution of methyl rac-4-((2R,3S,4S,5R)-3-(3-(((tert-butyldimethylsilyl)oxy)methyl)-4-fluoro-2-methoxyphenyl)-4,5-dimethyl-5-(trifluoromethyl)tetrahydrofuran-2-carboxamido) picolinate (45 mg, 0.07321 mmol) in 2-MeTHF (1 mL). The reaction mixture was stirred at ambient temperature overnight. The mixture was quenched by addition of a saturated NaHCO₃ solution. The mixture was diluted with water and EtOAc. The aqueous layer was separated and extracted twice with EtOAc. The combined organic extracts were dried over MgSO₄, filtered, and concentrated in vacuo to give methyl rac-4-((2R,3S,4S,5R)-3-(4-fluoro-3-(hydroxymethyl)-2-methoxyphenyl)-4,5-dimethyl-5-(trifluoromethyl)tetrahydrofuran-2-carboxamido) picolinate (36 mg, 98%). ESI-MS m/z calc. 500.15704, found 501.2 (M+1)⁺; 499.2 (M−1); Retention time: 0.78 minutes.

### Step 8:

A solution of methyl rac-4-((2R,3S,4S,5R)-3-(4-fluoro-3-(hydroxymethyl)-2-methoxyphenyl)-4,5-dimethyl-5-(trifluoromethyl)tetrahydrofuran-2-carboxamido) picolinate (35 mg, 0.06994 mmol) in methanolic ammonia (1 mL of 7 M, 7.000 mmol) was stirred at ambient temperature for 6 h. A further amount of methanolic ammonia (500 ul of 7M) was added and the reaction was stirred at ambient temperature overnight. The mixture was concentrated in vacuo to give rac-4-((2R,3S,4S,5R)-3-(4-fluoro-3-(hydroxymethyl)-2-methoxyphenyl)-4,5-dimethyl-5-(trifluoromethyl)tetrahydrofuran-2-carboxamido) picolinamide (25 mg, 74%). ESI-MS m/z calc. 485.15738, found 484.2 (M−1); Retention time: 0.73 minutes.

### Step 9:

The stereoisomers of rac-4-((2R,3S,4S,5R)-3-(4-fluoro-3-(hydroxymethyl)-2-methoxyphenyl)-4,5-dimethyl-5-(trifluoromethyl)tetrahydrofuran-2-carboxamido) picolinamide (30 mg, 0.06180 mmol) were separated by chiral SFC using a (R,R)-Whelk-O1column, 5 μm particle size, 25 cm×21.2 mm from Regis Technologies (Mobile phase: 22% methanol (supplemented with 20 mM NH₃), 78% CO₂ for 5 min, then 35% methanol (supplemented with 20 mM NH₃), 65% CO₂ for 2 min; System pressure: 100 bar) on a Prep-100 SFC instrument from Waters. The enantiomers of the major diastereoisomer (first and fourth eluting enantiomers) were collected at this point to give:

First Eluting Isomer (rt=0.65 min): rel-4-((2S,3R,4R,5S)-3-(4-fluoro-3-(hydroxymethyl)-2-methoxyphenyl)-4,5-dimethyl-5-(trifluoromethyl)tetrahydrofuran-2-carboxamido) picolinamide (137, 12 mg, 40%). ¹H NMR (400 MHZ, Methanol-d₄) δ 8.47 (dd, J=5.5, 0.7 Hz, 1H), 8.24 (dd, J=2.2, 0.7 Hz, 1H), 7.88 (dd, J=5.5, 2.2 Hz, 1H), 7.36 (dd, J=8.8, 6.3 Hz, 1H), 6.94 (t, J=8.9 Hz, 1H), 5.05 (d, J=10.5 Hz, 1H), 4.68 (dd, J=1.8, 0.9 Hz, 2H), 4.38 (dd, J=10.6, 8.0 Hz, 1H), 3.89 (s, 3H), 2.79 (p, J=7.6 Hz, 1H), 1.67 (d, J=1.1 Hz, 3H), 0.82 (dt, J=7.5, 2.4 Hz, 3H) ppm; amides NH and NH₂ and alcohol OH not observed. ESI-MS m/z calc. 485.15738, found 486.2 (M+1)⁺; 484.2 (M−1); Retention time: 2.59 minutes.

Fourth Eluting Isomer (rt=1.86 min): rel-4-((2R,3S,4S,5R)-3-(4-fluoro-3-(hydroxymethyl)-2-methoxyphenyl)-4,5-dimethyl-5-(trifluoromethyl)tetrahydrofuran-2-carboxamido) picolinamide (138, 8 mg, 26%). ¹H NMR (400 MHZ, Methanol-d₄) δ 8.47 (dd, J=5.5, 0.6 Hz, 1H), 8.24 (dd, J=2.2, 0.6 Hz, 1H), 7.88 (dd, J=5.5, 2.2 Hz, 1H), 7.36 (dd, J=8.7, 6.3 Hz, 1H), 6.94 (t, J=8.9 Hz, 1H), 5.05 (d, J=10.6 Hz, 1H), 4.68 (dd, J=1.7, 0.9 Hz, 2H), 4.38 (dd, J=10.6, 8.0 Hz, 1H), 3.88 (s, 3H), 2.79 (p, J=7.6 Hz, 1H), 1.67 (d, J=1.2 Hz, 3H), 0.82 (dq, J=7.5, 2.3 Hz, 3H) ppm; amides NH and NH₂ and alcohol OH not observed. ESI-MS m/z calc. 485.15738, found 486.2 (M+1)⁺; 484.2 (M−1); Retention time: 2.59 minutes.

The following compounds were made using the method described in Example 13, except that 1-bromo-4-fluoro-2-methoxy-3-(methoxymethyl)benzene (Intermediate I) was used as the Suzuki coupling partner in step 3. Step 7 was not required. In step 9, purification was performed by chiral SFC using a Chiralpak AS-H column, 5 μm particle size, 25 cm×10 mm from Daicel Corporation (Mobile phase: 28% methanol (supplemented with 20 mM NH₃), 72% CO₂; System pressure: 100 bar) on a Minigram SFC instrument from Berger Instruments:

| Cmpd No. | Compound Name | LC/MS | NMR (shifts in ppm) |
|---|---|---|---|
| 139 | rel-4-((2S,3R,4R,5S)-3-(4- | ESI-MS m/z calc. | ¹H NMR (500 MHz, |
| | fluoro-2-methoxy-3- | 499.17303, found 500.2 | Methanol-d₄) δ 8.48 (dd, |
| | (methoxymethyl)phenyl)-4,5- | (M + 1)⁺; 498.2 (M − 1)⁻; | J = 5.5, 0.6 Hz, 1H), 8.25 |
| | dimethyl-5- | Retention time: 3.04 | (dd, J = 2.2, 0.6 Hz, 1H), |
| | (trifluoromethyl)tetrahydrofuran- | minutes | 7.89 (dd, J = 5.5, 2.2 Hz, |
| | 2-carboxamido)picolinamide | | 1H), 7.39 (dd, J = 8.8, 6.4 |
| | (First eluting isomer by SFC on | | Hz, 1H), 6.96 (t, J = 8.9 |
| | a Chiralpak AS-H column, rt = | | Hz, 1H), 5.06 (d, J = 10.5 |
| | 1.84 min) | | Hz, 1H), 4.60-4.46 (m, |
| | | | 2H), 4.37 (dd, J = 10.5, |
| | | | 8.1 Hz, 1H), 3.86 (s, 3H), |
| | | | 3.39 (d, J = 0.5 Hz, 3H), |
| | | | 2.79 (p, J = 7.7 Hz, 1H), |
| | | | 1.67 (d, J = 1.1 Hz, 3H), |
| | | | 0.81 (dt, J = 7.1, 2.2 Hz, |
| | | | 3H) ppm; amides NH and |
| | | | NH2 not observed. |
| 140 | rel-4-((2R,3S,4S,5R)-3-(4- | ESI-MS m/z calc. | ¹H NMR (500 MHz, |
| | fluoro-2-methoxy-3- | 499.17303, found 500.2 | Methanol-d₄) δ 8.48 (dd, |
| | (methoxymethyl)phenyl)-4,5- | (M + 1)⁺; 498.2 (M − 1)⁻; | J = 5.5, 0.6 Hz, 1H), 8.25 |
| | dimethyl-5- | Retention time: 3.04 | (dd, J = 2.2, 0.7 Hz, 1H), |
| | (trifluoromethyl)tetrahydrofuran- | minutes | 7.89 (dd, J = 5.5, 2.2 Hz, |
| | 2-carboxamido)picolinamide | | 1H), 7.39 (dd, J = 8.8, 6.3 |
| | (Second eluting isomer by SFC | | Hz, 1H), 6.96 (t, J = 8.8 |
| | on a Chiralpak AS-H column, | | Hz, 1H), 5.06 (d, J = 10.5 |
| | rt = 2.73 min) | | Hz, 1H), 4.58-4.46 (m, |
| | | | 2H), 4.37 (dd, J = 10.5, |
| | | | 8.0 Hz, 1H), 3.86 (s, 3H), |
| | | | 3.39 (d, J = 0.5 Hz, 3H), |
| | | | 2.80 (p, J = 7.7 Hz, 1H), |
| | | | 1.67 (d, J = 1.1 Hz, 3H), |
| | | | 0.89-0.69 (m, 3H) ppm; |
| | | | amides NH and NH₂ not |
| | | | observed. |

The following compounds were made using the method described in Example 13, except that the Suzuki step 3 was carried out using the product of step 1. Therefore, step 2 was not required. 1-Bromo-4-(cyclopropylmethoxy)-3-fluoro-2-methoxybenzene (Intermediate K) was used as the coupling partner in the Suzuki step 3. The conditions used in step 4 were those described in Example 14 step 2. The conditions used in step 6 were those described in Example 14 step 5. In step 9, purification was performed by chiral SFC using a (R,R)-Whelk-O1 column, 5 μm particle size, 25 cm×21.2 mm from Regis Technologies (Mobile phase: 70% methanol (supplemented with 20 mM NH₃), 30% CO₂; System pressure: 60 bar) on a Minigram SFC instrument from Berger Instruments:

| Cmpd No. | Compound Name | LC/MS | NMR (shifts in ppm) |
|---|---|---|---|
| 141 | rel-4-((2S,3R,4R,5S)-3-(4- | ESI-MS m/z calc. | ¹H NMR (500 MHz, |
| | (cyclopropylmethoxy)-3-fluoro- | 525.18866, found 526.2 | Chloroform-d) δ 8.73 (s, |
| | 2-methoxyphenyl)-4,5- | (M + 1)⁺; 524.1 (M − 1)⁻; | 1H), 8.50-8.45 (m, 1H), |
| | dimethyl-5- | Retention time: 3.41 | 8.22 (dd, J = 5.7, 2.3 Hz, |
| | (trifluoromethyl)tetrahydrofuran- | minutes | 1H), 8.00 (dd, J = 13.1, |
| | 2-carboxamido)picolinamide | | 5.0 Hz, 2H), 7.07-7.00 |
| | (First eluting isomer by SFC on | | (m, 1H), 6.74-6.66 (m, |
| | a (R,R)-Whelk-O1 column, rt = | | 1H), 5.63 (s, 1H), 5.04 (d, |
| | 2.54 min) | | J = 11.2 Hz, 1H), 4.09 |
| | | | (dd, J = 11.3, 7.9 Hz, |
| | | | 1H), 4.01-3.94 (m, 3H), |
| | | | 3.94-3.87 (m, 2H), 2.76 |
| | | | (p, J = 7.6 Hz, 1H), 1.71 |
| | | | (s, 3H), 1.40-1.30 (m, |
| | | | 1H), 0.82 (dd, J = 7.5, 2.3 |
| | | | Hz, 3H), 0.74-0.66 (m, |
| | | | 2H), 0.44-0.36 (m, 2H) |
| | | | ppm. |
| 142 | rel-4-((2R,3S,4R,5S)-3-(4- | ESI-MS m/z calc. | ¹H NMR (500 MHz, |
| | (cyclopropylmethoxy)-3-fluoro- | 525.18866, found 526.2 | Chloroform-d) δ 8.62 (s, |
| | 2-methoxyphenyl)-4,5- | (M + 1)⁺; 524.1 (M − 1)⁻; | 1H), 8.48 (d, J = 5.7 Hz, |
| | dimethyl-5- | | 1H), 8.14 (dd, J = 5.6, 2.2 |
| | (trifluoromethyl)tetrahydrofuran- | Retention time: 3.34 | Hz, 1H), 8.03-7.88 (m, |
| | 2-carboxamido)picolinamide | minutes | 2H), 6.88 (dd, J = 8.6, 2.0 |
| | (Second eluting isomer by SFC | | Hz, 1H), 6.75-6.64 (m, |
| | on a (R,R)-Whelk-O1 column, | | 1H), 5.59 (s, 1H), 4.79 (d, |
| | rt = 2.89 min) | | J = 9.7 Hz, 1H), 3.98 (d, J = |
| | | | 2.3 Hz, 3H), 3.89 (d, J = |
| | | | 6.9 Hz, 2H), 3.62 (t, J = |
| | | | 11.0 Hz, 1H), 2.56 (d, J = |
| | | | 12.1 Hz, 1H), 1.62 (s, |
| | | | 3H), 1.40-1.28 (m, 1H), |
| | | | 1.07-1.02 (m, 3H), 0.73- |
| | | | 0.65 (m, 2H), 0.39 (dt, J = |
| | | | 6.3, 4.8 Hz, 2H) ppm. |
| 143 | rel-4-((2S,3R,4S,5R)-3-(4- | ESI-MS m/z calc. | ¹H NMR (500 MHz, |
| | (cyclopropylmethoxy)-3-fluoro- | 525.18866, found 526.2 | Chloroform-d) δ 8.62 (s, |
| | 2-methoxyphenyl)-4,5- | (M + 1)⁺; 524.2 (M − 1)⁻; | 1H), 8.48 (d, J = 5.7 Hz, |
| | dimethyl-5- | Retention time: 3.34 | 1H), 8.14 (dd, J = 5.6, 2.2 |
| | (trifluoromethyl)tetrahydrofuran- | minutes | Hz, 1H), 8.03-7.88 (m, |
| | 2-carboxamido)picolinamide | | 2H), 6.88 (dd, J = 8.6, 2.0 |
| | (Third eluting isomer by SFC | | Hz, 1H), 6.75-6.64 (m, |
| | on a (R,R)-Whelk-O1 column, | | 1H), 5.59 (s, 1H), 4.79 (d, |
| | rt = 3.56 min) | | J = 9.7 Hz, 1H), 3.98 (d, J = |
| | | | 2.3 Hz, 3H), 3.89 (d, J = |
| | | | 6.9 Hz, 2H), 3.62 (t, J = |
| | | | 11.0 Hz, 1H), 2.56 (d, J = |
| | | | 12.1 Hz, 1H), 1.62 (s, |
| | | | 3H), 1.40-1.28 (m, 1H), |
| | | | 1.07-1.02 (m, 3H), 0.73- |
| | | | 0.65 (m, 2H), 0.39 (dt, J = |
| | | | 6.3, 4.8 Hz, 2H) ppm. |
| 144 | rel-4-((2R,3S,4S,5R)-3-(4- | ESI-MS m/z calc. | ¹H NMR (500 MHz, |
| | (cyclopropylmethoxy)-3-fluoro- | 525.18866, found 526.2 | Chloroform-d) δ 8.73 (s, |
| | 2-methoxyphenyl)-4,5- | (M + 1)⁺; 524.2 (M − 1)⁻; | 1H), 8.50-8.45 (m, 1H), |
| | dimethyl-5- | Retention time: 3.41 | 8.22 (dd, J = 5.7, 2.3 Hz, |
| | (trifluoromethyl)tetrahydrofuran- | minutes | 1H), 8.00 (dd, J = 13.1, |
| | 2-carboxamido)picolinamide | | 5.0 Hz, 2H), 7.07-7.00 |
| | (Fourth eluting isomer by SFC | | (m, 1H), 6.74-6.66 (m, |
| | on a (R,R)-Whelk-O1 column, | | 1H), 5.63 (s, 1H), 5.04 (d, |
| | rt = 5.72 min) | | J = 11.2 Hz, 1H), 4.09 |
| | | | (dd, J = 11.3, 7.9 Hz, |
| | | | 1H), 4.01-3.94 (m, 3H), |
| | | | 3.94-3.87 (m, 2H), 2.76 |
| | | | (p, J = 7.6 Hz, 1H), 1.71 |
| | | | (s, 3H), 1.40-1.30 (m, |
| | | | 1H), 0.82 (dd, J = 7.5, 2.3 |
| | | | Hz, 3H), 0.74-0.66 (m, |
| | | | 2H), 0.44-0.36 (m, 2H) |
| | | | ppm. |

The following compounds were made using the method described in Example 13, except that the Suzuki coupling step 3 was carried out at 90° C. on the product of step 1 with 1-bromo-3,4-difluoro-2-(methoxymethyl)benzene (Intermediate L) as the aryl bromide in a mixture of 2-MeTHF and water rather than in 1,4-dioxane. The conditions used in the amide coupling step 6 were those described in Example 14 step 5. Step 7 was not required. In step 9, purification was performed by chiral SFC using a (R,R)-Whelk-O1 column, 5 μm particle size, 25 cm×21.1 mm from Daicel Corporation (Mobile phase: 5% to 35% methanol (supplemented with 20 mM NH₃), 95% to 65% CO₂; System pressure: 100 bar) on a Prep-100 SFC instrument from Waters. In the case of compound 145, step 9 was not carried out:

| Cmpd No. | Compound Name | LC/MS | NMR (shifts in ppm) |
|---|---|---|---|
| 145 | rac-4-((2R,3S,4S,5R)-3-(3,4- | ESI-MS m/z calc. | ¹H NMR (500 MHz, |
| | difluoro-2- | 487.153, found 488.1 | Chloroform-d) δ 8.60 (br |
| | (methoxymethyl)phenyl)-4,5- | (M + 1)⁺; 486.1 (M − 1)⁻; | s, 1H), 8.44 (d, J = 5.5 |
| | dimethyl-5- | Retention time: 2.42 | Hz, 1H), 8.12 (dd, J = |
| | (trifluoromethyl)tetrahydrofuran- | minutes | 5.7, 2.1 Hz, 1H), 7.91 (d, |
| | 2-carboxamido)picolinamide | | J = 1.8 Hz, 1H), 7.84 (br |
| | | | s, 1H), 7.21-7.16 (m, 2H), |
| | | | 5.57 (br s, 1H), 5.00 (d, J = |
| | | | 11.0 Hz, 1H), 4.60 (dd, |
| | | | J = 12.1, 1.6 Hz, 1H), |
| | | | 4.49 (dd, J = 12.1, 3.4 Hz, |
| | | | 1H), 4.12 (dd, J = 11.2, |
| | | | 8.5 Hz, 1H), 3.29 (s, 3H), |
| | | | 2.75 (qt, J = 7.8 Hz, 1H), |
| | | | 1.68 (s, 3H), 0.85-0.82 |
| | | | (m, 3H) ppm. |
| 146 | rel-4-((2S,3R,4R,5S)-3-(3,4- | ESI-MS m/z calc. | ¹H NMR (500 MHz, |
| | difluoro-2- | 487.15305, found 488.1 | DMSO-d₆) δ 10.63 (s, |
| | (methoxymethyl)phenyl)-4,5- | (M + 1)⁺; 486.2 (M − 1)⁻; | 1H), 8.47 (d, J = 5.5 Hz, |
| | dimethyl-5- | Retention time: 3.18 | 1H), 8.26 (d, J = 2.2 Hz, |
| | (trifluoromethyl)tetrahydrofuran- | minutes | 1H), 8.04 (d, J = 2.7 Hz, |
| | 2-carboxamido)picolinamide | | 1H), 7.80 (dd, J = 5.5, 2.2 |
| | (First eluting isomer by SFC on | | Hz, 1H), 7.59 (d, J = 2.8 |
| | a (R,R)-Whelk-O1 column, rt = | | Hz, 1H), 7.45 (q, J = 9.1 |
| | 1.38 min) | | Hz, 1H), 7.29 (dd, J = |
| | | | 8.7, 4.5 Hz, 1H), 5.14 (d, |
| | | | J = 10.6 Hz, 1H), 4.54 (d, |
| | | | J = 2.4 Hz, 2H), 4.34 (dd, |
| | | | J = 10.6, 7.6 Hz, 1H), |
| | | | 3.28 (s, 3H), 2.76 (p, J = |
| | | | 7.5 Hz, 1H), 1.63 (s, 3H), |
| | | | 0.73 (d, J = 7.4 Hz, 3H) |
| | | | ppm. |
| 147 | rel-4-((2R,3S,4S,5R)-3-(3,4- | ESI-MS m/z calc. | ¹H NMR (500 MHz, |
| | difluoro-2- | 487.15305, found 488.2 | DMSO-d₆) δ 10.64 (s, |
| | (methoxymethyl)phenyl)-4,5- | (M + 1)⁺; 486.2 (M − 1)⁻; | 1H), 8.47 (d, J = 5.5 Hz, |
| | dimethyl-5- | Retention time: 3.18 | 1H), 8.27 (d, J = 2.1 Hz, |
| | (trifluoromethyl)tetrahydrofuran- | minutes | 1H), 8.04 (d, J = 2.7 Hz, |
| | 2-carboxamido)picolinamide | | 1H), 7.80 (dd, J = 5.5, 2.2 |
| | (Fourth eluting isomer by SFC | | Hz, 1H), 7.59 (d, J = 2.8 |
| | on a (R,R)-Whelk-O1 column, | | Hz, 1H), 7.45 (q, J = 9.1 |
| | rt = 2.34 min) | | Hz, 1H), 7.29 (dd, J = |
| | | | 9.2, 4.5 Hz, 1H), 5.15 (d, |
| | | | J = 10.6 Hz, 1H), 4.54 (d, |
| | | | J = 2.5 Hz, 2H), 4.34 (dd, |
| | | | J = 10.6, 7.6 Hz, 1H), |
| | | | 3.28 (s, 3H), 2.76 (p, J = |
| | | | 7.5 Hz, 1H), 1.63 (s, 3H), |
| | | | 0.75-0.70 (m, 3H) ppm. |

The following compounds were made using the method described in Example 13, except that the Suzuki step 3 was carried out using the product of step 1. Therefore, step 2 was not required. 4-Bromo-1,1,7-trifluoro-2,3-dihydro-1H-indene (Intermediate M) was used as the coupling partner in the Suzuki step 3 and the reaction was carried out at 50° C. for 2 h in the presence of XPhos Pd G4 and K₃PO₄ in a 1:2.5 mixture of water and THF. The conditions used in step 6 were those described in Example 14 step 5. Step 7 was not required. In step 9, purification was performed by chiral SFC using a Chiralcel OJ-H column, 5 μm particle size, 25 cm×10 mm from Daicel Corporation (Mobile phase: 12% methanol (supplemented with 20 mM NH₃), 88% CO₂; System pressure: 100 bar) on a Minigram SFC instrument from Berger Instruments:

| Cmpd No. | Compound Name | LC/MS | NMR (shifts in ppm) |
|---|---|---|---|
| 148 | rel-4-((2S,3R,4R,5S)-4,5- | ESI-MS m/z calc. | ¹H NMR (500 MHz, |
| | dimethyl-3-(1,1,7-trifluoro-2,3- | 501.1487, found 502.1 | DMSO-d₆) δ 10.64 (s, |
| | dihydro-1H-inden-4-yl)-5- | (M + 1)⁺; 500.2 (M − 1)⁻; | 1H), 8.49 (d, J = 5.5 Hz, |
| | (trifluoromethyl)tetrahydrofuran- | Retention time: 3.25 | 1H), 8.30 (d, J = 2.1 Hz, |
| | 2-carboxamido)picolinamide | minutes | 1H), 8.05 (s, 1H), 7.84 |
| | (Second eluting isomer by SFC | | (dd, J = 5.5, 2.2 Hz, 1H), |
| | on a Chiralcel OJ-H column, | | 7.61-7.58 (m, 2H), 7.24 |
| | rt = 5.40 min) | | (t, J = 8.9 Hz, 1H), 5.20 |
| | | | (d, J = 10.3 Hz, 1H), 4.15 |
| | | | (dd, J = 10.2, 7.7 Hz, |
| | | | 1H), 3.21-3.14 (m, 1H), |
| | | | 3.10-3.03 (m, 1H), 2.87 |
| | | | (p, J = 7.5 Hz, 1H), 2.67 |
| | | | (tt, J = 13.9, 6.8 Hz, 2H), |
| | | | 1.64 (s, 3H), 0.71 (d, J = |
| | | | 6.4 Hz, 3H) ppm. |
| 149 | rel-4-((2R,3S,4S,5R)-4,5- | ESI-MS m/z calc. | ¹H NMR (500 MHz, |
| | dimethyl-3-(1,1,7-trifluoro-2,3- | 501.1487, found 502.1 | DMSO-d₆) δ 10.57 (s, |
| | dihydro-1H-inden-4-yl)-5- | (M + 1)⁺; 500.2 (M − 1)⁻; | 1H), 8.42 (d, J = 5.5 Hz, |
| | (trifluoromethyl)tetrahydrofuran- | Retention time: 3.25 | 1H), 8.23 (d, J = 2.1 Hz, |
| | 2-carboxamido)picolinamide | minutes | 1H), 7.98-7.97 (m, 1H), |
| | (Third eluting isomer by SFC | | 7.77 (dd, J = 5.5, 2.1 Hz, |
| | on a Chiralcel OJ-H column, | | 1H), 7.54-7.51 (m, 2H), |
| | rt = 6.01 min) | | 7.17 (t, J = 8.9 Hz, 1H), |
| | | | 5.13 (d, J = 10.3 Hz, 1H), |
| | | | 4.08 (dd, J = 10.1, 7.7 Hz, |
| | | | 1H), 3.14-3.07 (m, 1H), |
| | | | 3.04-2.96 (m, 1H), 2.83- |
| | | | 2.75 (m, 1H), 2.60 (tt, J = |
| | | | 14.1, 6.9 Hz, 2H), 1.57 |
| | | | (s, 3H), 0.64 (d, J = 6.4 |
| | | | Hz, 3H) ppm. |

### Example 14

### rel-4-((2S,3R,5S)-3-(2-chloro-4-(trifluoromethoxy)phenyl)-5-methyl-5-(trifluoromethyl)tetrahydrofuran-2-carboxamido) picolinamide (150) and rel-4-((2R,3S,5R)-3-(2-chloro-4-(trifluoromethoxy)phenyl)-5-methyl-5-(trifluoromethyl)tetrahydrofuran-2-carboxamido) picolinamide (151)

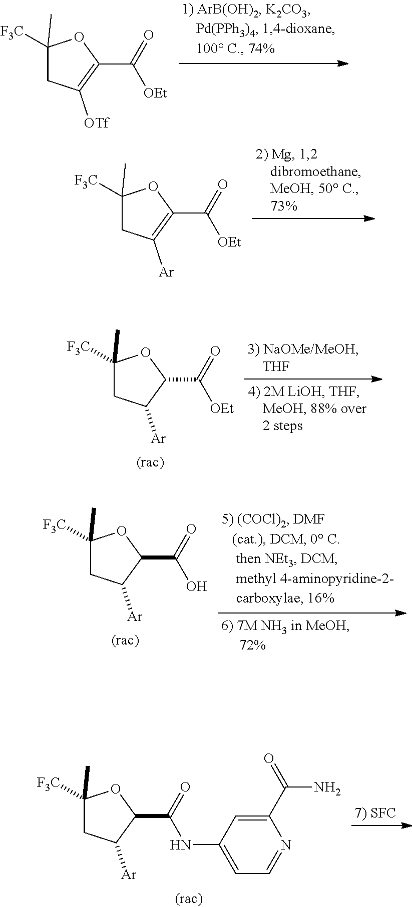

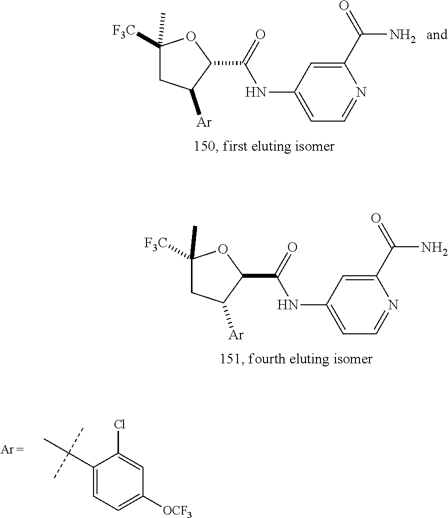

### Step 1:

Pd(PPh₃)₄ (1.3 g, 1.125 mmol) was added to a mixture of (2-chloro-4-(trifluoromethoxy)phenyl) boronic acid (5 g, 20.80 mmol), ethyl rac-5-methyl-5-(trifluoromethyl)-3-(((trifluoromethyl) sulfonyl)oxy)-4,5-dihydrofuran-2-carboxylate (8 g, 21.49 mmol), and sodium carbonate (28.2 mL of 2 M aqueous solution, 56.40 mmol) in 1,4-dioxane (85 mL). The reaction mixture was heated at 100° C. for 3 h. The solution was then partitioned between EtOAc and water. The organic phase was separated and washed with brine. The combined organic extracts were dried over MgSO₄, filtered, and concentrated in vacuo. Purification by flash chromatography (120 g SiO₂, 0 to 40% EtOAc in heptane) gave ethyl rac-3-(2-chloro-4-(trifluoromethoxy)phenyl)-5-methyl-5-(trifluoromethyl)-4,5-dihydrofuran-2-carboxylate (6.46 g, 74%). ¹H NMR (500 MHz, Chloroform-d) δ 7.34 (dd, J=2.3, 1.0 Hz, 1H), 7.32-7.24 (m, 1H), 7.16 (dtd, J=8.5, 2.0, 0.9 Hz, 1H), 4.14 (q, J=7.2 Hz, 2H), 3.43 (d, J=17.5 Hz, 1H), 3.07-2.96 (m, 1H), 1.71 (d, J=1.0 Hz, 3H), 1.08 (t, J=7.1 Hz, 3H) ppm. ESI-MS m/z calc. 418.04065, found 418.8 (M+1)⁺; Retention time: 1.13 minutes.

### Step 2:

A pressure tube was loaded with magnesium powder (2.35 g, 96.69 mmol) and purged with nitrogen. MeOH (20 mL) followed by a solution of ethyl rac-3-(2-chloro-4-(trifluoromethoxy)phenyl)-5-methyl-5-(trifluoromethyl)-4,5-dihydrofuran-2-carboxylate (2 g, 4.777 mmol) in MeOH (20 mL) was added to the reaction vessel. The mixture was degassed with nitrogen. A few drops of 1,2-dibromoethane (80 mg, 0.4258 mmol) were added. The reaction mixture was vigorously stirred and heated at 50° C. for 5 h. The mixture was subsequently cooled to ambient temperature and quenched by pouring it slowly onto a cooled 1 M HCl solution. The resulting mixture was then stirred for 30 min, and subsequently diluted with MTBE. The aqueous layer was separated and extracted with MTBE (×3). The combined organic extracts were passed through a phase separator cartridge. The filtrate was concentrated in vacuo to give methyl rac-(2S,3S,5R)-3-(2-chloro-4-(trifluoromethoxy)phenyl)-5-methyl-5-(trifluoromethyl)tetrahydrofuran-2-carboxylate (2.85 g, 73%) as a mixture of diastereomers, which was used is in the next step without further purification. ESI-MS m/z calc. 406.04065, Retention time: 1.11 minutes; no mass ionisation.

### Step 3 and 4:

Sodium methanolate (310 μL of 25% w/v in methanol, 1.435 mmol) was added to a solution of methyl rac-(2S,3S,5R)-3-(2-chloro-4-(trifluoromethoxy)phenyl)-5-methyl-5-(trifluoromethyl)tetrahydrofuran-2-carboxylate (3.8 g, 9.343 mmol) in THF (40 mL) at ambient temperature under nitrogen. After stirring the mixture for 5 h, methanol (0.2 ml) and LiOH (7.3 mL of 2 M, 14.60 mmol) were added and the reaction mixture was stirred overnight at ambient temperature. The mixture was quenched by adding 1M HCl. The aqueous phase was separated and extracted with MTBE (2×30 ml). The combined organic extracts were washed with brine, dried over Na₂SO₄, filtered, and concentrated in vacuo to give rac-(2R,3S,5R)-3-(2-chloro-4-(trifluoromethoxy)phenyl)-5-methyl-5-(trifluoromethyl)tetrahydrofuran-2-carboxylic acid (3.244 g, 88%) as a mixture of diastereoisomers. ESI-MS m/z calc. 392.025, found 391.0 (M−1); Retention time: 0.63 minutes.

### Step 5:

Oxalyl chloride (117 μL, 1.341 mmol) was carefully added to an ice cold solution of rac-(2R,3S,5R)-3-(2-chloro-4-(trifluoromethoxy)phenyl)-5-methyl-5-(trifluoromethyl)tetrahydrofuran-2-carboxylic acid (250 mg, 0.6367 mmol) and dimethylformamide (0.4 μL, 0.005166 mmol) in dichloromethane (3 mL). The reaction mixture was stirred and warmed up to ambient temperature over 90 min. The mixture was subsequently concentrated in vacuo. The residue was redissolved in dichloromethane (2 mL) and added to an ice cold solution of methyl 4-aminopyridine-2-carboxylate (97 mg, 0.6375 mmol) and triethylamine (266 μL, 1.908 mmol) in dichloromethane (2 mL). The resulting mixture was stirred and warmed to ambient temperature over 18 h. The reaction mixture was then quenched by adding water (5 mL), and the layers were separated. The aqueous layer was extracted with EtOAc (2×10 mL). The combined organic extracts were washed with brine (5 mL), dried over MgSO₄, filtered, and concentrated in vacuo. Purification by flash chromatography (12 g SiO₂, 0 to 50% EtOAc in heptane) gave methyl rac-4-((2R,3S,5R)-3-(2-chloro-4-(trifluoromethoxy)phenyl)-5-methyl-5-(trifluoromethyl)tetrahydrofuran-2-carboxamido) picolinate (55 mg, 16%) as a white solid. ¹H NMR (500 MHz, Chloroform-d) δ 8.56 (d, J=5.5 Hz, 1H), 8.50-8.38 (m, 1H), 8.00 (d, J=2.2 Hz, 1H), 7.87 (dd, J=5.5, 2.2 Hz, 1H), 7.42 (d, J=8.7 Hz, 1H), 7.24 (dd, J=2.5, 1.0 Hz, 1H), 7.14 (ddd, J=8.7, 2.4, 1.1 Hz, 1H), 4.75 (d, J=10.6 Hz, 1H), 4.08-3.98 (m, 1H), 3.93 (s, 3H), 2.47 (dd, J=13.3, 8.2 Hz, 1H), 2.37 (dd, J=13.4, 11.6 Hz, 1H), 1.60 (s, 3H) ppm. ESI-MS m/z calc. 526.073, found 527.2 (M+1)⁺; 525.1 (M−1); Retention time: 1.03 minutes.

### Step 6:

Methanolic ammonia (300 μL of 7 M, 2.100 mmol) was added to a solution of methyl rac-4-((2R,3S,5R)-3-(2-chloro-4-(trifluoromethoxy)phenyl)-5-methyl-5-(trifluoromethyl)tetrahydrofuran-2-carboxamido) picolinate (55 mg, 0.1044 mmol) in methanol (1 mL). The mixture was stirred at ambient temperature for 24 h. The mixture was concentrated in vacuo to give rac-4-((2R,3S,5R)-3-(2-chloro-4-(trifluoromethoxy)phenyl)-5-methyl-5-(trifluoromethyl)tetrahydrofuran-2-carboxamido) picolinamide (49 mg, 72%). ESI-MS m/z calc. 511.07336, found 512.2 (M+1)⁺; 510.1 (M−1); Retention time: 3.39 minutes.

### Step 7:

The enantiomers of rac-4-((2R,3S,5R)-3-(2-chloro-4-(trifluoromethoxy)phenyl)-5-methyl-5-(trifluoromethyl)tetrahydrofuran-2-carboxamido) picolinamide (49 mg, 0.09574 mmol) were separated by chiral SFC using a (R,R)-Whelk-O1column, 5 μm particle size, 25 cm×21.2 mm from Regis Technologies (Mobile phase: 30% acetonitrile: isopropanol (in a 1:1 ratio, supplemented with 0.2% DMPA); System pressure: 100 bar) on a Minigram SFC instrument from Berger Instruments to give:

First Eluting Isomer (rt=2.89 min): rel-4-((2S,3R,5S)-3-(2-chloro-4-(trifluoromethoxy)phenyl)-5-methyl-5-(trifluoromethyl)tetrahydrofuran-2-carboxamido) picolinamide (150, 19 mg, 74%) as a colourless oil. ¹H NMR (400 MHZ, Chloroform-d) δ 8.61 (s, 1H), 8.50 (d, J=5.5 Hz, 1H), 8.19 (dd, J=5.5, 2.3 Hz, 1H), 7.96 (dd, J=2.3, 0.6 Hz, 1H), 7.86 (s, 1H), 7.52 (d, J=8.7 Hz, 1H), 7.33 (dq, J=1.8, 0.9 Hz, 1H), 7.24 (ddd, J=8.7, 2.5, 1.2 Hz, 1H), 5.62 (s, 1H), 4.83 (d, J=10.7 Hz, 1H), 4.11 (td, J=11.2, 8.3 Hz, 1H), 2.61-2.41 (m, 2H), 1.70 (s, 3H) ppm. ESI-MS m/z calc. 511.07336, found 512.1 (M+1)⁺; 510.1 (M−1); Retention time: 3.38 minutes.

Second Eluting Isomer (rt=4.46 min): rel-4-((2R,3S,5R)-3-(2-chloro-4-(trifluoromethoxy)phenyl)-5-methyl-5-(trifluoromethyl)tetrahydrofuran-2-carboxamido) picolinamide (151), which was further purified by column chromatography (4 g SiO₂, 0 to 70% EtOAc in heptane) to give a white solid (18 mg, 72%). ¹H NMR (400 MHZ, Chloroform-d) δ 8.62 (s, 1H), 8.50 (d, J=5.5 Hz, 1H), 8.19 (dd, J=5.5, 2.2 Hz, 1H), 7.97 (d, J=2.2 Hz, 1H), 7.86 (s, 1H), 7.52 (d, J=8.7 Hz, 1H), 7.33 (dd, J=2.5, 1.0 Hz, 1H), 7.24 (ddd, J=8.6, 2.6, 1.2 Hz, 1H), 5.63 (s, 1H), 4.83 (d, J=10.6 Hz, 1H), 4.11 (td, J=11.2, 8.3 Hz, 1H), 2.56 (dd, J=13.2, 8.2 Hz, 1H), 2.47 (dd, J=13.3, 11.6 Hz, 1H), 1.70 (s, 3H) ppm. ESI-MS m/z calc. 511.07336, found 512.1 (M+1)⁺; 510.1 (M−1); Retention time: 3.38 minutes.

### Example 15

### 4-((2R,3S,4S,5R)-3-(3,4-difluoro-2-((R)-2-hydroxypropoxy)phenyl)-4,5-dimethyl-5-(trifluoromethyl)tetrahydrofuran-2-carboxamido) picolinamide (152)

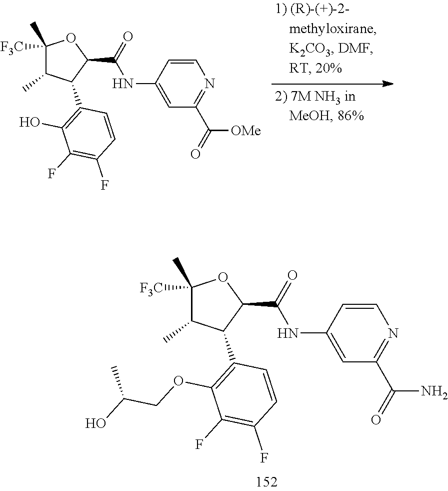 [SMILES: COC(=O)c1cc(NC(=O)[C@@H]2O[C@@](C)(C(F)(F)F)[C@@H](C)[C@H]2c2ccc(F)c(F)c2O)ccn1 ; C[C@@H](O)COc1c([C@H]2[C@H](C(=O)Nc3ccnc(C(N)=O)c3)O[C@@](C)(C(F)(F)F)[C@H]2C)ccc(F)c1F]

### Step 1:

(R)-(+)-2-methyloxirane (116 mg, 140 μL, 1.998 mmol) was added to a solution of methyl 4-((2R,3S,4S,5R)-3-(3,4-difluoro-2-hydroxyphenyl)-4,5-dimethyl-5-(trifluoromethyl)tetrahydrofuran-2-carboxamido) picolinate (Product of Example 8, Step 4.50 mg, 0.101 mmol) and potassium carbonate (18 mg, 0.130 mmol) in DMF (0.5 mL). The reaction mixture was stirred at room temperature for 2 days. Additional (R)-(+)-2-methyloxirane (116 mg, 140 μL, 1.998 mmol) was added and the mixture was stirred at ambient temperature for an additional 3 days. The mixture was heated to 50° C. and stirred for a further 8 h. The mixture was cooled to ambient temperature overnight and then stirred at 80° C. for 8 h. Purification by reverse phase chromatography (12 g SiO₂, 0 to 100% acetonitrile containing 0.1% ammonium hydroxide in water containing 0.1% ammonium hydroxide) gave methyl 4-((2R,3S,4S,5R)-3-(3,4-difluoro-2-((R)-2-hydroxypropoxy)phenyl)-4,5-dimethyl-5-(trifluoromethyl)tetrahydrofuran-2-carboxamido) picolinate (17 mg, 20%) as a white solid. ESI-MS m/z calc. 532.1633, found 533. 6 (M+1)⁺; 531. 4 (M−1); Retention time: 0.95 minutes.

### Step 2:

Methanolic ammonia (1 mL of 7 M, 7.0 mmol) was added to methyl 4-((2R,3S,4S,5R)-3-(3,4-difluoro-2-((R)-2-hydroxypropoxy)phenyl)-4,5-dimethyl-5-(trifluoromethyl)tetrahydrofuran-2-carboxamido) picolinate (17 mg, 0.02 mmol). The reaction mixture was stirred at ambient temperature for 18 h, and then concentrated in vacuo. Purification by reverse phase preparative HPLC using a X-bridge C18 column (150×19 mm, 5 μm particle size) from Waters (Gradient: 20% to 70% acetonitrile in water (supplemented with 0.1% ammonium hydroxide) over 10 min) gave 4-((2R,3S,4S,5R)-3-(3,4-difluoro-2-((R)-2-hydroxypropoxy)phenyl)-4,5-dimethyl-5-(trifluoromethyl)tetrahydrofuran-2-carboxamido) picolinamide (151, 9 mg, 86%). ¹H NMR (500 MHZ, Chloroform-d) δ 8.64 (br s, 1H), 8.46 (d, J=5.5 Hz, 1H), 8.12 (q, J=2.6 Hz, 1H), 7.92 (d, J=1.8 Hz, 1H), 7.85 (br s, 1H), 7.08 (d, J=8.2 Hz, 1H), 6.93 (q, J=8.5 Hz, 1H), 5.55 (br s, 1H), 5.00 (d, J=11.0 Hz, 1H), 4.29 (dd, J=11.0, 7.8 Hz, 1H), 4.21-4.15 (m, 2H), 3.84 (t, J=8.9 Hz, 1H), 2.82-2.74 (m, 1H), 1.67 (s, 3H), 1.20 (d, J=6.4 Hz, 3H), 0.81 (dd, J=7.6, 2.1 Hz, 3H) ppm; OH alcohol not observed. ESI-MS m/z calc. 517.1636, found 518.5 (M+1)⁺; 516.4 (M−1); Retention time: 2.27 minutes.

The following compound was made using the method described in Example 15, except that (S)-2-methyloxirane was used in place of (R)-2-methyloxirane in step 1:

| Cmpd No. | Compound Name | LC/MS | NMR (shifts in ppm) |
|---|---|---|---|
| 153 | 4-((2R,3S,4S,5R)-3-(3,4- | ESI-MS m/z calc. | ¹H NMR (400 MHz, |
| | difluoro-2-((S)-2- | 517.1636, found 518.51 | Chloroform-d) δ 8.66 (br |
| | hydroxypropoxy)phenyl)-4,5- | (M + 1)⁺; 516.4 (M − 1)⁻; | s, 1H), 8.45 (d, J = 5.5 |
| | dimethyl-5- | Retention time: 2.27 | Hz, 1H), 8.12 (dd, J = |
| | (trifluoromethyl)tetrahydrofuran- | minutes | 5.5, 2.3 Hz, 1H), 7.93 (d, |
| | 2-carboxamido)picolinamide | | J = 2.3 Hz, 1H), 7.88 (br |
| | | | s, 1H), 7.09-7.06 (m, 1H), |
| | | | 6.94 (dd, J = 16.7, 9.4 Hz, |
| | | | 1H), 5.57 (br s, 1H), 5.00 |
| | | | (d, J = 11.0 Hz, 1H), 4.35 |
| | | | (dd, J = 11.0, 8.2 Hz, |
| | | | 1H), 4.12-4.03 (m, 3H), |
| | | | 2.83-2.75 (m, 1H), 1.68 |
| | | | (s, 3H), 1.22 (d, J = 6.4 |
| | | | Hz, 3H), 0.81 (dd, J = |
| | | | 7.3, 2.3 Hz, 3H) ppm; |
| | | | alcohol OH not observed. |

### Example 16

### 4-((2R,3S,4S,5R)-3-(5-chloro-3-fluoro-2,4-dimethoxyphenyl)-4,5-dimethyl-5-(trifluoromethyl)tetrahydrofuran-2-carboxamido) picolinamide (154)

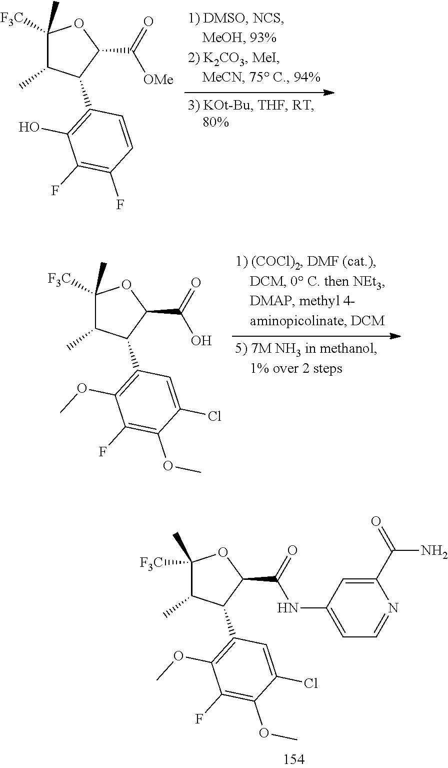 [SMILES: COC(=O)[C@H]1O[C@@](C)(C(F)(F)F)[C@@H](C)[C@H]1c1ccc(F)c(F)c1O ; COc1c(Cl)cc([C@H]2[C@H](C(=O)O)O[C@@](C)(C(F)(F)F)[C@H]2C)c(OC)c1F ; COc1c(Cl)cc([C@H]2[C@H](C(=O)Nc3ccnc(C(N)=O)c3)O[C@@](C)(C(F)(F)F)[C@H]2C)c(OC)c1F]

### Step 1:

NCS (1 g, 7.489 mmol) and DMSO (80 μL, 1.127 mmol) were successively added to a stirred solution of methyl(2S,3S,4S,5R)-3-(3,4-difluoro-2-hydroxyphenyl)-4,5-dimethyl-5-(trifluoromethyl)tetrahydrofuran-2-carboxylate (Product of Example 2, Step 2, 2 g, 5.645 mmol) in MeOH (18 mL) at room temperature and under air. Upon completion of the reaction, the solution was concentrated in vacuo. Purification by flash chromatography gave methyl(2S,3S,4S,5R)-3-(5-chloro-3,4-difluoro-2-hydroxyphenyl)-4,5-dimethyl-5-(trifluoromethyl)tetrahydrofuran-2-carboxylate (2.05 g, 93%). ¹H NMR (500 MHz, Chloroform-d) δ 7.23-7.19 (m, 1H), 5.44 (d, J=4.8 Hz, 1H), 4.81 (d, J=5.9 Hz, 1H), 4.19 (dd, J=8.4, 5.8 Hz, 1H), 3.57 (s, 3H), 2.81-2.71 (m, 1H), 1.46 (d, J=1.3 Hz, 3H), 0.90-0.85 (m, 3H) ppm. ESI-MS m/z calc. 388.05008, found 386.9 (M−1); Retention time: 0.8 minutes.

### Step 2:

In a pressure vial, potassium carbonate (2 g, 14.47 mmol) and iodomethane (650 μL, 10.44 mmol) were added to a mixture of methyl(2S,3S,4S,5R)-3-(5-chloro-3,4-difluoro-2-hydroxyphenyl)-4,5-dimethyl-5-(trifluoromethyl)tetrahydrofuran-2-carboxylate (1.9 g, 4.888 mmol) in MeCN (20 mL). The vial was sealed and heated to 75° C. with stirring for 2 h. Upon completion, the mixture was diluted with a 1:1 mixture of water and brine (20 mL). The aqueous phase was separated and subsequently extracted with DCM. The organic extract was dried over MgSO₄, filtered, and concentrated in vacuo. Purification by flash chromatography gave methyl(2S,3S,4S,5R)-3-(5-chloro-3,4-difluoro-2-methoxyphenyl)-4,5-dimethyl-5-(trifluoromethyl)tetrahydrofuran-2-carboxylate (1.85 g, 94%) as a yellow crystalline solid. 1H NMR (500 MHZ, Chloroform-d) δ 7.30 (ddq, J=7.6, 3.0, 1.6 Hz, 1H), 4.87 (d, J=5.9 Hz, 1H), 4.21 (dd, J=8.6, 6.0 Hz, 1H), 3.94 (d, J=2.1 Hz, 3H), 3.61 (s, 3H), 2.82 (p, J=7.8 Hz, 1H), 1.52 (d, J=1.3 Hz, 3H), 0.87 (dq, J=7.7, 2.0 Hz, 3H) ppm. ESI-MS m/z calc. 402.06573, Retention time: 3.66 minutes; no ionization observed.

### Step 3:

Potassium tert-butoxide (30 mg, 0.2674 mmol) was added to a stirred solution of methyl(2S,3S,4S,5R)-3-(5-chloro-3,4-difluoro-2-methoxyphenyl)-4,5-dimethyl-5-(trifluoromethyl)tetrahydrofuran-2-carboxylate (50 mg, 0.1241 mmol) in THF (500 μL) at ambient temperature. Upon completion of the reaction, the mixture was quenched by addition of a saturated ammonium chloride solution (3 mL) and subsequently diluted with DCM (3 mL). The aqueous phase was separated and extracted with DCM (5 mL). The aqueous phase was then acidified to pH 0 with 1N HCl. The acidic aqueous extracts were further extracted with DCM (2×10 mL). The combined organic extracts were dried over MgSO₄, filtered, and concentrated in vacuo to give (2R,3S,4S,5R)-3-(5-chloro-3-fluoro-2,4-dimethoxyphenyl)-4,5-dimethyl-5-(trifluoromethyl)tetrahydrofuran-2-carboxylic acid (40 mg, 80%), which was used in the next step without further purification. ESI-MS m/z calc. 400.07007, found 399.0 (M−1); Retention time: 0.61 minutes.

### Step 4 and 5:

Oxalyl chloride (123.6 μL, 1.417 mmol) was added to a stirred solution of (2R,3S,4S,5R)-3-(5-chloro-3-fluoro-2,4-dimethoxyphenyl)-4,5-dimethyl-5-(trifluoromethyl)tetrahydrofuran-2-carboxylic acid (250 mg, 0.624 mmol) and DMF (6.183 μL, 0.080 mmol) in DCM (2.5 mL). The reaction mixture was stirred at ambient temperature for 30 min, and then concentrated in vacuo. The residue was dissolved in DCM (1.5 mL) and added to a stirred solution of methyl 4-aminopyridine-2-carboxylate (123.4 mg, 0.811 mmol) and triethylamine (123.6 μL, 0.887 mmol) in DCM (1.5 mL) at ambient temperature. The resulting mixture was stirred for 2 h. The reaction mixture was then quenched by addition of water (1 drop) and MeOH (2 mL) and concentrated in vacuo. Purification by flash chromatography gave methyl 4-((2R,3S,4S,5R)-3-(5-chloro-3-fluoro-2,4-dimethoxyphenyl)-4,5-dimethyl-5-(trifluoromethyl)tetrahydrofuran-2-carboxamido) picolinate, which was immediately used in the next step without further purification. ESI-MS m/z calc. 534.1181, found 535.0 (M+1)⁺; 533.1 (M−1); Retention time: 1.01 minutes.

Methanolic ammonia (20.61 mL of 7 M, 144.3 mmol) was added to methyl 4-((2R,3S,4S,5R)-3-(5-chloro-3-fluoro-2,4-dimethoxyphenyl)-4,5-dimethyl-5-(trifluoromethyl)tetrahydrofuran-2-carboxamido) picolinate and the mixture was stirred overnight at ambient temperature. The mixture was subsequently concentrated in vacuo. Purification by chiral SFC using a Lux i-Cellulose-5 column, 5 μm particle size, 25 cm×10 mm from Phenomenex, Inc. (Mobile phase: 20% methanol (supplemented with 20 mM NH₃), 80% CO₂; System pressure: 100 bar) on a Minigram SFC instrument from Berger Instruments (Retention time: 4.78 minutes) gave 4-((2R,3S,4S,5R)-3-(5-chloro-3-fluoro-2,4-dimethoxyphenyl)-4,5-dimethyl-5-(trifluoromethyl)tetrahydrofuran-2-carboxamido) picolinamide (154, 4.6 mg, 1%). ¹H NMR (500 MHZ, DMSO-d₆) δ 10.66 (s, 1H), 8.50 (d, J=5.5 Hz, 1H), 8.31 (d, J=2.4 Hz, 1H), 8.06 (d, J=2.8 Hz, 1H), 7.85 (dd, J=5.5, 2.2 Hz, 1H), 7.62 (d, J=3.0 Hz, 1H), 7.29 (d, J=2.2 Hz, 1H), 5.13 (d, J=10.1 Hz, 1H), 4.23 (dd, J=10.2, 7.6 Hz, 1H), 3.91 (d, J=1.9 Hz, 3H), 3.90 (d, J=0.9 Hz, 3H), 2.77 (p, J=7.4 Hz, 1H), 1.62 (s, 3H), 0.76 (dd, J=7.6, 2.0 Hz, 3H) ppm. ESI-MS m/z calc. 519.1184, found 520.1 (M+1)⁺; 518.2 (M−1); Retention time: 3.36 minutes.

### Example 17

### 4-((2R,3S,4S,5R)-3-(3,4-difluoro-2-(3-hydroxy-2-(methoxymethyl) propoxy)phenyl)-4,5-dimethyl-5-(trifluoromethyl)tetrahydrofuran-2-carboxamido) picolinamide (155)

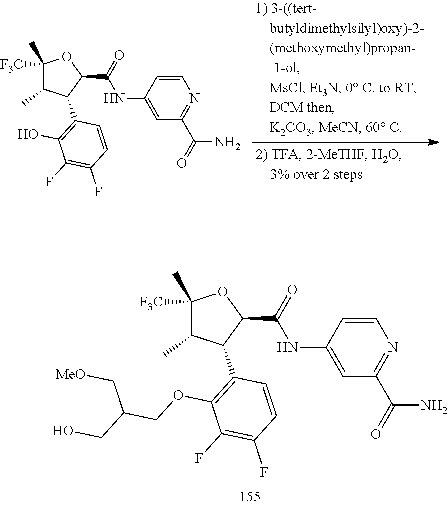 [SMILES: C[C@H]1[C@@H](c2ccc(F)c(F)c2O)[C@H](C(=O)Nc2ccnc(C(N)=O)c2)O[C@@]1(C)C(F)(F)F ; COCC(CO)COc1c([C@H]2[C@H](C(=O)Nc3ccnc(C(N)=O)c3)O[C@@](C)(C(F)(F)F)[C@H]2C)ccc(F)c1F]

### Step 1:

MsCl (25 μL, 0.323 mmol) was added to an ice cold solution of 3-((tert-butyldimethylsilyl)oxy)-2-(methoxymethyl) propan-1-ol (Intermediate U) (60 mg, 0.256 mmol) and Et₃N (55 μL, 0.395 mmol) in DCM (2 mL) under nitrogen. After stirring for 1 h, the ice bath was removed and the solution was stirred at ambient temperature for an additional 3 h. The suspension was partitioned between DCM and water. After stirring for 5 min, the mixture was passed through a phase separation cartridge and then concentrated in vacuo to give 3-((tert-butyldimethylsilyl)oxy)-2-(methoxymethyl) propyl methanesulfonate as a colourless oil, which was used in the next step without further purification.

K₂CO₃ (100 mg, 0.7236 mmol) and 4-((2R,3S,4S,5R)-3-(3,4-difluoro-2-hydroxyphenyl)-4,5-dimethyl-5-(trifluoromethyl)tetrahydrofuran-2-carboxamido) picolinamide (Product of Example 2, Step 10, 50 mg, 0.109 mmol) were successively added to the residue dissolved in MeCN (2 mL) under an atmosphere of nitrogen. The suspension was stirred at 60° C. for 18 h. The reaction mixture was diluted with EtOAc, washed with a saturated sodium bicarbonate aqueous solution and brine. The organic extracts were dried over MgSO₄, filtered, and concentrated in vacuo to give 4-((2R,3S,4S,5R)-3-(2-(3-((tert-butyldimethylsilyl)oxy)-2-(methoxymethyl) propoxy)-3,4-difluorophenyl)-4,5-dimethyl-5-(trifluoromethyl)tetrahydrofuran-2-carboxamido) picolinamide, as a mixture of epimers at the the 2-position of the 3-((tert-butyldimethylsilyl)oxy)-2-(methoxymethyl) propoxy group, which was used in the next step without further purification. ESI-MS m/z calc. 675.2763, found 676.6 (M+1)⁺; 674.5 (M-1); Retention time: 1.25 minutes.

### Step 2:

TFA (250 μL, 3.245 mmol) was added to a solution of 4-((2R,3S,4S,5R)-3-(2-(3-((tert-butyldimethylsilyl)oxy)-2-(methoxymethyl) propoxy)-3,4-difluorophenyl)-4,5-dimethyl-5-(trifluoromethyl)tetrahydrofuran-2-carboxamido) picolinamide in 2-MeTHF (2 mL) and H₂O (100 μL, 5.551 mmol). The reaction mixture was stirred at ambient temperature for 3 h. Purification by reverse phase preparative HPLC gave-4-((2R,3S,4S,5R)-3-(3,4-difluoro-2-(3-hydroxy-2-(methoxymethyl) propoxy)phenyl)-4,5-dimethyl-5-(trifluoromethyl)tetrahydrofuran-2-carboxamido) picolinamide, as a mixture of epimers at the 2-position of the 3-hydroxy-2-(methoxymethyl) propoxy) group (155, 4 mg, 3%). ESI-MS m/z calc. 561.1898, found 562.4 (M+1)⁺; 560.4 (M−1); Retention time: 3.04 minutes.

The following compounds were made using the method described in Example 17, except that different alcohols were used in step 1. Step 2 was not required:

| Cmpd No. | Compound Name | LC/MS | NMR (shifts in ppm) |
|---|---|---|---|
| 156 | 4-((2R,3S,4S,5R)-3-(3,4- | ESI-MS m/z calc. | |
| | difluoro-2-(2-(1- | 543.17926, found 544.4 | |
| | hydroxycyclopropyl)ethoxy)phenyl)- | (M + 1)⁺; 542.4 (M − 1)⁻; | |
| | 4,5-dimethyl-5- | Retention time: 3.17 | |
| | (trifluoromethyl)tetrahydrofuran- | minutes | |
| | 2-carboxamido)picolinamide | | |
| 157 | 4-((2R,3S,4S,5R)-3-(3,4- | ESI-MS m/z calc. | ¹H NMR (500 MHz, |
| | difluoro-2-((R)-1-(oxetan-3- | 543.17926, found 544.4 | Methanol-d₄) δ 8.51 (d, J = |
| | yl)ethoxy)phenyl)-4,5- | (M + 1)⁺; 542.4 (M − 1)⁻; | 5.5 Hz, 1H), 8.27 (d, J = |
| | dimethyl-5- | Retention time: 3.28 | 2.1 Hz, 1H), 7.92 (dd, J = |
| | (trifluoromethyl)tetrahydrofuran- | minutes | 5.5, 2.2 Hz, 1H), 7.17 |
| | 2-carboxamido)picolinamide | | (ddd, J = 8.4, 5.6, 2.1 Hz, |
| | | | 1H), 7.00 (td, J = 9.4, 7.7 |
| | | | Hz, 1H), 5.10 (d, J = 10.7 |
| | | | Hz, 1H),4.83-4.78 |
| | | | (masked, 1H), 4.71 (ddd, |
| | | | J = 8.8, 6.3, 2.7 Hz, 2H), |
| | | | 4.55 (t, J = 6.2 Hz, 1H), |
| | | | 4.49-4.42 (m, 2H), 3.31- |
| | | | 3.25 (m, 1H), 2.79 (p, J = |
| | | | 7.6 Hz, 1H), 1.69 (s, |
| | | | 3H), 1.36 (dd, J = 6.3, 1.0 |
| | | | Hz, 3H), 0.82 (dq, J = |
| | | | 7.4, 2.4 Hz, 3H) ppm; |
| | | | amides NH and NH₂ not |
| | | | observed. |
| 158 | 4-((2R,3S,4S,5R)-3-(3,4- | ESI-MS m/z calc. | ¹H NMR (500 MHz, |
| | difluoro-2-((S)-1-(oxetan-3- | 543.17926, found 544.4 | Methanol-d₄) δ 8.50 (d, J = |
| | yl)ethoxy)phenyl)-4,5- | (M + 1)⁺; 542.4 (M − 1)⁻; | 5.5 Hz, 1H), 8.27 (d, J = |
| | dimethyl-5- | Retention time: 3.15 | 2.1 Hz, 1H), 7.89 (dd, J = |
| | (trifluoromethyl)tetrahydrofuran- | minutes | 5.5, 2.2 Hz, 1H), 7.18 |
| | 2-carboxamido)picolinamide | | (ddd, J = 8.4, 5.6, 2.0 Hz, |
| | | | 1H), 7.04 (td, J = 9.3, 7.6 |
| | | | Hz, 1H), 5.10 (d, J = 10.4 |
| | | | Hz, 1H),4.89-485 |
| | | | (masked, 1H) 4.69 (t, J = |
| | | | 6.2 Hz, 1H), 4.44 (dd, J = |
| | | | 10.4, 7.9 Hz, 1H), 3.39- |
| | | | 3.34 (masked, 4H), 2.83 |
| | | | (p, J = 7.6 Hz, 1H), 1.71- |
| | | | 1.64 (m, 3H), 1.13 (d, J = |
| | | | 6.2 Hz, 3H), 0.81 (dq, J = |
| | | | 7.4, 2.3 Hz, 3H) ppm; |
| | | | amides NH and NH₂ not |
| | | | observed. |
| 159 | 4-((2R,3S,4S,5R)-3-(3,4- | ESI-MS m/z calc. | |
| | difluoro-2-((1- | 529.16364, found 530.4 | |
| | hydroxycyclopropyl)methoxy)phenyl)- | (M + 1)⁺; 528.3 (M − 1)⁻; | |
| | 4,5-dimethyl-5- | Retention time: 3.27 | |
| | (trifluoromethyl)tetrahydrofuran- | minutes | |
| | 2-carboxamido)picolinamide | | |
| 160 | rel-4-((2R*,3S*,4S*,5R*)-3- | ESI-MS m/z calc. | |
| | (3,4-difluoro-2-(3-hydroxy-2- | 547.1742, found 548.5 | |
| | methoxypropoxy)phenyl)-4,5- | (M + 1)⁺; 546.4 (M − 1)⁻; | |
| | dimethyl-5- | Retention time: 2.91 | |
| | (trifluoromethyl)tetrahydrofuran- | minutes | |
| | 2-carboxamido)picolinamide | | |
| 161 | rel-4-((2R*,3S*,4S*,5R*)-3- | ESI-MS m/z calc. | |
| | (3,4-difluoro-2-(3-hydroxy-2- | 547.1742, found 548.5 | |
| | methoxypropoxy)phenyl)-4,5- | (M + 1)⁺; 546.4 (M − 1)⁻; | |
| | dimethyl-5- | Retention time: 2.91 | |
| | (trifluoromethyl)tetrahydrofuran- | minutes | |
| | 2-carboxamido)picolinamide | | |
| 162 | 4-((2R,3S,4S,5R)-3-(3,4- | ESI-MS m/z calc. | ¹H NMR (500 MHz, |
| | difluoro-2-(3- | 543.17926, found 544.7 | Methanol-d₄) δ 8.51 (d, J = |
| | methoxycyclobutoxy)phenyl)- | (M + 1)⁺; 542.6 (M − 1)⁻; | 5.5 Hz, 1H), 8.28 (d, J = |
| | 4,5-dimethyl-5- | Retention time: 3.41 | 2.1 Hz, 1H), 7.91 (dd, J = |
| | (trifluoromethyl)tetrahydrofuran- | minutes | 5.5, 2.2 Hz, 1H), 7.14 |
| | 2-carboxamido)picolinamide | | (ddd, J = 8.0, 5.4, 2.0 Hz, |
| | (Mixture of diastereomers at the | | 1H), 6.99 (td, J = 9.5, 7.6 |
| | 3-methoxycyclobutoxy group.) | | Hz, 1H), 5.09 (d, J = 10.4 |
| | | | Hz, 1H), 4.99 (ddt, J = |
| | | | 7.1, 5.2, 2.7 Hz, 1H), 4.40 |
| | | | (dd, J = 10.4, 7.9 Hz, |
| | | | 1H), 4.15 (tt, J = 6.9, 4.2 |
| | | | Hz, 1H), 3.22 (s, 3H), |
| | | | 2.81 (p, J = 7.7 Hz, 1H), |
| | | | 2.56-2.27 (m, 4H), 1.73- |
| | | | 1.65 (m, 3H), 0.83 (dt, J = |
| | | | 7.5, 2.4 Hz, 3H) ppm; |
| | | | amides NH and NH₂ not |
| | | | observed. |

The following compounds were made using the method described in Example 17, except that different alcohols were used in step 1:

| Cmpd No. | Compound Name | LC/MS | NMR (shifts in ppm) |
|---|---|---|---|
| 163 | 4-((2R,3S,4S,5R)-3-(3,4- | ESI-MS m/z calc. | |
| | difluoro-2-(3- | 529.16364, found 530.4 | |
| | hydroxycyclobutoxy)phenyl)- | (M + 1)⁺; 528.4 (M − 1)⁻; | |
| | 4,5-dimethyl-5- | Retention time: 2.99 | |
| | (trifluoromethyl)tetrahydrofuran- | minutes | |
| | 2-carboxamido)picolinamide | | |
| | (Mixture of diastereomers at the | | |
| | 3-hydroxycyclobutyl group.) | | |
| 164 | rel-4-((2R*,3S*,4S*,5R*)-3- | ESI-MS m/z calc. | |
| | (3,4-difluoro-2-((2- | 543.17926, found 544.6 | |
| | (hydroxymethyl)but-2-en-1- | (M + 1)⁺; 542.6 (M − 1)⁻; | |
| | yl)oxy)phenyl)-4,5-dimethyl-5- | Retention time: 3.08 | |
| | (trifluoromethyl)tetrahydrofuran- | minutes | |
| | 2-carboxamido)picolinamide | | |
| 165 | rel-4-((2R*,3S*,4S*,5R*)-3- | ESI-MS m/z calc. | |
| | (3,4-difluoro-2-((2- | 543.17926, found 544.6 | |
| | (hydroxymethyl)but-2-en-1- | (M + 1)⁺; 542.6 (M − 1)⁻; | |
| | yl)oxy)phenyl)-4,5-dimethyl-5- | Retention time: 3.19 | |
| | (trifluoromethyl)tetrahydrofuran- | minutes | |
| | 2-carboxamido)picolinamide | | |
| 166 | 4-((2R,3S,4S,5R)-3-(3,4- | ESI-MS m/z calc. | ¹H NMR (500 MHz, |
| | difluoro-2-((1s,3R)-3- | 529.16364, found 530.6 | Chloroform-d) δ 8.76 (s, |
| | hydroxycyclobutoxy)phenyl)- | (M + 1)⁺; 528.5 (M − 1)⁻; | 1H), 8.39 (d, J = 5.6 Hz, |
| | 4,5-dimethyl-5- | Retention time: 3.01 | 1H), 8.04 (dd, J = 5.6, 2.2 |
| | (trifluoromethyl)tetrahydrofuran- | minutes | Hz, 1H), 7.97 (d, J = 1.7 |
| | 2-carboxamido)picolinamide | | Hz, 1H), 7.81 (s, 1H), |
| | | | 7.01 (ddd, J = 8.1, 5.4, |
| | | | 2.0 Hz, 1H), 6.82 (td, J = |
| | | | 9.2, 7.4 Hz, 1H), 5.68 (s, |
| | | | 1H), 4.94 (d, J = 10.9 Hz, |
| | | | 1H), 4.34 (pd, J = 6.9, 2.5 |
| | | | Hz, 1H), 4.13 (dd, J = |
| | | | 11.0, 8.0 Hz, 1H), 3.90 |
| | | | (p, J = 6.9 Hz, 1H), 2.85 |
| | | | (dq, J = 12.3, 6.3 Hz, |
| | | | 1H), 2.76-2.67 (m, 2H), |
| | | | 2.17-2.09 (m, 1H), 2.01 |
| | | | (dt, J = 12.3, 7.1 Hz, 1H), |
| | | | 1.63 (s, 3H), 0.73 (dq, J = |
| | | | 7.3, 2.3 Hz, 3H) ppm; OH |
| | | | alcohol not observed. |
| 167 | 4-((2R,3S,4S,5R)-3-(3,4- | ESI-MS m/z calc. | ¹H NMR (500 MHz, |
| | difluoro-2-((1r,3S)-3- | 529.16364, found 530.7 | Methanol-d₄) δ 8.49 (d, J = |
| | hydroxycyclobutoxy)phenyl)- | (M + 1)⁺; 528.6 (M − 1)⁻; | 5.5 Hz, 1H), 8.25 (d, J = |
| | 4,5-dimethyl-5- | Retention time: 3.02 | 2.2 Hz, 1H), 7.90 (dd, J = |
| | (trifluoromethyl)tetrahydrofuran- | minutes | 5.5, 2.2 Hz, 1H), 7.11 |
| | 2-carboxamido)picolinamide | | (ddd, J = 8.0, 5.5, 2.0 Hz, |
| | | | 1H), 6.96 (td, J = 9.5, 7.5 |
| | | | Hz, 1H), 5.10-4.99 (m, |
| | | | 2H), 4.53 (tt, J = 7.1, 4.7 |
| | | | Hz, 1H), 4.41-4.32 (m, |
| | | | 1H), 2.80 (h, J = 7.5, 7.0 |
| | | | Hz, 1H), 2.56 (ddt, J = |
| | | | 11.6, 7.6, 4.1 Hz, 1H), |
| | | | 2.41 (dddt, J = 31.5, 12.0, |
| | | | 7.3, 4.1 Hz, 2H), 2.30- |
| | | | 2.22 (m, 1H), 1.67 (s, |
| | | | 3H), 0.81 (dq, J = 7.4, 2.4 |
| | | | Hz, 3H) ppm; amides NH |
| | | | and NH₂ and alcohol OH |
| | | | not observed. |

The following compound was made using the method described in Example 17, except that different alcohols were used in step 1. The TBMS deprotection step 2 was replaced by a Boc deprotection step, which was carried for 1 h out at ambient temperature using an excess of TFA in DCM, conditions well known in the art:

| | | | |
|---|---|---|---|
| Cmpd No. | Compound Name | LC/MS | NMR (shifts in ppm) |

| | | |
|---|---|---|
| 168 | 4-((2R,3S,4S,5R)-3-(2-((1H- | ESI-MS m/z calc. |
| | pyrazol-4-yl)methoxy)-3,4- | 539.1592, found 540.4 |
| | difluorophenyl)-4,5-dimethyl-5- | (M + 1)⁺; Retention time: |
| | (trifluoromethyl)tetrahydrofuran- | 2.94 minutes |
| | 2-carboxamido)picolinamide | |

The following compound was made using the method described in Example 17, except that in step 1, tosyl chloride was used in place of mesyl chloride together with different alcohols:

| | | | |
|---|---|---|---|
| Cmpd No. | Compound Name | LC/MS | NMR (shifts in ppm) |

| | | |
|---|---|---|
| 169 | 4-((2R,3S,4S,5R)-3-(3,4- | ESI-MS m/z calc. |
| | difluoro-2-(3,3,3-trifluoro-2- | 585.151, found 586.6 |
| | (hydroxymethyl)propoxy)phenyl)- | (M + 1)⁺; 584.6 (M − 1)⁻; |
| | 4,5-dimethyl-5- | Retention time: 3.30 |
| | (trifluoromethyl)tetrahydrofuran- | minutes |
| | 2-carboxamido)picolinamide | |
| | (Mixture of epimers at the | |
| | 3,3,3-trifluoro-2- | |
| | (hydroxymethyl)propoxy | |
| | group.) | |

The following compound was made using the method described in Example 17, except that step 1 was carried out in the presence of an excess of NaI and using different alcohols. Step 2 was not required:

| Cmpd No. | Compound Name | LC/MS | NMR (shifts in ppm) |
|---|---|---|---|
| 170 | 4-((2R,3S,4S,5R)-3-(2-(3- | ESI-MS m/z calc. | ¹H NMR (500 MHz, |
| | acetylcyclobutoxy)-3,4- | 556.1745, found 557.7 | Methanol-d₄) δ 8.49 (d, J = |
| | difluorophenyl)-4,5-dimethyl-5- | (M + 1)⁺; 555.6 (M − 1)⁻; | 5.5 Hz, 1H), 8.30-8.25 |
| | (trifluoromethyl)tetrahydrofuran- | Retention time: 2.87 | (d, J = 2.2 Hz, 1H), 7.91- |
| | 2-carboxamido)picolinamide | minutes | 7.86 (dd, J = 5.5, 2.2 Hz, |
| | (Mixture of diastereomers at the | | 1H), 7.20-7.14 (m, 1H), |
| | 3-acetylcyclobutoxy group.) | | 7.03 (tt, J = 9.5, 7.4 Hz, |
| | | | 1H), 5.17-5.05 (m, 2H), |
| | | | 4.62-4.51 (ddd, J = 10.2, |
| | | | 6.4, 1.6 Hz, 1H), 4.42- |
| | | | 4.29 (m, 3H), 4.10 (ddd, J = |
| | | | 15.0, 11.6, 3.8 Hz, 1H), |
| | | | 2.81 (pd, J = 7.6, 4.3 Hz, |
| | | | 1H), 1.90 (d, J = 10.8 Hz, |
| | | | 3H), 1.66 (d, J = 4.6 Hz, |
| | | | 3H), 0.83 (dq, J = 7.3, 2.4 |
| | | | Hz, 3H) ppm; amides NH |
| | | | and NH2 not observed. |

The following compounds were made using the method described in Example 17, except that different alcohols were used in step 1 and DMF was used as the solvent in place of MeCN. Step 2 was not required. In the case of compound 172, rac-(1s,3s)-1-(trifluoromethyl)cyclobutane-1,3-diol was used as the alcohol in step 1 and compound 172 was isolated as the sole product of reaction:

| Cmpd No. | Compound Name | LC/MS | NMR (shifts in ppm) |
|---|---|---|---|
| 171 | 4-((2R,3S,4S,5R)-3-(2-((1,1- | ESI-MS m/z calc. | ¹H NMR (500 MHz, |
| | dioxidothietan-3-yl)oxy)-3,4- | 563.1149, found 564.6 | Methanol-d₄) δ 8.52 (d, |
| | difluorophenyl)-4,5-dimethyl-5- | (M + 1)⁺; 562.6 (M − 1)⁻; | 1H), 8.30 (d, 1H), 7.93 |
| | (trifluoromethyl)tetrahydrofuran- | Retention time: 2.86 | (d, 1H), 7.09-7.04 (m, |
| | 2-carboxamido)picolinamide | minutes | 1H), 6.77-6.69 (m, 1H), |
| | (Trifluoroacetate salt) | | 5.13-5.11 (d, 1H), 4.78- |
| | | | 4.75 (m, 1H), 4.59-4.53 |
| | | | (m, 2H), 4.38-4.32 (m, |
| | | | 3H), 2.94-2.87 (m, 1H), |
| | | | 1.68 (s, 3H), 0.84-0.82 |
| | | | (m, 3H) ppm; amides NH |
| | | | and NH₂, H salt not |
| | | | observed. |
| 172 | 4-((2R,3S,4S,5R)-3-(3,4- | ESI-MS m/z calc. | ¹H NMR (500 MHz, |
| | difluoro-2-(((E)-3- | 579.14044, found 580.6 | DMSO-d₆) δ 10.67 (s, |
| | (trifluoromethyl)buta-1,3-dien- | (M + 1)⁺; 578.6 (M − 1)⁻; | 1H), 8.49-8.48 (d, 1H), |
| | 1-yl)oxy)phenyl)-4,5-dimethyl- | Retention time: 3.7 | 8.27-8.26 (d, 1H), 8.05 |
| | 5- | minutes | (d, 1H), 7.83-7.82 (dd, |
| | (trifluoromethyl)tetrahydrofuran- | | 1H), 7.60 (d, 1H), 7.43- |
| | 2-carboxamido)picolinamide | | 7.38 (m, 1H), 7.32-7.26 |
| | | | (m, 2H), 5.88-5.86 (d, |
| | | | 1H), 5.72 (s, 1H), 5.63 (s, |
| | | | 1H), 5.17-5.14 (d, 1H), |
| | | | 4.19-4.15 (m, 1H), 2.75- |
| | | | 2.72 (m, 1H), 1.58-1.57 |
| | | | (d, 3H), 0.78-0.77 (d, 3H) |
| | | | ppm. |
| 173 | 4-((2R,3S,4S,5R)-3-(2-(3,3- | ESI-MS m/z calc. | ¹H NMR (500 MHz, |
| | difluorocyclobutoxy)-3,4- | 549.14984, found 550.2 | Methanol-d₄) δ 8.49 (d, J = |
| | difluorophenyl)-4,5-dimethyl-5- | (M + 1)⁺; 548.2 (M − 1)⁻; | 5.5 Hz, 1H), 8.26 (d, J = |
| | (trifluoromethyl)tetrahydrofuran- | Retention time: 3.55 | 2.2 Hz, 1H), 7.90 (dd, J = |
| | 2-carboxamido)picolinamide | minutes | 5.5, 2.2 Hz, 1H), 7.16 |
| | | | (ddd, J = 8.2, 5.5, 2.1 Hz, |
| | | | 1H), 7.02 (td, J = 9.4, 7.6 |
| | | | Hz, 1H), 5.09 (d, J = 10.5 |
| | | | Hz, 1H), 4.89 (dtd, J = |
| | | | 7.6, 4.8, 2.3 Hz, 1H), 4.37 |
| | | | (dd, J = 10.5, 7.9 Hz, |
| | | | 1H), 3.19-2.93 (m, 2H), |
| | | | 2.92-2.72 (m, 3H), 1.67 |
| | | | (s, 3H), 0.82 (dq, J = 7.4, |
| | | | 2.3 Hz, 3H) ppm; amides |
| | | | NH and NH₂ not |
| | | | observed. |
| 174 | 4-((2R,3S,4S,5R)-3-(3,4- | ESI-MS m/z calc. | ¹H NMR (500 MHz, |
| | difluoro-2-(3-hydroxy-3- | 543.17926, found 544.3 | Methanol-d₄) δ 8.49 (d, J = |
| | methylcyclobutoxy)phenyl)- | (M + 1)⁺; 542.3 (M − 1)⁻; | 5.5 Hz, 1H), 8.25 (d, J = |
| | 4,5-dimethyl-5- | Retention time: 3.13 | 2.2 Hz, 1H), 7.90 (dd, J = |
| | (trifluoromethyl)tetrahydrofuran- | minutes | 5.5, 2.3 Hz, 1H), 7.11 |
| | 2-carboxamido)picolinamide | | (t, J = 7.2 Hz, 1H), 6.96 |
| | (Mixture of diastereomers at the | | (q, J = 8.8 Hz, 1H), 5.07 |
| | 3-hydroxy-3- | | (d, J = 10.5 Hz, 1H), 4.97- |
| | methylcyclobutoxy group.) | | 4.91 (m, 1H), 4.37 (dd, J = |
| | | | 10.5, 7.9 Hz, 1H), 2.78 |
| | | | (t, J = 7.7 Hz, 1H), 2.57- |
| | | | 2.50 (m, 1H), 2.43 (dt, J = |
| | | | 12.3, 6.4 Hz, 1H), 2.31 |
| | | | (dd, J = 12.8, 5.8 Hz, |
| | | | 1H), 2.24-2.16 (m, 1H), |
| | | | 1.67 (s, 3H), 1.40 (s, 3H), |
| | | | 0.85-0.76 (m, 3H) ppm; |
| | | | amides NH and NH₂ and |
| | | | alcohol OH not observed. |

The following compound was made using the method described in Example 17, except that different alcohols were used in step 1 and DMF was used as the solvent in place of MeCN:

| | | | |
|---|---|---|---|
| Cmpd No. | Compound Name | LC/MS | NMR (shifts in ppm) |

| | | |
|---|---|---|
| 175 | 4-((2R,3S,4S,5R)-3-(3,4- | ESI-MS m/z calc. |
| | difluoro-2-((3- | 543.17926, found 544.2 |
| | hydroxycyclopentyl)oxy) | (M + 1)⁺; 542.3 (M − 1)⁻; |
| | phenyl)-4,5-dimethyl-5- | Retention time: 3.04 |
| | (trifluoromethyl)tetrahydrofuran- | minutes |
| | 2-carboxamido)picolinamide | |
| | (Mixture of diastereomers | |
| | having (1S,3R) or (1R,3S) | |
| | stereochemical configuration at | |
| | the 3-hydroxycyclopentyl | |
| | group.) | |

The following compound was made using the method described in Example 17, except that in step 1, the reaction was carried out at 100° C. for 18h, using 4-hydroxy-1-methylpyrrolidin-2-one as the alcohol, Cs₂CO₃ as the base, a catalytic amount of LiBr as an additive and DMF as the solvent. Step 2 was not required. Compound 176 was isolated as a side product and as the sole product of reaction:

| Cmpd No. | Compound Name | LC/MS | NMR (shifts in ppm) |
|---|---|---|---|
| 176 | 4-((2R,3S,4S,5R)-3-(5-bromo- | ESI-MS m/z calc. | ¹H NMR (500 MHz, |
| | 3,4-difluoro-2-hydroxyphenyl)- | 537.0323, found 540.5 | Methanol-d₄) δ 8.51 (d, |
| | 4,5-dimethyl-5- | (M + 1)⁺; 536.4 (M − 1)⁻; | 1H), 8.29 (d, 1H), 7.94- |
| | (trifluoromethyl)tetrahydrofuran- | Retention time: 2.88 | 7.92 (dd, 1H), 7.30-7.28 |
| | 2-carboxamido)picolinamide | minutes | (dd, 1H), 5.12-5.10 (d, |
| | | | 1H), 4.36-4.32 (dd, 1H), |
| | | | 2.95-2.92 (t, 1H), 1.67 (s, |
| | | | 3H), 0.86-0.85 (m, 3H) |
| | | | ppm; amides NH and |
| | | | NH₂ and alcohol OH not |
| | | | observed. |

### Example 18

### 4-((2R,3S,4S,5R)-3-(5-(benzyloxy)-3,4-difluoro-2-methoxyphenyl)-4,5-dimethyl-5-(trifluoromethyl)tetrahydrofuran-2-carboxamido) picolinamide (177), 4-((2R,3S,4S,5R)-3-(3,4-difluoro-5-hydroxy-2-methoxyphenyl)-4,5-dimethyl-5-(trifluoromethyl)tetrahydrofuran-2-carboxamido) picolinamide (178) and 4-((2R,3S,4S,5R)-3-(5-(3,3-difluorocyclobutoxy)-3,4-difluoro-2-methoxyphenyl)-4,5-dimethyl-5-(trifluoromethyl)tetrahydrofuran-2-carboxamido) picolinamide (179)

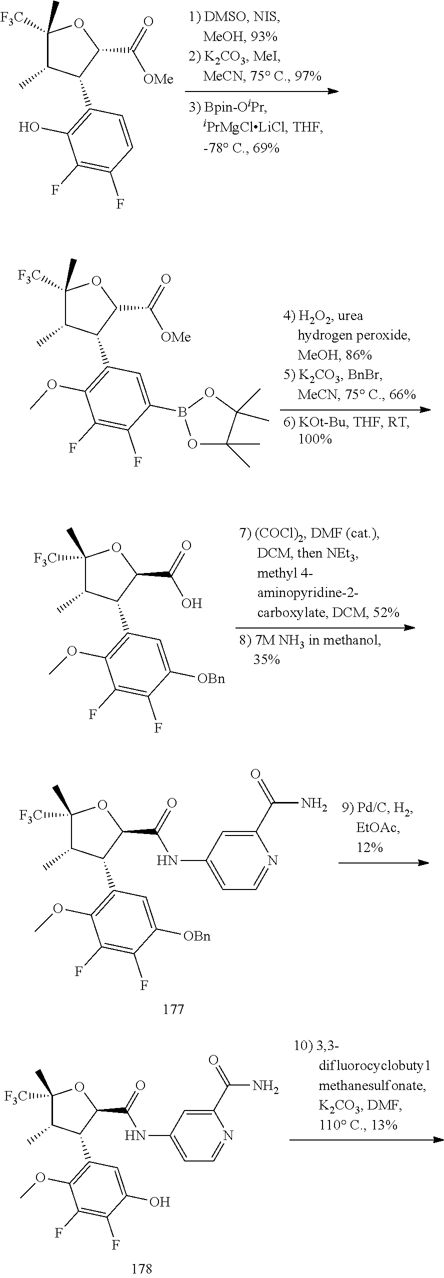

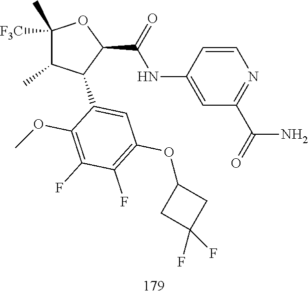 [SMILES: COc1c([C@H]2[C@H](C(=O)Nc3ccnc(C(N)=O)c3)O[C@@](C)(C(F)(F)F)[C@H]2C)cc(OC2CC(F)(F)C2)c(F)c1F]

### Step 1:

DMSO (80 μL, 1.127 mmol) and NIS (1.7 g, 7.556 mmol) were sequentially added to a stirred solution of methyl(2S,3S,4S,5R)-3-(3,4-difluoro-2-hydroxyphenyl)-4,5-dimethyl-5-(trifluoromethyl)tetrahydrofuran-2-carboxylate (Product of Example 2, Step 2, 2.2 g, 6.210 mmol) in MeOH (20 mL) at ambient temperature. The reaction mixture was stirred at ambient temperature under air for 30 min. Upon completion of the reaction, the mixture was concentrated in vacuo. Purification by flash chromatography gave methyl(2S,3S,4S,5R)-3-(3,4-difluoro-2-hydroxy-5-iodophenyl)-4,5-dimethyl-5-(trifluoromethyl)tetrahydrofuran-2-carboxylate (2.78 g, 93%). ¹H NMR (500 MHZ, Chloroform-d) δ 7.49 (dt, J=6.4, 2.1 Hz, 1H), 5.56 (d, J=4.9 Hz, 1H), 4.81 (d, J=5.9 Hz, 1H), 4.16 (dd, J=8.3, 5.9 Hz, 1H), 3.60 (s, 3H), 2.75 (p, J=7.7 Hz, 1H), 1.45 (d, J=1.2 Hz, 3H), 0.90-0.85 (m, 3H) ppm. ESI-MS m/z calc. 479.9857, found 481.1 (M+1)⁺; 479.1 (M−1); Retention time: 0.8 minutes.

### Step 2:

K₂CO₃ (2.5 g, 18.09 mmol) and Mel (1 mL, 16.06 mmol) were successively added to a solution of methyl(2S,3S,4S,5R)-3-(3,4-difluoro-2-hydroxy-5-iodophenyl)-4,5-dimethyl-5-(trifluoromethyl)tetrahydrofuran-2-carboxylate (2.8 g, 5.831 mmol) in MeCN (25 mL). The reaction mixture was heated to 75° C. in a sealed vial for 90 min. Upon completion of the reaction, the mixture was partitioned between DCM and a saturated aqueous NaCl solution. The organic phase was separated, dried over MgSO₄, filtered, and concentrated in vacuo to give methyl(2S,3S,4S,5R)-3-(3,4-difluoro-5-iodo-2-methoxyphenyl)-4,5-dimethyl-5-(trifluoromethyl)tetrahydrofuran-2-carboxylate (2.8 g, 97%). ¹H NMR (500 MHZ, Chloroform-d) δ 7.53 (dq, J=6.5, 1.5 Hz, 1H), 4.80 (d, J=6.1 Hz, 1H), 4.11 (dd, J=8.5, 5.8 Hz, 1H), 3.88 (d, J=2.4 Hz, 3H), 3.56 (s, 3H), 2.73 (p, J=8.4, 7.8 Hz, 1H), 1.45 (d, J=1.1 Hz, 3H), 0.80 (dd, J=7.4, 1.9 Hz, 3H) ppm. ESI-MS m/z calc. 494.00134, found 495.2 (M+1)⁺; Retention time: 1.06 minutes.

### Step 3:

ⁱPrMgCl·LiCl (1.2 mL of 1.3 M in THF, 1.560 mmol) was added dropwise to a stirred solution of methyl(2S,3S,4S,5R)-3-(3,4-difluoro-5-iodo-2-methoxyphenyl)-4,5-dimethyl-5-(trifluoromethyl)tetrahydrofuran-2-carboxylate (700 mg, 1.416 mmol) in THF (6 mL) at −78° C. The resulting solution was stirred for 15 min at −78° C. 2-Isopropoxy-4,4,5,5-tetramethyl-1,3,2-dioxaborolane (350 μL, 1.716 mmol) was subsequently added and the reaction mixture was allowed to warm to ambient temperature. The reaction was quenched by addition of a saturated ammonium chloride solution and then extracted with DCM. The organic extracts were dried over MgSO₄, filtered and concentrated in vacuo. Purification by flash chromatography (12 g SiO₂, 0 to 100% AcOEt in heptane) gave methyl(2S,3S,4S,5R)-3-(3,4-difluoro-2-methoxy-5-(4,4,5,5-tetramethyl-1,3,2-dioxaborolan-2-yl)phenyl)-4,5-dimethyl-5-(trifluoromethyl)tetrahydrofuran-2-carboxylate (480 mg, 69%), which was used in the next step without further purification. ESI-MS m/z calc. 494.1899, found 495.5 (M+1)⁺; Retention time: 1.09 minutes.

### Step 4:

Urea hydrogen peroxide complex (150 mg, 1.595 mmol) was added in one portion to a stirred solution of methyl(2S,3S,4S,5R)-3-(3,4-difluoro-2-methoxy-5-(4,4,5,5-tetramethyl-1,3,2-dioxaborolan-2-yl)phenyl)-4,5-dimethyl-5-(trifluoromethyl)tetrahydrofuran-2-carboxylate (600 mg, 1.214 mmol) in MeOH (2.5 mL). The solution was stirred overnight at ambient temperature. The mixture was then concentrated in vacuo. Purification by flash chromatography gave methyl(2S,3S,4S,5R)-3-(3,4-difluoro-5-hydroxy-2-methoxyphenyl)-4,5-dimethyl-5-(trifluoromethyl)tetrahydrofuran-2-carboxylate (400 mg, 86%) as a white solid. ESI-MS m/z calc. 384.0996, found 383.3 (M−1); Retention time: 0.85 minutes.

### Step 5:

K₂CO₃ (250 mg, 1.809 mmol) and benzyl bromide (200 μL, 1.682 mmol) were successively added to a solution of methyl(2S,3S,4S,5R)-3-(3,4-difluoro-5-hydroxy-2-methoxyphenyl)-4,5-dimethyl-5-(trifluoromethyl)tetrahydrofuran-2-carboxylate (210 mg, 0.5465 mmol) in MeCN (2 mL). The reaction mixture was heated to 75° C. for 90 min in a sealed vial. The mixture was subsequently partitioned between DCM (10 mL) and a saturated NaCl solution (20 mL). The organic phase was separated, dried over MgSO₄, filtered, and concentrated in vacuo. Purification by flash chromatography (12 g SiO₂, 0-100% EtOAc in heptane) gave methyl(2S,3S,4S,5R)-3-(5-(benzyloxy)-3,4-difluoro-2-methoxyphenyl)-4,5-dimethyl-5-(trifluoromethyl)tetrahydrofuran-2-carboxylate (170 mg, 66%), which was used as is in the next step. ESI-MS m/z calc. 474.14658, Retention time: 1.11 minutes; (no ionization observed).

### Step 6:

Potassium tert-butoxide (160 mg, 1.426 mmol) was added to a stirred solution of methyl(2S,3S,4S,5R)-3-(5-(benzyloxy)-3,4-difluoro-2-methoxyphenyl)-4,5-dimethyl-5-(trifluoromethyl)tetrahydrofuran-2-carboxylate (170 mg, 0.3583 mmol) in THF (3 mL) at ambient temperature. The reaction mixture was stirred for 5 min at ambient temperature. The mixture was subsequently quenched by addition of a saturated ammonium chloride solution (3 mL) and diluted with DCM (3 mL). The aqueous phase was separated and extracted with DCM (5 mL). The aqueous phase was acidified to pH 0 with 1N HCl. The aqueous extracts were further extracted with DCM (2×10 mL). The combined extracts were dried over MgSO₄, filtered, and concentrated in vacuo to give (2R,3S,4S,5R)-3-(5-(benzyloxy)-3,4-difluoro-2-methoxyphenyl)-4,5-dimethyl-5-(trifluoromethyl)tetrahydrofuran-2-carboxylic acid (165 mg, 100%), which was used in the next step without further purification. ESI-MS m/z calc. 460.13092, found 459.5 (M−1); Retention time: 0.7 minutes.

### Step 7:

Oxalyl chloride (75 μL, 0.860 mmol) was added dropwise to a stirred solution of (2R,3S,4S,5R)-3-(5-(benzyloxy)-3,4-difluoro-2-methoxyphenyl)-4,5-dimethyl-5-(trifluoromethyl)tetrahydrofuran-2-carboxylic acid (165 mg, 0.358 mmol) and DMF (4 μL, 0.052 mmol) in DCM (1.5 mL) at ambient temperature. The reaction mixture was stirred at ambient temperature for 30 min, and the solution was subsequently concentrated in vacuo. The residue was dissolved in DCM (1 mL) and added into a stirred solution of methyl 4-aminopyridine-2-carboxylate (75 mg, 0.493 mmol) and TEA (75 μL, 0.538 mmol) in DCM (1 mL) at ambient temperature. The reaction mixture was stirred for 2 h at ambient temperature. The mixture was quenched by addition of methanol (100 μL) and concentrated in vacuo. Purification by flash chromatography gave methyl 4-((2R,3S,4S,5R)-3-(5-(benzyloxy)-3,4-difluoro-2-methoxyphenyl)-4,5-dimethyl-5-(trifluoromethyl)tetrahydrofuran-2-carboxamido) picolinate (110 mg, 52%), which was immediately taken as is in the next step. ESI-MS m/z calc. 594.17896, found 595.7 (M+1)⁺; 593.6 (M−1); Retention time: 1.08 minutes.

### Step 8:

Methanolic ammonia (12 mL of 7 M, 84.00 mmol) was added to methyl 4-((2R,3S,4S,5R)-3-(5-(benzyloxy)-3,4-difluoro-2-methoxyphenyl)-4,5-dimethyl-5-(trifluoromethyl)tetrahydrofuran-2-carboxamido) picolinate (110 mg). The reaction mixture was stirred at ambient temperature until complete conversion was observed. Purification by flash chromatography gave 4-((2R,3S,4S,5R)-3-(5-(benzyloxy)-3,4-difluoro-2-methoxyphenyl)-4,5-dimethyl-5-(trifluoromethyl)tetrahydrofuran-2-carboxamido) picolinamide (177, 80 mg, 35%). ¹H NMR (500 MHZ, Chloroform-d) δ 8.59 (s, 1H), 8.48 (d, J=5.6 Hz, 1H), 8.15 (dd, J=5.6, 2.1 Hz, 1H), 7.94 (d, J=2.1 Hz, 1H), 7.46-7.29 (m, 5H), 6.77 (dd, J=8.3, 2.2 Hz, 1H), 5.23-5.07 (m, 2H), 4.85 (d, J=10.8 Hz, 1H), 4.09-3.98 (m, 1H), 3.90 (d, J=1.7 Hz, 3H), 2.76-2.67 (m, 1H), 1.66 (s, 3H), 0.69-0.65 (m, 3H) ppm; NH₂ amide not observed. ESI-MS m/z calc. 579.17926, found 580.7 (M+1)⁺; 578.7 (M−1); Retention time: 3.7 minutes.

### Step 9:

A solution of 4-((2R,3S,4S,5R)-3-(5-(benzyloxy)-3,4-difluoro-2-methoxyphenyl)-4,5-dimethyl-5-(trifluoromethyl)tetrahydrofuran-2-carboxamido) picolinamide in ethyl acetate (5 mL) was added to Pd/C (100 mg, 0.047 mmol). The reaction mixture was degassed and stirred at ambient temperature under a hydrogen atmosphere. The mixture was filtered through a pad of celite, and washed with DCM. The filtrates were collected, concentrated in vacuo, and lyophilized from a 3:1 mixture of MeCN and H₂O to give 4-((2R,3S,4S,5R)-3-(3,4-difluoro-5-hydroxy-2-methoxyphenyl)-4,5-dimethyl-5-(trifluoromethyl)tetrahydrofuran-2-carboxamido) picolinamide (178, 22.8 mg, 12%). ¹H NMR (500 MHZ, DMSO-d₆) δ 10.74 (s, 1H), 10.02 (s, 1H), 8.50 (d, J=5.5 Hz, 1H), 8.29 (d, J=2.2 Hz, 1H), 8.06 (d, J=2.7 Hz, 1H), 7.84 (dd, J=5.5, 2.2 Hz, 1H), 7.61 (d, J=2.8 Hz, 1H), 6.68 (dd, J=8.9, 2.1 Hz, 1H), 4.94 (d, J=10.0 Hz, 1H), 4.25 (dd, J=10.0, 8.0 Hz, 1H), 3.80 (s, 3H), 2.82-2.71 (m, 1H), 1.60 (s, 3H), 0.78-0.73 (m, 3H) ppm. ESI-MS m/z calc. 489.13232, found 490.3 (M+1)⁺; 488.2 (M−1); Retention time: 2.85 minutes.

### Step 10:

3,3-Difluorocyclobutyl methanesulfonate (3 mg, 0.016 mmol) and K₂CO₃ (4.2 mg, 0.030 mmol) were successively added to a stirred solution of 4-((2R,3S,4S,5R)-3-(3,4-difluoro-5-hydroxy-2-methoxyphenyl)-4,5-dimethyl-5-(trifluoromethyl)tetrahydrofuran-2-carboxamido) picolinamide (5 mg, 0.010 mmol) in DMF (0.25 mL). The reaction mixture was heated to 110° C. in a sealed vial for 16 h. The mixture was quenched by addition of water (10 mL) and partitioned with DCM (10 mL). The aqueous layer was separated and extracted with DCM (10 mL). The combined organic extracts were dried over MgSO₄, filtered, and concentrated in vacuo. Purification by flash chromatography (4 g SiO₂, 0 to 100% AcOEt in heptane) gave 4-((2R,3S,4S,5R)-3-(5-(3,3-difluorocyclobutoxy)-3,4-difluoro-2-methoxyphenyl)-4,5-dimethyl-5-(trifluoromethyl)tetrahydrofuran-2-carboxamido) picolinamide (179, 0.8 mg, 13%). ESI-MS m/z calc. 579.1604, found 580.6 (M+1)⁺; 578.6 (M−1); Retention time: 3.64 minutes.

### Example 19

### 4-((2R,3S,4S,5R)-3-(4-(difluoromethoxy)-3-fluoro-2-methoxyphenyl)-4,5-dimethyl-5-(trifluoromethyl)tetrahydrofuran-2-carboxamido) picolinamide (180)

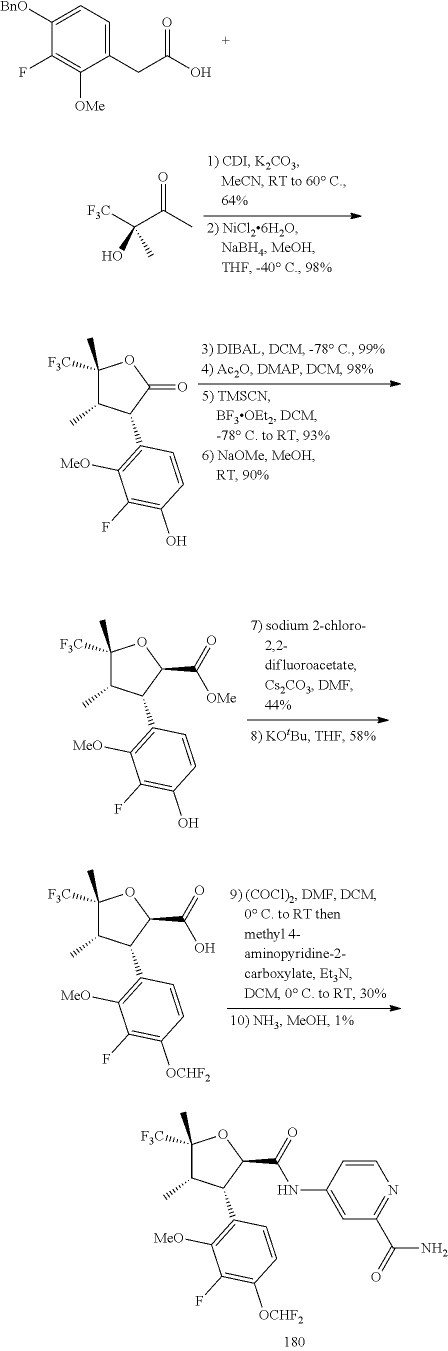

### Step 1:

CDI (6 g, 37.003 mmol) was added to a solution of 2-(4-(benzyloxy)-3-fluoro-2-methoxyphenyl) acetic acid (Intermediate B, 9.8 g, 32.072 mmol) in acetonitrile (100 mL) and the mixture was stirred for 15 min at 40° C. (R)-4,4,4-trifluoro-3-hydroxy-3-methylbutan-2-one (Intermediate C, 6 g, 38.436 mmol) and potassium carbonate (5.5 g, 39.796 mmol) were added and the stirring was continued at 60° C. for 30 h. The reaction mixture was diluted with water (50 mL) and extracted with MTBE (2×100 mL). The organic layer was washed with 2 M hydrochloric acid (2×50 mL) then dried over Na₂SO₄, filtered, and concentrated in vacuo. Purification by flash chromatography (120 g SiO₂, 0 to 100% ethyl acetate in heptane) gave (R)-3-(4-(benzyloxy)-3-fluoro-2-methoxyphenyl)-4,5-dimethyl-5-(trifluoromethyl) furan-2 (5H)-one (9.17 g, 64%) as a yellow solid. ¹H NMR (400 MHZ, Chloroform-d) δ 7.46-7.34 (m, 5H), 6.91 (dd, J=8.7, 1.8 Hz, 1H), 6.80 (dd, J=8.7, 7.8 Hz, 1H), 5.16 (s, 2H), 3.85 (d, J=1.8 Hz, 3H), 2.03 (s, 3H), 1.73 (s, 3H) ppm. ESI-MS m/z calc. 410.1141, found 411.23 (M+1)⁺; Retention time: 2.97 minutes.

### Step 2:

Nickel dichloride hexahydrate (1.8 g, 7.573 mmol) was added to a stirred and previously degassed solution of (R)-3-(4-(benzyloxy)-3-fluoro-2-methoxyphenyl)-4,5-dimethyl-5-(trifluoromethyl) furan-2 (5H)-one (3 g, 7.311 mmol) in MeOH (300 mL) and THF (60 mL) at −40° C. NaBH₄ (1.4 g, 37.00 mmol) was added portionwise and the reaction mixture was stirred until completion. A total of 7 eq of NiCl₂·6H₂O were added. A saturated ammonium chloride solution (100 mL) was added and the mixture partitioned with DCM (100 mL). The organic phase was separated, dried over MgSO₄, filtered, and concentrated in vacuo to give a mixture of stereoisomers with (3S,4S,5R)-3-(3-fluoro-4-hydroxy-2-methoxyphenyl)-4,5-dimethyl-5-(trifluoromethyl)dihydrofuran-2 (3H)-one (2.3 g, 98%) as the main diastereoisomer, which was used in the next step without further purification. ESI-MS m/z calc. 322.08282, found 321.4 (M−1); Retention time: 0.79 minutes.

### Step 3:

DIBAL (15 mL of 1 M in DCM, 15.00 mmol) was added dropwise to a stirred solution of (3S,4S,5R)-3-(3-fluoro-4-hydroxy-2-methoxyphenyl)-4,5-dimethyl-5-(trifluoromethyl)dihydrofuran-2 (3H)-one (2.3 g, 7.137 mmol) in DCM (40 mL) at −78° C. under a nitrogen atmosphere. The reaction mixture was stirred at −78° C. Upon reaction completion, the mixture was quenched by addition of a saturated ammonium chloride solution and a Rochelle's salt (30% w/w) solution (100 mL, 1:1). The resulting mixture was vigorously stirred at ambient temperature until a clear phase separation was achieved. The organic extracts were dried over MgSO₄, filtered, and concentrated in vacuo to give a mixture of stereoisomers with (3S,4S,5R)-3-(3-fluoro-4-hydroxy-2-methoxyphenyl)-4,5-dimethyl-5-(trifluoromethyl)tetrahydrofuran-2-ol (2.3 g, 99%) as the main diastereoisomer, which was used in the next step without further purification. ESI-MS m/z calc. 324.09848, found 323.4 (M−1); Retention time: 0.73 minutes.

### Step 4:

Acetic anhydride (700 μL, 7.419 mmol) was added to a stirred solution of (3S,4S,5R)-3-(3-fluoro-4-hydroxy-2-methoxyphenyl)-4,5-dimethyl-5-(trifluoromethyl)tetrahydrofuran-2-ol (380 mg, 1.172 mmol) and DMAP (210 mg, 1.719 mmol) in DCM (4 mL) at room temperature and under nitrogen atmosphere. The reaction mixture was stirred at ambient temperature. Upon reaction completion, the mixture was quenched by addition of saturated sodium bicarbonate solution (30 mL). The mixture was diluted with DCM (20 mL). The aqueous phase was separated and extracted with DCM (10 mL). The combined organic extracts were dried over MgSO₄, filtered, and concentrated in vacuo. Purification by flash chromatography (24 g SiO₂, 0 to 100% AcOEt in heptane) gave a mixture of stereoisomers with (3S,4S,5R)-3-(4-acetoxy-3-fluoro-2-methoxyphenyl)-4,5-dimethyl-5-(trifluoromethyl)tetrahydrofuran-2-yl acetate as the main diastereomer (470 mg, 98%) and a mixture of epimers at the C₂ position, which was used in the next step without further purification. ESI-MS m/z calc. 408.1196, found 407.3 (M−1); Retention time: 1.01 minutes.

### Step 5:

TMSCN (400 μL, 3.000 mmol) and BF₃·OEt₂ (1000 μL, 8.103 mmol) were successively added to a stirred solution of (3S,4S,5R)-3-(4-acetoxy-3-fluoro-2-methoxyphenyl)-4,5-dimethyl-5-(trifluoromethyl)tetrahydrofuran-2-yl acetate (470 mg, 1.151 mmol) in DCM (15 mL) at −78° C. The reaction mixture was stirred at −78° C. for 30 min before being warmed to ambient temperature. Upon completion of the reaction, the mixture was quenched with a saturated sodium bicarbonate solution (60 mL). The mixture was extracted with DCM (3×30 mL). The combined organic extracts were dried over Na₂SO₄, filtered, and concentrated in vacuo to give a mixture of stereoisomers with 4-((3S,4S,5R)-2-cyano-4,5-dimethyl-5-(trifluoromethyl)tetrahydrofuran-3-yl)-2-fluoro-3-methoxyphenyl acetate (400 mg, 93%) as the main diastereoisomer. ESI-MS m/z calc. 375.10938, found 374.5 (M−1); Retention time: 1.0 minutes.

### Step 6:

4-((3S,4S,5R)-2-Cyano-4,5-dimethyl-5-(trifluoromethyl)tetrahydrofuran-3-yl)-2-fluoro-3-methoxyphenyl acetate was directly dissolved in MeOH (7 mL). A 0.5 M sodium methoxide solution (800 μL of 25% w/w in MeOH, 3.498 mmol) was added and the reaction mixture was stirred under nitrogen at ambient temperature overnight. Upon complete conversion to the corresponding imidate 2-fluoro-4-((3S,4S,5R)-2-(imino(methoxy)methyl)-4,5-dimethyl-5-(trifluoromethyl)tetrahydrofuran-3-yl)-3-methoxyphenyl acetate; (ESI-MS m/z calc. 365.12503, Retention time: 0.81 minutes), the mixture was quenched by addition of a saturated citric acid solution. The reaction mixture was stirred at ambient temperature for an additional 4 h. The mixture was extracted with DCM (2×30 mL). The combined organic extracts were dried over MgSO₄, filtered and concentrated in vacuo to give a mixture of stereoisomers with methyl(2R,3S,4S,5R)-3-(3-fluoro-4-hydroxy-2-methoxyphenyl)-4,5-dimethyl-5-(trifluoromethyl)tetrahydrofuran-2-carboxylate (380 mg, 90%) as the main diastereoisomer and containing minor impurities. ESI-MS m/z calc. 366.10904, found 365.4 (M−1); Retention time: 0.87 minutes.

### Step 7:

Sodium 2-chloro-2,2-difluoroacetate (1.1 g, 7.168 mmol) was added to a mixture of methyl(2R,3S,4S,5R)-3-(3-fluoro-4-hydroxy-2-methoxyphenyl)-4,5-dimethyl-5-(trifluoromethyl)tetrahydrofuran-2-carboxylate (1.01 g, 2.757 mmol) and Cs₂CO₃ (2.7 g, 8.287 mmol) in DMF (10 mL). The reaction mixture was heated to 90° C. Upon completion of the reaction, the mixture was diluted with DCM (20 mL) and partitioned with water (50 mL). The organic phase was separated, dried over MgSO₄, filtered, and concentrated in vacuo. Purification by flash chromatography (12 g SiO₂, 0 to 100% AcOEt in heptane) gave methyl(2R,3S,4S,5R)-3-(4-(difluoromethoxy)-3-fluoro-2-methoxyphenyl)-4,5-dimethyl-5-(trifluoromethyl)tetrahydrofuran-2-carboxylate (500 mg, 44%). ESI-MS m/z calc. 416.10583, Retention time: 0.87 minutes; no mass ionisation.

### Step 8:

Potassium tert-butoxide (200 mg, 1.782 mmol) was added to a stirred solution of methyl(2R,3S,4S,5R)-3-(4-(difluoromethoxy)-3-fluoro-2-methoxyphenyl)-4,5-dimethyl-5-(trifluoromethyl)tetrahydrofuran-2-carboxylate (180 mg, 0.4324 mmol) in THF (3 mL) at ambient temperature. The reaction mixture was stirred at ambient temperature for 5 min. The mixture was quenched by addition of a saturated ammonium chloride solution (3 mL) and diluted with DCM (3 mL). The organic phase was separated, washed with DCM (5 mL), dried over MgSO₄, filtered, and concentrated in vacuo to give (2R,3S,4S,5R)-3-(4-(difluoromethoxy)-3-fluoro-2-methoxyphenyl)-4,5-dimethyl-5-(trifluoromethyl)tetrahydrofuran-2-carboxylic acid (100 mg, 58%), which was used in the next step without further purification. ESI-MS m/z calc. 402.09018, found 401.4 (M−1); Retention time: 0.6 minutes.

### Step 9:

Oxalyl chloride (25 μL, 0.287 mmol) was added dropwise to a stirred solution of (2R,3S,4S,5R)-3-(4-(difluoromethoxy)-3-fluoro-2-methoxyphenyl)-4,5-dimethyl-5-(trifluoromethyl)tetrahydrofuran-2-carboxylic acid (50 mg, 0.124 mmol) and DMF (2 μL, 0.026 mmol) in DCM (500 μL) at ambient temperature. The reaction mixture was stirred at ambient temperature for 30 min. The solution was concentrated in vacuo. The residue, dissolved in a mixture of DCM (500 μL) and NMP (added dropwise until the starting material fully dissolved), was added to a stirred solution of methyl 4-aminopyridine-2-carboxylate (25 mg, 0.164 mmol) and triethylamine (25 μL, 0.179 mmol) in DCM (500 μL) at ambient temperature. The mixture was stirred for 1 h before quenching with methanol (100 μL). The mixture was subsequently concentrated in vacuo. Purification by flash chromatography gave methyl 4-((2R,3S,4S,5R)-3-(4-(difluoromethoxy)-3-fluoro-2-methoxyphenyl)-4,5-dimethyl-5-(trifluoromethyl)tetrahydrofuran-2-carboxamido) picolinate (20 mg, 30%), which was immediately taken as is in the next step. ESI-MS m/z calc. 536.1382, found 537.6 (M+1)⁺; 535.5 (M−1); Retention time: 0.97 minutes.

### Step 10:

Methanolic ammonia (4 mL of 7 M, 28.00 mmol) was added to methyl 4-((2R,3S,4S,5R)-3-(4-(difluoromethoxy)-3-fluoro-2-methoxyphenyl)-4,5-dimethyl-5-(trifluoromethyl)tetrahydrofuran-2-carboxamido) picolinate (20 mg). The reaction mixture was stirred at ambient temperature for 4 h. The mixture was concentrated in vacuo. Purification by flash chromatography (4 g SiO₂, 0 to 100% AcOEt in heptane) followed by a purification by chiral SFC using a Chiralcel OD-H column, 5 μm particle size, 25 cm×10 mm from Daicel (Mobile phase: 15% methanol (supplemented with 20 mM NH₃), 85% CO₂; System pressure: 100 bar) on a Minigram SFC instrument from Berger Instruments gave 4-((2R,3S,4S,5R)-3-(4-(difluoromethoxy)-3-fluoro-2-methoxyphenyl)-4,5-dimethyl-5-(trifluoromethyl)tetrahydrofuran-2-carboxamido) picolinamide (180, 1 mg, 1%) as a white solid. ¹H NMR (500 MHz, DMSO-d₆) δ 10.73 (s, 1H), 8.50 (d, J=5.4 Hz, 1H), 8.29 (d, J=2.1 Hz, 1H), 8.06 (d, J=2.9 Hz, 1H), 7.84 (dd, J=5.5, 2.2 Hz, 1H), 7.63-7.59 (m, 1H), 7.25 (t, J=73.2 Hz, 1H), 7.21-7.15 (m, 1H), 7.10 (t, J=8.0 Hz, 1H), 5.12 (d, J=10.2 Hz, 1H), 4.28 (dd, J=10.2, 7.7 Hz, 1H), 3.93 (d, J=1.9 Hz, 3H), 2.79 (p, J=7.5 Hz, 1H), 1.62 (s, 3H), 0.77-0.72 (m, 3H) ppm. ESI-MS m/z calc. 521.13855, found 522.1 (M+1)⁺; 520.1 (M−1); Retention time: 3.29 minutes.

### Example 20

### 4-((2R,3S,4S,5R)-3-(2-(2-(2-oxa-6-azaspiro[3.3]heptan-6-yl)ethoxy)-3,4-difluorophenyl)-4,5-dimethyl-5-(trifluoromethyl)tetrahydrofuran-2-carboxamido)-5-methylpicolinamide (181)

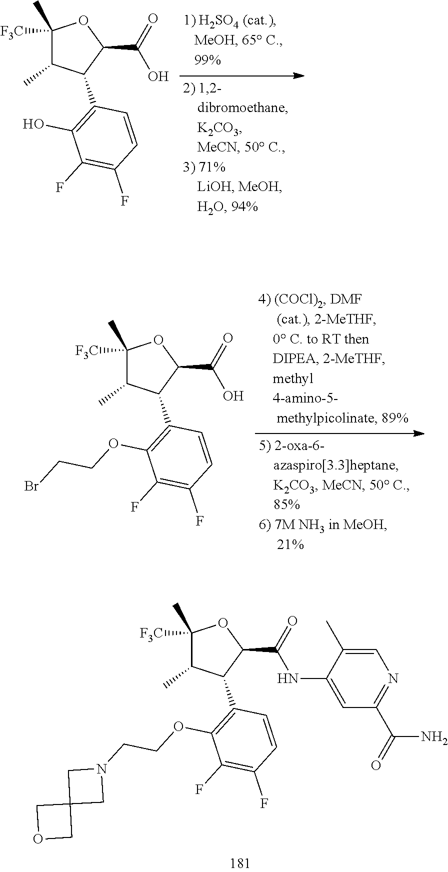 [SMILES: C[C@H]1[C@@H](c2ccc(F)c(F)c2O)[C@H](C(=O)O)O[C@@]1(C)C(F)(F)F ; C[C@H]1[C@@H](c2ccc(F)c(F)c2OCCBr)[C@H](C(=O)O)O[C@@]1(C)C(F)(F)F ; Cc1cnc(C(N)=O)cc1NC(=O)[C@@H]1O[C@@](C)(C(F)(F)F)[C@@H](C)[C@H]1c1ccc(F)c(F)c1OCCN1CC2(COC2)C1]

### Step 1:

H₂SO₄ (20 μL, 0.3752 mmol) was added to a stirred solution of (2R,3S,4S,5R)-3-(3,4-difluoro-2-hydroxyphenyl)-4,5-dimethyl-5-(trifluoromethyl)tetrahydrofuran-2-carboxylic acid (Product of Example 6, Step 1, 700 mg, 2.057 mmol) in MeOH (6 mL). The reaction mixture was stirred at reflux for 2 h. The mixture was cooled down to ambient temperature and concentrated in vacuo. The residue was partitioned between EtOAc and water. The organic phase was separated, dried over Na₂SO₄, filtered, and concentrated in vacuo to give methyl(2R,3S,4S,5R)-3-(3,4-difluoro-2-hydroxyphenyl)-4,5-dimethyl-5-(trifluoromethyl)tetrahydrofuran-2-carboxylate (756 mg, 99%) as a colourless oil, which was taken to the next step without further purification. ¹H NMR (500 MHZ, Chloroform-d) δ 6.95 (ddd, J=8.1, 5.3, 2.2 Hz, 1H), 6.75 (td, J=9.3, 7.7 Hz, 1H), 5.60 (s, 1H), 4.99 (d, J=10.0 Hz, 1H), 4.25-4.16 (m, 1H), 3.74 (s, 3H), 2.86 (p, J=7.6 Hz, 1H), 1.65 (s, 3H), 0.82-0.80 (m, 3H) ppm. ESI-MS m/z calc. 354.08905, found 353.0 (M−1); Retention time: 2.05 minutes.

### Step 2:

K₂CO₃ (900 mg, 6.512 mmol) and 1,2-dibromoethane (1.2 mL, 13.93 mmol) were successively added to a stirred solution of methyl(2R,3S,4S,5R)-3-(3,4-difluoro-2-hydroxyphenyl)-4,5-dimethyl-5-(trifluoromethyl)tetrahydrofuran-2-carboxylate (756 mg, 2.134 mmol) in MeCN (20 mL) at ambient temperature under a nitrogen atmosphere. The reaction mixture was heated at 50° C. After 3 h 30 min, the mixture was cooled to ambient temperature. The mixture was partitioned between EtOAc and water. The aqueous phase was separated and extracted with EtOAc. The combined organic extracts were washed with brine, dried over Na₂SO₄, filtered, and concentrated in vacuo. Purification by flash chromatography (SiO₂, 0 to 40% EtOAc in heptane) gave methyl(2R,3S,4S,5R)-3-(2-(2-bromoethoxy)-3,4-difluorophenyl)-4,5-dimethyl-5-(trifluoromethyl)tetrahydrofuran-2-carboxylate (703 mg, 71%) as a colourless oil. ¹H NMR (500 MHZ, DMSO-d₆) δ 7.25 (ddd, J=7.9, 5.8, 1.9 Hz, 1H), 7.17 (td, J=9.5, 7.6 Hz, 1H), 5.16 (d, J=10.6 Hz, 1H), 4.62-4.58 (m, 1H), 4.40-4.30 (m, 2H), 4.00-3.77 (m, 2H), 3.62 (s, 3H), 2.76 (p, J=7.5 Hz, 1H), 1.57 (s, 3H), 0.79-0.57 (m, 3H) ppm. ESI-MS m/z calc. 460.03085, Retention time: 1.07 minutes; no mass ionisation.

### Step 3:

LiOH monohydrate (195 mg, 4.647 mmol) added to a stirred solution of methyl(2R,3S,4S,5R)-3-(2-(2-bromoethoxy)-3,4-difluorophenyl)-4,5-dimethyl-5-(trifluoromethyl)tetrahydrofuran-2-carboxylate (703 mg, 1.524 mmol) in a mixture of MeOH (15 mL) and water (3 mL) at ambient temperature. After stirring for 1 h, the reaction mixture was concentrated in vacuo and the residue partitioned between EtOAc and 1M HCl. The aqueous phase was separated and extracted twice with EtOAc. The combined organic extracts were dried over Na₂SO₄, filtered, and concentrated in vacuo to give (2R,3S,4S,5R)-3-(2-(2-bromoethoxy)-3,4-difluorophenyl)-4,5-dimethyl-5-(trifluoromethyl)tetrahydrofuran-2-carboxylic acid (710 mg, 94%) as a colourless oil. ¹H NMR (500 MHZ, DMSO-d₆) δ 12.88 (s, 1H), 7.33-6.91 (m, 2H), 4.99 (d, J=10.7 Hz, 1H), 4.58 (dddd, J=11.2, 5.5, 3.9, 1.5 Hz, 1H), 4.45-4.34 (m, 1H), 4.28 (dd, J=10.7, 7.4 Hz, 1H), 3.90-3.78 (m, 2H), 2.76 (p, J=7.5 Hz, 1H), 1.56 (s, 3H), 0.68 (dt, J=7.3, 2.4 Hz, 3H) ppm. ESI-MS m/z calc. 446.0152, found 445.0 (M−1); Retention time: 0.62 minutes.

### Step 4:

A solution of (2R,3S,4S,5R)-3-(2-(2-bromoethoxy)-3,4-difluorophenyl)-4,5-dimethyl-5-(trifluoromethyl)tetrahydrofuran-2-carboxylic acid (144 mg, 0.290 mmol) in 2-MeTHF (1.5 mL) was placed under a nitrogen atmosphere and the solution was cooled with an ice bath. DMF (5 μL, 0.065 mmol) and oxalyl chloride (60 μL, 0.688 mmol) were successively added and the cooling bath was removed. The reaction mixture was stirred for 1 h at ambient temperature, and then concentrated in vacuo. The residue, dissolved in 2-MeTHF (1.5 mL), was added to a stirred solution of methyl 4-amino-5-methylpicolinate (Intermediate N, 85 mg, 0.512 mmol) and DIPEA (150 μL, 0.861 mmol) in 2-MeTHF (1.5 mL) at ambient temperature. The reaction mixture was stirred for 30 min, then partitioned between EtOAc and water. The aqueous phase was separated and extracted with EtOAc. The combined organic extracts were dried over Na₂SO₄, filtered and concentrated in vacuo. Purification by flash chromatography (SiO₂, 0 to 100% EtOAc in heptane) gave methyl 4-((2R,3S,4S,5R)-3-(2-(2-bromoethoxy)-3,4-difluorophenyl)-4,5-dimethyl-5-(trifluoromethyl)tetrahydrofuran-2-carboxamido)-5-methylpicolinate (153 mg, 89%) as a colourless oil, which was used in the next step without further characterisation. ESI-MS m/z calc. 594.07886, found 595.0 (M+1)⁺; 593.0 (M−1); Retention time: 1.03 minutes.

### Step 5:

K₂CO₃ (150 mg, 1.085 mmol) and 2-oxa-6-azaspiro[3.3]heptane (150 mg, 1.513 mmol) were successively added to a stirred solution of methyl 4-((2R,3S,4S,5R)-3-(2-(2-bromoethoxy)-3,4-difluorophenyl)-4,5-dimethyl-5-(trifluoromethyl)tetrahydrofuran-2-carboxamido)-5-methylpicolinate (150 mg, 0.252 mmol) in MeCN (2 mL) under a nitrogen atmosphere. The reaction mixture was stirred at 50° C. for 6 h. The mixture was cooled to ambient temperature and partitioned between EtOAc and water. The aqueous phase was separated and extracted with EtOAc. The combined organic extracts were dried over Na₂SO₄, filtered, and concentrated in vacuo to give methyl 4-((2R,3S,4S,5R)-3-(2-(2-(2-oxa-6-azaspiro[3.3]heptan-6-yl)ethoxy)-3,4-difluorophenyl)-4,5-dimethyl-5-(trifluoromethyl)tetrahydrofuran-2-carboxamido)-5-methylpicolinate (138 mg, 85%) as a colourless film, which was used in the next step without further purification or characterisation. ESI-MS m/z calc. 613.2211, found 614.0 (M+1)⁺; 612.0 (M−1); Retention time: 0.9 minutes.

### Step 6:

Methyl 4-((2R,3S,4S,5R)-3-(2-(2-(2-oxa-6-azaspiro[3.3]heptan-6-yl)ethoxy)-3,4-difluorophenyl)-4,5-dimethyl-5-(trifluoromethyl)tetrahydrofuran-2-carboxamido)-5-methylpicolinate (138 mg, 0.214 mmol) was stirred in a sealed tube in a methanolic ammonia solution (3 mL of 4 M, 12.00 mmol) at 50° C. overnight. The reaction mixture was cooled to ambient temperature. Purification by reversed phase HPLC using a X-bridge C18 column (150×19 mm, 5 mm particle size) from Waters (Mobile phase: acetonitrile in water, supplemented with 0.1% ammonium hydroxide) gave a white solid, which was further purified by flash chromatography (SiO₂, 80 to 100% 3:1 EtOAc: EtOH in heptane) to give 4-((2R,3S,4S,5R)-3-(2-(2-(2-oxa-6-azaspiro[3.3]heptan-6-yl)ethoxy)-3,4-difluorophenyl)-4,5-dimethyl-5-(trifluoromethyl)tetrahydrofuran-2-carboxamido)-5-methylpicolinamide (181, 26.62 mg, 21%) as a white solid. ¹H NMR (500 MHZ, DMSO-d₆) δ 9.68 (s, 1H), 8.43 (s, 1H), 8.39 (s, 1H), 8.01 (d, J=2.8 Hz, 1H), 7.55 (d, J=2.8 Hz, 1H), 7.27 (ddd, J=8.1, 5.9, 1.7 Hz, 1H), 7.22-7.14 (m, 1H), 5.32 (d, J=10.9 Hz, 1H), 4.54 (s, 4H), 4.36 (dd, J=10.9, 7.1 Hz, 1H), 4.15-4.06 (m, 1H), 3.96 (dt, J=10.4, 5.0 Hz, 1H), 2.88 (p, J=7.4 Hz, 1H), 2.64 (t, J=5.1 Hz, 2H), 2.25 (s, 3H), 1.66 (s, 3H), 0.87-0.61 (m, 3H) ppm; 4H not observed, possibly obscured by water peak. ESI-MS m/z calc. 598.22144, found 599.0 (M+1)⁺; 597.0 (M−1); Retention time: 3.05 minutes.

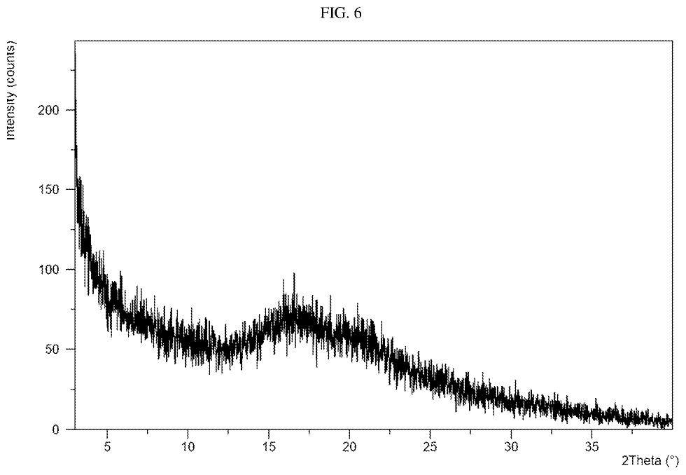

Compound 181 was analyzed by X-ray powder diffraction and determined to be amorphous (see FIG. 6).

### Example 21

### 4-((2R,3S,4S,5R)-3-(2-(2-cyclopropyl-2-(hydroxyimino)ethoxy)-3,4-difluorophenyl)-4,5-dimethyl-5-(trifluoromethyl)tetrahydrofuran-2-carboxamido) picolinamide (182)

### rel-4-((2R*,3S*,4S*,5R*)-3-(2-(2-cyclopropyl-2-(hydroxyimino)ethoxy)-3,4-difluorophenyl)-4,5-dimethyl-5-(trifluoromethyl)tetrahydrofuran-2-carboxamido) picolinamide (183) and

### rel-4-((2R*,3S*,4S*,5R*)-3-(2-(2-cyclopropyl-2-(hydroxyimino)ethoxy)-3,4-difluorophenyl)-4,5-dimethyl-5-(trifluoromethyl)tetrahydrofuran-2-carboxamido) picolinamide (184)

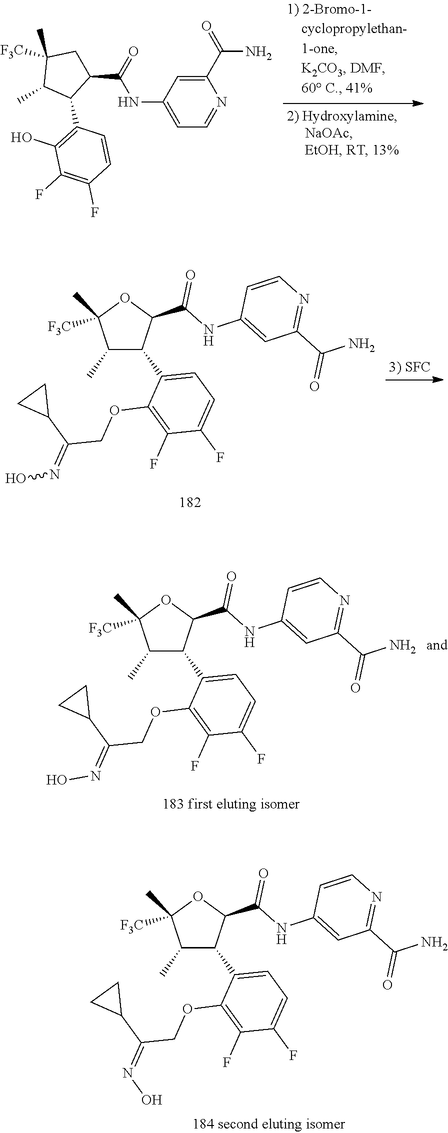 [SMILES: C[C@H]1[C@@H](c2ccc(F)c(F)c2O)[C@H](C(=O)Nc2ccnc(C(N)=O)c2)C[C@@]1(C)C(F)(F)F ; C[C@H]1[C@@H](c2ccc(F)c(F)c2OCC(=NO)C2CC2)[C@H](C(=O)Nc2ccnc(C(N)=O)c2)O[C@@]1(C)C(F)(F)F ; C[C@H]1[C@@H](c2ccc(F)c(F)c2OC/C(=N/O)C2CC2)[C@H](C(=O)Nc2ccnc(C(N)=O)c2)O[C@@]1(C)C(F)(F)F ; "and" ; C[C@H]1[C@@H](c2ccc(F)c(F)c2OC/C(=N\O)C2CC2)[C@H](C(=O)Nc2ccnc(C(N)=O)c2)O[C@@]1(C)C(F)(F)F]

### Step 1:

K₂CO₃ (31 mg, 0.224 mmol) was added to a stirred solution of 4-((2R,3S,4S,5R)-3-(3,4-difluoro-2-hydroxyphenyl)-4,5-dimethyl-5-(trifluoromethyl)tetrahydrofuran-2-carboxamido) picolinamide (Product of Example 2, Step 10, 54 mg, 0.118 mmol) in DMF (1.5 mL). The vial was sealed and placed under a nitrogen atmosphere. A solution of 2-bromo-1-cyclopropylethan-1-one (22 mg, 0.135 mmol) in DMF (0.5 mL) was added in one portion and the reaction mixture was stirred at 60° C. for 2 h. The reaction mixture was cooled to ambient temperature overnight. The reaction mixture was diluted with a saturated aqueous sodium bicarbonate solution (15 mL) and extracted with EtOAc (3×15 mL). The combined organic extracts were washed with brine (20 mL), dried over MgSO₄, filtered, and concentrated in vacuo to give 4-((2R,3S,4S,5R)-3-(2-(2-cyclopropyl-2-oxoethoxy)-3,4-difluorophenyl)-4,5-dimethyl-5-(trifluoromethyl)tetrahydrofuran-2-carboxamido) picolinamide (51 mg, 41%) as an orange gum. ESI-MS m/z calc. 541.16364, found 542.3 (M+1)⁺; 540.3 (M−1); Retention time: 0.93 minutes.

### Step 2 and 3:

Hydroxylamine hydrochloride (21 mg, 0.302 mmol) and sodium acetate (20 mg, 0.244 mmol) were successively added to a solution of 4-((2R,3S,4S,5R)-3-(2-(2-cyclopropyl-2-oxoethoxy)-3,4-difluorophenyl)-4,5-dimethyl-5-(trifluoromethyl)tetrahydrofuran-2-carboxamido) picolinamide (50 mg, 0.092 mmol) in EtOH (2 mL). The reaction mixture was stirred at ambient temperature under nitrogen for 18 h. The reaction mixture was then partitioned between water (15 mL) and DCM (20 mL) and stirred vigorously for 45 min. The mixture was passed through a phase separation cartridge and concentrated in vacuo to give an orange, glassy solid. Purification by flash chromatography (4 g SiO₂, 10 to 90% EtOAc in heptane) followed by reverse phase HPLC using a X-bridge C18 column (150×19 mm, 5 mm particle size) from Waters (Gradient: 47.4% to 94.7% acetonitrile in water (supplemented with 0.1% ammonium hydroxide); over 9 min; Flow rate: 19 mL/min; sample dissolved in acetonitrile and injected at 1 mL/min) gave 4-((2R,3S,4S,5R)-3-(2-(2-cyclopropyl-2-(hydroxyimino)ethoxy)-3,4-difluorophenyl)-4,5-dimethyl-5-(trifluoromethyl)tetrahydrofuran-2-carboxamido) picolinamide (182, 6.8 mg, 13%) as a white solid, which was a mixture of geometric isomers having E and Z configuration at the 2-cyclopropyl-2-(hydroxyimino)ethoxy group. ¹H NMR (500 MHZ, Methanol-d₄) δ 8.53-8.43 (m, 1H), 8.25 (t, J=2.0 Hz, 1H), 7.89 (ddd, J=5.5, 2.2, 0.9 Hz, 1H), 7.22-7.10 (m, 1H), 7.10-6.94 (m, 1H), 5.07 (dd, J=10.5, 8.6 Hz, 1H), 4.91 (dd, J=12.5, 1.2 Hz, 0.5H), 4.49-4.42 (m, 0.5H), 4.41 (s, 1.5H), 4.33 (dd, J=10.6, 7.8 Hz, 0.5H), 2.91-2.78 (m, 1H), 2.30 (tt, J=8.6, 5.4 Hz, 0.5H), 1.76 (tt, J=7.9, 5.9 Hz, 0.5H), 1.66 (m, 3H), 1.05-0.86 (m, 2H), 0.86-0.70 (m, 5H) ppm; amides NH and NH₂ not observed. ESI-MS m/z calc. 556.1745, found 557.6 (M+1)⁺; Retention time: 3.25 and 3.28 minutes.

The geometric E and Z isomers of 4-((2R,3S,4S,5R)-3-(2-(2-cyclopropyl-2-(hydroxyimino)ethoxy)-3,4-difluorophenyl)-4,5-dimethyl-5-(trifluoromethyl)tetrahydrofuran-2-carboxamido) picolinamide (5 mg) were separated by chiral SFC using a Chiralcel OJ-H column, 5 μm particle size, 25 cm×10 mm from Daicel Corporation (Mobile phase: 15% methanol (supplemented with 20 mM NH₃), 85% CO₂; System pressure: 100 bar) on a Minigram SFC instrument from Berger Instruments to give:

First Eluting Isomer (rt=4.39 min): rel-4-((2R*,3S*,4S*,5R*)-3-(2-(2-cyclopropyl-2-(hydroxyimino)ethoxy)-3,4-difluorophenyl)-4,5-dimethyl-5-(trifluoromethyl)tetrahydrofuran-2-carboxamido) picolinamide (183, 1.5 mg, 3%). ¹H NMR (500 MHZ, Methanol-d₄) δ 8.48 (dd, J=5.6, 0.6 Hz, 1H), 8.28-8.20 (m, 1H), 7.90 (dd, J=5.5, 2.2 Hz, 1H), 7.23-7.11 (m, 1H), 7.03 (td, J=9.4, 7.6 Hz, 1H), 5.08 (d, J=10.5 Hz, 1H), 4.91 (dd, J=12.4, 1.2 Hz, 1H), 4.55 (s, 1H), 4.44 (dd, J=10.4, 7.9 Hz, 1H), 2.83 (p, J=7.6 Hz, 1H), 1.76 (tt, J=7.7, 5.4 Hz, 1H), 1.65 (s, 3H), 0.87-0.77 (m, 4H), 0.77-0.69 (m, 3H) ppm; amides NH and NH₂ not observed. ESI-MS m/z calc. 556.1745, found 557.2 (M+1)⁺; Retention time: 3.28 minutes.

Second Eluting Isomer (rt=5.11 min): rel-4-((2R*,3S*,4S*,5R*)-3-(2-(2-cyclopropyl-2-(hydroxyimino)ethoxy)-3,4-difluorophenyl)-4,5-dimethyl-5-(trifluoromethyl)tetrahydrofuran-2-carboxamido) picolinamide (184, 1.5 mg, 3%). ¹H NMR (500 MHZ, Methanol-d₄) δ 8.48 (d, J=5.5 Hz, 1H), 8.25 (d, J=2.1 Hz, 1H), 7.90 (dd, J=5.5, 2.2 Hz, 1H), 7.15 (ddd, J=8.3, 5.5, 2.1 Hz, 1H), 7.01 (td, J=9.6, 7.8 Hz, 1H), 5.06 (d, J=10.6 Hz, 1H), 4.41 (s, 2H), 4.33 (dd, J=10.6, 7.7 Hz, 1H), 2.83 (p, J=7.6 Hz, 1H), 2.30 (tt, J=8.6, 5.4 Hz, 1H), 1.67 (s, 3H), 1.05-0.85 (m, 4H), 0.80 (dt, J=7.5, 2.5 Hz, 3H) ppm; amides NH and NH₂ not observed. ESI-MS m/z calc. 556.1745, found 557.7 (M+1)⁺; Retention time: 3.25 minutes.

The following compounds were made using the method described in Example 21, except that different alkylating agents were used in step 1. In the case of compounds 185, 186, 189, 190, and 191, step 3 was not carried out. In the case of compound 187 and 188, the purification step 3 was performed by chiral SFC using a Lux i-Cellulose-5 column, 5 μm particle size, 25 cm×10 mm from Phenomenex, Inc. (Mobile phase: 15% methanol (supplemented with 20 mM NH₃), 85% CO₂; System pressure: 100 bar) on a Minigram SFC instrument from Berger Instruments:

| Cmpd No. | Compound Name | LC/MS | NMR (shifts in ppm) |
|---|---|---|---|
| 185 | 4-((2R,3S,4S,5R)-3-(3,4- | ESI-MS m/z calc. | ¹H NMR (500 MHz, |
| | difluoro-2-((E)-2- | 530.1589, found 531.6 | Methanol-d₄) δ 8.48 (dd, |
| | (hydroxyimino)propoxy)phenyl)- | (M + 1)⁺; Retention time: | J = 5.6, 0.6 Hz, 1H), 8.25 |
| | 4,5-dimethyl-5- | 3.08 minutes | (dd, J = 2.2, 0.6 Hz, 1H), |
| | (trifluoromethyl)tetrahydrofuran- | | 7.90 (dd, J = 5.5, 2.2 Hz, |
| | 2-carboxamido)picolinamide | | 1H), 7.15 (ddd, J = 8.1, |
| | | | 5.5, 2.1 Hz, 1H), 7.02 |
| | | | (ddd, J = 9.9, 8.9, 7.5 Hz, |
| | | | 1H), 5.07 (d, J = 10.5 Hz, |
| | | | 1H), 4.65 (d, J = 1.1 Hz, |
| | | | 2H), 4.34 (dd, J = 10.5, |
| | | | 7.8 Hz, 1H), 2.79 (p, J = |
| | | | 7.6 Hz, 1H), 2.02 (s, 3H), |
| | | | 1.65 (d, J = 1.2 Hz, 3H), |
| | | | 0.81 (dt, J = 7.6, 2.3 Hz, |
| | | | 3H) ppm; NH and NH₂ |
| | | | amides and OH alcohol |
| | | | not observed. |
| 186 | 4-((2R,3S,4S,5R)-3-(3,4- | ESI-MS m/z calc. | ¹H NMR (500 MHz, |
| | difluoro-2-(2-(hydroxyimino)- | 558.1902, found 559.7 | Methanol-d₄) δ 8.50 |
| | 3-methylbutoxy)phenyl)-4,5- | (M + 1)⁺; Retention time: | (d, J = 5.5 Hz, 1H), 8.29- |
| | dimethyl-5- | 3.42 and 3.45 minutes | 8.24 (m, 1H), 7.95- |
| | (trifluoromethyl)tetrahydrofuran- | | 7.89 (m, 1H), 7.22-7.14 |
| | 2-carboxamido)picolinamide | | (m, 1H), 7.09-7.00 (m, |
| | (Mixture of geometric isomers | | 1H), 5.13-5.07 (m, 1H), |
| | at the 2-(hydroxyimino)-3- | | 4.98-4.87 (m, 1.5H), |
| | methylbutoxy group.) | | 4.72-4.64 (m, 0.5H), |
| | | | 4.44 (dd, J = 10.5, 7.8 |
| | | | Hz, 0.5H), 4.37 (dd, J = |
| | | | 10.7, 7.7 Hz, 0.5H), 3.46- |
| | | | 3.40 (m, 0.3H), 2.88- |
| | | | 2.77 (m, 1.7H), 1.69- |
| | | | 1.64 (m, 3H), 1.24 (d, J = |
| | | | 7.1 Hz, 2H), 1.20 (dd, J = |
| | | | 7.7, 6.9 Hz, 4H), 0.87- |
| | | | 0.81 (m, 3H) ppm; |
| | | | amides NH and NH₂ not |
| | | | observed. |
| 187 | rel-4-((2R*,3S*,4S*,5R*)-3- | ESI-MS m/z calc. | ¹H NMR (500 MHz, |
| | (3,4-difluoro-2-(2- | 558.1902, found 559.7 | DMSO-d₆) δ 10.97 (s, |
| | (hydroxyimino)-3- | (M + 1)⁺; Retention time: | 1H), 10.69 (s, 1H), 8.47 |
| | methylbutoxy)phenyl)-4,5- | 3.42 minutes | (d, J = 5.5 Hz, 1H), 8.26 |
| | dimethyl-5- | | (d, J = 2.0 Hz, 1H), 8.04 |
| | (trifluoromethyl)tetrahydrofuran- | | (d, J = 2.7 Hz, 1H), 7.83- |
| | 2-carboxamido)picolinamide | | 7.79 (m, 1H), 7.59 (s, |
| | (First eluting isomer by SFC on | | 1H), 7.20 (dd, J = 8.4, 5.2 |
| | a Lux i-Cellulose-5 column, rt = | | Hz, 2H), 5.11 (d, J = 10.3 |
| | 5.92 min) | | Hz, 1H), 4.97-4.80 (m, |
| | | | 2H), 4.30 (dd, J = 10.3, |
| | | | 7.5 Hz, 1H), 2.83-2.70 |
| | | | (m, 2H), 1.57 (s, 3H), |
| | | | 1.13 (dd, J = 6.9, 4.8 Hz, |
| | | | 6H), 0.73 (d, J = 7.3 Hz, |
| | | | 3H) ppm. |
| 188 | rel-4-((2R*,3S*,4S*,5R*)-3- | ESI-MS m/z calc. | ¹H NMR (500 MHz, |
| | (3,4-difluoro-2-(2- | 558.1902, found 559.7 | DMSO-d₆) δ 11.06 (s, |
| | (hydroxyimino)-3- | (M + 1)⁺; Retention time: | 1H), 10.73 (s, 1H), 8.49 |
| | methylbutoxy)phenyl)-4,5- | 3.45 minutes | (d, J = 5.5 Hz, 1H), 8.27 |
| | dimethyl-5- | | (d, J = 2.1 Hz, 1H), 8.05 |
| | (trifluoromethyl)tetrahydrofuran- | | (s, 1H), 7.81 (dd, J = 5.6, |
| | 2-carboxamido)picolinamide | | 2.2 Hz, 1H), 7.61 (s, 1H), |
| | (Second eluting isomer by SFC | | 7.25-7.15 (m, 2H), 5.12 |
| | on a Lux i-Cellulose-5 column, | | (d, J = 10.3 Hz, 1H), 4.64 |
| | rt = 6.72 min) | | (dd, J = 59.9, 10.6 Hz, |
| | | | 2H), 4.27 (dd, J = 10.3, |
| | | | 7.5 Hz, 1H), 3.29-3.24 |
| | | | (m, 1H), 2.77 (q, J = 7.4 |
| | | | Hz, 1H), 1.56 (s, 3H), |
| | | | 1.16 (dd, J = 9.7, 7.0 Hz, |
| | | | 6H), 0.73 (d, J = 7.4 Hz, |
| | | | 3H) ppm. |
| 189 | 4-((2R,3S,4S,5R)-3-(3,4- | ESI-MS m/z calc. | ¹H NMR (500 MHz, |
| | difluoro-2-((Z)-2- | 572.2058, found 573.6 | Methanol-d₄) δ 8.48 (dd, |
| | (hydroxyimino)-3,3- | (M + 1)⁺; Retention time: | J = 5.5, 0.6 Hz, 1H), 8.26- |
| | dimethylbutoxy)phenyl)-4,5- | 3.63 minutes | 8.22 (m, 1H), 7.89 (dd, J = |
| | dimethyl-5- | | 5.5, 2.2 Hz, 1H), 7.17 |
| | (trifluoromethyl)tetrahydrofuran- | | (ddd, J = 8.0, 5.6, 2.1 Hz, |
| | 2-carboxamido)picolinamide | | 1H), 7.01 (td, J = 9.4, 7.6 |
| | | | Hz, 1H), 5.06 (d, J = 10.8 |
| | | | Hz, 1H), 4.79 (dd, J = |
| | | | 9.8, 0.9 Hz, masked 2H), |
| | | | 4.52 (dd, J = 10.8, 7.6 Hz, |
| | | | 1H), 2.82 (p, J = 7.6 Hz, |
| | | | 1H), 1.66 (s, 3H), 1.19 (s, |
| | | | 9H), 0.79 (dt, J = 7.6, 2.4 |
| | | | Hz, 3H) ppm; amides NH |
| | | | and NH₂ and alcohol OH |
| | | | not observed. |
| 190 | 4-((2R,3S,4S,5R)-3-(3,4- | ESI-MS m/z calc. | ¹H NMR (500 MHz, |
| | difluoro-2-(2-(1- | 574.1651, found 575.6 | Methanol-d₄) δ 8.48 (d, J = |
| | fluorocyclopropyl)-2- | (M + 1)⁺; Retention time: | 5.5 Hz, 1H), 8.24 (d, J = |
| | (hydroxyimino)ethoxy)phenyl)- | 3.31 minutes | 2.1 Hz, 1H), 7.89 (dd, J = |
| | 4,5-dimethyl-5- | | 5.5, 2.2 Hz, 1H), 7.17 |
| | (trifluoromethyl)tetrahydrofuran- | | (ddd, J = 8.0, 5.4, 2.0 Hz, |
| | 2-carboxamido)picolinamide | | 1H), 7.03 (td, J = 9.6, 7.8 |
| | (Mixture of geometric isomers | | Hz, 1H), 5.15-5.09 (m, |
| | at the 2-(1-fluorocyclopropyl)- | | 1H), 5.07 (d, J = 10.6 Hz, |
| | 2-(hydroxyimino)ethoxy | | 1H), 4.98 (d, J = 11.4 Hz, |
| | group.) | | 1H), 4.42 (dd, J = 10.6, |
| | | | 7.8 Hz, 1H), 2.82 (p, J = |
| | | | 7.6 Hz, 1H), 1.64 (s, 3H), |
| | | | 1.20-0.95 (m, 4H), 0.86- |
| | | | 0.76 (m, 3H) ppm; |
| | | | amides NH and NH₂ and |
| | | | alcohol OH not observed. |
| 191 | 4-((2R,3S,4S,5R)-3-(2-(2- | ESI-MS m/z calc. | ¹H NMR (500 MHz, |
| | cyclobutyl-2- | 570.1902, found 571.6 | Methanol-d₄) δ 8.48 (d, J = |
| | (hydroxyimino)ethoxy)-3,4- | (M + 1)⁺; Retention time: | 5.5 Hz, 1H), 8.25 (dd, J = |
| | difluorophenyl)-4,5-dimethyl-5- | 3.45 minutes | 5.0, 2.1 Hz, 1H), 7.90 |
| | (trifluoromethyl)tetrahydrofuran- | | (dd, J = 5.1, 2.8 Hz, 1H), |
| | 2-carboxamido)picolinamide | | 7.16 (dtd, J = 7.3, 5.3, 1.9 |
| | (Mixture of geometric isomers | | Hz, 1H), 7.06-6.98 (m, |
| | at the 2-cyclobutyl-2- | | 1H), 5.07 (dd, J = 10.5, |
| | (hydroxyimino)ethoxy group.) | | 4.7 Hz, 1H), 4.97-4.84 |
| | | | (m, 1H), 4.75 (qd, J = |
| | | | 10.8, 1.0 Hz, 1H), 4.39 |
| | | | (dd, J = 10.4, 7.9 Hz, |
| | | | 0.6H), 4.32-4.26 (m, |
| | | | 0.4H), 3.79-3.66 (m, |
| | | | 0.4H), 3.39 (p, J = 8.6 |
| | | | Hz, 0.6H), 2.80 (h, J = |
| | | | 7.6 Hz, 1H), 2.33-2.10 |
| | | | (m, 4H), 2.01-1.91 (m, |
| | | | 1H), 1.86-1.76 (m, 1H), |
| | | | 1.66 (d, J = 9.7 Hz, 3H), |
| | | | 0.81 (ddt, J = 11.9, 6.7, |
| | | | 2.4 Hz, 3H) ppm; amides |
| | | | NH and NH₂ and alcohol |
| | | | OH not observed. |

The following compound was made using the method described in Example 21, except that 2-bromo-5,8-dioxaspiro[3.4]octane was used as the alkylating agent in step 1. Between step 1 and 2, a ketal deprotection step was carried out at 55° C. for 12 h using 1 M HCl in EtOH as the solvent, conditions well known in the art. Step 3 was not carried out:

| Cmpd No. | Compound Name | LC/MS | NMR (shifts in ppm) |
|---|---|---|---|
| 192 | 4-((2R,3S,4S,5R)-3-(3,4- | ESI-MS m/z calc. | ¹H NMR (500 MHz, |
| | difluoro-2-(3- | 542.1589, found 543.6 | Methanol-d₄) δ 8.50 (d, |
| | (hydroxyimino)cyclobutoxy)phenyl)- | (M + 1)⁺; 541.5 (M − 1)⁻; | 1H), 8.25 (d, 1H), 7.95 |
| | 4,5-dimethyl-5- | Retention time: 3.02 | (m, 1H), 7.22-7.18 (m, |
| | (trifluoromethyl)tetrahydrofuran- | minutes | 1H), 7.08-6.98 (m, 1H), |
| | 2-carboxamido)picolinamide | | 5.12-5.04 (m, 2H), 4.41- |
| | (Mixture of geometric isomers | | 4.36 (m, 1H), 3.45-3.25 |
| | at the 3-(hydroxyimino) | | (masked, 2H), 3.17-3.03 |
| | cyclobutoxy group.) | | (m, 2H), 2.86-2.79 (m, |
| | | | 1H), 1.69 (s, 3H), 0.85- |
| | | | 0.83 (m, 3H) ppm; |
| | | | amides NH and NH₂ and |
| | | | alcohol OH not observed. |

The following compounds were made using the method described in Example 21, except that different alkylating agents were used in step 1. In step 2, a 25-30 wt % methoxylamine hydrochloride solution in water was used in place of hydroxylamine. In the case of compound 193, step 3 was not carried out. In the case of compound 194 and 195, the purification step 3 was performed by chiral SFC using a Chiralpak AS-H column, 5 μm particle size, 25 cm×10 mm from Daicel Corporation (Mobile phase: 18% methanol (supplemented with 20 mM NH₃), 82% CO₂; System pressure: 100 bar) on a Minigram SFC instrument from Berger Instruments:

| Cmpd No. | Compound Name | LC/MS | NMR (shifts in ppm) |
|---|---|---|---|
| 193 | 4-((2R,3S,4S,5R)-3-(3,4- | ESI-MS m/z calc. | ¹H NMR (500 MHz, |
| | difluoro-2-(2- | 543.17926, found 545.2 | Methanol-d₄) δ 8.48 (dt, J = |
| | (methoxyimino)propoxy)phenyl)- | (M + 1)⁺; 543.2 (M − 1)⁻; | 5.4, 0.8 Hz, 1H), 8.26 |
| | 4,5-dimethyl-5- | Retention time: 3.46 and | (t, J = 1.8 Hz, 1H), 7.90 |
| | (trifluoromethyl)tetrahydrofuran- | 3.47 minutes | (dt, J = 5.5, 2.1 Hz, 1H), |
| | 2-carboxamido)picolinamide | | 7.20-7.12 (m, 1H), 7.08- |
| | (Mixture of geometric isomers | | 6.98 (m, 1H), 5.08 (dd, J = |
| | at the 2-(methoxyimino) | | 10.5, 1.4 Hz, 1H), 5.00- |
| | propoxy group.) | | 4.94 (m, 0.5H), 4.86 (d, |
| | | | J = 14.3 Hz, 0.5H), 4.68- |
| | | | 4.60 (m, 1H), 4.34 (ddd, J = |
| | | | 10.5, 7.9, 4.1 Hz, 1H), |
| | | | 3.80 (s, 1.5H), 3.76 (s, |
| | | | 1.5H), 2.78 (pd, J = 7.6, |
| | | | 4.7 Hz, 1H), 2.07 (s, |
| | | | 1.5H), 1.99 (s, 1.5H), |
| | | | 1.65 (dd, J = 2.2, 1.1 Hz, |
| | | | 3H), 0.82 (tt, J = 7.8, 2.3 |
| | | | Hz, 3H) ppm; amides NH |
| | | | and NH₂ not observed. |
| 194 | rel-4-((2R*,3S*,4S*,5R*)-3- | ESI-MS m/z calc. | ¹H NMR (500 MHz, |
| | (3,4-difluoro-2-(2- | 544.1745, found 545.7 | Methanol-d₄) δ 8.49 (dd, |
| | (methoxyimino)propoxy)phenyl)- | (M + 1)⁺; 543.6 (M − 1)⁻; | J = 5.5, 0.6 Hz, 1H), 8.26 |
| | 4,5-dimethyl-5- | Retention time: 3.46 | (dd, J = 2.2, 0.6 Hz, 1H), |
| | (trifluoromethyl)tetrahydrofuran- | minutes | 7.90 (dd, J = 5.5, 2.2 Hz, |
| | 2-carboxamido)picolinamide | | 1H), 7.17 (ddd, J = 8.1, |
| | (First eluting isomer by SFC on | | 5.5, 2.2 Hz, 1H), 7.04 |
| | a Chiralpak AS-H column, rt = | | (ddd, J = 9.8, 8.9, 7.6 Hz, |
| | 4.04 min) | | 1H), 5.08 (d, J = 10.3 Hz, |
| | | | 1H), 5.00-4.84 (m, 2H), |
| | | | 4.35 (dd, J = 10.3, 8.0 Hz, |
| | | | 1H), 3.76 (s, 3H), 2.78 (p, |
| | | | J = 7.6 Hz, 1H), 2.07 (d, J = |
| | | | 0.8 Hz, 3H), 1.65 (d, J = |
| | | | 1.4 Hz, 3H), 0.83 (dq, J = |
| | | | 7.4, 2.3 Hz, 3H) ppm; |
| | | | amides NH and NH₂ not |
| | | | observed. |
| 195 | rel-4-((2R*,3S*,4S*,5R*)-3- | ESI-MS m/z calc. | ¹H NMR (500 MHz, |
| | (3,4-difluoro-2-(2- | 544.1745, found 545.7 | Methanol-d₄) δ 8.48 (dt, J = |
| | (methoxyimino)propoxy)phenyl)- | (M + 1)⁺; 543.6 (M − 1)⁻; | 5.4, 0.8 Hz, 1H), 8.26 |
| | 4,5-dimethyl-5- | Retention time: 3.49 | (t, J = 1.8 Hz, 1H), 7.90 |
| | (trifluoromethyl)tetrahydrofuran- | minutes | (dt, J = 5.5, 2.1 Hz, 1H), |
| | 2-carboxamido)picolinamide | | 7.20-7.12 (m, 1H), 7.08- |
| | (Second eluting isomer by SFC | | 6.98 (m, 1H), 5.08 (dd, J = |
| | on a Chiralpak AS-H column, rt = | | 10.5, 1.4 Hz, 1H), 5.00- |
| | 4.86 min) | | 4.94 (m, 0.5H), 4.86 (d, |
| | | | J = 14.3 Hz, 0.5H), 4.68- |
| | | | 4.60 (m, 1H), 4.34 (ddd, J = |
| | | | 10.5, 7.9, 4.1 Hz, 1H), |
| | | | 3.80 (s, 1.5H), 3.76 (s, |
| | | | 1.5H), 2.78 (pd, J = 7.6, |
| | | | 4.7 Hz, 1H), 2.07 (s, |
| | | | 1.5H), 1.99 (s, 1.5H), |
| | | | 1.65 (dd, J = 2.2, 1.1 Hz, |
| | | | 3H), 0.82 (tt, J = 7.8, 2.3 |
| | | | Hz, 3H) ppm; amides NH |
| | | | and NH₂ not observed. |

The following compound was made using the method described in Example 21, except that 4-((2R,3S,4S,5R)-3-(2-hydroxy-3-(trifluoromethyl)phenyl)-4,5-dimethyl-5-(trifluoromethyl)tetrahydrofuran-2-carboxamido) picolinamide (Product of Example 27, Step 11) was used as the starting material for step 1 and the reaction was carried out with 1-chloropropan-2-one as the alkylating agent. Step 3 was not carried out:

| Cmpd No. | Compound Name | LC/MS | NMR (shifts in ppm) |
|---|---|---|---|
| 196 | 4-((2R,3S,4S,5R)-3-(2-(2- | ESI-MS m/z calc. | ¹H NMR (400 MHz, |
| | (hydroxyimino)propoxy)-3- | 562.1651, found 563.5 | DMSO-d₆) δ 11.06 (s, |
| | (trifluoromethyl)phenyl)-4,5- | (M + 1)⁺; Retention time: | 0.5H), 10.58 (s, 0.5H), |
| | dimethyl-5- | 3.30 minutes | 8.51-8.46 (m, 0.5H), |
| | (trifluoromethyl)tetrahydrofuran- | | 8.38 (d, J = 5.5 Hz, |
| | 2-carboxamido)picolinamide | | 0.5H), 8.29 (d, J = 2.0 |
| | (Mixture of geometric isomers | | Hz, 0.5H), 8.22 (d, J = |
| | at the 2-(hydroxyimino) | | 2.2 Hz, 0.5H), 8.05 (s, |
| | propoxy group.) | | 0.5H), 7.97 (s, 0.5H), |
| | | | 7.82 (dd, J = 5.5, 2.2 Hz, |
| | | | 0.5H), 7.73 (d, J = 7.8 |
| | | | Hz, 0.5H), 7.67 (d, J = |
| | | | 7.8 Hz, 0.5H), 7.61 (s, |
| | | | 0.5H), 7.57 (dd, J = 5.6, |
| | | | 2.1 Hz, 0.5H), 7.51 (s, |
| | | | 0.5H), 7.40 (t, J = 7.9 Hz, |
| | | | 0.5H), 7.09 (d, J = 7.3 |
| | | | Hz, 0.5H), 7.03 (d, J = |
| | | | 7.4 Hz, 0.5H), 6.51 (s, |
| | | | 1H), 6.19 (t, J = 7.7 Hz, |
| | | | 0.5H), 5.77-5.69 (m, |
| | | | 0.5H), 5.17 (d, J = 10.2 |
| | | | Hz, 0.5H), 4.48 (d, J = |
| | | | 10.4 Hz, 0.5H), 4.44- |
| | | | 4.34 (m, 1H), 3.84-3.73 |
| | | | (m, 0.5H), 2.84 (t, J = 7.5 |
| | | | Hz, 0.5H), 2.61 (t, J = 7.3 |
| | | | Hz, 0.5H), 1.97 (s, 1.5H), |
| | | | 1.75 (s, 1.5H), 1.59 (s, |
| | | | 1.5H), 1.54 (s, 1.5H), |
| | | | 0.77 (d, J = 7.4 Hz, |
| | | | 1.5H), 0.72 (d, J = 7.6 |
| | | | Hz, 1.5H) ppm; 1H |
| | | | coincides with water |
| | | | signal. |

### Example 22

### rel-(2R*,3S*,4S*,5R*)-N-(3-carbamoylphenyl)-3-(3,4-difluoro-2-(((1r,3S)-3-hydroxycyclobutyl) methoxy)phenyl)-4,5-dimethyl-5-(trifluoromethyl)tetrahydrofuran-2-carboxamide (197) and rel-(2R*,3S*,4S*,5R*)—N-(3-carbamoylphenyl)-3-(3,4-difluoro-2-(((1s,3R)-3-hydroxycyclobutyl) methoxy)phenyl)-4,5-dimethyl-5-(trifluoromethyl)tetrahydrofuran-2-carboxamide (198)

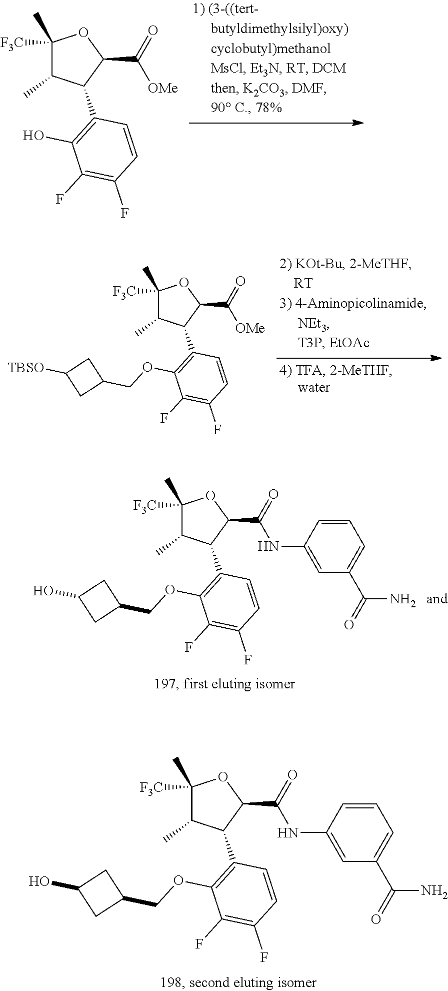

### Step 1:

MsCl (40 μL, 0.5168 mmol) was added to a stirred solution of (3-((tert-butyldimethylsilyl)oxy)cyclobutyl) methanol (100 mg, 0.462 mmol) and Et₃N (80 μL, 0.574 mmol) in DCM (3 mL) under a nitrogen atmosphere. The reaction mixture was stirred at ambient temperature for 4 h. The suspension was diluted with DCM and partitioned with a saturated aqueous NaHCO₃ solution. After stirring the mixture for 5 min, the organic phase was isolated by passing the mixture through a phase separation cartridge. The organic layer was concentrated in vacuo to give (3-((tert-butyldimethylsilyl)oxy)cyclobutyl)methyl methanesulfonate, which was used in the next step without any further purification.

K₂CO₃ (80 mg, 0.579 mmol) and methyl(2R,3S,4S,5R)-3-(3,4-difluoro-2-hydroxyphenyl)-4,5-dimethyl-5-(trifluoromethyl)tetrahydrofuran-2-carboxylate (Product of Example 20, Step 1, 100 mg, 0.282 mmol) were successively added to a stirred solution of (3-((tert-Butyldimethylsilyl)oxy)cyclobutyl)methyl methanesulfonate in DMF (2 mL). The reaction mixture was heated at 90° C. for 18 h under a nitrogen atmosphere. The reaction mixture was partitioned between EtOAc, a saturated aqueous sodium bicarbonate solution and brine. The organic phase was separated, dried over MgSO₄, filtered, and concentrated in vacuo. Purification by flash chromatography (12 g SiO₂, 0 to 100% EtOAc in hexanes) gave methyl(2R,3S,4S,5R)-3-(2-((3-((tert-butyldimethylsilyl)oxy)cyclobutyl) methoxy)-3,4-difluorophenyl)-4,5-dimethyl-5-(trifluoromethyl)tetrahydrofuran-2-carboxylate as a mixture of diastereomers at the (3-((tert-butyldimethylsilyl)oxy)cyclobutyl) methoxy group (200 mg, 78%), and as a light brown gum. ESI-MS m/z calc. 552.23303, found 553.3 (M+1)⁺; Retention time: 1.39 minutes.

### Step 2:

Potassium tert-butoxide (80 mg, 0.713 mmol) was added to a stirred solution of methyl(2R,3S,4S,5R)-3-(2-((3-((tert-butyldimethylsilyl)oxy)cyclobutyl) methoxy)-3,4-difluorophenyl)-4,5-dimethyl-5-(trifluoromethyl)tetrahydrofuran-2-carboxylate (200 mg, 0.362 mmol) in 2-MeTHF (5 mL). The solution was stirred at ambient temperature for 2 h. The reaction mixture was diluted with EtOAc and partitioned with 1M HCl. The organic layer was separated, washed with brine, dried over MgSO₄, filtered, and concentrated in vacuo to give (2R,3S,4S,5R)-3-(2-((3-((tert-butyldimethylsilyl)oxy)cyclobutyl) methoxy)-3,4-difluorophenyl)-4,5-dimethyl-5-(trifluoromethyl)tetrahydrofuran-2-carboxylic acid as a mixture of diastereomers at the (3-((tert-butyldimethylsilyl)oxy)cyclobutyl) methoxy group. ESI-MS m/z calc. 538.2174, found 539.3 (M+1)⁺; 537.3 (M−1); Retention time: 0.92 minutes.

### Step 3:

T3P (500 μL of 50% w/v, 0.786 mmol) was added to a stirred solution of (2R,3S,4S,5R)-3-(2-((3-((tert-butyldimethylsilyl)oxy)cyclobutyl) methoxy)-3,4-difluorophenyl)-4,5-dimethyl-5-(trifluoromethyl)tetrahydrofuran-2-carboxylic acid, 4-aminopyridine-2-carboxamide (80 mg, 0.583 mmol) and Et₃N (250 μL, 1.794 mmol) in EtOAc (3 mL). The reaction mixture was stirred at ambient temperature for 3 h. The mixture was diluted with EtOAc, washed with a saturated sodium bicarbonate solution and brine. The organic extracts were dried (MgSO₄), filtered and concentrated in vacuo to give 4-((2R,3S,4S,5R)-3-(2-((3-((tert-butyldimethylsilyl)oxy)cyclobutyl) methoxy)-3,4-difluorophenyl)-4,5-dimethyl-5-(trifluoromethyl)tetrahydrofuran-2-carboxamido) picolinamide, as a mixture of diastereomers at the (3-((tert-butyldimethylsilyl)oxy)cyclobutyl) methoxy group. ESI-MS m/z calc. 657.26575, found 658.8 (M+1)⁺; 656.7 (M−1); Retention time: 1.29 minutes.

### Step 4:

TFA (250 μL, 3.245 mmol) was added to a stirred solution of 4-((2R,3S,4S,5R)-3-(2-((3-((tert-butyldimethylsilyl)oxy)cyclobutyl) methoxy)-3,4-difluorophenyl)-4,5-dimethyl-5-(trifluoromethyl)tetrahydrofuran-2-carboxamido) picolinamide in 2-MeTHF (2 mL) and water (100 μL, 5.551 mmol). The reaction mixture was stirred at ambient temperature for 3 h. The reaction mixture was diluted with EtOAc, washed with a saturated aqueous sodium bicarbonate solution and brine. The organic extracts were dried over MgSO₄, filtered and concentrated in vacuo. Purification by reverse phase preparative HPLC gave two isomers of unknown absolute configuration:

First Eluting Isomer: rel-(2R*,3S*,4S*,5R*)—N-(3-carbamoylphenyl)-3-(3,4-difluoro-2-(((1r,3S)-3-hydroxycyclobutyl) methoxy)phenyl)-4,5-dimethyl-5-(trifluoromethyl)tetrahydrofuran-2-carboxamide (197, 5.72 mg, 3%). ¹H NMR (500 MHZ, Methanol-d₄) δ 8.44 (d, 1H), 8.21 (d, 1H), 7.85-7.83 (d, 1H), 7.12-7.04 (m, 1H), 6.98-6.94 (m, 1H), 5.03-5.01 (d, 1H), 4.33-4.31 (m, 1H), 4.13-4.10 (m, 1H), 4.09-3.98 (m, 2H), 2.78-2.74 (m, 1H), 2.40-2.32 (m, 2H), 2.12-2.15 (m, 1H), 1.78-1.72 (m, 2H), 1.62 (s, 3H), 0.84-0.82 (m, 3H) ppm; amides NH and NH₂ and alcohol OH not observed. ESI-MS m/z calc. 543.17926, found 544.6 (M+1)⁺; 542.6 (M−1); Retention time: 3.11 minutes.

Second Eluting Isomer: rel-2R*,3S*,4S*,5R*)—N-(3-carbamoylphenyl)-3-(3,4-difluoro-2-(((1s,3R)-3-hydroxycyclobutyl) methoxy)phenyl)-4,5-dimethyl-5-(trifluoromethyl)tetrahydrofuran-2-carboxamide (198, 1.3 mg, 1%). ¹H NMR (500 MHZ, Methanol-d₄) δ 8.13 (d, 1H), 7.38 (d, 1H), 7.34-7.29 (m, 1H), 7.04-6.98 (m, 1H), 6.76 (dd, 1H), 5.21-5.19 (d, 1H), 4.27-4.19 (m, 2H), 4.07-4.02 (qd, 2H), 2.84-2.81 (m, 1H), 2.40-2.32 (m, 2H), 1.79-1.60 (m, 6H), 0.84-0.82 (m, 3H) ppm; amides NH and NH₂ and alcohol OH not observed. ESI-MS m/z calc. 543.17926, found 544.6 (M+1)⁺; 542.6 (M−1); Retention time: 3.32 minutes.

The following compounds were made using the method described in Example 22, except that different alcohols were used in step 1. Step 4 was not required:

| Cmpd No. | Compound Name | LC/MS | NMR (shifts in ppm) |
|---|---|---|---|
| 199 | rel-4-((2R*,3S*,4S*,5R*)-3- | ESI-MS m/z calc. | |
| | (3,4-difluoro-2-(((1r,3S)-3- | 557.1949, found 558.3 | |
| | hydroxy-3- | (M + 1)⁺; Retention time: | |
| | methylcyclobutyl)methoxy)phenyl)- | 3.34 minutes | |
| | 4,5-dimethyl-5- | | |
| | (trifluoromethyl)tetrahydrofuran- | | |
| | 2-carboxamido)picolinamide | | |
| 200 | rel-4-((2R*,3S*,4S*,5R*)-3- | ESI-MS m/z calc. | ¹H NMR (500 MHz, |
| | (3,4-difluoro-2-(((1s,3R)-3- | 557.1949, found 558.6 | Methanol-d₄) δ 8.51 (d, |
| | hydroxy-3- | (M + 1)⁺; 556.6 (M − 1)−; | 1H), 8.29 (d, 1H), 7.92 |
| | methylcyclobutyl)methoxy)phenyl)- | Retention time: 3.37 | (m, 1H), 7.17-7.12 (m, |
| | 4,5-dimethyl-5- | minutes | 1H), 7.05-6.98 (m, 1H), |
| | (trifluoromethyl)tetrahydrofuran- | | 5.14-5.12 (m, 1H), 4.38- |
| | 2-carboxamido)picolinamide | | 4.32 (m, 1H), 4.26-4.22 |
| | | | (m, 1H), 4.14-4.11 (m, |
| | | | 1H), 2.85-2.75 (m, 1H), |
| | | | 2.36-2.30 (m, 1H), 2.21- |
| | | | 2.12 (m, 2H), 1.96-1.89 |
| | | | (m, 2H), 1.69 (s, 3H), |
| | | | 1.33 (s, 3H), 0.83 (m, 3H) |
| | | | ppm; amides NH and |
| | | | NH₂ and alcohol OH not |
| | | | observed. |

The following compound was made using the method described in Example 22, except that, in step 1, rac-((1r,3r)-3-bromocyclobutoxy)(tert-butyl)dimethylsilane was used in place of the mesylate. 4-Amino-N-methylpicolinamide was used as the coupling partner in the amide coupling step 3:

| Cmpd No. | Compound Name | LC/MS | NMR (shifts in ppm) |
|---|---|---|---|
| 201 | 4-((2R,3S,4S,5R)-3-(3,4- | ESI-MS m/z calc. | ¹H NMR (500 MHz, |
| | difluoro-2-((1s,3R)-3- | 543.17926, found 544.6 | Methanol-d₄) δ 8.47 (d, J = |
| | hydroxycyclobutoxy)phenyl)- | (M + 1)⁺; 542.6 (M − 1)⁻; | 5.5 Hz, 1H), 8.23 (d, J = |
| | 4,5-dimethyl-5- | Retention time: 3.11 | 2.2 Hz, 1H), 7.88 (dd, J = |
| | (trifluoromethyl)tetrahydrofuran- | minutes | 5.5, 2.2 Hz, 1H), 7.15- |
| | 2-carboxamido)-N- | | 7.08 (m, 1H), 6.97 (dt, J = |
| | methylpicolinamide | | 9.9, 8.2 Hz, 1H), 5.07 |
| | | | (d, J = 10.6 Hz, 1H), 4.34 |
| | | | (td, J = 9.8, 8.5, 6.9 Hz, |
| | | | 2H), 3.85 (p, J = 7.2 Hz, |
| | | | 1H), 2.95 (s, 3H), 2.81 |
| | | | (ddp, J = 23.8, 12.2, 6.2 |
| | | | Hz, 3H), 2.15 (ddt, J = |
| | | | 27.0, 11.3, 7.5 Hz, 2H), |
| | | | 1.68 (s, 3H), 0.81 (dt, J = |
| | | | 7.4, 2.4 Hz, 3H) ppm; |
| | | | amides NH and alcohol |
| | | | OH not observed. |

The following compound was made using the method described in Example 22, except that, in step 1, rac-((1r,3r)-3-bromocyclobutoxy)(tert-butyl)dimethylsilane was used in place of the mesylate. 5-Amino-2-fluorobenzamide was used as the coupling partner in the amide coupling step 3:

| | | | |
|---|---|---|---|
| Cmpd No. | Compound Name | LC/MS | NMR (shifts in ppm) |

| | | |
|---|---|---|
| 202 | (2R,3S,4S,5R)-N-(3-carbamoyl- | ESI-MS m/z calc. |
| | 4-fluorophenyl)-3-(3,4- | 546.15894 , found 547.6 |
| | difluoro-2-((1s,3R)-3- | (M + 1)⁺; 545.6 (M − 1)⁻; |
| | hydroxycyclobutoxy)phenyl)- | Retention time: 3.06 |
| | 4,5-dimethyl-5- | minutes |
| | (trifluoromethyl)tetrahydrofuran- | |
| | 2-carboxamide | |

The following compound was made using the method described in Example 22, except that, in step 1, rac-((1r,3r)-3-bromocyclobutoxy)(tert-butyl)dimethylsilane was used in place of the mesylate. Methyl 4-amino-5-methylpicolinate (Intermediate N) was used as the coupling partner in the amide coupling step 3. At the end of the synthesis, the ester was further reacted using the conditions described in Example 2 step 9:

| Cmpd No. | Compound Name | LC/MS | NMR (shifts in ppm) |
|---|---|---|---|
| 203 | 4-((2R,3S,4S,5R)-3-(3,4- | ESI-MS m/z calc. | ¹H NMR (500 MHz, |
| | difluoro-2-((1s,3R)-3- | 543.17926, found 544.6 | Methanol-d₄) δ 8.48 (s, |
| | hydroxycyclobutoxy)phenyl)- | (M + 1)⁺; 542.6 (M − 1)⁻; | 1H), 8.44 (s, 1H), 7.21 |
| | 4,5-dimethyl-5- | Retention time: 3.11 | (ddd, J = 8.3, 5.5, 2.1 Hz, |
| | (trifluoromethyl)tetrahydrofuran- | minutes | 1H), 7.03-6.94 (m, 1H), |
| | 2-carboxamido)-5- | | 5.20 (d, J = 11.0 Hz, 1H), |
| | methylpicolinamide | | 4.33 (qd, J = 7.9, 7.3, 2.7 |
| | | | Hz, 2H), 3.85 (p, J = 7.1 |
| | | | Hz, 1H), 2.89-2.79 (m, |
| | | | 2H), 2.75 (ddd, J = 16.3, |
| | | | 10.9, 6.7 Hz, 1H), 2.29 (s, |
| | | | 3H), 2.16 (dt, J = 11.8, |
| | | | 7.5 Hz, 1H), 2.09 (dt, J = |
| | | | 11.5, 7.5 Hz, 1H), 1.72 (s, |
| | | | 3H), 0.83 (dd, J = 7.6, 2.4 |
| | | | Hz, 3H) ppm; amides NH |
| | | | and NH₂ and alcohol OH |
| | | | not observed. |

The following compound was made using the method described in Example 22, except that 6-((tert-butyldimethylsilyl)oxy)spiro[3.3]heptan-2-ol (Intermediate V) was used as the alcohol in step 1:

| | | | |
|---|---|---|---|
| Cmpd No. | Compound Name | LC/MS | NMR (shifts in ppm) |

| | | |
|---|---|---|
| 204 | 4-((2R,3S,4S,5R)-3-(3,4- | ESI-MS m/z calc. |
| | difluoro-2-((6- | 569.1949, found 570.6 |
| | hydroxyspiro[3.3]heptan-2- | (M + 1)⁺; 568.6 (M − 1)⁻; |
| | yl)oxy)phenyl)-4,5-dimethyl-5- | Retention time: 3.24 |
| | (trifluoromethyl)tetrahydrofuran- | minutes |
| | 2-carboxamido)picolinamide | |
| | (Mixture of diastereomers at the | |
| | (6-hydroxyspiro[3.3]heptan-2- | |
| | yl)oxy group.) | |

### Example 23

### 4-((2R,3S,4S,5R)-3-(4-(benzyloxy)-3-fluoro-2-methoxyphenyl)-4,5-dimethyl-5-(trifluoromethyl)tetrahydrofuran-2-carboxamido) picolinamide (205), 4-((2R,3S,4S,5R)-3-(3-fluoro-4-hydroxy-2-methoxyphenyl)-4,5-dimethyl-5-(trifluoromethyl)tetrahydrofuran-2-carboxamido) picolinamide (206) and 4-((2R,3S,4S,5R)-3-(4-(3,3-difluorocyclobutoxy)-3-fluoro-2-methoxyphenyl)-4,5-dimethyl-5-(trifluoromethyl)tetrahydrofuran-2-carboxamido) picolinamide (207)

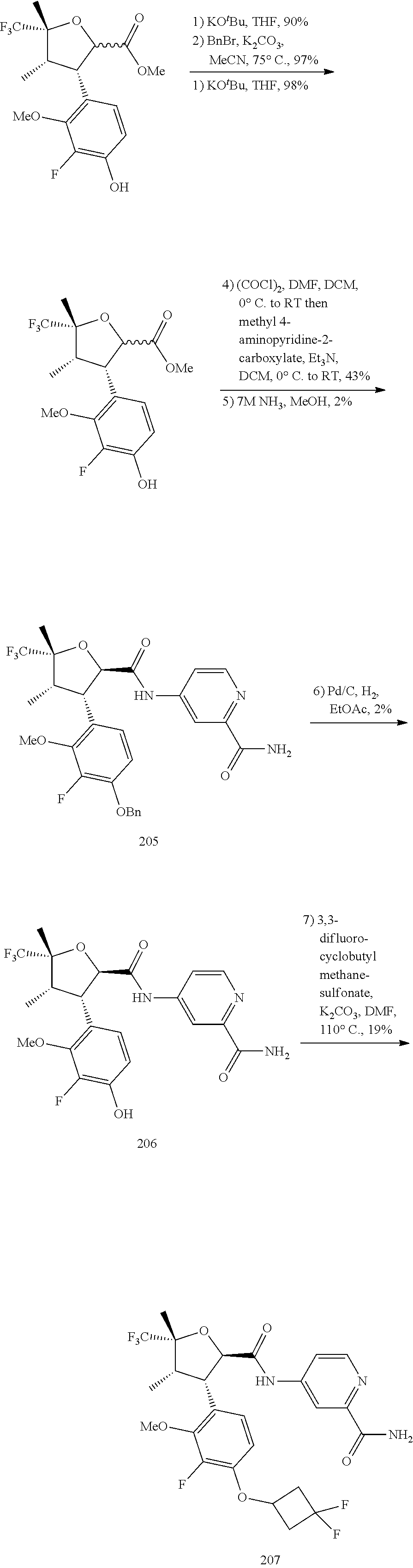 [SMILES: COC(=O)C1O[C@@](C)(C(F)(F)F)[C@@H](C)[C@H]1c1ccc(O)c(F)c1OC ; COC(=O)C1O[C@@](C)(C(F)(F)F)[C@@H](C)[C@H]1c1ccc(O)c(F)c1OC ; COc1c([C@H]2[C@H](C(=O)Nc3ccnc(C(N)=O)c3)O[C@@](C)(C(F)(F)F)[C@H]2C)ccc(OCc2ccccc2)c1F ; COc1c([C@H]2[C@H](C(=O)Nc3ccnc(C(N)=O)c3)O[C@@](C)(C(F)(F)F)[C@H]2C)ccc(O)c1F ; COc1c([C@H]2[C@H](C(=O)Nc3ccnc(C(N)=O)c3)O[C@@](C)(C(F)(F)F)[C@H]2C)ccc(OC2CC(F)(F)C2)c1F]

### Step 1:

Potassium tert-butoxide (400 mg, 3.565 mmol) was added to a stirred solution of the methyl(3S,4S,5R)-3-(3-fluoro-4-hydroxy-2-methoxyphenyl)-4,5-dimethyl-5-(trifluoromethyl)tetrahydrofuran-2-carboxylate (Product of Example 19, Step 6, 300 mg, 0.819 mmol) in THF (5 mL) at ambient temperature. The reaction mixture was stirred at ambient temperature for 5 min. The mixture was quenched by addition of a saturated ammonium chloride solution (5 mL). The mixture was diluted with DCM (5 mL). The aqueous phase was separated and extracted with DCM (5 mL). The aqueous phase was acidified to pH 0 with 1N HCl and extracted with DCM (2×10 mL). The combined organic extracts were dried over MgSO₄, filtered, and concentrated in vacuo to give (2R,3S,4S,5R)-3-(3-fluoro-4-hydroxy-2-methoxyphenyl)-4,5-dimethyl-5-(trifluoromethyl)tetrahydrofuran-2-carboxylic acid (260 mg, 90%) which was used in the next step without further purification. ESI-MS m/z calc. 352.09338, found 351.4 (M−1); Retention time: 0.5 minutes.

### Step 2:

BnBr (70 μL, 0.589 mmol) was added to a mixture of (2R,3S,4S,5R)-3-(3-fluoro-4-hydroxy-2-methoxyphenyl)-4,5-dimethyl-5-(trifluoromethyl)tetrahydrofuran-2-carboxylic acid (75 mg, 0.213 mmol) and K₂CO₃ (100 mg, 0.724 mmol) in MeCN (1 mL). The vial was sealed and the mixture was heated at 75° C. for 1 h. The mixture was quenched by addition of water (10 mL) and extracted with MTBE (2×10 mL). The combined organic extracts were dried over MgSO₄, filtered, and concentrated in vacuo. Purification by flash chromatography (4 g SiO₂, 0 to 100% AcOEt in heptane) gave benzyl(2R,3S,4S,5R)-3-(4-(benzyloxy)-3-fluoro-2-methoxyphenyl)-4,5-dimethyl-5-(trifluoromethyl)tetrahydrofuran-2-carboxylate (110 mg, 97%). ESI-MS m/z calc. 532.1873, found 533.7 (M+1)⁺; 531.7 (M−1); Retention time: 1.22 minutes.

### Step 3:

Potassium tert-butoxide (70 mg, 0.624 mmol) was added in one portion to a stirred solution of benzyl(2R,3S,4S,5R)-3-(4-(benzyloxy)-3-fluoro-2-methoxyphenyl)-4,5-dimethyl-5-(trifluoromethyl)tetrahydrofuran-2-carboxylate (110 mg, 0.207 mmol) in THF (2 mL) at ambient temperature. Immediate conversion was observed. The reaction mixture was quenched by addition of water (10 mL) and extracted with DCM (2×10 mL). The organic extracts were dried over MgSO₄, filtered, and concentrated in vacuo to give (2R,3S,4S,5R)-3-(4-(benzyloxy)-3-fluoro-2-methoxyphenyl)-4,5-dimethyl-5-(trifluoromethyl)tetrahydrofuran-2-carboxylic acid (90 mg, 98%), which was used in the next step without further purification. ESI-MS m/z calc. 442.14035, found 441.5 (M−1); Retention time: 0.71 minutes.

### Step 4, 5 and 6:

Oxalyl chloride (50 μL, 0.573 mmol) was added dropwise to a stirred solution of (2R,3S,4S,5R)-3-(4-(benzyloxy)-3-fluoro-2-methoxyphenyl)-4,5-dimethyl-5-(trifluoromethyl)tetrahydrofuran-2-carboxylic acid (90 mg, 0.203 mmol) and DMF (22.269 μL, 0.288 mmol) in DCM (1 mL) at ambient temperature. The reaction mixture was stirred at ambient temperature for 30 min. Upon completion of the reaction, the acid chloride was concentrated in vacuo. The residue, dissolved in DCM (500 μL), was added to a stirred solution of methyl 4-aminopyridine-2-carboxylate (50 mg, 0.329 mmol) and triethylamine (50 μL, 0.359 mmol) in DCM (500 μL) at ambient temperature. The reaction mixture was stirred at ambient temperature for 2 h. The mixture was quenched by addition of methanol (100 μL) and concentrated in vacuo. Purification by flash chromatography (4 g SiO₂, 0 to 100% AcOEt in heptane) gave methyl 4-((2R,3S,4S,5R)-3-(4-(benzyloxy)-3-fluoro-2-methoxyphenyl)-4,5-dimethyl-5-(trifluoromethyl)tetrahydrofuran-2-carboxamido) picolinate (50 mg, 43%), which was used in the next step without further purification. ESI-MS m/z calc. 576.18835, found 577.7 (M+1)⁺; 575.7 (M−1); Retention time: 1.07 minutes.

Methanolic ammonia (7 mL of 7 M, 49.00 mmol) was added to methyl 4-((2R,3S,4S,5R)-3-(4-(benzyloxy)-3-fluoro-2-methoxyphenyl)-4,5-dimethyl-5-(trifluoromethyl)tetrahydrofuran-2-carboxamido) picolinate and the mixture was stirred at ambient temperature until completion of the reaction. Purification by flash chromatography (4 g SiO₂, 0 to 100% AcOEt in heptane) gave 4-((2R,3S,4S,5R)-3-(4-(benzyloxy)-3-fluoro-2-methoxyphenyl)-4,5-dimethyl-5-(trifluoromethyl)tetrahydrofuran-2-carboxamido) picolinamide (205, 2.2 mg, 2%). ESI-MS m/z calc. 561.18866, found 562.7 (M+1)⁺; 560.6 (M−1); Retention time: 3.63 minutes.

A solution of 4-((2R,3S,4S,5R)-3-(4-(benzyloxy)-3-fluoro-2-methoxyphenyl)-4,5-dimethyl-5-(trifluoromethyl)tetrahydrofuran-2-carboxamido) picolinamide in ethyl acetate (3 mL) was added to a flask containing Pd/C (60 mg, 0.028 mmol). The mixture was stirred under an atmospheric pressure of hydrogen until complete conversion. The mixture was degassed with a stream of nitrogen. The catalyst was filtered through a pad of Celite®, and washed with DCM. The filtrate was concentrated in vacuo to give 4-((2R,3S,4S,5R)-3-(3-fluoro-4-hydroxy-2-methoxyphenyl)-4,5-dimethyl-5-(trifluoromethyl)tetrahydrofuran-2-carboxamido) picolinamide (206, 1.8 mg, 2%). ESI-MS m/z calc. 471.14172, found 472.6 (M+1)⁺; 470.5 (M−1); Retention time: 2.74 minutes.

### Step 7:

3,3-Difluorocyclobutyl methanesulfonate (Intermediate T-1) (8 mg, 0.04297 mmol) was added to a stirred solution of 4-((2R,3S,4S,5R)-3-(3-fluoro-4-hydroxy-2-methoxyphenyl)-4,5-dimethyl-5-(trifluoromethyl)tetrahydrofuran-2-carboxamido) picolinamide (15 mg, 0.028 mmol) and K₂CO₃ (12 mg, 0.087 mmol) in DMF (0.5 mL). The reaction mixture was heated to 110° C. in a sealed vial until completion of the reaction. The mixture was cooled to ambient temperature and quenched by addition of water (10 mL). The mixture was partitioned with DCM (10 mL). The aqueous phase was separated and extracted with DCM (10 mL). The combined organic extracts were dried over MgSO₄, filtered, and concentrated in vacuo. Purification by flash chromatography (4 g SiO₂, 0 to 100% AcOEt in heptane) gave 4-((2R,3S,4S,5R)-3-(4-(3,3-difluorocyclobutoxy)-3-fluoro-2-methoxyphenyl)-4,5-dimethyl-5-(trifluoromethyl)tetrahydrofuran-2-carboxamido) picolinamide (207, 3 mg, 19%). ESI-MS m/z calc. 561.16986, found 562.6 (M+1)⁺; 560.6 (M−1); Retention time: 3.51 minutes.

### Example 24

### 4-((2R,3S,4S,5R)-3-(6-(difluoromethyl)-2-(2-hydroxyethoxy)pyridin-3-yl)-4,5-dimethyl-5-(trifluoromethyl)tetrahydrofuran-2-carboxamido) picolinamide (208)

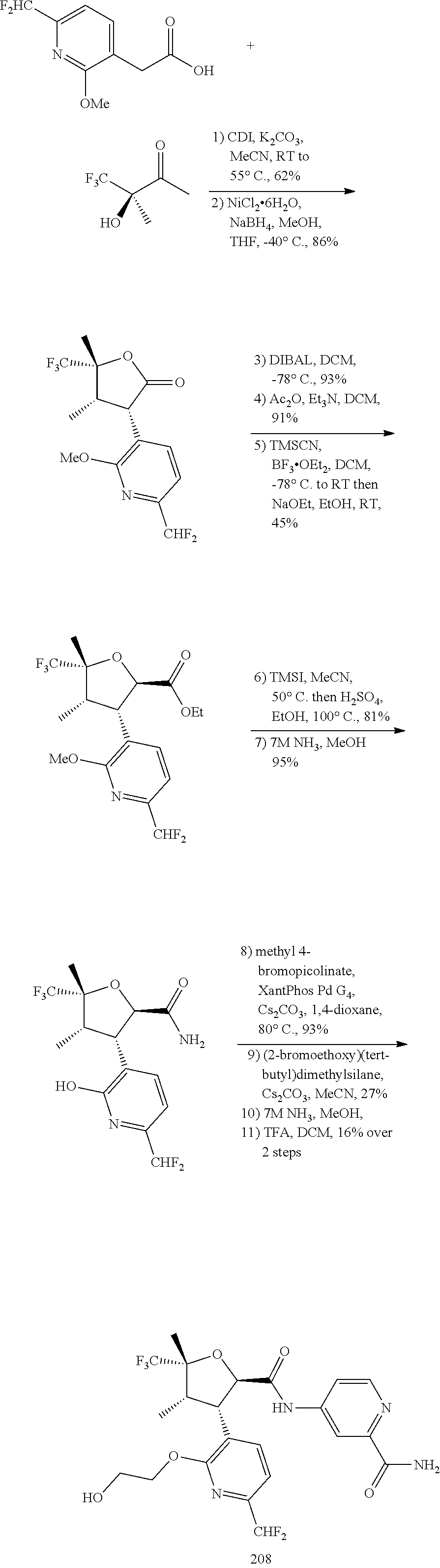

### Step 1:

2-(6-(Difluoromethyl)-2-methoxypyridin-3-yl) acetic acid (Intermediate D, 8.96 g, 38.267 mmol) was slowly added to a solution of carbonyl diimidazole (7.6 g, 46.870 mmol) in acetonitrile (50 mL). The reaction mixture was stirred at 40° C. for 45 min. To this clear, yellow solution was added (R)-4,4,4-trifluoro-3-hydroxy-3-methylbutan-2-one (Intermediate C, 7.84 g, 50.223 mmol) and potassium carbonate (7.05 g, 51.011 mmol). The reaction mixture was stirred at 55° C. for 2 h. The reaction was cooled to ambient temperature and diluted with MTBE (30 mL). The organic layer was separated, washed with 2 M hydrochoric acid (2×25 ml) and water (25 mL), dried over MgSO₄, filtered, and concentrated in vacuo to give an oil. Purification by flash chromatography (SiO₂, 10% ethyl acetate in heptane) gave (R)-3-(6-(difluoromethyl)-2-methoxypyridin-3-yl)-4,5-dimethyl-5-(trifluoromethyl) furan-2 (5H)-one (8.51 g, 62%) as a pale yellow oil. ESI-MS m/z calc. 337.0737, found 335.98 (M−1); Retention time: 2.45 minutes.

### Step 2:

Nickel dichloride hexahydrate (1.45 g, 6.100 mmol) was added to a stirred and previously degassed solution of (R)-3-(6-(difluoromethyl)-2-methoxypyridin-3-yl)-4,5-dimethyl-5-(trifluoromethyl) furan-2 (5H)-one (2.075 g, 6.153 mmol) in MeOH (50 mL) and THF (10 mL) at −40° C. NaBH₄ (1.17 g, 30.93 mmol) was added portionwise and the reaction mixture was stirred at −40° C. for 30 min. Further amounts of NiCl₂ (3×1 eq) and NaBH₄ (3×5 eq) were added portionwise. Upon completion of the reaction, the mixture was quenched by addition of a NH₄Cl solution and 2 M HCl. The aqueous phase was separated and extracted twice with EtOAc. The combined organic extracts were washed with water (2×) and brine, dried over MgSO₄, filtered, and concentrated in vacuo to give a mixture of stereoisomers with (3S,4S,5R)-3-(6-(difluoromethyl)-2-methoxypyridin-3-yl)-4,5-dimethyl-5-(trifluoromethyl)dihydrofuran-2 (3H)-one (1.79 g, 86%) as the main diastereoisomer, which was a colourless oil. ¹H NMR (500 MHZ, Chloroform-d) δ 7.74 (d, J=7.6 Hz, 1H), 7.25 (d, J=7.6 Hz, 1H), 6.52 (t, J=55.5 Hz, 1H), 4.51 (d, J=9.2 Hz, 1H), 4.00 (s, 3H), 3.02 (dq, J=9.3, 7.5 Hz, 1H), 1.73 (t, J=1.2 Hz, 3H), 0.78 (dq, J=7.5, 2.4 Hz, 3H) ppm. ESI-MS m/z calc. 339.0894, found 340.1 (M+1)⁺; 338.2 (M−1); Retention time: 0.97 minutes.

### Step 3:

DIBAL (7 mL of a 1 M solution in DCM, 7.0 mmol) was added dropwise to a stirred solution of (3S,4S,5R)-3-(6-(difluoromethyl)-2-methoxypyridin-3-yl)-4,5-dimethyl-5-(trifluoromethyl)dihydrofuran-2 (3H)-one (1.79 g, 5.276 mmol) in DCM (35 mL) at −78° C. The reaction mixture was stirred at ambient temperature for 1.5 h. A further amount of DIBAL (3.2 ml of a 1M solution in DCM) was added to the reaction, which was stirred at −78° C. for another 30 min. Upon complete conversion, the mixture was quenched by addition of a saturated ammonium chloride solution and a Rochelle's salt solution (30% w/w) (3 mL each). The mixture was diluted with DCM. The aqueous phase was separated and extracted with DCM (2×20 mL). The combined organic extracts were dried (MgSO₄), filtered and concentrated in vacuo to give a mixture of stereoisomers with (3S,4S,5R)-3-(6-(difluoromethyl)-2-methoxypyridin-3-yl)-4,5-dimethyl-5-(trifluoromethyl)tetrahydrofuran-2-ol (1.67 g, 93%) as the main stereoisomer. ¹H NMR (500 MHZ, Chloroform-d) δ 7.59 (d, J=7.6 Hz, 1H), 7.19 (d, J=7.6 Hz, 1H), 6.51 (t, J=55.7 Hz, 1H), 5.82 (t, J=3.9 Hz, 1H), 3.97 (s, 3H), 3.85-3.81 (m, 1H), 3.07-2.96 (m, 2H), 1.64 (d, J=1.3 Hz, 3H), 0.83 (dq, J=7.6, 2.2 Hz, 3H) ppm. ESI-MS m/z calc. 341.10504, found 342.5 (M+1)⁺; 340.5 (M−1); Retention time: 0.9 minutes.

### Step 4:

Ac2O (1.4 mL, 14.84 mmol) was added to a solution of (3S,4S,5R)-3-(6-(difluoromethyl)-2-methoxypyridin-3-yl)-4,5-dimethyl-5-(trifluoromethyl)tetrahydrofuran-2-ol (1.67 g, 4.893 mmol) and triethylamine (2.7 mL, 19.37 mmol) in DCM (50 mL) and the reaction was stirred at ambient temperature overnight. The mixture was quenched by addition of a NaHCO₃ solution and diluted with DCM. The organic phase was separated, washed with a NaHCO₃ solution, water and brine, dried over MgSO₄, filtered, and concentrated in vacuo to give (3S,4S,5R)-3-(6-(difluoromethyl)-2-methoxypyridin-3-yl)-4,5-dimethyl-5-(trifluoromethyl)tetrahydrofuran-2-yl acetate (1.71 g, 91%) as a colourless oil. ¹H NMR (500 MHz, Chloroform-d) δ 7.62 (dd, J=7.5, 1.7 Hz, 1H), 7.20 (d, J=7.6 Hz, 1H), 6.63-6.38 (m, 2H), 3.97 (s, 4H), 2.99 (p, J=7.8 Hz, 1H), 2.09 (s, 3H), 1.61 (d, J=1.2 Hz, 3H), 0.88 (dt, J=7.4, 2.1 Hz, 3H) ppm. ESI-MS m/z calc. 383.1156, found 385.0 (M+1)⁺; Retention time: 1.04 minutes.

### Step 5:

TMSCN (1.6 mL, 12.79 mmol) and BF₃·OEt₂ (1.4 mL, 11.34 mmol) were successively added to a stirred solution of (3S,4S,5R)-3-(6-(difluoromethyl)-2-methoxypyridin-3-yl)-4,5-dimethyl-5-(trifluoromethyl)tetrahydrofuran-2-yl acetate (1.7 g, 4.435 mmol) in DCM (50 mL) at −78° C. The reaction mixture was stirred at −78° C. for 1 h and at ambient temperature for 30 min. The mixture was quenched with a 1 M sodium carbonate solution (5 mL) and diluted with water. The aqueous phase was separated and extracted with DCM (3×20 mL). The combined organic extracts were dried (MgSO₄), filtered and concentrated in vacuo to give (3S,4S,5R)-3-(6-(difluoromethyl)-2-methoxypyridin-3-yl)-4,5-dimethyl-5-(trifluoromethyl)tetrahydrofuran-2-carbonitrile, which was directly used as is in the next step.

Sodium ethoxide (954 mg, 14.02 mmol) was added to a solution of (3S,4S,5R)-3-(6-(difluoromethyl)-2-methoxypyridin-3-yl)-4,5-dimethyl-5-(trifluoromethyl)tetrahydrofuran-2-carbonitrile in EtOH (25 mL) under a nitrogen atmosphere. The reaction mixture was stirred at ambient temperature overnight. The mixture was quenched by addition of a saturated NH₄Cl solution and diluted with ethyl acetate and water. The aqueous layer was extracted with EtOAc (2×). The combined organic extracts were dried (MgSO₄), filtered and concentrated in vacuo. Purification by flash chromatography (SiO₂, 0 to 50% EtOAc in heptane) gave ethyl(2R,3S,4S,5R)-3-(6-(difluoromethyl)-2-methoxypyridin-3-yl)-4,5-dimethyl-5-(trifluoromethyl)tetrahydrofuran-2-carboxylate (795 mg, 45%) as a yellow oil. ¹H NMR (500 MHz, Chloroform-d) δ 7.70 (d, J=7.6 Hz, 1H), 7.20 (d, J=7.6 Hz, 1H), 6.52 (t, J=55.6 Hz, 1H), 4.87 (d, J=10.7 Hz, 1H), 4.18-4.05 (m, 2H), 3.98 (s, 3H), 3.87 (dd, J=10.7, 7.9 Hz, 1H), 2.81 (p, J=7.6 Hz, 1H), 1.62 (s, 3H), 1.07 (t, J=7.1 Hz, 3H), 0.73 (dq, J=7.4, 2.4 Hz, 3H) ppm. ESI-MS m/z calc. 397.13126, found 398.2 (M+1)⁺; Retention time: 1.1 minutes.

### Step 6:

Iodotrimethylsilane (350 μL, 2.459 mmol) was added to an ice-cold solution of ethyl(2R,3S,4S,5R)-3-(6-(difluoromethyl)-2-methoxypyridin-3-yl)-4,5-dimethyl-5-(trifluoromethyl)tetrahydrofuran-2-carboxylate (750 mg, 1.888 mmol) in MeCN (15 mL). The reaction mixture was stirred for 1 h at 0° C. and warmed up to ambient temperature. Two further amounts of TMSI (350 ul) were added and the reaction was heated at 80° C. overnight. The mixture was concentrated in vacuo to give (2R,3S,4S,5R)-3-(6-(difluoromethyl)-2-hydroxypyridin-3-yl)-4,5-dimethyl-5-(trifluoromethyl)tetrahydrofuran-2-carboxylic acid. ESI-MS m/z calc. 355.0843, found 354.9 (M+1)⁺; 353.2 (M−1); Retention time: 0.59 minutes.

H₂SO₄ (50 μL, 0.938 mmol) was added to (2R,3S,4S,5R)-3-(6-(difluoromethyl)-2-hydroxypyridin-3-yl)-4,5-dimethyl-5-(trifluoromethyl)tetrahydrofuran-2-carboxylic acid in EtOH (20 mL). The reaction mixture was heated under reflux overnight. The mixture was then concentrated in vacuo. Purification by flash chromatography (SiO₂, 0 to 100% EtOAc in heptane) gave ethyl (2R,3S,4S,5R)-3-(6-(difluoromethyl)-2-hydroxypyridin-3-yl)-4,5-dimethyl-5-(trifluoromethyl)tetrahydrofuran-2-carboxylate (585 mg, 81%). ¹H NMR (500 MHZ, Chloroform-d) & 7.48 (d, J=7.1 Hz, 1H), 6.50 (dt, J=7.1, 1.5 Hz, 1H), 6.48 (t, J=54.4 Hz, 1H), 4.86 (d, J=9.6 Hz, 1H), 4.24-4.14 (m, 2H), 4.10-4.00 (m, 1H), 3.02 (p, J=7.6 Hz, 1H), 1.64 (d, J=1.4 Hz, 3H), 1.24 (t, J=7.1 Hz, 3H), 0.85 (dt, J=7.4, 2.3 Hz, 3H) ppm; alcohol OH not observed. ESI-MS m/z calc. 383.1156, found 384.9 (M+1)⁺; 382.3 (M−1); Retention time: 0.77 minutes.

### Step 7:

Ethyl(2R,3S,4S,5R)-3-(6-(difluoromethyl)-2-hydroxypyridin-3-yl)-4,5-dimethyl-5-(trifluoromethyl)tetrahydrofuran-2-carboxylate (97 mg, 0.253 mmol) was dissolved in methanolic ammonia (3 mL of 7 M solution in MeOH, 21.00 mmol) and the reaction mixture was stirred at 50° C. overnight. The mixture was cooled to ambient temperature and concentrated in vacuo to give (2R,3S,4S,5R)-3-(6-(difluoromethyl)-2-hydroxypyridin-3-yl)-4,5-dimethyl-5-(trifluoromethyl)tetrahydrofuran-2-carboxamide (85 mg, 95%). ESI-MS m/z calc. 354.10028, found 354.9 (M+1)⁺; 535.1 (M−1); Retention time: 0.59 minutes.

### Step 8:

A solution of (2R,3S,4S,5R)-3-(6-(difluoromethyl)-2-hydroxypyridin-3-yl)-4,5-dimethyl-5-(trifluoromethyl)tetrahydrofuran-2-carboxamide (105 mg, 0.296 mmol) in 1,4-dioxane (3 mL) was added to a mixture of methyl 4-bromopicolinate (105 mg, 0.486 mmol), XantPhos Pd G4 (26.2 mg, 0.027 mmol) and Cs₂CO₃ (293 mg, 0.899 mmol). The reaction was degassed by bubbling nitrogen gas through and heated at 80° C. for 3 h. The mixture was partitioned between water and EtOAc. The aqueous layer was separated and extracted with EtOAc (3×). The combined organic layers were dried over MgSO₄, filtered, and concentrated in vacuo. Purification by flash chromatography (SiO₂, 0 to 100% EtOAc in heptane) gave methyl 4-((2R,3S,4S,5R)-3-(6-(difluoromethyl)-2-hydroxypyridin-3-yl)-4,5-dimethyl-5-(trifluoromethyl)tetrahydrofuran-2-carboxamido) picolinate (135 mg, 93%). ¹H NMR (500 MHz, DMSO-d₆) δ 10.65 (s, 1H), 8.57 (d, J=5.5 Hz, 1H), 8.38 (d, J=2.1 Hz, 1H), 7.87 (dd, J=5.5, 2.1 Hz, 1H), 7.55 (s, 1H), 6.79 (t, J=54.3 Hz, 1H), 5.11 (s, 1H), 4.10 (s, 1H), 3.87 (d, J=0.8 Hz, 3H), 3.28 (s, 1H), 2.90 (p, J=7.6 Hz, 1H), 1.60 (s, 3H), 0.75 (d, J=7.2 Hz, 3H) ppm; alcohol OH not observed. ESI-MS m/z calc. 489.13232, 490.3 (M+1)⁺; 488.2 (M−1); Retention time: 0.71 minutes.

### Step 9:

(2-Bromoethoxy) (tert-butyl)dimethylsilane (40 μL, 0.186 mmol) was added to a mixture of methyl 4-((2R,3S,4S,5R)-3-(6-(difluoromethyl)-2-hydroxypyridin-3-yl)-4,5-dimethyl-5-(trifluoromethyl)tetrahydrofuran-2-carboxamido) picolinate (70 mg, 0.143 mmol) and Cs₂CO₃ (134 mg, 0.411 mmol) in MeCN (2 mL). The reaction was heated at 80° C. overnight. The reaction mixture was partitioned between water and EtOAc. The aqueous layer was separated and extracted with EtOAc (5×). The combined organic layers were washed with brine, dried over MgSO₄, filtered, and concentrated in vacuo. Purification by flash chromatography (SiO₂, 0 to 100% EtOAc in heptane) gave methyl 4-((2R,3S,4S,5R)-3-(2-(2-((tert-butyldimethylsilyl)oxy)ethoxy)-6-(difluoromethyl)pyridin-3-yl)-4,5-dimethyl-5-(trifluoromethyl)tetrahydrofuran-2-carboxamido) picolinate (25 mg, 27%). ¹H NMR (500 MHz, Chloroform-d) δ 8.63 (d, J=5.5 Hz, 1H), 8.61 (s, 1H), 8.07 (d, J=2.2 Hz, 1H), 7.91 (dd, J=5.5, 2.2 Hz, 1H), 7.84 (d, J=7.6 Hz, 1H), 7.26 (d, J=7.4 Hz, 1H), 6.49 (t, J=55.6 Hz, 1H), 5.12 (d, J=11.2 Hz, 1H), 4.53-4.33 (m, 2H), 4.05 (dd, J=11.2, 7.7 Hz, 1H), 4.00 (s, 3H), 3.96-3.89 (m, 2H), 3.01 (h, J=7.1, 6.6 Hz, 1H), 1.70 (s, 3H), 0.85 (s, 9H), 0.78-0.73 (m, 3H), 0.03 (s, 6H) ppm. ESI-MS m/z calc. 647.245, found 648.4 (M+1)⁺; 646.5 (M−1); Retention time: 1.22 minutes.

### Step 10 and 11:

A solution of methyl 4-((2R,3S,4S,5R)-3-(2-(2-((tert-butyldimethylsilyl)oxy)ethoxy)-6-(difluoromethyl)pyridin-3-yl)-4,5-dimethyl-5-(trifluoromethyl)tetrahydrofuran-2-carboxamido) picolinate (25 mg, 0.039 mmol) in a methanolic NH₃ solution (2 mL of 7 M, 14.00 mmol) was stirred at ambient temperature overnight. The reaction mixture was concentrated in vacuo. The residue was dissolved in a mixture of DCM (1 mL) and TFA (100 μL, 1.298 mmol). The reaction mixture was stirred at ambient temperature for 4 h. The mixture was concentrated in vacuo and azeotroped with DCM (3×) to remove residual TFA. Purification by reverse phase preparative chromatography gave 4-((2R,3S,4S,5R)-3-(6-(difluoromethyl)-2-(2-hydroxyethoxy)pyridin-3-yl)-4,5-dimethyl-5-(trifluoromethyl)tetrahydrofuran-2-carboxamido) picolinamide (Trifluoroacetate salt) (208, 4 mg, 16% over two steps). ¹H NMR (500 MHZ, DMSO-d₆) δ 10.65 (s, 1H), 8.49 (d, J=5.5 Hz, 1H), 8.31 (d, J=2.2 Hz, 1H), 8.06 (d, J=2.8 Hz, 1H), 7.92-7.81 (m, 2H), 7.60 (d, J=2.8 Hz, 1H), 7.29 (d, J=7.6 Hz, 1H), 6.84 (t, J=55.0 Hz, 1H), 5.22 (d, J=9.6 Hz, 1H), 4.34 (ddt, J=37.7, 10.7, 4.9 Hz, 2H), 4.22 (t, J=8.7 Hz, 1H), 3.74 (t, J=5.0 Hz, 2H), 2.99 (p, J=7.4 Hz, 1H), 1.63 (s, 3H), 0.70 (d, J=7.4 Hz, 3H) ppm; OH alcohol not observed. ESI-MS m/z calc. 518.1589, found 519.3 (M+1)⁺; 517.2 (M−1); Retention time: 2.88 minutes.

### Example 25

### rel-4-((2S,3R,4R,5S)-3-(7-ethoxy-2,2-difluorobenzo[d][1,3]dioxol-4-yl)-4,5-dimethyl-5-(trifluoromethyl)tetrahydrofuran-2-carboxamido) picolinamide (209) and

### rel-4-((2R,3S,4S,5R)-3-(7-ethoxy-2,2-difluorobenzo[d][1,3]dioxol-4-yl)-4,5-dimethyl-5-(trifluoromethyl)tetrahydrofuran-2-carboxamido) picolinamide (210)

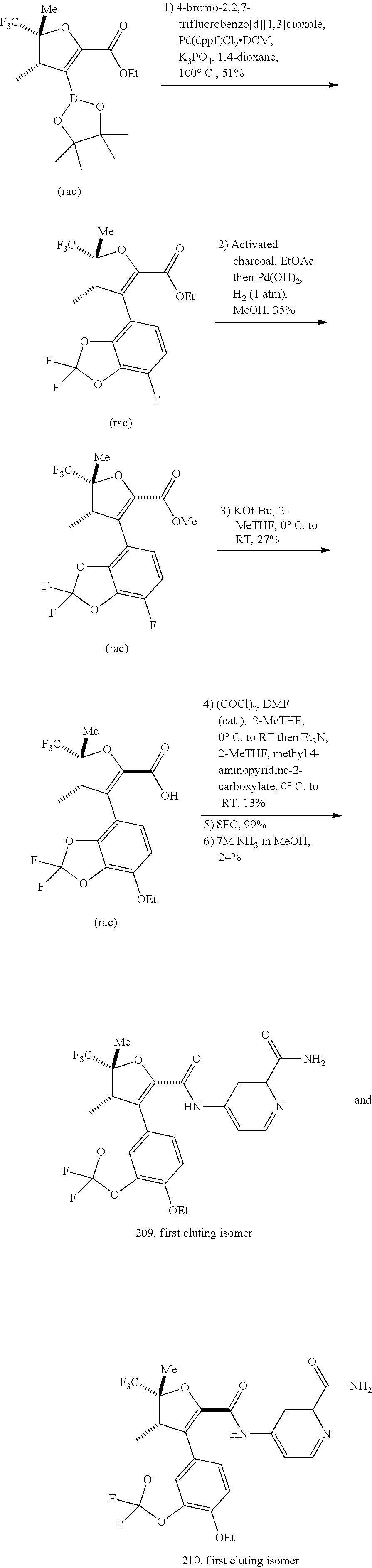

### Step 1:

2 M K₃PO₄ (8.5 mL, 17.00 mmol) was added to a solution of ethyl rac-(4S,5R)-4,5-dimethyl-3-(4,4,5,5-tetramethyl-1,3,2-dioxaborolan-2-yl)-5-(trifluoromethyl)-4,5-dihydrofuran-2-carboxylate (Product of Example 13, Step 2, 3.00 g, 8.238 mmol), 4-bromo-2,2,7-trifluorobenzo[d][1,3]dioxole (Intermediate J, 2 g, 7.843 mmol) and Pd(dppf) Cl₂·CH₂Cl₂ (400 mg, 0.490 mmol) in 1,4-dioxane (60 mL). The reaction mixture was degassed and flushed with nitrogen (×3). The mixture was stirred at 100° C. for 2 h. The reaction mixture was cooled down to ambient temperature and partitioned between water and ethyl acetate. The aqueous layer was separated and extracted with ethyl acetate (×3). The combined organic extracts were passed through a Whatman 1PS hydrophobic phase separator filter paper. The filtrates were concentrated in vacuo to give a brown oil. Purification by flash chromatography (24 g SiO₂, 0 to 70% EtOAc in heptane; then 40 g SiO₂, 0 to 50% EtOAc in heptane) gave ethyl rac-(4S,5R)-4,5-dimethyl-3-(2,2,7-trifluorobenzo[d][1,3]dioxol-4-yl)-5-(trifluoromethyl)-4,5-dihydrofuran-2-carboxylate (2.186 g, 51%) as a pale yellow oil. ¹H NMR (400 MHZ, Chloroform-d) δ 6.91 (dd, J=9.0, 4.7 Hz, 1H), 6.84 (t, J=9.1 Hz, 1H), 4.13 (q, J=7.1 Hz, 2H), 3.49-3.39 (m, 1H), 1.67-1.58 (m, 3H), 1.26 (t, J=7.1 Hz, 3H), 1.12 (t, J=7.1 Hz, 3H) ppm. ESI-MS m/z calc. 412.07455, found 413.1 (M+1)⁺; Retention time: 1.12 minutes.

### Step 2:

Ethyl rac-(4S,5R)-4,5-dimethyl-3-(2,2,7-trifluorobenzo[d][1,3]dioxol-4-yl)-5-(trifluoromethyl)-4,5-dihydrofuran-2-carboxylate (2.186 g, 5.302 mmol) was dissolved in EtOAc and stirred with activated charcoal overnight at ambient temperature. The mixture was filtered through a pad of celite. The liquors were concentrated in vacuo to give a pale yellow oil. The oil was dissolved in methanol (20 mL) and added to a flask flushed with nitrogen and containing palladium hydroxide (1 g of 20% w/w, 1.424 mmol). The reaction mixture was stirred under an atmospheric pressure of hydrogen for 5 days. The mixture was filtered through a celite cartridge, and washed with MeOH and water to quench the catalyst. The filtrates were concentrated in vacuo to give a pale yellow oil as a mixture of product and starting material. The mixture was dissolved in Methanol (20 mL) and added to a flask flushed with nitrogen and containing palladium hydroxide (1 g of 20% w/w, 1.424 mmol). The reaction mixture was stirred under an atmospheric pressure of hydrogen for 48 h. The mixture was filtered through a celite cartridge, and washed with MeOH and water to quench the catalyst. The filtrates were concentrated in vacuo to give a pale yellow oil. Purification by flash chromatography (4 g SiO₂, 0 to 100% EtOAc in heptane) gave methyl rac-(2S,3S,4S,5R)-4,5-dimethyl-3-(2,2,7-trifluorobenzo[d][1,3]dioxol-4-yl)-5-(trifluoromethyl)tetrahydrofuran-2-carboxylate (740.5 mg, 35%) as a white solid and (2S,3S,4S,5R)-4,5-dimethyl-3-(2,2,7-trifluorobenzo[d][1,3]dioxol-4-yl)-5-(trifluoromethyl)tetrahydrofuran-2-carboxylate as the major stereoisomer. ESI-MS m/z calc. 400.07455, Retention time: 1.1 minutes; no mass ionisation.

### Step 3:

Potassium tert-butoxide (650 mg, 5.793 mmol) was added to an ice-cold stirred solution of methyl rac-(2S,3S,4S,5R)-4,5-dimethyl-3-(2,2,7-trifluorobenzo[d][1,3]dioxol-4-yl)-5-(trifluoromethyl)tetrahydrofuran-2-carboxylate (740.5 mg, 1.850 mmol) in 2-MeTHF (10 mL). The reaction mixture was stirred for 1 h at ambient temperature. The mixture was partitioned between ethyl acetate and 1 N NaOH. The organic phase was separated and washed with 1 M NaOH (×2). The combined organic extracts were passed through a Whatman 1PS hydrophobic phase separator filter paper. The filtrates were concentrated in vacuo to give a yellow oil. Purification by flash chromatography (12 g SiO₂, 0 to 100% 3:1 EtOAc: EtOH containing 2% of NH₄OH in water (28-30% NH₃ basis) in heptane) gave rac-(2R,3S,4S,5R)-3-(7-ethoxy-2,2-difluorobenzo[d][1,3]dioxol-4-yl)-4,5-dimethyl-5-(trifluoromethyl)tetrahydrofuran-2-carboxylic acid (517.9 mg, 27%). ESI-MS m/z calc. 412.09454, found 411.0 (M−1); Retention time: 0.66 minutes.

### Step 4:

Oxalyl chloride (70.45 μL, 0.808 mmol) was carefully added to an ice-cold solution of rac-(2R,3S,4S,5R)-3-(7-ethoxy-2,2-difluorobenzo[d][1,3]dioxol-4-yl)-4,5-dimethyl-5-(trifluoromethyl)tetrahydrofuran-2-carboxylic acid (172.6 mg, 0.282 mmol) and DMF (10.07 μL, 0.130 mmol) in 2-MeTHF (5 mL). The reaction mixture was stirred and warmed up to ambient temperature over 30 min. The mixture was concentrated in vacuo. The residue, dissolved in 2-MeTHF (5 mL), was added to an ice-cold and stirred solution of methyl 4-aminopyridine-2-carboxylate (60.40 mg, 0.353 mmol) and Et₃N (201.4 μL, 1.445 mmol) in 2-MeTHF (5 mL). The reaction mixture was stirred and warmed to ambient temperature over 18 h. The mixture was quenched with water (5 mL) and the layers were separated. The aqueous layer was extracted with EtOAc (2×10 mL). The combined organic extracts were passed through a Whatman 1PS hydrophobic phase separator filter paper. The filtrates were concentrated in vacuo to give an oil (182.8 mg). Purification by flash chromatography (12 g SiO₂, 0 to 50% EtOAc in heptane) gave methyl rac-4-((2R,3S,4S,5R)-3-(7-ethoxy-2,2-difluorobenzo[d][1,3]dioxol-4-yl)-4,5-dimethyl-5-(trifluoromethyl)tetrahydrofuran-2-carboxamido) picolinate (28.6 mg, 13%). ¹H NMR (400 MHz, Chloroform-d) δ 8.77-8.66 (m, 2H), 8.17 (s, 1H), 7.99 (d, J=5.6 Hz, 1H), 7.03 (d, J=8.9 Hz, 1H), 6.75 (d, J=8.9 Hz, 1H), 5.16 (d, J=10.6 Hz, 1H), 4.22 (q, J=7.0 Hz, 2H), 4.04 (d, J=6.6 Hz, 3H), 3.99 (dd, J=10.7, 8.0 Hz, 1H), 2.78 (p, J=7.6 Hz, 1H), 1.91-1.61 (m, 3H), 1.48 (t, J=7.0 Hz, 3H), 0.99-0.86 (m, 3H) ppm. ESI-MS m/z calc. 546.1425, found 547.2 (M+1)⁺; 545.2 (M−1); Retention time: 1.06 minutes.

### Step 5:

The enantiomers of methyl rac-4-((2R,3S,4S,5R)-3-(7-ethoxy-2,2-difluorobenzo[d][1,3]dioxol-4-yl)-4,5-dimethyl-5-(trifluoromethyl)tetrahydrofuran-2-carboxamido) picolinate (28.6 mg, 0.037 mmol) were separated by chiral SFC using a Lux Cellulose-2 column, 5 μm particle size, 25 cm×10 mm from Phenomenex, Inc. (Mobile phase: 50% methanol (supplemented with 20 mM NH₃), 50% CO₂; Flow rate: 75 mL/min, System pressure: 100 bar) on a Minigram SFC instrument from Berger Instruments to give:

First Eluting Isomer (rt=2.38 min): methyl rel-4-((2S,3R,4R,5S)-3-(7-ethoxy-2,2-difluorobenzo[d][1,3]dioxol-4-yl)-4,5-dimethyl-5-(trifluoromethyl)tetrahydrofuran-2-carboxamido) picolinate (12.4 mg, 98%). ESI-MS m/z calc. 546.1425, found 547.1 (M+1)⁺; 545.2 (M−1); Retention time: 3.68 minutes.

Second Eluting Isomer (rt=3.15 min): methyl rel-4-((2R,3S,4S,5R)-3-(7-ethoxy-2,2-difluorobenzo[d][1,3]dioxol-4-yl)-4,5-dimethyl-5-(trifluoromethyl)tetrahydrofuran-2-carboxamido) picolinate (11.5 mg, 100%). ESI-MS m/z calc. 546.1425, found 547.2 (M+1)⁺; 545.2 (M−1); Retention time: 3.68 minutes.

### Step 6:

Methanolic ammonia (200 μL of 7 M, 1.400 mmol) was added to a solution of methyl rel-4-((2S,3R,4R,5S)-3-(7-ethoxy-2,2-difluorobenzo[d][1,3]dioxol-4-yl)-4,5-dimethyl-5-(trifluoromethyl)tetrahydrofuran-2-carboxamido) picolinate (12.4 mg, 0.023 mmol) in methanol (1 mL). The reaction mixture was sealed and stirred at ambient temperature for 20 h. The reaction mixture was concentrated in vacuo. Purification by reverse phase HPLC-MS using a X-bridge C18 OBD column (150×19 mm, 5 mm particle size) from Waters (Mobile phase: acetonitrile in water (supplemented with 0.1% ammonium hydroxide); Flow rate: 19 mL/min; Column temperature: 25° C.) gave rel-4-((2S,3R,4R,5S)-3-(7-ethoxy-2,2-difluorobenzo[d][1,3]dioxol-4-yl)-4,5-dimethyl-5-(trifluoromethyl)tetrahydrofuran-2-carboxamido) picolinamide (209, 2.9 mg, 24%) as a white solid. ¹H NMR (400 MHZ, Chloroform-d) δ 8.61 (s, 1H), 8.47 (d, J=5.5 Hz, 1H), 8.15 (dd, J=5.5, 2.3 Hz, 1H), 7.92 (d, J=2.2 Hz, 1H), 7.84 (s, 1H), 7.01 (d, J=8.8 Hz, 1H), 6.72 (d, J=8.9 Hz, 1H), 5.54 (s, 1H), 5.11 (d, J=10.6 Hz, 1H), 4.20 (q, J=7.0 Hz, 2H), 3.94 (dd, J=10.7, 8.0 Hz, 1H), 2.74 (p, J=7.6 Hz, 1H), 1.69 (d, J=1.2 Hz, 3H), 1.45 (t, J=7.0 Hz, 3H), 0.90 (dd, J=7.6, 2.3 Hz, 3H) ppm. ¹⁹F NMR (376 MHz, Chloroform-d) δ −49.35 (d, J=5.1 Hz), −74.60 ppm. ESI-MS m/z calc. 531.1429, found 532.1 (M+1)⁺; 530.1 (M−1); Retention time: 3.58 minutes.

Methyl rel-4-((2R,3S,4S,5R)-3-(7-ethoxy-2,2-difluorobenzo[d][1,3]dioxol-4-yl)-4,5-dimethyl-5-(trifluoromethyl)tetrahydrofuran-2-carboxamido) picolinate (210, 11.5 mg, 0.021 mmol) (Second Eluting Isomer from SFC separation) was treated in the same way.

### Example 26

### 4-((2R,3S,4S,5R)-3-(3,4-difluoro-2-((oxetan-3-yloxy)methyl)phenyl)-4,5-dimethyl-5-(trifluoromethyl)tetrahydrofuran-2-carboxamido) picolinamide (211)

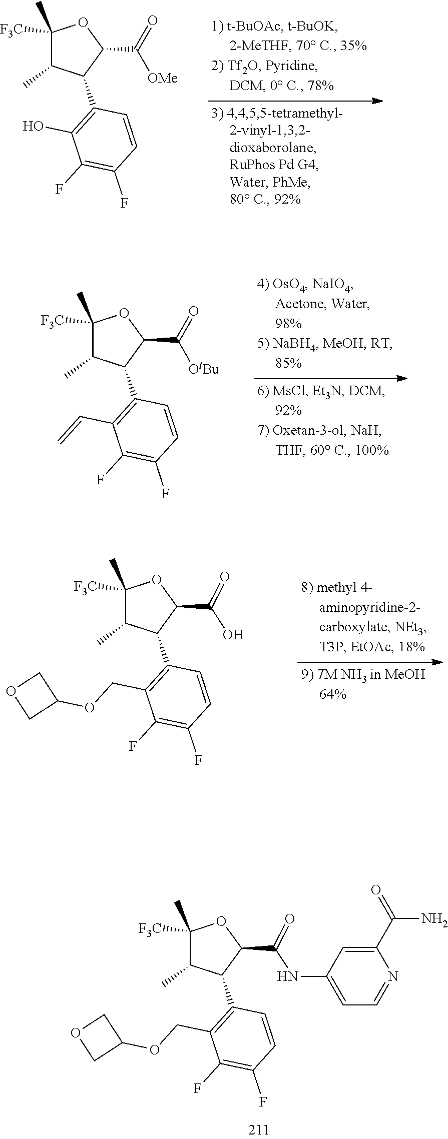 [SMILES: COC(=O)[C@H]1O[C@@](C)(C(F)(F)F)[C@@H](C)[C@H]1c1ccc(F)c(F)c1O ; C=Cc1c([C@H]2[C@H](C(=O)OC(C)(C)C)O[C@@](C)(C(F)(F)F)[C@H]2C)ccc(F)c1F ; C[C@H]1[C@@H](c2ccc(F)c(F)c2COC2COC2)[C@H](C(=O)O)O[C@@]1(C)C(F)(F)F ; C[C@H]1[C@@H](c2ccc(F)c(F)c2COC2COC2)[C@H](C(=O)Nc2ccnc(C(N)=O)c2)O[C@@]1(C)C(F)(F)F]

### Step 1:

Potassium tert-butoxide (4.6 g, 40.99 mmol) was added in one portion to a solution of methyl(2S,3S,4S,5R)-3-(3,4-difluoro-2-hydroxyphenyl)-4,5-dimethyl-5-(trifluoromethyl)tetrahydrofuran-2-carboxylate (Product of Example 2, Step 2, 10 g, 28.23 mmol) and t-BuOAc (10 mL, 119.0 mmol) in 2-MeTHF (100 mL). The reaction mixture was heated to 70° C. and stirred for 1 h. The mixture was concentrated in vacuo to give a yellow solid. Purification by flash chromatography (240 g SiO₂, 0 to 20% EtOAc in heptane) gave tert-butyl(2R,3S,4S,5R)-3-(3,4-difluoro-2-hydroxyphenyl)-4,5-dimethyl-5-(trifluoromethyl)tetrahydrofuran-2-carboxylate (4.16 g, 35%) as a white solid. ¹H NMR (400 MHZ, DMSO-d₆) δ 10.40 (s, 1H), 7.05 (ddd, J=8.5, 5.9, 2.1 Hz, 1H), 6.84 (ddd, J=10.2, 8.8, 7.5 Hz, 1H), 4.91 (d, J=10.6 Hz, 1H), 4.09-3.98 (m, 1H), 2.71 (p, J=7.5 Hz, 1H), 1.54-1.41 (m, 3H), 1.27 (s, 9H), 0.70 (td, J=4.5, 2.3 Hz, 3H) ppm. ESI-MS m/z calc. 396.136, found 395.4 (M−1); Retention time: 1.04 minutes.

### Step 2:

Tf₂O (5 mL of 1 M in DCM, 5.0 mmol) was added dropwise to a solution of tert-butyl(2R,3S,4S,5R)-3-(3,4-difluoro-2-hydroxyphenyl)-4,5-dimethyl-5-(trifluoromethyl)tetrahydrofuran-2-carboxylate (1.78 g, 4.132 mmol) and pyridine (700 μL, 8.655 mmol) in DCM (15 mL). The reaction mixture was stirred at 0° C. for 2 h. The mixture was diluted with DCM (100 mL). The organic phase was separated, washed with water (80 mL) and brine (50 mL), dried over MgSO₄, filtered, and concentrated in vacuo. Purification by flash chromatography (SiO₂, 0 to 100% EtOAc in heptane) gave tert-butyl(2R,3S,4S,5R)-3-(3,4-difluoro-2-(((trifluoromethyl) sulfonyl)oxy)phenyl)-4,5-dimethyl-5-(trifluoromethyl)tetrahydrofuran-2-carboxylate (1.702 g, 78%) as a pale yellow crystalline solid. ¹H NMR (500 MHZ, DMSO-d₆) δ 7.72 (td, J=9.4, 7.7 Hz, 1H), 7.66-7.59 (m, 1H), 5.15 (d, J=10.3 Hz, 1H), 3.96 (dd, J=10.3, 7.4 Hz, 1H), 2.68 (p, J=7.4 Hz, 1H), 1.51 (s, 3H), 1.26 (s, 9H), 0.77 (dd, J=7.5, 2.4 Hz, 3H) ppm. ESI-MS m/z calc. 528.08527, Retention time: 1.22 minutes; no mass ionisation.

### Step 3:

A mixture of tert-butyl(2R,3S,4S,5R)-3-(3,4-difluoro-2-(((trifluoromethyl) sulfonyl)oxy)phenyl)-4,5-dimethyl-5-(trifluoromethyl)tetrahydrofuran-2-carboxylate (3.5 g, 6.094 mmol), 4,4,5,5-tetramethyl-2-vinyl-1,3,2-dioxaborolane (3.6 mL, 21.22 mmol), Cs₂CO₃ (4.3 g, 13.20 mmol) and RuPhos Pd G4 (750 mg, 0.882 mmol) was suspended in toluene (60 mL) and water (5 mL). The resultant suspension was heated to 80° C. for 5 h. The mixture was cooled to ambient temperature and partitioned between MTBE (100 mL) and water (100 mL). The aqueous layer was extracted with MTBE (2×50 mL). The combined organic extracts were washed with brine (100 mL), dried over MgSO₄, filtered, and concentrated in vacuo to give a dark brown oil. Purification by flash chromatography (12 g SiO₂, 0 to 40% EtOAc in heptane then, 12 g SiO₂, 0 to 100% EtOAc in heptane) gave tert-butyl(2R,3S,4S,5R)-3-(3,4-difluoro-2-vinylphenyl)-4,5-dimethyl-5-(trifluoromethyl)tetrahydrofuran-2-carboxylate (2.42 g, 92%). ¹H NMR (400 MHZ, DMSO-d₆) δ 7.35 (dt, J=10.1, 8.5 Hz, 1H), 7.26 (ddd, J=9.0, 5.0, 1.6 Hz, 1H), 6.78 (dd, J=17.7, 11.6 Hz, 1H), 5.76 (ddd, J=11.5, 1.6, 0.6 Hz, 1H), 5.65 (dt, J=17.7, 1.7 Hz, 1H), 4.97 (d, J=10.7 Hz, 1H), 3.97 (td, J=11.0, 7.5 Hz, 1H), 2.67 (h, J=7.8 Hz, 1H), 1.56-1.46 (m, 3H), 1.24 (s, 9H), 0.70 (dq, J=7.5, 2.3 Hz, 3H) ppm. ESI-MS m/z calc. 406.15674, Retention time: 1.19 minutes; no mass ionisation.

### Step 4:

NaIO₄ (350 mg, 1.636 mmol) and OsO₄ (400 mg of 0.3 mmol/g, 0.120 mmol) were successively added to a solution of tert-butyl(2R,3S,4S,5R)-3-(3,4-difluoro-2-vinylphenyl)-4,5-dimethyl-5-(trifluoromethyl)tetrahydrofuran-2-carboxylate (140 mg, 0.270 mmol) in acetone (2 mL) and water (500 μL) under a nitrogen atmosphere. The reaction mixture was stirred at ambient temperature for 20 h. The mixture was partitioned between MTBE (25 mL) and water (25 mL). The aqueous phase was separated and extracted with MTBE (2×25 mL). The combined organic extracts were dried over MgSO₄, filtered, and concentrated in vacuo to give tert-butyl(2R,3S,4S,5R)-3-(3,4-difluoro-2-formylphenyl)-4,5-dimethyl-5-(trifluoromethyl)tetrahydrofuran-2-carboxylate (111 mg, 98%) as a yellow oil which solidifies to a pale yellow solid on standing, and which was used in the next step without further purification. 1H NMR (400 MHZ, DMSO-d₆) δ 10.41 (d, J=0.7 Hz, 1H), 7.78 (dt, J=10.1, 8.6 Hz, 1H), 7.49 (dd, J=9.0, 4.5 Hz, 1H), 5.09 (d, J=10.8 Hz, 1H), 4.66 (dd, J=10.8, 7.2 Hz, 1H), 2.77-2.65 (m, 1H), 1.58-1.49 (m, 3H), 1.24 (s, 9H), 0.69 (dd, J=7.4, 2.3 Hz, 3H) ppm. ESI-MS m/z calc. 408.136, Retention time: 1.11 minutes; no mass ionisation.

### Step 5:

NaBH₄ (35 mg, 0.925 mmol) was added to a solution of tert-butyl(2R,3S,4S,5R)-3-(3,4-difluoro-2-formylphenyl)-4,5-dimethyl-5-(trifluoromethyl)tetrahydrofuran-2-carboxylate (200 mg, 0.490 mmol) in MeOH (5 mL) at ambient temperature. The reaction mixture was stirred at ambient temperature for 2 h. The mixture was partitioned between a saturated aqueous NH₄Cl solution (20 mL) and DCM (30 mL). The aqueous phase was separated and extracted with DCM (2×30 mL). The combined organic extracts were dried over MgSO₄, filtered, and concentrated in vacuo. Purification by flash chromatography (40 g SiO₂, 0 to 100% EtOAc in heptane) gave tert-butyl(2R,3S,4S,5R)-3-(3,4-difluoro-2-(hydroxymethyl)phenyl)-4,5-dimethyl-5-(trifluoromethyl)tetrahydrofuran-2-carboxylate (183 mg, 85%) as a colourless oil. ¹H NMR (500 MHZ, DMSO-d₆) δ 7.36 (dt, J=10.1, 8.5 Hz, 1H), 7.28 (ddd, J=9.1, 5.0, 1.4 Hz, 1H), 5.35 (t, J=5.2 Hz, 1H), 4.96 (d, J=10.9 Hz, 1H), 4.61 (ddd, J=12.4, 5.0, 2.5 Hz, 1H), 4.52 (ddd, J=12.3, 5.6, 2.7 Hz, 1H), 4.23 (dd, J=11.0, 7.4 Hz, 1H), 2.77 (h, J=7.3 Hz, 1H), 1.53 (s, 3H), 1.22 (s, 9H), 0.72 (dt, J=7.5, 2.5 Hz, 3H) ppm. ESI-MS m/z calc. 410.15164, Retention time: 1.02 minutes; no mass ionisation.

### Step 6:

Methanesulfonyl chloride (40 μL, 0.517 mmol) was added dropwise to a solution of tert-butyl(2R,3S,4S,5R)-3-(3,4-difluoro-2-(hydroxymethyl)phenyl)-4,5-dimethyl-5-(trifluoromethyl)tetrahydrofuran-2-carboxylate (60 mg, 0.146 mmol) and Et₃N (50 μL, 0.3587 mmol) in DCM (1.5 mL) at 0° C. The reaction mixture was warmed to ambient temperature and stirred for 2 h. The mixture was diluted with DCM (15 mL) and washed with a saturated NaHCO₃ solution (15 mL). The aqueous phase was separated and extracted with DCM (3×10 mL). The combined organic extracts were dried over MgSO₄, filtered and concentrated in vacuo to give tert-butyl(2R,3S,4S,5R)-3-(3,4-difluoro-2-(((methylsulfonyl)oxy)methyl)phenyl)-4,5-dimethyl-5-(trifluoromethyl)tetrahydrofuran-2-carboxylate (90 mg, 92%) as a colourless oil. ¹H NMR (500 MHZ, DMSO-d₆) δ 7.50 (dt, J=10.0, 8.6 Hz, 1H), 7.41-7.34 (m, 1H), 5.06 (d, J=10.5 Hz, 1H), 4.96 (dd, J=12.3, 1.7 Hz, 1H), 4.85 (dd, J=12.2, 2.4 Hz, 1H), 4.16-4.07 (m, 1H), 2.89-2.80 (m, 1H), 2.35 (s, 3H), 1.58 (d, J=1.1 Hz, 3H), 1.24 (s, 9H), 0.73-0.69 (m, 3H) ppm. ESI-MS m/z calc. 488.1292, Retention time: 1.16 minutes; no mass ionisation.

### Step 7:

A stirred suspension of oxetan-3-ol (40 μL, 0.630 mmol) and NaH (25 mg, 0.625 mmol) in THF (2 mL) was stirred at ambient temperature for 15 min. tert-Butyl(2R,3S,4S,5R)-3-(3,4-difluoro-2-(((methylsulfonyl)oxy)methyl)phenyl)-4,5-dimethyl-5-(trifluoromethyl)tetrahydrofuran-2-carboxylate (90 mg, 0.134 mmol) was added to the suspension. The mixture was heated to 60° C. for 16 h. The reaction mixture was concentrated in vacuo and diluted with a saturated NH₄Cl solution (20 mL). The mixture was acidified to pH 4 with 1M HCl. This aqueous phase was extracted with EtOAc (3×15 mL). The combined organic extracts were dried over MgSO₄, filtered, and concentrated in vacuo to give (2R,3S,4S,5R)-3-(3,4-difluoro-2-((oxetan-3-yloxy)methyl)phenyl)-4,5-dimethyl-5-(trifluoromethyl)tetrahydrofuran-2-carboxylic acid (71 mg, 100%) as a dark brown oil, which was used in the next step without further purification. ESI-MS m/z calc. 410.11526, found 411.5 (M+1)⁺; 409.4 (M−1)⁻; Retention time: 0.56 minutes.

### Step 8:

T3P in EtOAc (200 μL, 0.673 mmol) was added to a stirred solution of (2R,3S,4S,5R)-3-(3,4-difluoro-2-((oxetan-3-yloxy)methyl)phenyl)-4,5-dimethyl-5-(trifluoromethyl)tetrahydrofuran-2-carboxylic acid (95 mg, 0.171 mmol), methyl 4-aminopyridine-2-carboxylate (35 mg, 0.230 mmol) and Et₃N (100 μL, 0.718 mmol) in EtOAc (4 mL). The reaction mixture was stirred at ambient temperature for 1 h. The mixture was diluted with EtOAc (15 mL) and washed with water (20 mL). The aqueous phase was separated and extracted with EtOAc (3×10 mL). The combined organic extracts were dried over MgSO₄, filtered and concentrated in vacuo. Purification by flash chromatography (4 g SiO₂, 0 to 100% EtOAc in heptane) gave methyl 4-((2R,3S,4S,5R)-3-(3,4-difluoro-2-((oxetan-3-yloxy)methyl)phenyl)-4,5-dimethyl-5-(trifluoromethyl)tetrahydrofuran-2-carboxamido) picolinate (20 mg, 18%) as a colourless oil. ¹H NMR (500 MHZ, DMSO-d₆) δ 10.65 (s, 1H), 8.57 (d, J=5.5 Hz, 1H), 8.36 (d, J=2.1 Hz, 1H), 7.85 (dd, J=5.5, 2.2 Hz, 1H), 7.48 (q, J=9.1 Hz, 1H), 7.31 (dd, J=9.2, 4.4 Hz, 1H), 5.17 (d, J=10.4 Hz, 1H), 4.68-4.60 (m, 3H), 4.60-4.52 (m, 2H), 4.42-4.33 (m, 3H), 3.87 (s, 3H), 2.82 (t, J=7.6 Hz, 1H), 1.65 (s, 3H), 0.74 (d, J=7.4 Hz, 3H) ppm. ESI-MS m/z calc. 544.16327, found 545.6 (M+1)⁺; 543.5 (M−1); Retention time: 0.9 minutes.

### Step 9:

Ammonia in MeOH (1 mL of 7 M, 7.0 mmol) was added to methyl 4-((2R,3S,4S,5R)-3-(3,4-difluoro-2-((oxetan-3-yloxy)methyl)phenyl)-4,5-dimethyl-5-(trifluoromethyl)tetrahydrofuran-2-carboxamido) picolinate (20 mg, 0.03490 mmol) under a nitrogen atmosphere. The reaction was stirred at ambient temperature for 20 h in a sealed vial. The mixture was concentrated in vacuo. The residue was triturated with heptane and azeotroped with DCM to give 4-((2R,3S,4S,5R)-3-(3,4-difluoro-2-((oxetan-3-yloxy)methyl)phenyl)-4,5-dimethyl-5-(trifluoromethyl)tetrahydrofuran-2-carboxamido) picolinamide (211, 12.2 mg, 64%) as a white solid. ¹H NMR (500 MHZ, DMSO-d₆) δ 10.61 (s, 1H), 8.49 (d, J=5.5 Hz, 1H), 8.27 (d, J=2.2 Hz, 1H), 8.06 (s, 1H), 7.83 (dd, J=5.5, 2.2 Hz, 1H), 7.62 (s, 1H), 7.48 (q, J=9.0 Hz, 1H), 7.31 (dd, J=8.8, 4.5 Hz, 1H), 5.16 (d, J=10.4 Hz, 1H), 4.74-4.48 (m, 5H), 4.45-4.28 (m, 3H), 2.88-2.76 (m, 1H), 1.65 (s, 3H), 0.75 (d, J=7.4 Hz, 3H) ppm. ESI-MS m/z calc. 529.16364, found 530.6 (M+1)⁺; 528.5 (M−1); Retention time: 3.0 minutes.

### Example 27

### 4-((2R,3S,4S,5R)-3-(2-((1s,3R)-3-hydroxycyclobutoxy)-3-(trifluoromethyl)phenyl)-4,5-dimethyl-5-(trifluoromethyl)tetrahydrofuran-2-carboxamido) picolinamide (212)

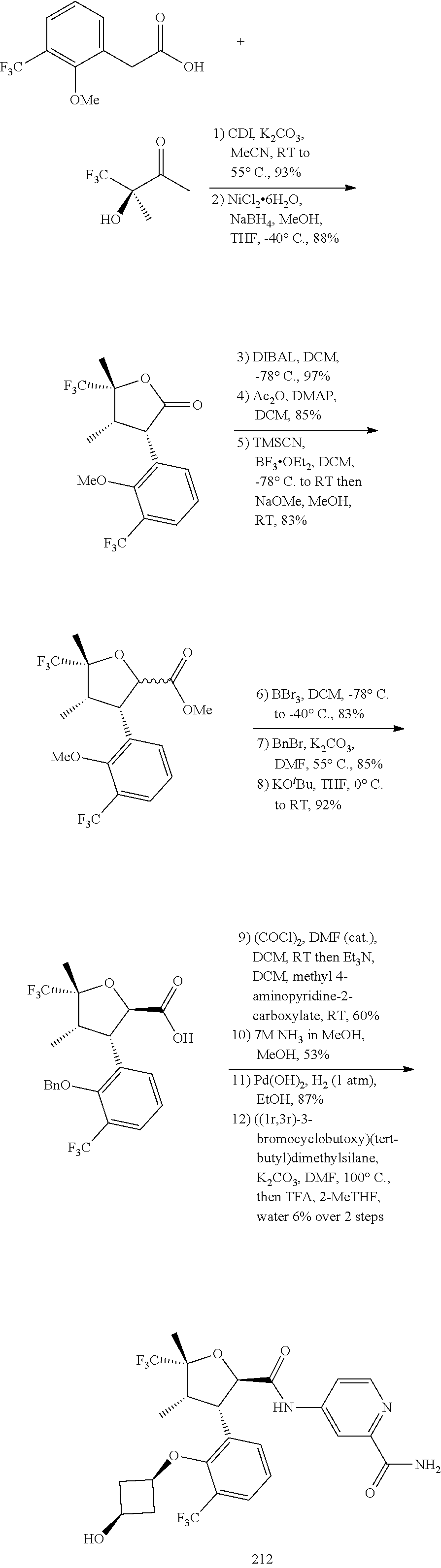

### Step 1:

In a flame-dried flask, under an atmosphere of argon, CDI (152 mg, 0.937 mmol) was dissolved in MeCN (5 mL) and the mixture was heated and stirred at 50° C. for 30 min. 2-(2-Methoxy-3-(trifluoromethyl)phenyl) acetic acid (Intermediate E, 200 mg, 0.854 mmol) was added and the mixture was heated at 50° C. for 1 h. (R)-4,4,4-trifluoro-3-hydroxy-3-methylbutan-2-one (Intermediate C, 133 mg, 0.852 mmol) and K₂CO₃ (154 mg, 1.114 mmol) were successively added to the reaction mixture under heating condition. The reaction was stirred at 50° C. for 16 h. The mixture was cooled to ambient temperature and partitioned between water and EtOAc (50 mL). The organic layer was separated, washed with 2 N HCl (3 mL), dried over Na₂SO₄, filtered, and concentrated in vacuo to give (R)-3-(2-methoxy-3-(trifluoromethyl)phenyl)-4,5-dimethyl-5-(trifluoromethyl) furan-2 (5H)-one (280 mg, 93%) as a yellow solid. ¹H NMR (400 MHz, DMSO-d₆) δ 7.81 (d, J=7.44 Hz, 1H), 7.66 (d, J=7.08 Hz, 1H), 7.47-7.43 (m, 1H), 3.55 (s, 3H), 2.06 (s, 3H), 1.82 (s, 3H) ppm.

### Step 2:

Nickel dichloride hexahydrate (1.710 g, 7.194 mmol) was added to a stirred and previously degassed solution of (R)-3-(2-methoxy-3-(trifluoromethyl)phenyl)-4,5-dimethyl-5-(trifluoromethyl) furan-2 (5H)-one (2.501 g, 7.060 mmol) in MeOH (250 mL) and THF (50 mL) at −40° C. NaBH₄ (1.405 g, 37.14 mmol) was added portionwise and the reaction mixture was stirred at −40° C. for 10 min. Further amounts of NiCl₂ (4×1 eq) and NaBH₄ (4×5 eq) were added portionwise. Upon completion of the reaction, the mixture was quenched by addition of a NH₄Cl solution (100 mL) and was diluted with DCM (100 mL). The reaction mixture was warmed up to ambient temperature and stirred under nitrogen for 30 min. The aqueous phase was separated and extracted with DCM (3×50 mL). The combined organic extracts were dried over MgSO₄, filtered, and concentrated in vacuo to give (3S,4S,5R)-3-(2-methoxy-3-(trifluoromethyl)phenyl)-4,5-dimethyl-5-(trifluoromethyl)dihydrofuran-2 (3H)-one (2.204 g, 88%) as an orange gum. ¹H NMR (400 MHZ, Chloroform-d) δ 7.61 (dd, J=7.9, 1.7 Hz, 1H), 7.49 (dd, J=7.9, 1.6 Hz, 1H), 7.28 (dq, J=7.8, 0.9 Hz, 1H), 4.62 (d, J=9.3 Hz, 1H), 3.88 (s, 3H), 3.00 (dq, J=9.3, 7.5 Hz, 1H), 1.75 (q, J=1.2 Hz, 3H), 0.80 (dq, J=7.5, 2.4 Hz, 3H) ppm. ESI-MS m/z calc. 356.08472, found 355.3 (M−1); Retention time: 1.04 minutes.

### Step 3:

DIBAL (4.4 mL of a 1 M solution in DCM, 4.400 mmol) was added dropwise over 5 min to a stirred solution of (3S,4S,5R)-3-(2-methoxy-3-(trifluoromethyl)phenyl)-4,5-dimethyl-5-(trifluoromethyl)dihydrofuran-2 (3H)-one (1.04 g, 2.511 mmol) in DCM (75 mL) at −78° C. under a nitrogen atmosphere. The reaction mixture was stirred for 20 min. A further portion of DIBAL (2.5 mL of a 1 M solution in DCM, 2.500 mmol) was added dropwise over 5 min and the reaction mixture was stirred at −78° C. under a nitrogen atmosphere for another 15 min. The reaction mixture was quenched by addition of a saturated aqueous NH₄Cl solution (20 mL). The mixture was diluted with DCM (10 mL), warmed to ambient temperature, and stirred a nitrogen atmosphere for an additional 30 min. The mixture was diluted with 1M HCl (˜10 mL) until obtention of phase separation. The aqueous phase was separated and extracted with DCM (2×50 mL). The combined organic phases were dried over MgSO₄, filtered and concentrated in vacuo to give (3S,4S,5R)-3-(2-methoxy-3-(trifluoromethyl)phenyl)-4,5-dimethyl-5-(trifluoromethyl)tetrahydrofuran-2-ol (912 mg, 97%) as a clear, colourless gum. ¹H NMR (500 MHZ, Chloroform-d) δ 7.54 (dd, J=7.8, 1.6 Hz, 1H), 7.50-7.45 (m, 1H), 7.21 (td, J=7.9, 0.9 Hz, 1H), 5.85 (d, J=4.6 Hz, 1H), 3.93 (dd, J=8.7, 4.7 Hz, 1H), 3.86 (s, 3H), 2.95 (p, J=7.8 Hz, 1H), 1.67 (q, J=1.2 Hz, 3H), 0.80 (dq, J=7.7, 2.2 Hz, 3H) ppm; alcohol OH not observed. ESI-MS m/z calc. 358.10037, found 357.4 (M−1); Retention time: 0.98 minutes.

### Step 4:

Ac2O (1.15 mL, 12.19 mmol) was added dropwise to a stirred solution of (3S,4S,5R)-3-(2-methoxy-3-(trifluoromethyl)phenyl)-4,5-dimethyl-5-(trifluoromethyl)tetrahydrofuran-2-ol (1.964 g, 4.057 mmol) and DMAP (542 mg, 4.437 mmol) in DCM (40 mL) at ambient temperature and under a nitrogen atmosphere. The reaction mixture was stirred for 30 min. The mixture was quenched by addition of a saturated aqueous sodium bicarbonate solution (6 mL) and water (4 mL). The aqueous phase was separated and extracted with DCM (2×15 mL). The combined organic phases were washed with a saturated aqueous NH₄Cl solution (20 mL) and brine (20 mL), dried over MgSO₄, filtered, and concentrated in vacuo to give (3S,4S,5R)-3-(2-methoxy-3-(trifluoromethyl)phenyl)-4,5-dimethyl-5-(trifluoromethyl)tetrahydrofuran-2-yl acetate (1.907 g, 85%) as an orange oil. ¹H NMR (500 MHZ, Chloroform-d) δ 7.55 (dd, J=7.8, 1.6 Hz, 1H), 7.49 (d, J=7.9 Hz, 1H), 7.22 (td, J=7.8, 0.9 Hz, 1H), 6.60 (d, J=2.8 Hz, 1H), 4.07 (dd, J=8.8, 2.9 Hz, 1H), 3.83 (s, 3H), 2.96 (p, J=7.8 Hz, 1H), 2.11 (s, 3H), 1.65 (q, J=1.2 Hz, 3H), 0.83 (dq, J=7.6, 2.1 Hz, 3H) ppm. ESI-MS m/z calc. 400.11093, Retention time: 1.08 minutes; no mass ionisation.

### Step 5:

TMSCN (1.1 mL, 8.249 mmol) and BF₃· (OEt)₂ (3 mL, 24.31 mmol) were successively added dropwise over 5 min to a stirred solution of (3S,4S,5R)-3-(2-methoxy-3-(trifluoromethyl)phenyl)-4,5-dimethyl-5-(trifluoromethyl)tetrahydrofuran-2-yl acetate (1.963 g, 3.531 mmol) in DCM (30 mL) at −78° C. and under a nitrogen atmosphere. The reaction mixture was stirred at −78° C. for 30 min. The mixture was warmed to ambient temperature and stirred for an additional 20 min. The mixture was quenched by addition of a saturated aqueous sodium bicarbonate solution (60 mL). The aqueous phase was separated and extracted with DCM (3×20 mL). The combined organic extracts were dried over MgSO₄, filtered, and concentrated in vacuo to give (3S,4S,5R)-3-(2-methoxy-3-(trifluoromethyl)phenyl)-4,5-dimethyl-5-(trifluoromethyl)tetrahydrofuran-2-carbonitrile (1.620 g) as an orange solid. ¹H NMR (500 MHZ, Chloroform-d) δ 7.62 (dd, J=7.7, 1.8 Hz, 1H), 7.32 (dd, J=7.9, 1.8 Hz, 1H), 7.28 (dd, J=7.8, 0.8 Hz, 1H), 5.04 (d, J=9.5 Hz, 1H), 4.32 (dd, J=9.5, 8.3 Hz, 1H), 3.92 (s, 3H), 2.92 (p, J=7.7 Hz, 1H), 1.66 (q, J=1.2 Hz, 3H), 0.78 (dt, J=7.5, 2.3 Hz, 3H) ppm.

NaOMe (2 mL of 25% w/w, 8.746 mmol) was added dropwise over 2 min to a stirred solution of (3S,4S,5R)-3-(2-methoxy-3-(trifluoromethyl)phenyl)-4,5-dimethyl-5-(trifluoromethyl)tetrahydrofuran-2-carbonitrile (1.620 g) in MeOH (15 mL) at ambient temperature and under a nitrogen atmosphere. The reaction mixture was stirred for 75 min. The mixture was quenched by addition of a saturated aqueous citric acid solution (20 mL) and stirred for 15 min. The aqueous phase was separated and extracted with DCM (3×20 mL). The combined organic extracts were washed with brine (30 mL), dried over MgSO₄, filtered, and concentrated in vacuo to give methyl(2R,3S,4S,5R)-3-(2-methoxy-3-(trifluoromethyl)phenyl)-4,5-dimethyl-5-(trifluoromethyl)tetrahydrofuran-2-carboxylate (1.474 g, 83%) as an orange oil. ¹H NMR (500 MHZ, Chloroform-d) δ 7.56 (dd, J=7.9, 1.6 Hz, 1H), 7.49-7.45 (m, 1H), 7.23 (td, J=7.9, 1.0 Hz, 1H), 4.95 (d, J=10.3 Hz, 1H), 4.25 (dd, J=10.3, 8.2 Hz, 1H), 3.86 (s, 3H), 3.72 (s, 3H), 2.81 (p, J=7.7 Hz, 1H), 1.64 (q, J=1.1 Hz, 3H), 0.76 (dq, J=7.4, 2.3 Hz, 3H) ppm. ESI-MS m/z calc. 400.11093, Retention time: 1.07 minutes; no mass ionisation.

### Step 6:

BBr₃ (6 mL of a 1 M solution in heptane, 6.0 mmol) was added dropwise over 10 min to a stirred solution of methyl(2R,3S,4S,5R)-3-(2-methoxy-3-(trifluoromethyl)phenyl)-4,5-dimethyl-5-(trifluoromethyl)tetrahydrofuran-2-carboxylate (1.470 g, 2.938 mmol) in DCM (30 mL) at −78° C. and under a nitrogen atmosphere. The reaction mixture was stirred for 30 min at −78° C., and then for 15 min at −40° C. Another portion of BBr₃ (3 mL of a 1 M solution in heptane, 3.0 mmol) was added dropwise over 5 min to the reaction mixture, which was stirred at −40° C. for an additional 40 min. A final portion of BBr₃ (1.5 mL of a 1 M solution in heptane, 1.5 mmol) was added dropwise over 5 min and the reaction mixture was stirred at −40° C. for another 15 min. The mixture was quenched by addition of a saturated aqueous sodium bicarbonate solution (30 mL). The aqueous phase was separated and extracted with DCM (3×20 mL). The combined organic extracts were dried over MgSO₄, filtered and concentrated in vacuo to give an orange oil. Purification by flash chromatography (SiO₂, 0 to 80% EtOAc in heptane) gave methyl(2R,3S,4S,5R)-3-(2-hydroxy-3-(trifluoromethyl)phenyl)-4,5-dimethyl-5-(trifluoromethyl)tetrahydrofuran-2-carboxylate (1.148 g, 83%) as an orange oil. ¹H NMR (500 MHZ, Chloroform-d) δ 7.43 (d, J=7.9 Hz, 2H), 7.04 (td, J=7.8, 1.0 Hz, 1H), 5.80 (q, J=4.7 Hz, 1H), 5.02 (d, J=10.1 Hz, 1H), 4.26 (dd, J=10.2, 7.8 Hz, 1H), 3.72 (s, 3H), 2.90 (p, J=7.5 Hz, 1H), 1.64 (q, J=1.2 Hz, 3H), 0.77 (dq, J=7.4, 2.4 Hz, 3H) ppm. ESI-MS m/z calc. 386.09528, found 385.4 (M−1); Retention time: 0.94 minutes.

### Step 7:

Benzyl bromide (200 μL, 1.682 mmol) was added in one portion to a stirred suspension of methyl(2R,3S,4S,5R)-3-(2-hydroxy-3-(trifluoromethyl)phenyl)-4,5-dimethyl-5-(trifluoromethyl)tetrahydrofuran-2-carboxylate (510 mg, 1.083 mmol) and K₂CO₃ (258 mg, 1.867 mmol) in DMF (3 mL) at 55° C. and under a nitrogen atmosphere. The reaction mixture was stirred for 30 min. The mixture was then diluted with EtOAc (30 mL). The organic phase was separated and washed with brine (30 mL) and water (3×15 mL), dried over MgSO₄, filtered and concentrated in vacuo to give an orange oil. Purification by flash chromatography (SiO₂, 0 to 20% EtOAc in heptane) gave methyl(2R,3S,4S,5R)-3-(2-(benzyloxy)-3-(trifluoromethyl)phenyl)-4,5-dimethyl-5-(trifluoromethyl)tetrahydrofuran-2-carboxylate (516 mg, 85%) as a clear, colourless oil. ¹H NMR (500 MHz, Chloroform-d) & 7.61 (dd, J=7.9, 1.6 Hz, 1H), 7.49 (ddd, J=7.4, 6.5, 1.6 Hz, 3H), 7.45-7.41 (m, 2H), 7.41-7.35 (m, 1H), 7.27 (d, J=8.3 Hz, 1H), 5.10 (d, J=11.0 Hz, 1H), 4.93 (d, J=10.5 Hz, 1H), 4.77 (d, J=10.9 Hz, 1H), 4.21 (dd, J=10.6, 7.9 Hz, 1H), 3.73 (s, 3H), 2.62 (p, J=7.6 Hz, 1H), 1.31 (d, J=1.1 Hz, 3H), 0.70 (dq, J=7.4, 2.4 Hz, 3H) ppm. ESI-MS m/z calc. 476.14224, found 475.4 (M−1); Retention time: 1.18 minutes.

### Step 8:

Potassium tert-butoxide (160 mg, 1.426 mmol) was added in one portion to a stirred solution of methyl(2R,3S,4S,5R)-3-(2-(benzyloxy)-3-(trifluoromethyl)phenyl)-4,5-dimethyl-5-(trifluoromethyl)tetrahydrofuran-2-carboxylate (500 mg, 0.892 mmol) in THF (8 mL) at 0° C. and under a nitrogen atmosphere. The reaction mixture was stirred for 15 min at 0° C. then, for 45 min at ambient temperature. The mixture was cooled to 0° C. and a further amount of potassium tert-butoxide (70 mg, 0.6238 mmol) was added under a nitrogen atmosphere. The mixture was warmed to ambient temperature and stirred under a nitrogen atmosphere for an additional 1 h. The mixture was quenched by pouring it over 1 M HCl. The mixture was extracted with DCM (2×10 mL). The combined organic extracts were dried over MgSO₄, filtered, and concentrated in vacuo to give (2R,3S,4S,5R)-3-(2-(benzyloxy)-3-(trifluoromethyl)phenyl)-4,5-dimethyl-5-(trifluoromethyl)tetrahydrofuran-2-carboxylic acid (469 mg, 92%). ¹H NMR (500 MHZ, Chloroform-d) & 7.62 (ddd, J=8.2, 4.1, 1.5 Hz, 1H), 7.53 (dd, J=8.0, 1.6 Hz, 1H), 7.50-7.34 (m, 5H), 7.31-7.26 (m, 1H), 5.15 (d, J=11.2 Hz, 1H), 4.96 (d, J=10.9 Hz, 1H), 4.74 (d, J=11.3 Hz, 1H), 4.09 (dd, J=11.0, 7.8 Hz, 1H), 2.62 (p, J=7.6 Hz, 1H), 1.24 (dd, J=2.4, 1.3 Hz, 3H), 0.69 (dq, J=7.3, 2.4 Hz, 3H) ppm; acid OH not observed. ESI-MS m/z calc. 462.1266, found 461.4 (M−1); Retention time: 0.71 minutes.

### Step 9 and 10:

Oxalyl chloride (70 μL, 0.802 mmol) was added dropwise over 2 min to a stirred solution of (2R,3S,4S,5R)-3-(2-(benzyloxy)-3-(trifluoromethyl)phenyl)-4,5-dimethyl-5-(trifluoromethyl)tetrahydrofuran-2-carboxylic acid (200 mg, 0.350 mmol) and DMF (4 μL, 0.052 mmol) in DCM (3 mL) at ambient temperature and under a nitrogen atmosphere. The reaction mixture was stirred for 20 min. The reaction mixture was concentrated in vacuo to give an orange oil. The residue was taken up in DCM (1.5 mL) and added dropwise to a stirred solution of methyl 4-aminopyridine-2-carboxylate (80 mg, 0.526 mmol) and Et₃N (75 μL, 0.538 mmol) in DCM (1.5 mL) at ambient temperature under a nitrogen atmosphere. The reaction mixture was stirred for 20 min before being quenched with a saturated aqueous sodium bicarbonate solution (5 mL). The aqueous phase was extracted with DCM (3×10 mL). The combined organic extracts were washed with brine (10 mL), dried over MgSO₄, filtered, and concentrated in vacuo to give an orange oil. Purification by flash chromatography (SiO₂, 0 to 50% EtOAc in heptane) gave methyl 4-((2R,3S,4S,5R)-3-(2-(benzyloxy)-3-(trifluoromethyl)phenyl)-4,5-dimethyl-5-(trifluoromethyl)tetrahydrofuran-2-carboxamido) picolinate (140 mg, 60%) as a glassy solid. ESI-MS m/z calc. 596.1746, found 597.5 (M+1)⁺; 595.5 (M−1); Retention time: 1.12 minutes.

Methanolic ammonia (3 mL of 7 M, 21.00 mmol) was added to a solution of methyl 4-((2R,3S,4S,5R)-3-(2-(benzyloxy)-3-(trifluoromethyl)phenyl)-4,5-dimethyl-5-(trifluoromethyl)tetrahydrofuran-2-carboxamido) picolinate (140 mg) in MeOH (2 mL). The reaction mixture was stirred at ambient temperature for 4.5 h. A further portion of methanolic ammonia (3×2 mL of 7 M, 14.00 mmol) was added and the mixture was stirred at room temperature until completion of the reaction. The mixture was concentrated in vacuo to give 4-((2R,3S,4S,5R)-3-(2-(benzyloxy)-3-(trifluoromethyl)phenyl)-4,5-dimethyl-5-(trifluoromethyl)tetrahydrofuran-2-carboxamido) picolinamide (123 mg, 53%) as a glassy solid. ¹H NMR (500 MHZ, Chloroform-d) δ 8.52-8.48 (m, 2H), 8.19 (dd, J=5.5, 2.2 Hz, 1H), 7.92 (d, J=2.2 Hz, 1H), 7.64 (d, J=7.9 Hz, 2H), 7.31 (tt, J=5.4, 3.2 Hz, 3H), 7.25-7.22 (m, 2H), 5.20 (d, J=11.7 Hz, 1H), 4.98 (d, J=11.3 Hz, 1H), 4.69 (d, J=11.7 Hz, 1H), 3.97-3.91 (m, 1H), 2.60 (p, J=7.5 Hz, 1H), 1.22 (s, 3H), 0.74-0.67 (m, 3H) ppm; amides NH and NH₂ not observed. ESI-MS m/z calc. 581.1749, found 582.5 (M+1)⁺; 580.5 (M−1); Retention time: 1.07 minutes.

### Step 11:

4-((2R,3S,4S,5R)-3-(2-(benzyloxy)-3-(trifluoromethyl)phenyl)-4,5-dimethyl-5-(trifluoromethyl)tetrahydrofuran-2-carboxamido) picolinamide (120 mg, 0.182 mmol) and Pd(OH)₂ (202 mg, 0.288 mmol) were suspended in EtOH (5 mL). Hydrogen gas was bubbled through the suspension for 15 min. The reaction mixture was purged with nitrogen, diluted with methanol (10 mL), filtered through a pad of celite and washed with methanol. The filtrate was concentrated in vacuo to give 4-((2R,3S,4S,5R)-3-(2-hydroxy-3-(trifluoromethyl)phenyl)-4,5-dimethyl-5-(trifluoromethyl)tetrahydrofuran-2-carboxamido) picolinamide (94 mg, 87%) as a pale yellow solid. ¹H NMR (500 MHZ, Chloroform-d) δ 8.74 (s, 1H), 8.47 (d, J=5.7 Hz, 1H), 8.19 (dd, J=5.7, 2.1 Hz, 1H), 7.97 (s, 1H), 7.60 (d, J=7.7 Hz, 1H), 7.49-7.45 (m, 1H), 7.13-7.07 (m, 1H), 5.15 (d, J=11.2 Hz, 1H), 4.21 (dd, J=11.2, 7.7 Hz, 1H), 2.94 (p, J=7.5 Hz, 1H), 1.71 (d, J=1.1 Hz, 3H), 0.80 (dt, J=7.3, 2.4 Hz, 3H) ppm; amides NH and NH₂ not observed. ESI-MS m/z calc. 491.12796, found 492.5 (M+1)⁺; 490.4 (M−1); Retention time: 2.93 minutes.

### Step 12:

A solution of ((1r,3r)-3-bromocyclobutoxy)(tert-butyl)dimethylsilane (77 mg, 0.290 mmol) in DMF (1 mL) was added in one portion to a suspension of 4-((2R,3S,4S,5R)-3-(2-hydroxy-3-(trifluoromethyl)phenyl)-4,5-dimethyl-5-(trifluoromethyl)tetrahydrofuran-2-carboxamido) picolinamide (90 mg, 0.152 mmol) and K₂CO₃ (84 mg, 0.608 mmol) in DMF (2 mL) under a nitrogen atmosphere. The reaction mixture was stirred 100° C. under a nitrogen atmosphere for 16 h. A further portion of both K₂CO₃ (84 mg, 0.608 mmol) and ((1r,3r)-3-bromocyclobutoxy)(tert-butyl)dimethylsilane (77 mg, 0.290 mmol) in DMF (0.5 mL) was added and the reaction mixture was heated to 110° C. for an additional 1 h 45 min. The mixture was cooled to ambient temperature and quenched by addition of water (15 mL). The aqueous phase was separated and extracted with EtOAc (3×20 mL). The combined organic extracts were washed with brine (2×20 mL), dried over MgSO₄, filtered, and concentrated in vacuo to give 4-((2R,3S,4S,5R)-3-(2-((1s,3R)-3-((tert-butyldimethylsilyl)oxy)cyclobutoxy)-3-(trifluoromethyl)phenyl)-4,5-dimethyl-5-(trifluoromethyl)tetrahydrofuran-2-carboxamido) picolinamide as an orange oil. ESI-MS m/z calc. 675.2563, found 676.6 (M+1)⁺; 674.5 (M−1); Retention time: 1.27 minutes.

TFA (250 μL, 3.245 mmol) was added to a solution of 4-((2R,3S,4S,5R)-3-(2-((1s,3R)-3-((tert-butyldimethylsilyl)oxy)cyclobutoxy)-3-(trifluoromethyl)phenyl)-4,5-dimethyl-5-(trifluoromethyl)tetrahydrofuran-2-carboxamido) picolinamide in 2-MeTHF (3 mL) and water (100 μL, 5.551 mmol). The reaction mixture was stirred at ambient temperature for 30 min. A second portion of water (100 μL, 5.551 mmol) and TFA (250 μL, 3.245 mmol) was added and the reaction mixture was stirred for a further 1 h. Purification by reverse phase HPLC using a X-bridge C18 column (150×19 mm, 5 mm particle size) from Waters (Gradient: 37.9% to 52.6% acetonitrile in water (supplemented with 0.1% ammonium hydroxide) over 9 min; Flow rate: 19 mL/min; sample dissolved in acetonitrile and injected at 1 mL/min) gave 4-((2R,3S,4S,5R)-3-(2-((1s,3R)-3-hydroxycyclobutoxy)-3-(trifluoromethyl)phenyl)-4,5-dimethyl-5-(trifluoromethyl)tetrahydrofuran-2-carboxamido) picolinamide (212, 5.1 mg, 6%) as an off white solid. ¹H NMR (500 MHZ, Methanol-d₄) δ 8.50 (dd, J=5.5, 0.6 Hz, 1H), 8.29 (dd, J=2.2, 0.7 Hz, 1H), 7.89 (dd, J=5.5, 2.2 Hz, 1H), 7.65 (dd, J=7.9, 1.6 Hz, 1H), 7.58 (dd, J=7.9, 1.6 Hz, 1H), 7.32-7.27 (m, 1H), 5.13 (d, J=10.2 Hz, 1H), 4.38 (dd, J=10.3, 8.1 Hz, 1H), 3.88 (p, J=7.3 Hz, 1H), 3.74 (tt, J=7.7, 6.6 Hz, 1H), 2.96 (dq, J=11.3, 6.4 Hz, 1H), 2.91-2.77 (m, 2H), 2.29 (dq, J=11.7, 7.9 Hz, 2H), 1.69 (d, J=1.3 Hz, 3H), 0.78 (dq, J=7.4, 2.3 Hz, 3H) ppm; amides NH and NH₂ not observed. ESI-MS m/z calc. 561.16986, found 562.6 (M+1)⁺; 560.5 (M−1); Retention time: 3.1 minutes.

### Example 28

### 4-((2R,3S,4S,5R)-3-(3-chloro-2-((1s,3R)-3-hydroxycyclobutoxy)phenyl)-4,5-dimethyl-5-(trifluoromethyl)tetrahydrofuran-2-carboxamido) picolinamide (213)

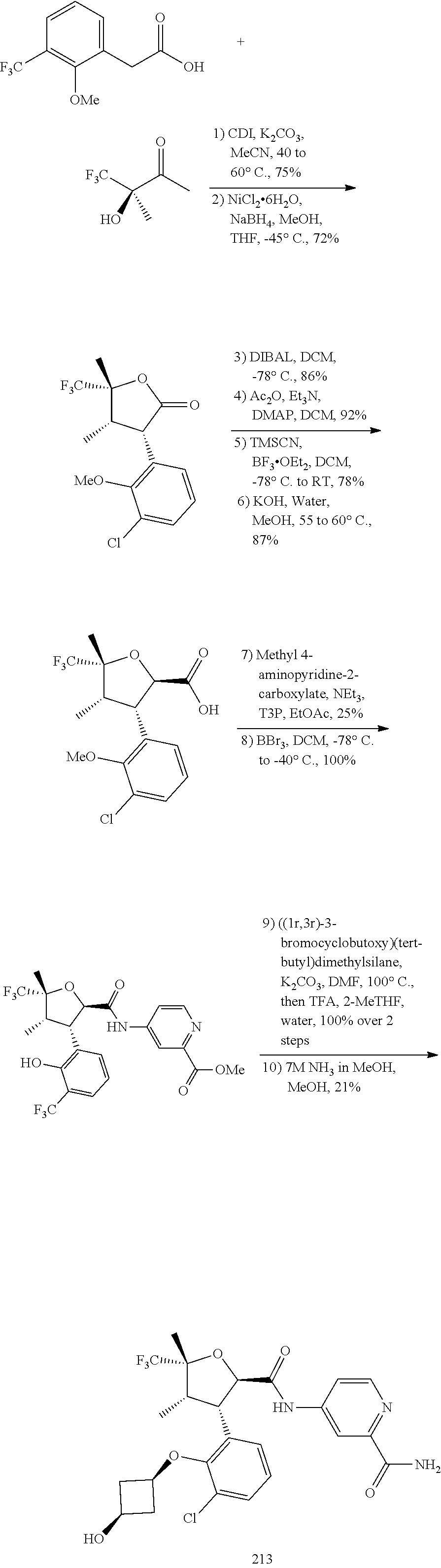

### Step 1:

2-(3-Chloro-2-methoxy-phenyl) acetic acid (Intermediate F, 8.17 g, 39.326 mmol), dissolved in acetonitrile (80 mL) at 40° C., was added over 5 min to a stirred solution of CDI (7.8 g, 48.104 mmol) in acetonitrile (60 mL) at 40° C. The reaction mixture was stirred for 30 min. (R)-4,4,4-trifluoro-3-hydroxy-3-methylbutan-2-one (Intermediate C, 7.6 g, 48.686 mmol) and potassium carbonate (7.34 g, 53.109 mmol) were successively added to the reaction mixture, which was stirred at 60° C. for 16 h. The mixture was filtered and concentrated in vacuo. The residue was dissolved in ethyl acetate (100 mL), washed with 2 N hydrochloric acid (100 mL), with a saturated aqueous sodium bicarbonate solution (2×30 mL), dried over Na₂SO₄, filtered, and concentrated in vacuo to give a brown solid (11.5 g). Purification by flash chromatography (300 g SiO₂, 0 to 90% EtOAc in heptane) gave (R)-3-(3-chloro-2-methoxyphenyl)-4,5-dimethyl-5-(trifluoromethyl) furan-2 (5H)-one (9 g, 75%) as a white solid. ¹H NMR (400 MHZ, Chloroform-d) δ 7.46 (dd, J=7.8, 1.8 Hz, 1H), 7.19 (dd, J=7.8, 1.8 Hz, 1H), 7.14 (t, J=7.8 Hz, 1H), 3.67 (s, 3H), 2.05 (s, 3H), 1.75 (d, J=0.9 Hz, 3H) ppm. ESI-MS m/z calc. 320.0427, found 321.01 (M+1)⁺; 318.9 (M−1); Retention time: 2.74 minutes.

### Step 2:

Nickel dichloride hexahydrate (2.55 g, 10.728 mmol) was added to a stirred and previously degassed solution of (R)-3-(3-chloro-2-methoxyphenyl)-4,5-dimethyl-5-(trifluoromethyl) furan-2 (5H)-one (3.4 g, 10.409 mmol) in MeOH (100 mL) and THF (30 mL) at −40° C. NaBH₄ (2 g, 52.865 mmol) was added portionwise over 30 min and the reaction mixture was stirred at −40° C. Further amounts of NiCl₂ (1×1 eq and 1×0.5 eq) and NaBH₄ (1×5 eq and 1×2.5 eq) were added portionwise. Upon reaction completion, the mixture was quenched by addition of a NH₄Cl solution (30 mL). The reaction mixture was warmed up to ambient temperature and stirred under nitrogen for 15 min. The aqueous phase was separated and extracted with DCM (2×100 mL). The combined organic extracts were dried over Na₂SO₄, filtered, and concentrated in vacuo to give (3S,4S,5R)-3-(3-chloro-2-methoxyphenyl)-4,5-dimethyl-5-(trifluoromethyl)dihydrofuran-2 (3H)-one (2.93 g, 72%) as a colourless oil. ¹H NMR (400 MHZ, Chloroform-d) δ 7.36 (dd, J=7.8, 1.8 Hz, 1H), 7.16 (dd, J=7.8, 1.8 Hz, 1H), 7.07 (t, J=7.8 Hz, 1H), 4.57 (d, J=9.2 Hz, 1H), 3.90 (s, 3H), 2.92 (dd, J=9.4, 7.6 Hz, 1H), 1.72 (d, J=0.9 Hz, 3H), 0.81-0.78 (m, 3H) ppm. ESI-MS m/z calc. 322.0584, found 323.0 (M+1)⁺; Retention time: 2.79 minutes.

### Step 3:

DIBAL (37 mL of a 1 M solution in hexane, 37.000 mmol) was added dropwise to a stirred solution of (3S,4S,5R)-3-(3-chloro-2-methoxyphenyl)-4,5-dimethyl-5-(trifluoromethyl)dihydrofuran-2 (3H)-one (6.84 g, 14.837 mmol) in dichloromethane (100 mL) at −78° C. The reaction mixture was stirred at −78° C. for 1 h. The mixture was quenched by addition of 2 N hydrochloric acid (30 mL) and diluted with dichloromethane (100 mL). The aqueous layer was separated and extracted with dichloromethane (100 mL). The combined organic extracts were washed with 2 N hydrochloric acid (30 mL), dried (Na₂SO₄), filtered and concentrated in vacuo to give (3S,4S,5R)-3-(3-chloro-2-methoxyphenyl)-4,5-dimethyl-5-(trifluoromethyl)tetrahydrofuran-2-ol (5.9 g, 86%) as a colourless oil. 1H NMR (400 MHZ, Chloroform-d) δ 7.32-7.28 (m, 1H), 7.13 (d, J=7.8 Hz, 1H), 7.03 (t, J=8.0 Hz, 1H), 5.83 (d, J=5.0 Hz, 1H), 3.87-3.85 (m, 1H), 3.86 (s, 3H), 2.92-2.81 (m, 1H), 1.64-1.63 (m, 3H), 0.80 (td, J=4.8, 2.6 Hz, 3H) ppm; alcohol OH not observed. ESI-MS m/z calc. 324.074, found 323.0 (M−1); Retention time: 2.55 minutes.

### Step 4:

Ac2O (2.813 g, 2.6 mL, 27.556 mmol) was added to a solution of (3S,4S,5R)-3-(3-chloro-2-methoxyphenyl)-4,5-dimethyl-5-(trifluoromethyl)tetrahydrofuran-2-ol (5.9 g, 13.627 mmol) and triethylamine (2.759 g, 3.8 mL, 27.264 mmol) in dichloromethane (40 mL) at ambient temperature. The reaction mixture was stirred at ambient temperature for 14 h. The mixture was quenched by addition of water (30 mL). The mixture was stirred for 30 min at ambient temperature and diluted with DCM (60 mL). The organic phase was separated, washed with 2 N hydrochloric acid (20 mL), with a saturated aqueous sodium bicarbonate solution (2×20 mL) and brine (10 mL), dried (Na₂SO₄), filtered and concentrated in vacuo to give (3S,4S,5R)-3-(3-chloro-2-methoxyphenyl)-4,5-dimethyl-5-(trifluoromethyl)tetrahydrofuran-2-yl acetate (6.15 g, 92%) as a colourless oil. ¹H NMR (400 MHZ, Chloroform-d) δ 7.32-7.27 (m, 1H), 7.24-7.19 (m, 1H), 7.05-7.01 (m, 1H), 6.58 (d, J=3.2 Hz, 1H), 4.07-4.02 (m, 1H), 3.83 (s, 3H), 2.96-2.85 (m, 1H), 2.08 (s, 3H), 1.62 (d, J=0.9 Hz, 3H), 0.85-0.82 (m, 3H) ppm.

### Step 5:

TMSCN (3.723 g, 5.1 mL, 37.528 mmol) and BF₃· (OEt)₂ (5.405 g, 4.7 mL, 38.083 mmol) were successively added to a stirred solution of (3S,4S,5R)-3-(3-chloro-2-methoxyphenyl)-4,5-dimethyl-5-(trifluoromethyl)tetrahydrofuran-2-yl acetate (6.150 g, 12.576 mmol) in dichloromethane (40 mL) under argon at −78° C. The reaction mixture was stirred at −78° C. for 1 h and at ambient temperature for 14 h. The mixture was poured over a sodium carbonate solution (100 mL) and extracted with dichloromethane (3×30 mL). The combined organic extracts were dried (Na₂SO₄), filtered and concentrated in vacuo to give (2R,3S,4S,5R)-3-(3-chloro-2-methoxyphenyl)-4,5-dimethyl-5-(trifluoromethyl)tetrahydrofuran-2-carbonitrile (4.99 g, 78%) as an orange oil. ¹H NMR (400 MHZ, Chloroform-d) δ 7.37 (dd, J=8.0, 1.6 Hz, 1H), 7.11-7.06 (m, 1H), 6.99-6.97 (m, 1H), 5.02 (d, J=10.1 Hz, 1H), 4.29 (dd, J=10.1, 8.2 Hz, 1H), 3.93 (s, 3H), 2.86 (m, J=7.8 Hz, 1H), 1.63 (s, 3H), 0.81-0.77 (m, 3H) ppm. ¹⁹F NMR (376 MHz, Chloroform-d) δ −74.5 (s, 3F) ppm.

### Step 6:

A stirred mixture of (2R,3S,4S,5R)-3-(3-chloro-2-methoxyphenyl)-4,5-dimethyl-5-(trifluoromethyl)tetrahydrofuran-2-carbonitrile (325 mg, 0.730 mmol) and potassium hydroxide (140 mg, 2.495 mmol) in a mixture of methanol (5 mL) and water (1 mL) was heated at 55° C. for 14 h. A further amount of potassium hydroxide (164 mg, 2.923 mmol) was added to the mixture, which was stirred at 60° C. for an additional 8 h. The methanol was removed in vacuo and the residue was diluted with water (10 mL). The aqueous extracts were extracted with MTBE (5 mL), acidified with 6 N hydrochloric acid (3 mL) and extracted with MTBE (2×10 mL). The combined organic extracts were washed with brine (5 mL), dried over Na₂SO₄, filtered, and concentrated in vacuo to give (2R,3S,4S,5R)-3-(3-chloro-2-methoxyphenyl)-4,5-dimethyl-5-(trifluoromethyl)tetrahydrofuran-2-carboxylic acid (265 mg, 87%) as a colourless oil. ¹H NMR (400 MHZ, Chloroform-d) δ 7.32 (dd, J=8.0, 1.6 Hz, 1H), 7.16 (dd, J=7.8, 1.4 Hz, 1H), 7.05 (t, J=7.8 Hz, 1H), 4.94 (d, J=10.5 Hz, 1H), 4.20 (dd, J=10.5, 7.8 Hz, 1H), 3.85 (d, J=5.5 Hz, 3H), 2.76 (td, J=15.2, 7.6 Hz, 1H), 1.62 (s, 3H), 0.76 (td, J=4.8, 2.4 Hz, 3H) ppm; acid OH not observed. ¹⁹F NMR (376 MHz, Chloroform-d) δ −74.4 (s, 3F) ppm. ESI-MS m/z calc. 352.0689, found 351.0 (M−1); Retention time: 2.51 minutes.

### Step 7:

T3P (240 μL, 50% wt. in EtOAc) was added to a stirred solution of (2R,3S,4S,5R)-3-(3-chloro-2-methoxyphenyl)-4,5-dimethyl-5-(trifluoromethyl)tetrahydrofuran-2-carboxylic acid (105 mg, 0.253 mmol) and Et₃N (140 μL, 1.0 mmol) in EtOAc (1 mL) at ambient temperature. The reaction mixture was stirred for 30 min. A suspension of methyl 4-aminopyridine-2-carboxylate (57 mg, 0.375 mmol) in EtOAc (1 mL) was added in one portion and the reaction mixture was stirred at ambient temperature for 30 min. The mixture was poured over a saturated aqueous NH₄Cl solution (10 mL) and extracted with DCM (3×10 mL). The combined organic extracts were washed with brine (10 mL), dried over MgSO₄, filtered, and concentrated in vacuo to give an orange oil. Purification by flash chromatography (SiO₂, 0 to 50% EtOAc in heptane) gave methyl 4-((2R,3S,4S,5R)-3-(3-chloro-2-methoxyphenyl)-4,5-dimethyl-5-(trifluoromethyl)tetrahydrofuran-2-carboxamido) picolinate (34 mg, 25%) as an off white solid. ESI-MS m/z calc. 486.11694, found 487.5 (M+1)⁺; 485.4 (M−1); Retention time: 0.99 minutes.

### Step 8:

BBr₃ (140 μL of a 1 M solution in heptane, 0.140 mmol) was added dropwise over 2 min to a stirred solution of methyl 4-((2R,3S,4S,5R)-3-(3-chloro-2-methoxyphenyl)-4,5-dimethyl-5-(trifluoromethyl)tetrahydrofuran-2-carboxamido) picolinate (34 mg, 0.063 mmol) in DCM (1 mL) at −78° C. The reaction was stirred for 30 min at −78° C. A further amount of BBr₃ (80 μL of a 1 M solution in heptane, 0.080 mmol) was added dropwise over 2 min and the reaction mixture was stirred at −78° C. under nitrogen for an additional 30 min. The mixture was warmed to −40° C. and stirred under nitrogen for another 45 min. The mixture was quenched by addition of a saturated aqueous NH₄Cl solution (5 mL). The mixture was diluted with DCM (˜5 mL), warmed to ambient temperature and stirred under nitrogen for 2 h. The aqueous phase was separated and extracted with DCM (3×10 mL). The combined organic extracts were dried over MgSO₄, filtered, and concentrated in vacuo to give methyl 4-((2R,3S,4S,5R)-3-(3-chloro-2-hydroxyphenyl)-4,5-dimethyl-5-(trifluoromethyl)tetrahydrofuran-2-carboxamido) picolinate (30 mg, 100%) as an off white solid. ESI-MS m/z calc. 472.1013, found 473.5 (M+1)⁺; 471.4 (M−1); Retention time: 0.89 minutes.

### Step 9:

A solution of ((1r,3r)-3-bromocyclobutoxy)(tert-butyl)dimethylsilane (45 mg, 0.170 mmol) in DMF (0.5 mL) was added in two portions to a suspension of methyl 4-((2R,3S,4S,5R)-3-(3-chloro-2-hydroxyphenyl)-4,5-dimethyl-5-(trifluoromethyl)tetrahydrofuran-2-carboxamido) picolinate (50 mg, 0.08671 mmol) and K₂CO₃ (48 mg, 0.347 mmol) in DMF (1 mL). The reaction mixture was stirred at 100° C. under nitrogen for 16.5 h. The mixture was quenched by addition of water (10 mL) and a saturated aqueous NH₄Cl solution (3 mL). The reaction mixture was extracted with DCM (3×10 mL). The combined organic extracts were dried over MgSO₄, filtered, and concentrated in vacuo to give methyl 4-((2R,3S,4S,5R)-3-(2-((1s,3R)-3-((tert-butyldimethylsilyl)oxy)cyclobutoxy)-3-chlorophenyl)-4,5-dimethyl-5-(trifluoromethyl)tetrahydrofuran-2-carboxamido) picolinate as an orange oil.

TFA (150 μL, 1.947 mmol) was added in one portion to a solution of methyl 4-((2R,3S,4S,5R)-3-(2-((1s,3R)-3-((tert-butyldimethylsilyl)oxy)cyclobutoxy)-3-chlorophenyl)-4,5-dimethyl-5-(trifluoromethyl)tetrahydrofuran-2-carboxamido) picolinate in a mixture of 2-MeTHF (1 mL) and water (50 μL, 2.775 mmol). The reaction mixture was stirred at room temperature for 3 h 45 min. A further amount of water (50 μL, 2.775 mmol) and TFA (150 μL, 1.947 mmol) was added and the reaction mixture was stirred for an additional 75 min. The mixture was quenched by addition of a saturated aqueous sodium bicarbonate solution (10 mL). The mixture was extracted with DCM (3×10 mL). The combined organic extracts were dried over MgSO₄, filtered, and concentrated in vacuo to give methyl 4-((2R,3S,4S,5R)-3-(3-chloro-2-((1s,3R)-3-hydroxycyclobutoxy)phenyl)-4,5-dimethyl-5-(trifluoromethyl)tetrahydrofuran-2-carboxamido) picolinate (48 mg, 100%) as an orange oil, containing residual DMF. ESI-MS m/z calc. 542.1431, found 543.5 (M+1)⁺; 541.5 (M−1); Retention time: 0.91 minutes.

### Step 10:

Methyl 4-((2R,3S,4S,5R)-3-(3-chloro-2-((1s,3R)-3-hydroxycyclobutoxy)phenyl)-4,5-dimethyl-5-(trifluoromethyl)tetrahydrofuran-2-carboxamido) picolinate (46.69 mg, 0.043 mmol) was dissolved in methanolic ammonia (500 μL of 7 M, 3.500 mmol) and the reaction mixture was stirred at ambient temperature for 16 h. The reaction mixture was concentrated in vacuo to give an orange oil. Purification by reverse phase HPLC using a X-bridge C18 column (150×19 mm, 5 mm particle size) from Waters (Gradient: 37.9% to 52.6% acetonitrile in water (supplemented with 0.1% ammonium hydroxide) over 9 min; Flow rate: 19 mL/min; sample dissolved in acetonitrile and injected at 1 mL/min) gave 4-((2R,3S,4S,5R)-3-(3-chloro-2-((1s,3R)-3-hydroxycyclobutoxy)phenyl)-4,5-dimethyl-5-(trifluoromethyl)tetrahydrofuran-2-carboxamido) picolinamide (213, 4.9 mg, 21%) as an off white solid. ¹H NMR (400 MHZ, Methanol-d₄) δ 8.49 (dd, J=5.5, 0.7 Hz, 1H), 8.26 (dd, J=2.2, 0.6 Hz, 1H), 7.89 (dd, J=5.5, 2.2 Hz, 1H), 7.35 (dd, J=8.0, 1.5 Hz, 1H), 7.29 (dd, J=7.9, 1.6 Hz, 1H), 7.09 (t, J=7.9 Hz, 1H), 5.08 (d, J=10.6 Hz, 1H), 4.42 (dd, J=10.6, 7.8 Hz, 1H), 4.06 (tt, J=7.8, 6.7 Hz, 1H), 3.78 (tt, J=7.7, 6.5 Hz, 1H), 2.94-2.76 (m, 3H), 2.30 (dt, J=11.3, 7.8 Hz, 1H), 2.25-2.16 (m, 1H), 1.69 (d, J=1.1 Hz, 3H), 0.80 (dt, J=7.3, 2.4 Hz, 3H) ppm; alcohol OH, amides NH and NH₂ not observed. ESI-MS m/z calc. 527.1435, found 528.5 (M+1)⁺; 526.5 (M−1); Retention time: 2.99 minutes.

### Example 29

### 4-((2R,3S,4S,5R)-3-(3,4-difluoro-2-methoxyphenyl)-4,5-dimethyl-5-(trifluoromethyl)tetrahydrofuran-2-carboxamido)picolinic acid (214), (2R,3S,4S,5R)—N-(2-(1,6-diazaspiro[3.3]heptane-6-carbonyl)pyridin-4-yl)-3-(3,4-difluoro-2-methoxyphenyl)-4,5-dimethyl-5-(trifluoromethyl)tetrahydrofuran-2-carboxamide (215) and (2R,3S,4S,5R)-3-(3,4-difluoro-2-methoxyphenyl)-4,5-dimethyl-N-(2-(1-methyl-1,6-diazaspiro[3.3]heptane-6-carbonyl)pyridin-4-yl)-5-(trifluoromethyl)tetrahydrofuran-2-carboxamide (216)

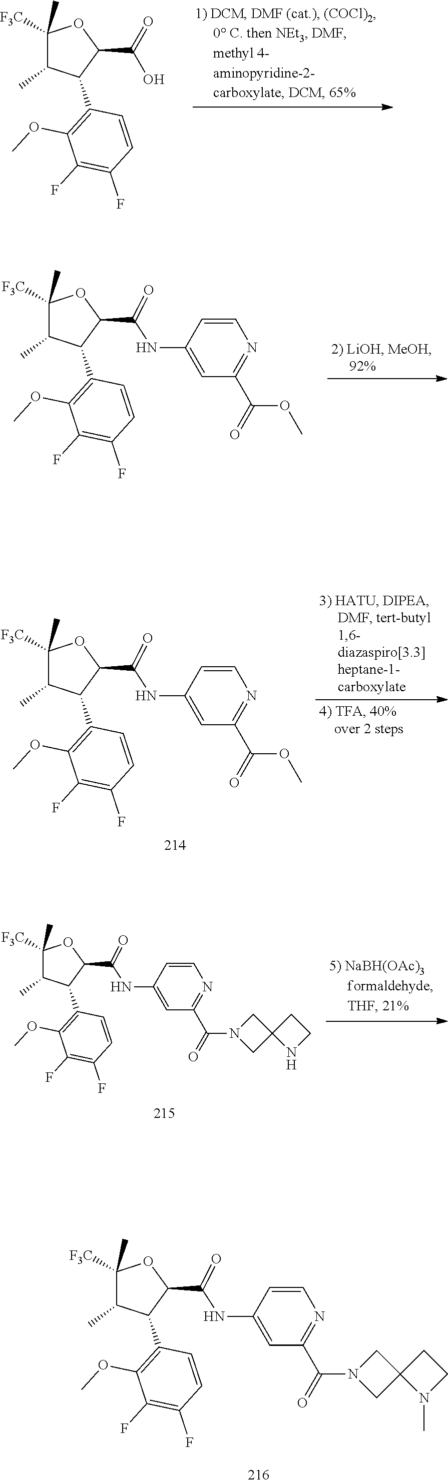

### Step 1:

Oxalyl chloride (1.5 mL, 17.20 mmol) was carefully added to a solution of (2R,3S,4S,5R)-3-(3,4-difluoro-2-methoxyphenyl)-4,5-dimethyl-5-(trifluoromethyl)tetrahydrofuran-2-carboxylic acid (Product of Example 2, Step 5, 2.08 g, 5.460 mmol) and DMF (200 μL, 2.583 mmol) in DCM (50 mL) at 0° C. After stirring the reaction mixture for 30 min, the mixture was concentrated in vacuo. The residue was diluted with DCM (40 mL) and added dropwise to a solution of methyl 4-aminopyridine-2-carboxylate (1 g, 6.572 mmol) and Et₃N (4.0 mL, 28.70 mmol) in DCM (30 mL) at 0° C. The reaction was warmed to ambient temperature after 10 min and stirred for a further 16 h. The reaction mixture was quenched with water (30 mL) and the layers were separated. The aqueous layer was extracted with DCM. The combined organic extracts were dried over MgSO₄, filtered, and concentrated in vacuo to give an orange residue. Purification by flash column chromatography (SiO₂, 0 to 100% EtOAc in heptane) gave methyl 4-((2R,3S,4S,5R)-3-(3,4-difluoro-2-methoxyphenyl)-4,5-dimethyl-5-(trifluoromethyl)tetrahydrofuran-2-carboxamido) picolinate (1.83 g, 65%) as an off-white foam. ¹H NMR (400 MHZ, Chloroform-d) & 8.63 (d, J=5.5 Hz, 1H), 8.58 (s, 1H), 8.08 (dd, J=2.2, 0.6 Hz, 1H), 7.92 (dd, J=5.5, 2.2 Hz, 1H), 7.08 (ddd, J=8.1, 5.6, 2.2 Hz, 1H), 6.91 (td, J=9.2, 7.4 Hz, 1H), 5.03 (d, J=11.1 Hz, 1H), 4.08 (d, J=11.2 Hz, 1H), 4.01 (d, J=3.0 Hz, 6H), 2.76 (p, J=7.7 Hz, 1H), 1.69 (d, J=1.1 Hz, 3H), 0.79 (dq, J=7.4, 2.3 Hz, 3H) ppm. ESI-MS m/z calc. 488.13705, found 489.8 (M+1)⁺; 486.9 (M−1); Retention time: 0.97 minutes.

### Step 2:

2 M LiOH (3.767 mL, 7.534 mmol) was added to a solution of methyl 4-((2R,3S,4S,5R)-3-(3,4-difluoro-2-methoxyphenyl)-4,5-dimethyl-5-(trifluoromethyl)tetrahydrofuran-2-carboxamido) picolinate (1.84 g, 3.767 mmol) in MeOH (10 mL). The reaction mixture was stirred at ambient temperature for 48 h. The mixture was quenched by addition of 1 M HCl (20 mL). The mixture was extracted with EtOAc (×3). The combined organic extracts were dried over MgSO₄, filtered, and concentrated in vacuo to give 4-((2R,3S,4S,5R)-3-(3,4-difluoro-2-methoxyphenyl)-4,5-dimethyl-5-(trifluoromethyl)tetrahydrofuran-2-carboxamido)picolinic acid (1.65 g, 92%) as an off-white solid, which was used without further purification in the next step. ESI-MS m/z calc. 474.1214, found 475.2 (M+1)⁺; 473.1 (M−1); Retention time: 0.66 minutes.

A 30 mg sample was further purified by reverse phase preparative HPLC (basic eluent) to give 4-((2R,3S,4S,5R)-3-(3,4-difluoro-2-methoxyphenyl)-4,5-dimethyl-5-(trifluoromethyl)tetrahydrofuran-2-carboxamido)picolinic acid (214, 11.2 mg, 34%) as a white solid. 1H NMR (500 MHz, DMSO-d₆) δ 10.70 (s, 1H), 8.51 (d, J=5.5 Hz, 1H), 8.26 (s, 1H), 7.80 (d, J=5.6 Hz, 1H), 7.23-7.11 (m, 2H), 5.11 (d, J=10.2 Hz, 1H), 4.25 (dd, J=10.2, 7.7 Hz, 1H), 3.95 (d, J=2.1 Hz, 3H), 2.77 (p, J=7.7 Hz, 1H), 1.61 (s, 3H), 0.80-0.64 (m, 3H) ppm. ESI-MS m/z calc. 474.1214, found 475.2 (M+1)⁺; 473.2 (M−1); Retention time: 2.41 minutes.

### Step 3:

To tert-butyl 1,6-diazaspiro[3.3]heptane-1-carboxylate (42 mg, 0.211 mmol) was added a stock solution of 4-((2R,3S,4S,5R)-3-(3,4-difluoro-2-methoxyphenyl)-4,5-dimethyl-5-(trifluoromethyl)tetrahydrofuran-2-carboxamido)picolinic acid (50 mg, 0.105 mmol), HATU (80 mg, 0.211 mmol) and DIPEA (55 μL, 0.316 mmol) in DMF (1 mL). The mixture was stirred at ambient temperature overnight in a GreenHouse. The mixture was partitioned between water (4 mL) and EtOAc (2 mL). The organic phase was separated by passing through a phase separation cartridge. The organic extracts were concentrated under a stream of nitrogen at 40° C. to give tert-butyl 6-(4-((2R,3S,4S,5R)-3-(3,4-difluoro-2-methoxyphenyl)-4,5-dimethyl-5-(trifluoromethyl)tetrahydrofuran-2-carboxamido) picolinoyl)-1,6-diazaspiro[3.3]heptane-1-carboxylate, which was used in the next step without further purification.

### Step 4:

TFA (1 mL) was added to the crude tert-butyl 6-(4-((2R,3S,4S,5R)-3-(3,4-difluoro-2-methoxyphenyl)-4,5-dimethyl-5-(trifluoromethyl)tetrahydrofuran-2-carboxamido) picolinoyl)-1,6-diazaspiro[3.3]heptane-1-carboxylate and the reaction mixture was stirred at ambient temperature for 5 min. The mixture was concentrated in vacuo. Purification by reverse phase preparative HPLC (acidic eluent) gave (2R,3S,4S,5R)—N-(2-(1,6-diazaspiro[3.3]heptane-6-carbonyl)pyridin-4-yl)-3-(3,4-difluoro-2-methoxyphenyl)-4,5-dimethyl-5-(trifluoromethyl)tetrahydrofuran-2-carboxamide (Trifluoroacetate salt) (215, 40 mg, 52% over 2 steps). ¹H NMR (500 MHz, DMSO-d₆) δ 10.73 (s, 1H), 9.19-8.96 (m, 2H), 8.50 (d, J=5.5 Hz, 1H), 8.27-8.22 (m, 1H), 7.80 (dt, J=5.0, 2.3 Hz, 1H), 7.21-7.10 (m, 2H), 5.07 (dd, J=21.3, 11.2 Hz, 2H), 4.83 (d, J=12.1 Hz, 1H), 4.51 (dd, J=11.9, 1.8 Hz, 1H), 4.32 (dd, J=12.1, 1.8 Hz, 1H), 4.25 (dd, J=10.2, 7.7 Hz, 1H), 3.94 (d, J=2.0 Hz, 3H), 3.81-3.74 (m, 2H), 2.77 (p, J=7.4 Hz, 1H), 2.68 (t, J=8.3 Hz, 2H), 1.60 (s, 3H), 0.73 (d, J=7.2 Hz, 3H) ppm. ESI-MS m/z calc. 554.509, found 555.37 (M+1)⁺; 553.42 (M−1); Retention time: 2.81 minutes

### Step 5:

Formaldehyde (37% aqueous solution, 4 μL, 0.05 mmol) and STAB (26 mg, 0.13 mmol) were successively added to a solution of (2R,3S,4S,5R)—N-(2-(1,6-diazaspiro[3.3]heptane-6-carbonyl)pyridin-4-yl)-3-(3,4-difluoro-2-methoxyphenyl)-4,5-dimethyl-5-(trifluoromethyl)tetrahydrofuran-2-carboxamide (Trifluoroacetate salt) (35 mg, 0.05 mmol) in THF (1 mL). The reaction mixture was stirred at ambient temperature for 16 h. The mixture was partitioned between a saturated aqueous NaHCO₃ solution (1 mL) and EtOAc (1 mL). The organic phase was separated and loaded onto an SCX cartridge (50 mg, prewetted with MeOH). The cartridge was washed with MeOH (1 mL) and then eluted with 7 M NH₃ in MeOH. The basic eluents were concentrated under reduced pressure, redissolved in MeCN and water, and lyophilised overnight to give to give (2R,3S,4S,5R)-3-(3,4-difluoro-2-methoxyphenyl)-4,5-dimethyl-N-(2-(1-methyl-1,6-diazaspiro[3.3]heptane-6-carbonyl)pyridin-4-yl)-5-(trifluoromethyl)tetrahydrofuran-2-carboxamide (216, 6.1 mg, 21%) as a white solid. ¹H NMR (500 MHz, DMSO-d₆) δ 10.71 (s, 1H), 8.46 (d, J=5.5 Hz, 1H), 8.17 (s, 1H), 7.76 (s, 1H), 7.21-7.11 (m, 2H), 5.08 (d, J=10.1 Hz, 1H), 4.68 (d, J=11.2 Hz, 1H), 4.50 (d, J=11.2 Hz, 1H), 4.23 (dd, J=22.1, 10.3 Hz, 2H), 4.00 (d, J=11.2 Hz, 1H), 3.94 (d, J=2.0 Hz, 3H), 3.00 (h, J=6.5 Hz, 2H), 2.77 (t, J=7.6 Hz, 1H), 2.24 (d, J=3.0 Hz, 5H), 1.60 (s, 3H), 0.73 (d, J=7.4 Hz, 3H). ESI-MS m/z calc. 568.536, found 569.38 (M+1)⁺; Retention time: 2.84 minutes.

The following compounds were made using methods similar to those described in Example 29, except that different amine coupling partners were used in the amide coupling step 3. Products from step 3 were purified by reverse phase preparative HPLC and steps 4 and 5 were omitted:

| Cmpd No. | Compound Name | LC/MS | NMR (shifts in ppm) |
|---|---|---|---|
| 217 | 4-((2R,3S,4S,5R)-3-(3,4- | ESI-MS m/z calc. | ¹H NMR (500 MHz, |
| | difluoro-2-methoxyphenyl)-4,5- | 503.1479, found 504.1 | DMSO-d₆) δ 10.76 (s, |
| | dimethyl-5- | (M + 1)⁺; Retention time: | 1H), 8.49 (d, J = 5.7 Hz, |
| | (trifluoromethyl)tetrahydrofuran- | 3.16 minutes | 1H), 7.96 (d, J = 2.1 Hz, |
| | 2-carboxamido)-N-hydroxy-N- | | 1H), 7.73 (dd, J = 5.7, 2.2 |
| | methylpicolinamide | | Hz, 1H), 7.16 (qd, J = |
| | (Trifluoroacetate salt) | | 9.3, 6.5 Hz, 2H), 5.12 (d, |
| | | | J = 10.2 Hz, 1H), 4.26 |
| | | | (dd, J = 10.2, 7.7 Hz, |
| | | | 1H), 3.95 (d, J = 2.0 Hz, |
| | | | 3H), 3.28 (s, 3H), 2.77 (p, |
| | | | J = 7.5 Hz, 1H), 1.60 (s, |
| | | | 3H), 0.73 (dd, J = 7.4, 2.4 |
| | | | Hz, 3H) ppm; alcohol OH |
| | | | not observed. |
| 218 | (2R,3S,4S,5R)-N-(2-(6-oxa-3- | ESI-MS m/z calc. | ¹H NMR (500 MHz, |
| | azabicyclo[3.1.1]heptane-3- | 555.117926, found | DMSO-d₆) δ 10.66 (s, |
| | carbonyl)pyridin-4-yl)-3-(3,4- | 556.3 (M + 1)⁺; 554.1 (M − | 1H), 8.47 (d, J = 5.6 Hz, |
| | difluoro-2-methoxyphenyl)-4,5- | 1)⁻; Retention time: 3.16 | 1H), 7.91 (t, J = 2.2 Hz, |
| | dimethyl-5- | minutes | 1H), 7.67 (dt, J = 5.6, 2.0 |
| | (trifluoromethyl)tetrahydrofuran- | | Hz, 1H), 7.23-7.11 (m, |
| | 2-carboxamide | | 2H), 5.11 (d, J = 10.2 Hz, |
| | | | 1H), 4.67-4.61 (m, 1H), |
| | | | 4.50-4.44 (m, 1H), 4.26 |
| | | | (dd, J = 10.2, 7.7 Hz, |
| | | | 1H), 3.95 (d, J = 2.0 Hz, |
| | | | 3H), 3.90 (dd, J = 13.8, |
| | | | 1.4 Hz, 1H), 3.79 (dd, J = |
| | | | 12.5, 2.2 Hz, 1H), 3.69 |
| | | | (dt, J = 12.7, 1.5 Hz, 1H), |
| | | | 3.59 (dt, J = 13.7, 1.8 Hz, |
| | | | 1H), 3.06 (q, J = 7.4 Hz, |
| | | | 1H), 2.77 (p, J = 7.5 Hz, |
| | | | 1H), 1.80 (dd, J = 9.0, 2.6 |
| | | | Hz, 1H), 1.60 (s, 3H), |
| | | | 0.73 (d, J = 7.6 Hz, 3H) |
| | | | ppm. |
| 219 | (2R,3S,4S,5R)-3-(3,4-difluoro- | ESI-MS m/z calc. | |
| | 2-methoxyphenyl)-N-(2-((S)-3- | 543.17926, found 544.3 | |
| | hydroxypyrrolidine-1- | (M + 1)⁺; 542.2 (M − 1)⁻; | |
| | carbonyl)pyridin-4-yl)-4,5- | Retention time: 3.05 | |
| | dimethyl-5- | minutes | |
| | (trifluoromethyl)tetrahydrofuran- | | |
| | 2-carboxamide | | |
| 220 | (2R,3S,4S,5R)-3-(3,4-difluoro- | ESI-MS m/z calc. | |
| | 2-methoxyphenyl)-N-(2-((R)-3- | 543.17926, found 544.3 | |
| | hydroxypyrrolidine-1- | (M + 1)⁺; 542.2 (M − 1)⁻; | |
| | carbonyl)pyridin-4-yl)-4,5- | Retention time: 3.05 | |
| | dimethyl-5- | minutes | |
| | (trifluoromethyl)tetrahydrofuran- | | |
| | 2-carboxamide | | |
| 221 | 4-((2R,3S,4S,5R)-3-(3,4- | ESI-MS m/z calc. | ¹H NMR (500 MHz, |
| | difluoro-2-methoxyphenyl)-4,5- | 517.16364, found 518.2 | DMSO-d₆) δ |
| | dimethyl-5- | (M + 1)⁺; 516.2 (M − 1)⁻; | |
| | (trifluoromethyl)tetrahydrofuran- | Retention time: 3.15 | |
| | 2-carboxamido)-N-(2- | minutes | |
| | hydroxyethyl)picolinamide | | |

The following compounds were made using methods similar to those described in Example 29, except that different amine coupling partners were used in the amide coupling step 3 and step 5 was omitted:

| Cmpd No. | Compound Name | LC/MS | NMR (shifts in ppm) |
|---|---|---|---|
| 222 | rel-(2R*,3S*,4S*,5R*)-3-(3,4- | ESI-MS m/z calc. | ¹H NMR (500 MHz, |
| | difluoro-2-methoxyphenyl)-4,5- | 556.2109, found 557.3 | DMSO-d₆) δ 10.66 (d, J = |
| | dimethyl-N-(2-(3- | (M + 1)⁺; Retention time: | 7.2 Hz, 1H), 8.46 (t, J = |
| | (methylamino)pyrrolidine-1- | 2.70 minutes | 5.5 Hz, 1H), 7.96 (t, J = |
| | carbonyl)pyridin-4-yl)-5- | | 2.6 Hz, 1H), 7.70 (td, J = |
| | (trifluoromethyl)tetrahydrofuran- | | 5.3, 2.1 Hz, 1H), 7.22- |
| | 2-carboxamide | | 7.11 (m, 2H), 5.10 (d, J = |
| | | | 10.2 Hz, 1H), 4.25 (dd, J = |
| | | | 10.2, 7.7 Hz, 1H), 3.95 |
| | | | (d, J = 2.1 Hz, 3H), 3.75- |
| | | | 3.35 (m, 4H), 3.15 (d, J = |
| | | | 20.2 Hz, 1H), 2.77 (p, J = |
| | | | 7.6 Hz, 1H), 2.34-2.17 |
| | | | (m, 3H), 2.00-1.65 (m, |
| | | | 3H), 1.60 (s, 3H), 0.77- |
| | | | 0.69 (m, 3H) ppm. |
| 223 | rel-(2R*,3S*,4S*,5R*)-3-(3,4- | ESI-MS m/z calc. | ¹H NMR (500 MHz, |
| | difluoro-2-methoxyphenyl)-4,5- | 556.2109, found 557.2 | DMSO-d₆) δ 10.66 (s, |
| | dimethyl-N-(2-(3- | (M + 1)⁺; Retention time: | 1H), 8.46 (t, J = 5.5 Hz, |
| | (methylamino)pyrrolidine-1- | 2.70 minutes | 1H), 7.96 (s, 1H), 7.74- |
| | carbonyl)pyridin-4-yl)-5- | | 7.66 (m, 1H), 7.22-7.10 |
| | (trifluoromethyl)tetrahydrofuran- | | (m, 2H), 5.10 (d, J = 10.2 |
| | 2-carboxamide | | Hz, 1H), 4.28-4.21 (m, |
| | | | 1H), 3.95 (d, J = 2.1 Hz, |
| | | | 3H), 3.76-3.35 (m, 4H), |
| | | | 3.18-3.04 (m, 1H), 2.83- |
| | | | 2.71 (m, 1H), 2.31- |
| | | | 2.17 (m, 3H), 2.00-1.67 |
| | | | (m, 3H), 1.60 (s, 3H), |
| | | | 0.73 (d, J = 7.4 Hz, 3H) |
| | | | ppm. |
| 224 | (2R,3S,4S,5R)-3-(3,4-difluoro- | ESI-MS m/z calc. | |
| | 2-methoxyphenyl)-4,5- | 542.19525, found 543.2 | |
| | dimethyl-N-(2-(piperazine-1- | (M + 1)⁺; 541.2 (M − 1)⁻; | |
| | carbonyl)pyridin-4-yl)-5- | Retention time: 3.04 | |
| | (trifluoromethyl)tetrahydrofuran- | minutes | |
| | 2-carboxamide | | |
| 225 | (2R,3S,4S,5R)-N-(2-(1,6- | ESI-MS m/z calc. | ¹H NMR (500 MHz, |
| | diazaspiro[3.3]heptane-1- | 554.19525, found 555.3 | DMSO-d₆) δ 10.78 (s, |
| | carbonyl)pyridin-4-yl)-3-(3,4- | (M + 1)⁺; 553.2 (M − 1)⁻; | 1H), 8.86 (s, 1H), 8.65 (s, |
| | difluoro-2-methoxyphenyl)-4,5- | Retention time: 3.42 | 1H), 8.51 (d, J = 5.5 Hz, |
| | dimethyl-5- | minutes | 1H), 8.32 (d, J = 2.1 Hz, |
| | (trifluoromethyl)tetrahydrofuran- | | 1H), 7.76 (dd, J = 5.5, 2.2 |
| | 2-carboxamide | | Hz, 1H), 7.21-7.10 (m, |
| | (trifluoroacetate salt) | | 2H), 5.10 (d, J = 10.2 Hz, |
| | | | 1H), 4.66 (s, 2H), 4.47- |
| | | | 4.39 (m, 2H), 4.26 (dd, J = |
| | | | 10.2, 7.7 Hz, 1H), 4.21- |
| | | | 4.13 (m, 2H), 3.95 (d, J = |
| | | | 2.0 Hz, 3H), 2.78 (p, J = |
| | | | 7.6 Hz, 1H), 2.58-2.53 |
| | | | (m, 2H), 1.60 (s, 3H), |
| | | | 0.77-0.68 (m, 3H) ppm. |
| 226 | 4-((2R,3S,4S,5R)-3-(3,4- | ESI-MS m/z calc. | ¹H NMR (500 MHz, |
| | difluoro-2-methoxyphenyl)-4,5- | 592.15482, found 593.3 | DMSO-d₆) δ 10.79 (s, |
| | dimethyl-5- | (M + 1)⁺; 591.2 (M − 1)⁻; | 1H), 9.17 (t, J = 6.2 Hz, |
| | (trifluoromethyl)tetrahydrofuran- | Retention time: 3.41 | 1H), 8.55 (d, J = 5.5 Hz, |
| | 2-carboxamido)-N-(((S)-4,4- | minutes | 1H), 8.34 (d, J = 2.1 Hz, |
| | difluoropyrrolidin-2- | | 1H), 7.87 (dd, J = 5.5, 2.2 |
| | yl)methyl)picolinamide | | Hz, 1H), 7.17 (dd, J = |
| | (Trifluoroacetate salt) | | 10.3, 6.7 Hz, 2H), 5.10 |
| | | | (d, J = 10.1 Hz, 1H), 4.26 |
| | | | (dd, J = 10.3, 7.7 Hz, |
| | | | 1H), 4.02 (s, 1H), 3.95 (d, |
| | | | J = 2.1 Hz, 3H), 3.83 (q, J = |
| | | | 12.4 Hz, 1H), 3.71- |
| | | | 3.63 (m, 3H), 2.82-2.65 |
| | | | (m, 2H), 2.45-2.33 (m, |
| | | | 1H), 1.61 (s, 3H), 0.79- |
| | | | 0.70 (m, 3H) ppm; salt H |
| | | | and amine NH not |
| | | | observed. |
| 227 | (2R,3S,4S,5R)-N-(2-((1S,6R)- | ESI-MS m/z calc. | |
| | 3,8-diazabicyclo[4.2.0]octane- | 568.21090, found | |
| | 8-carbonyl)pyridin-4-yl)-3-(3,4- | 569.33 (M + 1)⁺; | |
| | difluoro-2-methoxyphenyl)-4,5- | Retention time: 2.82 | |
| | dimethyl-5- | minutes | |
| | (trifluoromethyl)tetrahydrofuran- | | |
| | 2-carboxamide | | |
| | (Trifluoroacetate salt) | | |
| 228 | (2R,3S,4S,5R)-N-(2-(2,6- | ESI-MS m/z calc. | |
| | diazaspiro[3.3]heptane-2- | 554.51802, found | |
| | carbonyl)pyridin-4-yl)-3-(3,4- | 555.37 (M + 1)⁺; 554.27 | |
| | difluoro-2-methoxyphenyl)-4,5- | (M − 1)⁻; Retention time: | |
| | dimethyl-5- | 2.76 minutes | |
| | (trifluoromethyl)tetrahydrofuran- | | |
| | 2-carboxamide | | |
| | (Trifluoroacetate salt) | | |
| 229 | 4-((2R,3S,4S,5R)-3-(3,4- | ESI-MS m/z calc. | |
| | difluoro-2-methoxyphenyl)-4,5- | 556.53402, found | |
| | dimethyl-5- | 557.37 (M + 1)⁺; 555.37 | |
| | (trifluoromethyl)tetrahydrofuran- | (M − 1)⁻; Retention time: | |
| | 2-carboxamido)-N-methyl-N- | 2.72 minutes | |
| | ((R)-pyrrolidin-3- | | |
| | yl)picolinamide | | |
| | (Trifluoroacetate salt) | | |
| 230 | (2R,3S,4S,5R)-N-(2-(2,5- | ESI-MS m/z calc. | |
| | diazaspiro[3.4]octane-2- | 568.54502, found | |
| | carbonyl)pyridin-4-yl)-3-(3,4- | 569.33 (M + 1)⁺; 568.13 | |
| | difluoro-2-methoxyphenyl)-4,5- | (M − 1)⁻; Retention time: | |
| | dimethyl-5- | 2.84 minutes | |
| | (trifluoromethyl)tetrahydrofuran- | | |
| | 2-carboxamide | | |
| | (Trifluoroacetate salt) | | |
| 231 | 4-((2R,3S,4S,5R)-3-(3,4- | ESI-MS m/z calc. | |
| | difluoro-2-methoxyphenyl)-4,5- | 530.49602, found | |
| | dimethyl-5- | 531.36 (M + 1)⁺; 530.06 | |
| | (trifluoromethyl)tetrahydrofuran- | (M − 1)⁻; Retention time: | |
| | 2-carboxamido)-N-(2- | 2.83 minutes | |
| | (methylamino)ethyl)picolinami de | | |
| | (Trifluoroacetate salt) | | |
| 232 | (2R,3S,4S,5R)-3-(3,4-difluoro- | ESI-MS m/z calc. | ¹H NMR (500 MHz, |
| | 2-methoxyphenyl)-4,5- | 556.53402, found | DMSO-d₆) δ 10.71 (s, |
| | dimethyl-N-(2-((S)-2- | 557.37 (M + 1)⁺; 555.57 | 1H), 9.02 (s, 1H), 8.58 (s, |
| | methylpiperazine-1- | (M − 1)⁻; Retention time: | 1H), 8.46 (d, J = 5.6 Hz, |
| | carbonyl)pyridin-4-yl)-5- | 2.76 minutes | 1H), 7.96 (s, 1H), 7.64 |
| | (trifluoromethyl)tetrahydrofuran- | | (dd, J = 5.6, 2.1 Hz, 1H), |
| | 2-carboxamide | | 7.21-7.11 (m, 2H), 5.11 |
| | (Trifluoroacetate salt) | | (d, J = 10.2 Hz, 1H), 4.25 |
| | | | (dd, J = 10.3, 7.7 Hz, |
| | | | 1H), 3.95 (d, J = 2.0 Hz, |
| | | | 3H), 3.17 (s, 6H), 2.98 (d, |
| | | | J = 11.6 Hz, 1H), 2.77 (p, |
| | | | J = 7.4 Hz, 1H), 1.60 (s, |
| | | | 3H), 1.32 (d, J = 7.1 Hz, |
| | | | 3H), 0.77-0.69 (m, 3H) |
| | | | ppm. |
| 233 | 4-((2R,3S,4S,5R)-3-(3,4- | ESI-MS m/z calc. | |
| | difluoro-2-methoxyphenyl)-4,5- | 570.22655, found | |
| | dimethyl-5- | 571.43 (M + 1)⁺; 570.48 | |
| | (trifluoromethyl)tetrahydrofuran- | (M − 1)⁻; Retention time: | |
| | 2-carboxamido)-N-(((S)-2- | 2.92 minutes | |
| | methylpyrrolidin-2- | | |
| | yl)methyl)picolinamide | | |
| | (Trifluoroacetate salt) | | |
| 234 | 4-((2R,3S,4S,5R)-3-(3,4- | ESI-MS m/z calc. | |
| | difluoro-2-methoxyphenyl)-4,5- | 544.52302, found | |
| | dimethyl-5- | 545.37 (M + 1)⁺; 543.17 | |
| | (trifluoromethyl)tetrahydrofuran- | (M − 1)⁻; Retention time: | |
| | 2-carboxamido)-N-methyl-N- | 2.77 minutes | |
| | (2- | | |
| | (methylamino)ethyl)picolinami de | | |
| | (Trifluoroacetate salt) | | |
| 235 | (2R,3S,4S,5R)-3-(3,4-difluoro- | ESI-MS m/z calc. | |
| | 2-methoxyphenyl)-4,5- | 556.53402, found 577.37 | |
| | dimethyl-N-(2-((R)-2- | (M + 1)⁺; 555.37 (M − 1)⁻; | |
| | methylpiperazine-1- | Retention time: 2.75 | |
| | carbonyl)pyridin-4-yl)-5- | minutes | |
| | (trifluoromethyl)tetrahydrofuran- | | |
| | 2-carboxamide | | |
| | (Trifluoroacetate salt) | | |
| 236 | N-(azetidin-3-yl)-4- | ESI-MS m/z calc. | |
| | ((2R,3S,4S,5R)-3-(3,4-difluoro- | 528.48002, found | |
| | 2-methoxyphenyl)-4,5- | 529.31 (M + 1)⁺; 527.21 | |
| | dimethyl-5- | (M − 1)⁻; Retention time: | |
| | (trifluoromethyl)tetrahydrofuran- | 2.84 minutes | |
| | 2-carboxamido)picolinamide | | |
| | (Trifluoroacetate salt) | | |
| 237 | (2R,3S,4S,5R)-N-(2-(3,6- | ESI-MS m/z calc. | |
| | diazabicyclo[3.1.1]heptane-6- | 554.51802, found 555.32 | |
| | carbonyl)pyridin-4-yl)-3-(3,4- | (M + 1)⁺; 552.97 (M − 1)⁻; | |
| | difluoro-2-methoxyphenyl)-4,5- | Retention time: 2.84 | |
| | dimethyl-5- | minutes | |
| | (trifluoromethyl)tetrahydrofuran- | | |
| | 2-carboxamide | | |
| | (Trifluoroacetate salt) | | |
| 238 | 4-((2R,3S,4S,5R)-3-(3,4- | ESI-MS m/z calc. | ¹H NMR (500 MHz, |
| | difluoro-2-methoxyphenyl)-4,5- | 542.54702, found | DMSO-d₆) δ 10.77 (s, |
| | dimethyl-5- | 543.37 (M + 1)⁺; 541.12 | 1H), 9.07 (d, J = 7.6 Hz, |
| | (trifluoromethyl)tetrahydrofuran- | (M − 1)⁻; Retention time: | 1H), 8.82-8.62 (m, 2H), |
| | 2-carboxamido)-N-((S)- | 2.84 minutes | 8.53 (d, J = 5.5 Hz, 1H), |
| | pyrrolidin-3-yl)picolinamide | | 8.31 (d, J = 2.1 Hz, 1H), |
| | (Trifluoroacetate salt) | | 7.87 (dd, J = 5.6, 2.2 Hz, |
| | | | 1H), 7.23-7.10 (m, 2H), |
| | | | 5.10 (d, J = 10.3 Hz, 1H), |
| | | | 4.61 (p, J = 6.4 Hz, 1H), |
| | | | 4.26 (dd, J = 10.3, 7.7 Hz, |
| | | | 1H), 3.95 (d, J = 2.0 Hz, |
| | | | 3H), 3.43-3.33 (m, 2H), |
| | | | 3.28-3.18 (m, 2H), 2.78 |
| | | | (p, J = 7.5 Hz, 1H), 2.21 |
| | | | (dq, J = 14.7, 7.5 Hz, |
| | | | 1H), 2.01 (dq, J = 13.1, |
| | | | 6.7 Hz, 1H), 1.61 (s, 3H), |
| | | | 0.78-0.69 (m, 3H) ppm. |
| 239 | (2R,3S,4S,5R)-N-(2-(3,6- | ESI-MS m/z calc. | |
| | diazabicyclo[3.1.1]heptane-3- | 554.51802, found 555.32 | |
| | carbonyl)pyridin-4-yl)-3-(3,4- | (M + 1)⁺; 553.22 (M − 1)⁻; | |
| | difluoro-2-methoxyphenyl)-4,5- | Retention time: 2.74 | |
| | dimethyl-5- | minutes | |
| | (trifluoromethyl)tetrahydrofuran- | | |
| | 2-carboxamide | | |
| | (Trifluoroacetate salt) | | |
| 240 | (2R,3S,4S,5R)-N-(2-((1S,4S)- | ESI-MS m/z calc. | |
| | 2,5-diazabicyclo[2.2.1]heptane- | 554.51802, found | |
| | 2-carbonyl)pyridin-4-yl)-3-(3,4- | 555.32 (M + 1)⁺; 553.32 | |
| | difluoro-2-methoxyphenyl)-4,5- | (M − 1)⁻; Retention time: | |
| | dimethyl-5- | 2.77 minutes | |
| | (trifluoromethyl)tetrahydrofuran- | | |
| | 2-carboxamide | | |
| | (Trifluoroacetate salt) | | |
| 241 | (2R,3S,4S,5R)-3-(3,4-difluoro- | ESI-MS m/z calc. | ¹H NMR (500 MHz, |
| | 2-methoxyphenyl)-N-(2-(3,3- | 570.551, found 571.38 | DMSO-d₆) δ 10.72 (d, J = |
| | dimethylpiperazine-1- | (M + 1)⁺; 569.33 (M − 1)⁻; | 3.5 Hz, 1H), 8.87 (s, 2H), |
| | carbonyl)pyridin-4-yl)-4,5- | Retention time: 2.79 | 8.47 (dd, J = 9.1, 5.6 Hz, |
| | dimethyl-5- | minutes | 1H), 8.01-7.90 (m, 1H), |
| | (trifluoromethyl)tetrahydrofuran- | | 7.73-7.65 (m, 1H), 7.22- |
| | 2-carboxamide | | 7.10 (m, 2H), 5.11 (d, J = |
| | (Trifluoroacetate salt) | | 10.2 Hz, 1H), 4.25 (dd, |
| | | | J = 10.2, 7.7 Hz, 1H), |
| | | | 3.97-3.93 (m, 3H), 3.80 |
| | | | (s, 1H), 3.67 (s, 3H), 3.52 |
| | | | (s, 1H), 3.28 (s, 1H), 3.18 |
| | | | (s, 1H), 2.77 (p, J = 7.5 |
| | | | Hz, 1H), 1.60 (s, 3H), |
| | | | 1.33 (s, 3H), 1.21 (s, 2H), |
| | | | 0.73 (d, J = 7.4 Hz, 3H) |
| | | | ppm. |
| 242 | 4-((2R,3S,4S,5R)-3-(3,4- | ESI-MS m/z calc. | |
| | difluoro-2-methoxyphenyl)-4,5- | 572.5330, found 573.38 | |
| | dimethyl-5- | (M + 1)⁺; 571.43 (M − 1)⁻; | |
| | (trifluoromethyl)tetrahydrofuran- | Retention time: 2.90 | |
| | 2-carboxamido)-N-((3S,4S)- | minutes | |
| | 4-methoxypyrrolidin-3- | | |
| | yl)picolinamide | | |
| | (Trifluoroacetate salt) | | |
| 243 | (2R,3S,4S,5R)-3-(3,4-difluoro- | ESI-MS m/z calc. | |
| | 2-methoxyphenyl)-4,5- | 584.544, found 585.38 | |
| | dimethyl-N-(2-((4aR,7aS)- | (M + 1)⁺; 583.63 (M − 1)⁻; | |
| | octahydropyrrolo[3,4- | Retention time: 2.76 | |
| | b][1,4]oxazine-4- | minutes | |
| | carbonyl)pyridin-4-yl)-5- | | |
| | (trifluoromethyl)tetrahydrofuran- | | |
| | 2-carboxamide | | |
| | (Trifluoroacetate salt) | | |
| 244 | (2R,3S,4S,5R)-N-(2-((1S,5S)- | ESI-MS m/z calc. | |
| | 3,6-diazabicyclo[3.2.2]nonane- | 582.572, found 583.43 | |
| | 3-carbonyl)pyridin-4-yl)-3-(3,4- | (M + 1)⁺; 581.18 (M − 1)⁻; | |
| | difluoro-2-methoxyphenyl)-4,5- | Retention time: 2.77 | |
| | dimethyl-5- | minutes | |
| | (trifluoromethyl)tetrahydrofuran- | | |
| | 2-carboxamide | | |
| | (Trifluoroacetate salt) | | |
| 245 | (2R,3S,4S,5R)-N-(2-(3-oxa-7,9- | ESI-MS m/z calc. | |
| | diazabicyclo[3.3.1]nonane-7- | 584.544, found 585.38 | |
| | carbonyl)pyridin-4-yl)-3-(3,4- | (M + 1)⁺; 583.43 (M − 1)⁻; | |
| | difluoro-2-methoxyphenyl)-4,5- | Retention time: 2.72 | |
| | dimethyl-5- | minutes | |
| | (trifluoromethyl)tetrahydrofuran- | | |
| | 2-carboxamide | | |
| | (Trifluoroacetate salt) | | |
| 246 | 4-((2R,3S,4S,5R)-3-(3,4- | ESI-MS m/z calc. | |
| | difluoro-2-methoxyphenyl)-4,5- | 586.5600, found 587.38 | |
| | dimethyl-5- | (M + 1)⁺; 585.18 (M − 1)⁻; | |
| | (trifluoromethyl)tetrahydrofuran- | Retention time: 2.96 | |
| | 2-carboxamido)-N-((3S,4R)- | minutes | |
| | 4-methoxypiperidin-3- | | |
| | yl)picolinamide | | |
| | (Trifluoroacetate salt) | | |
| 247 | 4-((2R,3S,4S,5R)-3-(3,4- | ESI-MS m/z calc. | |
| | difluoro-2-methoxyphenyl)-4,5- | 586.5600, found 587.43 | |
| | dimethyl-5- | (M + 1)⁺; 586.88 (M − 1)⁻; | |
| | (trifluoromethyl)tetrahydrofuran- | Retention time: 2.80 | |
| | 2-carboxamido)-N-((3S,4S)- | minutes | |
| | 4-methoxypyrrolidin-3-yl)-N- | | |
| | methylpicolinamide | | |
| | (Trifluoroacetate salt) | | |
| 248 | (2R,3S,4S,5R)-3-(3,4-difluoro- | ESI-MS m/z calc. | |
| | 2-methoxyphenyl)-N-(2-((R)-3- | 586.5600, found 587.38 | |
| | (methoxymethyl)piperazine-1- | (M + 1)⁺; 585.33 (M − 1)⁻; | |
| | carbonyl)pyridin-4-yl)-4,5- | Retention time: 2.79 | |
| | dimethyl-5- | minutes | |
| | (trifluoromethyl)tetrahydrofuran- | | |
| | 2-carboxamide | | |
| | (Trifluoroacetate salt) | | |
| 249 | (2R,3S,4S,5R)-3-(3,4-difluoro- | ESI-MS m/z calc. | |
| | 2-methoxyphenyl)-4,5- | 556.5340, found 557.37 | |
| | dimethyl-N-(2-((R)-3- | (M + 1)⁺; 555.32 (M − 1)⁻; | |
| | methylpiperazine-1- | Retention time: 2.75 | |
| | carbonyl)pyridin-4-yl)-5- | minutes | |
| | (trifluoromethyl)tetrahydrofuran- | | |
| | 2-carboxamide | | |
| | (Trifluoroacetate salt) | | |
| 250 | (2R,3S,4S,5R)-3-(3,4-difluoro- | ESI-MS m/z calc. | |
| | 2-methoxyphenyl)-N-(2- | 570.22655, found | |
| | ((2R,5R)-2,5- | 571.43 (M + 1)⁺; 569.18 | |
| | dimethylpiperazine-1- | (M − 1)⁻; Retention time: | |
| | carbonyl)pyridin-4-yl)-4,5- | 2.79 minutes | |
| | dimethyl-5- | | |
| | (trifluoromethyl)tetrahydrofuran- | | |
| | 2-carboxamide | | |
| | (Trifluoroacetate salt) | | |
| 251 | 4-((2R,3S,4S,5R)-3-(3,4- | ESI-MS m/z calc. | |
| | difluoro-2-methoxyphenyl)-4,5- | 570.22655, found | |
| | dimethyl-5- | 571.38 (M + 1)⁺; 570.48 | |
| | (trifluoromethyl)tetrahydrofuran- | (M − 1)⁻; Retention time: | |
| | 2-carboxamido)-N-(((R)- | 2.88 minutes | |
| | piperidin-2- | | |
| | yl)methyl)picolinamide | | |
| | (Trifluoroacetate salt) | | |
| 252 | 4-((2R,3S,4S,5R)-3-(3,4- | ESI-MS m/z calc. | |
| | difluoro-2-methoxyphenyl)-4,5- | 570.22655, found | |
| | dimethyl-5- | 571.43 (M + 1)⁺; 569.58 | |
| | (trifluoromethyl)tetrahydrofuran- | (M − 1)⁻; Retention time: | |
| | 2-carboxamido)-N-((3S,5R)- | 2.95 minutes | |
| | 5-methylpiperidin-3- | | |
| | yl)picolinamide | | |
| | (Trifluoroacetate salt) | | |
| 253 | 4-((2R,3S,4S,5R)-3-(3,4- | ESI-MS m/z calc. | |
| | difluoro-2-methoxyphenyl)-4,5- | 586.56002, found | |
| | dimethyl-5- | 587.43 (M + 1)⁺; 585.18 | |
| | (trifluoromethyl)tetrahydrofuran- | (M − 1)⁻; Retention time: | |
| | 2-carboxamido)-N-((3S,4S)- | 2.96 minutes | |
| | 4-ethoxypyrrolidin-3- | | |
| | yl)picolinamide | | |
| | (Trifluoroacetate salt) | | |
| 254 | (2R,3S,4S,5R)-N-(2-((1R,4R)- | ESI-MS m/z calc. | |
| | 2,5-diazabicyclo[2.2.1]heptane- | 584.509, found 555.32 | |
| | 2-carbonyl)pyridin-4-yl)-3-(3,4- | (M + 1)⁺; 553.37 (M − 1)⁻; | |
| | difluoro-2-methoxyphenyl)-4,5- | Retention time: 2.77 | |
| | dimethyl-5- | minutes | |
| | (trifluoromethyl)tetrahydrofuran- | | |
| | 2-carboxamide | | |
| | (Trifluoroacetate salt) | | |
| 255 | (2R,3S,4S,5R)-N-(2-((3S,4R)-3- | ESI-MS m/z calc. | ¹H NMR (500 MHz, |
| | amino-4-fluoropyrrolidine-1- | 560.4974, found 561.52 | DMSO-d₆) δ 10.74 (d, J = |
| | carbonyl)pyridin-4-yl)-3-(3,4- | (M + 1)⁺; 560.87 (M − 1)⁻; | 9.5 Hz, 1H), 8.50 (dd, J = |
| | difluoro-2-methoxyphenyl)-4,5- | Retention time: 2.74 | 7.2, 5.6 Hz, 1H), 8.42 (d, |
| | dimethyl-5- | minutes | J = 25.8 Hz, 2H), 8.13 |
| | (trifluoromethyl)tetrahydrofuran- | | (dd, J = 6.5, 2.1 Hz, 1H), |
| | 2-carboxamide | | 7.76 (ddd, J = 18.9, 5.6, |
| | (Trifluoroacetate salt) | | 2.1 Hz, 1H), 7.16 (dd, J = |
| | | | 10.4, 6.5 Hz, 2H), 5.35 |
| | | | (dd, J = 53.8, 25.4 Hz, |
| | | | 1H), 5.10 (d, J = 10.2 Hz, |
| | | | 1H), 4.19-3.81 (m, 9H), |
| | | | 3.53 (t, J = 10.9 Hz, 1H), |
| | | | 2.77 (p, J = 7.4 Hz, 1H), |
| | | | 1.60 (s, 3H), 0.73 (d, J = |
| | | | 7.3 Hz, 3H) ppm. |
| 256 | (2R,3S,4S,5R)-3-(3,4-difluoro- | ESI-MS m/z calc. | |
| | 2-methoxyphenyl)-N-(2- | 586.551, found 587.53 | |
| | ((3S,4S)-3-methoxy-4- | (M + 1)⁺; 585.58 (M − 1)⁻; | |
| | (methylamino)pyrrolidine-1- | Retention time: 2.80 | |
| | carbonyl)pyridin-4-yl)-4,5- | minutes | |
| | dimethyl-5- | | |
| | (trifluoromethyl)tetrahydrofuran- | | |
| | 2-carboxamide | | |
| | (Trifluoroacetate salt) | | |
| 257 | (2R,3S,4S,5R)-N-(2-((3S,4R)-3- | ESI-MS m/z calc. | |
| | amino-4-methoxypyrrolidine-1- | 572.524, found 573.53 | |
| | carbonyl)pyridin-4-yl)-3-(3,4- | (M + 1)⁺; 571.23 (M − 1)⁻; | |
| | difluoro-2-methoxyphenyl)-4,5- | Retention time: 2.75 | |
| | dimethyl-5- | minutes | |
| | (trifluoromethyl)tetrahydrofuran- | | |
| | 2-carboxamide | | |
| | (Trifluoroacetate salt) | | |
| 258 | (2R,3S,4S,5R)-N-(2-((3R,4R)-3- | ESI-MS m/z calc. . | |
| | amino-4-fluoropyrrolidine-1- | 560.489, found 561.47 | |
| | carbonyl)pyridin-4-yl)-3-(3,4- | (M + 1)⁺; 559.37 (M − 1)⁻; | |
| | difluoro-2-methoxyphenyl)-4,5- | Retention time: 2.79 | |
| | dimethyl-5- | minutes | |
| | (trifluoromethyl)tetrahydrofuran- | | |
| | 2-carboxamide | | |
| | (Trifluoroacetate salt) | | |
| 259 | (2R,3S,4S,5R)-N-(2-((3R,4S)-3- | ESI-MS m/z calc. | |
| | amino-4-fluoropyrrolidine-1- | 560.489, found 561.42 | |
| | carbonyl)pyridin-4-yl)-3-(3,4- | (M + 1)⁺; 559.17 (M − 1)⁻; | |
| | difluoro-2-methoxyphenyl)-4,5- | Retention time: 2.74 | |
| | dimethyl-5- | minutes | |
| | (trifluoromethyl)tetrahydrofuran- | | |
| | 2-carboxamide | | |
| | (Trifluoroacetate salt) | | |
| 260 | (2R,3S,4S,5R)-N-(2-((3S,4S)-3- | ESI-MS m/z calc. | |
| | amino-4-fluoropyrrolidine-1- | 560.489, found 561.42 | |
| | carbonyl)pyridin-4-yl)-3-(3,4- | (M + 1)⁺; 559.32 (M − 1)⁻; | |
| | difluoro-2-methoxyphenyl)-4,5- | Retention time: 2.80 | |
| | dimethyl-5- | minutes | |
| | (trifluoromethyl)tetrahydrofuran- | | |
| | 2-carboxamide | | |
| | (Trifluoroacetate salt) | | |
| 261 | (2R,3S,4S,5R)-N-(2-((3R,4S)-3- | ESI-MS m/z calc. | ¹H NMR (500 MHz, |
| | amino-4-methoxypyrrolidine-1- | 572.524, found 573.48 | DMSO-d₆) δ 10.73 (d, J = |
| | carbonyl)pyridin-4-yl)-3-(3,4- | (M + 1)⁺; 571.28 (M − 1)⁻; | 6.0 Hz, 1H), 8.49 (t, J = |
| | difluoro-2-methoxyphenyl)-4,5- | Retention time: 2.75 | 5.7 Hz, 1H), 8.18-8.00 |
| | dimethyl-5- | minutes | (m, 3H), 7.73 (ddd, J = |
| | (trifluoromethyl)tetrahydrofuran- | | 11.4, 5.9, 2.2 Hz, 1H), |
| | 2-carboxamide | | 7.16 (dd, J = 11.9, 6.6 Hz, |
| | (Trifluoroacetate salt) | | 2H), 5.10 (d, J = 10.2 Hz, |
| | | | 1H), 4.25 (dd, J = 10.2, |
| | | | 7.7 Hz, 1H), 4.09-3.99 |
| | | | (m, 2H), 3.96-3.78 (m, |
| | | | 7H), 3.34 (d, J = 46.4 Hz, |
| | | | 4H), 2.77 (p, J = 7.3 Hz, |
| | | | 1H), 1.60 (s, 3H), 0.78- |
| | | | 0.67 (m, 3H) ppm. |
| 262 | (2R,3S,4S,5R)-N-(2-((3S,4S)-3- | ESI-MS m/z calc. | ¹H NMR (500 MHz, |
| | amino-4-methoxypyrrolidine-1- | 572.2058, found 573.40 | DMSO-d₆) δ 10.68 (s, |
| | carbonyl)pyridin-4-yl)-3-(3,4- | (M + 1)⁺; 571.4 (M − 1)⁻; | 1H), 8.47 (t, J = 5.8 Hz, |
| | difluoro-2-methoxyphenyl)-4,5- | Retention time: 3.08 | 1H), 7.98 (s, 1H), 7.71 (s, |
| | dimethyl-5- | minutes | 1H), 7.17 (dd, J = 18.4, |
| | (trifluoromethyl)tetrahydrofuran- | | 8.0 Hz, 2H), 5.10 (d, J = |
| | 2-carboxamide | | 10.2 Hz, 1H), 4.29-4.22 |
| | | | (m, 1H), 3.95 (d, J = 2.0 |
| | | | Hz, 3H), 3.91-3.37 (m, |
| | | | 5H), 3.28-3.18 (m, 4H), |
| | | | 2.83-2.69 (m, 1H), 1.60 |
| | | | (s, 5H), 0.73 (d, J = 7.3 |
| | | | Hz, 3H) ppm. |
| 263 | (2R,3S,4S,5R)-N-(2-((3R,4R)-3- | ESI-MS m/z calc. | ¹H NMR (500 MHz, |
| | amino-4-methoxypyrrolidine-1- | 572.2058, found 573.4 | DMSO-d₆) δ 10.68 (s, |
| | carbonyl)pyridin-4-yl)-3-(3,4- | (M + 1)⁺; 571.43 (M − 1)⁻; | 1H), 8.46 (t, J = 5.9 Hz, |
| | difluoro-2-methoxyphenyl)-4,5- | Retention time: 3.09 | 1H), 7.98 (s, 1H), 7.71 (s, |
| | dimethyl-5- | minutes | 1H), 7.22-7.10 (m, 2H), |
| | (trifluoromethyl)tetrahydrofuran- | | 5.10 (d, J = 10.2 Hz, 1H), |
| | 2-carboxamide | | 4.29-4.21 (m, 1H), 3.95 |
| | | | (d, J = 2.1 Hz, 3H), 3.90- |
| | | | 3.47 (m, 4H), 3.41-3.31 |
| | | | (m, 2H), 3.28-3.19 (m, |
| | | | 3H), 2.77 (p, J = 7.7 Hz, |
| | | | 1H), 1.76-1.48 (m, 5H), |
| | | | 0.78-0.66 (m, 3H) ppm. |

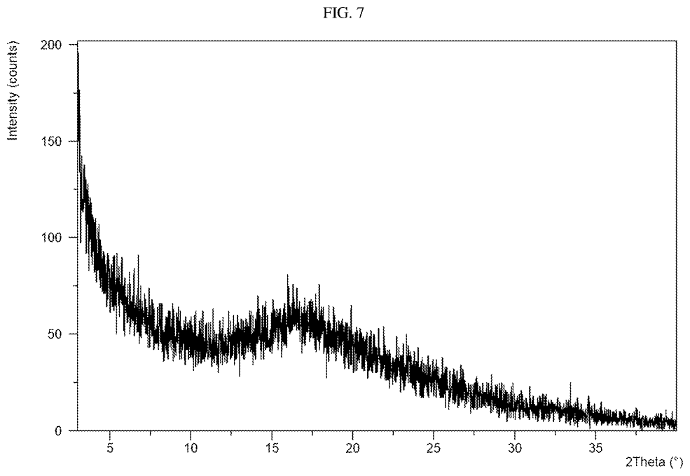

Compound 224 was analyzed by X-ray powder diffraction and determined to be amorphous (see FIG. 7).

The following compounds were made using methods similar to those described in Example 29, except that different amine coupling partners were used in the amide coupling step 3. In the case of compounds 263 and 264, the diastereoisomers from step 3 were further separated by chiral SFC using a Lux Cellulose-2 column, 5 μm particle size, 25 cm×21.2 mm from Phenomenex, Inc. (Mobile phase: 55% methanol: acetonitrile (in a 1:1 ratio, supplemented with 0.2% DMPA), 45% CO₂; System pressure: 100 bar) on a Minigram SFC instrument from Berger Instruments before proceeding with step 4 and 5. In the case of compounds 293 and 294, the diastereoisomers from step 3 were further separated by chiral SFC using a Chiralpak IB column, 5 μm particle size, 25 cm×20 mm from Phenomenex, Inc. (Mobile phase: 7% methanol (supplemented with 20 mM NH₃), 93% CO₂; System pressure: 100 bar) on a Prep-100 SFC instrument from Waters before proceeding with step 4 and 5 to give:

| Cmpd No. | Compound Name | LC/MS | NMR (shifts in ppm) |
|---|---|---|---|
| 264 | rel-(2R*,3S*,4S*,5R*)-3-(3,4- | ESI-MS m/z calc. | ¹H NMR (500 MHz, |
| | difluoro-2-methoxyphenyl)-N- | 570.22656, found 571.3 | DMSO-d₆) δ 10.66 (d, J = |
| | (2-(3- | (M + 1)⁺; 569.2 (M − 1)⁻; | 3.0 Hz, 1H), 8.46 (dd, J = |
| | (dimethylamino)pyrrolidine-1- | Retention time: 3.23 | 10.2, 5.6 Hz, 1H), 7.98 |
| | carbonyl)pyridin-4-yl)-4,5- | minutes | (d, J = 2.1 Hz, 1H), 7.70 |
| | dimethyl-5- | | (dd, J = 5.5, 2.1 Hz, 1H), |
| | (trifluoromethyl)tetrahydrofuran- | | 7.21-7.10 (m, 2H), 5.10 |
| | 2-carboxamide | | (d, J = 10.2 Hz, 1H), 4.25 |
| | (Precursor was the first eluting | | (dd, J = 10.2, 7.7 Hz, |
| | isomer by SFC on a Lux | | 1H), 3.95 (d, J = 2.1 Hz, |
| | Cellulose-2 column, rt = 2.80 | | 3H), 3.83-3.54 (m, 2H), |
| | min) | | 3.48-3.35 (m, 1H), 3.22 |
| | | | (s, 1H), 2.81-2.65 (m, |
| | | | 2H), 2.25-1.95 (m, 7H), |
| | | | 1.77-1.65 (m, 1H), 1.60 |
| | | | (s, 3H), 0.78-0.66 (m, |
| | | | 3H) ppm. |
| 265 | rel-(2R*,3S,*4S*,5R*)-3-(3,4- | ESI-MS m/z calc. | ¹H NMR (500 MHz, |
| | difluoro-2-methoxyphenyl)-N- | 570.22656, found 571.3 | DMSO-d₆) δ 10.67 (s, |
| | (2-(3- | (M + 1)⁺; 569.2 (M − 1)⁻; | 1H), 8.46 (dd, J = 9.8, 5.6 |
| | (dimethylamino)pyrrolidine-1- | Retention time: 3.23 | Hz, 1H), 7.98 (s, 1H), |
| | carbonyl)pyridin-4-yl)-4,5- | minutes | 7.73-7.66 (m, 1H), 7.21- |
| | dimethyl-5- | | 7.11 (m, 2H), 5.10 (d, J = |
| | (trifluoromethyl)tetrahydrofuran- | | 10.2 Hz, 1H), 4.28- |
| | 2-carboxamide | | 4.19 (m, 1H), 3.95 (d, J = |
| | (Precursor was the second | | 2.0 Hz, 3H), 3.85-3.37 |
| | eluting isomer by SFC on a Lux | | (m, 4H), 2.77 (p, J = 7.4 |
| | Cellulose-2 column, rt = 3.71 | | Hz, 1H), 2.30-1.97 (m, |
| | min) | | 8H), 1.80-1.66 (m, 1H), |
| | | | 1.60 (s, 3H), 0.73 (d, J = |
| | | | 7.5 Hz, 3H) ppm. |
| 266 | (2R,3S,4S,5R)-3-(3,4-difluoro- | ESI-MS m/z calc. | ¹H NMR (500 MHz, |
| | 2-methoxyphenyl)-4,5- | 568.536, found 569.3 | DMSO-d₆) δ 10.79 (d, J = |
| | dimethyl-N-(2-(6-methyl-1,6- | (M + 1)⁺; 567.2 (M − 1)⁻; | 36.3 Hz, 1H), 9.54 (d, J = |
| | diazaspiro[3.3]heptane-1- | Retention time: 3.56 | 32.9 Hz, 1H), 8.51 (t, J = |
| | carbonyl)pyridin-4-yl)-5- | minutes | 6.1 Hz, 1H), 8.37 (dd, J = |
| | (trifluoromethyl)tetrahydrofuran- | | 11.5, 2.2 Hz, 1H), 7.74 |
| | 2-carboxamide | | (ddd, J = 14.2, 5.5, 2.2 |
| | (Trifluoroacetate salt) | | Hz, 1H), 7.22-7.08 (m, |
| | | | 2H), 5.11 (dd, J = 10.1, |
| | | | 2.4 Hz, 1H), 4.86-4.77 |
| | | | (m, 1H), 4.66 (dd, J = |
| | | | 12.1, 4.9 Hz, 1H), 4.52- |
| | | | 4.39 (m, 3H), 4.30-4.19 |
| | | | (m, 2H), 3.95 (d, J = 2.0 |
| | | | Hz, 3H), 3.07 (d, J = 4.9 |
| | | | Hz, 1H), 2.85 (d, J = 5.2 |
| | | | Hz, 2H), 2.82-2.74 (m, |
| | | | 1H), 2.59-2.52 (m, 2H), |
| | | | 1.60 (d, J = 3.6 Hz, 3H), |
| | | | 0.74 (d, J = 7.4 Hz, 3H) |
| | | | ppm. |
| 267 | N-(((S)-4,4-difluoro-1- | ESI-MS m/z calc. | ¹H NMR (500 MHz, |
| | methylpyrrolidin-2-yl)methyl)- | 606.532, found | DMSO-d₆) δ 10.75 (s, |
| | 4-((2R,3S,4S,5R)-3-(3,4- | 607.4(M + 1)⁺; Retention | 1H), 8.53 (d, J = 5.3 Hz, |
| | difluoro-2-methoxyphenyl)-4,5- | time: 3.01 minutes | 1H), 8.31 (s, 1H), 7.86 |
| | dimethyl-5- | | (dd, J = 5.6, 2.1 Hz, 1H), |
| | (trifluoromethyl)tetrahydrofuran- | | 7.21-7.12 (m, 2H), 5.10 |
| | 2-carboxamido)picolinamide | | (d, J = 10.2 Hz, 1H), 4.26 |
| | (Trifluoroacetate salt) | | (dd, J = 10.2, 7.7 Hz, |
| | | | 1H), 3.95 (d, J = 2.0 Hz, |
| | | | 3H), 3.70-3.47 (m, |
| | | | 11H), 2.82-2.74 (m, |
| | | | 1H), 1.61 (s, 3H), 0.74 (d, |
| | | | J = 7.0 Hz, 3H) ppm; 1 H |
| | | | coincides with water |
| | | | signal. |
| 268 | (2R,3S,4S,5R)-3-(3,4-difluoro- | ESI-MS m/z calc. | ¹H NMR (500 MHz, |
| | 2-methoxyphenyl)-4,5- | 568.536, found 569.43 | DMSO-d₆) δ 10.68 (s, |
| | dimethyl-N-(2-(6-methyl-2,6- | (M + 1)⁺; 568.43 (M − 1)⁻; | 1H), 8.47 (d, J = 5.5 Hz, |
| | diazaspiro[3.3]heptane-2- | Retention time: 2.79 | 1H), 8.17 (d, J = 2.2 Hz, |
| | carbonyl)pyridin-4-yl)-5- | minutes | 1H), 7.77 (dd, J = 5.5, 2.2 |
| | (trifluoromethyl)tetrahydrofuran- | | Hz, 1H), 7.22-7.11 (m, |
| | 2-carboxamide | | 2H), 5.08 (d, J = 10.2 Hz, |
| | | | 1H), 4.60-4.55 (m, 2H), |
| | | | 4.28-4.21 (m, 1H), 4.10 |
| | | | (s, 2H), 3.94 (d, J = 2.1 |
| | | | Hz, 3H), 3.22 (q, J = 7.4 |
| | | | Hz, 4H), 2.77 (t, J = 7.6 |
| | | | Hz, 1H), 2.15 (s, 3H), |
| | | | 1.60 (s, 3H), 0.73 (d, J = |
| | | | 7.3 Hz, 3H) ppm. |
| 269 | 4-((2R,3S,4S,5R)-3-(3,4- | ESI-MS m/z calc. | |
| | difluoro-2-methoxyphenyl)-4,5- | 570.551, found 571.43 | |
| | dimethyl-5- | (M + 1)⁺; 569.58 (M − 1)⁻; | |
| | (trifluoromethyl)tetrahydrofuran- | Retention time: 2.796 | |
| | 2-carboxamido)-N-methyl-N- | minutes | |
| | ((R)-1-methylpyrrolidin-3- | | |
| | yl)picolinamide | | |
| 270 | (2R,3S,4S,5R)-3-(3,4-difluoro- | ESI-MS m/z calc. | |
| | 2-methoxyphenyl)-4,5- | 582.562 found 583.43 | |
| | dimethyl-N-(2-(5-methyl-2,5- | (M + 1)⁺; 581.18 (M − 1)⁻; | |
| | diazaspiro[3.4]octane-2- | Retention time: 2.87 | |
| | carbonyl)pyridin-4-yl)-5- | minutes | |
| | (trifluoromethyl)tetrahydrofuran- | | |
| | 2-carboxamide | | |
| 271 | 4-((2R,3S,4S,5R)-3-(3,4- | ESI-MS m/z calc. | |
| | difluoro-2-methoxyphenyl)-4,5- | 544.514 found 545.42 | |
| | dimethyl-5- | (M + 1)⁺; 545.57 (M − 1)⁻; | |
| | (trifluoromethyl)tetrahydrofuran- | Retention time: 2.85 | |
| | 2-carboxamido)-N-(2- | minutes | |
| | (dimethylamino)ethyl)picolinamide | | |
| 272 | (2R,3S,4S,5R)-3-(3,4-difluoro- | ESI-MS m/z calc. | |
| | 2-methoxyphenyl)-N-(2-((S)- | 570.5610 found 571.38 | |
| | 2,4-dimethylpiperazine-1- | (M + 1)⁺; 569.58 (M − 1)⁻; | |
| | carbonyl)pyridin-4-yl)-4,5- | Retention time: 2.77 | |
| | dimethyl-5- | minutes | |
| | (trifluoromethyl)tetrahydrofuran- | | |
| | 2-carboxamide | | |
| 273 | 4-((2R,3S,4S,5R)-3-(3,4- | ESI-MS m/z calc. | |
| | difluoro-2-methoxyphenyl)-4,5- | 558.5500 found 559.42 | |
| | dimethyl-5- | (M + 1)⁺; 557.32 (M − 1)⁻; | |
| | (trifluoromethyl)tetrahydrofuran- | Retention time: 2.79 | |
| | 2-carboxamido)-N-(2- | minutes | |
| | (dimethylamino)ethyl)-N- | | |
| | methylpicolinamide | | |
| 274 | (2R,3S,4S,5R)-3-(3,4-difluoro- | ESI-MS m/z calc. | |
| | 2-methoxyphenyl)-N-(2-((R)- | 570.5610 found 571.43 | |
| | 2,4-dimethylpiperazine-1- | (M + 1)⁺; 570.08 (M − 1)⁻; | |
| | carbonyl)pyridin-4-yl)-4,5- | Retention time: 2.78 | |
| | dimethyl-5- | minutes | |
| | (trifluoromethyl)tetrahydrofuran- | | |
| | 2-carboxamide | | |
| 275 | 4-((2R,3S,4S,5R)-3-(3,4- | ESI-MS m/z calc. | |
| | difluoro-2-methoxyphenyl)-4,5- | 542.498 found 543.3 | |
| | dimethyl-5- | (M + 1)⁺; Retention time: | |
| | (trifluoromethyl)tetrahydrofuran- | 2.87 minutes | |
| | 2-carboxamido)-N-(1- | | |
| | methylazetidin-3- | | |
| | yl)picolinamide | | |
| 276 | (2R,3S,4S,5R)-3-(3,4-difluoro- | ESI-MS m/z calc. | ¹H NMR (500 MHz, |
| | 2-methoxyphenyl)-4,5- | 568.536 found 569.33 | DMSO-d₆) δ 10.70 (s, |
| | dimethyl-N-(2-(3-methyl-3,6- | (M + 1)⁺; 567.08 (M − 1)⁻; | 1H), 8.46 (d, J = 5.4 Hz, |
| | diazabicyclo[3.1.1]heptane-6- | Retention time: 2.88 | 1H), 8.19 (d, J = 6.1 Hz, |
| | carbonyl)pyridin-4-yl)-5- | minutes | 1H), 7.76 (s, 1H), 7.23- |
| | (trifluoromethyl)tetrahydrofuran- | | 7.13 (m, 2H), 5.09 (d, J = |
| | 2-carboxamide | | 10.3 Hz, 1H), 5.07-5.00 |
| | | | (m, 1H), 4.41-4.33 (m, |
| | | | 1H), 4.25 (dd, J = 10.3, |
| | | | 7.7 Hz, 1H), 3.95 (d, J = |
| | | | 2.0 Hz, 3H), 2.96 (dd, J = |
| | | | 10.6, 2.7 Hz, 1H), 2.90 |
| | | | (d, J = 2.3 Hz, 2H), 2.78 |
| | | | (p, J = 7.4 Hz, 1H), 2.68- |
| | | | 2.63 (m, 1H), 2.48-2.45 |
| | | | (m, 1H), 2.23 (d, J = 1.3 |
| | | | Hz, 3H), 1.90 (d, J = 7.7 |
| | | | Hz, 1H), 1.61 (s, 3H), |
| | | | 0.74 (d, J = 7.3 Hz, 3H) |
| | | | ppm. |
| 277 | 4-((2R,3S,4S,5R)-3-(3,4- | ESI-MS m/z calc. | |
| | difluoro-2-methoxyphenyl)-4,5- | 556.525 found 557.37 | |
| | dimethyl-5- | (M + 1)⁺; 555.42 (M − 1)⁻; | |
| | (trifluoromethyl)tetrahydrofuran- | Retention time: 2.87 | |
| | 2-carboxamido)-N-((S)-1- | minutes | |
| | methylpyrrolidin-3- | | |
| | yl)picolinamide | | |
| 278 | (2R,3S,4S,5R)-3-(3,4-difluoro- | ESI-MS m/z calc. | |
| | 2-methoxyphenyl)-4,5- | 568.536 found 569.33 | |
| | dimethyl-N-(2-(6-methyl-3,6- | (M + 1)⁺; 567.58 (M − 1)⁻; | |
| | diazabicyclo[3.1.1 ]heptane-3- | Retention time: 2.77 | |
| | carbonyl)pyridin-4-yl)-5- | minutes | |
| | (trifluoromethyl)tetrahydrofuran- | | |
| | 2-carboxamide | | |
| 279 | (2R,3S,4S,5R)-3-(3,4-difluoro- | ESI-MS m/z calc. | |
| | 2-methoxyphenyl)-4,5- | 568.536 found 569.38 | |
| | dimethyl-N-(2-((1R,4R)-5- | (M + 1)⁺; 567.08 (M − 1)⁻; | |
| | methyl-2,5- | Retention time: 2.80 | |
| | diazabicyclo[2.2.1]heptane-2- | minutes | |
| | carbonyl)pyridin-4-yl)-5- | | |
| | (trifluoromethyl)tetrahydrofuran- | | |
| | 2-carboxamide | | |
| 280 | (2R,3S,4S,5R)-3-(3,4-difluoro- | ESI-MS m/z calc. | |
| | 2-methoxyphenyl)-4,5- | 584.578 found 585.40 | |
| | dimethyl-5-(trifluoromethyl)-N- | (M + 1)⁺; Retention time: | |
| | (2-(3,3,4-trimethylpiperazine-1- | 2.82 minutes | |
| | carbonyl)pyridin-4- | | |
| | yl)tetrahydrofuran-2- | | |
| | carboxamide | | |
| 281 | 4-((2R,3S,4S,5R)-3-(3,4- | ESI-MS m/z calc. | |
| | difluoro-2-methoxyphenyl)-4,5- | 586.551 found 587.38 | |
| | dimethyl-5- | (M + 1)⁺; 585.58 (M − 1)⁻; | |
| | (trifluoromethyl)tetrahydrofuran- | Retention time: 2.91 | |
| | 2-carboxamido)-N-((3S,4S)- | minutes | |
| | 4-methoxy-1-methylpyrrolidin- | | |
| | 3-yl)picolinamide | | |
| 282 | (2R,3S,4S,5R)-3-(3,4-difluoro- | ESI-MS m/z calc. | |
| | 2-methoxyphenyl)-4,5- | 598.562 found 599.39 | |
| | dimethyl-N-(2-((4aR,7aS)-6- | (M + 1)⁺; 597.54 (M − 1)⁻; | |
| | methyloctahydropyrrolo[3,4- | Retention time: 2.79 | |
| | b][1,4]oxazine-4- | minutes | |
| | carbonyl)pyridin-4-yl)-5- | | |
| | (trifluoromethyl)tetrahydrofuran- | | |
| | 2-carboxamide | | |
| 283 | (2R,3S,4S,5R)-3-(3,4-difluoro- | ESI-MS m/z calc. | |
| | 2-methoxyphenyl)-4,5- | 596.589 found 597.44 | |
| | dimethyl-N-(2-((1S,5S)-6- | (M + 1)⁺; 595.15 (M − 1)⁻; | |
| | methyl-3,6- | Retention time: 2.79 | |
| | diazabicyclo[3.2.2]nonane-3- | minutes | |
| | carbonyl)pyridin-4-yl)-5- | | |
| | (trifluoromethyl)tetrahydrofuran- | | |
| | 2-carboxamide | | |
| 284 | (2R,3S,4S,5R)-3-(3,4-difluoro- | ESI-MS m/z calc. | ¹H NMR (500 MHz, |
| | 2-methoxyphenyl)-4,5- | 598.562 found 599.39 | DMSO-d₆) δ 10.68 (s, |
| | dimethyl-N-(2-(9-methyl-3- | (M + 1)⁺; Retention time: | 1H), 8.43 (d, J = 5.6 Hz, |
| | oxa-7,9- | 2.74 minutes | 1H), 7.75-7.59 (m, 2H), |
| | diazabicyclo[3.3.1]nonane-7- | | 7.16 (dd, J = 16.0, 6.9 Hz, |
| | carbonyl)pyridin-4-yl)-5- | | 2H), 5.08 (d, J = 10.2 Hz, |
| | (trifluoromethyl)tetrahydrofuran- | | 1H), 4.39 (d, J = 13.4 Hz, |
| | 2-carboxamide | | 1H), 4.25 (t, J = 9.0 Hz, |
| | | | 1H), 3.95 (d, J = 2.1 Hz, |
| | | | 3H), 3.85 (d, J = 11.0 Hz, |
| | | | 1H), 3.76 (d, J = 11.2 Hz, |
| | | | 1H), 3.72 (d, J = 10.8 Hz, |
| | | | 1H), 3.64 (d, J = 13.6 Hz, |
| | | | 1H), 3.52 (d, J = 10.9 Hz, |
| | | | 1H), 3.45 (d, J = 15.9 Hz, |
| | | | 1H), 3.28 (s, 3H), 2.81- |
| | | | 2.72 (m, 1H), 2.66-2.59 |
| | | | (m, 1H), 2.43-2.33 (m, |
| | | | 2H), 1.60 (s, 3H), 0.73 (d, |
| | | | J = 7.4 Hz, 3H) ppm. |
| 285 | 4-((2R,3S,4S,5R)-3-(3,4- | ESI-MS m/z calc. | |
| | difluoro-2-methoxyphenyl)-4,5- | 600.577 found 601.44 | |
| | dimethyl-5- | (M + 1)⁺; 599.79 (M − 1)⁻; | |
| | (trifluoromethyl)tetrahydrofuran- | Retention time: 2.98 | |
| | 2-carboxamido)-N-((3S,4R)- | minutes | |
| | 4-methoxy-1-methylpiperidin- | | |
| | 3-yl)picolinamide | | |
| 286 | 4-((2R,3S,4S,5R)-3-(3,4- | ESI-MS m/z calc. | |
| | difluoro-2-methoxyphenyl)-4,5- | 600.577 found 601.29 | |
| | dimethyl-5- | (M + 1)⁺; 599.14 (M − 1)⁻; | |
| | (trifluoromethyl)tetrahydrofuran- | Retention time: 2.81 | |
| | 2-carboxamido)-N-((3S,4S)- | minutes | |
| | 4-methoxy-1-methylpyrrolidin- | | |
| | 3-yl)-N-methylpicolinamide | | |
| 287 | (2R,3S,4S,5R)-3-(3,4-difluoro- | ESI-MS m/z calc. | |
| | 2-methoxyphenyl)-N-(2-((R)-3- | 600.577 found 601.44 | |
| | (methoxymethyl)-4- | (M + 1)⁺; 599.49 (M − 1)⁻; | |
| | methylpiperazine-1- | Retention time: 2.80 | |
| | carbonyl)pyridin-4-yl)-4,5- | minutes | |
| | dimethyl-5- | | |
| | (trifluoromethyl)tetrahydrofuran- | | |
| | 2-carboxamide | | |
| 288 | (2R,3S,4S,5R)-3-(3,4-difluoro- | ESI-MS m/z calc. | ¹H NMR (500 MHz, |
| | 2-methoxyphenyl)-N-(2-((R)- | 570.551 found 571.38 | DMSO-d₆) δ 10.66 (s, |
| | 3,4-dimethylpiperazine-1- | (M + 1)⁺; 569.23 (M − 1)⁻; | 1H), 8.43 (s, 1H), 7.80 (s, |
| | carbonyl)pyridin-4-yl)-4,5- | Retention time: 2.77 | 1H), 7.64 (s, 1H), 7.21- |
| | dimethyl-5- | minutes | 7.11 (m, 2H), 5.09 (d, J = |
| | (trifluoromethyl)tetrahydrofuran- | | 10.3 Hz, 1H), 4.30-4.15 |
| | 2-carboxamide | | (m, 2H), 3.95 (d, J = 2.0 |
| | | | Hz, 3H), 3.55 (dd, J = |
| | | | 36.6, 13.0 Hz, 1H), 3.15- |
| | | | 2.96 (m, 1H), 2.85-2.73 |
| | | | (m, 2H), 2.68-2.63 (m, |
| | | | 1H), 2.17 (s, 3H), 2.11- |
| | | | 1.90 (m, 2H), 1.60 (s, |
| | | | 3H), 0.94 (dd, J = 98.3, |
| | | | 6.2 Hz, 3H), 0.77-0.69 |
| | | | (m, 3H) ppm. |
| 289 | (2R,3S,4S,5R)-3-(3,4-difluoro- | ESI-MS m/z calc. | ¹H NMR (500 MHz, |
| | 2-methoxyphenyl)-4,5- | 584.578 found 585.38 | DMSO-d₆) δ 10.65 (s, |
| | dimethyl-5-(trifluoromethyl)-N- | (M + 1)⁺; 583.33 (M − 1)⁻; | 1H), 8.41 (s, 1H), 7.76 (s, |
| | (2-((2R,5R)-2,4,5- | Retention time: 2.81 | 1H), 7.61 (s, 1H), 7.21- |
| | trimethylpiperazine-1- | minutes | 7.11 (m, 2H), 5.08 (d, J = |
| | carbonyl)pyridin-4- | | 10.0 Hz, 1H), 4.28-4.14 |
| | yl)tetrahydrofuran-2- | | (m, 2H), 3.95 (d, J = 1.9 |
| | carboxamide | | Hz, 3H), 3.85 (s, 1H), |
| | | | 2.80-2.72 (m, 1H), 2.71- |
| | | | 2.64 (m, 1H), 2.21- |
| | | | 2.16 (m, 1H), 2.16-2.10 |
| | | | (m, 4H), 1.84 (s, 1H), |
| | | | 1.60 (s, 3H), 1.24 (dd, J = |
| | | | 10.1, 6.7 Hz, 3H), 0.96 |
| | | | (dd, J = 103.7, 6.1 Hz, |
| | | | 3H), 0.72 (d, J = 7.4 Hz, |
| | | | 3H) ppm. |
| 290 | 4-((2R,3S,4S,5R)-3-(3,4- | ESI-MS m/z calc. | |
| | difluoro-2-methoxyphenyl)-4,5- | 584.578 found 585.43 | |
| | dimethyl-5- | (M + 1)⁺; 583.08 (M − 1)⁻; | |
| | (trifluoromethyl)tetrahydrofuran- | Retention time: 2.95 | |
| | 2-carboxamido)-N-((3S,5R)- | minutes | |
| | 1,5-dimethylpiperidin-3- | | |
| | yl)picolinamide | | |
| 291 | 4-((2R,3S,4S,5R)-3-(3,4- | ESI-MS m/z calc. | |
| | difluoro-2-methoxyphenyl)-4,5- | 600.577 found 601.44 | |
| | dimethyl-5- | (M + 1)⁺; 600.84 (M − 1)⁻; | |
| | (trifluoromethyl)tetrahydrofuran- | Retention time: 2.99 | |
| | 2-carboxamido)-N-((3S,4S)- | minutes | |
| | 4-ethoxy-1-methylpyrrolidin-3- | | |
| | yl)picolinamide | | |
| 292 | (2R,3S,4S,5R)-3-(3,4-difluoro- | ESI-MS m/z calc. | ¹H NMR (500 MHz, |
| | 2-methoxyphenyl)-N-(2- | 574.515 found 575.4 | DMSO-d₆) δ 10.68 (s, |
| | ((3R,4S)-3-fluoro-4- | (M + 1)⁺; 573.4 (M − 1)⁻; | 1H), 8.48 (dd, J = 7.1, 5.6 |
| | (methylamino)pyrrolidine-1- | Retention time: 3.18 | Hz, 1H), 8.02 (d, J = 2.1 |
| | carbonyl)pyridin-4-yl)-4,5- | minutes | Hz, 1H), 7.76-7.69 (m, |
| | dimethyl-5- | | 1H), 7.21-7.10 (m, 2H), |
| | (trifluoromethyl)tetrahydrofuran- | | 5.31-5.05 (m, 2H), 4.25 |
| | 2-carboxamide | | (dd, J = 10.2, 7.7 Hz, |
| | | | 1H), 4.00-3.65 (m, 6H), |
| | | | 3.38 (t, J = 10.6 Hz, 1H), |
| | | | 3.14 (t, J = 11.0 Hz, 1H), |
| | | | 2.77 (p, J = 7.5 Hz, 1H), |
| | | | 2.34 (d, J = 27.6 Hz, 3H), |
| | | | 1.60 (s, 3H), 0.73 (d, J = |
| | | | 6.9 Hz, 3H) ppm; amine |
| | | | NH not observed. |
| 293 | (2R,3S,4S,5R)-3-(3,4-difluoro- | ESI-MS m/z calc. | ¹H NMR (500 MHz, |
| | 2-methoxyphenyl)-N-(2- | 600.2371 found 601.5 | DMSO-d₆) δ 10.67 (s, |
| | ((3S,4S)-3-(dimethylamino)-4- | (M + 1)⁺; 599.4 (M − 1)⁻; | 1H), 8.48 (d, J = 5.6 Hz, |
| | methoxypyrrolidine-1- | Retention time: 3.3 | 1H), 7.98 (dd, J = 5.8, 2.1 |
| | carbonyl)pyridin-4-yl)-4,5- | minutes | Hz, 1H), 7.75-7.69 (m, |
| | dimethyl-5- | | 1H), 7.22-7.09 (m, 2H), |
| | (trifluoromethyl)tetrahydrofuran- | | 5.10 (d, J = 10.2 Hz, 1H), |
| | 2-carboxamide | | 4.25 (dd, J = 10.2, 7.7 Hz, |
| | | | 1H), 3.99-3.86 (m, 4H), |
| | | | 3.80-3.43 (m, 4H), 3.24 |
| | | | (d, J = 34.5 Hz, 3H), 2.82- |
| | | | 2.72 (m, 2H), 2.18 (d, J = |
| | | | 36.9 Hz, 6H), 1.60 (s, |
| | | | 3H), 0.73 (d, J = 7.4 Hz, |
| | | | 3H) ppm. |
| 294 | rel-(2R*,3S*,4S*,5R*)-3-(3,4- | ESI-MS m/z calc. | |
| | difluoro-2-methoxyphenyl)-4,5- | 568.536 found 569.7 | |
| | dimethyl-N-(2-((1R,6S)-5- | (M + 1)⁺; 567.6 (M − 1)⁻; | |
| | methyl-2,5- | Retention time: 3.27 | |
| | diazabicyclo[4.1.0]heptane-2- | minutes | |
| | carbonyl)pyridin-4-yl)-5- | | |
| | (trifluoromethyl)tetrahydrofuran- | | |
| | 2-carboxamide | | |
| | (Precursor was the first eluting | | |
| | isomer by SFC on a Chiralpak | | |
| | IB column, rt = 1.04 min) | | |
| 295 | rel-(2R*,3S*,4S*,5R*)-3-(3,4- | ESI-MS m/z calc. | |
| | difluoro-2-methoxyphenyl)-4,5- | 568.536 found 569.7 | |
| | dimethyl-N-(2-((1S,6R)-5- | (M + 1)⁺; 567.6 (M − 1)⁻; | |
| | methyl-2,5- | Retention time: 3.27 | |
| | diazabicyclo[4.1.0]heptane-2- | minutes | |
| | carbonyl)pyridin-4-yl)-5- | | |
| | (trifluoromethyl)tetrahydrofuran- | | |
| | 2-carboxamide | | |
| | (Precursor was the second | | |
| | eluting isomer by SFC on a | | |
| | Chiralpak IB column, rt = 1.43 | | |
| | min) | | |

The following compounds were made using methods similar to those described in Example 29, except that different amine coupling partners were used in step 3. In step 5, the conditions were pushed to form a mixture of mono methylation and dimethylation by using an excess of both formaldehyde and sodium triacetoxyborohydride:

| Cmpd No. | Compound Name | LC/MS | NMR (shifts in ppm) |
|---|---|---|---|
| 296 | (2R,3S,4S,5R)-3-(3,4-difluoro- | ESI-MS m/z calc. | ¹H NMR (500 MHz, |
| | 2-methoxyphenyl)-N-(2- | 588.2171 found 589.5 | DMSO-d₆) δ 10.70 (s, |
| | ((3S,4R)-3-(dimethylamino)-4- | (M + 1)⁺; 587.5 (M − 1)⁻; | 1H), 8.54-8.43 (m, 1H), |
| | fluoropyrrolidine-1- | Retention time: 3.25 | 8.03 (d, J = 11.2 Hz, 1H), |
| | carbonyl)pyridin-4-yl)-4,5- | minutes | 7.79-7.67 (m, 1H), 7.24- |
| | dimethyl-5- | | 7.05 (m, 2H), 6.58 (s, |
| | (trifluoromethyl)tetrahydrofuran- | | 1H), 5.38-5.00 (m, 2H), |
| | 2-carboxamide | | 4.25 (t, J = 9.2 Hz, 1H), |
| | | | 4.06-3.50 (m, 7H), 2.83- |
| | | | 2.75 (m, 1H), 2.25 (s, |
| | | | 3H), 2.18 (s, 3H), 1.60 (s, |
| | | | 3H), 0.73 (d, J = 7.3 Hz, |
| | | | 3H) ppm. |
| 297 | (2R,3S,4S,5R)-3-(3,4-difluoro- | ESI-MS m/z calc. | ¹H NMR (500 MHz, |
| | 2-methoxyphenyl)-N-(2- | 586.22144 found 587.5 | DMSO-d₆) δ 10.67 (t, J = |
| | ((3R,4S)-3-methoxy-4- | (M + 1)⁺; 585.5 (M − 1)⁻; | 2.6 Hz, 1H), 8.47 (dd, J = |
| | (methylamino)pyrrolidine-1- | Retention time: 3.2 | 5.6, 3.4 Hz, 1H), 8.00 (s, |
| | carbonyl)pyridin-4-yl)-4,5- | minutes | 1H), 7.76-7.68 (m, 1H), |
| | dimethyl-5- | | 7.23-7.10 (m, 2H), 5.10 |
| | (trifluoromethyl)tetrahydrofuran- | | (d, J = 10.2 Hz, 1H), 4.25 |
| | 2-carboxamide | | (dd, J = 10.2, 7.7 Hz, |
| | | | 1H), 3.97-3.64 (m, 7H), |
| | | | 3.52-3.37 (m, 1H), 3.36- |
| | | | 3.33 (m, 2H), 3.27- |
| | | | 3.20 (m, 3H), 2.77 (p, J = |
| | | | 7.4 Hz, 1H), 2.37-2.27 |
| | | | (m, 3H), 1.60 (s, 3H), |
| | | | 0.73 (d, J = 7.2 Hz, 3H) |
| | | | ppm. |
| 298 | (2R,3S,4S,5R)-3-(3,4-difluoro- | ESI-MS m/z calc. | ¹H NMR (500 MHz, |
| | 2-methoxyphenyl)-N-(2- | 600.2371 found 601.5 | DMSO-d₆) δ 10.68 (d, J = |
| | ((3S,4R)-3-(dimethylamino)-4- | (M + 1)⁺; 599.4 (M − 1)⁻; | 5.6 Hz, 1H), 8.48 (d, J = |
| | methoxypyrrolidine-1- | Retention time: 3.25 | 5.5 Hz, 1H), 8.01 (s, 1H), |
| | carbonyl)pyridin-4-yl)-4,5- | minutes | 7.73 (ddd, J = 12.7, 5.6, |
| | dimethyl-5- | | 1.8 Hz, 1H), 7.21-7.11 |
| | (trifluoromethyl)tetrahydrofuran- | | (m, 2H), 5.10 (d, J = 10.3 |
| | 2-carboxamide | | Hz, 1H), 4.28-4.21 (m, |
| | | | 1H), 3.95 (d, J = 1.9 Hz, |
| | | | 3H), 3.93-3.64 (m, 4H), |
| | | | 3.56-3.41 (m, 1H), 3.24 |
| | | | (d, J = 38.2 Hz, 4H), 2.77 |
| | | | (p, J = 7.5 Hz, 1H), 2.36- |
| | | | 1.98 (m, 6H), 1.60 (s, |
| | | | 3H), 0.73 (d, J = 6.9 Hz, |
| | | | 3H) ppm. |
| 299 | (2R,3S,4S,5R)-3-(3,4-difluoro- | ESI-MS m/z calc. | ¹H NMR (500 MHz, |
| | 2-methoxyphenyl)-N-(2- | 588.542 found 589.43 | DMSO-d₆) δ 10.69 (s, |
| | ((3R,4R)-3-(dimethylamino)-4- | (M + 1)⁺; 587.43 (M − 1)⁻; | 1H), 8.49 (t, J = 5.4 Hz, |
| | fluoropyrrolidine-1- | Retention time: 3.39 | 1H), 8.08-8.00 (m, 1H), |
| | carbonyl)pyridin-4-yl)-4,5- | minutes | 7.74 (dd, J = 5.5, 2.3 Hz, |
| | dimethyl-5- | | 1H), 7.23-7.11 (m, 2H), |
| | (trifluoromethyl)tetrahydrofuran- | | 5.27 (dd, J = 52.1, 27.3 |
| | 2-carboxamide | | Hz, 1H), 5.10 (d, J = 10.2 |
| | | | Hz, 1H), 4.29-4.19 (m, |
| | | | 1H), 4.11-3.55 (m, 7H), |
| | | | 3.01-2.88 (m, 1H), 2.82- |
| | | | 2.73 (m, 1H), 2.23 (s, |
| | | | 3H), 2.16 (s, 3H), 1.60 (s, |
| | | | 3H), 0.73 (d, J = 7.1 Hz, |
| | | | 3H) ppm. |
| 300 | (2R,3S,4S,5R)-3-(3,4-difluoro- | ESI-MS m/z calc. | ¹H NMR (500 MHz, |
| | 2-methoxyphenyl)-N-(2- | 588.542 found 589.43 | DMSO-d₆) δ 10.69 (s, |
| | ((3R,4S)-3-(dimethylamino)-4- | (M + 1)⁺; 587.43 (M − 1)⁻; | 1H), 8.49 (dd, J = 11.7, |
| | fluoropyrrolidine-1- | Retention time: 3.25 | 5.6 Hz, 1H), 8.04 (dd, J = |
| | carbonyl)pyridin-4-yl)-4,5- | minutes | 8.3, 2.1 Hz, 1H), 7.74 |
| | dimethyl-5- | | (ddd, J = 7.7, 5.6, 2.1 Hz, |
| | (trifluoromethyl)tetrahydrofuran- | | 1H), 7.22-7.11 (m, 2H), |
| | 2-carboxamide | | 5.23 (dd, J = 53.9, 29.8 |
| | | | Hz, 1H), 5.10 (d, J = 10.2 |
| | | | Hz, 1H), 4.25 (dd, J = |
| | | | 10.2, 7.8 Hz, 1H), 4.04- |
| | | | 3.53 (m, 7H), 3.30-3.25 |
| | | | (m, 4H), 2.85-2.67 (m, |
| | | | 1H), 2.25 (s, 3H), 2.18 (s, |
| | | | 3H), 1.60 (s, 3H) ppm. |
| 301 | (2R,3S,4S,5R)-3-(3,4-difluoro- | ESI-MS m/z calc. | ¹H NMR (500 MHz, |
| | 2-methoxyphenyl)-N-(2- | 588.542 found 589.43 | DMSO-d₆) δ 10.68 (s, |
| | ((3S,4S)-3-(dimethylamino)-4- | (M + 1)⁺; 587.38 (M − 1)⁻; | 1H), 8.49 (t, J = 5.4 Hz, |
| | fluoropyrrolidine-1- | Retention time: 3.39 | 1H), 8.07-7.99 (m, 1H), |
| | carbonyl)pyridin-4-yl)-4,5- | minutes | 7.74 (dd, J = 5.5, 2.3 Hz, |
| | dimethyl-5- | | 1H), 7.23-7.11 (m, 2H), |
| | (trifluoromethyl)tetrahydrofuran- | | 5.38-5.17 (m, 1H), 5.10 |
| | 2-carboxamide | | (d, J = 10.1 Hz, 1H), 4.29- |
| | | | 4.21 (m, 1H), 4.12- |
| | | | 3.56 (m, 7H), 2.99-2.89 |
| | | | (m, 1H), 2.82-2.72 (m, |
| | | | 1H), 2.23 (s, 3H), 2.16 (s, |
| | | | 3H), 1.60 (s, 3H), 0.73 (d, |
| | | | J = 7.5 Hz, 3H) ppm. |
| 302 | (2R,3S,4S,5R)-3-(3,4-difluoro- | ESI-MS m/z calc. | ¹H NMR (500 MHz, |
| | 2-methoxyphenyl)-N-(2- | 600.577 found 601.44 | DMSO-d₆) δ 10.68 (d, J = |
| | ((3R,4S)-3-(dimethylamino)-4- | (M + 1)⁺; 599.44 (M − 1)⁻; | 6.2 Hz, 1H), 8.48 (d, J = |
| | methoxypyrrolidine-1- | Retention time: 3.24 | 5.5 Hz, 1H), 8.00 (s, 1H), |
| | carbonyl)pyridin-4-yl)-4,5- | minutes | 7.72 (dd, J = 12.3, 5.7 Hz, |
| | dimethyl-5- | | 1H), 7.22-7.10 (m, 2H), |
| | (trifluoromethyl)tetrahydrofuran- | | 5.10 (d, J = 10.1 Hz, 1H), |
| | 2-carboxamide | | 4.28-4.19 (m, 1H), 3.95 |
| | | | (s, 3H), 3.92-3.40 (m, |
| | | | 4H), 3.29-3.19 (m, 5H), |
| | | | 2.81-2.73 (m, 1H), 2.32- |
| | | | 2.07 (m, 6H), 1.60 (s, |
| | | | 3H), 0.73 (d, J = 7.2 Hz, |
| | | | 3H) ppm. |

### Intermediate A

### rel-(2S,3R,5S)-4-[[3-(3,4-difluoro-2-methoxy-phenyl)-5-methyl-5-(trifluoromethyl)tetrahydrofuran-2-carbonyl]amino]pyridine-2-carboxamide (Intermediate A-1) and rel-(2R,3S,5R)-4-[3-(3,4-difluoro-2-methoxy-phenyl)-5-methyl-5-(trifluoromethyl)tetrahydrofuran-2-carbonyl]amino]pyridine-2-carboxamide

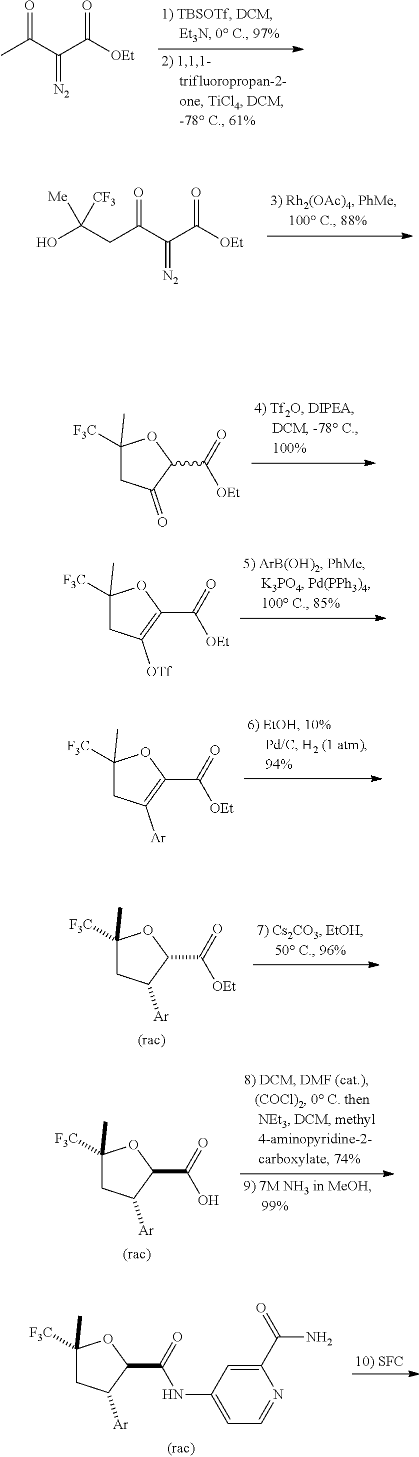

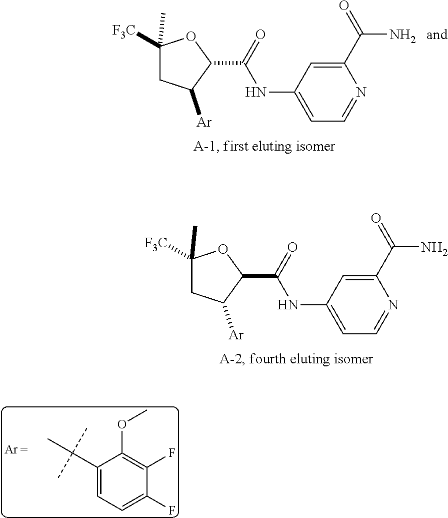

### Step 1:

To a solution of ethyl 2-diazo-3-oxo-butanoate (5.0 g, 31.4 mmol) in DCM (50 mL) stirring at 0° C. was added triethylamine (8.05 g, 11.2 mL, 78.8 mmol). TBSOTf (9.24 g, 8.2 mL, 34.3 mmol) was added slowly and the reaction mixture was stirred for 30 min at 0° C. The reaction mixture was washed with 30% NaHCO₃ solution (200 mL). The organic layer was separated and washed with water (500 mL) then dried over MgSO₄. The solvent was evaporated to give ethyl 3-[tert-butyl(dimethyl)silyl]oxy-2-diazo-but-3-enoate (8.22 g, 97%) which was used in the next step without further purification.

### Step 2:

A solution of 1,1,1-trifluoropropan-2-one (33.8 g, 27 mL, 301.2 mmol) in DCM (150 mL) was stirred at −78° C. and TiCl₄ (56.8 g, 33 mL, 299.2 mmol) was added dropwise. The reaction was kept at −78° C. for 10 min before a solution of ethyl 3-[tert-butyl(dimethyl)silyl]oxy-2-diazo-but-3-enoate (64 g, 236.7 mmol) in DCM (150 mL) was added dropwise. The reaction was kept at −78° C. for 1 h then a saturated solution of NaHCO₃ was added and the mixture diluted with DCM. The organic layer was dried over MgSO₄, concentrated in vacuo, and the residue purified by column chromatography (0 to 30% EtOAc in hexane) to give ethyl 2-diazo-6,6,6-trifluoro-5-hydroxy-5-methyl-3-oxo-hexanoate (39 g, 61%) as a pale yellow liquid. ¹H NMR (400 MHZ, Chloroform-d) δ 4.92 (s, 1H), 4.32 (q, J=7.1 Hz, 2H), 3.63 (d, J=15.5 Hz, 1H), 2.84 (d, J=15.5 Hz, 1H), 1.41 (s, 3H), 1.33 (t, J=7.1 Hz, 3H) ppm.

### Step 3:

Rhodium (II) acetate dimer (643 mg, 1.45 mmol) was charged into an oven dried two necked flask. Toluene (970 mL) was added and the solution was stirred at 100° C. for 10 min. The solution was briefly lifted out of the oil bath whilst a solution of ethyl 2-diazo-6,6,6-trifluoro-5-hydroxy-5-methyl-3-oxo-hexanoate (39 g, 145.4 mmol) in toluene (200 mL) was added dropwise, and the reaction was heated at reflux for an additional 1 h. The reaction mixture was filtered through filter paper and the filtrate was concentrated in vacuo to give ethyl 5-methyl-3-oxo-5-(trifluoromethyl)tetrahydrofuran-2-carboxylate (30.89 g, 88%). ¹H NMR (400 MHZ, Chloroform-d) δ 4.68 (s, 1H), 4.35-4.17 (m, 2H), 2.89 (d, J=18.8, 1H), 2.58 (d, J=18.8, 1H), 1.70 (s, 3H), 1.30 (t, J=7.2, Hz, 3H) ppm.

### Step 4:

Trifluoromethanesulfonic anhydride (6.0 mL, 35.7 mmol) was added dropwise to a solution of ethyl 5-methyl-3-oxo-5-(trifluoromethyl)tetrahydrofuran-2-carboxylate (6.5 g, 27.1 mmol) and DIPEA (14 mL, 80.4 mmol) in DCM (150 mL) at −78° C. and the reaction stirred for 2.5 h before saturated aqueous NH₄Cl (75 mL) was added. The mixture was warmed to ambient temperature, the layers separated, and the aqueous layer extracted with DCM (2×30 mL). The combined organic extracts were dried over MgSO₄, filtered, and concentrated in vacuo to give ethyl 2-methyl-2-(trifluoromethyl)-4-(trifluoromethylsulfonyloxy)-3H-furan-5-carboxylate (10.1 g, 100%) which was used directly in the next reaction without further purification.

### Step 5:

To a stirred solution of (3,4-difluoro-2-methoxy-phenyl) boronic acid (2.0 g, 10.6 mmol) and ethyl 2-methyl-2-(trifluoromethyl)-4-(trifluoromethylsulfonyloxy)-3H-furan-5-carboxylate (3 g, 7.90 mmol) in toluene (80 mL) was added K₃PO₄ (13 mL of 2 M aq., 26.0 mmol). The mixture was degassed with N₂ for 20 min before Pd(PPh₃)₄ (466 mg, 0.40 mmol) was added and then heated to 100° C. for 1 h. The mixture was filtered by celite pad, the filtrate diluted with water (50 mL) and the aqueous layer extracted with EtOAc (50×2 mL). The organic layer was dried over MgSO₄, filtered, and evaporated. The residue was purified by column chromatography (SiO₂, 0-2% EtOAc in hexane) to give ethyl 4-(3,4-difluoro-2-methoxy-phenyl)-2-methyl-2-(trifluoromethyl)-3H-furan-5-carboxylate (2.5 g, 85%) as a light-yellow liquid. ¹H NMR (400 MHZ, Chloroform-d) δ 6.87 (pd, J=8.8, 6.2 Hz, 2H), 4.15 (q, J=7.1 Hz, 2H), 3.89 (s, 3H), 3.42 (d, J=17.4 Hz, 1H), 2.93 (d, J=17.4 Hz, 1H), 1.65 (s, 3H), 1.14 (t, J=7.1 Hz, 3H) ppm. ESI-MS m/z calc. 366.089, found 367.2 (M+1)⁺; Retention time: 2.03 minutes.

### Step 6:

EtOH (200 mL) was added to ethyl 4-(3,4-difluoro-2-methoxy-phenyl)-2-methyl-2-(trifluoromethyl)-3H-furan-5-carboxylate (5.51 g, 15.0 mmol) and Pd/C (10 wt. % loading, 2.2 g, 2.067 mmol). The mixture was degassed and stirred under a balloon of H₂ for 96 h. The catalyst was removed by filtration, the solids washed with EtOH (50 mL) and the filtrate concentrated in vacuo. A further portion of Pd/C (10 wt. % loading, 2.2 g, 2.07 mmol) was added to the residue followed by EtOH (200 mL) and the reaction mixture stirred under a balloon of H₂ at ambient temperature for 24 h. The catalyst was removed by filtration, the solids washed with EtOH (50 mL) and the filtrate concentrated in vacuo. A further portion of Pd/C (10 wt. % loading, 2.2 g, 2.07 mmol) was added to the residue followed by EtOH (200 mL) and the reaction mixture stirred under a balloon of H₂ at ambient temperature for 4 days. The catalyst was removed by filtration, the solids washed with EtOH (50 mL) and the filtrate concentrated in vacuo to give ethyl rac-(2S,3S,5R)-3-(3,4-difluoro-2-methoxy-phenyl)-5-methyl-5-(trifluoromethyl)tetrahydrofuran-2-carboxylate (5.19 g, 94%) as a white solid, and as a single diastereomer. ¹H NMR (500 MHZ, Chloroform-d) δ 6.89-6.86 (m, 1H), 6.82-6.77 (m, 1H), 4.93 (d, J=8.9 Hz, 1H), 4.23 (dt, J=13.0, 7.6 Hz, 1H), 4.08 (d, J=2.9 Hz, 3H), 3.85-3.71 (m, 2H), 2.82 (t, J=12.5 Hz, 1H), 2.04 (dd, J=12.0, 6.7 Hz, 1H), 1.53 (s, 3H), 0.94 (t, J=7.1 Hz, 3H) ppm; ¹⁹F NMR (471 MHZ, Chloroform-d) δ −80.15, −136.84 (d, J=19.4 Hz), −154.77 (d, J=19.6 Hz) ppm.

### Step 7:

Ethyl rac-(2S,3S,5R)-3-(3,4-difluoro-2-methoxy-phenyl)-5-methyl-5-(trifluoromethyl)tetrahydrofuran-2-carboxylate (5.19 g, 14.09 mmol) was dissolved in ethanol (100 mL). Cesium carbonate (7.1 g, 21.8 mmol) was added and the suspension stirred at 50° C. for 2 h. The reaction mixture was concentrated in vacuo and the residue partitioned between 1M HCl and MTBE. The layers were separated and the aqueous layer was extracted twice with MTBE. The combined organic extracts were dried (MgSO₄), filtered and concentrated in vacuo to give rac-(2R,3S,5R)-3-(3,4-difluoro-2-methoxy-phenyl)-5-methyl-5-(trifluoromethyl)tetrahydrofuran-2-carboxylic acid (5.11 g, 96%) as a colourless oil, as a single diastereomer. ¹H NMR (500 MHZ, Chloroform-d) δ 6.99-6.96 (m, 1H), 6.92-6.87 (m, 1H), 4.68 (d, J=10.5 Hz, 1H), 4.00 (d, J=2.7 Hz, 3H), 3.90 (ddd, J=12.0, 10.6, 8.2 Hz, 1H), 2.58 (t, J=12.5 Hz, 1H), 2.31 (dd, J=13.0, 8.2 Hz, 1H), 1.60 (s, 3H) ppm; ¹⁹F NMR (471 MHZ, Chloroform-d) δ −81.56, −136.40 (d, J=19.6 Hz), −153.60 (d, J=19.5 Hz) ppm. ESI-MS m/z calc. 340.0734, found 339.5 (M−1); Retention time: 0.52 minutes.

### Step 8:

To a solution of rac-(2R,3S,5R)-3-(3,4-difluoro-2-methoxy-phenyl)-5-methyl-5-(trifluoromethyl)tetrahydrofuran-2-carboxylic acid (1.5 g, 4.41 mmol) in DCM (30 mL) cooled to −10° C. was added DMF (5 μL, 0.065 mmol) followed by oxalyl chloride (620 μL, 7.11 mmol). The reaction was stirred for 4 h, allowing it to warm to ambient temperature before further oxalyl chloride (300 μL, 3.55 mmol) was added. The reaction was stirred for a further h before being concentrated in vacuo. The residue was dissolved in DCM (30 mL) and the solution cooled in an ice bath. TEA (600 μL, 4.31 mmol) and methyl 4-aminopyridine-2-carboxylate (663.7 mg, 4.36 mmol) were sequentially added and the resultant mixture stirred for 30 min before being quenched with MeOH and concentrated in vacuo. Purification by flash chromatography (40 g SiO₂, 0 to 60% ethyl acetate in heptane, loaded in DCM) gave methyl rac-(2R,3S,5R)-4-[[3-(3,4-difluoro-2-methoxy-phenyl)-5-methyl-5-(trifluoromethyl)tetrahydrofuran-2-carbonyl]amino]pyridine-2-carboxylate (827.6 mg, 74%). ¹H NMR (500 MHZ, Chloroform-d) δ 8.63 (d, J=5.5 Hz, 1H), 8.46 (s, 1H), 8.07 (d, J=2.1 Hz, 1H), 7.94 (dd, J=5.5, 2.2 Hz, 1H), 7.00 (ddd, J=8.0, 5.5, 2.1 Hz, 1H), 6.90 (td, J=9.1, 7.3 Hz, 1H), 4.75 (d, J=10.7 Hz, 1H), 4.01 (s, 3H), 3.99 (d, J=2.6 Hz, 3H), 3.83 (td, J=11.4, 8.3 Hz, 1H), 2.61 (t, J=12.5 Hz, 1H), 2.34 (dd, J=13.1, 8.2 Hz, 1H), 1.65 (s, 3H) ppm. ESI-MS m/z calc. 474.1214, found 474.7 (M+1)+ and 473.2 (M−1); Retention time: 0.92 minutes.

### Step 9:

Methyl rac-(2R,3S,5R)-4-[[3-(3,4-difluoro-2-methoxy-phenyl)-5-methyl-5-(trifluoromethyl)tetrahydrofuran-2-carbonyl]amino]pyridine-2-carboxylate (1.9 g, 4.01 mmol) was dissolved in methanolic ammonia (20 mL of 7 M, 140.0 mmol) and the reaction stirred at ambient temperature overnight. Additional methanolic ammonia (5 mL of 7 M, 35.0 mmol) was added and reaction stirred at ambient temperature for a further 3 h before being concentrated in vacuo to give rac-4-((2R,3S,5R)-3-(3,4-difluoro-2-methoxyphenyl)-5-methyl-5-(trifluoromethyl)tetrahydrofuran-2-carboxamido) picolinamide (1.94 g, 99%). ¹H NMR (500 MHZ, Methanol-d₄) δ 8.49 (dd, J=5.5, 0.6 Hz, 1H), 8.26 (dd, J=2.2, 0.6 Hz, 1H), 7.88 (dd, J=5.5, 2.2 Hz, 1H), 7.14 (ddd, J=8.3, 5.7, 2.3 Hz, 1H), 6.99 (ddd, J=9.9, 8.9, 7.5 Hz, 1H), 4.67 (d, J=10.3 Hz, 1H), 4.10-4.01 (m, 1H), 3.92 (d, J=2.3 Hz, 3H), 3.35 (s, 3H), 2.62 (t, J=12.4 Hz, 1H), 2.40 (dd, J=12.8, 8.2 Hz, 1H), 1.63 (s, 3H) ppm. ESI-MS m/z calc. 459.12173, found 460.2 (M+1)+ and 458.3 (M−1); Retention time: 3.07 minutes.

### Step 10:

rac-4-((2R,3S,5R)-3-(3,4-difluoro-2-methoxyphenyl)-5-methyl-5-(trifluoromethyl)tetrahydrofuran-2-carboxamido) picolinamide (1.9 g, 3.89 mmol) was separated by chiral SFC using a (R,R)-Whelk-O1 column, 5 μm particle size, 25 cm×21.2 mm from Regis Technologies to give two single isomers of unknown absolute configuration:

First Eluting Isomer (rt=5.05 min): rel-4-(2S,3R,5S)-3-(3,4-difluoro-2-methoxyphenyl)-5-methyl-5-(trifluoromethyl)tetrahydrofuran-2-carboxamido) picolinamide (Intermediate A-1, 724 mg, 38%). ¹H NMR (500 MHZ, Methanol-d₄) δ 8.36 (d, J=5.5 Hz, 1H), 8.13 (d, J=2.1 Hz, 1H), 7.75 (dd, J=5.5, 2.2 Hz, 1H), 7.00 (ddd, J=8.2, 5.6, 2.2 Hz, 1H), 6.86 (td, J=9.3, 7.5 Hz, 1H), 4.55 (d, J=10.3 Hz, 1H), 3.92 (ddd, J=12.2, 10.4, 8.2 Hz, 1H), 3.79 (d, J=2.3 Hz, 3H), 3.22 (s, 1H), 2.49 (t, J=12.4 Hz, 1H), 2.27 (dd, J=12.8, 8.2 Hz, 1H), 1.50 (s, 3H) ppm. ESI-MS m/z calc. 459.12173, found 460.2 (M+1)+ and 458.3 (M−1); Retention time: 3.06 minutes.

Second Eluting Isomer (rt=7.36 min): rel-4-((2R,3S,5R)-3-(3,4-difluoro-2-methoxyphenyl)-5-methyl-5-(trifluoromethyl)tetrahydrofuran-2-carboxamido) picolinamide (Intermediate A-2, 749 mg, 39%). ¹H NMR (500 MHZ, Methanol-d₄) δ 8.36 (d, J=5.5 Hz, 1H), 8.13 (d, J=2.2 Hz, 1H), 7.75 (dd, J=5.5, 2.2 Hz, 1H), 7.01 (ddd, J=8.3, 5.6, 2.2 Hz, 1H), 6.86 (td, J=9.4, 7.5 Hz, 1H), 4.55 (d, J=10.2 Hz, 1H), 3.92 (ddd, J=12.0, 10.4, 8.2 Hz, 1H), 3.79 (d, J=2.3 Hz, 3H), 3.22 (s, 3H), 2.49 (t, J=12.4 Hz, 1H), 2.27 (dd, J=12.9, 8.2 Hz, 1H), 1.50 (s, 3H) ppm. ESI-MS m/z calc. 459.12173, found 460.2 (M+1)+ and 458.3 (M−1); Retention time: 3.06 minutes.

### Intermediate B

### 2-(4-(benzyloxy)-3-fluoro-2-methoxyphenyl) acetic acid

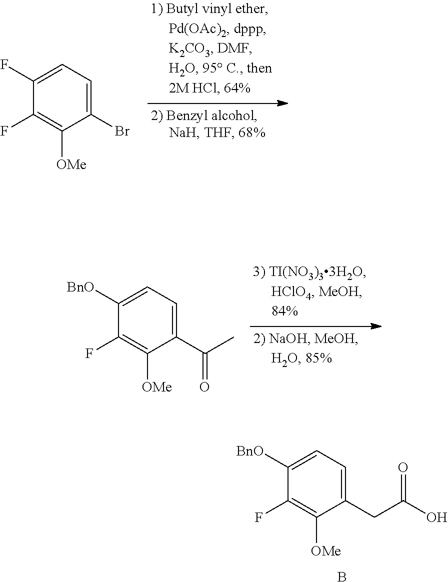

### Step 1:

A mixture of 1-bromo-3,4-difluoro-2-methoxybenzene (5 g, 22.42 mmol), butyl vinyl ether (9 mL, 66.49 mmol), K₂CO₃ (3.7372 g, 27.04 mmol), dppp (612.81 mg, 1.486 mmol), and Pd(OAc)₂ (151.96 mg, 0.677 mmol) in DMF (50 mL) and H₂O (5 mL) was degassed (vacuum nitrogen cycles×3) and heated to 95° C. overnight. The reaction mixture was cooled down to ambient temperature. 2 M HCl (80 mL, 160.0 mmol) was added and the mixture was stirred at ambient temperature for 30 min. The mixture was extracted with EtOAc (2×). The combined organic phases were washed with a saturated NaHCO₃ solution and brine, dried over MgSO₄, filtered, and concentrated in vacuo. Purification by flash chromatography (120 g SiO₂, 0 to 5% EtOAc in hexanes) gave 1-(3,4-difluoro-2-methoxyphenyl) ethan-1-one (2.687 g, 64%) as a yellow oil. ¹H NMR (400 MHZ, Chloroform-d) δ 7.50 (ddd, J=9.0, 6.1, 2.3 Hz, 1H), 6.92 (td, J=9.0, 6.9 Hz, 1H), 4.08 (d, J=2.7 Hz, 3H), 2.60 (s, 3H) ppm. ¹⁹F NMR (376 MHZ, Chloroform-d) δ −129.21 (d, J=19.0 Hz), −153.39 (d, J=19.0 Hz) ppm.

### Step 2:

A solution of benzyl alcohol (2.9 g, 26.818 mmol) in DMF (10 mL) was added to a stirred suspension of sodium hydride (1.05 g, 60% w/w, 26.253 mmol) in DMF (40 mL) at room temperature. After stirring the mixture for 5 min, 1-(3,4-difluoro-2-methoxyphenyl) ethan-1-one (5 g, 26.859 mmol) was added and the stirring was continued at room temperature for 30 min. 2 N HCl (10 mL) and brine (100 mL) were added and the mixture was extracted with ethyl acetate (100 mL then 50 mL). The combined organic extracts were washed with brine (2×20 mL), dried over Na₂SO₄, filtered and concentrated in vacuo. Purification by flash chromatography (SiO₂, 10 to 30% EtOAc in heptane) gave 1-(4-(benzyloxy)-3-fluoro-2-methoxyphenyl) ethan-1-one (5.03 g, 68%) as a yellow solid. ¹H NMR (400 MHz, Chloroform-d) δ 7.51 (dd, J=8.9, 2.1 Hz, 1H), 7.44-7.34 (m, 5H), 6.76 (dd, J=8.9, 7.1 Hz, 1H), 5.17 (s, 2H), 4.03 (d, J=2.3 Hz, 3H), 2.58 (s, 3H) ppm. ESI-MS m/z calc. 274.1005, found 273.02 (M−1); Retention time: 0.98 minutes.

### Step 3:

A solution of 1-(4-(benzyloxy)-3-fluoro-2-methoxyphenyl) ethan-1-one (14.8 g, 53.958 mmol) in methanol (50 mL) was added dropwise to a stirred solution of TI(NO₃)₃·3H₂O (24 g, 54.0 mmol) and perchloric acid (50 mL of 60% w/v in water, 298.63 mmol) in methanol (200 mL). The mixture was stirred at room temperature for 4.5 h. The precipitate was filtered and washed with methanol (2×50 mL). The methanolic filtrate was poured into water (1 L) and extracted with dichloromethane (2×200 mL). The combined organic extracts were washed with water (100 mL) and brine (50 mL), dried over Na₂SO₄, filtered and concentrated in vacuo to give methyl 2-(4-benzyloxy-3-fluoro-2-methoxy-phenyl)acetate (15.25 g, 84%) as a yellow oil, which was used as is in the next step. ¹H NMR (400 MHZ, Chloroform-d) & 7.44-7.32 (m, 5H), 6.84 (dd, J=8.7, 1.8 Hz, 1H), 6.67 (t, J=8.2 Hz, 1H), 5.11 (s, 2H), 3.93 (d, J=1.8 Hz, 3H), 3.69 (s, 3H), 3.58 (s, 2H) ppm. ESI-MS m/z calc. 304.1111, found 305.19 (M+1)⁺; Retention time: 2.44 minutes.

### Step 4:

Methyl 2-(4-benzyloxy-3-fluoro-2-methoxy-phenyl)acetate (15.2 g, 49.949 mmol) was added to a solution of sodium hydroxide (6 g, 150.01 mmol) in methanol (30 mL) and water (10 mL). The solution was stood at room temperature for 14 h giving an orange solid. The crude product was diluted with 2 N sodium hydroxide solution (200 mL) and extracted with dichloromethane (2×30 mL). The aqueous layer was acidified with 6 M hydrochloric acid (100 mL) and extracted with dichloromethane-isopropanol (9:1, 2×150 mL). The combined organic extracts were dried over Na₂SO₄, filtered, and concentrated in vacuo to give 2-(4-(benzyloxy)-3-fluoro-2-methoxyphenyl) acetic acid (13.15 g, 85%) as an orange solid. ¹H NMR (400 MHZ, Chloroform-d) δ 7.46-7.33 (m, 5H), 6.85 (dd, J=8.7, 1.8 Hz, 1H), 6.69 (t, J=8.0 Hz, 1H), 5.11 (s, 2H), 3.96 (d, J=2.3 Hz, 3H), 3.62 (s, 2H) ppm; OH acid not observed. ESI-MS m/z calc. 290.0954, found 289.0 (M−1); Retention time: 2.19 minutes.

### Intermediate C

### (R)-4,4,4-trifluoro-3-hydroxy-3-methylbutan-2-one

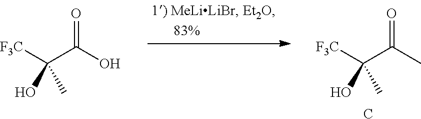

### Step 1:

A jacketed glass reactor, dried and placed under nitrogen atmosphere, was charged with (R)-3,3,3-trifluoro-2-hydroxy-2-methylpropanoic acid (1.0 kg, 6.3261 mol) and diethyl ether (10 L). Methyllithium lithium bromide complex (3.4 L of 1.5 M in Et₂O, 5.1000 mol) was added slowly with evolution of gas and heat formation. The reactor was cooled to maintain a temperature of approximately 16° C. Then methyllithium with lithium bromide (6.1 L of 2.2 M in Et₂O, 13.420 mol) was added slowly. After addition of a total of 2 equivalents, the evolution of gas stopped and the rate of addition was decreased. The mixture was stirred overnight at ambient temperature. The reaction mixture was cooled to 0° C. and transferred to an extraction flask carrying a mixture of water (6 L), ice (2 L) and brine (2 L). The mixture was neutralized by the addition of citric acid (1.6 kg, 960.96 mL, 8.3280 mol) and was stirred for 30 min. The aqueous phase was separated and extracted with diethyl ether (2×2.5 L). The combined organic layers were concentrated in vacuo to approximately 2 L. The distillate was colored yellow and consisted of 0.8% w/w product. After further distillation only 25 g of product was recovered from the distillate. The distillation residue was further concentrated in a distillation setup with vigreux (30 cm height) at normal pressure. The distillation was continued at reduced pressure (770 mbar) and the pressure was gradually lowered (until 200 mbar) with the collection flask cooled in ice and a cold trap between pump and setup. Mixed fractions were collected until the distillation temperature reached 71° C. The major fraction (590 g) was then collected until the distillation temperature dropped below 70° C. The combined mixed fractions were poured in brine and extracted with diethyl ether (3×75 mL). The combined organic layers were dried over Na₂SO₄, filtered, and concentrated in a distillation setup at normal pressure. The product was distilled at reduced pressure (200 mbar) to give the product as a colourless oil (198 g). The collected mixed fractions were redistilled to afford more product (44.25 g). All portions of product were combined (857 g), dried by addition of potassium carbonate (52 g) and left standing for 6 h. The water level dropped below detectable level and the mixture was filtered over a glass filter to give (R)-4,4,4-trifluoro-3-hydroxy-3-methylbutan-2-one (815 g, 83%) as a colourless oil (815 g).

¹H NMR (300 MHz, Chloroform-d) δ 4.33 (s, 1H), 2.40 (d, J=1.1 Hz, 3H), 1.57 (d, J=1.1 Hz, 3H) ppm. ¹⁹F NMR (282 MHZ, Chloroform-d) δ −77.96 ppm.

### Intermediate D

### 2-(6-(difluoromethyl)-2-methoxypyridin-3-yl) acetic acid

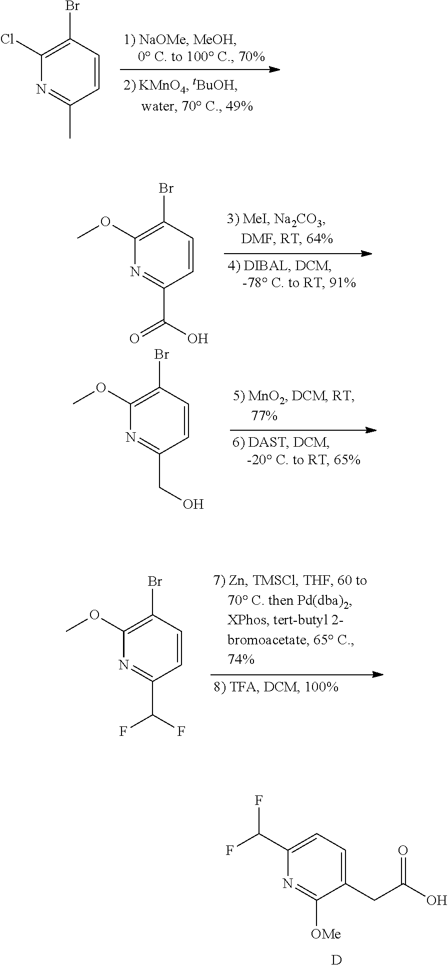

### Step 1:

Sodium methoxide (20 mL of 25% w/v solution in MeOH, 92.552 mmol) was added at 0° C. to a stirred solution of 3-bromo-2-chloro-6-methylpyridine (8 g, 38.747 mmol) in MeOH (50 mL) in a sealed tube. The reaction mixture was heated at 100° C. for 16 h. The reaction mixture was concentrated in vacuo. The residue was diluted with water (100 mL) and extracted with EtOAc (3×100 mL). The combined organic layers were dried over Na₂SO₄, filtered, and concentrated in vacuo to give 3-bromo-2-methoxy-6-methylpyridine (5.5 g, 70%) as a colourless oil. ¹H NMR (400 MHZ, DMSO-d₆) δ 7.82 (d, J=7.7 Hz, 1H), 6.76 (d, J=7.7 Hz, 1H), 3.88 (s, 3H), 2.35 (s, 3H) ppm. ESI-MS m/z calc. 200.9789, found 202.01 (M+1)⁺; Retention time: 1.69 minutes.

### Step 2:

KMnO₄ (13 g, 82.261 mmol) was added at ambient temperature to a stirred solution of 3-bromo-2-methoxy-6-methylpyridine (5.5 g, 27.221 mmol) in tert-butanol (150 mL) and water (300 mL). The reaction mixture was heated at 70° C. for 16 h. The reaction mixture was quenched by addition of a 1M aqueous solution of HCl (80 mL). The resulting mixture was stirred for 30 min, filtered and washed with EtOAc (2×100 mL). The mother liquors were extracted with EtOAc (2×50 mL). The combined organic layers were washed with a 0.5 N aqueous solution of NaOH (2×100 mL). The aqueous layer was collected, acidified by addition of a 12N aqueous solution of HCl and extracted with DCM (2×100 mL). The combined organic extracts were washed with brine, dried over Na₂SO₄, filtered, and concentrated in vacuo to give 5-bromo-6-methoxypyridine-2-carboxylic acid (3.1 g, 49%) as white solid. ¹H NMR (400 MHZ, DMSO-d₆) δ 13.27 (br s, 1H), 8.19 (d, J=7.9 Hz, 1H), 7.57 (d, J=7.9 Hz, 1H), 3.98 (s, 3H) ppm. ESI-MS m/z calc. 230.9531, found 232.0 (M+1)⁺; Retention time: 1.34 minutes.

### Step 3:

Sodium carbonate (1.5 g, 14.153 mmol) was added to a stirred solution of 5-bromo-6-methoxypyridine-2-carboxylic acid (3 g, 12.929 mmol) in DMF (40 mL). Methyl iodide (3.8760 g, 1.7 mL, 27.308 mmol) was added and the mixture was stirred at ambient temperature for 16 h. The reaction mixture was quenched by addition of ice cold water (50 mL). The aqueous phase was extracted with ethyl acetate (3×50 mL). The combined organic layers were washed with water (2×100 mL), brine (50 mL), dried over Na₂SO₄, filtered, and concentrated in vacuo to give methyl 5-bromo-6-methoxypyridine-2-carboxylate (2.02 g, 64%) as an off-white solid. ¹H NMR (400 MHZ, DMSO-d₆) δ 8.22 (d, J=7.8 Hz, 1H), 7.59 (d, J=7.8 Hz, 1H), 3.98 (s, 3H), 3.87 (s, 3H) ppm. ESI-MS m/z calc. 244.9688, found 246.1 (M+1)⁺; Retention time: 3.21 minutes.

### Step 4:

Diisobutylaluminum hydride (14 mL of 25% w/v solution in toluene, 24.610 mmol) was added at −78° C. to a stirred solution of methyl 5-bromo-6-methoxypyridine-2-carboxylate (2 g, 8.128 mmol) in DCM (80 mL). The reaction mixture was stirred at ambient temperature for 1 h. The reaction mixture was quenched by addition of a saturated aqueous solution of sodium tartrate (50 mL). The mixture was stirred for 30 min then extracted with DCM (3×100 mL). The combined organic layers were dried over Na₂SO₄, filtered, and concentrated in vacuo to give (5-bromo-6-methoxy-2-pyridyl) methanol (1.62 g, 91%) as a white solid. ¹H NMR (400 MHZ, DMSO-d₆) δ 7.99 (d, J=7.8 Hz, 1H), 7.00 (d, J=7.6 Hz, 1H), 5.45 (t, J=11.8 Hz, 1H), 4.45 (d, J=5.9 Hz, 2H), 3.89 (s, 3H) ppm. ESI-MS m/z calc. 216.9738, found 218.0 (M+1)⁺; Retention time: 2.93 minutes.

### Step 5:

MnO₂ (8 g, 92.021 mmol) was added to a stirred solution of (5-bromo-6-methoxy-2-pyridyl) methanol (1.6 g, 7.3378 mmol) in DCM (80 mL). The reaction mixture was stirred at ambient temperature for 16 h. The reaction mixture was filtered and concentrated in vacuo to give 5-bromo-6-methoxypyridine-2-carbaldehyde (1.22 g, 77%) as off-white solid. ¹H NMR (400 MHZ, DMSO-d₆) δ 9.88 (s, 1H), 8.29 (d, J=7.7 Hz, 1H), 7.49 (d, J=7.7 Hz, 1H), 4.03 (s, 3H) ppm.

### Step 6:

DAST (1.9740 g, 1.5 mL, 12.246 mmol) was slowly added at −20° C. to a stirred solution of 5-bromo-6-methoxypyridine-2-carbaldehyde (1.2 g, 5.5547 mmol) in DCM (30.000 mL). The reaction mixture was stirred at ambient temperature for 16 h. The reaction mixture was quenched by addition of ice-water. The pH of the solution was adjusted to 8-10 by addition of solid sodium hydrogen carbonate. The organic phase was collected, washed with water and brine, dried over Na₂SO₄, filtered, and concentrated in vacuo. Purification by flash chromatography (SiO₂, 100% hexanes) gave 3-bromo-6-(difluoromethyl)-2-methoxypyridine (900 mg, 65%) as a pale yellow oil. ¹H NMR (400 MHZ, DMSO-d₆) δ 8.22 (d, J=7.8 Hz, 1H), 7.23 (d, J=7.7 Hz, 1H), 7.03-6.75 (m, 1H), 3.96 (s, 3H) ppm.

### Step 7:

Chlorotrimethylsilane (513.60 mg, 0.6 mL, 4.7275 mmol) was added to a stirred solution of zinc (4.1 g, 62.701 mmol) in THF (80 mL). The reaction mixture was heated at 60° C. for 2 h. tert-Butyl 2-bromoacetate (9.6600 g, 6.9 mL, 49.525 mmol) was added dropwise and the reaction mixture was heated at 65° C. for 20 min. A degassed solution of 3-bromo-6-(difluoromethyl)-2-methoxypyridine (9.2 g, 36.718 mmol), XPhos (875 mg, 1.836 mmol) and Pd(dba)₂ (633 mg, 1.101 mmol) in THF (200 mL) was added dropwise over 15 min. The reaction mixture was heated at 65° C. for a further 2 h. After cooling to ambient temperature, a saturated aqueous ammonium chloride solution (200 mL) was added and the phases were separated. The aqueous phase was extracted with ethyl acetate (200 mL). The combined organic extracts were washed with brine (100 mL), dried over MgSO₄, filtered, and concentrated in vacuo to give tert-butyl 2-(6-(difluoromethyl)-2-methoxypyridin-3-yl)acetate (13.4 g, 74%) as a brown oil. ¹H NMR (300 MHz, Chloroform-d) δ 7.56 (d, J=7.6 Hz, 1H), 7.16 (d, J=7.6 Hz, 1H), 6.50 (t, J=55.6 Hz, 1H), 3.96 (s, 3H), 3.52 (s, 2H), 1.45 (s, 9H) ppm.

### Step 8:

Trifluoroacetic acid (8.9400 g, 6 mL, 78.405 mmol) was added to a solution of tert-butyl 2-(6-(difluoromethyl)-2-methoxypyridin-3-yl)acetate (11.2 g, 11.066 mmol) in DCM (20 mL). The reaction mixture was stirred at ambient temperature for 16 h. The reaction mixture was concentrated in vacuo. The residue was dissolved in MTBE (150 mL) and washed with a saturated aqueous sodium bicarbonate solution (3×50 mL). The combined aqueous layers were acidified with 6M HCl and extracted with MTBE (2×50 mL). The combined organic extracts were dried over MgSO₄, filtered, and concentrated in vacuo to give 2-(6-(difluoromethyl)-2-methoxypyridin-3-yl) acetic acid (2.7 g, 100%) as a pale brown solid. ¹H NMR (300 MHz, Chlororform-d) δ 7.58 (d, J=7.2 Hz, 1H), 7.18 (d, J=7.2 Hz, 1H), 6.51 (t, J=55.6 Hz, 1H), 3.98 (s, 3H), 3.66 (s, 2H) ppm; acid OH not observed.

### Intermediate E

### 2-(2-methoxy-3-(trifluoromethyl)phenyl) acetic acid

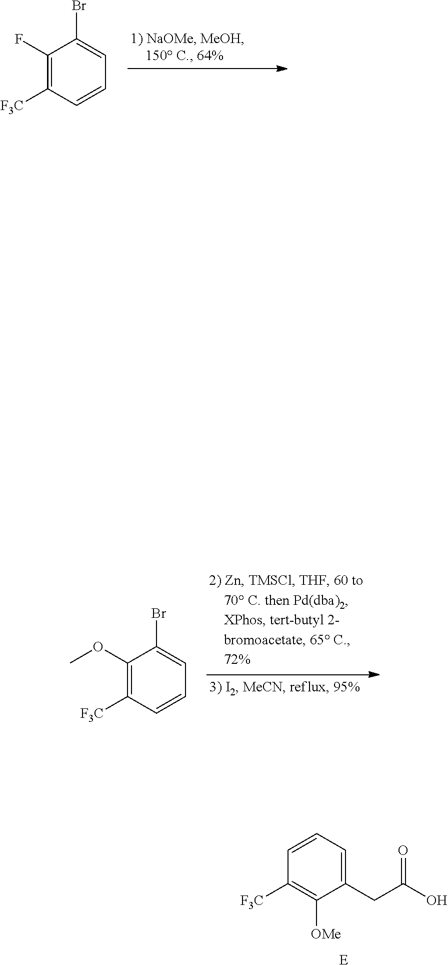

### Step 1:

Sodium methoxide (13 mL of 25% w/v, 60.16 mmol) was added to a stirred solution of 1-bromo-2-fluoro-3-(trifluoromethyl)benzene (15 g, 61.729 mmol) in MeOH (60 mL) at ambient temperature. The reaction mixture was heated at 150° C. for 3 h. The mixture was concentrated in vacuo. The residue was diluted with EtOAc (150 ml), washed with a CuSO₄ solution (80 ml), brine (80 mL), dried over Na₂SO₄, filtered, and concentrated in vacuo to give 1-bromo-2-methoxy-3-(trifluoromethyl)benzene (10 g, 64%) as an oil.

### Step 2:

A 250 mL 3 neck round bottom flask, equipped with a condenser and a magnetic stirrer was charged with THF (25 mL) and Zn (4.993 g, 0.7 mL, 76.359 mmol) under a nitrogen atmosphere. TMSCl (856 mg, 1 mL, 7.879 mmol) was added in one portion and the reaction mixture was heated to 60° C. for 45 min. A solution of tert-butyl 2-bromoacetate (15.960 g, 12 mL, 81.823 mmol) in THF (4 mL) was added dropwise over 15 min causing a 7° exotherm. The temperature was maintained at 67-68° C. for 1 h. The reaction mixture was cooled down to 40° C. and a solution of 1-bromo-2-methoxy-3-(trifluoromethyl)benzene (10 g, 39.211 mmol) in THF (4 mL) was added. The mixture was purged with nitrogen for 10 min. Pd(dba)₂ (1 g, 1.739 mmol) and XPhos (1.9 g, 3.986 mmol) were added. The reaction mixture was heated to 67° C. After 1 h, the reaction mixture was cooled down to ambient temperature and stirred overnight. The reaction mixture was cooled with an ice bath and quenched by adding a 20% aqueous ammonium chloride solution (25 mL, 2.5 vol) over 2 min. The mixture was filtered through a pad of celite. The organic layer was separated and washed with water (50 mL, 5 vol) and a 15% aqueous sodium chloride solution (50 mL, 5 vol). The aqueous layer was extracted with EtOAc (50 mL). The combined organic extracts were washed with a 15% aqueous sodium chloride solution (50 mL), dried over Na₂SO₄, filtered, and concentrated in vacuo. Purification by flash chromatography (SiO₂, 2 to 5% ethyl acetate in hexanes) gave tert-butyl 2-(2-methoxy-3-(trifluoromethyl)phenyl)acetate (8.2 g, 72%) as a yellow solid. ¹H NMR (400 MHZ, DMSO-d₆) δ 7.59 (d, J=7.76 Hz, 2H), 7.30 (t, J=7.76 Hz, 1H), 3.75 (s, 3H), 3.68 (s, 2H), 1.40 (s, 9H) ppm.

### Step 3:

I₂ (525 mg, 2.0685 mmol) was added to a stirred solution of tert-butyl 2-(2-methoxy-3-(trifluoromethyl)phenyl)acetate (3 g, 10.335 mmol) in MeCN (20 mL). The reaction mixture was stirred at reflux under an argon atmosphere for 6 h. The mixture was partitioned between EtOAc and a saturated aqueous sodium thiosulfate solution (10 mL). The aqueous phase was separated and extracted with EtOAc (50 mL). The combined organic extracts were washed with water (10 mL) and brine (10 mL), dried over Na₂SO₄, filtered, and concentrated in vacuo to give 2-(2-methoxy-3-(trifluoromethyl)phenyl) acetic acid (2.3 g, 95%) as an oil. ¹H NMR (400 MHZ, DMSO-d₆) δ 12.53 (s, 1H), 7.62-7.57 (m, 2H), 7.29 (t, J=7.84 Hz, 1H), 3.75 (s, 3H), 3.69 (s, 2H) ppm.

### Intermediate F

### 2-(3-chloro-2-methoxyphenyl) acetic acid

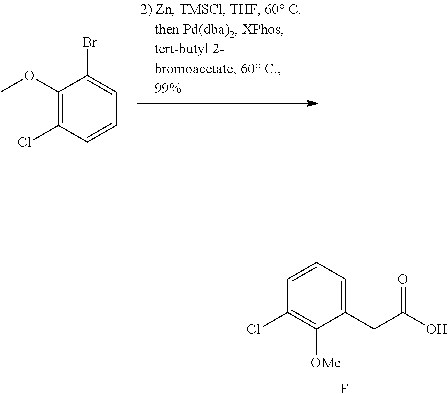

### Step 1:

Chlorotrimethylsilane (1.027 g, 1.2 mL, 9.455 mmol) was added to a slurry of zinc powder (7 g, 107.05 mmol) in anhydrous tetrahydrofuran (100 mL) and the mixture was stirred under argon at 60° C. for 2 h. tert-Butyl 2-bromoacetate (17.157 g, 12.9 mL, 87.960 mmol) was slowly added to this mixture. The mixture was stirred at 60° C. for 1 h, giving a clear solution with a small amount of unreacted zinc dust. In a second flask, a solution of 1-bromo-3-chloro-2-methoxybenzene (15 g, 67.727 mmol) and Pd₂(dba)₃ (1.55 g, 1.693 mmol) in tetrahydrofuran (150 mL) was degassed with argon for 10 min before adding the freshly prepared organozincate solution via cannula. The reaction mixture was heated at 60° C. for 2 h, then left to stir at ambient temperature for 16 h. The mixture was quenched by addition of a 10% citric acid solution (60 mL). The mixture was diluted with ethyl acetate (100 mL). The organic phase was separated, washed with brine (50 mL), dried over Na₂SO₄, filtered, and concentrated in vacuo to give 2-(3-chloro-2-methoxyphenyl) acetic acid (30 g, 99%) as a red solid. ¹H NMR (400 MHZ, Chloroform-d) δ 7.31 (dd, J=8.0, 1.6 Hz, 1H), 7.15 (dd, J=7.6, 1.6 Hz, 1H), 7.02 (t, J=7.8 Hz, 1H), 3.87 (s, 3H), 3.72 (s, 2H) ppm; acid OH not observed.

### Intermediate G

### 4-Fluoro-2-methoxy-3-methylphenyl) boronic acid

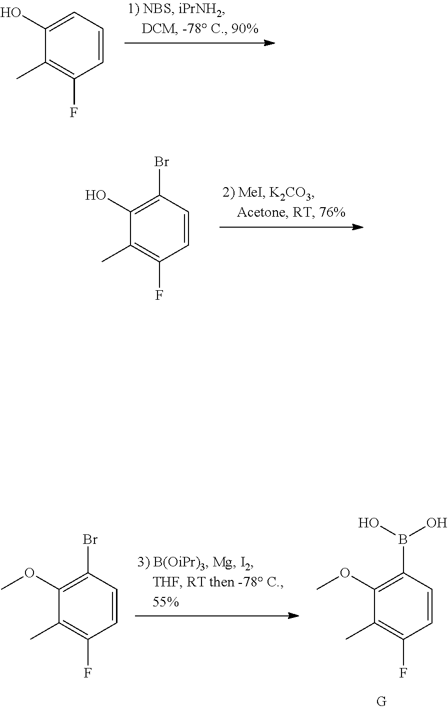

### Step 1:

Isopropylamine (23.460 g, 34.5 mL, 396.89 mmol) was slowly added to a stirred solution of 3-fluoro-2-methylphenol (50 g, 396.42 mmol) in DCM (2.5 L). The reaction mixture was cooled to −78° C. NBS (70 g, 393.29 mmol) was added portionwise over 2 h 10 min and the mixture was stirred for a further 30 min. The mixture was warmed up to 25° C. 2N HCl (500 ml) was added and the mixture was stirred for 15 min. The organic layer was separated and concentrated in vacuo, keeping the water bath at 15° C. Hexane (500 ml) was added to the residue and the mixture was stirred for 10 min. The mixture was filtered and the liquors were concentrated in vacuo, keeping the water bath at 15° C. to give 6-bromo-3-fluoro-2-methylphenol (73 g, 90%) as a light brown oil. ¹H NMR (400 MHZ, Chloroform-d) δ 7.24-7.21 (m, 1H), 6.55 (t, J=8.8 Hz, 1H), 5.61 (s, 1H), 2.20 (s, 3H) ppm.

### Step 2:

To a stirred solution of 6-bromo-3-fluoro-2-methylphenol (40 g, 195.10 mmol) in acetone (400 mL) at ambient temperature was added potassium carbonate (135 g, 976.80 mmol). The reaction mixture was stirred for 10 min at 25° C. Methyl iodide (39 g, 17.105 mL, 274.77 mmol) was added dropwise over 10 min and the mixture was stirred for 16 h at 25° C. The reaction mixture was filtered and the solid residues washed with acetone (50 ml). The mother liquors were concentrated at 15° C. under reduced pressure. Hexane (200 ml) was added and the mixture was stirred for 15 min. The solid was collected and washed with hexane (8 ml). The mother liquors were concentrated under reduced pressure at 15° C. Purification by distillation (520 mm Hg, 192-196° C.) gave 1-bromo-4-fluoro-2-methoxy-3-methylbenzene (32.4 g, 76%). ¹H NMR (400 MHz, Chloroform-d) δ 7.33-7.30 (m, 1H), 6.72 (t, J=8.7 Hz, 1H), 3.80 (s, 3H), 2.23 (s, 3H) ppm.

### Step 3:

Iodine (50 mg, 0.1970 mmol) was added at 25° C. to a stirred mixture of Mg turnings (5 g, 205.72 mmol) in THF (50 ml). The mixture was stirred until the reaction turned into a clear pale yellow colour. 1-bromo-4-fluoro-2-methoxy-3-methylbenzene (2.5 g, 11.4 mmol) was added dropwise at ambient temperature. When reaction initiation was observed, the remaining solution of 1-bromo-4-fluoro-2-methoxy-3-methylbenzene (22.5 g, 102.71 mmol) in THF (200 ml) was added dropwise. The mixture was stirred for 40 min. The reaction mixture was cooled to −78° C. and triisopropylborate (64.385 g, 79 mL, 342.34 mmol) was added dropwise. The mixture was warmed up to ambient temperature and stirred for 16 h. The reaction was quenched by addition of 2N HCl (25 ml) and stirred for 15 min. The mixture was diluted with water (125 ml) and extracted with ethyl acetate (2×250 ml). The organic layer was separated, washed with water (250 ml), dried over Na₂SO₄, filtered, and concentrated in vacuo. Hexane (2 5 ml) was added to the residue at 0° C. and the mixture was stirred for 5 min. The resulting solid was filtered, washed with 10 ml of chilled hexane and dried to give (4-fluoro-2-methoxy-3-methylphenyl) boronic acid (11.5 g, 55%). ¹H NMR (400 MHZ, DMSO-d₆) δ 7.96 (br s, 2H), 7.32 (t, J=8.0 Hz, 1H), 6.88 (t, J=8.7 Hz, 1H), 3.75 (s, 3H), 2.11 (s, 3H) ppm.

### Intermediate H

### rac-4-((2S,3R,5S)-3-(4-fluoro-2-methoxy-3-methylphenyl)-5-methyl-5-(trifluoromethyl)tetrahydrofuran-2-carboxamido)picolinamide

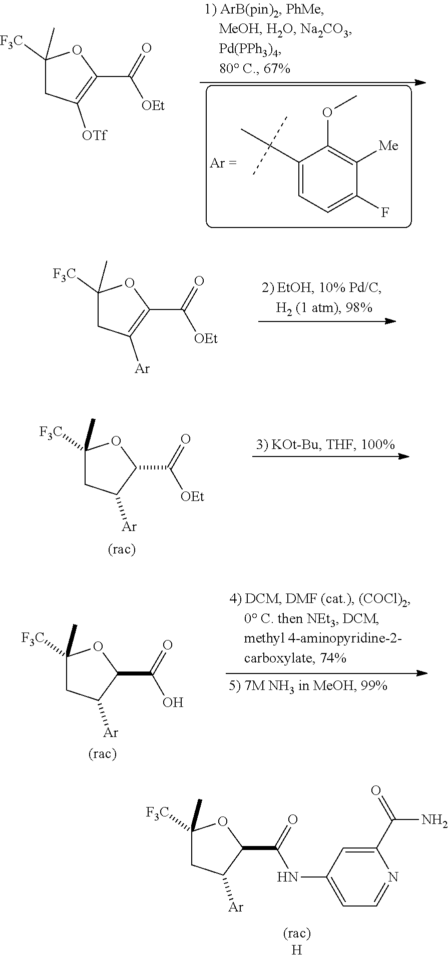

### Step 1:

A solution of ethyl 2-methyl-2-(trifluoromethyl)-4-(trifluoromethylsulfonyloxy)-3H-furan-5-carboxylate (4.958 g, 13.32 mmol), 2-(4-fluoro-2-methoxy-3-methyl-phenyl)-4,4,5,5-tetramethyl-1,3,2-dioxaborolane (4.18 g, 15.71 mmol), Pd(PPh₃)₄ (690 mg, 0.5971 mmol) and Na₂CO₃ (3.55 g, 33.49 mmol) in PhMe (75 mL), MeOH (7.5 mL) and H₂O (7.5 mL) was degassed, then heated at 80° C. for 16 h. The reaction was cooled to ambient temperature and diluted with EtOAc. The layers were separated and the organic phase was washed with brine, dried over MgSO₄, filtered, and concentrated in vacuo. Purification by flash chromatography (80 g SiO₂, 0 to 20% EtOAc in heptane) gave ethyl 3-(4-fluoro-2-methoxy-3-methylphenyl)-5-methyl-5-(trifluoromethyl)-4,5-dihydrofuran-2-carboxylate (3.24 g, 67%) as a yellow oil. ESI-MS m/z calc. 362.11414, found 463.4 (M+1)⁺; Retention time: 1.05 minutes.

### Step 2:

EtOH (110 mL) was added to ethyl 3-(4-fluoro-2-methoxy-3-methylphenyl)-5-methyl-5-(trifluoromethyl)-4,5-dihydrofuran-2-carboxylate (3.24 g, 8.942 mmol) and Pd/C (1.5 g of 10% w/w, 1.410 mmol). The mixture was degassed and stirred overnight under a balloon of hydrogen. The mixture was filtered through Celite, washing with EtOH, and concentrated in vacuo to give ethyl rac-(2S,3S,5R)-3-(4-fluoro-2-methoxy-3-methylphenyl)-5-methyl-5-(trifluoromethyl)tetrahydrofuran-2-carboxylate (3.19 g, 98%). ESI-MS m/z calc. 364.12976, found 365.4 (M+1)⁺; Retention time: 1.07 minutes.

### Step 3:

A solution of ethyl rac-(2S,3S,5R)-3-(4-fluoro-2-methoxy-3-methylphenyl)-5-methyl-5-(trifluoromethyl)tetrahydrofuran-2-carboxylate (783 mg, 2.149 mmol) and KOt-Bu (720 mg, 6.416 mmol) in THF (22 mL) was stirred overnight at ambient temperature. The reaction was diluted in EtOAc and quenched by addition of 1 M HCl. The layers were separated and the aqueous layer was extracted with EtOAc. The combined organic extracts were dried over MgSO₄, filtered, and concentrated in vacuo to give rac-(2R,3S,5R)-3-(4-fluoro-2-methoxy-3-methylphenyl)-5-methyl-5-(trifluoromethyl)tetrahydrofuran-2-carboxylic acid (722.7 mg) as an orange residue, which was taken on to the next step without further purification.

### Step 4:

To a solution of rac-(2R,3S,5R)-3-(4-fluoro-2-methoxy-3-methylphenyl)-5-methyl-5-(trifluoromethyl)tetrahydrofuran-2-carboxylic acid (923.1 mg, 2.745 mmol) in DCM (20 mL) at 0° C. was added DMF (25 μL, 0.3229 mmol) and oxalyl chloride (720 μL, 8.254 mmol). The reaction was stirred for 30 min. Further amounts of oxalyl chloride (720 μL, 8.254 mmol) and DMF (25 μL, 0.3229 mmol) were added to the reaction mixture. The reaction was stirred for 30 min before being concentrated in vacuo. The residue was diluted in DCM (18 mL) and added dropwise to a solution of methyl 4-aminopyridine-2-carboxylate (605 mg, 3.976 mmol), DMAP (25 mg, 0.2046 mmol) and Et₃N (2.5 mL, 17.94 mmol) in DCM (15 mL). The reaction was stirred at 0° C. for 10 min, then warmed to ambient temperature and stirred for a further 16 h. The reaction mixture was diluted in DCM and washed with 1 M HCl solution. The organic layer was separated, dried over MgSO₄, filtered and concentrated in vacuo. Purification by flash chromatography (40 g SiO₂, 0 to 100% EtOAc in heptane) gave methyl rac-4-((2R,3S,5R)-3-(4-fluoro-2-methoxy-3-methylphenyl)-5-methyl-5-(trifluoromethyl)tetrahydrofuran-2-carboxamido) picolinate (801 mg, 62%). ESI-MS m/z calc. 470.14648, found 471.5 (M+1)⁺; 469.6 (M−1); Retention time: 0.94 minutes.

### Step 5:

Methyl rac-4-((2R,3S,5R)-3-(4-fluoro-2-methoxy-3-methylphenyl)-5-methyl-5-(trifluoromethyl)tetrahydrofuran-2-carboxamido) picolinate (800 mg, 1.701 mmol) was dissolved in methanolic ammonia (15 mL of 7 M, 105.0 mmol) and the solution was stirred at ambient temperature overnight. The mixture was concentrated in vacuo to give rac-4-((2R,3S,5R)-3-(4-fluoro-2-methoxy-3-methylphenyl)-5-methyl-5-(trifluoromethyl)tetrahydrofuran-2-carboxamido) picolinamide (767 mg, 99%) ESI-MS m/z calc. 455.14682, found 456.5 (M+1)⁺; 454.6 (M−1); Retention time: 0.89 minutes.

### Intermediate I

### 1-Bromo-4-fluoro-2-methoxy-3-(methoxymethyl)benzene

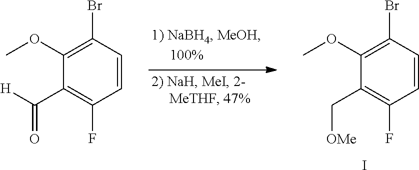

### Step 1:

NaBH₄ (449 mg, 11.87 mmol) was added to an ice cold solution of 3-bromo-6-fluoro-2-methoxybenzaldehyde (2.93 g, 12.57 mmol) in MeOH (50 mL). The reaction mixture was stirred for 10 min. The mixture was quenched by addition of 1 M HCl and diluted with EtOAc. The aqueous layer was separated and washed with EtOAc (2×). The combined organic extracts were dried over MgSO₄, filtered, and concentrated in vacuo to give (3-bromo-6-fluoro-2-methoxyphenyl) methanol (3.04 g, 100%). 1H NMR (400 MHZ, Methanol-d₄) δ 7.55 (dd, J=8.9, 6.1 Hz, 1H), 6.89 (t, J=8.9 Hz, 1H), 4.69 (d, J=1.9 Hz, 2H), 3.91 (s, 3H) ppm. ESI-MS m/z calc. 233.96918, found 218.6 (M-F)⁺; Retention time: 0.65 minutes.

### Step 2:

NaH (25 mg of 60% w/w, 0.6251 mmol) was added to a solution of (3-bromo-6-fluoro-2-methoxyphenyl) methanol (100 mg, 0.4254 mmol) in 2-MeTHF (2 mL). The reaction mixture was stirred for 15 min before adding Mel (45 μL, 0.7228 mmol). The mixture was stirred at ambient temperature for 4 h. A further amount of Mel (45 μL, 0.7228 mmol) was added and the mixture was stirred at ambient temperature overnight. The reaction mixture was quenched by addition of a saturated aqueous NH₄Cl solution. The mixture was diluted with water and EtOAc. The mixture was filtered using a Whatman 1PS hydrophobic phase separator filter paper. The organic extracts were concentrated in vacuo. Purification by flash chromatography (SiO₂, 0 to 50% EtOAc in heptane) gave 1-bromo-4-fluoro-2-methoxy-3-(methoxymethyl)benzene (50 mg, 47%) as a colourless oil. ¹H NMR (400 MHZ, Chloroform-d) δ 7.49 (dd, J=8.9, 6.1 Hz, 1H), 6.80 (t, J=8.7 Hz, 1H), 4.53 (d, J=2.1 Hz, 2H), 3.91 (s, 3H), 3.41 (d, J=0.6 Hz, 3H) ppm. ESI-MS m/z calc. 247.98482, found 248.9 (M−1); Retention time: 0.84 minutes.

### Intermediate J

### 4-bromo-2,2,7-trifluorobenzo[d][1,3]dioxole

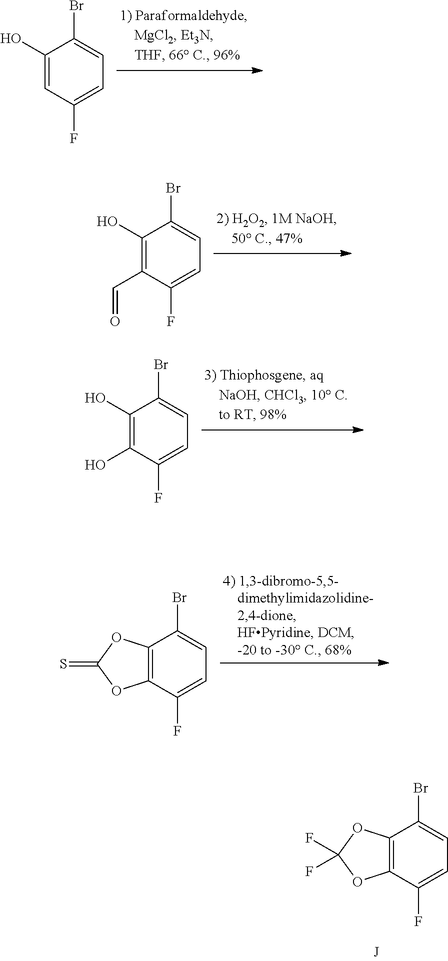

### Step 1:

Triethylamine (23.232 g, 32 mL, 229.59 mmol) was added to a mixture of paraformaldehyde (20 g, 322.23 mmol) and MgCl2 (20 g, 210.06 mmol) in THF (500 mL) under argon. The reaction mixture was stirred at room temperature for 10 min before adding 2-bromo-5-fluorophenol (20 g, 104.71 mmol). The reaction was heated to reflux for 2 h then allowed to cool to ambient temperature. Diethyl ether (700 mL) was added and the organic phase was washed with a 1N HCl solution (3×400 mL), dried over Na₂SO₄, filtered, and concentrated in vacuo to give 3-bromo-6-fluoro-2-hydroxybenzaldehyde (22 g, 96%), which was used as is in the next step.

### Step 2:

H₂O₂ (25.530 g, 23 mL of 30% w/v, 750.56 mmol) was added to a solution of 3-bromo-6-fluoro-2-hydroxybenzaldehyde (22 g, 100.45 mmol) in NaOH (120 mL of 1 M, 120.00 mmol) while applying external cooling to keep the temperature below 50° C. The reaction mixture was stirred for 2 hr. The reaction was quenched by pouring the mixture in a NaHSO₃ solution (120 ml). The resulting mixture was extracted with ethyl acetate (500 ml). The organic phase was washed with a saturated NaCl solution (100 ml), dried over Na₂SO₄, filtered, and concentrated in vacuo. Purification by flash chromatography (SiO₂, 0 to 5% ethyl acetate in hexane) gave 3-bromo-6-fluorobenzene-1,2-diol (9.8 g, 47%) as a yellow/orange oil. ¹H NMR (400 MHZ, DMSO-d₆) δ 9.82 (d, J=8.6 Hz, 1H), 9.65 (s, 1H), 6.94 (dd, J=9.0, 5.7 Hz, 1H), 6.64 (t, J=9.6 Hz, 1H) ppm.

### Step 3:

A mixture of 3-bromo-6-fluorobenzene-1,2-diol (2.3 g, 11.111 mmol) and thiophosgene (1.5 g, 1 mL, 13.046 mmol) in chloroform (15 mL) was cooled down to 10° C. NaOH (10 mL of 10% w/v, 25.002 mmol) was added dropwise over 30 min under vigorous stirring. The reaction mixture was stirred for 2 hr at ambient temperature. The mixture was concentrated in vacuo. The residue was dissolved in ethyl acetate (200 mL). The organic solution was washed with water (30 mL) and a saturated NaCl solution (30 mL), dried over Na₂SO₄, filtered, and concentrated in vacuo to give 4-bromo-7-fluorobenzo[d][1,3]dioxole-2-thione (2.7 g, 98%) as an off-white solid. ¹H NMR (400 MHZ, Chloroform-d) δ 7.66 (dd, J=9.2, 4.2 Hz, 1H), 7.1 (t, J=9.6 Hz, 1H) ppm.

### Step 4:

Pyridine hydrofluoride (9 mL of 70% w/v, 63.568 mmol) was added to a solution of 4-bromo-7-fluorobenzo[d][1,3]dioxole-2-thione (3.3 g, 13.250 mmol) in DCM (35 mL) cooled down to −30° C. 1,3-Dibromo-5,5-dimethylimidazolidine-2,4-dione (4.6 g, 16.088 mmol) was added portionwise over 30 min. The mixture was stirred for 2 hr between-20 to −30° C. The reaction mixture was quenched by pouring the mixture in a 5% NaHSO₃ solution (9 ml). The mixture was stirred for 10 min. The organic layer was separated, dried over Na₂SO₄, filtered, and concentrated in vacuo. Purification by flash chromatography (SiO₂, 100% hexane) gave 4-bromo-2,2,7-trifluorobenzo[d][1,3]dioxole (2.3 g, 68%) as a colourless oil. ¹H NMR (400 MHZ, Chloroform-d) δ 7.16 (dd, J=9.2, 4.0 Hz, 1H), 6.84 (t, J=9.2 Hz, 1H) ppm.

### Intermediate K

### 1-bromo-4-(cyclopropylmethoxy)-3-fluoro-2-methoxybenzene

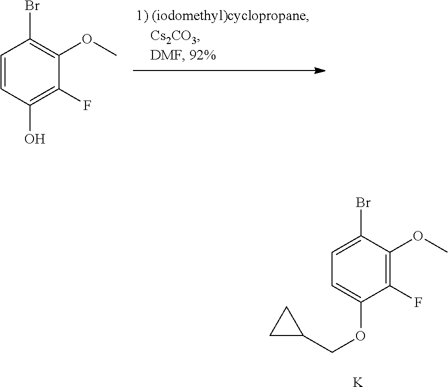

### Step 1:

(Iodomethyl)cyclopropane (3.6 g, 19.78 mmol) was added to a mixture of 4-bromo-2-fluoro-3-methoxyphenol (1 g, 4.524 mmol) and cesium carbonate (2.2 g, 6.752 mmol) in DMF (20 mL). The reaction mixture was stirred at ambient temperature for 48 h. The mixture was partitioned between MTBE (10 ml) and water (10 ml). The aqueous layer was separated and extracted with MTBE (10 mL). The combined organic extracts were washed with brine (1×10 mL), dried over MgSO₄, filtered, and concentrated in vacuo to give 1-bromo-4-(cyclopropylmethoxy)-3-fluoro-2-methoxybenzene (1.15 g, 92%). ESI-MS m/z calc. 274.00046, Retention time: 1.01 minutes; no mass ionisation.

### Intermediate L

### 1-bromo-3,4-difluoro-2-(methoxymethyl)benzene

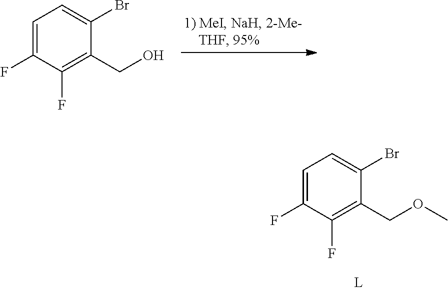

### Step 1:

(6-Bromo-2,3-difluorophenyl) methanol (2 g, 8.968 mmol) was added to a suspension of sodium hydride (400 mg, 60% w/w in mineral oil, 10.001 mmol) in 2-methyltetrahydrofuran (20 mL) at 0° C. The mixture was stirred at 0° C. for 30 min before adding methyl iodide (1.4 g, 0.614 mL, 9.863 mmol). The reaction mixture was stirred at room temperature for 4 h. The mixture was quenched by addition of water (10 mL). The organic layer was separated, washed with water (10 mL), brine (10 mL), dried over Na₂SO₄, filtered, and concentrated in vacuo to give 1-bromo-3,4-difluoro-2-(methoxymethyl)benzene (2.05 g, 95%) as a colourless oil. ¹H NMR (300 MHz, Chloroform-d) δ 7.34 (ddd, J=8.9, 4.5, 2.1 Hz, 1H), 7.05 (ddd, J=8.9, 8.9, 8.3 Hz, 1H), 4.62 (d, J=2.8 Hz, 2H), 3.42 (s, 3H) ppm.

### Intermediate M

### 4-bromo-1,1,7-trifluoro-2,3-dihydro-1H-indene

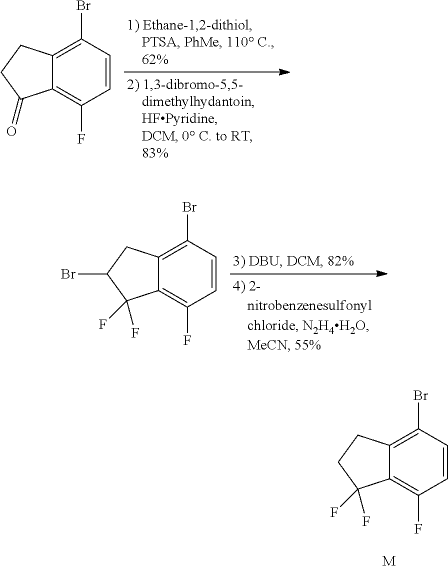

### Step 1:

Ethane-1,2-dithiol (2.123 g, 1.89 mL, 22.532 mmol) and PTSA (700 mg, 4.065 mmol) were successively added to a stirred solution of 4-bromo-7-fluoro-2,3-dihydro-1H-inden-1-one (4.7 g, 20.520 mmol) in toluene (50 mL). The reaction mixture was heated at reflux for 72 h using a Dean-Stark apparatus. After cooling to ambient temperature, the reaction mixture was washed with 10% NaOH (50 mL). The aqueous layer was separated and extracted with DCM (2×20 mL). The combined organic extracts were concentrated in vacuo. Purification by flash chromatography (SiO₂, 0 to 5% ethyl acetate in heptane) gave 4-bromo-7-fluoro-2,3-dihydrospiro[indene-1,2′-[1,3]dithiolane] (4.76 g, 62%) as a light brown oil that solidified on standing. ¹H NMR (400 MHZ, Chloroform-d) δ 7.33 (q, J=4.4 Hz, 1H), 6.82-6.77 (m, 1H), 3.61-3.52 (m, 2H), 3.47-3.38 (m, 2H), 3.03-2.97 (m, 2H), 2.77-2.71 (m, 2H) ppm.

### Step 2:

Hydrogen fluoride pyridine complex (660 mg, 0.6 mL of 70% w/v, 6.660 mmol) was added dropwise to a stirred solution of 1,3-dibromo-5,5-dimethylhydantoin (600 mg, 2.099 mmol) in DCM (2.5 mL) cooled in a dry ice/acetone bath. After stirring the mixture for 30 min, a solution of 4-bromo-7-fluoro-2,3-dihydrospiro[indene-1,2′-[1,3]dithiolane] (200 mg, 0.531 mmol) in DCM (1 mL) was added dropwise to the reaction mixture. The reaction mixture was stirred for 1 h and the cooling bath was removed. The mixture was warmed up to ambient temperature. The mixture was poured into a mixture of 2 M aqueous NaOH (10 mL) and saturated aqueous sodium bisulfite (1.5 mL). The mixture was extracted with DCM (2×10 mL). The combined organic extracts were dried over MgSO₄, filtered, and concentrated in vacuo to give 2,4-dibromo-1,1,7-trifluoro-2,3-dihydro-1H-indene (181 mg, 83%) as a brown oil. ¹H NMR (400 MHZ, Chloroform-d) δ 7.64-7.59 (m, 1H), 6.98 (q, J=8.5 Hz, 1H), 4.64-4.55 (m, 1H), 3.68-3.55 (m, 1H), 3.25-3.19 (m, 1H) ppm. ¹⁹F NMR (376 MHZ, Chloroform-d) δ −88.6 (d, J=250.2 Hz, 1F), −96.4 (d, J=250.0 Hz, 1F), −118.9-−119.1 (m, 1F) ppm.

### Step 3:

DBU (1.323 g, 1.3 mL, 8.693 mmol) was added dropwise to a stirred solution of 2,4-dibromo-1,1,7-trifluoro-2,3-dihydro-1H-indene (2 g, 5.759 mmol) in DCM (25 mL). The reaction mixture was stirred at ambient temperature for 3 h. The mixture was washed with a 2 M HCl (2×25 mL). The combined aqueous phases were extracted with DCM (2×25 mL). The combined organic extracts were washed with brine, dried over MgSO₄, filtered, and concentrated in vacuo to give a brown oil. Purification by flash chromatography (SiO₂, 0 to 5% ethyl acetate in heptane) gave 4-bromo-1,1,7-trifluoro-1H-indene (1.244 g, 82%) as a green oil. ¹H NMR (400 MHZ, Chloroform-d) δ 7.43 (q, J=4.4 Hz, 1H), 6.86-6.81 (m, 2H), 6.26 (d, J=6.0 Hz, 1H) ppm. 1° F. NMR (376 MHZ, Chloroform-d) δ −119.9-−119.9 (m, 1F), −122.4-−122.4 (m, 2F) ppm.

### Step 4:

Hydrazine hydrate (800 mg, 0.8 mL, 15.981 mmol) was slowly added to a solution of 4-bromo-1,1,7-trifluoro-1H-indene (1.1 g, 3.976 mmol) and 2-nitrobenzenesulfonyl chloride (1.8 g, 8.122 mmol) in acetonitrile (40 mL) at 0° C. The reaction mixture was stirred at ambient temperature for 18 h. The mixture was partitioned between ethyl acetate (250 ml) and water (60 mL). The organic layer was separated, dried over Na₂SO₄, filtered, and concentrated in vacuo to give a brown gum. Purification by flash chromatography (SiO₂, 10% ethyl acetate in heptane) gave 4-bromo-1,1,7-trifluoro-2,3-dihydro-1H-indene (0.65 g, 55%) as a pale yellow oil. ¹H NMR (400 MHZ, Chloroform-d) δ 7.56 (q, J=4.4 Hz, 1H), 6.91 (t, J=8.7 Hz, 1H), 3.04-2.99 (m, 2H), 2.70-2.59 (m, 2H) ppm. ¹⁹F NMR (376 MHZ, Chloroform-d) δ −84.2-−84.3 (m, 2F), −120.0-−120.1 (m, 1F) ppm.

### Intermediate N

### Methyl 4-amino-5-methylpicolinate

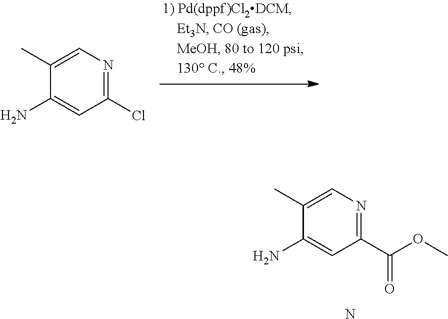

### Step 1:

In an autoclave (600 mL), Pd(dppf)Cl₂·DCM (3.4 g, 4.1634 mmol) and triethylamine (29.040 g, 40 mL, 286.98 mmol) were added to a mixture of 2-chloro-5-methylpyridin-4-amine (20 g, 140.27 mmol) in methanol (158.20 g, 200 mL, 4.937 mol). The autoclave was purged with nitrogen, then with carbon monoxide. The pressure was adjusted to 80 psi and the mixture was heated to 130° C. The carbon monoxide pressure was adjusted to 120 psi. The mixture was stirred 5 h at 130° C., then cooled down to 20° C. and stirred overnight. The mixture was purged with nitrogen and concentrated in vacuo. Purification by flash chromatography (110 g SiO₂, 0 to 10% MeOH in ethyl acetate) gave the crude product, which was triturated with MTBE (50 mL) for 1 h at 22° C. The solid was filtered, washed with TBME (25 mL), dried in vacuo at 50° C. for 3 h to give methyl 4-amino-5-methylpicolinate (12.05 g, 48%) as a beige solid. ESI-MS m/z calc. 166.0742, found 167.1 (M+1)⁺; Retention time: 0.512 minutes.

### Intermediate O

### Methyl 4-chloro-5-(trimethylsilyl) picolinate

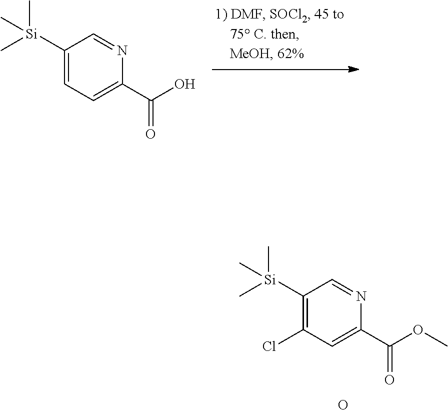

### Step 1:

DMF (141.60 mg, 0.15 mL, 1.937 mmol) was added to thionyl chloride (4.893 g, 3 mL, 41.128 mmol) at 45° C. before adding 5-(trimethylsilyl)picolinic acid (1.25 g, 1.5701 mmol) portionwise. The reaction mixture was heated at 75° C. for 3 days then cooled to ambient temperature. Toluene (5 mL) was added and the mixture was concentrated in vacuo. Methanol (5 mL, 123.43 mmol) was added and the mixture was stirred at ambient temperature for 30 min. The mixture was concentrated in vacuo. The residue was partitioned between a saturated aqueous sodium hydrogencarbonate solution (5 mL) and ethyl acetate (5 mL). The aqueous phase was separated and extracted with ethyl acetate (3×5 mL). The combined organic extracts were dried over Na₂SO₄, filtered, and concentrated in vacuo. Purification by flash chromatography (25 g SiO₂, 0 to 100% ethyl acetate in heptane) followed by reverse phase chromatography (25 g SiO₂, 0 to 100% acetonitrile containing 0.1% ammonium hydroxide in water containing 0.1% ammonium hydroxide) gave methyl 4-chloro-5-(trimethylsilyl) picolinate (79 mg, 13%) as a yellow oil. ¹H NMR (400 MHZ, Chloroform-d) δ 8.67 (s, 1H), 8.06 (s, 1H), 4.00 (s, 3H), 0.42 (s, 9H) ppm. ESI-MS m/z calc. 243.0482, found 244.04 (M+1)⁺; Retention time: 1.02 minutes.

### Intermediate P

### (R)-2-(2-oxa-6-azaspiro[3.3]heptan-6-yl) propan-1-ol

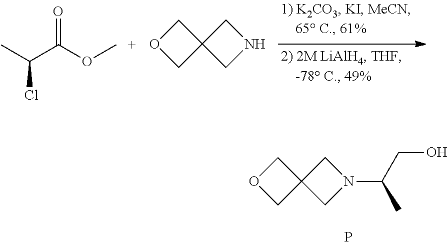

### Step 1:

Potassium carbonate (11 g, 79.591 mmol) was added to a suspension of 2-oxa-6-azaspiro[3.3]heptane (4 g, 40.351 mmol), methyl(S)-2-chloropropanoate (4.95 g, 40.392 mmol) and potassium iodide (0.5 g, 3.0120 mmol) in acetonitrile (50 mL). The mixture was stirred at 65° C. for 15 h. The reaction mixture was partitioned between ethyl acetate (100 mL) and water (25 ml). The organic layer was separated and washed with brine (20 ml), dried (Na₂SO₄), filtered and concentrated in vacuo to give methyl (R)-2-(2-oxa-6-azaspiro[3.3]heptan-6-yl) propanoate (5.1 g, 61%) as a yellow oil. 1H NMR (301 MHZ, Chloroform-d) δ 4.79-4.71 (m, 4H), 3.70 (s, 3H), 3.44 (d, J=7.2 Hz, 4H), 2.99 (q, J=6.9 Hz, 1H), 1.18 (d, J=6.9 Hz, 3H) ppm.

### Step 2:

2 M LiAlH₄ (7.5 mL, 2 M in THF, 15.0 mmol) was added slowly to a solution of methyl (R)-2-(2-oxa-6-azaspiro[3.3]heptan-6-yl) propanoate (1.5 g, 7.289 mmol) in THF (10 mL) at −78° C. The reaction was stirred at −78° C. for 1 h. The mixture was quenched at −78° C. by addition of sodium sulfate decahydrate. The mixture was warmed up to ambient temperature over 6 h and filtered through a pad of celite, rinsing with ethyl acetate (30 mL). The filtrate was concentrated in vacuo to give (R)-2-(2-oxa-6-azaspiro[3.3]heptan-6-yl) propan-1-ol (620 mg, 49%) as a colourless oil, which solidified on standing. 1H NMR (400 MHZ, Chloroform-d) δ 4.74 (d, J=11.4 Hz, 4H), 3.49 (dd, J=10.8, 3.9 Hz, 1H), 3.40-3.31 (m, 5H), 2.32-2.25 (m, 1H), 2.08-1.90 (br s, 1H), 0.94 (d, J=6.4 Hz, 3H) ppm.

The following intermediates were made using a method similar to that described in Intermediate P except that methyl (R)-2-chloropropanoate was used in place of methyl(S)-2-chloropropanoate in Step 1:

| Cmpd No. | Compound Name | NMR (shifts in ppm) |
|---|---|---|
| P-1 | (S)-2-(2-oxa-6- | ¹H NMR (400 MHz, |
| | azaspiro[3.3]heptan-6- | Chloroform-d) δ 4.74 (d, |
| | yl)propan-1-ol | J = 11.7 Hz, 4H), 3.50 |
| | | (dd, J = 11.0, 4.1 Hz, |
| | | 1H), 3.40-3.31 (m, 5H), |
| | | 2.32-2.25 (m, 1H), 2.05- |
| | | 2.01 (br s, 1H), 0.94 (d, |
| | | J = 6.4 Hz, 3H) ppm. |

### Intermediate Q

### (E)-2-(((tert-butyldimethylsilyl)oxy)methyl) but-2-en-1-ol

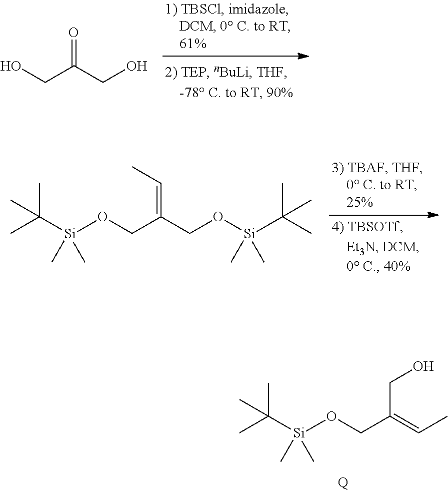

### Step 1:

tert-Butylchlorodimethylsilane (4 g, 26.54 mmol) was added to an ice cold solution of 1,3-dihydroxypropan-2-one (1 g, 11.10 mmol) and imidazole (2 g, 29.38 mmol) in DCM (30 mL) under nitrogen. The solution was stirred for 18 h gradually warming up to ambient temperature. The mixture was quenched addition of water and diluted with DCM. The mixture was stirred for 5 min, the organic was isolated by passing the solution through a phase separator cartridge. The filtrates were concentrated in vacuo. Purification by flash chromatography (40 g SiO₂, 0 to 100% EtOAc in hexanes) gave 1,3-bis((tert-butyl(dimethyl)silyl)oxy) propan-2-one (2.15 g, 61%) as a colourless oil. ¹H NMR (500 MHZ, Cholroform-d) δ 4.32 (s, 4H), 0.83 (s, 18H), 0.00 (s, 12H) ppm.

### Step 2:

A solution of ⁿBuLi (8.5 mL, 1.6 M in hexanes, 13.60 mmol) was added to a solution of TEP (5 g, 13.47 mmol) in THF (30 mL) at −78° C. under nitrogen. The solution was warmed up to ambient temperature for 20 min. The mixture was cooled again to −78° C. before slowly adding a solution of 1,3-bis((tert-butyl(dimethyl)silyl)oxy) propan-2-one (2.15 g, 6.748 mmol) in tetrahydrofuran (˜ 5 mL). The reaction mixture was warmed up to ambient temperature over 1 h and quenched by addition of water (100 mL). The mixture was extracted with pentane (3×100 mL). The combined organic extracts were washed with water (2×50 mL) and brine (100 mL), dried (MgSO₄), filtered and concentrated in vacuo. Purification by flash chromatography (40 g SiO₂, 3% EtOAc in hexanes) gave tert-butyl-(2-((tert-butyl (dimethyl)silyl)oxymethyl) but-2-enoxy)dimethylsilane (2 g, 90%) as a colourless oil. ¹H NMR (500 MHz, Chloroform-d) δ 5.56 (q, 1H), 4.21 (s, 2H), 4.16 (s, 2H), 1.67 (d, 3H), 0.90 (s, 9H), 0.89 (s, 9H), 0.06 (s, 12H) ppm.

### Step 3:

TBAF (4.2 mL, 1 M in THF, 4.2 mmol) was added to an ice cold solution of tert-butyl-(2-((tert-butyl(dimethyl)silyl)oxymethyl) but-2-enoxy)dimethylsilane (650 mg, 1.966 mmol) in THF (5 mL) under nitrogen. Five minutes after the end of addition, the ice bath was removed and the solution was stirred at ambient temperature for 1 h. The reaction mixture was diluted with EtOAc, washed with a saturated aqueous sodium bicarbonate solution and brine. The organic phase was dried (MgSO₄), filtered and concentrated in vacuo. Purification by flash chromatography (4 g SiO₂, 0 to 100% EtOAc in hexanes) gave 2-ethylidenepropane-1,3-diol (50 mg, 25%) as a colourless oil. ¹H NMR (500 MHz, Chloroform-d) δ 5.60-5.58 (m, 1H), 4.26 (d, 2H), 4.06 (d, 2H), 2.57-2.47 (m, 2H), 1.64 (d, 3H).

### Step 4:

tert-Butyldimethylsilyl trifluoromethanesulfonate (165 μL, 0.719 mmol) was added dropwise to an ice cold solution of 2-ethylidenepropane-1,3-diol (70 mg, 0.685 mmol) and Et₃N (150 μL, 1.076 mmol) in DCM (3 mL) under nitrogen. The reaction mixture was stirred with the ice bath in place for 2 h. The suspension was diluted with DCM and water and stirred for 5 min. The organic phase was isolated by passing the solution through a phase separator cartridge. The filtrates were concentrated in vacuo. Purification by flash chromatography (12 g SiO₂, 0 to 100% EtOAc in hexanes) gave (E)-2-(((tert-butyldimethylsilyl)oxy)methyl) but-2-en-1-ol (60 mg, 40%) as a colourless oil. ¹H NMR (500 MHZ, Chloroform-d) δ 5.50-5.47 (m, 1H), 4.28 (s, 1H), 4.16-4.13 (d, 2H), 4.05-4.01 (m, 1H), 1.85 (br s, 1H), 1.61-1.55 (m, 3H), 0.83 (s, 9H), 0.00 (m, 6H) ppm.

The following intermediates were made using a method similar to that described in Intermediate Q except that, in step 1, different starting materials were used in place of 1,3-dihydroxypropan-2-one. Steps 2 to 4 were omitted:

| Cmpd No. | Compound Name | NMR (shifts in ppm) |
|---|---|---|
| Q-1 | (1R,3R)-cyclopentane-1,3-diol | ¹H NMR (500 MHz, |
| | | DMSO-d₆) δ 4.41 (d, J = |
| | | 3.9 Hz, 1H), 4.35 (tt, J = |
| | | 6.3, 4.5 Hz, 1H), 4.18 (tq, |
| | | J = 6.0, 3.9 Hz, 1H), 2.01- |
| | | 1.81 (m, 2H), 1.73- |
| | | 1.65 (m, 1H), 1.65-1.57 |
| | | (m, 1H), 1.43-1.32 (m, |
| | | 2H), 0.84 (s, 9H), 0.02 (s, |
| | | 6H) ppm. |
| Q-2 | ((1r,3r)-3- | ¹H NMR (500 MHz, |
| | bromocyclobutoxy)(tert- | Chloroform-d) δ 4.79- |
| | butyl)dimethylsilane | 4.71 (m, 1H), 4.44 (tt, J = |
| | | 7.3, 3.5 Hz, 1H), 2.70- |
| | | 2.55 (m, 4H), 0.88 (s, |
| | | 9H), 0.04 (s, 6H). |
| | | ppm. |

### Intermediate R

### 1-methyl-3-((methylsulfonyl)oxy)cyclobutyl acetate

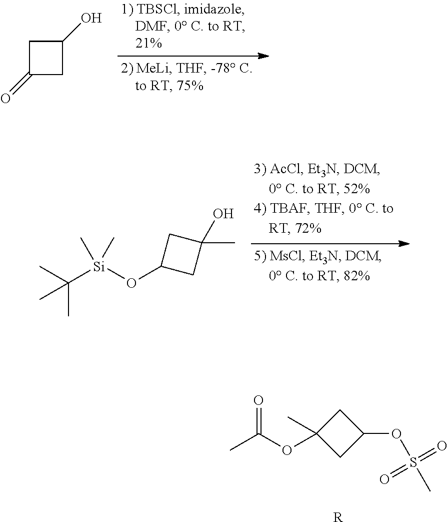

### Step 1:

A solution of 3-hydroxycyclobutanone (2.002 g, 23.25 mmol) in DMF (15 mL) was cooled down in an ice bath. Imidazole (2.359 g, 34.65 mmol) and tert-butylchlorodimethylsilane (4.236 g, 28.10 mmol) were successively added in one portion and the reaction mixture was stirred at 0° C. under nitrogen. An orange precipitate formed. The reaction mixture was warmed to ambient temperature and diluted with DMF (5 mL), causing the precipitate to dissolve. The reaction mixture was stirred under nitrogen at ambient temperature for 18 h. The mixture was diluted with methanol (5 mL), stirred for 30 min and concentrated in vacuo to give an orange oil. Purification by flash chromatography (40 g SiO₂, 0 to 40% EtOAc in heptane) gave 3-((tert-butyldimethylsilyl)oxy)cyclobutan-1-one (1.004 g, 21%) as a clear oil. ¹H NMR (500 MHZ, Chloroform-d) δ 4.60 (tt, J=6.6, 4.8 Hz, 1H), 3.29-3.18 (m, 2H), 3.11-3.02 (m, 2H), 0.91 (s, 9H), 0.09 (s, 6H) ppm.

### Step 2:

Methyllithium (4.4 mL, 1.6 M in Et₂O, 7.040 mmol) was added dropwise over 5 min to a stirred solution of 3-((tert-butyldimethylsilyl)oxy)cyclobutan-1-one (470 mg, 2.322 mmol) in THF (15 mL) at −78° C. under nitrogen. The reaction mixture was warmed up to ambient temperature and stirred under nitrogen for 22 h. The reaction mixture was cooled down to 0° C. and quenched by dropwise addition of methanol (5 mL). The reaction mixture was poured over an aqueous saturated sodium bicarbonate solution (40 mL). The mixture was extracted with EtOAc (3×40 mL). The combined organic extracts were washed with brine (30 mL), dried (MgSO₄), filtered and concentrated in vacuo to give 3-((tert-butyldimethylsilyl)oxy)-1-methylcyclobutan-1-ol (399 mg, 75%) as an orange oil. ¹H NMR (500 MHz, Chloroform-d) δ 3.91 (p, J=6.8 Hz, 1H), 2.47-2.40 (m, 2H), 2.08-2.00 (m, 2H), 1.29 (t, J=0.9 Hz, 3H), 0.88 (s, 9H), 0.03 (s, 6H) ppm; OH alcohol not observed.

### Step 3:

Acetyl chloride (330 μL, 4.641 mmol) was added dropwise over 5 min to a stirred solution of 3-((tert-butyldimethylsilyl)oxy)-1-methylcyclobutan-1-ol (396 mg, 1.830 mmol) and triethylamine (1.1 mL, 7.892 mmol) in DCM (10 mL) at 0° C. under nitrogen. A yellow precipitate formed. The mixture was diluted with DCM (5 mL) and warmed up to ambient temperature over 16 h. The reaction mixture was diluted with an aqueous saturated sodium bicarbonate solution (10 mL) and stirred vigorously for 30 min. The phases were separated and the aqueous layer was extracted with DCM (3×10 mL). The combined organic extracts were passed through a phase separator cartridge. The filtrates were concentrated in vacuo to give an orange oil. Purification by flash chromatography (SiO₂, 0 to 40% EtOAc in heptane) gave 3-((tert-butyldimethylsilyl)oxy)-1-methylcyclobutyl acetate (247 mg, 52%) as a pale yellow oil. ¹H NMR (500 MHz, DMSO-d₆) δ 4.07 (p, J=7.0 Hz, 1H), 2.55 (ddd, J=12.5, 6.1, 2.8 Hz, 2H), 2.16 (s, 3H), 2.08 (tt, J=8.1, 7.2, 2.2 Hz, 2H), 1.41 (s, 3H), 0.85 (s, 9H), 0.02 (d, J=2.6 Hz, 6H) ppm.

### Step 4:

TBAF (1.8 mL of 1 M, 1.8 mmol) was added dropwise over 3 min to a stirred solution of 3-((tert-butyldimethylsilyl)oxy)-1-methylcyclobutyl acetate (235 mg, 0.909 mmol) in THF (5 mL) at 0° C. under nitrogen. The reaction mixture was warmed to ambient temperature and stirred for 3.5 h. TBAF (450 μL of 1 M, 0.450 mmol) was added in one portion and the reaction mixture was left to stir at ambient temperature under nitrogen for 30 min. The reaction mixture was diluted with water (20 mL) and the solution was extracted with EtOAc (3×20 mL). The combined organic extracts were washed with brine (20 mL), dried (MgSO₄), filtered and concentrated in vacuo to give an orange oil. Purification by flash chromatography (12 g SiO₂, 0 to 100% EtOAc in heptane) gave 3-hydroxy-1-methylcyclobutyl acetate (94 mg, 72%) as a clear oil. ¹H NMR (500 MHZ, DMSO-d₆) δ 5.13 (d, J=6.3 Hz, 1H), 3.86 (h, J=7.0 Hz, 1H), 2.49-2.44 (m, 2H), 2.17 (s, 3H), 2.04 (ddd, J=10.1, 7.7, 3.1 Hz, 2H), 1.39 (s, 3H) ppm.

### Step 5:

Methanesulfonyl chloride (100 μL, 1.292 mmol) was added in one portion to a stirred solution of 3-hydroxy-1-methylcyclobutyl acetate (90 mg, 0.624 mmol) and Et₃N (270 μL, 1.937 mmol) in DCM (5 mL) cooled in an ice bath under nitrogen. The reaction mixture was warmed to ambient temperature and stirred under nitrogen for 1.5 h. The mixture was diluted with water (15 mL) and DCM (10 mL) and stirred vigorously for 30 min. The organic phase was isolated by passing the solution through a phase separator cartridge. The filtrates were concentrated in vacuo to give 1-methyl-3-((methylsulfonyl)oxy)cyclobutyl acetate (114 mg, 82%) as an orange oil, which was used without further purification in the next step. ¹H NMR (500 MHZ, DMSO-d₆) δ 4.86 (p, J=7.1 Hz, 1H), 3.17 (s, 3H), 2.77-2.69 (m, 2H), 2.49-2.38 (m, 2H), 2.17 (s, 3H), 1.46 (s, 3H) ppm.

The following intermediate was made using a method similar to that described in Intermediate R except that, in step 1, 2-iodoethan-1-ol was used as the starting material in place of 3-hydroxycyclobutanone. Steps 3 to 5 were omitted:

| Cmpd No. | Compound Name | NMR (shifts in ppm) |
|---|---|---|
| R-1 | (2- | ¹H NMR (500 MHz, |
| | iodoethoxy)triisopropylsilane | DMSO-d₆) δ 3.88 (t, J = |
| | | 6.1 Hz, 2H), 3.32 (t, J = |
| | | 6.1 Hz, 2H), 1.16-0.87 |
| | | (m, 21H) ppm. |

### Intermediate S

### 3-(hydroxymethyl)-1-methylcyclobutan-1-ol

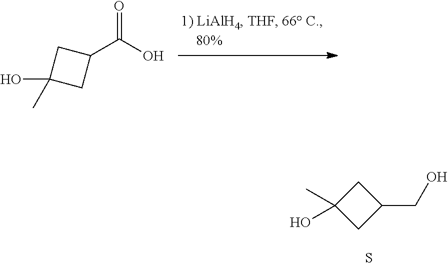

### Step 1:

LiAlH₄ (8 mL, 1 M in THF, 8.0 mmol) was added to a solution of 3-hydroxy-3-methylcyclobutane-1-carboxylic acid (350 mg, 2.689 mmol in THF (8 mL). The reaction mixture was stirred at reflux for 6 h under nitrogen. The mixture was carefully quenched by addition of Na₂SO₄·10H₂O pellets. The mixture was stirred for 30 min before filtering the suspension, washing the filtered cake with more THF. The filtrate was concentrated in vacuo to give 3-(hydroxymethyl)-1-methylcyclobutan-1-ol (250 mg, 80%) as a colourless oil, which was used as is without further purification. ¹H NMR (500 MHz, Chloroform-d) δ 3.79-3.77 (d, 2H), 2.24-2.19 (m, 2H), 2.18-2.11 (m, 1H), 1.89-1.82 (m, 2H), 1.39 (s, 3H) ppm; OH alcohols not observed.

### Intermediate T

### 3-methoxycyclobutyl methanesulfonate

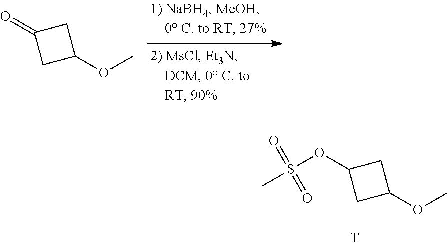

### Step 1:

NaBH₄ (197 mg, 5.207 mmol) was added under nitrogen in one portion to a stirred solution of 3-methoxycyclobutan-1-one (440 mg, 4.395 mmol) in MeOH (13 mL) cooled down with an ice bath. The reaction mixture was gradually warmed up to ambient temperature and stirred for 17 h. The mixture was diluted with water (10 mL) and DCM (15 mL) and left to stir under nitrogen for 2 h. The organic phase was isolated by passing the solution through a phase separator cartridge. The fitrates were concentrated in vacuo to give 3-methoxycyclobutan-1-ol (128 mg, 27%) as a clear oil. ¹H NMR (500 MHz, DMSO-d₆) δ 4.97 (d, J=6.5 Hz, 1H), 3.67 (ddt, J=14.4, 7.9, 6.6 Hz, 1H), 3.35 (tt, J=7.7, 6.5 Hz, 1H), 3.09 (s, 3H), 2.56-2.46 (masked, 2H), 1.65 (dtd, J=8.8, 7.7, 2.9 Hz, 2H) ppm.

### Step 2:

MsCl (260 μL, 3.359 mmol) was added in one portion under nitrogen to a stirred solution of 3-methoxycyclobutan-1-ol (120 mg, 1.116 mmol) and Et₃N (650 μL, 4.664 mmol) in DCM (5 mL) cooled down with an ice bath. The reaction mixture was gradually warmed up to ambient temperature and stirred for 16 h. The mixture was diluted with water (15 mL) and DCM (15 mL) and stirred for 2 h. The organic phase was isolated by passing the solution through a phase separator cartridge. The fitrates were concentrated in vacuo to give 3-methoxycyclobutyl methanesulfonate (200 mg, 90%) as an orange oil. 1H NMR (500 MHZ, Chloroform-d) δ 4.72-4.63 (m, 1H), 3.62-3.53 (m, 1H), 3.25 (s, 3H), 2.99 (s, 3H), 2.90-2.80 (m, 2H), 2.31-2.21 (m, 2H) ppm.

The following intermediate was made using a method similar to that described in Intermediate T except that step 1 was omitted. In step 2, 3,3-difluorocyclobutan-1-ol was used as the starting material in place of 3-methoxycyclobutan-1-ol:

| Cmpd No. | Compound Name | NMR (shifts in ppm) |
|---|---|---|
| T-1 | 3,3-difluorocyclobutyl | ¹H NMR (500 MHz, |
| | methanesulfonate | Chloroform-d) δ 4.97 |
| | | (qtd, J = 7.4, 5.6, 3.2 Hz, |
| | | 1H), 3.15-3.06 (m, 2H), |
| | | 3.05 (s, 3H), 2.99-2.84 |
| | | (m, 2H) ppm. |

### Intermediate U

### 3-((tert-butyldimethylsilyl)oxy)-2-(methoxymethyl) propan-1-ol

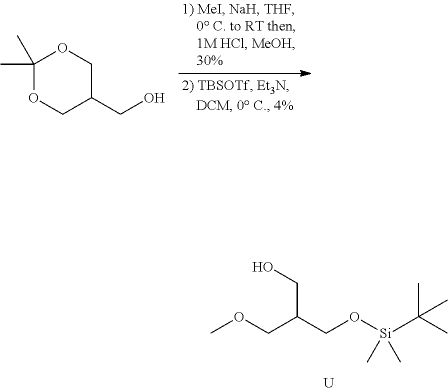

### Step 1:

NaH (360 mg of 60% w/w, 9.001 mmol) was added to an ice cold solution of (2,2-dimethyl-1,3-dioxan-5-yl) methanol (1 g, 6.841 mmol) in THF (15 mL) under nitrogen. The reaction mixture was stirred for 15 min before adding Mel (500 μL, 8.032 mmol). The mixture was gradually warmed up to ambient temperature and stirred for 18 h. The mixture was carefully quenched by addition of water. The mixture was extracted with EtOAc. The organic phase was separated, washed with brine, dried (MgSO₄), filtered and concentrated in vacuo to give a yellow oil. The residue was taken up in a mixture of methanol (5 mL) and 1 M HCl (15 mL, 15.0 mmol) and stirred at ambient temperature for 18 h. The mixture was diluted with DCM and stirred vigorously for 5 min. The organic layer was isolated by passing the solution through a phase separator cartridge. The filtrates were concentrated in vacuo to give 2-(methoxymethyl) propane-1,3-diol (250 mg, 30%) as a yellow oil. ¹H NMR (500 MHZ, Chloroform-d) δ 3.85-3.80 (m, 4H), 3.58-3.57 (dd, 2H), 3.38 (d, 3H), 2.34-2.24 (m, 2H), 2.06-2.01 (m, 1H).

### Step 2:

tert-Butyldimethylsilyl trifluoromethanesulfonate (450 μL, 1.959 mmol) was added dropwise to an ice cold solution of 2-(methoxymethyl) propane-1,3-diol (250 mg, 2.081 mmol) and Et₃N (500 μL, 3.587 mmol) in DCM (5 mL) under nitrogen. The solution was stirred for 2 h before quenching the solution by addition of water. The organic phase was isolated by passing the solution through a phase separator cartridge. The filtrates were concentrated in vacuo to give 3-((tert-butyldimethylsilyl)oxy)-2-(methoxymethyl) propan-1-ol (20 mg, 4%).

The following intermediates were made using a method similar to that described in Intermediate U except that step 1 was omitted. In step 2, different starting materials were used in place of 2-(methoxymethyl) propane-1,3-diol:

| Cmpd No. | Compound Name | NMR (shifts in ppm) |
|---|---|---|
| U-1 | (1r,3r)-3-((tert- | ¹H NMR (500 MHz, |
| | butyldimethylsilyl)oxy)cyclobutan- | Chloroform-d) δ 4.58 (p, |
| | 1-ol | J = 6.2 Hz, 1H), 4.53- |
| | | 4.46 (m, 1H), 2.22 (dd, |
| | | J = 6.2, 5.0 Hz, 4H), 0.88 |
| | | (s, 10H), 0.03 (s, 6H) |
| | | ppm. |
| U-2 | (1s,3s)-3-((tert- | ¹H NMR (500 MHz, |
| | butyldimethylsilyl)oxy)cyclobutan- | Chloroform-d) δ 3.83 |
| | 1-ol | (ddd, J = 13.9, 7.4, 6.4 |
| | | Hz, 2H), 2.75-2.67 (m, |
| | | 2H), 1.89 (dtd, J = 9.3, |
| | | 7.6, 3.0 Hz, 2H), 0.88 (s, |
| | | 9H), 0.04 (s, 6H) ppm. |
| U-3 | 2-(((tert- | ¹H NMR (500 MHz, |
| | butyldimethylsilyl)oxy)methyl)- | Chloroform-d) δ 3.90- |
| | 3,3,3-trifluoropropan-1-ol | 3.80 (m, 4H), 2.41-2.38 |
| | | (m, 1H), 1.80 (s, 1H), |
| | | 0.83 (s, 9H), 0.00 (s, 6H) |
| | | ppm. |

### Intermediate V

### 6-((tert-butyldimethylsilyl)oxy)spiro[3.3]heptan-2-ol

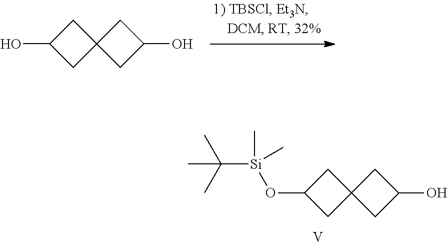

### Step 1:

Et₃N (400 μL, 2.870 mmol) was added to a mixture of spiro[3.3]heptane-2,6-diol (300 mg, 2.341 mmol) and TBSCl (350 mg, 2.322 mmol) in DCM (6 mL). The reaction mixture was stirred at ambient temperature for 18 h. The suspension was diluted with DCM and water. After stirring for 5 minutes, the organic phase was isolated by passing the solution through a phase separator cartridge. The filtrates were concentrated in vacuo. Purification by flash chromatography (24 g SiO₂, 0 to 100% EtOAc in hexanes) gave 6-((tert-butyldimethylsilyl)oxy)spiro[3.3]heptan-2-ol (180 mg, 32%) as a colourless oil. ¹H NMR (500 MHZ, Chloroform-d) δ 4.17-4.13 (m, 2H), 2.38-2.35 (m, 1H), 2.31-2.18 (m, 3H), 1.96-1.84 (m, 4H), 1.26 (m, 1H), 0.86 (s, 9H), 0.00 (s, 6H) ppm.

### Intermediate W

### 4-amino-5-fluoropyridin-2-yl) (4-methylpiperazin-1-yl) methanone

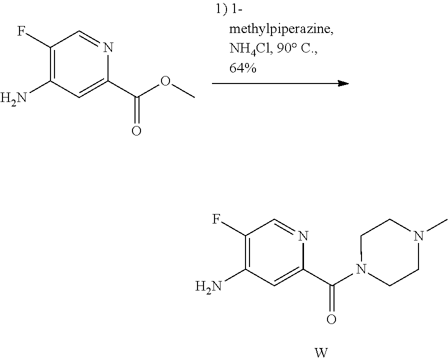

### Step 1:

A mixture of ethyl 4-amino-5-fluoropyridine-2-carboxylate (200 mg, 1.086 mmol), 1-methylpiperazine (451.50 mg, 0.5 mL, 4.508 mmol) and ammonium chloride (7 mg, 0.131 mmol) was stirred at 90° C. for 18 h and then, at 110° C. for 4 h. The reaction mixture was concentrated in vacuo. Purification by reversed phase HPLC-MS using a X-bridge C18 column (150×19 mm, 5 μm particle size) from Waters gave (4-amino-5-fluoropyridin-2-yl) (4-methylpiperazin-1-yl) methanone (165 mg, 64%) as a yellow oil. ¹H NMR (400 MHZ, Chloroform-d) δ 8.11 (dd, J=2.9, 1.5 Hz, 1H), 7.04 (d, J=7.3 Hz, 1H), 4.47-4.44 (m, 2H), 3.83-3.77 (m, 2H), 3.69-3.62 (m, 2H), 2.55-2.49 (m, 2H), 2.47-2.40 (m, 2H), 2.35-2.32 (m, 3H) ppm. ESI-MS m/z calc. 238.123, found 239.18 (M+1)⁺; Retention time: 0.34 minutes.

The following intermediates were made using a method similar to that described in Intermediate W except that different starting materials were used in place of ethyl 4-amino-5-fluoropyridine-2-carboxylate. In the case of W-2 and W-3, tert-butyl piperazine-1-carboxylate was used as the amine in place of 1-methylpiperazine. In the case of W-3 and W-4, the reaction was carried out at reflux in the presence of methanol as the solvent:

| Cmpd No. | Compound Name | LC/MS | NMR (shifts in ppm) |
|---|---|---|---|
| W-1 | (2-aminopyridin-4-yl)(4- | ESI-MS m/z calc. | ¹H NMR (400 MHz, |
| | methylpiperazin-1- | 220.1324, found 221.18 | Chloroform-d) δ 8.09 (d, |
| | yl)methanone | (M + 1)⁺; Retention time: | J = 5.0 Hz, 1H), 6.57 (dd, |
| | | 0.32 minutes | J = 5.0, 0.9 Hz, 1H), 6.45 |
| | | | (s, 1H), 4.59 (s, 2H), |
| | | | 3.80-3.74 (m, 2H), 3.42- |
| | | | 3.36 (m, 2H), 2.50-2.44 |
| | | | (m, 2H), 2.37-2.32 (m, |
| | | | 2H), 2.31 (s, 3H) ppm. |
| W-2 | tert-butyl 4-(2- | ESI-MS m/z calc. | ¹H NMR (400 MHz, |
| | aminoisonicotinoyl)piperazine- | 306.1692, found 307.33 | Chloroform-d) δ 8.10 (d, |
| | 1-carboxylate | (M + 1)⁺; Retention time: | J = 5.0 Hz, 1H), 6.58 (dd, |
| | | 0.59 minutes | J = 5.5, 1.4 Hz, 1H), 6.49 |
| | | | (s, 1H), 4.80 (br s, 2H), |
| | | | 3.75-3.68 (m, 2H), 3.54- |
| | | | 3.48 (m, 2H), 3.41-3.32 |
| | | | (m, 4H), 1.47 (s, 9H) |
| | | | ppm. |
| W-3 | tert-butyl 4-(6- | ESI-MS m/z calc. | ¹H NMR (400 MHz, |
| | aminopicolinoyl)piperazine-1- | 306.1692, found 251.16 | Chloroform-d) δ 7.50 (dd, |
| | carboxylate | (M − 55)+; Retention time: | J = 8.2, 7.3 Hz, 1H), 6.89 |
| | | 0.62 minutes | (d, J = 7.3 Hz, 1H), 6.52 |
| | | | (dd, J = 8.2, 0.9 Hz, 1H), |
| | | | 4.54 (br s, 2H), 3.74-3.70 |
| | | | (m, 2H), 3.53-3.48 (m, |
| | | | 4H), 3.45-3.42 (m, 2H), |
| | | | 1.46 (s, 9H) ppm. |
| W-4 | (6-aminopyridin-2-yl)(4- | ESI-MS m/z calc. | ¹H NMR (400 MHz, |
| | methylpiperazin-1- | 220.1324, found 221.21 | Chloroform-d) δ 7.49 (dd, |
| | yl)methanone | (M + 1)⁺; Retention time: | J = 8.2, 7.3 Hz, 1H), 6.86 |
| | | 0.35 minutes | (d, J = 7.3 Hz, 1H), 6.51 |
| | | | (d, J = 8.2 Hz, 1H), 4.56 |
| | | | (br s, 2H), 3.84-3.76 (m, |
| | | | 2H), 3.65-3.51 (m, 2H), |
| | | | 2.55-2.50 (m, 2H), 2.44- |
| | | | 2.39 (m, 2H), 2.32 (s, 3H) |
| | | | ppm. |

### Intermediate X

### tert-butyl 4-(6-aminopyrimidine-4-carbonyl) piperazine-1-carboxylate

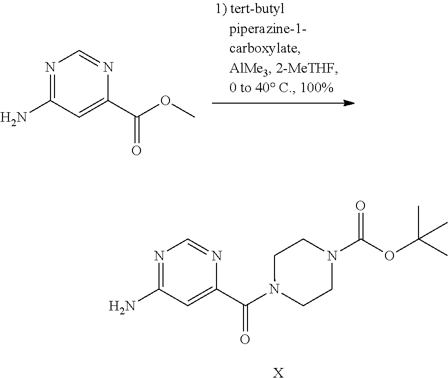

### Step 1:

Trimethylaluminum (0.6 mL, 2 M in heptanes, 1.2 mmol) was added to a stirred mixture of tert-butyl piperazine-1-carboxylate (223 mg, 1.197 mmol) in 2-MeTHF (2 mL) at 0° C. The reaction mixture was stirred for 15 min at ambient temperature and added to a mixture of methyl 6-aminopyrimidine-4-carboxylate (166 mg, 1.084 mmol) in 2-MeTHF (1 mL) at ambient temperature. The reaction mixture was stirred and heated at 40° C. for 1 h, cooled to ambient temperature and quenched by addition of methanol (0.1 mL). The mixture was partitioned between dichloromethane (5 mL) and a saturated aqueous ammonium chloride solution (5 mL). The organic extracts were separated, dried (phase separator cartridge) and concentrated in vacuo to give tert-butyl 4-(6-aminopyrimidine-4-carbonyl) piperazine-1-carboxylate (416 mg, 100%) as a beige solid. ¹H NMR (400 MHZ, Chloroform-d) δ 8.56 (s, 1H), 6.72 (s, 1H), 5.06 (br s, 2H), 3.73-3.70 (m, 2H), 3.58-3.55 (m, 2H), 3.53-3.51 (m, 2H), 3.47-3.45 (m, 2H), 1.46-1.48 (9H) ppm. ESI-MS m/z calc. 307.1644, found 306.04 (M−1); Retention time: 0.54 minutes.

The following intermediates were made using a method similar to that described in Intermediate X except that different starting materials were used in place of methyl 6-aminopyrimidine-4-carboxylate. For some of the intermediates, 1-methylpiperazine was used as the amine in place of tert-butyl piperazine-1-carboxylate. In the case of X-1 and X-2, the reaction was carried out in the presence of DCM as the solvent:

| Cmpd No. | Compound Name | LC/MS | NMR (shifts in ppm) |
|---|---|---|---|
| X-1 | (4-aminopyrimidin-2-yl)(4- | ESI-MS m/z calc. | ¹H NMR (400 MHz, |
| | methylpiperazin-1- | 221.1277, found 222.17 | Chloroform-d) δ 8.20 (d, |
| | yl)methanone | (M + 1)⁺; Retention time: | J = 6.0 Hz, 1H), 6.42 (d, |
| | | 0.27 minutes | J = 6.0 Hz, 1H), 5.52 (br s, |
| | | | 2H), 3.78 (t, J = 5.0 Hz, |
| | | | 2H), 3.47 (t, J = 5.0 Hz, |
| | | | 2H), 2.49 (t, J = 5.0 Hz, |
| | | | 2H), 2.41 (t, J = 5.0 Hz, |
| | | | 2H), 2.31 (s, 3H) ppm. |
| X-2 | (2-aminopyrimidin-4-yl)(4- | ESI-MS m/z calc. | ¹H NMR (400 MHz, |
| | methylpiperazin-1- | 221.1277, found 222.14 | Chloroform-d) δ 8.39 (d, |
| | yl)methanone | (M + 1)⁺; Retention time: | J = 5.0 Hz, 1H), 6.77 (d, |
| | | 0.31 minutes | J = 4.6 Hz, 1H), 5.12 (br s, |
| | | | 2H), 3.79-3.77 (m, 2H), |
| | | | 3.50 (t, J = 5.0 Hz, 2H), |
| | | | 2.49 (t, J = 5.0 Hz, 2H), |
| | | | 2.39 (t, J = 5.0 Hz, 2H), |
| | | | 2.32 (s, 3H) ppm. |
| X-3 | (4-amino-5-methylpyridin-2- | ESI-MS m/z calc. | ¹H NMR (400 MHz, |
| | yl)(4-methylpiperazin-1- | 234.1481, found 235.17 | Chloroform-d) δ 8.05 (s, |
| | yl)methanone | (M + 1)⁺; Retention time: | 1H), 6.86 (s, 1H), 4.19 |
| | | 0.36 minutes | (br s, 2H), 3.91-3.82 (m, |
| | | | 2H), 3.73-3.68 (m, 2H), |
| | | | 2.61-2.56 (m, 2H), 2.53- |
| | | | 2.47 (m, 2H), 2.38 (br s, |
| | | | 3H), 2.13 (s, 3H) ppm. |
| X-4 | tert-butyl 4-(4-amino-5- | ESI-MS m/z calc. | ¹H NMR (400 MHz, |
| | fluoropicolinoyl)piperazine-1- | 324.1598, found 323.13 | Chloroform-d) δ 8.13 (s, |
| | carboxylate | (M − 1)⁻; Retention time: | 1H), 7.15-7.09 (m, 1H), |
| | | 0.63 minutes | 4.75 (br s, 2H), 3.74-3.68 |
| | | | (m, 2H), 3.62-3.56 (m, |
| | | | 2H), 3.54-3.50 (m, 2H), |
| | | | 3.48-3.43 (m, 2H), 1.46 |
| | | | (s, 9H) ppm. |
| X-5 | tert-butyl 4-(4- | ESI-MS m/z calc. | ¹H NMR (400 MHz, |
| | aminopyrimidine-2- | 307.1644, found 306.04 | Chloroform-d) δ 8.24 (d, |
| | carbonyl)piperazine-1- | (M − 1)⁻; Retention time: | J = 6.0 Hz, 1H), 6.44 (d, |
| | carboxylate | 0.53 minutes | J = 6.0 Hz, 1H), 5.22 (br s, |
| | | | 2H), 3.75-3.72 (m, 2H), |
| | | | 3.53-3.50 (m, 2H), 3.47- |
| | | | 3.44 (m, 2H), 3.40-3.37 |
| | | | (m, 2H), 1.46 (s, 9H) |
| | | | ppm. |
| X-6 | tert-butyl 4-(2- | ESI-MS m/z calc. | ¹H NMR (400 MHz, |
| | aminopyrimidine-4- | 307.1644, found 308.13 | Chloroform-d) δ 8.40 (d, |
| | carbonyl)piperazine-1- | (M + 1)⁺; Retention time: | J = 5.0 Hz, 1H), 6.82 (d, |
| | carboxylate | 0.58 minutes | J = 5.0 Hz, 1H), 5.32 (br s, |
| | | | 2H), 3.73-3.70 (m, 2H), |
| | | | 3.53-3.51 (m, 2H), 3.49- |
| | | | 3.44 (m, 4H), 1.47 (s, 9H) |
| | | | ppm. |
| X-7 | tert-butyl 4-(4-amino-5- | ESI-MS m/z calc. | ¹H NMR (400 MHz, |
| | methylpicolinoyl)piperazine-1- | 320.1848, found 321.16 | Chloroform-d) δ 8.03 (s, |
| | carboxylate | (M + 1)⁺; Retention time: | 1H), 6.98 (s, 1H), 4.97 |
| | | 0.63 minutes | (br s, 2H), 3.73-3.69 (m, |
| | | | 2H), 3.57-3.45 (m, 6H), |
| | | | 2.14 (s, 3H), 1.46 (s, 9H) |
| | | | ppm |

### Example 30

### E-VIPR Assay Detecting and Measuring Naᵥ Inhibition Properties

Sodium ion channels are voltage-dependent proteins that can be activated by inducing membrane voltage changes by applying electric fields. The electrical stimulation instrument and methods of use, referred to as E-VIPR, are described in International Publication No. WO 2002/008748 A3 and C.-J. Huang et al. Characterization of voltage-gated sodium channel blockers by electrical stimulation and fluorescence detection of membrane potential, 24 Nature Biotech. 439-46 (2006), both of which are incorporated by reference in their entirety. The instrument comprises a microtiter plate handler, an optical system for exciting the coumarin dye while simultaneously recording the coumarin and oxonol emissions, a waveform generator, a current- or voltage-controlled amplifier, and parallel electrode pairs that are inserted into assay plate wells. Under integrated computer control, this instrument passes user-programmed electrical stimulus protocols to cells within the wells of the microtiter plate.

16-20 hours prior to running the assay on E-VIPR, HEK cells expressing a truncated form of human Naᵥ 1.8 with full channel activity were seeded into microtiter 384-well plates, pre-coated with matrigel, at a density of 25,000 cells per well. 2.5-5% KIR2.1 Bacmam virus was added to the final cell suspension before seeding into cell plates. HEK cells were grown in Dulbecco's Modified Eagle's Medium (DMEM) supplemented with 10% FBS (Fetal Bovine Serum, qualified; Sigma #F4135), 1% NEAA (Non-Essential Amino Acids, Gibco #11140), 1% HEPES (Gibco #15630), 1% Pen-Strep (Penicillin-Streptomycin; Gibco #15140) and 5 μg/ml Blasticidin (Gibco #R²¹⁰-01). Cells were expanded in vented cap cell culture flasks, with 90-95% humidity and 5% CO₂.

### Reagents and Stock Solutions:

100 mg/mL Pluronic F-127 (Sigma #P2443), in dry DMSO

Compound Plates: Corning 384-well Polypropylene Round Bottom #3656

Cell Plates: 384-well tissue culture treated plates (Greiner #781091-2B)

2.5-5% KIR 2.1 Bacmam virus (produced in-house), prepared as described in Section 3.3 of J. A. Fornwald et al., Gene Expression in Mammalian Cells Using BacMam, a Modified Baculovirus System, 1350 Methods in Molecular Biology 95-116 (2016), the entire contents of which are incorporated by reference. The concentration used can be dependent on viral titer of each batch.

5 mM DiSBAC₆(3), a voltage sensitive oxonol acceptor (CAS number 169211-44-3; 5-[3-(1,3-dihexylhexahydro-4,6-dioxo-2-thioxo-5-pyrimidinyl)-2-propen-1-ylidene]-1,3-dihexyldihydro-2-thioxo-4,6 (1H,5H)-pyrimidinedione), in dry DMSO. The preparation of DiSBAC₆(3) is analogous to that of DiSBAC₄(3) as described in Voltage Sensing by Fluorescence Resonance Energy Transfer in Single Cells, Gonzalez, J. E. and Tsien, R. Y. (1995) Biophys. J. 69, 1272-1280.

5 mM CC2-DMPE, a commercially available membrane-bound coumarin phospholipid FRET donor (ThermoFisher Scientific catalog number K₁₀₁₇, CAS number 393782-57-5; tetradecanoic acid, 1,1′-[(1R)-1-[8-(6-chloro-7-hydroxy-2-oxo-2H-1-benzopyran-3-yl)-3-hydroxy-3-oxido-8-oxo-2,4-dioxa-7-aza-3-phosphaoct-1-yl]-1,2-ethanediyl] ester) was prepared in dry DMSO. See also, Improved indicators of cell membrane potential that use fluorescence resonance energy transfer, Gonzalez, J. E. and Tsien, R. Y. (1997) Chem. Biol. 4, 269-277.

Voltage Assay Background Suppression Compound (VABSC-1) is prepared in H₂O (89-363 mM, range used to maintain solubility)

Human Serum (HS, Millipore #S1P1-01KL, or Sigma SLBR5469V and SLBR5470V as a 50%/50% mixture, for 25% assay final concentration)

### Bath 1 Buffer:

Sodium Chloride 160 mM (9.35 g/L), Potassium Chloride, 4.5 mM (0.335 g/L), Glucose 10 mM (1.8 g/L), Magnesium Chloride (Anhydrous) 1 mM (0.095 g/L), Calcium Chloride 2 mM (0.222 g/L), HEPES 10 mM (2.38 g/L) in water.

### Na/TMA Cl Bath 1 Buffer:

Sodium Chloride 96 mM (5.61 g/L), Potassium Chloride 4.5 mM (0.335 g/L), Tetramethylammonium (TMA)-Cl 64 mM (7.01 g/L), Glucose 10 mM (1.8 g/L), Magnesium Chloride (Anhydrous) 1 mM (0.095 g/L), Calcium Chloride 2 mM (0.222 g/L) HEPES 10 mM (2.38 g/L) in water.

### Hexyl Dye Solution (2× Concentration):

Bath 1 Buffer containing 0.5% β-cyclodextrin (made fresh prior to each use, Sigma #C4767), 8 μM CC2-DMPE and 2 μM DiSBAC₆(3). The solution was made by adding 10% Pluronic F127 stock equal to combined volumes of CC2-DMPE and DiSBAC₆(3). The order of preparation was first mix Pluronic and CC2-DMPE, then add DiSBAC₆(3), then while vortexing add Bath 1/B-Cyclodextrin.

Compound Loading Buffer (2× concentration): Na/TMA Cl Bath1 Buffer containing HS (omitted in experiments run in the absence of human serum (HS)) 50%, VABSC-1 1 mM, BSA 0.2 mg/ml (in Bath-1), KCl 9 mM, DMSO 0.625%.

### Assay Protocol (7 key Steps):

1) To reach the final concentration in each well, 375 nL of each compound was pre-spotted (in neat DMSO) into polypropylene compound plates at 240× desired final concentration from an intermediate stock concentration of 0.075 mM, in an 11 point dose response, 3-fold dilution, resulting in a top dose of 300 nM final concentration in the cell plate. Vehicle control (neat DMSO), and positive control (an established Naᵥ1.8 inhibitor, 25 μM final in assay in DMSO) were added manually to the outermost columns of each plate respectively. The compound plate was backfilled with 45 μL per well of Compound Loading Buffer resulting in a 240 fold dilution of compound following a 1:1 transfer of compound into the cell plate (see Step 6). Final DMSO concentration for all wells in the assay was 0.625% (0.75% DMSO was supplemented to the Compound Loading Buffer for a final DMSO concentration of 0.625%). This assay dilution protocol was adjusted to enable a higher dose range to be tested in the presence of HS or if the final assay volume was altered.
2) Hexyl Dye Solution was prepared.
3) Cell plates were prepared. On the day of the assay, the media was aspirated, and the cells were washed three times with 80 μL of Bath-1 buffer, maintaining 25 μL residual volume in each well.
4) 25 μL per well of Hexyl Dye Solution was dispensed into the cell plates. The cells were incubated for 20 minutes at room temperature or ambient conditions in darkness.
5) 45 μL per well of Compound Loading Buffer was dispensed into compound plates.
6) The cell plates were washed three times with 80 μL per well of Bath-1 Buffer, leaving 25 μL of residual volume. Then 25 μL per well from compound plate was transferred to each cell plate. The mixture was incubated for 30 minutes at room temperature/ambient conditions.
7) The cell plate containing compound was read on E-VIPR using the current-controlled amplifier to deliver stimulation wave pulses using a symmetrical biphasic waveform. The user-programmed electrical stimulus protocols were 1.25-4 Amps and 4 millisecond pulse width (dependent on electrode composition) were delivered at 10 Hz for 10 seconds. A pre-stimulus recording was performed for each well for 0.5 seconds to obtain the un-stimulated intensities baseline. The stimulatory waveform was followed by 0.5 seconds of post-stimulation recording to examine the relaxation to the resting state. All E-VIPR responses were measured at 200 Hz acquisition rate.

### Data Analysis:

Data were analyzed and reported as normalized ratios of emission intensities measured in the 460 nm and 580 nm channels. The response as a function of time was reported as the ratios obtained using the following formula:

$$R ( t ) = \frac{( {intensity}_{460 nm} )}{( {intensity}_{580 nm} )}$$

The data were further reduced (i.e. normalized) by calculating the initial (Ri) and final (Rf) ratios. These were the average ratio values during part or all of the pre-stimulation period and during sample points during the stimulation period. The fluorescence ratio (Rf/Ri) was then calculated and reported as a function of time.

Control responses were obtained by performing assays in the presence of the positive control, and in the absence of pharmacological agents (DMSO vehicle negative control). Responses to the negative (N) and positive (P) controls were calculated as above. The compound antagonist % activity A was then defined as:

$$A = \frac{X - N}{P - N} \times 100$$

where X is the ratio response of the test compound (i.e. the maximum amplitude of the ratio response or number of action potential peaks, at the beginning of the pulse train in the presence of the test compound). Using this analysis protocol, dose response curves were plotted and IC₅₀ values were generated for various compounds of the present invention as reported below.

Compounds having a measured IC₅₀ value less than 0.01 μM in the E-VIPR Assay described above include: 2, 3, 5, 6, 8, 9, 15, 34, 38, 39, 40, 43, 45, 46, 50, 51, 52, 53, 55, 56, 57, 58, 59, 60, 61, 62, 64, 65, 66, 67, 68, 69, 70, 72, 73, 75, 76, 77, 78, 80, 81, 82, 83, 85, 86, 88, 89, 90, 93, 95, 107, 109, 110, 111, 112, 119, 120, 121, 124, 125, 127, 130, 132, 133, 140, 144, 145, 147, 149, 153, 154, 156, 157, 158, 159, 160, 162, 163, 164, 165, 166, 167, 168, 169, 170, 173, 174, 175, 177, 178, 180, 182, 183, 184, 185, 186, 187, 188, 189, 190, 191, 192, 193, 194, 195, 196, 197, 202, 207, 211, 212, 213, 215, 216, 217, 218, 219, 220, 221, 222, 223, 224, 227, 228, 229, 230, 231, 232, 234, 235, 238, 239, 240, 241, 243, 244, 245, 247, 248, 249, 250, 254, 255, 256, 257, 258, 259, 260, 261, 262, 263, 264, 265, 268, 269, 271, 273, 274, 278, 280, 282, 283, 284, 286, 287, 288, 292, 293, 294, 295, 296, 297, 298, and 302.

Compounds having a measured IC₅₀ value less than 0.1 μM and greater than or equal to 0.01 μM in the E-VIPR Assay described above include: 7, 11, 12, 16, 17, 18, 19, 20, 21, 33, 42, 44, 47, 48, 49, 54, 63, 74, 79, 84, 87, 92, 96, 97, 98, 99, 100, 101, 102, 103, 104, 105, 108, 113, 114, 116, 117, 118, 122, 126, 128, 129, 131, 134, 135, 136, 138, 151, 152, 155, 161, 172, 181, 199, 200, 201, 203, 205, 206, 214, 225, 233, 236, 237, 242, 251, 252, 253, 266, 270, 272, 275, 276, 277, 279, 281, 289, 299, 300, and 301.

Compounds having a measured IC₅₀ value less than 1 μM and greater than or equal to 0.1 μM in the E-VIPR Assay described above include: 1, 10, 13, 14, 22, 23, 26, 28, 31, 32, 35*, 37*, 41, 71, 91, 94, 106, 115, 141, 143, 171, 179, 198, 204, 208, 226, 246, 267, 285, 290, and 291.

Compounds having a measured IC₅₀ value greater than or equal to 1 μM in the E-VIPR Assay described above include: 4, 24, 25, 27, 29, 30*, 36*, 123, 137, 139, 142, 146, 148, 150, 176, and 209.

Compound numbers followed by “*” indicate that the assay was performed in the presence of human serum, as described above.

Many modifications and variations of the embodiments described herein may be made without departing from the scope, as is apparent to those skilled in the art. The specific embodiments described herein are offered by way of example only.

1. A compound of formula (I) 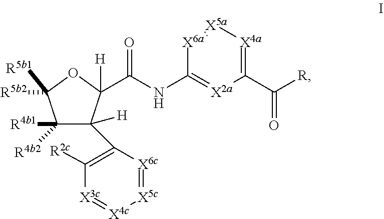
or a pharmaceutically acceptable salt thereof, wherein:
X^{2a} is N, N⁺—O⁻, or C—R^{2a};
X^{4a} is N, N⁺—O⁻, or C—R^{4a};
X^{5a} is N, N⁺—O⁻, or C—R^{5a};
X^{6a} is N, N⁺—O⁻, or C—R^{6a};
R is OR^{a} or NR^{Xa}R^{Ya};
R^{2a}, R^{4a}, R^{5a}, and R^{6a} are each independently H, halo, C₁-C₆ alkyl, C₁-C₆ haloalkyl, or —Si(C₁-C₆ alkyl)₃;
R^{a} is H or C₁-C₆ alkyl;
R^{Xa} is H or C₁-C₆ alkyl;
R^{Ya} is H, OH, C₁-C₆ alkyl, —(C₁-C₆ alkylene)-R^{Za1}, or 4-6 membered heterocyclyl optionally substituted with one or more groups independently selected from C₁-C₆ alkyl and C₁-C₆ alkoxy;
or R^{Xa} and R^{Ya}, together with the nitrogen atom to which they are attached, form a 5-9 membered heterocyclyl, wherein said heterocyclyl is optionally substituted with one or more R^{Za2};
R^{Za1} is OH, NH₂, —NH(C₁-C₆ alkyl), —N(C₁-C₆ alkyl)₂, and 5-6 membered heterocyclyl optionally substituted with one or more groups independently selected from halo and C₁-C₆ alkyl;
each R^{Za2} is independently selected from halo, OH, C₁-C₆ alkyl, C₁-C₆ alkoxy, NH₂, —NH(C₁-C₆ alkyl), —N(C₁-C₆ alkyl)₂, and —(C₁-C₆ alkylene)-(C₁-C₆ alkoxy);
R^{4b1} and R^{4b2} are each independently H, C₁-C₆ alkyl, C₃-C₆ cycloalkyl, or C₁-C₆ haloalkyl;
R^{5b1} and R^{5b2} are each independently H, C₁-C₆ alkyl, C₃-C₆ cycloalkyl, C₁-C₆ haloalkyl, or —(C₁-C₆ alkylene)-(C₁-C₆ alkoxy);
or R^{5b1} and R⁵⁶², together with the carbon atom to which they are attached, form a 4-6 membered heterocyclyl;
X^{3c} is N or C—R^{3c};
X^{4c} is N or C—R⁴;
X^{5c} is N or C—R^{5c};
X^{6c} is N or C—R⁶⁰;
R^{2c} is H, OH, halo, C₁-C₆ alkyl, C₂-C₆ alkenyl, C₁-C₆ haloalkyl, C₁-C₆ alkoxy, C₁-C₆ haloalkoxy, —(C₁-C₆ alkylene)-(C₁-C₆ alkoxy), —(C₁-C₆ alkylene)-O-(4-6 membered heterocyclyl), —O—(C₂-C₆ alkenylene)-(C₁-C₆ haloalkyl), -L¹-L²-(C₃-C₇ cycloalkyl), or —O-L³-R^{Xc}, wherein said cycloalkyl is optionally substituted with one or more groups independently selected from halo, OH, CN, C₁-C₆ alkyl, C₁-C₆ alkoxy, ═NOH, —C(O) (C₁-C₆ alkyl), and —(C₁-C₆ alkylene)-OH;
L¹ is a bond or O;
L² is a bond or C₁-C₆ alkylene;
L³ is a bond, C₁-C₆ alkylene, or C₂-C₆ alkenylene;
R^{Xc} is selected from OH, CN, C₁-C₆ alkoxy, NH₂, —NH(C₁-C₆ alkyl), —N(C₁-C₆ alkyl)₂, —NH(C₁-C₆ haloalkyl), —NH(C₁-C₆ haloalkyl)₂, —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O) (C₁-C₆ alkyl), —C(O) NH₂, —C(O) NH(C₁-C₆ alkyl), —C(O) N(C₁-C₆ alkyl)₂, —NH (4-6 membered heterocyclyl), ═NOH, ═NO(C₁-C₆ alkyl), —N═S(O)(C₁-C₆ alkyl)₂, —C(═NOH)(C₃-C₆ cycloalkyl), 4-8 membered heterocyclyl, and 5-6 membered heteroaryl, wherein said cycloalkyl is optionally substituted with one or more halo, and wherein said heterocyclyl and heteroaryl are optionally substituted with one or more groups independently selected from OH, halo, C₁-C₆ alkyl, C₁-C₆ alkoxy, C₁-C₆ haloalkyl, C₁-C₆ haloalkoxy, and —(C₁-C₆ alkylene)-OH;
R^{3c} is H, halo, C₁-C₆ alkyl, C₁-C₆ haloalkyl, —(C₁-C₆ alkylene)-OH, or —(C₁-C₆ alkylene)-(C₁-C₆ alkoxy);
or wherein X^{3c} is C—R^{3c}, and R^{2c} and R^{3c}, together with the carbon atoms to which they are attached, form a ring of formula: 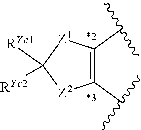
Z¹ and Z² are each, independently, O, CH₂, or CF₂;
R^{Yc1} and RYe2 are each, independently, H or halo;
R⁴ is H, halo, OH, —OBn, C₁-C₆ alkyl, C₁-C₆ alkoxy, C₁-C₆ haloalkyl, C₁-C₆ haloalkoxy, or -L¹-L²-(C₃-C₆ cycloalkyl), wherein said cycloalkyl is optionally substituted with 1-2 halo;
R^{5c} is H, halo, OH, —OBn, C₁-C₆ alkyl, C₁-C₆ haloalkyl, or -L¹-L²-(C₃-C₆ cycloalkyl), wherein said cycloalkyl is optionally substituted with 1-2 halo;
R^{6c} is H, halo, C₁-C₆ alkyl, or C₁-C₆ haloalkyl;
provided that no more than two of X^{2a}, X^{4a}, X^{5a}, and X^{6a} are N or N⁺—O⁻;
provided that no more than one of X^{3c}, X^{4c}, X^{5c}, and X^{6c} is N; and
provided that:
R is OR^{a}; or
R is NR^{Xa}R^{Ya}, wherein R^{Ya} is OH, —(C₁-C₆ alkylene)-R^{Za1}, or 4-6 membered heterocyclyl optionally substituted with one or more groups independently selected from C₁-C₆ alkyl and C₁-C₆ alkoxy; or
R is NR^{Xa}R^{Ya}, wherein R^{Xa} and R^{Ya}, together with the N atom to which they are attached, form a 5-9 membered heterocyclyl, and wherein said heterocyclyl is optionally substituted with one or more R^{Za2}; or
R^{2a}, R^{4a}, R^{5a}, or R^{6a} is —Si(C₁-C₆ alkyl); or
R^{5b1} or R^{5b2} is —(C₁-C₆ alkylene)-(C₁-C₆ alkoxy); or
R^{5b1} and R^{5b2}, together with the carbon atom to which they are attached, form a 4-6 membered heterocyclyl; or
R^{2c} is —(C₁-C₆ alkylene)-(C₁-C₆ alkoxy), —(C₁-C₆ alkylene)-O-(4-6 membered heterocyclyl), —O—(C₂-C₆ alkenylene)-(C₁-C₆ haloalkyl), or —O-L³-R^{Xc}; or
R^{2c} is -L¹-L²-(C₃-C₇ cycloalkyl), wherein said cycloalkyl is substituted with one or more groups independently selected from OH, CN, C₁-C₆ alkyl, C₁-C₆ alkoxy, ═NOH, —C(O) (C₁-C₆ alkyl), and —(C₁-C₆ alkylene)-OH; or
R^{3c} is —(C₁-C₆ alkylene)-OH or —(C₁-C₆ alkylene)-(C₁-C₆ alkoxy); or
R^{2c} and R^{3c}, together with the carbon atoms to which they are attached, form a ring of formula: 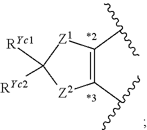
or
R^{4c} is OH, —OBn, C₁-C₆ alkoxy, C₁-C₆ haloalkoxy, or -L¹-L²-(C₃-C₆ cycloalkyl), wherein said cycloalkyl is optionally substituted with 1-2 halo; or
R^{5c} is OH, —OBn, or -L¹-L²-(C₃-C₆ cycloalkyl), wherein said cycloalkyl is optionally substituted with 1-2 halo.

2. (canceled)

3. (canceled)

4. The compound of claim 1, wherein the compound has formula (I-B) or (I-C) 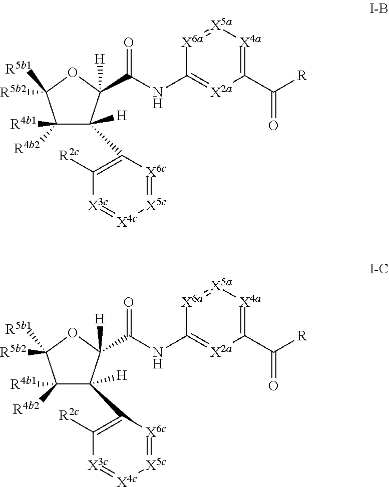
or a pharmaceutically acceptable salt thereof.

5. (canceled)

6. The compound of claim 1, or the pharmaceutically acceptable salt thereof, wherein X^{2a} is N or C—R^{2a}, and R^{2a} is H.

7. The compound of claim 1, or the pharmaceutically acceptable salt thereof, wherein X^{4a} is N, N⁺—O⁻, or C—R^{4a}; and R^{4a} is H or halo, optionally F.

8. The compound of claim 1, or the pharmaceutically acceptable salt thereof, wherein X^{5a} is C—R^{5a}, and R^{5a} is H.

9. The compound of claim 1, or the pharmaceutically acceptable salt thereof, wherein X^{6a} is N or C—R^{6a}; and R^{6a} is H, halo, optionally F, C₁-C₆ alkyl, optionally CH₃, or —Si(C₁-C₆ alkyl)₃, optionally Si(CH₃)₃.

10. The compound of claim 1, or the pharmaceutically acceptable salt thereof, wherein:
R is OR^{a} or NR^{Xa}R^{Ya};
R^{a} is H or C₁-C₆ alkyl;
R^{Xa} is H or C₁-C₆ alkyl;
R^{Ya} is H, OH, C₁-C₆ alkyl, —(C₁-C₆ alkylene)-R^{Za1}, or 4-6 membered heterocyclyl optionally substituted with one or more groups independently selected from C₁-C₆ alkyl and C₁-C₆ alkoxy, optionally CH₃, —OCH₃, or —OCH₂CH₃; and
R^{Za1} is OH, —NH(C₁-C₆ alkyl), —N(C₁-C₆ alkyl)₂, or 5-6 membered heterocyclyl optionally substituted with one or more groups independently selected from halo and C₁-C₆ alkyl; or
R is NR^{Xa}R^{Ya},
R^{Xa} and R^{Ya}, together with the nitrogen atom to which they are attached, form a 5-9 membered heterocyclyl optionally substituted with one or more R^{Za2}; and
each R^{Za2} is independently selected from halo, OH, C₁-C₆ alkyl,
C₁-C₆ alkoxy, NH₂, —NH(C₁-C₆ alkyl), —N(C₁-C₆ alkyl)₂, and —(C₁-C₆ alkylene)-(C₁-C₆ alkoxy).

11. (canceled)

12. The compound of claim 1, or the pharmaceutically acceptable salt thereof, wherein R^{4b1} is H or C₁-C₆ alkyl, optionally CH₃ and/or R^{4b2} is H or C₁-C₆ alkyl.

13. (canceled)

14. The compound of claim 1, or the pharmaceutically acceptable salt thereof, wherein R^{5b1} is C₁-C₆ alkyl, C₁-C₆ haloalkyl, or —(C₁-C₆ alkylene)-(C₁-C₆ alkoxy), and/or R^{5b2} is C₁-C₆ alkyl, C₁-C₆ haloalkyl, or —(C₁-C₆ alkylene)-(C₁-C₆ alkoxy); or R^{5b1} and R⁵⁶², together with the carbon atom to which they are attached, form a 4-membered heterocyclyl.

15. (canceled)

16. (canceled)

17. The compound of claim 1, or the pharmaceutically acceptable salt thereof, wherein X^{3c} is N, or X^{3c} is C—R^{3c}; and R^{3c} is H, halo, C₁-C₆ alkyl, C₁-C₆ haloalkyl, —(C₁-C₆ alkylene)-OH, or —(C₁-C₆ alkylene)-(C₁-C₆ alkoxy).

18. (canceled)

19. The compound of claim 1, or the pharmaceutically acceptable salt thereof, wherein X^{4c} is C—R^{4c}; R⁴ is H, halo, OH, —OBn, C₁-C₆ alkoxy, C₁-C₆ haloalkyl, C₁-C₆ haloalkoxy, or -L¹-L²-(C₃-C₆ cycloalkyl), wherein said cycloalkyl is optionally substituted with 1-2 halo; L¹ is O; and L² is a bond or C₁-C₆ alkylene.

20. (canceled)

21. (canceled)

22. The compound of claim 1, or the pharmaceutically acceptable salt thereof, wherein X^{5c} is C—R^{5c}; and R^{5c} is H, halo, OH, —OBn, or -L¹-L²-(C₃-C₆ cycloalkyl), wherein said cycloalkyl is optionally substituted with 1-2 halo; L¹ is O; and L² is a bond.

23. (canceled)

24. (canceled)

25. The compound of claim 1, or the pharmaceutically acceptable salt thereof, wherein X^{6c} is C—R⁶⁰; and R^{6c} is H.

26. The compound of claim 1, or the pharmaceutically acceptable salt thereof, wherein:
R^{2c} is OH, halo, optionally C₁, C₁-C₆ alkoxy, optionally-OCH₃, —(C₁-C₆ alkylene)-(C₁-C₆ alkoxy), optionally-CH₂OCH₃, —(C₁-C₆ alkylene)-O-(4-6 membered heterocyclyl), optionally —CH₂—O-(4-membered heterocyclyl), —O—(C₂-C₆ alkenylene)-(C₁-C₆ haloalkyl), optionally —O—(C₃-C₄ alkenylene)-CF₃, -L¹-L²-(C₃-C₇ cycloalkyl), or —O-L³-R^{Xc}, wherein said cycloalkyl is optionally substituted with one or more groups independently selected from OH, CN, C₁-C₆ alkyl, C₁-C₆ alkoxy, ═NOH, —C(O) (C₁-C₆ alkyl), and —(C₁-C₆ alkylene)-OH;
L¹ is O;
L² is a bond or C₁-C₆ alkylene;
L³ is a bond, C₁-C₆ alkylene, or C₂-C₆ alkenylene; and
R^{Xc} is selected from OH, CN, C₁-C₆ alkoxy, NH₂, —NH(C₁-C₆ alkyl), —N(C₁-C₆ alkyl)₂, —NH(C₁-C₆ haloalkyl), —NH(C₁-C₆ haloalkyl)₂, —CH(CH₂OH)₂, —CH(CH₂OH) (CH₂OCH₃), —CH(CH₂OH) (OCH₃), —CH(CH₂OCH₃) (OCH₃), —CH(CH₂OH)(CF₃), —C(O) (C₁-C₆ alkyl), —C(O) NH₂, —C(O) NH(C₁-C₆ alkyl), —C(O) N(C₁-C₆ alkyl)₂, —NH (4-6 membered heterocyclyl), ═NOH, ═NO(C₁-C₆ alkyl), —N═S(O)(C₁-C₆ alkyl)₂, —C(═NOH)(C₃-C₆ cycloalkyl), 4-8 membered heterocyclyl, and 5-6 membered heteroaryl, wherein said cycloalkyl is optionally substituted with one or more halo, and wherein said heterocyclyl and heteroaryl are optionally substituted with one or more groups independently selected from OH, halo, C₁-C₆ alkyl, C₁-C₆ alkoxy, C₁-C₆ haloalkyl, C₁-C₆ haloalkoxy, and —(C₁-C₆ alkylene)-OH.

27. (canceled)

28. (canceled)

29. (canceled)

30. The compound of claim 1, or the pharmaceutically acceptable salt thereof, wherein X^{3c} is C—R^{3c}, and R^{2c} and R^{3c}, together with the carbon atoms to which they are attached, form a ring of formula: 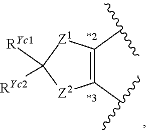
and wherein:
Z¹ is O or CH₂;
Z² is O or CF₂; and
R^{Yc1} and R^{Yc2} are each, independently, H or F.

31. (canceled)

32. A compound selected from Table A or Table B, or a pharmaceutically acceptable salt thereof.

33. (canceled)

34. A pharmaceutical composition comprising a therapeutically effective amount of the compound of claim 1, or a pharmaceutically acceptable salt thereof, or the compound of claim 33, and one or more pharmaceutically acceptable carriers or vehicles.

35. (canceled)

36. A method of inhibiting a voltage-gated sodium channel, optionally wherein the voltage-gated sodium channel is Naᵥ1.8, in a subject comprising administering to the subject the compound of claim 1, or a pharmaceutically acceptable salt thereof.

37. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of claim 1, or a pharmaceutically acceptable salt thereof.

38. The method of claim 37, where the method comprises treating or lessening the severity in one or more of the subject of: neuropathic pain; musculoskeletal pain, optionally osteoarthritis pain; acute pain; postsurgical pain; or visceral pain.

39. (canceled)

40. (canceled)

41. (canceled)

42. (canceled)
